(19) United States
(12) Patent Application Publication
(10) Pub. No.: US 2026/0209386 A1
(43) Pub. Date: Jul. 23, 2026

(54) PTK7-BINDING PROTEINS WITH PH-DEPENDENT BINDING AND USES THEREOF

(71) Applicant: Mythic Therapeutics, Inc., Waltham, MA (US)

(72) Inventors: Nimish Gera, Waltham, MA (US); Brian P. Fiske, Waltham, MA (US); Alexander J. Nichols, Lincoln, MA (US); Lihui Xu, Chestnut Hill, MA (US)

(73) Assignee: Mythic Therapeutics, Inc., Waltham, MA (US)

(21) Appl. No.: 18/865,394

(22) PCT Filed: May 19, 2023
(86) PCT No.: PCT/US2023/067229

§ 371 Date: Nov. 13, 2024

Related U.S. Application Data
(60) Provisional application No. 63/365,005, filed on May 19, 2022.

(51) Int. Cl. C07K 16/40; A61K 39/00; A61K 47/68; A61P 35/00

(52) U.S. Cl. CPC C07K 16/40; A61K 47/6817; A61K 47/6871; A61P 35/00; A61K 2039/505; C07K 2317/56; C07K 2317/565; C07K 2317/77; C07K 2317/92; C07K 2317/94

(57) ABSTRACT

Provided herein are antigen-binding protein constructs (ABPCs) that bind PTK7 and uses of the same.

### CROSS-REFERENCE TO RELATED APPLICATIONS

This application claims priority to U.S. Provisional Patent Application Ser. No. 63/365,005, filed May 19, 2022; the entire contents of which are herein incorporated by reference.

### SEQUENCE LISTING

This application contains a Sequence Listing that has been submitted electronically as an XML file named “45395-0057WO1_SL_ST26.XML”. The XML file, created on May 15, 2023, is 1,242,742 bytes in size. The material in the XML file is hereby incorporated by reference in its entirety.

### REFERENCE TO RELATED APPLICATIONS

The present disclosure relates to the field of antigen-binding molecules.

### TECHNICAL FIELD

The present disclosure relates to the field of antigen-binding molecules.

### SUMMARY

The present invention is based on the concept that antigen-binding protein (ABP) constructs can be generated that display enhanced efficacy in toxin liberation in a target mammalian cell, an increase in cell killing, and an increase in endolysosomal delivery.

Tyrosine-protein kinase-like 7 (PTK7), also known as colon carcinoma kinase 4 (CCK4), is a receptor tyrosine kinase that in humans is encoded by the PTK7 gene. “PTK7” and “CCK4” are used herein interchangeably. PTK7 is overexpressed in a number of large cancers (e.g. ovarian, breast, and non-small cell lung, and colon cancer), and the anti-PTK7 ADC, Cofetuzumab pelidotin, is currently being evaluated in clinical trials. Reasoning that pH engineering of PTK7 antibodies could potentially reduce uptake of corresponding ADCs in normal tissues, applicant sought to develop the anti-PTK7 binding proteins as disclosed herein. Such anti-PTK7 pH-ADCs hold the potential to mitigate the “on target” toxicity and widen therapeutic window as compared to non-pH-dependent anti-PTK7 ADCs. The pH-ADCs of the present disclosure could also improve efficacy by increasing tumor uptake and improving the PK profile, which could be particularly beneficial for patients having tumors characterized by low PTK7 expression.

As disclosed herein, applicant conducted a de novo antibody screening campaign using the extracellular domain of PTK7 to immunize rabbits. From hundreds of initial clones, applicant selected a diverse set of PTK7-binding proteins for subsequent pH-engineering. As detailed herein, applicant has produced over 500 variants of these initial rabbit monoclonal antibody (RabmAb) clones, and tested same for their ability to exhibit greater PTK7-specific binding at pH 7.4 vs. pH 5.4 as well as increased internalization by cancer cells expressing PTK7 on their surfaces. Accordingly, provided herein are antigen-binding protein construct (ABPCs) and pharmaceutical compositions including an effective amount of such ABPCs including: a first antigen-binding domain (ABD) that is capable of specifically binding PTK7 or an epitope of PTK7 presented on the surface of a target mammalian cell, where: (a) the dissociation rate of the first ABD at a pH of about 4.0 to about 6.5 is faster than the dissociation rate at a pH of about 7.0 to about 8.0; and/or (b) the dissociation constant (K_{D}) of the first ABD at a pH of about 4.0 to about 6.5 is greater than the K_{D} at a pH of about 7.0 to about 8.0. In particular embodiments. In addition to producing the foregoing anti-PTK7 antibodies and variants thereof, each disclosed herein for the first time, applicant also produced hundreds of variants beginning with parent PTK7-binding proteins selected from cofetuzumab, 7C8, and 12C6.

In some embodiments, the first ABD includes a heavy chain variable domain (HCVD) of cofetuzumab, 7C8, 12C6, MYT9345, MYT9359, MYT9361, MYT9412, MYT9460, MYT9792, or MYT9797, each HCVD optionally with at least 1 histidine substitution.

In some embodiments, the first ABD includes a light chain variable domain (LCVD) of cofetuzumab, 7C8, 12C6, MYT9345, MYT9359, MYT9361, MYT9412, MYT9460, MYT9792, or MYT9797, each LCVD optionally with at least 1 histidine substitution.

In some embodiments, the first ABD includes one of: (a) an HCVD of MYT9345 and/or an LCVD of MYT9345; (b) an HCVD of MYT9359 and/or an LCVD of MYT9359; (c) an HCVD of MYT9361 and/or an LCVD of MYT9361; (d) an HCVD of MYT9412 and/or an LCVD of MYT9412; (e) an HCVD of MYT9460 and/or an LCVD of MYT9460; (f) an HCVD of MYT9792 and/or an LCVD of MYT9792; (g) an HCVD of MYT9797 and/or an LCVD of MYT9797, (h) an HCVD of cofetuzumab and/or an LCVD of cofetuzumab, (i) an HCVD of 7C8 and/or an LCVD of 7C8, and (j) an HCVD of 12C6 and/or an LCVD of 12C6; each HCVD or LCVD optionally with one or more amino acids substituted with a histidine.

In some embodiments, the HCVD includes one of: (a) an HCVD of MYT9345 including SEQ ID NO: 301; (b) an HCVD of MYT9359 including SEQ ID NO: 375; (c) an HCVD of MYT9361 including SEQ ID NO: 459; (d) an HCVD of MYT9412 including SEQ ID NO: 542; (e) an HCVD of MYT9460 including SEQ ID NO: 628; (f) an HCVD of MYT9792 including SEQ ID NO: 710; (g) an HCVD of MYT9797 including SEQ ID NO: 803; (h) an HCVD of cofetuzumab including SEQ ID NO: 1; (i) an HCVD of 7C8 including SEQ ID NO: 126; and (j) an HCVD of 12C6 including SEQ ID NO: 229.

In some embodiments, the LCVD includes one of: (a) an LCVD of MYT9345 including SEQ ID NO: 302; (b) an LCVD of MYT9359 including SEQ ID NO: 376; (c) an LCVD of MYT9361 including SEQ ID NO: 460; (d) an LCVD of MYT9412 including SEQ ID NO: 543; (e) an LCVD of MYT9460 including SEQ ID NO: 629; (f) an LCVD of MYT9792 including SEQ ID NO: 711; and (g) an LCVD of MYT9797 including SEQ ID NO: 804; (h) an LCVD of cofetuzumab including SEQ ID NO: 1; (i) an LCVD of 7C8 including SEQ ID NO: 127; and (j) an LCVD of 12C6 including SEQ ID NO: 229.

In some embodiments, the first ABD includes an HCVD of one of: (a) an HCVD including a CDR1, a CDR2, and a CDR3 of SEQ ID NOs: 303, 304, and 305, respectively, optionally with collectively a total of one or more amino acid positions in SEQ ID NOs: 303, 304, and 305 substituted with a histidine; (b) an HCVD including a CDR1, a CDR2, and a CDR3 of SEQ ID NOs: 377, 378, and 379, respectively, optionally with collectively a total of one or more amino acid positions in SEQ ID NOs: 377, 378, and 379 substituted with a histidine; (c) an HCVD including a CDR1, a CDR2, and a CDR3 of SEQ ID NOs: 461, 462, and 463, respectively, optionally with collectively a total of one or more amino acid positions in SEQ ID NOs: 461, 462, and 463 substituted with a histidine; (d) an HCVD including a CDR1, a CDR2, and a CDR3 of SEQ ID NOs: 544, 545, and 546, respectively, optionally with collectively a total of one or more amino acid positions in SEQ ID NOs: 544, 545, and 546 substituted with a histidine; (e) an HCVD including a CDR1, a CDR2, and a CDR3 of SEQ ID NOs: 630, 631, and 632, respectively, optionally with collectively a total of one or more amino acid positions in SEQ ID NOs: 630, 631, and 632 optionally substituted with a histidine; (f) an HCVD including a CDR1, a CDR2, and a CDR3 of SEQ ID NOs: 712, 713, and 714, respectively, optionally with collectively a total of one or more amino acid positions in SEQ ID NOs: 712, 713, and 714 substituted with a histidine; (g) an HCVD including a CDR1, a CDR2, and a CDR3 of SEQ ID NOs: 805, 806, and 807, respectively, optionally with collectively a total of one or more amino acid positions in SEQ ID NOs: 805, 806, and 807 optionally substituted with a histidine; (h) an HCVD including a CDR1, a CDR2, and a CDR3 of SEQ ID NOs: 3, 4, and 5, respectively, optionally with collectively a total of one or more amino acid positions in SEQ ID NOs: 3, 4, and 5 optionally substituted with a histidine; (i) an HCVD including a CDR1, a CDR2, and a CDR3 of SEQ ID NOs: 128, 129, and 130, respectively, optionally with collectively a total of one or more amino acid positions in SEQ ID NOs: 128, 129, and 130 optionally substituted with a histidine; and (j) an HCVD including a CDR1, a CDR2, and a CDR3 of SEQ ID NOs: 231, 232, and 233, respectively, optionally with collectively a total of one or more amino acid positions in SEQ ID NOs: 231, 232, and 233 substituted with a histidine.

In some embodiments, the first ABD includes an LCVD of one of: (a) an LCVD including a CDR1, a CDR2, and a CDR3 of SEQ ID NOs: 306, 307, and 308, respectively, optionally with collectively a total of one or more amino acid positions in SEQ ID NOs: 306, 307, and 308 optionally substituted with a histidine; (b) an LCVD including a CDR1, a CDR2, and a CDR3 of SEQ ID NOs: 380, 381, and 382, respectively, optionally with collectively a total of one or more amino acid positions in SEQ ID NOs: 380, 381, and 382 optionally substituted with a histidine; (c) an LCVD including a CDR1, a CDR2, and a CDR3 of SEQ ID NOs: 464, 465, and 466, respectively, optionally with collectively a total of one or more amino acid positions in SEQ ID NOs: 464, 465, and 466 substituted with a histidine; (d) an LCVD including a CDR1, a CDR2, and a CDR3 of SEQ ID NOs: 547, 548, and 549, respectively, optionally with collectively a total of one or more amino acid positions in SEQ ID NOs: 547, 548, and 549 optionally substituted with a histidine; (e) an LCVD including a CDR1, a CDR2, and a CDR3 of SEQ ID NOs: 633, 634, and 635, respectively, optionally with collectively a total of one or more amino acid positions in SEQ ID NOs: 633, 634, and 635 substituted with a histidine; (f) an LCVD including a CDR1, a CDR2, and a CDR3 of SEQ ID NOs: 715, 716, and 717, respectively, optionally with collectively a total of one or more amino acid positions in SEQ ID NOs: 715, 716, and 717 substituted with a histidine; (g) an LCVD including a CDR1, a CDR2, and a CDR3 of SEQ ID NOs: 808, 809, and 810, respectively, optionally with collectively a total of one or more amino acid positions in SEQ ID NOs: 808, 809, and 810 optionally substituted with a histidine; (h) an LCVD including a CDR1, a CDR2, and a CDR3 of SEQ ID NOs: 6, 7, and 8, respectively, optionally with collectively a total of one or more amino acid positions in SEQ ID NOs: 6, 7, and 8 optionally substituted with a histidine; (i) an LCVD including a CDR1, a CDR2, and a CDR3 of SEQ ID NOs: 131, 132, and 133, respectively, optionally with collectively a total of one or more amino acid positions in SEQ ID NOs: 131, 132, and 133 optionally substituted with a histidine; and (j) an LCVD including a CDR1, a CDR2, and a CDR3 of SEQ ID NOs: 234, 235, and 236, respectively, optionally with collectively a total of one or more amino acid positions in SEQ ID NOs: 234, 235, and 236 substituted with a histidine.

In some embodiments, the first ABD includes one of: (a) an HCVD including a CDR1, a CDR2, and a CDR3 of SEQ ID NOs: 303, 304, and 305, respectively, optionally with collectively a total of one or more amino acid positions in SEQ ID NOs: 303, 304, and 305 substituted with a histidine; and/or an LCVD including a CDR1, a CDR2, and a CDR3 of SEQ ID NOs: 306, 307, and 308, respectively, optionally with collectively a total of one or more amino acid positions in SEQ ID NOs: 306, 307, and 308 optionally substituted with a histidine; (b) an HCVD including a CDR1, a CDR2, and a CDR3 of SEQ ID NOs: 377, 378, and 379, respectively, optionally with collectively a total of one or more amino acid positions in SEQ ID NOs: 377, 378, and 379 optionally substituted with a histidine; and/or an LCVD including a CDR1, a CDR2, and a CDR3 of SEQ ID NOs: 380, 381, and 382, respectively, optionally with collectively a total of one or more amino acid positions in SEQ ID NOs: 380, 381, and 382 optionally substituted with a histidine; (c) an HCVD including a CDR1, a CDR2, and a CDR3 of SEQ ID NOs: 461, 462, and 463, respectively, optionally with collectively a total of one or more amino acid positions in SEQ ID NOs: 461, 462, and 463 substituted with a histidine; and/or an LCVD including a CDR1, a CDR2, and a CDR3 of SEQ ID NOs: 464, 465, and 466, respectively, optionally with collectively a total of one or more amino acid positions in SEQ ID NOs: 464, 465, and 466 substituted with a histidine; (d) an HCVD including a CDR1, a CDR2, and a CDR3 of SEQ ID NOs: 544, 545, and 546, respectively, optionally with collectively a total of one or more amino acid positions in SEQ ID NOs: 544, 545, and 546 optionally substituted with a histidine; and/or an LCVD including a CDR1, a CDR2, and a CDR3 of SEQ ID NOs: 547, 548, and 549, respectively, optionally with collectively a total of one or more amino acid positions in SEQ ID NOs: 547, 548, and 549 optionally substituted with a histidine; (e) an HCVD including a CDR1, a CDR2, and a CDR3 of SEQ ID NOs: 630, 631, and 632, respectively, optionally with collectively a total of one or more amino acid positions in SEQ ID NOs: 630, 631, and 632 optionally substituted with a histidine; and/or an LCVD including a CDR1, a CDR2, and a CDR3 of SEQ ID NOs: 633, 634, and 635, respectively, optionally with collectively a total of one or more amino acid positions in SEQ ID NOs: 633, 634, and 635 substituted with a histidine; (f) an HCVD including a CDR1, a CDR2, and a CDR3 of SEQ ID NOs: 712, 713, and 714, respectively, optionally with collectively a total of one or more amino acid positions in SEQ ID NOs: 712, 713, and 714 substituted with a histidine; and/or an LCVD including a CDR1, a CDR2, and a CDR3 of SEQ ID NOs: 715, 716, and 717, respectively, optionally with collectively a total of one or more amino acid positions in SEQ ID NOs: 715, 716, and 717 substituted with a histidine; (g) an HCVD including a CDR1, a CDR2, and a CDR3 of SEQ ID NOs: 805, 806, and 807, respectively, optionally with collectively a total of one or more amino acid positions in SEQ ID NOs: 805, 806, and 807 optionally substituted with a histidine; and/or an LCVD including a CDR1, a CDR2, and a CDR3 of SEQ ID NOs: 808, 809, and 810, respectively, optionally with collectively a total of one or more amino acid positions in SEQ ID NOs: 808, 809, and 810 optionally substituted with a histidine; (h) an HCVD including a CDR1, a CDR2, and a CDR3 of SEQ ID NOs: 3, 4, and 5, respectively, with collectively a total of one or more amino acid positions in SEQ ID NOs: 3, 4, and 5 substituted with a histidine; and/or an LCVD including a CDR1, a CDR2, and a CDR3 of SEQ ID NOs: 6, 7, and 8, respectively, with collectively a total of one or more amino acid positions in SEQ ID NOs: 6, 7, and 8 substituted with a histidine; (i) an HCVD including a CDR1, a CDR2, and a CDR3 of SEQ ID NOs: 131, 132, and 133, respectively, with collectively a total of one or more amino acid positions in SEQ ID NOs: 131, 132, and 133 substituted with a histidine; and/or an LCVD including a CDR1, a CDR2, and a CDR3 of SEQ ID NOs: 134, 135, and 136, respectively, with collectively a total of one or more amino acid positions in SEQ ID NOs: 134, 135, and 136 substituted with a histidine; and (j) an HCVD including a CDR1, a CDR2, and a CDR3 of SEQ ID NOs: 231, 232, and 233, respectively, with collectively a total of one or more amino acid positions in SEQ ID NOs: 231, 232, and 233 substituted with a histidine; and/or an LCVD including a CDR1, a CDR2, and a CDR3 of SEQ ID NOs: 234, 235, and 236, respectively, optionally with collectively a total of one or more amino acid positions in SEQ ID NOs: 234, 235, and 236 substituted with a histidine.

In some embodiments, the first ABD includes one of: (a) an HCVD that is at least 90% identical to SEQ ID NO: 301, where the HCVD optionally includes a histidine at one or more positions in SEQ ID NO: 301 selected from: 29, 30, 32, 50-54, 58, 60, 96, and 101; (b) an HCVD that is at least 90% identical to SEQ ID NO: 375, where the HCVD optionally includes a histidine at one or more positions in SEQ ID NO: 375 selected from: 26, 27, 30, 32, 34, 51, 52, 55, 58, 59, 63, 97, 99, and 108; (c) an HCVD that is at least 90% identical to SEQ ID NO: 459, where the HCVD optionally includes a histidine at one or more positions in SEQ ID NO: 459 selected from: 32, 99, 101, 102, 106 and 111; (d) an HCVD that is at least 90% identical to SEQ ID NO: 542, where the HCVD optionally includes a histidine at one or more positions in SEQ ID NO: 542 selected from: 27, 29, 30, 31, 32, 53, 55, 58, 102, 104 and 105; (e) an HCVD that is at least 90% identical to SEQ ID NO: 628, where the HCVD optionally includes a histidine at one or more positions in SEQ ID NO: 628 selected from: 26, 28, 30, 50, 52, 53, 54, 55, 56, 57, 100, 101, 102 and 103; (f) an HCVD that is at least 90% identical to SEQ ID NO: 710, where the HCVD optionally includes a histidine at one or more positions in SEQ ID NO: 710 selected from: 29, 30, 31, 33, 35, 50, 53, 57, 58, 65, 96, 97, 98, 100, 101, 103, 105, 107, 108, 109, 110 and 113; and (g) an HCVD that is at least 90% identical to SEQ ID NO: 803, where the HCVD optionally includes a histidine at one or more positions in SEQ ID NO: 803 selected from: 33, 50, 53, 55-58, 64, 100, and 107.

In some embodiments, the first ABD includes one of: (a) an LCVD that is at least 90% identical to SEQ ID NO: 302, where the LCVD optionally includes a histidine at one or more positions in SEQ ID NO: 302 selected from: 25, 30-33, 35, 55-57, 91, 94-96, and 98; (b) an LCVD that is at least 90% identical to SEQ ID NO: 376, where the LCVD optionally includes a histidine at one or more positions in SEQ ID NO: 376 selected from: 25, 29, 30, 31, 32, 33, 50, 51, 52, 53, 89, 91, 92, 93, 96, 97, 100 and 102; (c) an LCVD that is at least 90% identical to SEQ ID NO: 460, where the LCVD optionally includes a histidine at one or more positions in SEQ ID NO: 460 selected from: 30, 31, 33, 51, 92 and 95; (d) an LCVD that is at least 90% identical to SEQ ID NO: 543, where the LCVD optionally includes a histidine at one or more positions in SEQ ID NO: 543 selected from: 25, 29, 30, 31, 32, 89, 92 and 98; (e) an LCVD that is at least 90% identical to SEQ ID NO: 629, where the LCVD optionally includes a histidine at one or more positions in SEQ ID NO: 629 selected from: 25, 26, 28, 29, 33, 51, 55, 89, 91, 95, 96, 97, 98, 99 and 100; (f) an LCVD that is at least 90% identical to SEQ ID NO: 711, where the LCVD optionally includes a histidine at one or more positions in SEQ ID NO: 711 selected from: 25, 27, 29, 30, 35, 36, 56, 57, 91, 93, 94, 95, 98 and 99; and (g) an LCVD that is at least 90% identical to SEQ ID NO: 804, where the LCVD optionally includes a histidine at one or more positions in SEQ ID NO: 804 selected from: 31, 33-35, 52, 54, 98, and 99.

In some embodiments, the first ABD includes one of: (a) an HCVD that is at least 90% identical to SEQ ID NO: 301, where the HCVD optionally includes a histidine at two or more positions in SEQ ID NO: 301 selected from: 29, 30, 32, 50-54, 58, 60, 96, and 101; (b) an HCVD that is at least 90% identical to SEQ ID NO: 375, where the HCVD optionally includes a histidine at two or more positions in SEQ ID NO: 375 selected from: 26, 27, 30, 32, 34, 51, 52, 55, 58, 59, 63, 97, 99, and 108; (c) an HCVD that is at least 90% identical to SEQ ID NO: 459, where the HCVD optionally includes a histidine at two or more positions in SEQ ID NO: 459 selected from: 32, 99, 101, 102, 106 and 111; (d) an HCVD that is at least 90% identical to SEQ ID NO: 542, where the HCVD optionally includes a histidine at two or more positions in SEQ ID NO: 542 selected from: 27, 29, 30, 31, 32, 53, 55, 58, 102, 104 and 105; (e) an HCVD that is at least 90% identical to SEQ ID NO: 628, where the HCVD optionally includes a histidine at two or more positions in SEQ ID NO: 628 selected from: 26, 28, 30, 50, 52, 53, 54, 55, 56, 57, 100, 101, 102 and 103; (f) an HCVD that is at least 90% identical to SEQ ID NO: 710, where the HCVD optionally includes a histidine at two or more positions in SEQ ID NO: 710 selected from: 29, 30, 31, 33, 35, 50, 53, 57, 58, 65, 96, 97, 98, 100, 101, 103, 105, 107, 108, 109, 110 and 113; and (g) an HCVD that is at least 90% identical to SEQ ID NO: 803, where the HCVD optionally includes a histidine at two or more positions in SEQ ID NO: 803 selected from: 33, 50, 53, 55-58, 64, 100, and 107.

In some embodiments, the first ABD includes one of: (a) an LCVD that is at least 90% identical to SEQ ID NO: 302, where the LCVD optionally includes a histidine at two or more positions in SEQ ID NO: 302 selected from: 25, 30-33, 35, 55-57, 91, 94-96, and 98; (b) an LCVD that is at least 90% identical to SEQ ID NO: 376, where the LCVD optionally includes a histidine at two or more positions in SEQ ID NO: 376 selected from: 25, 29, 30, 31, 32, 33, 50, 51, 52, 53, 89, 91, 92, 93, 96, 97, 100 and 102; (c) an LCVD that is at least 90% identical to SEQ ID NO: 460, where the LCVD optionally includes a histidine at two or more positions in SEQ ID NO: 460 selected from: 30, 31, 33, 51, 92 and 95; (d) an LCVD that is at least 90% identical to SEQ ID NO: 543, where the LCVD optionally includes a histidine at two or more positions in SEQ ID NO: 543 selected from: 25, 29, 30, 31, 32, 89, 92 and 98; (e) an LCVD that is at least 90% identical to SEQ ID NO: 629, where the LCVD optionally includes a histidine at two or more positions in SEQ ID NO: 629 selected from: 25, 26, 28, 29, 33, 51, 55, 89, 91, 95, 96, 97, 98, 99 and 100; (f) an LCVD that is at least 90% identical to SEQ ID NO: 711, where the LCVD optionally includes a histidine at two or more positions in SEQ ID NO: 711 selected from: 25, 27, 29, 30, 35, 36, 56, 57, 91, 93, 94, 95, 98 and 99; and (g) an LCVD that is at least 90% identical to SEQ ID NO: 804, where the LCVD optionally includes a histidine at two or more positions in SEQ ID NO: 804 selected from: 31, 33-35, 52, 54, 98, and 99.

In some embodiments, the first ABD includes one of: (a) an HCVD that is at least 90% identical to SEQ ID NO: 301, where the HCVD optionally includes a histidine at one or more positions in SEQ ID NO: 301 selected from: 29, 30, 32, 50-54, 58, 60, 96, and 101; and/or an LCVD that is at least 90% identical to SEQ ID NO: 302, where the LCVD optionally includes a histidine at one or more positions in SEQ ID NO: 302 selected from: 25, 30-33, 35, 55-57, 91, 94-96, and 98; (b) an HCVD that is at least 90% identical to SEQ ID NO: 375, where the HCVD optionally includes a histidine at one or more positions in SEQ ID NO: 375 selected from: 26, 27, 30, 32, 34, 51, 52, 55, 58, 59, 63, 97, 99, and 108; and/or an LCVD that is at least 90% identical to SEQ ID NO: 376, where the LCVD optionally includes a histidine at one or more positions in SEQ ID NO: 376 selected from: 25, 29, 30, 31, 32, 33, 50, 51, 52, 53, 89, 91, 92, 93, 96, 97, 100 and 102; (c) an HCVD that is at least 90% identical to SEQ ID NO: 459, wherein the HCVD optionally includes a histidine at one or more positions in SEQ ID NO: 459 selected from: 32, 99, 101, 102, 106 and 111; and/or an LCVD that is at least 90% identical to SEQ ID NO: 460, where the LCVD optionally includes a histidine at one or more positions in SEQ ID NO: 460 selected from 30, 31, 33, 51, 92 and 95; (d) an HCVD that is at least 90% identical to SEQ ID NO: 542, wherein the HCVD optionally includes a histidine at one or more positions in SEQ ID NO: 542 selected from: 27, 29, 30, 31, 32, 53, 55, 58, 102, 104 and 105; and/or an LCVD that is at least 90% identical to SEQ ID NO: 543, where the LCVD optionally includes a histidine at one or more positions in SEQ ID NO: 543 selected from 25, 29, 30, 31, 32, 89, 92 and 98; (e) an HCVD that is at least 90% identical to SEQ ID NO: 628, where the HCVD optionally includes a histidine at one or more positions in SEQ ID NO: 628 selected from: 26, 28, 30, 50, 52, 53, 54, 55, 56, 57, 100, 101, 102 and 103; and/or an LCVD that is at least 90% identical to SEQ ID NO: 629, where the LCVD optionally includes a histidine at one or more positions in SEQ ID NO: 629 selected from: 25, 26, 28, 29, 33, 51, 55, 89, 91, 95, 96, 97, 98, 99 and 100; (f) an HCVD that is at least 90% identical to SEQ ID NO: 710, wherein the HCVD optionally includes a histidine at one or more positions in SEQ ID NO: 710 selected from: 29, 30, 31, 33, 35, 50, 53, 57, 58, 65, 96, 97, 98, 100, 101, 103, 105, 107, 108, 109, 110 and 113; and/or an LCVD that is at least 90% identical to SEQ ID NO: 711, where the LCVD optionally includes a histidine at one or more positions in SEQ ID NO: 711 selected from 25, 27, 29, 30, 35, 36, 56, 57, 91, 93, 94, 95, 98 and 99; (g) an HCVD that is at least 90% identical to SEQ ID NO: 803, wherein the HCVD optionally includes a histidine at one or more positions in SEQ ID NO: 803 selected from: 33, 50, 53, 55-58, 64, 100, and 107; and/or an LCVD that is at least 90% identical to SEQ ID NO: 804, where the LCVD optionally includes a histidine at one or more positions in SEQ ID NO: 804 selected from 31, 33-35, 52, 54, 98, and 99.

In some embodiments, the first ABD includes one of: (a) an HCVD of SEQ ID NO: 301, or one of 309-342; (b) an HCVD of SEQ ID NO: 375, or one of 383-423; (c) an HCVD of SEQ ID NO: 459, or one of 467-511, (d) an HCVD of SEQ ID NO: 542, or one of 550-592; (e) an HCVD of SEQ ID NO: 628, or one of 636-675; (f) an HCVD of SEQ ID NO: 710, or one of 718-767; and (g) an HCVD of SEQ ID NO: 803, or one of 811-854; and/or the first ABD includes one of: (a) an LCVD of SEQ ID NO: 302, or one of 343-374; (b) an LCVD of SEQ ID NO: 376, or one of 424-458; (c) an LCVD of SEQ ID NO: 460, or one of 512-541; (d) an LCVD of SEQ ID NO: 543, or one of 593-627 (e) an LCVD of SEQ ID NO: 629, or one of 676-709; (f) an LCVD of SEQ ID NO: 711, or one of 768-802; and (g) an LCVD of SEQ ID NO: 804, or one of 855-892.

In some embodiments, the first ABD includes one of:
(a) an HCVD of SEQ ID NO: 301, or one of 309-342; and/or an LCVD of SEQ ID NO: 302, or one of 343-374, where the first ABD does not include (i) an HCVD of SEQ ID NO: 301 and an LCVD of SEQ ID NO: 302; (ii) an HCVD of SEQ ID NO: 301 and an LCVD that is not one of SEQ ID NO: 344, 349-352, 354, 359-361, 363, 366-368, 370, and 373; or (iii) an LCVD of SEQ ID NO: 302 and an HCVD that is not one of SEQ ID NO: 312, 313, 315, 319-323, 327, 329, 335, 340 and 341;
(b) an HCVD of SEQ ID NO: 375, or one of 383-423; and/or an LCVD of SEQ ID NO: 376, or one of 424-458, where the first ABD does not include (i) an HCVD of SEQ ID NO: 375 and an LCVD of SEQ ID NO: 376; (ii) an HCVD of SEQ ID NO: 375 and an LCVD that is not one of SEQ ID NO: 425, 429, 430, 431, 432, 433, 435, 436, 437, 438, 442, 444, 445, 446, 449, 450, 453, and 455; or (iii) an LCVD of SEQ ID NO: 376 and an HCVD that is not one of SEQ ID NO: 383, 384, 387, 389, 391, 394, 395, 398, 401, 402, 406, 410, 412, and 421;
(c) an HCVD of SEQ ID NO: 459, or one of 467-511; and/or an LCVD of SEQ ID NO: 460, or one of 512-541, where the first ABD does not include (i) an HCVD of SEQ ID NO: 459 and an LCVD of SEQ ID NO: 460; (ii) an HCVD of SEQ ID NO: 459 and an LCVD that is not one of SEQ ID NO: 518, 519, 521, 524, 533, and 536; or (iii) an LCVD of SEQ ID NO: 460 and an HCVD that is not one of SEQ ID NO: 473, 496, 498, 499, 503, and 508;
(d) an HCVD of SEQ ID NO: 542, or one of 550-592; and/or an LCVD of SEQ ID NO: 543, or one of 593-627, where the first ABD does not include (i) an HCVD of SEQ ID NO: 542 and an LCVD of SEQ ID NO: 543; (ii) an HCVD of SEQ ID NO: 542 and an LCVD that is not one of SEQ ID NO: 594, 598, 599, 600, 601, 611, 614, and 620; or (iii) an LCVD of SEQ ID NO: 543 and an HCVD that is not one of SEQ ID NO: 551, 553, 554, 555, 556, 563, 565, 568, 582, 584, and 585;
(e) an HCVD of SEQ ID NO: 628, or one of 636-675; and/or an LCVD of SEQ ID NO: 629, or one of 676-709, where the first ABD does not include (i) an HCVD of SEQ ID NO: 628 and an LCVD of SEQ ID NO: 629; (ii) an HCVD of SEQ ID NO: 628 and an LCVD that is not one of SEQ ID NO: 677, 678, 680, 681, 685, 688, 692, 694, 696, 700, 701, 702, 703, 704, and 705; or (iii) an LCVD of SEQ ID NO: 629 and an HCVD that is not one of SEQ ID NO: 636, 638, 640, 646, 648, 649, 650, 651, 652, 653, 666, 667, 668, and 669;
(f) an HCVD of SEQ ID NO: 710, or one of 718-767; and/or an LCVD of SEQ ID NO: 711, or one of 768-802, where the first ABD does not include (i) an HCVD of SEQ ID NO: 710 and an LCVD of SEQ ID NO: 711; (ii) an HCVD of SEQ ID NO: 710 and an LCVD that is not one of SEQ ID NO: 769, 771, 773, 774, 779, 780, 785, 786, 788, 790, 791, 792, 795, and 796; or (iii) an LCVD of SEQ ID NO: 711 and an HCVD that is not one of SEQ ID NO: 721, 722, 723, 725, 727, 728, 731, 735, 736, 743, 744, 745, 746, 748, 749, 751, 753, 755, 756, 757, 758, and 761; and
(g) an HCVD of SEQ ID NO: 803, or one of 811-854; and/or an LCVD of SEQ ID NO: 804, or one of 855-892, where the first ABD does not include (i) an HCVD of SEQ ID NO: 803 and an LCVD of SEQ ID NO: 804; (ii) an HCVD of SEQ ID NO: 803 and an LCVD that is not one of SEQ ID NO: 862, 864-866, 868, 870, 881, and 882; or (iii) an LCVD of SEQ ID NO: 804 and an HCVD that is not one of SEQ ID NO: 818, 821, 824, 826-829, 835, 841, 848, and 850.

In some embodiments, the first antigen-binding domain (ABD) includes an HCVD of one of: (a) an HCVD that is at least 90% identical to SEQ ID NO: 1, where the HCVD includes a histidine at one or more positions in SEQ ID NO: 1 selected from the group consisting of: 24, 27, 28, 29, 30, 31, 32, 34, 53, 54, 55, 57, 58, 64, 98, 100, 102, 103, and 107; (b) an HCVD that is at least 90% identical to SEQ ID NO: 126, where the HCVD includes a histidine at one or more positions in SEQ ID NO: 126 selected from the group consisting of: 53, 54, 56, 57, 60, 102, 103, 104, 105, 106, 108, 109, 110, and 111; and (c) an HCVD that is at least 90% identical to SEQ ID NO: 229, where the HCVD includes a histidine at one or more positions in SEQ ID NO: 229 selected from the group consisting of: 27, 32, 33, 34, 35, 51, 54, 56, 58, and 100. In some embodiments, the first ABD includes an LCVD of one of: (a) an LCVD that is at least 90% identical to SEQ ID NO: 2, where the LCVD includes a histidine at one or more positions in SEQ ID NO: 2 selected from the group consisting of: 25, 29, 31, 35, 60, 93, and 94; (b) an LCVD that is at least 90% identical to SEQ ID NO: 127, where the LCVD includes a histidine at one or more positions in SEQ ID NO: 127 selected from the group consisting of: 25, 26, 28, 29, 31, 32, 33, 50, 90, 92, and 94; and (c) an LCVD that is at least 90% identical to SEQ ID NO: 230, where the LCVD includes a histidine at one or more positions in SEQ ID NO: 230 selected from the group consisting of: 30, 31, 33, 51, 53, 57, 91, 92, 94, and 99.

In some embodiments, the first ABD includes an HCVD of one of: (a) an HCVD that is at least 90% identical to SEQ ID NO: 1, where the HCVD includes a histidine at two or more positions in SEQ ID NO: 1 selected from the group consisting of: 27, 29, 31, 34, and 53; and (b) an HCVD that is at least 90% identical to SEQ ID NO: 126, where the HCVD includes a histidine at two or more positions in SEQ ID NO: 126 selected from the group consisting of: 53, 56, 57, 102, and 105. In some embodiments, the first ABD includes an LCVD of one of: (a) an LCVD that is at least 90% identical to SEQ ID NO: 2, where the LCVD includes a histidine at two or more positions in SEQ ID NO: 2 selected from the group consisting of: 31, 60, and 93; and (b) an LCVD that is at least 90% identical to SEQ ID NO: 127, where the LCVD includes a histidine at two or more positions in SEQ ID NO: 127 from the group consisting of: 28 and 90.

In some embodiments, the first ABD includes one of: (a) an HCVD that is at least 90% identical to SEQ ID NO: 1, where the HCVD includes a histidine at one or more positions in SEQ ID NO: 1 selected from the group consisting of: 24, 27, 28, 29, 30, 31, 32, 34, 53, 54, 55, 57, 58, 64, 98, 100, 102, 103, and 107; and/or an LCVD that is at least 90% identical to SEQ ID NO: 2, where the LCVD includes a histidine at one or more positions in SEQ ID NO: 2 selected from the group consisting of: 25, 29, 31, 35, 60, 93, or 94; (b) an HCVD that is at least 90% identical to SEQ ID NO: 126, where the HCVD includes a histidine at one or more positions in SEQ ID NO: 126 selected from the group consisting of: 53, 54, 56, 57, 60, 102, 103, 104, 105, 106, 108, 109, 110, or 111; and/or an LCVD that is at least 90% identical to SEQ ID NO: 127, where the LCVD includes a histidine at one or more positions in SEQ ID NO: 127 selected from the group consisting of: 25, 26, 28, 29, 31, 32, 33, 50, 90, 92, and 94; and (c) an HCVD that is at least 90% identical to SEQ ID NO: 229, where the HCVD includes a histidine at one or more positions in SEQ ID NO: 229 selected from the group consisting of: 27, 32, 33, 34, 35, 51, 54, 56, 58, or 100; and/or an LCVD that is at least 90% identical to SEQ ID NO: 230, where the LCVD includes a histidine at one or more positions in SEQ ID NO: 230 selected from the group consisting of: 30, 31, 33, 51, 53, 57, 91, 92, 94, and 99.

In some embodiments, the first ABD includes an HCVD of one of: (a) an HCVD of SEQ ID NO: 1, 14, 17, 18, 19, 20, 21, 22, 24, 25, 29, 30, 31, 33, 34, 40, 44, 46, 48, 49, 53, 86, 87, 88, 89, 90, 91, 92, 93, 94, 95, 96, 98, 99, 100, 101, 102, 103, 104, or 105; (b) an HCVD of SEQ ID NO: 126, 147, 148, 150, 151, 154, 166, 167, 168, 169, 170, 172, 173, 174, 175, 208, 209, 210, 211, 212, 213, 214, 215, 216, 217, 218, 219, 220, 221, 222, 224, 225, 226, or 227; and (c) an HCVD of SEQ ID NO: 229, 238, 243, 244, 245, 246, 248, 251, 253, 255, or 267.

In some embodiments, the first ABD includes an LCVD of one of: (a) an LCVD of SEQ ID NO: 2, 56, 60, 62, 66, 69, 76, 77, 78, 110, 113, 114, 119, 122, 123, 124, or 125; (b) an LCVD of SEQ ID NO: 127, 181, 182, 184, 185, 187, 188, 189, 191, 199, 201, 203, or 228; and (c) an LCVD of SEQ ID NO: 230, 275, 276, 278, 281, 283, 287, 289, 290, 292, or 297.

In some embodiments, the first ABD includes one of: (a) an HCVD of SEQ ID NO: 14, 17, 18, 19, 20, 21, 22, 24, 25, 29, 30, 31, 33, 34, 40, 44, 46, 48, 49, 53, 86, 87, 88, 89, 90, 91, 92, 93, 94, 95, 96, 98, 99, 100, 101, 102, 103, 104, or 105, and/or an LCVD of SEQ ID NO: 2, 56, 60, 62, 66, 69, 76, 77, 78, 110, 113, 114, 119, 122, 123, 124, or 125, where the first antigen binding domain does not include (i) an HCVD of SEQ ID NO: 1 and an LCVD of SEQ ID NO: 2; (ii) an HCVD of SEQ ID NO: 1 and LCVD that is not one of SEQ ID NOs: SEQ ID NOs: 56, 60, 62, 66, 69, 76, 77, 78, 110, 113, 114, 119, and 122-125; or (iii) an LCVD of SEQ ID NO: 2 and an HCVD that is not one of SEQ ID NOs: 14, 17-22, 24, 25, 29-31, 33, 34, 40, 44, 46, 48, 49, 53, 86-96, and 98-105; (b) an HCVD of SEQ ID NO: 147, 148, 150, 151, 154, 166, 167, 168, 169, 170, 172, 173, 174, 175, 208, 209, 210, 211, 212, 213, 214, 215, 216, 217, 218, 219, 220, 221, 222, 224, 225, 226, or 227; and/or an LCVD of SEQ ID NO: 127, 181, 182, 184, 185, 187, 188, 189, 191, 199, 201, 203, or 228, where the first ABD does not include (i) an HCVD of SEQ ID NO: 126 and an LCVD of SEQ ID NO: 127; (ii) an HCVD of SEQ ID NO: 126 and an LCVD that is not one of SEQ ID NOs: 181, 182, 184, 185, 187-189, 191, 199, 201, 203, and 228; (iii) an LCVD of SEQ ID NO: 127 and an HCVD that is not one of SEQ ID NOs: 147, 148, 150, 151, 154, 166-170, 172-175, 208-222, and 224-227; and (c) an HCVD of SEQ ID NO: 238, 243, 244, 245, 246, 248, 251, 253, 255, or 267; and/or an LCVD of SEQ ID NO: 230, 275, 276, 278, 281, 283, 287, 289, 290, 292, or 297, where the first ABD does not include (i) an HCVD of SEQ ID NO: 229 and an LCVD of SEQ ID NO: 230; (ii) an HCVD of SEQ ID NO: 229 and an LCVD that is not one of SEQ ID NOs: 275, 276, 278, 281, 283, 287, 289, 290, 292, and 297; and (iii) an LCVD of SEQ ID NO: 230 and an HCVD that is not one of SEQ ID NOs: 238, 243-246, 248, 251, 253, 255, and 267.

In some embodiments, the ABPC is degraded in the target mammalian cell following internalization of the ABPC by the target mammalian cell. In some embodiments, the ABPC includes a conjugated toxin, radioisotope, drug, or small molecule.

In some embodiments, the composition provides for an increase in toxin liberation in the target mammalian cell as compared to a composition including the same amount of a control ABPC. In some embodiments, the composition provides for at least a 20% increase in toxin liberation in the target mammalian cell as compared to a composition including the same amount of a control ABPC. In some embodiments, the composition provides for at least a 50% increase in toxin liberation in the target mammalian cell as compared to a composition including the same amount of a control ABPC. In some embodiments, the composition provides for at least a 2-fold increase in toxin liberation in the target mammalian cell as compared to a composition including the same amount of a control ABPC. In some embodiments, the composition provides for at least a 5-fold increase in toxin liberation in the target mammalian cell as compared to a composition including the same amount of a control ABPC.

In some embodiments, the composition provides for an increase in target mammalian cell killing as compared to a composition including the same amount of a control ABPC. In some embodiments, the composition provides for at least a 20% r 50% increase in target mammalian cell killing as compared to a composition including the same amount of a control ABPC. In some embodiments, the composition provides for at least a 2- or 5-fold increase in target mammalian cell killing as compared to a composition including the same amount of a control ABPC.

In some embodiments, the composition provides for an increase in endolysosomal delivery in the target mammalian cell as compared to a composition including the same amount of a control ABPC. In some embodiments, the composition provides for at least a 20% r 50% increase in endolysosomal delivery in the target mammalian cell as compared to a composition including the same amount of a control ABPC. In some embodiments, the composition provides for at least a 2- or 5-fold increase in endolysosomal delivery in the target mammalian cell as compared to a composition including the same amount of a control ABPC.

In some embodiments, the composition results in a less of a reduction in the level of PTK7 presented on the surface of the target mammalian cell as compared to a composition including the same amount of a control ABPC. In some embodiments, the composition does not result in a detectable reduction in the level of the PTK7 presented on the surface of the target mammalian cell.

Also provided herein are pharmaceutical compositions including an effective amount of an antigen-binding protein construct (ABPC) including: a first ABD that is capable of specifically binding PTK7 or an epitope of PTK7 presented on the surface of a target mammalian cell; and a conjugated toxin, radioisotope, drug, or small molecule, where: (a) the dissociation rate of the first ABD at a pH of about 4.0 to about 6.5 is faster than the dissociation rate at a pH of about 7.0 to about 8.0; or the dissociation constant (K_{D}) of the first ABD at a pH of about 4.0 to about 6.5 is greater than the K_{D} at a pH of about 7.0 to about 8.0; and (b) the composition provides for one or more of: an increase in toxin liberation in the target mammalian cell as compared to a composition including the same amount of a control ABPC; an increase in target mammalian cell killing as compared to a composition including the same amount of a control ABPC; and an increase in endolysosomal delivery in the target mammalian cell as compared to a composition including the same amount of a control ABPC.

In some embodiments, the first ABD includes an HCVD of one of: (a) an HCVD of MYT9345; (b) an HCVD of MYT9359; (c) an HCVD of MYT9361; (d) an HCVD of MYT9412, (e) an HCVD of MYT9460; (f) an HCVD of MYT9792; and (g) an HCVD of MYT9797, each HCVD optionally with one or more histidine substitution. In some embodiments, the first ABD includes an LCVD of one of: (a) an LCVD of MYT9345; (b) an LCVD of MYT9359; (c) an LCVD of MYT9361; (d) an LCVD of MYT9412, (e) an LCVD of MYT9460; (f) an LCVD of MYT9792; and (g) an LCVD of MYT9797, each LCVD optionally with one or more histidine substitution.

In some embodiments, the first ABD includes one of: (a) an HCVD of MYT9345 and/or an LCVD of MYT9345; (b) an HCVD of MYT9359 and/or an LCVD of MYT9359; (c) an HCVD of MYT9361 and/or an LCVD of MYT9361; (d) an HCVD of MYT9412 and/or an LCVD of MYT9412; (e) an HCVD of MYT9460 and/or an LCVD of MYT9460; (f) an HCVD of MYT9792 and/or an LCVD of MYT9792; and (g) an HCVD of MYT9797 and/or an LCVD of MYT9797, each HCVD and/or LCVD optionally with one or more histidine substitution.

In some embodiments, the HCVD includes one of: (a) an HCVD of MYT9345 including SEQ ID NO: 301; (b) an HCVD of MYT9359 including SEQ ID NO: 375; (c) an HCVD of MYT9361 including SEQ ID NO: 459; (d) an HCVD of MYT9412 including SEQ ID NO: 542, (e) an HCVD of MYT9460 including SEQ ID NO: 628; (f) an HCVD of MYT9792 including SEQ ID NO: 710; and (g) an HCVD of MYT9797 including SEQ ID NO: 803. In some embodiments, the LCVD includes one of: (a) an LCVD of MYT9345 including SEQ ID NO: 302; (b) an LCVD of MYT9359 including SEQ ID NO: 376; (c) an LCVD of MYT9361 including SEQ ID NO: 460; (d) an LCVD of MYT9412 including SEQ ID NO: 543, (e) an LCVD of MYT9460 including SEQ ID NO: 629; (f) an LCVD of MYT9792 including SEQ ID NO: 711; and (g) an LCVD of MYT9797 including SEQ ID NO: 804.

In some embodiments, the first ABD includes an HCVD including one of: (a) an HCVD including a CDR1, a CDR2, and a CDR3 of SEQ ID NOs: 303, 304, and 305, respectively, with collectively a total of one or more amino acid positions in SEQ ID NOs: 303, 304, and 305 substituted with a histidine; (b) an HCVD including a CDR1, a CDR2, and a CDR3 of SEQ ID NOs: 377, 378, and 379, respectively, with collectively a total of one or more amino acid positions in SEQ ID NOs: 377, 378, and 379 optionally substituted with a histidine; (c) an HCVD including a CDR1, a CDR2, and a CDR3 of SEQ ID NOs: 461, 462, and 463, respectively, with collectively a total of one or more amino acid positions in SEQ ID NOs: 461, 462, and 463 substituted with a histidine; (d) an HCVD including a CDR1, a CDR2, and a CDR3 of SEQ ID NOs: 544, 545, and 546, respectively, with collectively a total of one or more amino acid positions in SEQ ID NOs: 544, 545, and 546 optionally substituted with a histidine; (e) an HCVD including a CDR1, a CDR2, and a CDR3 of SEQ ID NOs: 630, 631, and 632, respectively, with collectively a total of one or more amino acid positions in SEQ ID NOs: 630, 631, and 632 optionally substituted with a histidine; (f) an HCVD including a CDR1, a CDR2, and a CDR3 of SEQ ID NOs: 712, 713, and 714, respectively, with collectively a total of one or more amino acid positions in SEQ ID NOs: 712, 713, and 714 substituted with a histidine; and (g) an HCVD including a CDR1, a CDR2, and a CDR3 of SEQ ID NOs: 805, 806, and 807, respectively, with collectively a total of one or more amino acid positions in SEQ ID NOs: 805, 806, and 807 substituted with a histidine.

In some embodiments, the first ABD includes an LCVD of one of: (a) an LCVD including a CDR1, a CDR2, and a CDR3 of SEQ ID NOs: 306, 307, and 308, respectively, with collectively a total of one or more amino acid positions in SEQ ID NOs: 306, 307, and 308 optionally substituted with a histidine; (b) an LCVD including a CDR1, a CDR2, and a CDR3 of SEQ ID NOs: 380, 381, and 382, respectively, with collectively a total of one or more amino acid positions in SEQ ID NOs: 380, 381, and 382 optionally substituted with a histidine; (c) an LCVD including a CDR1, a CDR2, and a CDR3 of SEQ ID NOs: 464, 465, and 466, respectively, with collectively a total of one or more amino acid positions in SEQ ID NOs: 464, 465, and 466 substituted with a histidine; (d) an LCVD including a CDR1, a CDR2, and a CDR3 of SEQ ID NOs: 547, 548, and 549, respectively, with collectively a total of one or more amino acid positions in SEQ ID NOs: 547, 548, and 549 optionally substituted with a histidine; (e) an LCVD including a CDR1, a CDR2, and a CDR3 of SEQ ID NOs: 633, 634, and 635, respectively, with collectively a total of one or more amino acid positions in SEQ ID NOs: 633, 634, and 635 substituted with a histidine; (f) an LCVD including a CDR1, a CDR2, and a CDR3 of SEQ ID NOs: 715, 716, and 717, respectively, with collectively a total of one or more amino acid positions in SEQ ID NOs: 715, 716, and 717 substituted with a histidine; and (g) an LCVD including a CDR1, a CDR2, and a CDR3 of SEQ ID NOs: 808, 809, and 810, respectively, with collectively a total of one or more amino acid positions in SEQ ID NOs: 808, 809, and 810 substituted with a histidine.

In some embodiments, the first ABD includes one of: (a) an HCVD including a CDR1, a CDR2, and a CDR3 of SEQ ID NOs: 303, 304, and 305, respectively, with collectively a total of one or more amino acid positions in SEQ ID NOs: 303, 304, and 305 substituted with a histidine, and/or an LCVD including a CDR1, a CDR2, and a CDR3 of SEQ ID NOs: 306, 307, and 308, respectively, with collectively a total of one or more amino acid positions in SEQ ID NOs: 306, 307, and 308 optionally substituted with a histidine; (b) an HCVD including a CDR1, a CDR2, and a CDR3 of SEQ ID NOs: 377, 378, and 379, respectively, with collectively a total of one or more amino acid positions in SEQ ID NOs: 377, 378, and 379 substituted with a histidine, and/or an LCVD including a CDR1, a CDR2, and a CDR3 of SEQ ID NOs: 380, 381, and 382, respectively, with collectively a total of one or more amino acid positions in SEQ ID NOs: 380, 381, and 382 optionally substituted with a histidine; (c) an HCVD including a CDR1, a CDR2, and a CDR3 of SEQ ID NOs: 461, 462, and 463, respectively, with collectively a total of one or more amino acid positions in SEQ ID NOs: 461, 462, and 463 substituted with a histidine, and/or an LCVD including a CDR1, a CDR2, and a CDR3 of SEQ ID NOs: 464, 465, and 466, respectively, with collectively a total of one or more amino acid positions in SEQ ID NOs: 464, 465, and 466 substituted with a histidine; (d) an HCVD including a CDR1, a CDR2, and a CDR3 of SEQ ID NOs: 544, 545, and 546, respectively, with collectively a total of one or more amino acid positions in SEQ ID NOs: 544, 545, and 546 optionally substituted with a histidine; and/or an LCVD including a CDR1, a CDR2, and a CDR3 of SEQ ID NOs: 547, 548, and 549, respectively, with collectively a total of one or more amino acid positions in SEQ ID NOs: 547, 548, and 549 optionally substituted with a histidine; (e) an HCVD including a CDR1, a CDR2, and a CDR3 of SEQ ID NOs: 630, 631, and 632, respectively, with collectively a total of one or more amino acid positions in SEQ ID NOs: 630, 631, and 632 substituted with a histidine, and/or an LCVD including a CDR1, a CDR2, and a CDR3 of SEQ ID NOs: 633, 634, and 635, respectively, with collectively a total of one or more amino acid positions in SEQ ID NOs: 633, 634, and 635 substituted with a histidine; (f) an HCVD including a CDR1, a CDR2, and a CDR3 of SEQ ID NOs: 712, 713, and 714, respectively, with collectively a total of one or more amino acid positions in SEQ ID NOs: 712, 713, and 714 substituted with a histidine, and/or an LCVD including a CDR1, a CDR2, and a CDR3 of SEQ ID NOs: 715, 716, and 717, respectively, with collectively a total of one or more amino acid positions in SEQ ID NOs: 715, 716, and 717 substituted with a histidine; and (g) an HCVD including a CDR1, a CDR2, and a CDR3 of SEQ ID NOs: 805, 806, and 807, respectively, with collectively a total of one or more amino acid positions in SEQ ID NOs: 805, 806, and 807 optionally substituted with a histidine; and/or an LCVD including a CDR1, a CDR2, and a CDR3 of SEQ ID NOs: 808, 809, and 810, respectively, with collectively a total of one or more amino acid positions in SEQ TD NOs: 808, 809, and 810 substituted with a histidine.

In some embodiments, the first ABD includes an HCVD of one of: (a) an HCVD that is at least 90% identical to SEQ ID NO: 301, where the HCVD optionally includes a histidine at one or more positions in SEQ ID NO: 301 selected from: 29, 30, 32, 50-54, 58, 60, 96, and 101; (b) an HCVD that is at least 90% identical to SEQ ID NO: 375, where the HCVD optionally includes a histidine at one or more positions in SEQ ID NO: 375 selected from: 26, 27, 30, 32, 34, 51, 52, 55, 58, 59, 63, 97, 99, and 108; (c) an HCVD that is at least 90% identical to SEQ ID NO: 459, where the HCVD optionally includes a histidine at one or more positions in SEQ ID NO: 459 selected from: 32, 99, 101, 102, 106 and 111; (d) an HCVD that is at least 90% identical to SEQ ID NO: 542, where the HCVD optionally includes a histidine at one or more positions in SEQ ID NO: 542 selected from: 27, 29, 30, 31, 32, 53, 55, 58, 102, 104 and 105; (e) an HCVD that is at least 90% identical to SEQ ID NO: 628, where the HCVD optionally includes a histidine at one or more positions in SEQ ID NO: 628 selected from: 26, 28, 30, 50, 52, 53, 54, 55, 56, 57, 100, 101, 102 and 103; (f) an HCVD that is at least 90% identical to SEQ ID NO: 710, where the HCVD optionally includes a histidine at one or more positions in SEQ ID NO: 710 selected from: 29, 30, 31, 33, 35, 50, 53, 57, 58, 65, 96, 97, 98, 100, 101, 103, 105, 107, 108, 109, 110 and 113; and (g) an HCVD that is at least 90% identical to SEQ ID NO: 803, where the HCVD optionally includes a histidine at one or more positions in SEQ ID NO: 803 selected from: 33, 50, 53, 55-58, 64, 100, and 107.

In some embodiments, the first ABD includes an LCVD of one of: (a) an LCVD that is at least 90% identical to SEQ ID NO: 302, where the LCVD optionally includes a histidine at one or more positions in SEQ ID NO: 302 selected from: 25, 30-33, 35, 55-57, 91, 94-96, and 98; (b) an LCVD that is at least 90% identical to SEQ ID NO: 376, where the LCVD optionally includes a histidine at one or more positions in SEQ ID NO: 376 selected from: 25, 29, 30, 31, 32, 33, 50, 51, 52, 53, 89, 91, 92, 93, 96, 97, 100 and 102; (c) an LCVD that is at least 90% identical to SEQ ID NO: 460, where the LCVD optionally includes a histidine at one or more positions in SEQ ID NO: 460 selected from 30, 31, 33, 51, 92 and 95; (d) an LCVD that is at least 90% identical to SEQ ID NO: 543, where the LCVD optionally includes a histidine at one or more positions in SEQ ID NO: 543 selected from 25, 29, 30, 31, 32, 89, 92 and 98; (e) an LCVD that is at least 90% identical to SEQ ID NO: 629, where the LCVD optionally includes a histidine at one or more positions in SEQ ID NO: 629 selected from: 25, 26, 28, 29, 33, 51, 55, 89, 91, 95, 96, 97, 98, 99 and 100; (f) an LCVD that is at least 90% identical to SEQ ID NO: 711, where the LCVD optionally includes a histidine at one or more positions in SEQ ID NO: 711 selected from 25, 27, 29, 30, 35, 36, 56, 57, 91, 93, 94, 95, 98 and 99; and (g) an LCVD that is at least 90% identical to SEQ ID NO: 804, where the LCVD optionally includes a histidine at one or more positions in SEQ ID NO: 804 selected from 31, 33-35, 52, 54, 98, and 99.

In some embodiments, the first ABD includes an HCVD of one of: (a) an HCVD that is at least 90% identical to SEQ ID NO: 301, where the HCVD optionally includes a histidine at two or more positions in SEQ ID NO: 301 selected from: 29, 30, 32, 50-54, 58, 60, 96, and 101; (b) an HCVD that is at least 90% identical to SEQ ID NO: 375, where the HCVD optionally includes a histidine at two or more positions in SEQ ID NO: 375 selected from: 26, 27, 30, 32, 34, 51, 52, 55, 58, 59, 63, 97, 99, and 108; (c) an HCVD that is at least 90% identical to SEQ ID NO: 459, where the HCVD optionally includes a histidine at two or more positions in SEQ ID NO: 459 selected from: 32, 99, 101, 102, 106 and 111; (d) an HCVD that is at least 90% identical to SEQ ID NO: 542, where the HCVD optionally includes a histidine at two or more positions in SEQ ID NO: 542 selected from: 27, 29, 30, 31, 32, 53, 55, 58, 102, 104 and 105; (e) an HCVD that is at least 90% identical to SEQ ID NO: 628, where the HCVD optionally includes a histidine at two or more positions in SEQ ID NO: 628 selected from: 26, 28, 30, 50, 52, 53, 54, 55, 56, 57, 100, 101, 102 and 103; (f) an HCVD that is at least 90% identical to SEQ ID NO: 710, where the HCVD optionally includes a histidine at two or more positions in SEQ ID NO: 710 selected from: 29, 30, 31, 33, 35, 50, 53, 57, 58, 65, 96, 97, 98, 100, 101, 103, 105, 107, 108, 109, 110 and 113; and (g) an HCVD that is at least 90% identical to SEQ ID NO: 803, where the HCVD optionally includes a histidine at two or more positions in SEQ ID NO: 803 selected from: 33, 50, 53, 55-58, 64, 100, and 107.

In some embodiments, the first ABD includes an LCVD of one of: (a) an LCVD that is at least 90% identical to SEQ ID NO: 302, where the LCVD optionally includes a histidine at two or more positions in SEQ ID NO: 302 selected from: 25, 30-33, 35, 55-57, 91, 94-96, and 98; (b) an LCVD that is at least 90% identical to SEQ ID NO: 376, where the LCVD optionally includes a histidine at two or more positions in SEQ ID NO: 376 from the group consisting of: 25, 29, 30, 31, 32, 33, 50, 51, 52, 53, 89, 91, 92, 93, 96, 97, 100 and 102; (c) an LCVD that is at least 90% identical to SEQ ID NO: 460, where the LCVD optionally includes a histidine at two or more positions in SEQ ID NO: 460 from the group consisting of: 30, 31, 33, 51, 92 and 95; (d) an LCVD that is at least 90% identical to SEQ ID NO: 543, where the LCVD optionally includes a histidine at two or more positions in SEQ ID NO: 543 from the group consisting of: 25, 29, 30, 31, 32, 89, 92 and 98; (e) an LCVD that is at least 90% identical to SEQ ID NO: 629, where the LCVD optionally includes a histidine at two or more positions in SEQ ID NO: 629 from the group consisting of: 25, 26, 28, 29, 33, 51, 55, 89, 91, 95, 96, 97, 98, 99 and 100; (f) an LCVD that is at least 90% identical to SEQ ID NO: 711, where the LCVD optionally includes a histidine at two or more positions in SEQ ID NO: 711 from the group consisting of: 25, 27, 29, 30, 35, 36, 56, 57, 91, 93, 94, 95, 98 and 99; and (g) an LCVD that is at least 90% identical to SEQ ID NO: 804, where the LCVD optionally includes a histidine at two or more positions in SEQ ID NO: 804 from the group consisting of: 31, 33-35, 52, 54, 98, and 99.

In some embodiments, the first ABD includes one of: (a) an HCVD that is at least 90% identical to SEQ ID NO: 301, where the HCVD optionally includes a histidine at one or more positions in SEQ ID NO: 301 selected from: 29, 30, 32, 50-54, 58, 60, 96, and 101, and/or an LCVD that is at least 90% identical to SEQ ID NO: 302, where the LCVD optionally includes a histidine at one or more positions in SEQ ID NO: 302 selected from: 25, 30-33, 35, 55-57, 91, 94-96, and 98; (b) an HCVD that is at least 90% identical to SEQ ID NO: 375, where the HCVD optionally includes a histidine at one or more positions in SEQ ID NO: 375 selected from: 26, 27, 30, 32, 34, 51, 52, 55, 58, 59, 63, 97, 99, and 108, and/or an LCVD that is at least 90% identical to SEQ ID NO: 376, where the LCVD optionally includes a histidine at one or more positions in SEQ ID NO: 376 selected from: 25, 29, 30, 31, 32, 33, 50, 51, 52, 53, 89, 91, 92, 93, 96, 97, 100 and 102; (c) an HCVD that is at least 90% identical to SEQ ID NO: 459, where the HCVD optionally includes a histidine at one or more positions in SEQ ID NO: 459 selected from: 32, 99, 101, 102, 106 and 111, and/or an LCVD that is at least 90% identical to SEQ ID NO: 460, where the LCVD optionally includes a histidine at one or more positions in SEQ ID NO: 460 selected from 30, 31, 33, 51, 92 and 95; (d) an HCVD that is at least 90% identical to SEQ ID NO: 542, where the HCVD optionally includes a histidine at one or more positions in SEQ ID NO: 542 selected from: 27, 29, 30, 31, 32, 53, 55, 58, 102, 104 and 105, and/or an LCVD that is at least 90% identical to SEQ ID NO: 543, where the LCVD optionally includes a histidine at one or more positions in SEQ ID NO: 543 selected from 25, 29, 30, 31, 32, 89, 92 and 98; (e) an HCVD that is at least 90% identical to SEQ ID NO: 628, where the HCVD optionally includes a histidine at one or more positions in SEQ ID NO: 628 selected from: 26, 28, 30, 50, 52, 53, 54, 55, 56, 57, 100, 101, 102 and 103, and/or an LCVD that is at least 90% identical to SEQ ID NO: 629, where the LCVD optionally includes a histidine at one or more positions in SEQ ID NO: 629 selected from: 25, 26, 28, 29, 33, 51, 55, 89, 91, 95, 96, 97, 98, 99 and 100; (f) an HCVD that is at least 90% identical to SEQ ID NO: 710, where the HCVD optionally includes a histidine at one or more positions in SEQ ID NO: 710 selected from: 29, 30, 31, 33, 35, 50, 53, 57, 58, 65, 96, 97, 98, 100, 101, 103, 105, 107, 108, 109, 110 and 113, and/or an LCVD that is at least 90% identical to SEQ ID NO: 711, where the LCVD optionally includes a histidine at one or more positions in SEQ ID NO: 711 selected from 25, 27, 29, 30, 35, 36, 56, 57, 91, 93, 94, 95, 98 and 99; and (g) an HCVD that is at least 90% identical to SEQ ID NO: 803, where the HCVD optionally includes a histidine at one or more positions in SEQ ID NO: 803 selected from: 33, 50, 53, 55-58, 64, 100, and 107, and/or an LCVD that is at least 90% identical to SEQ ID NO: 804, where the LCVD optionally includes a histidine at one or more positions in SEQ ID NO: 804 selected from 31, 33-35, 52, 54, 98, and 99.

In some embodiments, the first ABD includes one of: (a) an HCVD of SEQ ID NO: 301, or one of 309-342; (b) an HCVD of SEQ ID NO: 375, or one of 383-423; (c) an HCVD of SEQ ID NO: 459, or one of 467-511; (d) an HCVD of SEQ ID NO: 542, or one of 550-592. (e) an HCVD of SEQ ID NO: 628, or one of 636-675; (f) an HCVD of SEQ ID NO: 710, or one of 718-767; and (g) an HCVD of SEQ ID NO: 803, or one of 811-854. In some embodiments, the first ABD includes one of: (a) an LCVD of SEQ ID NO: 302, or one of 343-374; (b) an LCVD of SEQ ID NO: 376, or one of 424-458; (c) an LCVD of SEQ ID NO: 460, or one of 512-541; (d) an LCVD of SEQ ID NO: 543, or one of 593-627; (e) an LCVD of SEQ ID NO: 629, or one of 676-709; (f) an LCVD of SEQ ID NO: 711, or one of 768-802; and (g) an LCVD of SEQ ID NO: 804, or one of 855-892.

In some embodiments, the first ABD includes one of:
(a) an HCVD of any one of SEQ ID NO: 301, or one of 309-342, and/or an LCVD of any one of SEQ ID NO: 302, or one of 343-374, where the first ABD does not include (i) an HCVD of SEQ ID NO: 301 and an LCVD of SEQ ID NO: 302; (ii) an HCVD of SEQ ID NO: 301 and an LCVD that is not one of SEQ ID NO: 344, 349-352, 354, 359-361, 363, 366-368, 370, and 373; or (iii) an HCVD that is not one of SEQ ID NO: 312, 313, 315, 319-323, 327, 329, 335, 340 and 341 and an LCVD of SEQ ID NO: 302;
(b) an HCVD of any one of SEQ ID NO: 375, or one of 383-423, and/or an LCVD of any one of SEQ ID NO: 376, or one of 424-458, where the first ABD does not include (i) an HCVD of SEQ ID NO: 375 and an LCVD of SEQ ID NO: 376; (ii) an HCVD of SEQ ID NO: 375 and an LCVD that is not one of SEQ ID NO: 425, 429, 430, 431, 432, 433, 435, 436, 437, 438, 442, 444, 445, 446, 449, 450, 453, and 455; or (iii) an HCVD that is not one of SEQ ID NO: 383, 384, 387, 389, 391, 394, 395, 398, 401, 402, 406, 410, 412, and 421 and an LCVD of SEQ ID NO: 376;
(c) an HCVD of any one of SEQ ID NO: 459, or one of 467-511, and/or an LCVD of any one of SEQ ID NO: 460, or one of 512-541, where the first ABD does not include (i) an HCVD of SEQ ID NO: 459 and an LCVD of SEQ ID NO: 460; (ii) an HCVD of SEQ ID NO: 459 and an LCVD that is not one of SEQ ID NO: 518, 519, 521, 524, 533, and 536; or (iii) an HCVD that is not one of SEQ ID NO: 473, 496, 498, 499, 503, and 508 and an LCVD of SEQ ID NO: 460;
(d) an HCVD of any one of SEQ ID NO: 542, or one of 550-592, and/or an LCVD of any one of SEQ ID NO: 543, or one of 593-627, where the first ABD does not include (i) an HCVD of SEQ ID NO: 542 and an LCVD of SEQ ID NO: 543; (ii) an HCVD of SEQ ID NO: 542 and an LCVD that is not one of SEQ ID NO: 594, 598, 599, 600, 601, 611, 614, and 620; or (iii) an HCVD that is not one of SEQ ID NO: 551, 553, 554, 555, 556, 563, 565, 568, 582, 584, and 585 and an LCVD of SEQ ID NO: 543;
(e) an HCVD of any one of SEQ ID NO: 628, or one of 636-675, and/or an LCVD of any one of SEQ ID NO: 629, or one of 676-709, where the first ABD does not include (i) an HCVD of SEQ ID NO: 628 and an LCVD of SEQ ID NO: 629; (ii) an HCVD of SEQ ID NO: 628 and an LCVD that is not one of SEQ ID NO: 677, 678, 680, 681, 685, 688, 692, 694, 696, 700, 701, 702, 703, 704, and 705; or (iii) an HCVD that is not one of SEQ ID NO: 636, 638, 640, 646, 648, 649, 650, 651, 652, 653, 666, 667, 668, and 669 and an LCVD of SEQ ID NO: 629;
(f) an HCVD of any one of SEQ ID NO: 710, or one of 718-767, and/or an LCVD of any one of SEQ ID NO: 711, or one of 768-802, where the first ABD does not include (i) an HCVD of SEQ ID NO: 710 and an LCVD of SEQ ID NO: 711; (ii) an HCVD of SEQ ID NO: 710 and an LCVD that is not one of SEQ ID NO: 769, 771, 773, 774, 779, 780, 785, 786, 788, 790, 791, 792, 795, and 796; or (iii) an HCVD that is not one of SEQ ID NO: 721, 722, 723, 725, 727, 728, 731, 735, 736, 743, 744, 745, 746, 748, 749, 751, 753, 755, 756, 757, 758, and 761 and an LCVD of SEQ ID NO: 711; and
(g) an HCVD of any one of SEQ ID NO: 803, or one of 811-854, and/or an LCVD of any one of SEQ ID NO: 804, or one of 855-892, where the first ABD does not include (i) an HCVD of SEQ ID NO: 803 and an LCVD of SEQ ID NO: 804; (ii) an HCVD of SEQ ID NO: 803 and an LCVD that is not one of SEQ ID NO: 862, 864-866, 868, 870, 881, and 882; or (iii) an HCVD 5 that is not one of SEQ ID NO: 818, 821, 824, 826-829, 835, 841, 848, and 850 and an LCVD of SEQ ID NO: 804.

In some embodiments, the composition provides for an increase in toxin liberation in the target mammalian cell as compared to a composition including the same amount of a control ABPC. In some embodiments, the composition provides for at least a 20% r 50% increase in toxin liberation in the target mammalian cell as compared to a composition including the same amount of a control ABPC. In some embodiments, the composition provides for at least a 2- or 5-fold increase in toxin liberation in the target mammalian cell as compared to a composition including the same amount of a control ABPC.

In some embodiments, the composition provides for an increase in target mammalian cell 15 killing as compared to a composition including the same amount of a control ABPC. In some embodiments, the composition provides for at least a 20% r 50% increase in target mammalian cell killing as compared to a composition including the same amount of a control ABPC. In some embodiments, the composition provides for at least a 2- or 5-fold increase in target mammalian cell killing as compared to a composition including the same amount of a control ABPC.

In some embodiments, the composition provides for an increase in endolysosomal delivery, including at least a 20% r 50% increase in endolysosomal delivery as compared to a composition including the same amount of a control ABPC. In some embodiments, the composition provides for at least a 2- or 5-fold increase in endolysosomal delivery in the target mammalian cell as compared to a composition including the same amount of a control ABPC.

In some embodiments, the composition results in a less of a reduction in the level of PTK7 presented on the surface of the target mammalian cell as compared to a composition including the same amount of a control ABPC. In some embodiments, the composition does not result in a detectable reduction in the level of the PTK7 presented on the surface of the target mammalian cell. In some embodiments, the target mammalian cell is a cancer cell.

In some embodiments, the dissociation rate of the ABD at a pH of ~4.0 to ~6.5 is at least 10% faster than the dissociation rate of the ABD at a pH of ~7.0 to ~8.0. In some embodiments, the dissociation rate of the ABD at a pH of ~4.0 to ~6.5 is at least 3-fold faster than the dissociation rate of the ABD at a pH of ~7.0 to ~8.0. In some embodiments, the dissociation rate of the ABD at a pH of ~4.0 to ~6.5 is at least 10-fold faster than the dissociation rate of the ABD at a pH of ~7.0 to ~8.0. In some embodiments, the K_{D} of the ABD at a pH of ~4.0 to ~6.5 is at least 10% greater than the K_{D} of the ABD at a pH of ~7.0 to ~8.0. In some embodiments, the K_{D} of the ABD at a pH of ~4.0 to ~6.5 is at least 3-fold greater than the K_{D} of the ABD at a pH of ~7.0 to ~8.0. In some embodiments, the K_{D} of the ABD at a pH of ~4.0 to ~6.5 is at least 10-fold greater than the K_{D} of the ABD at a pH of ~7.0 to ~8.0.

In some embodiments, the ABPC is cytotoxic or cytostatic to the target mammalian cell.

In some embodiments, the ABPC is cross-reactive with a non-human primate PTK7 and human PTK7. In some embodiments, the ABPC is cross-reactive with a non-human primate PTK7, a human PTK7, and one or both of rat PTK7 and a mouse PTK7. In some embodiments, the ABPC is cross-reactive with a non-human primate PTK7, a human PTK7, a rat PTK7, and a mouse PTK7. In some embodiments, the ABD binds to an epitope of PTK7 that is present on the surface of cells from an Old World Monkey.

In some embodiments, the ABPC includes a single polypeptide. In some embodiments, the ABD is selected from a VH domain, a VHH domain, a VNAR domain, and a scFv. In some embodiments, the ABPC is a BiTe, a (scFv)₂, a nanobody, a nanobody-HSA, a DART, a TandAb, a scDiabody, a scDiabody-CH3, scFv-CH-CL-scFv, a HSAbody, scDiabody-HSA, or a tandem-scFv.

In some embodiments, the ABPC includes two or more polypeptides. In some embodiments, the ABPC is selected from the group of an antibody, a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a K-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, and a DEP conjugate.

In some embodiments, at least one polypeptide of the ABPC is conjugated to the toxin, the radioisotope, the drug, or the small molecule via a cleavable linker. In some embodiments, at least one polypeptide of the ABPC is conjugated to the toxin, the radioisotope, the drug, or the small molecule via a non-cleavable linker.

In some embodiments, the half-life of the ABPC in vivo is increased as compared to the half-life of a control ABPC in vivo. In some embodiments, the half-life of the ABPC in vivo is increased about 5% to about 95% as compared to the half-life of a control ABPC in vivo. In some embodiments, the half-life of the ABPC in vivo is increased about 10% to about 95% as compared to the half-life of a control ABPC in vivo. In some embodiments, the half-life of the ABPC in vivo is increased about 30% to about 95% as compared to the half-life of a control ABPC in vivo. In some embodiments, the half-life of the ABPC in vivo is increased about 50% to about 95% as compared to the half-life of a control ABPC in vivo. In some embodiments, the half-life of the ABPC in vivo is increased about 70% to about 95% as compared to the half-life of a control ABPC in vivo. In other embodiments, the in vivo half-life may be decreased.

In some embodiments, the control ABPC is capable of specifically binding to PTK7 or an epitope of PTK7 presented on the surface of a target mammalian cell, where: (a) the control ABPC includes a first ABD: (b) the dissociation rate of the first ABD of the control ABPC at a pH of ~4.0 to ~6.5 is no more than 3-fold faster than the dissociation rate at a pH of ~7.0 to ~8.0; and (c) the dissociation constant (K_{D}) of the first ABD of the control ABPC at a pH of ~4.0 to ~6.5 is no more than 3-fold greater than the K_{D} at a pH of ~7.0 to ~8.0.

In some embodiments, the control ABPC is capable of specifically binding to PTK7 or an epitope of PTK7 presented on the surface of a target mammalian cell, where: (a) the control ABPC includes a first ABD; (b) the dissociation rate of the first ABD of the control ABPC at a pH of ~4.0 to ~6.5 is no more than 2-fold faster than the dissociation rate at a pH of ~7.0 to ~8.0; and (c) the dissociation constant (K_{D}) of the first ABD of the control ABPC at a pH of ~4.0 to ~6.5 is no more than 2-fold greater than the K_{D} at a pH of ~7.0 to ~8.0.

In some embodiments, the control ABPC is capable of specifically binding to PTK7 or an epitope of PTK7 presented on the surface of a target mammalian cell, where: (a) the control ABPC includes a first ABD; (b) the dissociation rate of the first ABD of the control ABPC at a pH of ~4.0 to ~6.5 is no more than 1-fold faster than the dissociation rate at a pH of ~7.0 to ~8.0; and (c) the dissociation constant (K_{D}) of the first ABD of the control ABPC at a pH of ~4.0 to ~6.5 is no more than 1-fold greater than the K_{D} at a pH of ~7.0 to ~8.0.

In some embodiments, the control ABPC is selected from MYT9345, MYT9359, MYT9361, MYT9412, MYT9460, MYT9792, MYT9797, cofetuzumab, 7C8, and 12C6.

In some embodiments, the ABPC includes a second ABD.

Also provided herein are kits including at least one dose of any of the pharmaceutical compositions described herein.

Also provided herein are antigen-binding protein constructs (ABPCs) including: a first ABD that is capable of specifically binding PTK7 or an epitope of PTK7 presented on the surface of a target mammalian cell, where: (a) the dissociation rate of the first ABD at a pH of about 4.0 to about 6.5 is faster than the dissociation rate at a pH of about 7.0 to about 8.0; or (b) the dissociation constant (K_{D}) of the first ABD at a pH of about 4.0 to about 6.5 is greater than the K_{D} at a pH of about 7.0 to about 8.0.

In some embodiments, the first ABD includes one of: (a) an HCVD of MYT9345; (b) an HCVD of MYT9359; (c) an HCVD of MYT9361; (d) an HCVD of MYT9412; (e) an HCVD of MYT9460; (f) an HCVD of MYT9792; (g) an HCVD of MYT9797; (h) an HCVD of cofetuzumab; (i) an HCVD of 7C8; and (j) an HCVD of 12C6, each HCVD optionally with one or more amino acids substituted with a histidine.

In some embodiments, the first ABD includes one of: (a) an LCVD of MYT9345; (b) an LCVD of MYT9359; (c) an LCVD of MYT9361; (d) an LCVD of MYT9412; (e) an LCVD of MYT9460; (f) an LCVD of MYT9792; (g) an LCVD of MYT9797, (h) an LCVD of cofetuzumab; (i) an LCVD of 7C8; and (j) an LCVD of 12C6, each LCVD optionally with one or more amino acids substituted with a histidine.

In some embodiments, the first ABD includes one of: (a) an HCVD of MYT9345 and/or an LCVD of MYT9345, either or both with one or more amino acids optionally substituted with a histidine; (b) an HCVD of MYT9359 and/or an LCVD of MYT9359, either or both with one or more amino acids optionally substituted with a histidine; (c) an HCVD of MYT9361 and/or an LCVD of MYT9361, either or both with one or more amino acids optionally substituted with a histidine; (d) an HCVD of MYT9412 and/or an LCVD of MYT9412, either or both with one or more amino acids optionally substituted with a histidine; (e) an HCVD of MYT9460 and/or an LCVD of MYT9460, either or both with one or more amino acids optionally substituted with a histidine; (f) an HCVD of MYT9792 and/or an LCVD of MYT9792, either or both with one or more amino acids optionally substituted with a histidine; and (g) an HCVD of MYT9797 and/or an LCVD of MYT9797, either or both with one or more amino acids substituted with a histidine.

In some embodiments, the HCVD includes one of: (a) an HCVD of MYT9345 including SEQ ID NO: 301; (b) an HCVD of MYT9359 including SEQ ID NO: 375; (c) an HCVD of MYT9361 including SEQ ID NO: 459; (d) an HCVD of MYT9412 including SEQ ID NO: 542; (e) an HCVD of MYT9460 including SEQ ID NO: 628; (f) an HCVD of MYT9792 including SEQ ID NO: 710; and (g) an HCVD of MYT9797 including SEQ ID NO: 803.

In some embodiments, the LCVD includes one of: (a) an LCVD of MYT9345 including SEQ ID NO: 302; (b) an LCVD of MYT9359 including SEQ ID NO: 376; (c) an LCVD of MYT9361 including SEQ ID NO: 460; (d) an LCVD of MYT9412 including SEQ ID NO: 543; (e) an LCVD of MYT9460 including SEQ ID NO: 629; (f) an LCVD of MYT9792 including SEQ ID NO: 711; and (g) an LCVD of MYT9797 including SEQ ID NO: 804.

In some embodiments, the first ABD includes an HCVD of one of: (a) an HCVD including a CDR1, a CDR2, and a CDR3 of SEQ ID NOs: 303, 304, and 305, respectively, with collectively a total of one or more amino acid positions in SEQ ID NOs: 303, 304, and 305 substituted with a histidine; (b) an HCVD including a CDR1, a CDR2, and a CDR3 of SEQ ID NOs: 377, 378, and 379, respectively, with collectively a total of one or more amino acid positions in SEQ ID NOs: 377, 378, and 379 optionally substituted with a histidine; (c) an HCVD including a CDR1, a CDR2, and a CDR3 of SEQ ID NOs: 461, 462, and 463, respectively, with collectively a total of one or more amino acid positions in SEQ ID NOs: 461, 462, and 463 substituted with a histidine; (d) an HCVD including a CDR1, a CDR2, and a CDR3 of SEQ ID NOs: 544, 545, and 546, respectively, with collectively a total of one or more amino acid positions in SEQ ID NOs: 544, 545, and 546 optionally substituted with a histidine; (e) an HCVD including a CDR1, a CDR2, and a CDR3 of SEQ ID NOs: 630, 631, and 632, respectively, with collectively a total of one or more amino acid positions in SEQ ID NOs: 630, 631, and 632 optionally substituted with a histidine; (f) an HCVD including a CDR1, a CDR2, and a CDR3 of SEQ ID NOs: 712, 713, and 714, respectively, with collectively a total of one or more amino acid positions in SEQ ID NOs: 712, 713, and 714 substituted with a histidine; and (g) an HCVD including a CDR1, a CDR2, and a CDR3 of SEQ ID NOs: 805, 806, and 807, respectively, with collectively a total of one or more amino acid positions in SEQ TD NOs: 805, 806, and 807 substituted with a histidine.

In some embodiments, the first ABD includes an LCVD of one of: (a) an LCVD including a CDR1, a CDR2, and a CDR3 of SEQ ID NOs: 306, 307, and 308, respectively, with collectively a total of one or more amino acid positions in SEQ ID NOs: 306, 307, and 308 optionally substituted with a histidine; (b) an LCVD including a CDR1, a CDR2, and a CDR3 of SEQ ID NOs: 380, 381, and 382, respectively, with collectively a total of one or more amino acid positions in SEQ ID NOs: 380, 381, and 382 optionally substituted with a histidine; (c) an LCVD including a CDR1, a CDR2, and a CDR3 of SEQ ID NOs: 464, 465, and 466, respectively, with collectively a total of one or more amino acid positions in SEQ ID NOs: 464, 465, and 466 substituted with a histidine; (d) an LCVD including a CDR1, a CDR2, and a CDR3 of SEQ ID NOs: 547, 548, and 549, respectively, with collectively a total of one or more amino acid positions in SEQ ID NOs: 547, 548, and 549 optionally substituted with a histidine; (e) an LCVD including a CDR1, a CDR2, and a CDR3 of SEQ ID NOs: 633, 634, and 635, respectively, with collectively a total of one or more amino acid positions in SEQ ID NOs: 633, 634, and 635 substituted with a histidine; (f) an LCVD including a CDR1, a CDR2, and a CDR3 of SEQ ID NOs: 715, 716, and 717, respectively, with collectively a total of one or more amino acid positions in SEQ ID NOs: 715, 716, and 717 substituted with a histidine; and (g) an LCVD including a CDR1, a CDR2, and a CDR3 of SEQ ID NOs: 808, 809, and 810, respectively, with collectively a total of one or more amino acid positions in SEQ ID NOs: 808, 809, and 810 substituted with a histidine.

In some embodiments, the first ABD includes one of: (a) an HCVD including a CDR1, a CDR2, and a CDR3 of SEQ ID NOs: 303, 304, and 305, respectively, with collectively a total of one or more amino acid positions in SEQ ID NOs: 303, 304, and 305 substituted with a histidine; and/or an LCVD including a CDR1, a CDR2, and a CDR3 of SEQ ID NOs: 306, 307, and 308, respectively, with collectively a total of one or more amino acid positions in SEQ ID NOs: 306, 307, and 308 optionally substituted with a histidine; (b) an HCVD including a CDR1, a CDR2, and a CDR3 of SEQ ID NOs: 377, 378, and 379, respectively, with collectively a total of one or more amino acid positions in SEQ ID NOs: 377, 378, and 379 optionally substituted with a histidine; and/or an LCVD including a CDR1, a CDR2, and a CDR3 of SEQ ID NOs: 380, 381, and 382, respectively, with collectively a total of one or more amino acid positions in SEQ ID NOs: 380, 381, and 382 optionally substituted with a histidine; (c) an HCVD including a CDR1, a CDR2, and a CDR3 of SEQ ID NOs: 461, 462, and 463, respectively, with collectively a total of one or more amino acid positions in SEQ ID NOs: 461, 462, and 463 substituted with a histidine; and/or an LCVD including a CDR1, a CDR2, and a CDR3 of SEQ ID NOs: 464, 465, and 466, respectively, with collectively a total of one or more amino acid positions in SEQ ID NOs: 464, 465, and 466 substituted with a histidine; (d) an HCVD including a CDR1, a CDR2, and a CDR3 of SEQ ID NOs: 544, 545, and 546, respectively, with collectively a total of one or more amino acid positions in SEQ ID NOs: 544, 545, and 546 optionally substituted with a histidine; and/or an LCVD including a CDR1, a CDR2, and a CDR3 of SEQ ID NOs: 547, 548, and 549, respectively, with collectively a total of one or more amino acid positions in SEQ ID NOs: 547, 548, and 549 optionally substituted with a histidine; (e) an HCVD including a CDR1, a CDR2, and a CDR3 of SEQ ID NOs: 630, 631, and 632, respectively, with collectively a total of one or more amino acid positions in SEQ ID NOs: 630, 631, and 632 optionally substituted with a histidine; and/or an LCVD including a CDR1, a CDR2, and a CDR3 of SEQ ID NOs: 633, 634, and 635, respectively, with collectively a total of one or more amino acid positions in SEQ ID NOs: 633, 634, and 635 substituted with a histidine; (f) an HCVD including a CDR1, a CDR2, and a CDR3 of SEQ ID NOs: 712, 713, and 714, respectively, with collectively a total of one or more amino acid positions in SEQ ID NOs: 712, 713, and 714 substituted with a histidine; and/or an LCVD including a CDR1, a CDR2, and a CDR3 of SEQ ID NOs: 715, 716, and 717, respectively, with collectively a total of one or more amino acid positions in SEQ ID NOs: 715, 716, and 717 substituted with a histidine; and (g) an HCVD including a CDR1, a CDR2, and a CDR3 of SEQ ID NOs: 805, 806, and 807, respectively, with collectively a total of one or more amino acid positions in SEQ ID NOs: 805, 806, and 807 optionally substituted with a histidine; and/or an LCVD including a CDR1, a CDR2, and a CDR3 of SEQ ID NOs: 808, 809, and 810, respectively, with collectively a total of one or more amino acid positions in SEQ ID NOs: 808, 809, and 810 substituted with a histidine.

In some embodiments, the first ABD includes an HCVD of one of: (a) an HCVD that is at least 90% identical to SEQ ID NO: 301, where the HCVD optionally includes a histidine at one or more positions in SEQ ID NO: 301 selected from: 29, 30, 32, 50-54, 58, 60, 96, and 101; (b) an HCVD that is at least 90% identical to SEQ ID NO: 375, where the HCVD optionally includes a histidine at one or more positions in SEQ ID NO: 375 selected from: 26, 27, 30, 32, 34, 51, 52, 55, 58, 59, 63, 97, 99, and 108; (c) an HCVD that is at least 90% identical to SEQ ID NO: 459, where the HCVD optionally includes a histidine at one or more positions in SEQ ID NO: 459 selected from: 32, 99, 101, 102, 106 and 111; (d) an HCVD that is at least 90% identical to SEQ ID NO: 542, where the HCVD optionally includes a histidine at one or more positions in SEQ ID NO: 542 selected from: 27, 29, 30, 31, 32, 53, 55, 58, 102, 104 and 105; (e) an HCVD that is at least 90% identical to SEQ ID NO: 628, where the HCVD optionally includes a histidine at one or more positions in SEQ ID NO: 628 selected from: 26, 28, 30, 50, 52, 53, 54, 55, 56, 57, 100, 101, 102 and 103; (f) an HCVD that is at least 90% identical to SEQ ID NO: 710, where the HCVD optionally includes a histidine at one or more positions in SEQ ID NO: 710 selected from: 29, 30, 31, 33, 35, 50, 53, 57, 58, 65, 96, 97, 98, 100, 101, 103, 105, 107, 108, 109, 110 and 113; and (g) an HCVD that is at least 90% identical to SEQ ID NO: 803, where the HCVD optionally includes a histidine at one or more positions in SEQ ID NO: 803 selected from: 33, 50, 53, 55-58, 64, 100, and 107.

In some embodiments, the first ABD includes an LCVD that is one of: (a) an LCVD that is at least 90% identical to SEQ ID NO: 302, where the LCVD optionally includes a histidine at one or more positions in SEQ ID NO: 302 selected from: 25, 30-33, 35, 55-57, 91, 94-96, and 98; (b) an LCVD that is at least 90% identical to SEQ ID NO: 376, where the LCVD optionally includes a histidine at one or more positions in SEQ ID NO: 376 selected from: 25, 29, 30, 31, 32, 33, 50, 51, 52, 53, 89, 91, 92, 93, 96, 97, 100 and 102; (c) an LCVD that is at least 90% identical to SEQ ID NO: 460, where the LCVD optionally includes a histidine at one or more positions in SEQ ID NO: 460 selected from: 30, 31, 33, 51, 92 and 95; (d) an LCVD that is at least 90% identical to SEQ ID NO: 543, where the LCVD optionally includes a histidine at one or more positions in SEQ ID NO: 543 selected from: 25, 29, 30, 31, 32, 89, 92 and 98; (e) an LCVD that is at least 90% identical to SEQ ID NO: 629, where the LCVD optionally includes a histidine at one or more positions in SEQ ID NO: 629 selected from: 25, 26, 28, 29, 33, 51, 55, 89, 91, 95, 96, 97, 98, 99 and 100; (f) an LCVD that is at least 90% identical to SEQ ID NO: 711, where the LCVD optionally includes a histidine at one or more positions in SEQ ID NO: 711 selected from: 25, 27, 29, 30, 35, 36, 56, 57, 91, 93, 94, 95, 98 and 99; and (g) an LCVD that is at least 90% identical to SEQ ID NO: 804, where the LCVD optionally includes a histidine at one or more positions in SEQ ID NO: 804 selected from: 31, 33-35, 52, 54, 98, and 99.

In some embodiments, the first ABD includes an HCVD of one of: (a) an HCVD that is at least 90% identical to SEQ ID NO: 301, where the HCVD optionally includes a histidine at two or more positions in SEQ ID NO: 301 selected from: 29, 30, 32, 50-54, 58, 60, 96, and 101; (b) an HCVD that is at least 90% identical to SEQ ID NO: 375, where the HCVD optionally includes a histidine at two or more positions in SEQ ID NO: 375 selected from: 26, 27, 30, 32, 34, 51, 52, 55, 58, 59, 63, 97, 99, and 108; (c) an HCVD that is at least 90% identical to SEQ ID NO: 459, where the HCVD optionally includes a histidine at two or more positions in SEQ ID NO: 459 selected from: 32, 99, 101, 102, 106 and 111; (d) an HCVD that is at least 90% identical to SEQ ID NO: 542, where the HCVD optionally includes a histidine at two or more positions in SEQ ID NO: 542 selected from: 27, 29, 30, 31, 32, 53, 55, 58, 102, 104 and 105; (e) an HCVD that is at least 90% identical to SEQ ID NO: 628, where the HCVD optionally includes a histidine at two or more positions in SEQ ID NO: 628 selected from: 26, 28, 30, 50, 52, 53, 54, 55, 56, 57, 100, 101, 102 and 103; (f) an HCVD that is at least 90% identical to SEQ ID NO: 710, where the HCVD optionally includes a histidine at two or more positions in SEQ ID NO: 710 selected from: 29, 30, 31, 33, 35, 50, 53, 57, 58, 65, 96, 97, 98, 100, 101, 103, 105, 107, 108, 109, 110 and 113; and (g) an HCVD that is at least 90% identical to SEQ ID NO: 803, where the HCVD optionally includes a histidine at two or more positions in SEQ ID NO: 803 selected from: 33, 50, 53, 55-58, 64, 100, and 107.

In some embodiments, the first ABD includes an LCVD that is one of: (a) an LCVD that is at least 90% identical to SEQ ID NO: 302, where the LCVD optionally includes a histidine at two or more positions in SEQ ID NO: 302 selected from: 25, 30-33, 35, 55-57, 91, 94-96, and 98; (b) an LCVD that is at least 90% identical to SEQ ID NO: 376, where the LCVD optionally includes a histidine at two or more positions in SEQ ID NO: 376 selected from: 25, 29, 30, 31, 32, 33, 50, 51, 52, 53, 89, 91, 92, 93, 96, 97, 100 and 102; (c) an LCVD that is at least 90% identical to SEQ ID NO: 460, where the LCVD optionally includes a histidine at two or more positions in SEQ ID NO: 460 selected from: 30, 31, 33, 51, 92 and 95; (d) an LCVD that is at least 90% identical to SEQ ID NO: 543, where the LCVD optionally includes a histidine at two or more positions in SEQ ID NO: 543 selected from: 25, 29, 30, 31, 32, 89, 92 and 98; (e) an LCVD that is at least 90% identical to SEQ ID NO: 629, where the LCVD optionally includes a histidine at two or more positions in SEQ ID NO: 629 selected from: 25, 26, 28, 29, 33, 51, 55, 89, 91, 95, 96, 97, 98, 99 and 100; (f) an LCVD that is at least 90% identical to SEQ ID NO: 711, where the LCVD optionally includes a histidine at two or more positions in SEQ ID NO: 711 selected from: 25, 27, 29, 30, 35, 36, 56, 57, 91, 93, 94, 95, 98 and 99; and (g) an LCVD that is at least 90% identical to SEQ ID NO: 804, where the LCVD optionally includes a histidine at two or more positions in SEQ ID NO: 804 selected from: 31, 33-35, 52, 54, 98, and 99.

In some embodiments, the first ABD includes one of: (a) an HCVD that is at least 90% identical to SEQ ID NO: 301, where the HCVD optionally includes a histidine at one or more positions in SEQ ID NO: 301 selected from: 29, 30, 32, 50-54, 58, 60, 96, and 101; and/or an LCVD that is at least 90% identical to SEQ ID NO: 302, where the LCVD optionally includes a histidine at one or more positions in SEQ ID NO: 302 selected from: 25, 30-33, 35, 55-57, 91, 94-96, and 98; (b) an HCVD that is at least 90% identical to SEQ ID NO: 375, where the HCVD optionally includes a histidine at one or more positions in SEQ ID NO: 375 selected from: 26, 27, 30, 32, 34, 51, 52, 55, 58, 59, 63, 97, 99, and 108; and/or an LCVD that is at least 90% identical to SEQ ID NO: 376, where the LCVD optionally includes a histidine at one or more positions in SEQ ID NO: 376 selected from: 25, 29, 30, 31, 32, 33, 50, 51, 52, 53, 89, 91, 92, 93, 96, 97, 100 and 102; (c) an HCVD that is at least 90% identical to SEQ ID NO: 459, where the HCVD optionally includes a histidine at one or more positions in SEQ ID NO: 459 selected from: 32, 99, 101, 102, 106 and 111; and/or an LCVD that is at least 90% identical to SEQ ID NO: 460, where the LCVD optionally includes a histidine at one or more positions in SEQ ID NO: 460 selected from 30, 31, 33, 51, 92 and 95; (d) an HCVD that is at least 90% identical to SEQ ID NO: 542, where the HCVD optionally includes a histidine at one or more positions in SEQ ID NO: 542 selected from: 27, 29, 30, 31, 32, 53, 55, 58, 102, 104 and 105; and/or an LCVD that is at least 90% identical to SEQ ID NO: 543, where the LCVD optionally includes a histidine at one or more positions in SEQ ID NO: 543 selected from 25, 29, 30, 31, 32, 89, 92 and 98; (e) an HCVD that is at least 90% identical to SEQ ID NO: 628, where the HCVD optionally includes a histidine at one or more positions in SEQ ID NO: 628 selected from: 26, 28, 30, 50, 52, 53, 54, 55, 56, 57, 100, 101, 102 and 103; and/or an LCVD that is at least 90% identical to SEQ ID NO: 629, where the LCVD optionally includes a histidine at one or more positions in SEQ ID NO: 629 selected from: 25, 26, 28, 29, 33, 51, 55, 89, 91, 95, 96, 97, 98, 99 and 100; (f) an HCVD that is at least 90% identical to SEQ ID NO: 710, where the HCVD optionally includes a histidine at one or more positions in SEQ ID NO: 710 selected from: 29, 30, 31, 33, 35, 50, 53, 57, 58, 65, 96, 97, 98, 100, 101, 103, 105, 107, 108, 109, 110 and 113; and/or an LCVD that is at least 90% identical to SEQ ID NO: 711, where the LCVD optionally includes a histidine at one or more positions in SEQ ID NO: 711 selected from 25, 27, 29, 30, 35, 36, 56, 57, 91, 93, 94, 95, 98 and 99; and (g) an HCVD that is at least 90% identical to SEQ ID NO: 803, where the HCVD optionally includes a histidine at one or more positions in SEQ ID NO: 803 selected from: 33, 50, 53, 55-58, 64, 100, and 107; and/or an LCVD that is at least 90% identical to SEQ ID NO: 804, where the LCVD optionally includes a histidine at one or more positions in SEQ ID NO: 804 selected from 31, 33-35, 52, 54, 98, and 99.

In some embodiments, the first ABD includes an HCVD of one of: (a) an HCVD of SEQ ID NO: 301, or one of 309-342; (b) an HCVD of SEQ ID NO: 375, or one of 383-423; (c) an HCVD of SEQ ID NO: 459, or one of 467-511; (d) an HCVD of SEQ ID NO: 542, or one of 550-592; (e) an HCVD of SEQ ID NO: 628, or one of 636-675; (f) an HCVD of SEQ ID NO: 710, or one of 718-767; and (g) an HCVD of SEQ ID NO: 803, or one of 811-854; and/or the first ABD includes an LCVD of one of: (a) an LCVD of SEQ ID NO: 302, or one of 343-374; (b) an LCVD of SEQ ID NO: 376, or one of 424-458; (c) an LCVD of SEQ ID NO: 460, or one of 512-541; (d) an LCVD of SEQ ID NO: 543, or one of 593-627; (e) an LCVD of SEQ ID NO: 629, or one of 676-709; (f) an LCVD of SEQ ID NO: 711, or one of 768-802; and (g) an LCVD of SEQ ID NO: 804, or one of 855-892.

In some embodiments, the first ABD includes one of:
(a) an HCVD of SEQ ID NO: 301, or one of 309-342, and/or an LCVD of SEQ ID NO: 302, or one of 343-374, where the first ABD does not include (i) an HCVD of SEQ ID NO: 301 and an LCVD of SEQ ID NO: 302; (ii) an HCVD of SEQ ID NO: 301 and an LCVD that is not one of SEQ ID NO: 344, 349-352, 354, 359-361, 363, 366-368, 370, and 373; or (iii) an HCVD that is not one of SEQ ID NO: 312, 313, 315, 319-323, 327, 329, 335, 340 and 341 and an LCVD of SEQ ID NO: 302;
(b) an HCVD of SEQ ID NO: 375, or one of 383-423; and/or an LCVD of SEQ ID NO: 376, or one of 424-458, where the first ABD does not include (i) an HCVD of SEQ ID NO: 375 and an LCVD of SEQ ID NO: 376; (ii) an HCVD of SEQ ID NO: 375 and an LCVD that is not one of SEQ ID NO: 425, 429, 430, 431, 432, 433, 435, 436, 437, 438, 442, 444, 445, 446, 449, 450, 453, and 455; or (iii) an HCVD that is not one of SEQ ID NO: 383, 384, 387, 389, 391, 394, 395, 398, 401, 402, 406, 410, 412, and 421 and an LCVD of SEQ ID NO: 376;
(c) an HCVD of SEQ ID NO: 459, or one of 467-511; and/or an LCVD of SEQ ID NO: 460, or one of 512-541, where the first ABD does not include (i) an HCVD of SEQ ID NO: 459 and an LCVD of SEQ ID NO: 460; (ii) an HCVD of SEQ ID NO: 459 and an LCVD that is not one of SEQ ID NO: 518, 519, 521, 524, 533, and 536; or (iii) an HCVD that is not one of SEQ ID NO: 473, 496, 498, 499, 503, and 508 and an LCVD of SEQ ID NO: 460;
(d) an HCVD of SEQ ID NO: 542, or one of 550-592; and/or an LCVD of SEQ ID NO: 543, or one of 593-627, where the first ABD does not include (i) an HCVD of SEQ ID NO: 542 and an LCVD of SEQ ID NO: 543; (ii) an HCVD of SEQ ID NO: 542 and an LCVD that is not one of SEQ ID NO: 594, 598, 599, 600, 601, 611, 614, and 620; or (iii) an HCVD that is not one of SEQ ID NO: 551, 553, 554, 555, 556, 563, 565, 568, 582, 584, and 585 and an LCVD of SEQ ID NO: 543;
(e) an HCVD of SEQ ID NO: 628, or one of 636-675; and/or an LCVD of SEQ ID NO: 629, or one of 676-709, where the first ABD does not include (i) an HCVD of SEQ ID NO: 628 and an LCVD of SEQ ID NO: 629; (ii) an HCVD of SEQ ID NO: 628 and an LCVD that is not one of SEQ ID NO: 677, 678, 680, 681, 685, 688, 692, 694, 696, 700, 701, 702, 703, 704, and 705; or (iii) an HCVD that is not one of SEQ ID NO: 636, 638, 640, 646, 648, 649, 650, 651, 652, 653, 666, 667, 668, and 669 and an LCVD of SEQ ID NO: 629;
(f) an HCVD of SEQ ID NO: 710, or one of 718-767; and/or an LCVD of SEQ ID NO: 711, or one of 768-802, where the first ABD does not include (i) an HCVD of SEQ ID NO: 710 and an LCVD of SEQ ID NO: 711; (ii) an HCVD of SEQ ID NO: 710 and an LCVD that is not one of SEQ ID NO: 769, 771, 773, 774, 779, 780, 785, 786, 788, 790, 791, 792, 795, and 796; or (iii) an HCVD that is not one of SEQ ID NO: 721, 722, 723, 725, 727, 728, 731, 735, 736, 743, 744, 745, 746, 748, 749, 751, 753, 755, 756, 757, 758, and 761 and an LCVD of SEQ ID NO: 711; and
(g) an HCVD of SEQ ID NO: 803, or one of 811-854; and/or an LCVD of SEQ ID NO: 804, or one of 855-892, where the first ABD does not include (i) an HCVD of SEQ ID NO: 803 and an LCVD of SEQ ID NO: 804; (ii) an HCVD of SEQ ID NO: 803 and an LCVD that is not one of SEQ ID NO: 862, 864-866, 868, 870, 881, and 882; or (iii) an HCVD that is not one of SEQ ID NO: 818, 821, 824, 826-829, 835, 841, 848, and 850 and an LCVD of SEQ ID NO: 804.

In some embodiments, the first antigen-binding domain includes an HCVD of one of: (a) an HCVD including a CDR1, a CDR2, and a CDR3 of SEQ ID NOs: 3, 4, and 5, respectively, with collectively a total of one or more amino acid positions in SEQ ID NOs: 3, 4, and 5 substituted with a histidine; (b) an HCVD including a CDR1, a CDR2, and a CDR3 of SEQ ID NOs: 128, 129, and 130, respectively, with collectively a total of one or more amino acid positions in SEQ ID NOs: 128, 129, and 130 substituted with a histidine; and (c) an HCVD including a CDR1, a CDR2, and a CDR3 of SEQ ID NOs: 231, 232, and 233, respectively, with collectively a total of one or more amino acid positions in SEQ ID NOs: 231, 232, and 233 substituted with a histidine. In some embodiments, the first CCK4-binding domain includes an LCVD of one of: (a) an LCVD including a CDR1, a CDR2, and a CDR3 of SEQ ID NOs: 6, 7, and 8, respectively, with collectively a total of one or more amino acid positions in SEQ ID NOs: 6, 7, and 8 substituted with a histidine; (b) an LCVD including a CDR1, a CDR2, and a CDR3 of SEQ ID NOs: 131, 132, and 133, respectively, with collectively a total of one or more amino acid positions in SEQ ID NOs: 131, 132, and 133 substituted with a histidine; and (c) an LCVD including a CDR1, a CDR2, and a CDR3 of SEQ ID NOs: 234, 235, and 236, respectively, with collectively a total of one or more amino acid positions in SEQ ID NOs: 234, 235, and 236 substituted with a histidine.

In some embodiments, the first CCK4-binding domain includes one of: (a) an HCVD including a CDR1, a CDR2, and a CDR3 of SEQ ID NOs: 3, 4, and 5, respectively, with collectively a total of one or more amino acid positions in SEQ ID NOs: 3, 4, and 5 substituted with a histidine; and/or an LCVD including a CDR1, a CDR2, and a CDR3 of SEQ ID NOs: 6, 7, and 8, respectively, with collectively a total of one or more amino acid positions in SEQ ID NOs: 6, 7, and 8 substituted with a histidine; (b) an HCVD including a CDR1, a CDR2, and a CDR3 of SEQ ID NOs: 128, 129, and 130, respectively, with collectively a total of one or more amino acid positions in SEQ ID NOs: 128, 129, and 130 substituted with a histidine; and/or an LCVD including a CDR1, a CDR2, and a CDR3 of SEQ ID NOs: 131, 132, and 133, respectively, with collectively a total of one or more amino acid positions in SEQ ID NOs: 131, 132, and 133 substituted with a histidine; and (c) an HCVD including a CDR1, a CDR2, and a CDR3 of SEQ ID NOs: 231, 232, and 233, respectively, with collectively a total of one or more amino acid positions in SEQ ID NOs: 231, 232, and 233 substituted with a histidine; and/or an LCVD including a CDR1, a CDR2, and a CDR3 of SEQ ID NOs: 234, 235, and 236, respectively, with collectively a total of one or more amino acid positions in SEQ ID NOs: 234, 235, and 236 substituted with a histidine.

In some embodiments, the first antigen-binding domain includes an HCVD of one of: (a) an HCVD that is at least 90% identical to SEQ ID NO: 1, where the HCVD includes a histidine at one or more positions in SEQ ID NO: 1 selected from the group consisting of: 24, 27, 28, 29, 30, 31, 32, 34, 53, 54, 55, 57, 58, 64, 98, 100, 102, 103, and 107; (b) an HCVD that is at least 90% identical to SEQ ID NO: 126, where the HCVD includes a histidine at one or more positions in SEQ ID NO: 126 selected from the group consisting of: 53, 54, 56, 57, 60, 102, 103, 104, 105, 106, 108, 109, 110, or 111; and (c) an HCVD that is at least 90% identical to SEQ ID NO: 229, where the HCVD includes a histidine at one or more positions in SEQ ID NO: 229 selected from the group consisting of: 27, 32, 33, 34, 35, 51, 54, 56, 58, or 100.

In some embodiments, the first antigen-binding domain includes an LCVD that is one of: (a) an LCVD that is at least 90% identical to SEQ ID NO: 2, where the LCVD includes a histidine at one or more positions in SEQ ID NO: 2 selected from the group consisting of: 25, 29, 31, 35, 60, 93, or 94; (b) an LCVD that is at least 90% identical to SEQ ID NO: 127, where the LCVD includes a histidine at one or more positions in SEQ ID NO: 127 selected from the group consisting of: 25, 26, 28, 29, 31, 32, 33, 50, 90, 92, and 94; and (c) an LCVD that is at least 90% identical to SEQ ID NO: 230, where the LCVD includes a histidine at one or more positions in SEQ ID NO: 230 selected from the group consisting of: 30, 31, 33, 51, 53, 57, 91, 92, 94, and 99.

In some embodiments, the first antigen-binding domain includes an HCVD of one of: (a) an HCVD that is at least 90% identical to SEQ ID NO: 1, where the HCVD includes a histidine at two or more positions in SEQ ID NO: 1 selected from the group consisting of: 27, 29, 31, 34, and 53; and (b) an HCVD that is at least 90% identical to SEQ ID NO: 126, where the HCVD includes a histidine at two or more positions in SEQ ID NO: 126 selected from the group consisting of: 53, 56, 57, 102, and 105.

In some embodiments, the first antigen-binding domain includes one of: (a) an HCVD that is at least 90% identical to SEQ ID NO: 1, where the HCVD includes a histidine at one or more positions in SEQ ID NO: 1 selected from the group consisting of: 24, 27, 28, 29, 30, 31, 32, 34, 53, 54, 55, 57, 58, 64, 98, 100, 102, 103, and 107; and/or an LCVD that is at least 90% identical to SEQ ID NO: 2, where the LCVD includes a histidine at one or more positions in SEQ ID NO: 2 selected from the group consisting of: 25, 29, 31, 35, 60, 93, or 94; (b) an HCVD that is at least 90% identical to SEQ ID NO: 126, where the HCVD includes a histidine at one or more positions in SEQ ID NO: 126 selected from the group consisting of: 53, 54, 56, 57, 60, 102, 103, 104, 105, 106, 108, 109, 110, or 111; and/or an LCVD that is at least 90% identical to SEQ ID NO: 127, where the LCVD includes a histidine at one or more positions in SEQ ID NO: 127 selected from the group consisting of: 25, 26, 28, 29, 31, 32, 33, 50, 90, 92, and 94; and (c) an HCVD that is at least 90% identical to SEQ ID NO: 229, where the HCVD includes a histidine at one or more positions in SEQ ID NO: 229 selected from the group consisting of: 27, 32, 33, 34, 35, 51, 54, 56, 58, or 100; and/or an LCVD that is at least 90% identical to SEQ ID NO: 230, where the LCVD includes a histidine at one or more positions in SEQ ID NO: 230 selected from the group consisting of: 30, 31, 33, 51, 53, 57, 91, 92, 94, and 99.

In some embodiments, the first antigen-binding domain includes an HCVD of one of: (a) an HCVD of SEQ ID NO: 1, 14, 17, 18, 19, 20, 21, 22, 24, 25, 29, 30, 31, 33, 34, 40, 44, 46, 48, 49, 53, 86, 87, 88, 89, 90, 91, 92, 93, 94, 95, 96, 98, 99, 100, 101, 102, 103, 104, or 105; (b) an HCVD of SEQ ID NO: 126, 147, 148, 150, 151, 154, 166, 167, 168, 169, 170, 172, 173, 174, 175, 208, 209, 210, 211, 212, 213, 214, 215, 216, 217, 218, 219, 220, 221, 222, 224, 225, 226, or 227; and (c) an HCVD of SEQ ID NO: 229, 238, 243, 244, 245, 246, 248, 251, 253, 255, or 267.

In some embodiments, the first antigen-binding domain includes an LCVD of one of: (a) an LCVD of SEQ ID NO: 2, 56, 60, 62, 66, 69, 76, 77, 78, 110, 113, 114, 119, 122, 123, 124, or 125; (b) an LCVD of SEQ ID NO: 127, 181, 182, 184, 185, 187, 188, 189, 191, 199, 201, 203, or 228; and (c) an LCVD of SEQ ID NO: 230, 275, 276, 278, 281, 283, 287, 289, 290, 292, or 297.

In some embodiments, the first antigen binding domain includes one of: (a) an HCVD of SEQ ID NO: 14, 17, 18, 19, 20, 21, 22, 24, 25, 29, 30, 31, 33, 34, 40, 44, 46, 48, 49, 53, 86, 87, 88, 89, 90, 91, 92, 93, 94, 95, 96, 98, 99, 100, 101, 102, 103, 104, or 105, and/or an LCVD of SEQ ID NO: 2, 56, 60, 62, 66, 69, 76, 77, 78, 110, 113, 114, 119, 122, 123, 124, or 125, where the first antigen binding domain does not include (i) an HCVD of SEQ ID NO: 1 and an LCVD of SEQ ID NO: 2; (ii) an HCVD of SEQ ID NO: 1 and LCVD that is not one of SEQ ID NOs: SEQ ID NOs: 56, 60, 62, 66, 69, 76, 77, 78, 110, 113, 114, 119, and 122-125; or (iii) an LCVD of SEQ ID NO: 2 and an HCVD that is not one of SEQ ID NOs: 14, 17-22, 24, 25, 29-31, 33, 34, 40, 44, 46, 48, 49, 53, 86-96, and 98-105; (b) an HCVD of SEQ ID NO: 147, 148, 150, 151, 154, 166, 167, 168, 169, 170, 172, 173, 174, 175, 208, 209, 210, 211, 212, 213, 214, 215, 216, 217, 218, 219, 220, 221, 222, 224, 225, 226, or 227; and/or an LCVD of SEQ ID NO: 127, 181, 182, 184, 185, 187, 188, 189, 191, 199, 201, 203, or 228, where the first antigen-binding domain does not include (i) an HCVD of SEQ ID NO: 126 and an LCVD of SEQ ID NO: 127; (ii) an HCVD of SEQ ID NO: 126 and an LCVD that is not one of SEQ ID NOs: 181, 182, 184, 185, 187-189, 191, 199, 201, 203, and 228; (iii) an LCVD of SEQ ID NO: 127 and an HCVD that is not one of SEQ ID NOs: 147, 148, 150, 151, 154, 166-170, 172-175, 208-222, and 224-227; and (c) an HCVD of SEQ ID NO: 238, 243, 244, 245, 246, 248, 251, 253, 255, or 267; and/or an LCVD of SEQ ID NO: 230, 275, 276, 278, 281, 283, 287, 289, 290, 292, or 297, where the first antigen-binding domain does not include (i) an HCVD of SEQ ID NO: 229 and an LCVD of SEQ ID NO: 230; (ii) an HCVD of SEQ ID NO: 229 and an LCVD that is not one of SEQ ID NOs: 275, 276, 278, 281, 283, 287, 289, 290, 292, and 297; and (iii) an LCVD of SEQ ID NO: 230 and an HCVD that is not one of SEQ ID NOs: 238, 243-246, 248, 251, 253, 255, and 267.

In some embodiments, the ABPC is degraded in the target mammalian cell following internalization of the ABPC by the target mammalian cell. In some embodiments, the ABPC includes a conjugated toxin, radioisotope, drug, or small molecule.

In some embodiments, a composition including the ABPC provides for an increase in toxin liberation in the target mammalian cell as compared to a composition including the same amount of a control ABPC. In some embodiments, a composition including the ABPC provides for at least a 20% r 50% increase in toxin liberation in the target mammalian cell as compared to a composition including the same amount of a control ABPC. In some embodiments, a composition including the ABPC provides for at least a 2- or 5-fold increase in toxin liberation in the target mammalian cell as compared to a composition including the same amount of a control ABPC.

In some embodiments, a composition including the ABPC provides for an increase in target mammalian cell killing as compared to a composition including the same amount of a control ABPC. In some embodiments, a composition including the ABPC provides for at least a 20% r 50% increase in target mammalian cell killing as compared to a composition including the same amount of a control ABPC. In some embodiments, a composition including the ABPC provides for at least a 2- or 5-fold increase in target mammalian cell killing as compared to a composition including the same amount of a control ABPC.

In some embodiments, a composition including the ABPC provides for an increase in endolysosomal delivery in the target mammalian cell as compared to a composition including the same amount of a control ABPC. In some embodiments, the ABPC provides for at least a 20% increase in endolysosomal delivery in the target mammalian cell as compared to a composition including the same amount of a control ABPC. In some embodiments, a composition including the ABPC provides for at least a 50% increase in endolysosomal delivery in the target mammalian cell as compared to a composition including the same amount of a control ABPC. In some embodiments, a composition including the ABPC provides for at least a 2-fold increase in endolysosomal delivery in the target mammalian cell as compared to a composition including the same amount of a control ABPC. In some embodiments, a composition including the ABPC provides for at least a 5-fold increase in endolysosomal delivery in the target mammalian cell as compared to a composition including the same amount of a control ABPC.

In some embodiments, a composition including the ABPC results in a less of a reduction in the level of PTK7 presented on the surface of the target mammalian cell as compared to a composition including the same amount of a control ABPC. In some embodiments, a composition including the ABPC does not result in a detectable reduction in the level of the PTK7 presented on the surface of the target mammalian cell.

Also provided herein are antigen-binding protein constructs (ABPCs) including: a first ABD that is capable of specifically binding PTK7 or an epitope of PTK7 presented on the surface of a target mammalian cell; and a conjugated toxin, radioisotope, drug, or small molecule, where: (a) the dissociation rate of the first ABD at a pH of about 4.0 to about 6.5 is faster than the dissociation rate at a pH of about 7.0 to about 8.0; or the dissociation constant (K_{D}) of the first ABD at a pH of about 4.0 to about 6.5 is greater than the K_{D} at a pH of about 7.0 to about 8.0; and (b) the composition provides for one or more of: an increase in toxin liberation in the target mammalian cell as compared to a composition including the same amount of a control ABPC; an increase in target mammalian cell killing as compared to a composition including the same amount of a control ABPC; and an increase in endolysosomal delivery in the target mammalian cell as compared to a composition including the same amount of a control ABPC.

In some embodiments, the first ABD includes one of: (a) an HCVD of MYT9345; (b) an HCVD of MYT9359; (c) an HCVD of MYT9361; (d) an HCVD of MYT9412; (e) an HCVD of MYT9460; (f) an HCVD of MYT9792; (g) an HCVD of MYT9797, (h) an HCVD of cofetuzumab; (i) an HCVD of 7C8; and (j) an HCVD of 12C6, each or any with one or more amino acids optionally substituted with a histidine. In some embodiments, the first ABD includes one of: (a) an LCVD of MYT9345; (b) an LCVD of MYT9359; (c) an LCVD of MYT9361; (d) an LCVD of MYT9412; (e) an LCVD of MYT9460; (f) an LCVD of MYT9792; (g) an LCVD of MYT9797, (h) an LCVD of cofetuzumab; (i) an LCVD of 7C8; and (j) an LCVD of 12C6, each or any optionally with one or more amino acids substituted with a histidine.

In some embodiments, the first ABD includes one of: (a) an HCVD of MYT9345 and an LCVD of MYT9345; (b) an HCVD of MYT9359 and an LCVD of MYT9359; (c) an HCVD of MYT9361 and an LCVD of MYT9361; (d) an HCVD of MYT9412 and an LCVD of MYT9412; (e) an HCVD of MYT9460 and an LCVD of MYT9460; (f) an HCVD of MYT9792 and an LCVD of MYT9792; (g) an HCVD of MYT9797 and an LCVD of MYT9797; (h) an HCVD of cofetuzumab and an LCVD of cofetuzumab; (i) an HCVD of 7C8 and an LCVD of 7C8; and (j) an HCVD of 12C6 and an LCVD of 12C6, each or any of the domains optionally with one or more amino acids substituted with a histidine.

In some embodiments, the HCVD includes one of: (a) an HCVD of MYT9345 including SEQ ID NO: 301; (b) an HCVD of MYT9359 including SEQ ID NO: 375; (c) an HCVD of MYT9361 including SEQ ID NO: 459; (d) an HCVD of MYT9412 including SEQ ID NO: 542; (e) an HCVD of MYT9460 including SEQ ID NO: 628; (f) an HCVD of MYT9792 including SEQ ID NO: 710; and (g) an HCVD of MYT9797 including SEQ ID NO: 803. In some embodiments, the LCVD includes one of: (a) an LCVD of MYT9345 including SEQ ID NO: 302; (b) an LCVD of MYT9359 including SEQ ID NO: 376; (c) an LCVD of MYT9361 including SEQ ID NO: 460; (d) an LCVD of MYT9412 including SEQ ID NO: 543; (e) an LCVD of MYT9460 including SEQ ID NO: 629; (f) an LCVD of MYT9792 including SEQ ID NO: 711; and (g) an LCVD of MYT9797 including SEQ ID NO: 804.

In some embodiments, the first ABD includes an HCVD of one of: (a) an HCVD including a CDR1, a CDR2, and a CDR3 of SEQ ID NOs: 303, 304, and 305, respectively, with collectively a total of one or more amino acid positions in SEQ ID NOs: 303, 304, and 305 substituted with a histidine; (b) an HCVD including a CDR1, a CDR2, and a CDR3 of SEQ ID NOs: 377, 378, and 379, respectively, with collectively a total of one or more amino acid positions in SEQ ID NOs: 377, 378, and 379 optionally substituted with a histidine; (c) an HCVD including a CDR1, a CDR2, and a CDR3 of SEQ ID NOs: 461, 462, and 463, respectively, with collectively a total of one or more amino acid positions in SEQ ID NOs: 461, 462, and 463 substituted with a histidine; (d) an HCVD including a CDR1, a CDR2, and a CDR3 of SEQ ID NOs: 544, 545, and 546, respectively, with collectively a total of one or more amino acid positions in SEQ ID NOs: 544, 545, and 546 optionally substituted with a histidine; (e) an HCVD including a CDR1, a CDR2, and a CDR3 of SEQ ID NOs: 630, 631, and 632, respectively, with collectively a total of one or more amino acid positions in SEQ ID NOs: 630, 631, and 632 optionally substituted with a histidine; (f) an HCVD including a CDR1, a CDR2, and a CDR3 of SEQ ID NOs: 712, 713, and 714, respectively, with collectively a total of one or more amino acid positions in SEQ ID NOs: 712, 713, and 714 substituted with a histidine; and (g) an HCVD including a CDR1, a CDR2, and a CDR3 of SEQ ID NOs: 805, 806, and 807, respectively, with collectively a total of one or more amino acid positions in SEQ ID NOs: 805, 806, and 807 substituted with a histidine.

In some embodiments, the first ABD includes an LCVD of one of: (a) an LCVD including a CDR1, a CDR2, and a CDR3 of SEQ ID NOs: 306, 307, and 308, respectively, with collectively a total of one or more amino acid positions in SEQ ID NOs: 306, 307, and 308 optionally substituted with a histidine; (b) an LCVD including a CDR1, a CDR2, and a CDR3 of SEQ ID NOs: 380, 381, and 382, respectively, with collectively a total of one or more amino acid positions in SEQ ID NOs: 380, 381, and 382 optionally substituted with a histidine; (c) an LCVD including a CDR1, a CDR2, and a CDR3 of SEQ ID NOs: 464, 465, and 466, respectively, with collectively a total of one or more amino acid positions in SEQ ID NOs: 464, 465, and 466 substituted with a histidine; (d) an LCVD including a CDR1, a CDR2, and a CDR3 of SEQ ID NOs: 547, 548, and 549, respectively, with collectively a total of one or more amino acid positions in SEQ ID NOs: 547, 548, and 549 optionally substituted with a histidine; (e) an LCVD including a CDR1, a CDR2, and a CDR3 of SEQ ID NOs: 633, 634, and 635, respectively, with collectively a total of one or more amino acid positions in SEQ ID NOs: 633, 634, and 635 substituted with a histidine; (f) an LCVD including a CDR1, a CDR2, and a CDR3 of SEQ ID NOs: 715, 716, and 717, respectively, with collectively a total of one or more amino acid positions in SEQ ID NOs: 715, 716, and 717 substituted with a histidine; and (g) an LCVD including a CDR1, a CDR2, and a CDR3 of SEQ ID NOs: 808, 809, and 810, respectively, with collectively a total of one or more amino acid positions in SEQ ID NOs: 808, 809, and 810 substituted with a histidine.

In some embodiments, the first ABD includes one of: (a) an HCVD including a CDR1, a CDR2, and a CDR3 of SEQ ID NOs: 303, 304, and 305, respectively, with collectively a total of one or more amino acid positions in SEQ ID NOs: 303, 304, and 305 substituted with a histidine; and/or an LCVD including a CDR1, a CDR2, and a CDR3 of SEQ ID NOs: 306, 307, and 308, respectively, with collectively a total of one or more amino acid positions in SEQ ID NOs: 306, 307, and 308 optionally substituted with a histidine; (b) an HCVD including a CDR1, a CDR2, and a CDR3 of SEQ ID NOs: 377, 378, and 379, respectively, with collectively a total of one or more amino acid positions in SEQ ID NOs: 377, 378, and 379 optionally substituted with a histidine; and/or an LCVD including a CDR1, a CDR2, and a CDR3 of SEQ ID NOs: 380, 381, and 382, respectively, with collectively a total of one or more amino acid positions in SEQ ID NOs: 380, 381, and 382 optionally substituted with a histidine; (c) an HCVD including a CDR1, a CDR2, and a CDR3 of SEQ ID NOs: 461, 462, and 463, respectively, with collectively a total of one or more amino acid positions in SEQ ID NOs: 461, 462, and 463 substituted with a histidine; and/or an LCVD including a CDR1, a CDR2, and a CDR3 of SEQ ID NOs: 464, 465, and 466, respectively, with collectively a total of one or more amino acid positions in SEQ ID NOs: 464, 465, and 466 substituted with a histidine; (d) an HCVD including a CDR1, a CDR2, and a CDR3 of SEQ ID NOs: 544, 545, and 546, respectively, with collectively a total of one or more amino acid positions in SEQ ID NOs: 544, 545, and 546 optionally substituted with a histidine; and/or an LCVD including a CDR1, a CDR2, and a CDR3 of SEQ ID NOs: 547, 548, and 549, respectively, with collectively a total of one or more amino acid positions in SEQ ID NOs: 547, 548, and 549 optionally substituted with a histidine; (e) an HCVD including a CDR1, a CDR2, and a CDR3 of SEQ ID NOs: 630, 631, and 632, respectively, with collectively a total of one or more amino acid positions in SEQ ID NOs: 630, 631, and 632 optionally substituted with a histidine; and/or an LCVD including a CDR1, a CDR2, and a CDR3 of SEQ ID NOs: 633, 634, and 635, respectively, with collectively a total of one or more amino acid positions in SEQ ID NOs: 633, 634, and 635 substituted with a histidine; (f) an HCVD including a CDR1, a CDR2, and a CDR3 of SEQ ID NOs: 712, 713, and 714, respectively, with collectively a total of one or more amino acid positions in SEQ ID NOs: 712, 713, and 714 substituted with a histidine; and/or an LCVD including a CDR1, a CDR2, and a CDR3 of SEQ ID NOs: 715, 716, and 717, respectively, with collectively a total of one or more amino acid positions in SEQ ID NOs: 715, 716, and 717 substituted with a histidine; and (g) an HCVD including a CDR1, a CDR2, and a CDR3 of SEQ ID NOs: 805, 806, and 807, respectively, with collectively a total of one or more amino acid positions in SEQ ID NOs: 805, 806, and 807 optionally substituted with a histidine; and/or an LCVD including a CDR1, a CDR2, and a CDR3 of SEQ ID NOs: 808, 809, and 810, respectively, with collectively a total of one or more amino acid positions in SEQ TD NOs: 808, 809, and 810 substituted with a histidine.

In some embodiments, the first ABD includes an HCVD of one of: (a) an HCVD that is at least 90% identical to SEQ ID NO: 301, where the HCVD optionally includes a histidine at one or more positions in SEQ ID NO: 301 selected from: 29, 30, 32, 50-54, 58, 60, 96, and 101; (b) an HCVD that is at least 90% identical to SEQ ID NO: 375, where the HCVD optionally includes a histidine at one or more positions in SEQ ID NO: 375 selected from: 26, 27, 30, 32, 34, 51, 52, 55, 58, 59, 63, 97, 99, and 108; (c) an HCVD that is at least 90% identical to SEQ ID NO: 459, where the HCVD optionally includes a histidine at one or more positions in SEQ ID NO: 459 selected from: 32, 99, 101, 102, 106 and 111; (d) an HCVD that is at least 90% identical to SEQ ID NO: 542, where the HCVD optionally includes a histidine at one or more positions in SEQ ID NO: 542 selected from: 27, 29, 30, 31, 32, 53, 55, 58, 102, 104 and 105; (e) an HCVD that is at least 90% identical to SEQ ID NO: 628, where the HCVD optionally includes a histidine at one or more positions in SEQ ID NO: 628 selected from: 26, 28, 30, 50, 52, 53, 54, 55, 56, 57, 100, 101, 102 and 103; (f) an HCVD that is at least 90% identical to SEQ ID NO: 710, where the HCVD optionally includes a histidine at one or more positions in SEQ ID NO: 710 selected from: 29, 30, 31, 33, 35, 50, 53, 57, 58, 65, 96, 97, 98, 100, 101, 103, 105, 107, 108, 109, 110 and 113; and (g) an HCVD that is at least 90% identical to SEQ ID NO: 803, where the HCVD optionally includes a histidine at one or more positions in SEQ ID NO: 803 selected from: 33, 50, 53, 55-58, 64, 100, and 107.

In some embodiments, the first ABD includes an LCVD of one of: (a) an LCVD that is at least 90% identical to SEQ ID NO: 302, where the LCVD optionally includes a histidine at one or more positions in SEQ ID NO: 302 selected from: 25, 30-33, 35, 55-57, 91, 94-96, and 98; (b) an LCVD that is at least 90% identical to SEQ ID NO: 376, where the LCVD optionally includes a histidine at one or more positions in SEQ ID NO: 376 selected from: 25, 29, 30, 31, 32, 33, 50, 51, 52, 53, 89, 91, 92, 93, 96, 97, 100 and 102; (c) an LCVD that is at least 90% identical to SEQ ID NO: 460, where the LCVD optionally includes a histidine at one or more positions in SEQ ID NO: 460 selected from: 30, 31, 33, 51, 92 and 95; (d) an LCVD that is at least 90% identical to SEQ ID NO: 543, where the LCVD optionally includes a histidine at one or more positions in SEQ ID NO: 543 selected from: 25, 29, 30, 31, 32, 89, 92 and 98; (e) an LCVD that is at least 90% identical to SEQ ID NO: 629, where the LCVD optionally includes a histidine at one or more positions in SEQ ID NO: 629 selected from: 25, 26, 28, 29, 33, 51, 55, 89, 91, 95, 96, 97, 98, 99 and 100; (f) an LCVD that is at least 90% identical to SEQ ID NO: 711, where the LCVD optionally includes a histidine at one or more positions in SEQ ID NO: 711 selected from: 25, 27, 29, 30, 35, 36, 56, 57, 91, 93, 94, 95, 98 and 99; and (g) an LCVD that is at least 90% identical to SEQ ID NO: 804, where the LCVD optionally includes a histidine at one or more positions in SEQ ID NO: 804 selected from: 31, 33-35, 52, 54, 98, and 99.

In some embodiments, the first ABD includes an HCVD of one of: (a) an HCVD that is at least 90% identical to SEQ ID NO: 301, where the HCVD optionally includes a histidine at two or more positions in SEQ ID NO: 301 selected from: 29, 30, 32, 50-54, 58, 60, 96, and 101; (b) an HCVD that is at least 90% identical to SEQ ID NO: 375, where the HCVD optionally includes a histidine at two or more positions in SEQ ID NO: 375 selected from: 26, 27, 30, 32, 34, 51, 52, 55, 58, 59, 63, 97, 99, and 108; (c) an HCVD that is at least 90% identical to SEQ ID NO: 459, where the HCVD optionally includes a histidine at two or more positions in SEQ ID NO: 459 selected from: 32, 99, 101, 102, 106 and 111; (d) an HCVD that is at least 90% identical to SEQ ID NO: 542, where the HCVD optionally includes a histidine at two or more positions in SEQ ID NO: 542 selected from: 27, 29, 30, 31, 32, 53, 55, 58, 102, 104 and 105; (e) an HCVD that is at least 90% identical to SEQ ID NO: 628, where the HCVD optionally includes a histidine at two or more positions in SEQ ID NO: 628 selected from: 26, 28, 30, 50, 52, 53, 54, 55, 56, 57, 100, 101, 102 and 103; (f) an HCVD that is at least 90% identical to SEQ ID NO: 710, where the HCVD optionally includes a histidine at two or more positions in SEQ ID NO: 710 selected from: 29, 30, 31, 33, 35, 50, 53, 57, 58, 65, 96, 97, 98, 100, 101, 103, 105, 107, 108, 109, 110 and 113; and (g) an HCVD that is at least 90% identical to SEQ ID NO: 803, where the HCVD optionally includes a histidine at two or more positions in SEQ ID NO: 803 selected from: 33, 50, 53, 55-58, 64, 100, and 107.

In some embodiments, the first ABD includes an LCVD of one of: (a) an LCVD that is at least 90% identical to SEQ ID NO: 302, where the LCVD optionally includes a histidine at two or more positions in SEQ ID NO: 302 selected from: 25, 30-33, 35, 55-57, 91, 94-96, and 98; (b) an LCVD that is at least 90% identical to SEQ ID NO: 376, where the LCVD optionally includes a histidine at two or more positions in SEQ ID NO: 376 selected from: 25, 29, 30, 31, 32, 33, 50, 51, 52, 53, 89, 91, 92, 93, 96, 97, 100 and 102; (c) an LCVD that is at least 90% identical to SEQ ID NO: 460, where the LCVD optionally includes a histidine at two or more positions in SEQ ID NO: 460 selected from: 30, 31, 33, 51, 92 and 95; (d) an LCVD that is at least 90% identical to SEQ ID NO: 543, where the LCVD optionally includes a histidine at two or more positions in SEQ ID NO: 543 selected from: 25, 29, 30, 31, 32, 89, 92 and 98; (e) an LCVD that is at least 90% identical to SEQ ID NO: 629, where the LCVD optionally includes a histidine at two or more positions in SEQ ID NO: 629 selected from: 25, 26, 28, 29, 33, 51, 55, 89, 91, 95, 96, 97, 98, 99 and 100; (f) an LCVD that is at least 90% identical to SEQ ID NO: 711, where the LCVD optionally includes a histidine at two or more positions in SEQ ID NO: 711 selected from: 25, 27, 29, 30, 35, 36, 56, 57, 91, 93, 94, 95, 98 and 99; and (g) an LCVD that is at least 90% identical to SEQ ID NO: 804, where the LCVD optionally includes a histidine at two or more positions in SEQ ID NO: 804 selected from: 31, 33-35, 52, 54, 98, and 99.

In some embodiments, the first ABD includes one of: (a) an HCVD that is at least 90% identical to SEQ ID NO: 301, where the HCVD optionally includes a histidine at one or more positions in SEQ ID NO: 301 selected from: 29, 30, 32, 50-54, 58, 60, 96, and 101; and/or an LCVD that is at least 90% identical to SEQ ID NO: 302, where the LCVD optionally includes a histidine at one or more positions in SEQ ID NO: 302 selected from: 25, 30-33, 35, 55-57, 91, 94-96, and 98; (b) an HCVD that is at least 90% identical to SEQ ID NO: 375, where the HCVD optionally includes a histidine at one or more positions in SEQ ID NO: 375 selected from: 26, 27, 30, 32, 34, 51, 52, 55, 58, 59, 63, 97, 99, and 108; and/or an LCVD that is at least 90% identical to SEQ ID NO: 376, where the LCVD optionally includes a histidine at one or more positions in SEQ ID NO: 376 selected from: 25, 29, 30, 31, 32, 33, 50, 51, 52, 53, 89, 91, 92, 93, 96, 97, 100 and 102; (c) an HCVD that is at least 90% identical to SEQ ID NO: 459, where the HCVD optionally includes a histidine at one or more positions in SEQ ID NO: 459 selected from: 32, 99, 101, 102, 106 and 111; and/or an LCVD that is at least 90% identical to SEQ ID NO: 460, where the LCVD optionally includes a histidine at one or more positions in SEQ ID NO: 460 selected from: 30, 31, 33, 51, 92 and 95; (d) an HCVD that is at least 90% identical to SEQ ID NO: 542, where the HCVD optionally includes a histidine at one or more positions in SEQ ID NO: 542 selected from: 27, 29, 30, 31, 32, 53, 55, 58, 102, 104 and 105; and/or an LCVD that is at least 90% identical to SEQ ID NO: 543, where the LCVD optionally includes a histidine at one or more positions in SEQ ID NO: 543 selected from: 25, 29, 30, 31, 32, 89, 92 and 98; (e) an HCVD that is at least 90% identical to SEQ ID NO: 628, where the HCVD optionally includes a histidine at one or more positions in SEQ ID NO: 628 selected from: 26, 28, 30, 50, 52, 53, 54, 55, 56, 57, 100, 101, 102 and 103; and/or an LCVD that is at least 90% identical to SEQ ID NO: 629, where the LCVD optionally includes a histidine at one or more positions in SEQ ID NO: 629 selected from: 25, 26, 28, 29, 33, 51, 55, 89, 91, 95, 96, 97, 98, 99 and 100; (f) an HCVD that is at least 90% identical to SEQ ID NO: 710, where the HCVD optionally includes a histidine at one or more positions in SEQ ID NO: 710 selected from: 29, 30, 31, 33, 35, 50, 53, 57, 58, 65, 96, 97, 98, 100, 101, 103, 105, 107, 108, 109, 110 and 113; and/or an LCVD that is at least 90% identical to SEQ ID NO: 711, where the LCVD optionally includes a histidine at one or more positions in SEQ ID NO: 711 selected from: 25, 27, 29, 30, 35, 36, 56, 57, 91, 93, 94, 95, 98 and 99; and (g) an HCVD that is at least 90% identical to SEQ ID NO: 803, where the HCVD optionally includes a histidine at one or more positions in SEQ ID NO: 803 selected from: 33, 50, 53, 55-58, 64, 100, and 107; and/or an LCVD that is at least 90% identical to SEQ ID NO: 804, where the LCVD optionally includes a histidine at one or more positions in SEQ ID NO: 804 selected from: 31, 33-35, 52, 54, 98, and 99.

In some embodiments, the first ABD includes one of: (a) an HCVD that is at least 90% identical to SEQ ID NO: 301, where the HCVD optionally includes a histidine at two or more positions in SEQ ID NO: 301 selected from: 29, 30, 32, 50-54, 58, 60, 96, and 101; and/or an LCVD that is at least 90% identical to SEQ ID NO: 302, where the LCVD optionally includes a histidine at two or more positions in SEQ ID NO: 302 selected from: 25, 30-33, 35, 55-57, 91, 94-96, and 98; (b) an HCVD that is at least 90% identical to SEQ ID NO: 375, where the HCVD optionally includes a histidine at two or more positions in SEQ ID NO: 375 selected from: 26, 27, 30, 32, 34, 51, 52, 55, 58, 59, 63, 97, 99, and 108; and/or an LCVD that is at least 90% identical to SEQ ID NO: 376, where the LCVD optionally includes a histidine at two or more positions in SEQ ID NO: 376 selected from: 25, 29, 30, 31, 32, 33, 50, 51, 52, 53, 89, 91, 92, 93, 96, 97, 100 and 102; (c) an HCVD that is at least 90% identical to SEQ ID NO: 459, where the HCVD optionally includes a histidine at two or more positions in SEQ ID NO: 459 selected from: 32, 99, 101, 102, 106 and 111; and/or an LCVD that is at least 90% identical to SEQ ID NO: 460, where the LCVD optionally includes a histidine at two or more positions in SEQ ID NO: 460 selected from: 30, 31, 33, 51, 92 and 95; (d) an HCVD that is at least 90% identical to SEQ ID NO: 542, where the HCVD optionally includes a histidine at two or more positions in SEQ ID NO: 542 selected from: 27, 29, 30, 31, 32, 53, 55, 58, 102, 104 and 105; and/or an LCVD that is at least 90% identical to SEQ ID NO: 543, where the LCVD optionally includes a histidine at two or more positions in SEQ ID NO: 543 selected from: 25, 29, 30, 31, 32, 89, 92 and 98; (e) an HCVD that is at least 90% identical to SEQ ID NO: 628, where the HCVD optionally includes a histidine at two or more positions in SEQ ID NO: 628 selected from: 26, 28, 30, 50, 52, 53, 54, 55, 56, 57, 100, 101, 102 and 103; and/or an LCVD that is at least 90% identical to SEQ ID NO: 629, where the LCVD optionally includes a histidine at two or more positions in SEQ ID NO: 629 selected from: 25, 26, 28, 29, 33, 51, 55, 89, 91, 95, 96, 97, 98, 99 and 100; (f) an HCVD that is at least 90% identical to SEQ ID NO: 710, where the HCVD optionally includes a histidine at two or more positions in SEQ ID NO: 710 selected from: 29, 30, 31, 33, 35, 50, 53, 57, 58, 65, 96, 97, 98, 100, 101, 103, 105, 107, 108, 109, 110 and 113; and/or an LCVD that is at least 90% identical to SEQ ID NO: 711, where the LCVD optionally includes a histidine at two or more positions in SEQ ID NO: 711 selected from: 25, 27, 29, 30, 35, 36, 56, 57, 91, 93, 94, 95, 98 and 99; and (g) an HCVD that is at least 90% identical to SEQ ID NO: 803, where the HCVD optionally includes a histidine at two or more positions in SEQ ID NO: 803 selected from: 33, 50, 53, 55-58, 64, 100, and 107; and/or an LCVD that is at least 90% identical to SEQ ID NO: 804, where the LCVD optionally includes a histidine at two or more positions in SEQ ID NO: 804 selected from: 31, 33-35, 52, 54, 98, and 99.

In some embodiments, the first ABD includes one of: (a) an HCVD of SEQ ID NO: 301, or one of 309-342; (b) an HCVD of SEQ ID NO: 375, or one of 383-423; (c) an HCVD of SEQ ID NO: 459, or one of 467-511, (d) an HCVD of SEQ ID NO: 542, or one of 550-592; (e) an HCVD of SEQ ID NO: 628, or one of 636-675; (f) an HCVD of SEQ ID NO: 710, or one of 718-767; and (g) an HCVD of SEQ ID NO: 803, or one of 811-854.

In some embodiments, the first ABD includes one of: (a) an LCVD of SEQ ID NO: 302, or one of 343-374; (b) an LCVD of SEQ ID NO: 376, or one of 424-458; (c) an LCVD of SEQ ID NO: 460, or one of 512-541; (d) an LCVD of SEQ ID NO: 543, or one of 593-627; (e) an LCVD of SEQ ID NO: 629, or one of 676-709; (f) an LCVD of SEQ ID NO: 711, or one of 768-802; and (g) an LCVD of SEQ ID NO: 804, or one of 855-892.

In some embodiments, the first ABD includes one of:
(a) an HCVD of SEQ ID NO: 301, or one of 309-342, and/or an LCVD of SEQ ID NO: 302, or one of 343-374, where the first ABD does not include (i) an HCVD of SEQ ID NO: 301 and an LCVD of SEQ ID NO: 302; (ii) an HCVD of SEQ ID NO: 301 and an LCVD that is not one of SEQ ID NO: 344, 349-352, 354, 359-361, 363, 366-368, 370, and 373; or (iii) an HCVD that is not one of SEQ ID NO: 312, 313, 315, 319-323, 327, 329, 335, 340 and 341 and an LCVD of SEQ ID NO: 302;
(b) an HCVD of SEQ ID NO: 375, or one of 383-423; and/or an LCVD of SEQ ID NO: 376, or one of 424-458, where the first ABD does not include (i) an HCVD of SEQ ID NO: 375 and an LCVD of SEQ ID NO: 376; (ii) an HCVD of SEQ ID NO: 375 and an LCVD that is not one of SEQ ID NO: 425, 429, 430, 431, 432, 433, 435, 436, 437, 438, 442, 444, 445, 446, 449, 450, 453, and 455; or (iii) an HCVD that is not one of SEQ ID NO: 383, 384, 387, 389, 391, 394, 395, 398, 401, 402, 406, 410, 412, and 421 and an LCVD of SEQ ID NO: 376;
(c) an HCVD of SEQ ID NO: 459, or one of 467-511; and/or an LCVD of SEQ ID NO: 460, or one of 512-541, where the first ABD does not include (i) an HCVD of SEQ ID NO: 459 and an LCVD of SEQ ID NO: 460; (ii) an HCVD of SEQ ID NO: 459 and an LCVD that is not one of SEQ ID NO: 518, 519, 521, 524, 533, and 536; or (iii) an HCVD that is not one of SEQ ID NO: 473, 496, 498, 499, 503, and 508 and an LCVD of SEQ ID NO: 460;
(d) an HCVD of SEQ ID NO: 542, or one of 550-592; and/or an LCVD of SEQ ID NO: 543, or one of 593-627, where the first ABD does not include (i) an HCVD of SEQ ID NO: 542 and an LCVD of SEQ ID NO: 543; (ii) an HCVD of SEQ ID NO: 542 and an LCVD that is not one of SEQ ID NO: 594, 598, 599, 600, 601, 611, 614, and 620; or (iii) an HCVD that is not one of SEQ ID NO: 551, 553, 554, 555, 556, 563, 565, 568, 582, 584, and 585 and an LCVD of SEQ ID NO: 543;
(e) an HCVD of SEQ ID NO: 628, or one of 636-675; and/or an LCVD of SEQ ID NO: 629, or one of 676-709, where the first ABD does not include (i) an HCVD of SEQ ID NO: 628 and an LCVD of SEQ ID NO: 629; (ii) an HCVD of SEQ ID NO: 628 and an LCVD that is not one of SEQ ID NO: 677, 678, 680, 681, 685, 688, 692, 694, 696, 700, 701, 702, 703, 704, and 705; or (iii) an HCVD that is not one of SEQ ID NO: 636, 638, 640, 646, 648, 649, 650, 651, 652, 653, 666, 667, 668, and 669 and an LCVD of SEQ ID NO: 629;
(f) an HCVD of SEQ ID NO: 710, or one of 718-767; and/or an LCVD of SEQ ID NO: 711, or one of 768-802, where the first ABD does not include (i) an HCVD of SEQ ID NO: 710 and an LCVD of SEQ ID NO: 711; (ii) an HCVD of SEQ ID NO: 710 and an LCVD that is not one of SEQ ID NO: 769, 771, 773, 774, 779, 780, 785, 786, 788, 790, 791, 792, 795, and 796; or (iii) an HCVD that is not one of SEQ ID NO: 721, 722, 723, 725, 727, 728, 731, 735, 736, 743, 744, 745, 746, 748, 749, 751, 753, 755, 756, 757, 758, and 761 and an LCVD of SEQ ID NO: 711; and
(g) an HCVD of SEQ ID NO: 803, or one of 811-854; and/or an LCVD of SEQ ID NO: 804, or one of 855-892, where the first ABD does not include (i) an HCVD of SEQ ID NO: 803 and an LCVD of SEQ ID NO: 804; (ii) an HCVD of SEQ ID NO: 803 and an LCVD that is not one of SEQ ID NO: 862, 864-866, 868, 870, 881, and 882; or (iii) an HCVD that is not one of SEQ ID NO: 818, 821, 824, 826-829, 835, 841, 848, and 850 and an LCVD of SEQ ID NO: 804.

In some embodiments, the first antigen-binding domain includes an HCVD of one of: (a) an HCVD including a CDR1, a CDR2, and a CDR3 of SEQ ID NOs: 3, 4, and 5, respectively, with collectively a total of one or more amino acid positions in SEQ ID NOs: 3, 4, and 5 substituted with a histidine; (b) an HCVD including a CDR1, a CDR2, and a CDR3 of SEQ ID NOs: 128, 129, and 130, respectively, with collectively a total of one or more amino acid positions in SEQ ID NOs: 128, 129, and 130 substituted with a histidine; and (c) an HCVD including a CDR1, a CDR2, and a CDR3 of SEQ ID NOs: 231, 232, and 233, respectively, with collectively a total of one or more amino acid positions in SEQ ID NOs: 231, 232, and 233 substituted with a histidine. In some embodiments, the first CCK4-binding domain includes an LCVD of one of: (a) an LCVD including a CDR1, a CDR2, and a CDR3 of SEQ ID NOs: 6, 7, and 8, respectively, with collectively a total of one or more amino acid positions in SEQ ID NOs: 6, 7, and 8 substituted with a histidine; (b) an LCVD including a CDR1, a CDR2, and a CDR3 of SEQ ID NOs: 131, 132, and 133, respectively, with collectively a total of one or more amino acid positions in SEQ ID NOs: 131, 132, and 133 substituted with a histidine; and (c) an LCVD including a CDR1, a CDR2, and a CDR3 of SEQ ID NOs: 234, 235, and 236, respectively, with collectively a total of one or more amino acid positions in SEQ ID NOs: 234, 235, and 236 substituted with a histidine.

In some embodiments, the first CCK4-binding domain includes one of: (a) an HCVD including a CDR1, a CDR2, and a CDR3 of SEQ ID NOs: 3, 4, and 5, respectively, with collectively a total of one or more amino acid positions in SEQ ID NOs: 3, 4, and 5 substituted with a histidine; and/or an LCVD including a CDR1, a CDR2, and a CDR3 of SEQ ID NOs: 6, 7, and 8, respectively, with collectively a total of one or more amino acid positions in SEQ ID NOs: 6, 7, and 8 substituted with a histidine; (b) an HCVD including a CDR1, a CDR2, and a CDR3 of SEQ ID NOs: 128, 129, and 130, respectively, with collectively a total of one or more amino acid positions in SEQ ID NOs: 128, 129, and 130 substituted with a histidine; and/or an LCVD including a CDR1, a CDR2, and a CDR3 of SEQ ID NOs: 131, 132, and 133, respectively, with collectively a total of one or more amino acid positions in SEQ ID NOs: 131, 132, and 133 substituted with a histidine; and (c) an HCVD including a CDR1, a CDR2, and a CDR3 of SEQ ID NOs: 231, 232, and 233, respectively, with collectively a total of one or more amino acid positions in SEQ ID NOs: 231, 232, and 233 substituted with a histidine; and/or an LCVD including a CDR1, a CDR2, and a CDR3 of SEQ ID NOs: 234, 235, and 236, respectively, with collectively a total of one or more amino acid positions in SEQ ID NOs: 234, 235, and 236 substituted with a histidine.

In some embodiments, the first antigen-binding domain includes an HCVD of one of: (a) an HCVD that is at least 90% identical to SEQ ID NO: 1, where the HCVD includes a histidine at one or more positions in SEQ ID NO: 1 selected from the group consisting of: 24, 27, 28, 29, 30, 31, 32, 34, 53, 54, 55, 57, 58, 64, 98, 100, 102, 103, and 107; (b) an HCVD that is at least 90% identical to SEQ ID NO: 126, where the HCVD includes a histidine at one or more positions in SEQ ID NO: 126 selected from the group consisting of: 53, 54, 56, 57, 60, 102, 103, 104, 105, 106, 108, 109, 110, or 111; and (c) an HCVD that is at least 90% identical to SEQ ID NO: 229, where the HCVD includes a histidine at one or more positions in SEQ ID NO: 229 selected from the group consisting of: 27, 32, 33, 34, 35, 51, 54, 56, 58, or 100. In some embodiments, the first antigen-binding domain includes an LCVD of one of: (a) an LCVD that is at least 90% identical to SEQ ID NO: 2, where the LCVD includes a histidine at one or more positions in SEQ ID NO: 2 selected from the group consisting of: 25, 29, 31, 35, 60, 93, or 94; (b) an LCVD that is at least 90% identical to SEQ ID NO: 127, where the LCVD includes a histidine at one or more positions in SEQ ID NO: 127 selected from the group consisting of: 25, 26, 28, 29, 31, 32, 33, 50, 90, 92, and 94; and (c) an LCVD that is at least 90% identical to SEQ ID NO: 230, where the LCVD includes a histidine at one or more positions in SEQ ID NO: 230 selected from the group consisting of: 30, 31, 33, 51, 53, 57, 91, 92, 94, and 99.

In some embodiments, the first antigen-binding domain includes an HCVD of one of: (a) an HCVD that is at least 90% identical to SEQ ID NO: 1, where the HCVD includes a histidine at two or more positions in SEQ ID NO: 1 selected from the group consisting of: 27, 29, 31, 34, and 53; and (b) an HCVD that is at least 90% identical to SEQ ID NO: 126, where the HCVD includes a histidine at two or more positions in SEQ ID NO: 126 selected from the group consisting of: 53, 56, 57, 102, and 105.

In some embodiments, the first antigen-binding domain includes an LCVD of one of: (a) an LCVD that is at least 90% identical to SEQ ID NO: 2, where the LCVD includes a histidine at two or more positions in SEQ ID NO: 2 selected from the group consisting of: 31, 60, and 93; and (b) an LCVD that is at least 90% identical to SEQ ID NO: 127, where the LCVD includes a histidine at two or more positions in SEQ ID NO: 127 from the group consisting of: 28 and 90.

In some embodiments, the first antigen-binding domain includes one of: (a) an HCVD that is at least 90% identical to SEQ ID NO: 1, where the HCVD includes a histidine at one or more positions in SEQ ID NO: 1 selected from the group consisting of: 24, 27, 28, 29, 30, 31, 32, 34, 53, 54, 55, 57, 58, 64, 98, 100, 102, 103, and 107; and/or an LCVD that is at least 90% identical to SEQ ID NO: 2, where the LCVD includes a histidine at one or more positions in SEQ ID NO: 2 selected from the group consisting of: 25, 29, 31, 35, 60, 93, or 94; (b) an HCVD that is at least 90% identical to SEQ ID NO: 126, where the HCVD includes a histidine at one or more positions in SEQ ID NO: 126 selected from the group consisting of: 53, 54, 56, 57, 60, 102, 103, 104, 105, 106, 108, 109, 110, or 111; and/or an LCVD that is at least 90% identical to SEQ ID NO: 127, where the LCVD includes a histidine at one or more positions in SEQ ID NO: 127 selected from the group consisting of: 25, 26, 28, 29, 31, 32, 33, 50, 90, 92, and 94; and (c) an HCVD that is at least 90% identical to SEQ ID NO: 229, where the HCVD includes a histidine at one or more positions in SEQ ID NO: 229 selected from the group consisting of: 27, 32, 33, 34, 35, 51, 54, 56, 58, or 100; and/or an LCVD that is at least 90% identical to SEQ ID NO: 230, where the LCVD includes a histidine at one or more positions in SEQ ID NO: 230 selected from the group consisting of: 30, 31, 33, 51, 53, 57, 91, 92, 94, and 99.

In some embodiments, the first antigen-binding domain includes an HCVD of SEQ ID NO: one of: (a) an HCVD of SEQ ID NO: 1, 14, 17, 18, 19, 20, 21, 22, 24, 25, 29, 30, 31, 33, 34, 40, 44, 46, 48, 49, 53, 86, 87, 88, 89, 90, 91, 92, 93, 94, 95, 96, 98, 99, 100, 101, 102, 103, 104, or 105; (b) an HCVD of SEQ ID NO: 126, 147, 148, 150, 151, 154, 166, 167, 168, 169, 170, 172, 173, 174, 175, 208, 209, 210, 211, 212, 213, 214, 215, 216, 217, 218, 219, 220, 221, 222, 224, 225, 226, or 227; and (c) an HCVD of SEQ ID NO: 229, 238, 243, 244, 245, 246, 248, 251, 253, 255, or 267. In some embodiments, the first antigen-binding domain includes an LCVD of SEQ ID NO: one of: (a) an LCVD of SEQ ID NO: 2, 56, 60, 62, 66, 69, 76, 77, 78, 110, 113, 114, 119, 122, 123, 124, or 125; (b) an LCVD of SEQ ID NO: 127, 181, 182, 184, 185, 187, 188, 189, 191, 199, 201, 203, or 228; and (c) an LCVD of SEQ ID NO: 230, 275, 276, 278, 281, 283, 287, 289, 290, 292, or 297.

In some embodiments, the first antigen-binding domain includes one of: (a) an HCVD of SEQ ID NO: 14, 17, 18, 19, 20, 21, 22, 24, 25, 29, 30, 31, 33, 34, 40, 44, 46, 48, 49, 53, 86, 87, 88, 89, 90, 91, 92, 93, 94, 95, 96, 98, 99, 100, 101, 102, 103, 104, or 105, and/or an LCVD of SEQ ID NO: 2, 56, 60, 62, 66, 69, 76, 77, 78, 110, 113, 114, 119, 122, 123, 124, or 125, where the first antigen binding domain does not include (i) an HCVD of SEQ ID NO: 1 and an LCVD of SEQ ID NO: 2; (ii) an HCVD of SEQ ID NO: 1 and LCVD that is not one of SEQ ID NOs: SEQ ID NOs: 56, 60, 62, 66, 69, 76, 77, 78, 110, 113, 114, 119, and 122-125; or (iii) an LCVD of SEQ ID NO: 2 and an HCVD that is not one of SEQ ID NOs: 14, 17-22, 24, 25, 29-31, 33, 34, 40, 44, 46, 48, 49, 53, 86-96, and 98-105; (b) an HCVD of SEQ ID NO: 147, 148, 150, 151, 154, 166, 167, 168, 169, 170, 172, 173, 174, 175, 208, 209, 210, 211, 212, 213, 214, 215, 216, 217, 218, 219, 220, 221, 222, 224, 225, 226, or 227; and/or an LCVD of SEQ ID NO: 127, 181, 182, 184, 185, 187, 188, 189, 191, 199, 201, 203, or 228, where the first antigen-binding domain does not include (i) an HCVD of SEQ ID NO: 126 and an LCVD of SEQ ID NO: 127; (ii) an HCVD of SEQ ID NO: 126 and an LCVD that is not one of SEQ ID NOs: 181, 182, 184, 185, 187-189, 191, 199, 201, 203, and 228; (iii) an LCVD of SEQ ID NO: 127 and an HCVD that is not one of SEQ ID NOs: 147, 148, 150, 151, 154, 166-170, 172-175, 208-222, and 224-227; and(c) an HCVD of SEQ ID NO: 238, 243, 244, 245, 246, 248, 251, 253, 255, or 267; and/or an LCVD of SEQ ID NO: 230, 275, 276, 278, 281, 283, 287, 289, 290, 292, or 297, where the first antigen-binding domain does not include (i) an HCVD of SEQ ID NO: 229 and an LCVD of SEQ ID NO: 230; (ii) an HCVD of SEQ ID NO: 229 and an LCVD that is not one of SEQ ID NOs: 275, 276, 278, 281, 283, 287, 289, 290, 292, and 297; and (iii) an LCVD of SEQ ID NO: 230 and an HCVD that is not one of SEQ ID NOs: 238, 243-246, 248, 251, 253, 255, and 267.

In some embodiments, a composition including the ABPC provides for an increase in toxin liberation in the target mammalian cell as compared to a composition including the same amount of a control ABPC. In some embodiments, a composition including the ABPC provides for at least a 20% increase in toxin liberation in the target mammalian cell as compared to a composition including the same amount of a control ABPC. In some embodiments, a composition including the ABPC provides for at least a 50% increase in toxin liberation in the target mammalian cell as compared to a composition including the same amount of a control ABPC. In some embodiments, a composition including the ABPC provides for at least a 2-fold increase in toxin liberation in the target mammalian cell as compared to a composition including the same amount of a control ABPC. In some embodiments, a composition including the ABPC provides for at least a 5-fold increase in toxin liberation in the target mammalian cell as compared to a composition including the same amount of a control ABPC.

In some embodiments, a composition including the ABPC provides for an increase in target mammalian cell killing as compared to a composition including the same amount of a control ABPC. In some embodiments, a composition including the ABPC provides for at least a 20% r 50% increase in target mammalian cell killing as compared to a composition including the same amount of a control ABPC. In some embodiments, a composition including the ABPC provides for at least a 2- or 5-fold increase in target mammalian cell killing as compared to a composition including the same amount of a control ABPC.

In some embodiments, a composition including the ABPC provides for an increase in endolysosomal delivery in the target mammalian cell as compared to a composition including the same amount of a control ABPC. In some embodiments, a composition including the ABPC provides for at least a 20% r 50% increase in endolysosomal delivery in the target mammalian cell as compared to a composition including the same amount of a control ABPC. In some embodiments, a composition including the ABPC provides for at least a 2- or 5-fold increase in endolysosomal delivery in the target mammalian cell as compared to a composition including the same amount of a control ABPC.

In some embodiments, a composition including the ABPC results in a less of a reduction in the level of PTK7 presented on the surface of the target mammalian cell as compared to a composition including the same amount of a control ABPC. In some embodiments, a composition including the ABPC does not result in a detectable reduction in the level of the PTK7 presented on the surface of the target mammalian cell. In some embodiments, the target mammalian cell is a cancer cell.

In some embodiments, the dissociation rate of the ABD at a pH of ~4.0 to ~6.5 is at least 10% faster than the dissociation rate of the ABD at a pH of ~7.0 to ~8.0. In some embodiments, the dissociation rate of the ABD at a pH of ~4.0 to ~6.5 is at least 3-fold faster than the dissociation rate of the ABD at a pH of ~7.0 to ~8.0. In some embodiments, the dissociation rate of the ABD at a pH of ~4.0 to ~6.5 is at least 10-fold faster than the dissociation rate of the ABD at a pH of ~7.0 to ~8.0. In some embodiments, the K_{D} of the ABD at a pH of ~4.0 to ~6.5 is at least 10% greater than the K_{D} of the ABD at a pH of ~7.0 to ~8.0. In some embodiments, the K_{D} of the ABD at a pH of ~4.0 to ~6.5 is at least 3-fold greater than the K_{D} of the ABD at a pH of ~7.0 to ~8.0. In some embodiments, the K_{D} of the ABD at a pH of ~4.0 to ~6.5 is at least 10-fold greater than the K_{D} of the ABD at a pH of ~7.0 to ~8.0.

In some embodiments, the ABPC is cytotoxic or cytostatic to the target mammalian cell.

In some embodiments, the ABPC is cross-reactive with a non-human primate PTK7 and human PTK7. In some embodiments, the ABPC is cross-reactive with a non-human primate PTK7, a human PTK7, and one or both of rat PTK7 and a mouse PTK7. In some embodiments, the ABPC is cross-reactive with a non-human primate PTK7, a human PTK7, a rat PTK7, and a mouse PTK7. In some embodiments, the ABD binds to an epitope of PTK7 that is present on the surface of cells from an Old World Monkey.

In some embodiments, the ABPC includes a single polypeptide. In some embodiments, the ABD is selected from a VH domain, a VHH domain, a VNAR domain, and a scFv. In some embodiments, the ABPC is a BiTe, a (scFv)₂, a nanobody, a nanobody-HSA, a DART, a TandAb, a scDiabody, a scDiabody-CH3, scFv-CH-CL-scFv, a HSAbody, scDiabody-HSA, or a tandem-scFv.

In some embodiments, the ABPC includes two or more polypeptides. In some embodiments, the ABPC is selected from the group of an antibody, a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a k-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, and a DEP conjugate.

In some embodiments, at least one polypeptide of the ABPC is conjugated to the toxin, the radioisotope, the drug, or the small molecule via a cleavable linker. In some embodiments, at least one polypeptide of the ABPC is conjugated to the toxin, the radioisotope, the drug, or the small molecule via a non-cleavable linker.

In some embodiments, the half-life of the ABPC in vivo is decreased as compared to the half-life of a control ABPC in vivo. In some embodiments, the ABPC in vivo is decreased about 5% to 95% as compared to the half-life of a control ABPC in vivo. In some embodiments, the half-life of the ABPC in vivo is decreased about 10% to 95% as compared to the half-life of a control ABPC in vivo. In some embodiments, the half-life of the ABPC in vivo is decreased about 30% to 95% as compared to the half-life of a control ABPC in vivo. In some embodiments, the half-life of the ABPC in vivo is decreased about 50% to 95% as compared to the half-life of a control ABPC in vivo. In some embodiments, the half-life of the ABPC in vivo is decreased about 70% to 95% as compared to the half-life of a control ABPC in vivo.

In some embodiments, the control ABPC is capable of specifically binding to PTK7 or an 15 epitope of PTK7 presented on the surface of a target mammalian cell, where: (a) the control ABPC includes a first ABD; (b) the dissociation rate of the first ABD of the control ABPC at a pH of ~4.0 to ~6.5 is no more than 3-fold faster than the dissociation rate at a pH of ~7.0 to ~8.0; and (c) the dissociation constant (K_{D}) of the first ABD of the control ABPC at a pH of ~4.0 to ~6.5 is no more than 3-fold greater than the K_{D} at a pH of ~7.0 to ~8.0.

In some embodiments, the control ABPC is capable of specifically binding to PTK7 or an epitope of PTK7 presented on the surface of a target mammalian cell, where: (a) the control ABPC includes a first ABD; (b) the dissociation rate of the first ABD of the control ABPC at a pH of ~4.0 to ~6.5 is no more than 2-fold faster than the dissociation rate at a pH of ~7.0 to ~8.0; and (c) the dissociation constant (K_{D}) of the first ABD of the ABPC at a pH of ~4.0 to ~6.5 is no more than 2-fold greater than the K_{D} at a pH of ~7.0 to ~8.0.

In some embodiments, the control ABPC is capable of specifically binding to PTK7 or an epitope of PTK7 presented on the surface of a target mammalian cell, where: (a) the control ABPC includes a first ABD; (b) the dissociation rate of the first ABD of the control ABPC at a pH of about 4.0 to about 6.5 is no more than 1-fold faster than the dissociation rate at a pH of 30 about 7.0 to about 8.0; and (c) the dissociation constant (K_{D}) of the first ABD of the control ABPC at a pH of about 4.0 to about 6.5 is no more than 1-fold greater than the K_{D} at a pH of about 7.0 to about 8.0.

In some embodiments, the control ABPC is MYT9345, MYT9359, MYT9361, MYT9412, MYT9460, MYT9792, MYT9797, cofetuzumab, 7C8, or 12C6.

In some embodiments, the ABPC includes a second ABD.

Also provided herein are kits including at least one dose of any ABPC described herein.

Also provided herein are methods of treating a cancer characterized by having a population of cancer cells that have PTK7 or an epitope of PTK7 presented on their surface, the method including: administering a therapeutically effective amount of any of the pharmaceutical compositions described herein or any of the ABPCs described herein to a subject identified as having a cancer characterized by having the population of cancer cells.

Also provided herein are methods of reducing the volume of a tumor in a subject, where the tumor is characterized by having a population of cancer cells that have PTK7 or an epitope of PTK7 presented on their surface, the method including: administering a therapeutically effective amount of any of the pharmaceutical compositions described herein or any of the ABPCs described herein to a subject identified as having a cancer characterized by having the population of cancer cells.

Also provided herein are methods of inducing cell death in a cancer cell in a subject, where the cancer cell has PTK7 or an epitope of PTK7 presented on its surface, where the method includes: administering a therapeutically effective amount of any of the pharmaceutical compositions described herein or any of the ABPCs described herein to a subject identified as having a cancer characterized by having a population of the cancer cells.

In some embodiments, the cancer is a primary tumor. In some embodiments, the cancer is a metastasis. In some embodiments, the cancer is a non-T-cell-infiltrating tumor. In some embodiments, the cancer is a T-cell infiltrating tumor.

Also provided herein are methods of decreasing the risk of developing a metastasis or decreasing the risk of developing an additional metastasis in a subject having a cancer, where the cancer is characterized by having a population of cancer cells that have PTK7 or an epitope of PTK7 presented on their surface the method including: administering a therapeutically effective amount of any one of the pharmaceutical compositions described herein or any of the ABPCs described herein to a subject identified as having a cancer characterized by having the population of cancer cells.

In some embodiments, the cancer is a non-T-cell-infiltrating tumor. In some embodiments, the cancer is a T-cell infiltrating tumor.

As used herein, the term “antigen-binding protein construct” is (i) a single polypeptide that includes at least one ABD or (ii) a complex of two or more polypeptides (e.g., the same or different polypeptides) that together form at least one ABD. Non-limiting examples and aspects of antigen-binding protein constructs are described herein. Additional examples and aspects of antigen-binding protein constructs are known in the art.

A “multi-specific ABPC” is an ABPC that includes two or more different ABDs that collectively specifically bind two or more different epitopes. The two or more different epitopes may be epitopes on the same antigen (e.g., a single polypeptide present on the surface of a cell) or on different antigens (e.g., different proteins present on the surface of the same cell or present on the surface of different cells). In some aspects, the antigen is present on the surface of the cell. In some aspects, a multi-specific ABPC binds two different epitopes (i.e., a “bispecific ABPC”). In some aspects, a multi-specific ABPC binds three different epitopes (i.e., a “trispecific ABPC”). In some aspects, a multi-specific ABPC binds four different epitopes (i.e., a “quadspecific ABPC”). In some aspects, a multi-specific ABPC binds five different epitopes (i.e., a “quintspecific ABPC”). Each binding specificity may be present in any suitable valency. Non-limiting examples of multi-specific ABPC s are described herein.

An “Antigen-Binding Protein” or “ABD” is one or more protein domain(s) (e.g., formed from amino acids from a single polypeptide or formed from amino acids from two or more polypeptides (e.g., the same or different polypeptides) that is capable of specifically binding to one or more different antigen(s). In some examples, an ABD can bind to an antigen or epitope with specificity and affinity similar to that of naturally-occurring antibodies. In some embodiments, the ABD can be an antibody or a fragment thereof. In some embodiments, an ABD can include an alternative scaffold. Non-limiting examples of ABDs are described herein. Additional examples of ABDs are known in the art. In some examples, an ABD can bind to a single antigen.

The term “antibody” is used herein in its broadest sense and includes certain types of immunoglobulin molecules that include one or more ABDs that specifically bind to an antigen or epitope. An antibody specifically includes, e.g., intact antibodies (e.g., intact immunoglobulins, e.g., human IgG (e.g., human IgG1, human IgG2, human IgG3, human IgG4)), antibody fragments, and multi-specific antibodies. One example of an ABD is an ABD formed by a VH-VL dimer. Additional examples of an antibody are described herein. Additional examples of an antibody are known in the art.

The phrase “endosomal/lysosomal pathway” refers to a network of endosomes (early endosomes, multi-vesicular bodies, late endosomes, and lysosomes) in the cytoplasm of a mammalian cell, wherein molecules are internalized through cell-mediated internalization processes, e.g., pinocytosis, micropinocytosis, receptor-mediated endocytosis, and/or phagocytosis, are sorted.

Once the endosomes in the endosomal/lysosomal pathway are purified or isolated, assays for a target protein (e.g., an antigen-binding protein construct described herein) can be performed using methods known in the art (ELISA, Western blot, immunofluorescence, and immunoprecipitation followed by an assay for protein concentration), and can be used to determine the concentration or relative level of the target protein in the endosomes. Alternatively, endosomes in the endosomal/lysosomal pathway can be imaged using immunofluorescence microscopy using an detectably-labelled antibody (e.g., a fluorophore-labelled, a dye-labelled, or a GFP-labelled antibody, e.g., CellLight™ Early Endosome-GFP) that specifically binds to a characteristic protein present in the endosomes (e.g., EEA1 for early endosomes) and a fluorophore-labelled antibody that specifically binds to the protein of interest (e.g., an ABPC), and the level of the target protein in the endosomes can be determined by quantitation of the overlap in the fluorescence emissions of the two different antibodies.

The phrase “endolysosomal delivery” refers to rate of accumulation over time or the total accumulation at a specific timepoint of an ABPC (e.g., any of the ABPCs described herein) in the endosomal/lysosomal pathway in a mammalian cell (e.g., any of the exemplary target mammalian cells described herein).

An exemplary method to calculate the increase in endolysosomal delivery of a pH engineered ABPC variant as compared to its corresponding starting ABPC from cellular fluorescence data is to measure the ratio of the variant's mean fluorescence intensity minus the mean fluorescence intensity of a non-binding IgG control, then all divided by the variant's corresponding starting ABPC's mean fluorescence intensity minus the mean fluorescence intensity of the IgG control.

An exemplary assay for measuring endolysosomal delivery of any of the ABPCs described herein include those which involve labeling of an ABPC with a fluorescent dye, followed by incubation of the labeled ABPC with cells and measurement of cellular fluorescence as an indicator of endolysosomal delivery of the ABPC (e.g., as described generally in Wustner, Traffic 7(6):699-715, 2006). Alternatively, pH-sensitive dyes which preferentially fluoresce at acidic pH but not neutral pH can be used to label any of the ABPCs described herein, which can then be incubated with cells and the cellular fluorescence measured as an indicator of delivery of the ABPC into acidic endolysosomal compartments.

The term “population” when used before a noun means two or more of the specific noun. For example, the phrase “a population of cancer cells” means “two or more cancer cells.” Non-limiting examples of cancer cells are described herein.

The phrase “cytostatic to a cell” refers to a direct or indirect decrease in the proliferation (cell division) of the cell (e.g., a cancer cell) in vivo or in vitro. When an agent is cytostatic to a cell, the agent can, e.g., directly or indirectly result in cell cycle arrest of the cell (e.g., a cancer cell). In some examples, an agent that is cytostatic to a cell can reduce the number of cells in a population of the cells that are in S phase (as compared to the number of cells in a population of the cells that are in S phase prior to contact with the agent). In some examples, an agent that is cytostatic to a cell can reduce the percentage of the cells in S phase by at least 20%, at least 40%, at least 60%, or at least 80% (e.g., as compared to the percentage of cells in a population of the cells that are in S phase prior to contact with the agent).

The phrase “cytotoxic to a cell” refers to the inducement, directly or indirectly, in the death (e.g., necrosis or apoptosis) of the cell (e.g., a mammalian cell, e.g., a cancer cell).

“Affinity” refers to the strength of the sum total of non-covalent interactions between an antigen-binding site and its binding partner (e.g., an antigen or epitope). Unless indicated otherwise, as used herein, “affinity” refers to intrinsic binding affinity, which reflects a 1:1 interaction between members of an ABD and an antigen or epitope. The affinity of a molecule X for its partner Y can be represented by the dissociation equilibrium constant (K_{D}). Affinity can be measured by common methods known in the art, including those described herein. Affinity can be determined, e.g., using surface plasmon resonance (SPR) technology (e.g., BIACORE®) or biolayer interferometry (e.g., FORTEBIO©). Additional methods for determining the affinity for an ABD and its corresponding antigen or epitope are known in the art.

The term “epitope” means a portion of an antigen that is specifically bound by an ABD through a set of physical interactions between: (i) all monomers (e.g. individual amino acid residues, sugar side chains, and post-translationally modified amino acid residues) on the portion of the ABD that specifically binds the antigen and (ii) all monomers (e.g. individual amino acid residues, sugar side chains, post-translationally modified amino acid residues) on the portion of the antigen that is specifically bound by the ABD. Epitopes can, e.g., consist of surface-accessible amino acid residues, sugar side chains, phosphorylated amino acid residues, methylated amino acid residues, and/or acetylated amino acid residues and may have specific three-dimensional structural characteristics, as well as specific charge characteristics. Conformational and non-conformational epitopes are distinguished in that binding to the former, but not the latter, may be lost in the presence of denaturing solvents. In some embodiments, an epitope is defined by a linear amino acid sequence of at least about 3 to 6 amino acids, or about 10 to 15 amino acids. In some embodiments, an epitope refers to a portion of a full-length protein or a portion thereof that is defined by a three-dimensional structure (e.g., protein folding). In some embodiments, an epitope is defined by a discontinuous amino acid sequence that is brought together via protein folding. In some embodiments, an epitope is defined by a discontinuous amino acid sequence that is brought together by quaternary structure (e.g., a cleft formed by the interaction of two different polypeptide chains). The amino acid sequences between the residues that define the epitope may not be critical to three-dimensional structure of the epitope. A conformational epitope may be determined and screened using assays that compare binding of antigen-binding protein construct to a denatured version of the antigen, such that a linear epitope is generated. An epitope may include amino acid residues that are directly involved in the binding, and others, which are not directly involved in the binding.

Methods for identifying an epitope to which an ABD specifically binds are known in the art, e.g., structure-based analysis (e.g. X-ray crystallography, NMR, and/or electron microscopy) (e.g. on the antigen and/or the antigen-ABD complex) and/or mutagenesis-based analysis (e.g. alanine scanning mutagenesis, glycine scanning mutagenesis, and homology scanning mutagenesis) wherein mutants are measured in a binding assay with a binding partner, many of which are known in the art.

The term “paratope” means a portion of an ABD that specifically binds to an antigen through a set of physical interactions between: (i) all monomers (e.g. individual amino acid residues, sugar side chains, posttranslationally modified amino acid residues) on the portion of the ABD that specifically binds the antigen and (ii) all monomers (e.g. individual amino acid residues, sugar side chains, posttranslationally modified amino acid residues) on the portion of the antigen that is specifically bound by the ABD. Paratopes can, e.g., consist of surface-accessible amino acid residues and may have specific three-dimensional structural characteristics, as well as specific charge characteristics. In some embodiments, a paratope refers to a portion of a full-length ABD or a portion thereof that is defined by a three-dimensional structure (e.g., protein folding). In some embodiments, a paratope is defined by a discontinuous amino acid sequence that is brought together via protein folding. In some embodiments, an epitope is defined by a discontinuous amino acid sequence that is brought together by quaternary structure (e.g., a cleft formed by the interaction of two different polypeptide chains). The amino acid sequences between the residues that define the paratope may not be critical to three-dimensional structure of the paratope. A paratope may comprise amino acid residues that are directly involved in the binding, and others, which are not directly involved in the binding.

Methods for identifying a paratope to which an ABD specifically binds are known in the art, e.g., structure-based analysis (e.g., X-ray crystallography, NMR, and/or electron microscopy) (e.g. on the ABD, and/or the ABD-antigen complex), and/or mutagenesis-based analysis (e.g., alanine scanning mutagenesis, glycine scanning mutagenesis, and homology scanning mutagenesis) wherein mutants are measured in a binding assay with a binding partner, many of which are known in the art.

The phrase “present on the surface of a mammalian cell” means (1) an antigen that physically attached to or at least partially embedded in the plasma membrane of a mammalian cell (e.g., a transmembrane protein, a peripheral membrane protein, a lipid-anchored protein (e.g., a GPI-anchor), an N-myristoylated protein, or a S-palmitoylated protein) or (2) an antigen that is stably bound to its cognate receptor, where the cognate receptor is physically attached to the plasma membrane of a cell (e.g., a ligand bound to its cognate receptor, where the cognate receptor is physically attached to the plasma membrane). Non-limiting methods for determining the presence of antigen on the surface of a mammalian cell include fluorescence-activated cell sorting (FACS), immunohistochemistry, cell-fractionation assays and Western blotting.

The phrase “control ABPC” or “control antigen-binding protein construct” means any one of the following: (1) an ABPC that is capable of specifically binding to PTK7 or an epitope of PTK7 presented on the surface of a mammalian cell (e.g., a target mammalian cell), where one or both of the following is true: (a) the dissociation rate of the first ABD at a pH of ~4.0 to ~6.5 (e.g., any of the subranges of this range described herein) is ≤3-fold (e.g., ≤2.8-fold, ≤2.6-fold, ≤2.5-fold, ≤2.4-fold, ≤2.2-fold, ≤2.0-fold, ≤1.8-fold, ≤1.6-fold, ≤1.5-fold, ≤1.4-fold, ≤1.2-fold, ≤1.0-fold, ≤0.8-fold, ≤0.6-fold, ≤0.5-fold, ≤0.4-fold, ≤03-fold 0.2-fold, or ≤0.1-fold) faster than the dissociation rate at a pH of ~7.0 to ~8.0 (e.g., any of the subranges of this range described herein); or (b) the dissociation constant (K_{D}) of the first ABD at a pH of ~4.0 to ~6.5 (e.g., any of the subranges of this range described herein) is ≤3-fold (e.g., ≤2.8-fold, ≤2.6-fold, ≤2.5-fold, ≤2.4-fold, ≤2.2-fold, ≤2.0-fold, ≤1.8-fold, ≤1.6-fold, ≤1.5-fold, ≤1.4-fold, ≤1.2-fold, ≤1.0-fold, ≤0.8-fold, ≤0.6-fold, ≤0.5-fold, ≤0.4-fold, ≤0.3-fold ≤0.2-fold, or ≤0.1-fold) greater than the K_{D} at a pH of ~7.0 to ~8.0 (e.g., any of the subranges of this range described herein); (2) MYT9345; (3) MYT9359; (4) MYT9361; (5) MYT9412; (6) MYT9460; (7) MYT9792; (8) MYT9797; (9) cofetuzumab; (10) 7C8; or (11) 12C6.

The term “extracellular space” means the liquid exterior to the plasma membrane of a mammalian cell. When a mammalian cell is in vitro, the extracellular space can be a liquid culture medium. When a mammalian cell is in vivo, the extracellular space can be, e.g., plasma, serum, blood, interstitial fluid, or lymph.

The term “endolysosomal space” means the fluid encapsulated by the vesicles and organelles that make-up the endosomal/lysosomal pathway in a mammalian cell.

The phrase “a reduced level” or “a decreased level” can be a reduction or decrease of at least a 1% (e.g., ≥2%, ≥4%, ≥6%, ≥8%, ≥10%, ≥12%, ≥14%, ≥16%, ≥18%, ≥20%, ≥22%, ≥24%, ≥26%, ≥30%, ≥35%, ≥40%, ≥45%, ≥50%, ≥55%, ≥60%, ≥65%, ≥70%, ≥75%, ≥80%, ≥85%, ≥90%, ≥95%, or ≥99%) reduction as compared to a reference level or value.

The term “cell killing potency” refers to the ability of an agent (e.g., any of the ABPCs described herein) to induce, directly or indirectly, the apoptosis and/or necrosis of a mammalian cell (e.g., a cancer cell), measured as a rate over time or at a relevant timepoint. Methods for determining the cell killing potency of a cell are known in the art (e.g., trypan blue staining, microscopy, fluorescence-assisted cell sorting, and assays to detect markers of apoptosis (e.g., Annexin V)). In non-limiting examples, cell killing potency can be measured, e.g., by cell killing at a single concentration of an agent, by the IC50 of the agent (i.e. the concentration of the agent whereby half the maximal cell killing potency is achieved), or by the ratio of an agent's dissociation constant KD on mammalian cells divided by its IC50. In some non-limiting examples, the IC50s and/or the KD ratios described herein are compared to those of a control ABPC (as defined herein), and, optionally, demonstrate that the ABPCs described herein have a higher cell killing potency as compared to the control ABPC.

The term “toxin liberation” refers to the ability of a mammalian cell (e.g., a non-cancerous mammalian cell or a cancer cell) to internalize (e.g., via pinocytosis and/or receptor-mediated endocytosis) any of the ABPCs described herein (e.g., any of ABPCs or control ABPCs described herein) that are conjugated to a toxin, and subsequently release the toxin conjugated to the ABPC, measured as a rate over time or at a specific timepoint. Toxin liberation can be assessed using a variety of different exemplary assays, e.g., ELISA, immunofluorescence, cell killing assays, cell cycle arrest assays, DNA damage assays, mass spectrometry, HPLC, and/or an isotope-labeled toxin.

The phrase “target cell” or “target mammalian cell” or “mammalian target cell” means a mammalian cell that has at least one PTK7 present on its surface. In some examples, a mammalian target cell can be a cancer cell. In some embodiments of a target mammalian cell can have a total of about the following (each: about 10%): 1-10E6, 1-9E6, 1-8E6, 1-7E6, 1-6E6, 1-5E6, 1-4E6, 1-3E6, 1-2E6, 1-1E6, 1-800,000, 1-600,000, 1-400,000, 1-200,000, 1-100,000, 1-80,000, 1-80,000, 1-75,000, 1-70,000, 1-65,000, 1-60,000, 1-55,000, 1-50,000, 1-45,000, 1-40,000, 1-35,000, 1-30,000, 1-25,000, 1-20,000, 1-15,000, 1-10,000, 1-7,500, 1-5,000, 1-4,000, 1-3,000, 1-2,000, 1-1,000, 1-500, 1-100, 1-50, or 1-10, or any of the ranges of numbers recited in of US 2022/0281984, of the PTK7 present on the plasma membrane of the target mammalian cell.

The phrase “antigen density” means the number of PTK7 present on the surface of a target mammalian cell or the average number of PTK7 on the surface of a population of particular type of target mammalian cells. It can be measured, e.g., using the Quantibright bead kit or radiolabel (e.g., BD Biosciences PE Phycoerythrin Fluorescence Quantitation Kit, catalog #340495).

The phrase “amino acid substituted with a histidine” means the substitution of an amino acid residue that is not histidine in a reference polypeptide sequence with a histidine. Non-limiting methods for substituting an amino acid residue in a reference polypeptide with a histidine are described herein. Additional methods for substituting an amino acid residue in a reference polypeptide with a histidine are known in the art.

The phrase “amino acid substituted with an alanine” means the substitution of an amino acid residue that is a histidine in a reference polypeptide sequence with an alanine. Non-limiting methods for substituting a histidine in a reference polypeptide with an alanine are described herein. Additional methods for substituting a histidine in a reference polypeptide with an alanine are known in the art.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1: SDS PAGE for Cofetuzunmab histidine scanning and alanine scanning. Expi293 cell culture supernatants post-harvest were loaded on non-reduced SDS PAGE gels to confirm expression of Cofetuzurnab and histidine scanning and alanine scanning variants. Arrows show the corresponding size for an IgG on a non-reduced SDS PAGE gel. MYT6002 is Cofetuzumab and MYT6152-MYT6193 are Cofetuzumab heavy chain histidine scanning and alanine scanning variants.

FIGS. 2a to 2aq: Binding of Cofetuzumab starting ABPC and histidine scanning and alanine scanning variants to PTK7 by biolayer interferometry. MYT6002 (Cofetuzumab) and MYT6152 MYT6193, heavy chain histidine scanning and alanine scanning 30 variants, were captured on anti-human Fe biosensors and associated with PTK7 at pH 7.4. Dissociation was at pH 7.4 (black trace) or pH 5.4 (grey trace).

FIG. 3: Construct identifier to SEQ TD NO correspondence table. Constructs, heavy chain histidine scanning and alanine scanning variants, are listed in the first column of the table, SEQ ID NOs are listed and correspond to constructs on the left and the appropriate heavy chain and/or light chain categories along the top.

FIG. 4: SDS PAGE for Cofetazunab histidine scanning and alanine scanning. Expi293 cell culture supernatants post-harvest were loaded on non-reduced SDS PAGE gels to confirm expression of Cofetuzumab histidine scanning and alanine scanning variants. Arrows show the corresponding size for an IgG on a non-reduced SDS PAGE gel MYT6194-MYT6224 are Cofetuzumab light chain histidine scanning and alanine scanning variants.

FIGS. 5a to 5af: Binding of Cofetuzumab starting ABPC and histidine scanning and alanine scanning variants to PTK7 by biolayer interferometry. MYT6002 (Cofetuzumab) and MYT6194-MYT6224, light chain histidine scanning and alanine scanning variants of Cofetuzumab, were captured on anti-human Fe biosensors and associated with PTK7 at pH 7.4 Dissociation was at pH 7.4 (black trace) or pH 5.4 (grey trace),

FIG. 6: Construct identifier to SEQ ID NO correspondence table. Constructs, light chain histidine scanning and alanine scanning variants, are listed in the first column of the table, SEQ ID NOs are listed and correspond to constructs on the left and the appropriate heavy chain and/or light chain categories along the top.

FIG. 7: SDS PAGE for Cofetuzumab histidine scanning and alanine scanning. Expi293 cell culture supernatants post-harvest were loaded on non-reduced SDS PAGE gels to confirm expression of Cofetuzumab histidine scanning and alanine scanning variants. Arrows show the corresponding size for an IgG on a non-reduced SDS PAGE gel. MYT7837-MYT7856 are Cofetuzumab heavy chain combinations histidine scanning and alanine scanning variants.

FIGS. 8a to 8u: Binding of histidine scanning and alanine scanning variants of Cofetuzumab to PTK7 by biolayer interferometry. MYT6002 (Cofetuzumab) and MYT7837-MYT7856, heavy chain combination histidine scanning and alanine scanning variants of Cofetuzumab, were captured on anti-human Fc biosensors and associated with PTK7 at pH 7.4. Dissociation was at pH 7.4 (black trace) or pH 5.4 (grey trace).

FIG. 9: Construct identifier to SEQ ID NO correspondence table. Constructs, heavy chain combinations histidine scanning and alanine scanning variants, are listed in the first column of the table, SEQ ID NOs are listed and correspond to constructs on the left and the appropriate heavy chain and/or light chain categories along the top.

FIG. 10: SDS PAGE for Cofetuzunmab histidine scanning and alanine scanning. Expi293 cell culture supernatants post-harvest were loaded on non-reduced SDS PAGE gels to confirm expression of Cofetuzumab histidine scanning and alanine scanning variants. Arrows show the corresponding size for an IgG on a non-reduced SDS PAGE gel. MYT7857-MYT7876 are Cofetuzurnab light chain combinations histidine scanning and alanine scanning variants.

FIGS. 11a to 11u: Binding of histidine scanning and alanine scanning variants of Cofetuzunmab to PTK7 by biolayer interferometry. MYT6002 (Cofetuzumab) and MYT7857 MYT7876, light chain combination histidine scanning and alanine scanning variants of Cofetuzumab, were captured on anti-human Fc biosensors and associated with PTK7 at pH 7.4. Dissociation was at pH 7.4 (black trace) or pH 5.4 (grey trace).

FIG. 12: Construct identifier to SEQ ID NO correspondence table. Constructs, light chain combinations histidine scanning and alanine scanning variants, are listed in the first column of the table, SEQ ID NOs are listed and correspond to constructs on the left and the appropriate heavy chain and/or light chain categories along the top.

FIG. 13: SDS PAGE for 7C8 histidine scanning and alanine scanning. Expi293 cell culture supernatants post-harvest were loaded on non-reduced SDS PAGE gels to confirm expression of 7C8 and histidine scanning and alanine scanning variants. Arrows show the corresponding size for an IgG on a non-reduced SDS PAGE gel. MYT6003 is 7C8 and the rest of the lanes (MYT6225-MYT6270) are 7C8 heavy chain histidine scanning and alanine scanning variants.

FIGS. 14a to 14au: Binding of 7C8 starting ABPC and histidine scanning and alanine scanning variants to PTK7 by biolayer interferometry. MYT6003 (7C8) and MYT6225-MYT6270, heavy chain histidine scanning and alanine scanning variants, were captured on anti-human Fc biosensors and associated with PTK7 at pH 7.4. Dissociation was at pH 7.4 (black trace) or pH 5.4 (grey trace).

FIG. 15: Construct identifier to SEQ ID NO correspondence table. Constructs, heavy chain histidine scanning and alanine scanning variants, are listed in the first column of the table, SEQ ID NOs are listed and correspond to constructs on the left and the appropriate heavy chain and/or light chain categories along the top.

FIG. 16: SDS PAGE for 7C8 histidine scanning and alanine scanning. Expi293 cell culture supernatants post-harvest were loaded on non-reduced SDS PAGE gels to confirm expression of 7C8 histidine scanning and alanine scanning van ants. Arrows show the corresponding size for an IgG on a non-reduced SDS PAGE gel. MYT6271-MYT6298 are 7C8 light chain histidine scanning and alanine scanning variants.

FIGS. 17a to 17ac: Binding of 7C8 starting ABPC and histidine scanning and alanine scanning variants to PTK7 by biolayer interferometry. MYT6003 (7C8) and MYT6271 MYT6298, light chain histidine scanning and alanine scanning variants of 7C8, were captured on anti-human Fe biosensors and associated with PTK7 at pH 7.4. Dissociation was at pH 7.4 (black trace) or pH 5.4 (grey trace).

FIG. 18: Construct identifier to SEQ ID NO correspondence table. Constructs, light chain histidine scanning and alanine scanning variants, are listed in the first column of the table, SEQ ID NOs are listed and correspond to constructs on the left and the appropriate heavy chain and/or light chain categories along the top.

FIG. 19: SDS PAGE for 7C8 histidine scanning and alanine scanning. Expi293 cell culture supernatants post-harvest were loaded on non-reduced SDS PAGE gels to confirm expression of 7C8 histidine scanning and alanine scanning variants. Arrows show the corresponding size for an IgG on a non-reduced SDS PAGE gel MYT7816-MYT7835 are 7C8 heavy chain combinations histidine scanning and alanine scanning variants.

FIGS. 20a to 20u: Binding of histidine scanning and alanine scanning variants of 7C8 to PTK7 by biolayer interferometry. MYT6003 (7C8) and MYT7816-MYT7835, heavy chain combination histidine scanning and alanine scanning variants of 7C8, were captured on anti-human Fc biosensors and associated with PTK7 at pH 7.4. Dissociation was at pH 7.4 (black trace) or pH 5.4 (grey trace).

FIG. 21: Construct identifier to SEQ ID NO correspondence table. Constructs, heavy chain combinations histidine scanning and alanine scanning variants, are listed in the first column of the table, SEQ ID NOs are listed and correspond to constructs on the left and the appropriate heavy chain and/or light chain categories along the top.

FIG. 22: SDS PAGE for 7C8 histidine scanning and alanine scanning. Expi293 cell culture supernatants post-harvest were loaded on non-reduced SDS PAGE gels to confirm expression of 7C8 histidine scanning and alanine scanning variants, Arrows show the corresponding size for an IgG on a non-reduced SDS PAGE gel. MYT7836 is a 7C8 light chain combination histidine scanning and alanine scanning variant.

FIGS. 23a to 23b: Binding of histidine scanning and alanine scanning variants of 7C8 to PTK7 by biolayer interferometry. MYT6003 (7C8) and MYT7836, a light chain combination histidine scanning and alanine scanning variant of 7C8, were captured on anti-human Fe biosensors and associated with PTK7 at pH 7.4. Dissociation was at pH 7.4 (black trace) or pH 5.4 (grey trace).

FIG. 24: Construct identifier to SEQ ID NO correspondence table. Constructs, light chain combinations histidine scanning and alanine scanning variants, are listed in the first column of the table, SEQ ID NOs are listed and correspond to constructs on the left and the appropriate heavy chain and/or light chain categories along the top.

FIG. 25: SDS PAGE for 12C6 histidine scanning and alanine scanning. Expi293 cell culture supernatants post-harvest were loaded on non-reduced SDS PAGE gels to confirm expression of 12C6 and histidine scanning and alanine scanning variants. Arrows show the corresponding size for an IgG on a non-reduced SDS PAGE gel. MYT7959 is 12C6 and the rest of the lanes (MYT7960-MYT7991) are 12C6 heavy chain histidine scanning and alanine scanning variants.

FIGS. 26a to 26ag: Binding of 12C6 starting ABPC and histidine scanning and alanine scanning variants to PTK7 by biolayer interferometry. MYT7959 (12C6) and MYT7960-MYT7991, heavy chain histidine scanning and alanine scanning variants, were captured on anti-human Fe biosensors and associated with PTK7 at pH 7.4. Dissociation was at pH 7.4 (black trace) or pH 5.4 (grey trace).

FIG. 27: Construct identifier to SEQ ID NO correspondence table. Constructs, heavy chain histidine scanning and alanine scanning variants, are listed in the first column of the table, SEQ ID NOs are listed and correspond to constructs on the left and the appropriate heavy chain and/or light chain categories along the top.

FIG. 28: SDS PAGE for 12C6 histidine scanning and alanine scanning. Expi293 cell culture supernatants post-harvest were loaded on non-reduced SDS PAGE gels to confirm expression of 12C6 histidine scanning and alanine scanning variants. Arrows show the corresponding size for an IgG on a non-reduced SDS PAGE gel. MYT7992-MYT8020 are 12C6 light chain histidine scanning and alanine scanning variants.

FIGS. 29a to 29ad: Binding of 12C6 starting ABPC and histidine scanning and alanine scanning variants to PTK7 by biolayer interferometry. MYT7959 (12C6) and MYT7992-MYT8020, light chain histidine scanning and alanine scanning variants of 12C6, were captured on anti-human Fc biosensors and associated with PTK7 at pH 7.4. Dissociation was at pH 7.4 (black trace) or pH 5.4 (grey trace).

FIG. 30: Construct identifier to SEQ ID NO correspondence table. Constructs, light chain histidine scanning and alanine scanning variants, are listed in the first column of the table, SEQ ID NOs are listed and correspond to constructs on the left and the appropriate heavy chain and/or light chain categories along the top.

FIGS. 31a to 31d: Internalization of anti-PTK7 mAbs in cells. Anti-PTK7 pH engineered antibody variants, corresponding starting ABPC antibody, control IgG1 isotype control (BP0297, Bioxcell), along with a vehicle control, as specified in FIG. 31 were assayed for internalization and endolysosomal delivery as measured by median fluorescence intensity on cells, at 25 nM after 24 hours. Error bars represent standard deviation where present. Numbers above the bars represent fold change over the corresponding starting ABPC.

FIGS. 32a to 31b: Internalization of anti-PTK7 mAbs in cells. Anti-PTK7 pH engineered antibody variants, corresponding starting ABPC antibody, control IgG1 isotype control (BP0297, Bioxcell), along with a vehicle control, as specified in FIG. 32 were assayed for internalization and endolysosomal delivery as measured by median fluorescence intensity on cells, at 25 nM after 24 hours. Error bars represent standard deviation where present. Numbers above the bars represent fold change over the corresponding starting ABPC.

FIGS. 33a to 33b: Internalization of anti-PTK7 mAbs in cells. Anti-PTK7 pH engineered antibody variants, corresponding starting ABPC antibody, control IgG1 isotype control (BP0297, Bioxcell), along with a vehicle control, as specified in FIG. 33 were assayed for internalization and endolysosomal delivery as measured by median fluorescence intensity on cells, at 25 nM after 24 hours. Error bars represent standard deviation where present. Numbers above the bars represent fold change over the corresponding starting ABPC.

FIGS. 34a to 34b: Presents octet plots for MYT9345 HC and LC variants. For each of FIGS. 34-40, the y-axis (nm) array of plots was either (A) fixed or (B) automatically set by the software to maximize the vertical spread of the data. For each fixed y-axis, the major tick is 0.4 nm and the minor tick is 0.1 nm.

FIGS. 35a to 35b: Presents octet plots for MYT9359 HC and LC variants.

FIGS. 36a to 36b: Presents octet plots for MYT9361 HC and LC variants.

FIGS. 37a to 37b: Presents octet plots for MYT9412 HC and LC variants.

FIGS. 38a to 38b: Presents octet plots for MYT9460 HC and LC variants.

FIGS. 39a to 39b: Presents octet plots for MYT9792 HC and LC variants.

FIGS. 40a to 40b: Presents octet plots for MYT9797 HC and LC variants.

### DETAILED DESCRIPTION

Provided herein are antigen-binding protein constructs (ABPCs) that include: a first ABD (ABD) that is capable of specifically binding PTK7 or an epitope of PTK7 presented on the surface of a target mammalian cell, where: (a) the dissociation rate of the first ABD at a pH of about 4.0 to about 6.5 is faster than the dissociation rate at a pH of about 7.0 to about 8.0; and/or (b) the dissociation constant (K_{D}) of the first ABD at a pH of about 4.0 to about 6.5 is greater than the K_{D} at a pH of about 7.0 to about 8.0. In some examples of these ABPCs, the ABPC is degraded in the target mammalian cell following internalization of the ABPC by the target mammalian cell. Some examples of any of the ABPCs described herein can further include a conjugated toxin, radioisotope, drug, or small molecule (e.g., a fluorophore or dye).

Also provided are antigen-binding protein constructs (ABPCs) that include: a first ABD that is capable of specifically binding PTK7 or an epitope of PTK7 presented on the surface of a target mammalian cell; and a conjugated toxin, radioisotope, drug, or small molecule, where: (a) the dissociation rate of the first ABD at a pH of about 4.0 to about 6.5 is faster than the dissociation rate at a pH of about 7.0 to about 8.0; and/or the dissociation constant (K_{D}) of the first ABD at a pH of about 4.0 to about 6.5 is greater than the K_{D} at a pH of about 7.0 to about 8.0; and (b) a composition including the ABPC provides for one or more (e.g., two or three) of: an increase (e.g., a detectable increase) in toxin liberation in the target mammalian cell as compared to a composition comprising the same amount of a control ABPC; an increase (e.g., a detectable increase) in target mammalian cell killing as compared to a composition comprising the same amount of a control ABPC; and an increase (e.g., a detectable increase) in endolysosomal delivery in the target mammalian cell as compared to a composition comprising the same amount of a control ABPC.

In some examples of any of the ABPCs described herein, the first ABD includes an HCVD of cofetuzumab with one or more amino acids substituted with a histidine. In some examples of any of the ABPCs described herein, the first ABD includes an LCVD of cofetuzumab with one or more amino acids substituted with a histidine. In some examples of any of the ABPCs described herein, the first ABD includes an HCVD of cofetuzumab with one or more amino acids substituted with a histidine; and an LCVD of cofetuzumab with one or more amino acids substituted with a histidine. In some examples of any of the ABPCs described herein, the HCVD of cofetuzumab comprises SEQ ID NO: 1. In some examples of any of the ABPCs described herein, the LCVD of cofetuzumab comprises SEQ ID NO: 2.

In some examples of any of the ABPCs described herein, the first ABD comprises an HCVD comprising a CDR1, a CDR2, and a CDR3 of SEQ ID NO: 3, SEQ ID NO: 4, and SEQ ID NO: 5, respectively, with collectively a total of one or more (e.g., one, two, three, four, five, six, seven, eight, nine, or ten) amino acid positions in SEQ ID NOs: 3, 4, and 5 substituted with a histidine. In some examples of any of the ABPCs described herein, the first ABD comprises an LCVD comprising a CDR1, a CDR2, and a CDR3 of SEQ ID NO: 6, SEQ ID NO: 7, and SEQ ID NO: 8, respectively, with collectively a total of one or more (e.g., one, two, three, four, five, six, seven, eight, nine, or ten) amino acid positions in SEQ ID NOs: 6, 7, and 8 substituted with a histidine. In some examples of any of the ABPCs described herein, the first ABD includes: an HCVD comprising a CDR1, a CDR2, and a CDR3 of SEQ ID NO: 3, SEQ ID NO: 4, and SEQ ID NO: 5, respectively, with collectively a total of one or more (e.g., one, two, three, four, five, six, seven, eight, nine, or ten) amino acid positions in SEQ ID NOs: 3, 4, and 5 substituted with a histidine; and an LCVD comprising a CDR1, a CDR2, and a CDR3 of SEQ ID NO: 6, SEQ ID NO: 7, and SEQ ID NO: 8, respectively, with collectively a total of one or more (e.g., one, two, three, four, five, six, seven, eight, nine, or ten) amino acid positions in SEQ ID NOs: 6, 7, and 8 substituted with a histidine.

In some examples of any of the ABPCs described herein, the first ABD includes an HCVD that is at least 90% (e.g., at least 92%, at least 94%, at least 96%, at least 98%, at least 99%, or 100%) identical to SEQ ID NO: 1, where the HCVD includes a histidine at one or more amino acid positions in SEQ ID NO: 1 selected from the group consisting of: 24, 27, 28, 29, 30, 31, 32, 34, 53, 54, 55, 57, 58, 64, 98, 100, 102, 103, and 107. In some examples of any of the ABPCs described herein, the first ABD includes an LCVD that is at least 90% (e.g., at least 92%, at least 94%, at least 96%, at least 98%, at least 99%, or 100%) identical to SEQ ID NO: 2, where the LCVD includes a histidine at one or more amino acid positions in SEQ ID NO: 2 selected from the group consisting of: 25, 29, 31, 35, 60, 93, or 94. In some examples of any of the ABPCs described herein the first ABD includes: an HCVD that is at least 90% (e.g., at least 92%, at least 94%, at least 96%, at least 98%, at least 99%, or 100%) identical to SEQ ID NO: 1, where the HCVD includes a histidine at one or more amino acid positions in SEQ ID NO: 1 selected from the group consisting of: 24, 27, 28, 29, 30, 31, 32, 34, 53, 54, 55, 57, 58, 64, 98, 100, 102, 103, and 107, and an LCVD that is at least 90% (e.g., at least 92%, at least 94%, at least 96%, at least 98%, at least 99%, or 100%) identical to SEQ ID NO: 2, where the LCVD includes a histidine at one or more amino acid positions in SEQ ID NO: 2 selected from the group consisting of: 25, 29, 31, 35, 60, 93, or 94.

In some examples of any of the ABPCs described herein, an HCVD includes an HCVD that is at least 90% (e.g., at least 92%, at least 94%, at least 96%, at least 98%, at least 99%, or 100%) identical to SEQ ID NO: 1, where the HCVD includes a histidine at any of the specific combinations of one or more amino acid positions in SEQ ID NO: 1 listed in Table 1.

In some examples of any of the ABPCs described herein, the first ABD includes an HCVD that is at least 90% (e.g., at least 92%, at least 94%, at least 96%, at least 98%, at least 99%, or 100%) identical to SEQ ID NO: 1, where the HCVD includes an alanine at position 35 in SEQ ID NO: 1.

In some examples of any of the ABPCs described herein the first ABD includes: an HCVD that is at least 90% (e.g., at least 92%, at least 94%, at least 96%, at least 98%, at least 99%, or 100%) identical to SEQ ID NO: 1, where the HCVD includes an alanine at position 35 in SEQ ID NO: 1, and an LCVD that is at least 90% (e.g., at least 92%, at least 94%, at least 96%, at least 98%, at least 99%, or 100%) identical to SEQ ID NO: 2, where the LCVD includes a histidine at one or more amino acid positions in SEQ ID NO: 2 selected from the group consisting of: 25, 29, 31, 35, 60, 93, or 94.

Table 1. Exemplary Combinations of Amino Acid Positions in SEQ ID NO: 1 that can be Substituted with Histidine

TABLE 1
Exemplary Combinations of Amino Acid Positions in SEQ ID NO: 1 that can be Substituted with Histidine

24 + 27, 24 + 28, 24 + 29, 24 + 30, 24 + 31, 24 + 32, 24 + 34, 24 + 53, 24 + 54, 24 + 55, 24 + 57, 24 + 58,
24 + 64, 24 + 98, 24 + 100, 24 + 102, 24 + 103, 24 + 107, 27 + 28, 27 + 29, 27 + 30, 27 + 31, 27 + 32,
27 + 34, 27 + 53, 27 + 54, 27 + 55, 27 + 57, 27 + 58, 27 + 64, 27 + 98, 27 + 100, 27 + 102, 27 + 103,
27 + 107, 28 + 29, 28 + 30, 28 + 31, 28 + 32, 28 + 34, 28 + 53, 28 + 54, 28 + 55, 28 + 57, 28 + 58, 28 + 64,
28 + 98, 28 + 100, 28 + 102, 28 + 103, 28 + 107, 29 + 30, 29 + 31, 29 + 32, 29 + 34, 29 + 53, 29 + 54,
29 + 55, 29 + 57, 29 + 58, 29 + 64, 29 + 98, 29 + 100, 29 + 102, 29 + 103, 29 + 107, 30 + 31, 30 + 32,
30 + 34, 30 + 53, 30 + 54, 30 + 55, 30 + 57, 30 + 58, 30 + 64, 30 + 98, 30 + 100, 30 + 102, 30 + 103,
30 + 107, 31 + 32, 31 + 34, 31 + 53, 31 + 54, 31 + 55, 31 + 57, 31 + 58, 31 + 64, 31 + 98, 31 + 100, 31 + 102,
31 + 103, 31 + 107, 32 + 34, 32 + 53, 32 + 54, 32 + 55, 32 + 57, 32 + 58, 32 + 64, 32 + 98, 32 + 100,
32 + 102, 32 + 103, 32 + 107, 34 + 53, 34 + 54, 34 + 55, 34 + 57, 34 + 58, 34 + 64, 34 + 98, 34 + 100,
34 + 102, 34 + 103, 34 + 107, 53 + 54, 53 + 55, 53 + 57, 53 + 58, 53 + 64, 53 + 98, 53 + 100, 53 + 102,
53 + 103, 53 + 107, 54 + 55, 54 + 57, 54 + 58, 54 + 64, 54 + 98, 54 + 100, 54 + 102, 54 + 103, 54 + 107,
55 + 57, 55 + 58, 55 + 64, 55 + 98, 55 + 100, 55 + 102, 55 + 103, 55 + 107, 57 + 58, 57 + 64, 57 + 98,
57 + 100, 57 + 102, 57 + 103, 57 + 107, 58 + 64, 58 + 98, 58 + 100, 58 + 102, 58 + 103, 58 + 107, 64 + 98,
64 + 100, 64 + 102, 64 + 103, 64 + 107, 98 + 100, 98 + 102, 98 + 103, 98 + 107, 100 + 102, 100 + 103,
100 + 107, 102 + 103, 102 + 107, 103 + 107, 24 + 27 + 28, 24 + 27 + 29, 24 + 27 + 30, 24 + 27 + 31,
24 + 27 + 32, 24 + 27 + 34, 24 + 27 + 53, 24 + 27 + 54, 24 + 27 + 55, 24 + 27 + 57, 24 + 27 + 58, 24 + 27 + 64,
24 + 27 + 98, 24 + 27 + 100, 24 + 27 + 102, 24 + 27 + 103, 24 + 27 + 107, 24 + 28 + 29, 24 + 28 + 30,
24 + 28 + 31, 24 + 28 + 32, 24 + 28 + 34, 24 + 28 + 53, 24 + 28 + 54, 24 + 28 + 55, 24 + 28 + 57, 24 + 28 + 58,
24 + 28 + 64, 24 + 28 + 98, 24 + 28 + 100, 24 + 28 + 102, 24 + 28 + 103, 24 + 28 + 107, 24 + 29 + 30,
24 + 29 + 31, 24 + 29 + 32, 24 + 29 + 34, 24 + 29 + 53, 24 + 29 + 54, 24 + 29 + 55, 24 + 29 + 57, 24 + 29 + 58,
24 + 29 + 64, 24 + 29 + 98, 24 + 29 + 100, 24 + 29 + 102, 24 + 29 + 103, 24 + 29 + 107, 24 + 30 + 31,
24 + 30 + 32, 24 + 30 + 34, 24 + 30 + 53, 24 + 30 + 54, 24 + 30 + 55, 24 + 30 + 57, 24 + 30 + 58, 24 + 30 + 64,
24 + 30 + 98, 24 + 30 + 100, 24 + 30 + 102, 24 + 30 + 103, 24 + 30 + 107, 24 + 31 + 32, 24 + 31 + 34,
24 + 31 + 53, 24 + 31 + 54, 24 + 31 + 55, 24 + 31 + 57, 24 + 31 + 58, 24 + 31 + 64, 24 + 31 + 98, 24 + 31 + 100,
24 + 31 + 102, 24 + 31 + 103, 24 + 31 + 107, 24 + 32 + 34, 24 + 32 + 53, 24 + 32 + 54, 24 + 32 + 55,
24 + 32 + 57, 24 + 32 + 58, 24 + 32 + 64, 24 + 32 + 98, 24 + 32 + 100, 24 + 32 + 102, 24 + 32 + 103,
24 + 32 + 107, 24 + 34 + 53, 24 + 34 + 54, 24 + 34 + 55, 24 + 34 + 57, 24 + 34 + 58, 24 + 34 + 64, 24 + 34 + 98,
24 + 34 + 100, 24 + 34 + 102, 24 + 34 + 103, 24 + 34 + 107, 24 + 53 + 54, 24 + 53 + 55, 24 + 53 + 57,
24 + 53 + 58, 24 + 53 + 64, 24 + 53 + 98, 24 + 53 + 100, 24 + 53 + 102, 24 + 53 + 103, 24 + 53 + 107,
24 + 54 + 55, 24 + 54 + 57, 24 + 54 + 58, 24 + 54 + 64, 24 + 54 + 98, 24 + 54 + 100, 24 + 54 + 102,
24 + 54 + 103, 24 + 54 + 107, 24 + 55 + 57, 24 + 55 + 58, 24 + 55 + 64, 24 + 55 + 98, 24 + 55 + 100,
24 + 55 + 102, 24 + 55 + 103, 24 + 55 + 107, 24 + 57 + 58, 24 + 57 + 64, 24 + 57 + 98, 24 + 57 + 100,
24 + 57 + 102, 24 + 57 + 103, 24 + 57 + 107, 24 + 58 + 64, 24 + 58 + 98, 24 + 58 + 100, 24 + 58 + 102,
24 + 58 + 103, 24 + 58 + 107, 24 + 64 + 98, 24 + 64 + 100, 24 + 64 + 102, 24 + 64 + 103, 24 + 64 + 107,
24 + 98 + 100, 24 + 98 + 102, 24 + 98 + 103, 24 + 98 + 107, 24 + 100 + 102, 24 + 100 + 103, 24 + 100 + 107,
24 + 102 + 103, 24 + 102 + 107, 24 + 103 + 107, 27 + 28 + 29, 27 + 28 + 30, 27 + 28 + 31, 27 + 28 + 32,
27 + 28 + 34, 27 + 28 + 53, 27 + 28 + 54, 27 + 28 + 55, 27 + 28 + 57, 27 + 28 + 58, 27 + 28 + 64, 27 + 28 + 98,
27 + 28 + 100, 27 + 28 + 102, 27 + 28 + 103, 27 + 28 + 107, 27 + 29 + 30, 27 + 29 + 31, 27 + 29 + 32,
27 + 29 + 34, 27 + 29 + 53, 27 + 29 + 54, 27 + 29 + 55, 27 + 29 + 57, 27 + 29 + 58, 27 + 29 + 64, 27 + 29 + 98,
27 + 29 + 100, 27 + 29 + 102, 27 + 29 + 103, 27 + 29 + 107, 27 + 30 + 31, 27 + 30 + 32, 27 + 30 + 34,
27 + 30 + 53, 27 + 30 + 54, 27 + 30 + 55, 27 + 30 + 57, 27 + 30 + 58, 27 + 30 + 64, 27 + 30 + 98, 27 + 30 + 100,
27 + 30 + 102, 27 + 30 + 103, 27 + 30 + 107, 27 + 31 + 32, 27 + 31 + 34, 27 + 31 + 53, 27 + 31 + 54,
27 + 31 + 55, 27 + 31 + 57, 27 + 31 + 58, 27 + 31 + 64, 27 + 31 + 98, 27 + 31 + 100, 27 + 31 + 102,
27 + 31 + 103, 27 + 31 + 107, 27 + 32 + 34, 27 + 32 + 53, 27 + 32 + 54, 27 + 32 + 55, 27 + 32 + 57, 27 + 32 + 58,
27 + 32 + 64, 27 + 32 + 98, 27 + 32 + 100, 27 + 32 + 102, 27 + 32 + 103, 27 + 32 + 107, 27 + 34 + 53,
27 + 34 + 54, 27 + 34 + 55, 27 + 34 + 57, 27 + 34 + 58, 27 + 34 + 64, 27 + 34 + 98, 27 + 34 + 100, 27 + 34 + 102,
27 + 34 + 103, 27 + 34 + 107, 27 + 53 + 54, 27 + 53 + 55, 27 + 53 + 57, 27 + 53 + 58, 27 + 53 + 64, 27 + 53 + 98,
27 + 53 + 100, 27 + 53 + 102, 27 + 53 + 103, 27 + 53 + 107, 27 + 54 + 55, 27 + 54 + 57, 27 + 54 + 58,
27 + 54 + 64, 27 + 54 + 98, 27 + 54 + 100, 27 + 54 + 102, 27 + 54 + 103, 27 + 54 + 107, 27 + 55 + 57,
27 + 55 + 58, 27 + 55 + 64, 27 + 55 + 98, 27 + 55 + 100, 27 + 55 + 102, 27 + 55 + 103, 27 + 55 + 107,
27 + 57 + 58, 27 + 57 + 64, 27 + 57 + 98, 27 + 57 + 100, 27 + 57 + 102, 27 + 57 + 103, 27 + 57 + 107,
27 + 58 + 64, 27 + 58 + 98, 27 + 58 + 100, 27 + 58 + 102, 27 + 58 + 103, 27 + 58 + 107, 27 + 64 + 98,
27 + 64 + 100, 27 + 64 + 102, 27 + 64 + 103, 27 + 64 + 107, 27 + 98 + 100, 27 + 98 + 102, 27 + 98 + 103,
27 + 98 + 107, 27 + 100 + 102, 27 + 100 + 103, 27 + 100 + 107, 27 + 102 + 103, 27 + 102 + 107,
27 + 103 + 107, 28 + 29 + 30, 28 + 29 + 31, 28 + 29 + 32, 28 + 29 + 34, 28 + 29 + 53, 28 + 29 + 54, 28 + 29 + 55,
28 + 29 + 57, 28 + 29 + 58, 28 + 29 + 64, 28 + 29 + 98, 28 + 29 + 100, 28 + 29 + 102, 28 + 29 + 103,
28 + 29 + 107, 28 + 30 + 31, 28 + 30 + 32, 28 + 30 + 34, 28 + 30 + 53, 28 + 30 + 54, 28 + 30 + 55, 28 + 30 + 57,
28 + 30 + 58, 28 + 30 + 64, 28 + 30 + 98, 28 + 30 + 100, 28 + 30 + 102, 28 + 30 + 103, 28 + 30 + 107,
28 + 31 + 32, 28 + 31 + 34, 28 + 31 + 53, 28 + 31 + 54, 28 + 31 + 55, 28 + 31 + 57, 28 + 31 + 58, 28 + 31 + 64,
28 + 31 + 98, 28 + 31 + 100, 28 + 31 + 102, 28 + 31 + 103, 28 + 31 + 107, 28 + 32 + 34, 28 + 32 + 53,
28 + 32 + 54, 28 + 32 + 55, 28 + 32 + 57, 28 + 32 + 58, 28 + 32 + 64, 28 + 32 + 98, 28 + 32 + 100, 28 + 32 + 102,
28 + 32 + 103, 28 + 32 + 107, 28 + 34 + 53, 28 + 34 + 54, 28 + 34 + 55, 28 + 34 + 57, 28 + 34 + 58, 28 + 34 + 64,
28 + 34 + 98, 28 + 34 + 100, 28 + 34 + 102, 28 + 34 + 103, 28 + 34 + 107, 28 + 53 + 54, 28 + 53 + 55,
28 + 53 + 57, 28 + 53 + 58, 28 + 53 + 64, 28 + 53 + 98, 28 + 53 + 100, 28 + 53 + 102, 28 + 53 + 103,
28 + 53 + 107, 28 + 54 + 55, 28 + 54 + 57, 28 + 54 + 58, 28 + 54 + 64, 28 + 54 + 98, 28 + 54 + 100,
28 + 54 + 102, 28 + 54 + 103, 28 + 54 + 107, 28 + 55 + 57, 28 + 55 + 58, 28 + 55 + 64, 28 + 55 + 98,
28 + 55 + 100, 28 + 55 + 102, 28 + 55 + 103, 28 + 55 + 107, 28 + 57 + 58, 28 + 57 + 64, 28 + 57 + 98,
28 + 57 + 100, 28 + 57 + 102, 28 + 57 + 103, 28 + 57 + 107, 28 + 58 + 64, 28 + 58 + 98, 28 + 58 + 100,
28 + 58 + 102, 28 + 58 + 103, 28 + 58 + 107, 28 + 64 + 98, 28 + 64 + 100, 28 + 64 + 102, 28 + 64 + 103,
28 + 64 + 107, 28 + 98 + 100, 28 + 98 + 102, 28 + 98 + 103, 28 + 98 + 107, 28 + 100 + 102, 28 + 100 + 103,
28 + 100 + 107, 28 + 102 + 103, 28 + 102 + 107, 28 + 103 + 107, 29 + 30 + 31, 29 + 30 + 32, 29 + 30 + 34,
29 + 30 + 53, 29 + 30 + 54, 29 + 30 + 55, 29 + 30 + 57, 29 + 30 + 58, 29 + 30 + 64, 29 + 30 + 98, 29 + 30 + 100,
29 + 30 + 102, 29 + 30 + 103, 29 + 30 + 107, 29 + 31 + 32, 29 + 31 + 34, 29 + 31 + 53, 29 + 31 + 54,
29 + 31 + 55, 29 + 31 + 57, 29 + 31 + 58, 29 + 31 + 64, 29 + 31 + 98, 29 + 31 + 100, 29 + 31 + 102,
29 + 31 + 103, 29 + 31 + 107, 29 + 32 + 34, 29 + 32 + 53, 29 + 32 + 54, 29 + 32 + 55, 29 + 32 + 57, 29 + 32 + 58,
29 + 32 + 64, 29 + 32 + 98, 29 + 32 + 100, 29 + 32 + 102, 29 + 32 + 103, 29 + 32 + 107, 29 + 34 + 53,
29 + 34 + 54, 29 + 34 + 55, 29 + 34 + 57, 29 + 34 + 58, 29 + 34 + 64, 29 + 34 + 98, 29 + 34 + 100, 29 + 34 + 102,
29 + 34 + 103, 29 + 34 + 107, 29 + 53 + 54, 29 + 53 + 55, 29 + 53 + 57, 29 + 53 + 58, 29 + 53 + 64, 29 + 53 + 98,
29 + 53 + 100, 29 + 53 + 102, 29 + 53 + 103, 29 + 53 + 107, 29 + 54 + 55, 29 + 54 + 57, 29 + 54 + 58,
29 + 54 + 64, 29 + 54 + 98, 29 + 54 + 100, 29 + 54 + 102, 29 + 54 + 103, 29 + 54 + 107, 29 + 55 + 57,
29 + 55 + 58, 29 + 55 + 64, 29 + 55 + 98, 29 + 55 + 100, 29 + 55 + 102, 29 + 55 + 103, 29 + 55 + 107,
29 + 57 + 58, 29 + 57 + 64, 29 + 57 + 98, 29 + 57 + 100, 29 + 57 + 102, 29 + 57 + 103, 29 + 57 + 107,
29 + 58 + 64, 29 + 58 + 98, 29 + 58 + 100, 29 + 58 + 102, 29 + 58 + 103, 29 + 58 + 107, 29 + 64 + 98,
29 + 64 + 100, 29 + 64 + 102, 29 + 64 + 103, 29 + 64 + 107, 29 + 98 + 100, 29 + 98 + 102, 29 + 98 + 103,
29 + 98 + 107, 29 + 100 + 102, 29 + 100 + 103, 29 + 100 + 107, 29 + 102 + 103, 29 + 102 + 107,
29 + 103 + 107, 30 + 31 + 32, 30 + 31 + 34, 30 + 31 + 53, 30 + 31 + 54, 30 + 31 + 55, 30 + 31 + 57, 30 + 31 + 58,
30 + 31 + 64, 30 + 31 + 98, 30 + 31 + 100, 30 + 31 + 102, 30 + 31 + 103, 30 + 31 + 107, 30 + 32 + 34,
30 + 32 + 53, 30 + 32 + 54, 30 + 32 + 55, 30 + 32 + 57, 30 + 32 + 58, 30 + 32 + 64, 30 + 32 + 98, 30 + 32 + 100,
30 + 32 + 102, 30 + 32 + 103, 30 + 32 + 107, 30 + 34 + 53, 30 + 34 + 54, 30 + 34 + 55, 30 + 34 + 57,
30 + 34 + 58, 30 + 34 + 64, 30 + 34 + 98, 30 + 34 + 100, 30 + 34 + 102, 30 + 34 + 103, 30 + 34 + 107,
30 + 53 + 54, 30 + 53 + 55, 30 + 53 + 57, 30 + 53 + 58, 30 + 53 + 64, 30 + 53 + 98, 30 + 53 + 100, 30 + 53 + 102,
30 + 53 + 103, 30 + 53 + 107, 30 + 54 + 55, 30 + 54 + 57, 30 + 54 + 58, 30 + 54 + 64, 30 + 54 + 98,
30 + 54 + 100, 30 + 54 + 102, 30 + 54 + 103, 30 + 54 + 107, 30 + 55 + 57, 30 + 55 + 58, 30 + 55 + 64,
30 + 55 + 98, 30 + 55 + 100, 30 + 55 + 102, 30 + 55 + 103, 30 + 55 + 107, 30 + 57 + 58, 30 + 57 + 64,
30 + 57 + 98, 30 + 57 + 100, 30 + 57 + 102, 30 + 57 + 103, 30 + 57 + 107, 30 + 58 + 64, 30 + 58 + 98,
30 + 58 + 100, 30 + 58 + 102, 30 + 58 + 103, 30 + 58 + 107, 30 + 64 + 98, 30 + 64 + 100, 30 + 64 + 102,
30 + 64 + 103, 30 + 64 + 107, 30 + 98 + 100, 30 + 98 + 102, 30 + 98 + 103, 30 + 98 + 107, 30 + 100 + 102,
30 + 100 + 103, 30 + 100 + 107, 30 + 102 + 103, 30 + 102 + 107, 30 + 103 + 107, 31 + 32 + 34, 31 + 32 + 53,
31 + 32 + 54, 31 + 32 + 55, 31 + 32 + 57, 31 + 32 + 58, 31 + 32 + 64, 31 + 32 + 98, 31 + 32 + 100, 31 + 32 + 102,
31 + 32 + 103, 31 + 32 + 107, 31 + 34 + 53, 31 + 34 + 54, 31 + 34 + 55, 31 + 34 + 57, 31 + 34 + 58, 31 + 34 + 64,
31 + 34 + 98, 31 + 34 + 100, 31 + 34 + 102, 31 + 34 + 103, 31 + 34 + 107, 31 + 53 + 54, 31 + 53 + 55,
31 + 53 + 57, 31 + 53 + 58, 31 + 53 + 64, 31 + 53 + 98, 31 + 53 + 100, 31 + 53 + 102, 31 + 53 + 103,
31 + 53 + 107, 31 + 54 + 55, 31 + 54 + 57, 31 + 54 + 58, 31 + 54 + 64, 31 + 54 + 98, 31 + 54 + 100,
31 + 54 + 102, 31 + 54 + 103, 31 + 54 + 107, 31 + 55 + 57, 31 + 55 + 58, 31 + 55 + 64, 31 + 55 + 98,
31 + 55 + 100, 31 + 55 + 102, 31 + 55 + 103, 31 + 55 + 107, 31 + 57 + 58, 31 + 57 + 64, 31 + 57 + 98,
31 + 57 + 100, 31 + 57 + 102, 31 + 57 + 103, 31 + 57 + 107, 31 + 58 + 64, 31 + 58 + 98, 31 + 58 + 100,
31 + 58 + 102, 31 + 58 + 103, 31 + 58 + 107, 31 + 64 + 98, 31 + 64 + 100, 31 + 64 + 102, 31 + 64 + 103,
31 + 64 + 107, 31 + 98 + 100, 31 + 98 + 102, 31 + 98 + 103, 31 + 98 + 107, 31 + 100 + 102, 31 + 100 + 103,
31 + 100 + 107, 31 + 102 + 103, 31 + 102 + 107, 31 + 103 + 107, 32 + 34 + 53, 32 + 34 + 54, 32 + 34 + 55,
32 + 34 + 57, 32 + 34 + 58, 32 + 34 + 64, 32 + 34 + 98, 32 + 34 + 100, 32 + 34 + 102, 32 + 34 + 103,
32 + 34 + 107, 32 + 53 + 54, 32 + 53 + 55, 32 + 53 + 57, 32 + 53 + 58, 32 + 53 + 64, 32 + 53 + 98, 32 + 53 + 100,
32 + 53 + 102, 32 + 53 + 103, 32 + 53 + 107, 32 + 54 + 55, 32 + 54 + 57, 32 + 54 + 58, 32 + 54 + 64,
32 + 54 + 98, 32 + 54 + 100, 32 + 54 + 102, 32 + 54 + 103, 32 + 54 + 107, 32 + 55 + 57, 32 + 55 + 58,
32 + 55 + 64, 32 + 55 + 98, 32 + 55 + 100, 32 + 55 + 102, 32 + 55 + 103, 32 + 55 + 107, 32 + 57 + 58,
32 + 57 + 64, 32 + 57 + 98, 32 + 57 + 100, 32 + 57 + 102, 32 + 57 + 103, 32 + 57 + 107, 32 + 58 + 64,
32 + 58 + 98, 32 + 58 + 100, 32 + 58 + 102, 32 + 58 + 103, 32 + 58 + 107, 32 + 64 + 98, 32 + 64 + 100,
32 + 64 + 102, 32 + 64 + 103, 32 + 64 + 107, 32 + 98 + 100, 32 + 98 + 102, 32 + 98 + 103, 32 + 98 + 107,
32 + 100 + 102, 32 + 100 + 103, 32 + 100 + 107, 32 + 102 + 103, 32 + 102 + 107, 32 + 103 + 107,
34 + 53 + 54, 34 + 53 + 55, 34 + 53 + 57, 34 + 53 + 58, 34 + 53 + 64, 34 + 53 + 98, 34 + 53 + 100, 34 + 53 + 102,
34 + 53 + 103, 34 + 53 + 107, 34 + 54 + 55, 34 + 54 + 57, 34 + 54 + 58, 34 + 54 + 64, 34 + 54 + 98,
34 + 54 + 100, 34 + 54 + 102, 34 + 54 + 103, 34 + 54 + 107, 34 + 55 + 57, 34 + 55 + 58, 34 + 55 + 64,
34 + 55 + 98, 34 + 55 + 100, 34 + 55 + 102, 34 + 55 + 103, 34 + 55 + 107, 34 + 57 + 58, 34 + 57 + 64,
34 + 57 + 98, 34 + 57 + 100, 34 + 57 + 102, 34 + 57 + 103, 34 + 57 + 107, 34 + 58 + 64, 34 + 58 + 98,
34 + 58 + 100, 34 + 58 + 102, 34 + 58 + 103, 34 + 58 + 107, 34 + 64 + 98, 34 + 64 + 100, 34 + 64 + 102,
34 + 64 + 103, 34 + 64 + 107, 34 + 98 + 100, 34 + 98 + 102, 34 + 98 + 103, 34 + 98 + 107, 34 + 100 + 102,
34 + 100 + 103, 34 + 100 + 107, 34 + 102 + 103, 34 + 102 + 107, 34 + 103 + 107, 53 + 54 + 55, 53 + 54 + 57,
53 + 54 + 58, 53 + 54 + 64, 53 + 54-98, 53 + 54 + 100, 53 + 54 + 102, 53 + 54 + 103, 53 + 54 + 107,
53 + 55 + 57, 53 + 55 + 58, 53 + 55 + 64, 53 + 55 + 98, 53 + 55 + 100, 53 + 55 + 102, 53 + 55 + 103,
53 + 55 + 107, 53 + 57 + 58, 53 + 57 + 64, 53 + 57 + 98, 53 + 57 + 100, 53 + 57 + 102, 53 + 57 + 103,
53 + 57 + 107, 53 + 58 + 64, 53 + 58 + 98, 53 + 58 + 100, 53 + 58 + 102, 53 + 58 + 103, 53 + 58 + 107,
53 + 64 + 98, 53 + 64 + 100, 53 + 64 + 102, 53 + 64 + 103, 53 + 64 + 107, 53 + 98 + 100, 53 + 98 + 102,
53 + 98 + 103, 53 + 98 + 107, 53 + 100 + 102, 53 + 100 + 103, 53 + 100 + 107, 53 + 102 + 103,
53 + 102 + 107, 53 + 103 + 107, 54 + 55 + 57, 54 + 55 + 58, 54 + 55 + 64, 54 + 55 + 98, 54 + 55 + 100,
54 + 55 + 102, 54 + 55 + 103, 54 + 55 + 107, 54 + 57 + 58, 54 + 57 + 64, 54 + 57 + 98, 54 + 57 + 100,
54 + 57 + 102, 54 + 57 + 103, 54 + 57 + 107, 54 + 58 + 64, 54 + 58 + 98, 54 + 58 + 100, 54 + 58 + 102,
54 + 58 + 103, 54 + 58 + 107, 54 + 64 + 98, 54 + 64 + 100, 54 + 64 + 102, 54 + 64 + 103, 54 + 64 + 107,
54 + 98 + 100, 54 + 98 + 102, 54 + 98 + 103, 54 + 98 + 107, 54 + 100 + 102, 54 + 100 + 103, 54 + 100 + 107,
54 + 102 + 103, 54 + 102 + 107, 54 + 103 + 107, 55 + 57 + 58, 55 + 57 + 64, 55 + 57 + 98, 55 + 57 + 100,
55 + 57 + 102, 55 + 57 + 103, 55 + 57 + 107, 55 + 58 + 64, 55 + 58 + 98, 55 + 58 + 100, 55 + 58 + 102,
55 + 58 + 103, 55 + 58 + 107, 55 + 64 + 98, 55 + 64 + 100, 55 + 64 + 102, 55 + 64 + 103, 55 + 64 + 107,
55 + 98 + 100, 55 + 98 + 102, 55 + 98 + 103, 55 + 98 + 107, 55 + 100 + 102, 55 + 100 + 103, 55 + 100 + 107,
55 + 102 + 103, 55 + 102 + 107, 55 + 103 + 107, 57 + 58 + 64, 57 + 58 + 98, 57 + 58 + 100, 57 + 58 + 102,
57 + 58 + 103, 57 + 58 + 107, 57 + 64 + 98, 57 + 64 + 100, 57 + 64 + 102, 57 + 64 + 103, 57 + 64 + 107,
57 + 98 + 100, 57 + 98 + 102, 57 + 98 + 103, 57 + 98 + 107, 57 + 100 + 102, 57 + 100 + 103, 57 + 100 + 107,
57 + 102 + 103, 57 + 102 + 107, 57 + 103 + 107, 58 + 64 + 98, 58 + 64 + 100, 58 + 64 + 102, 58 + 64 + 103,
58 + 64 + 107, 58 + 98 + 100, 58 + 98 + 102, 58 + 98 + 103, 58 + 98 + 107, 58 + 100 + 102, 58 + 100 + 103,
58 + 100 + 107, 58 + 102 + 103, 58 + 102 + 107, 58 + 103 + 107, 64 + 98 + 100, 64 + 98 + 102, 64 + 98 + 103,
64 + 98 + 107, 64 + 100 + 102, 64 + 100 + 103, 64 + 100 + 107, 64 + 102 + 103, 64 + 102 + 107,
64 + 103 + 107, 98 + 100 + 102, 98 + 100 + 103, 98 + 100 + 107, 98 + 102 + 103, 98 + 102 + 107,
98 + 103 + 107, 100 + 102 + 103, 100 + 102 + 107, 100 + 103 + 107, or 102 + 103 + 107

In some examples of any of the ABPCs described herein, an LCVD includes an LCVD that is at least 90% (e.g., at least 92%, at least 94%, at least 96%, at least 98%, at least 99%, or 100%) identical to SEQ ID NO: 2, where the LCVD includes a histidine at any of the specific combinations of one or more amino acid positions in SEQ ID NO: 2 listed in Table 2.

In some examples of any of the ABPCs described herein, the first ABD includes an LCVD that is at least 90% (e.g., at least 92%, at least 94%, at least 96%, at least 98%, at least 99%, or 100%) identical to SEQ ID NO: 2, where the LCVD includes an alanine at position 38 in SEQ ID NO: 2.

In some examples of any of the ABPCs described herein the first ABD includes: an LCVD that is at least 90% (e.g., at least 92%, at least 94%, at least 96%, at least 98%, at least 99%, or 100%) identical to SEQ ID NO: 2, where the LCVD includes an alanine at position 38 in SEQ ID NO: 2, and an HCVD that is at least 90% (e.g., at least 92%, at least 94%, at least 96%, at least 98%, at least 99%, or 100%) identical to SEQ ID NO: 1, where the HCVD includes a histidine at one or more amino acid positions in SEQ ID NO: 1 selected from the group consisting of: 24, 27, 28, 29, 30, 31, 32, 34, 53, 54, 55, 57, 58, 64, 98, 100, 102, 103, or 107.

In some examples of any of the ABPCs described herein, the first ABD includes an LCVD that is at least 90% (e.g., at least 92%, at least 94%, at least 96%, at least 98%, at least 99%, or 100%) identical to SEQ ID NO: 2, where the LCVD includes a histidine at one or more amino acid positions in SEQ ID NO: 2 selected from the group consisting of: 25, 29, 31, 35, 60, 93, or 94 and where the LCVD includes an alanine at position 38 in SEQ ID NO: 2. In some examples of any of the ABPCs described herein the first ABD includes: an LCVD that is at least 90% (e.g., at least 92%, at least 94%, at least 96%, at least 98%, at least 99%, or 100%) identical to SEQ ID NO: 2, where the LCVD includes a histidine at one or more amino acid positions in SEQ ID NO: 2 selected from the group consisting of: 25, 29, 31, 35, 60, 93, or 94 and where the LCVD includes an alanine at position 38 in SEQ ID NO: 2, and an HCVD that is at least 90% (e.g., at least 92%, at least 94%, at least 96%, at least 98%, at least 99%, or 100%) identical to SEQ ID NO: 1, where the HCVD includes a histidine at one or more amino acid positions in SEQ ID NO: 1 selected from the group consisting of: 24, 27, 28, 29, 30, 31, 32, 34, 53, 54, 55, 57, 58, 64, 98, 100, 102, 103, or 107.

In some examples of any of the ABPCs described herein the first ABD includes: an LCVD that is at least 90% (e.g., at least 92%, at least 94%, at least 96%, at least 98%, at least 99%, or 100%) identical to SEQ ID NO: 2, where the LCVD includes a histidine at one or more amino acid positions in SEQ ID NO: 2 selected from the group consisting of: 25, 29, 31, 35, 60, 93, or 94 and where the LCVD includes an alanine at position 38 in SEQ ID NO: 2, and an HCVD that is at least 90% (e.g., at least 92%, at least 94%, at least 96%, at least 98%, at least 99%, or 100%) identical to SEQ ID NO: 1, where the HCVD includes a histidine at one or more amino acid positions in SEQ ID NO: 1 selected from the group consisting of: 24, 27, 28, 29, 30, 31, 32, 34, 53, 54, 55, 57, 58, 64, 98, 100, 102, 103, or 107 and wherein the HCVD includes an alanine at position 35.

In some examples of any of the ABPCs described herein, an LCVD includes an LCVD that is at least 90% (e.g., at least 92%, at least 94%, at least 96%, at least 98%, at least 99%, or 100%) identical to SEQ ID NO: 2, where the LCVD includes a histidine at any of the specific combinations of one or more amino acid positions in SEQ ID NO: 2 listed in Table 2 and where the LCVD includes an alanine at position 38 of SEQ ID NO: 2.

TABLE 2
Exemplary Combinations of Amino Acid Positions in SEQ ID NO: 2 that can be Substituted with Histidine

25 + 29, 25 + 31, 25 + 35, 25 + 60, 25 + 93, 25 + 94, 29 + 31, 29 + 35, 29 + 60, 29 + 93, 29 + 94, 31 + 35,
31 + 60, 31 + 93, 31 + 94, 35 + 60, 35 + 93, 35 + 94, 60 + 93, 60 + 94, 93 + 94, 25 + 29 + 31, 25 + 29 + 35,
25 + 29 + 60, 25 + 29 + 93, 25 + 29 + 94, 25 + 31 + 35, 25 + 31 + 60, 25 + 31 + 93, 25 + 31 + 94, 25 + 35 + 60,
25 + 35 + 93, 25 + 35 + 94, 25 + 60 + 93, 25 + 60 + 94, 25 + 93 + 94, 29 + 31 + 35, 29 + 31 + 60, 29 + 31 + 93,
29 + 31 + 94, 29 + 35 + 60, 29 + 35 + 93, 29 + 35 + 94, 29 + 60 + 93, 29 + 60 + 94, 29 + 93 + 94, 31 + 35 + 60,
31 + 35 + 93, 31 + 35 + 94, 31 + 60 + 93, 31 + 60 + 94, 31 + 93 + 94, 35 + 60 + 93, 35 + 60 + 94, 35 + 93 + 94, or
60 + 93 + 94

In some examples of any of the ABPCs described herein, the first ABD includes an HCVD that is at least 90% (e.g., at least 92%, at least 94%, at least 96%, at least 98%, at least 99%, or 100%) identical to SEQ ID NO: 1, where the HCVD includes a histidine at any of the specific combinations of one or more amino acid positions in SEQ ID NO: 1 listed in Table 1; and an LCVD that that is at least 90% (e.g., at least 92%, at least 94%, at least 96%, at least 98%, at least 99%, or 100%) identical to SEQ ID NO: 2, where the LCVD includes a histidine at any of the specific combinations of one or more amino acid positions in SEQ ID NO: 2 listed in Table 2.

In some examples of any of the ABPCs described herein, the first ABD includes an LCVD that is at least 90% (e.g., at least 92%, at least 94%, at least 96%, at least 98%, at least 99%, or 100%) identical to SEQ ID NO: 2, where the LCVD includes a histidine at any of the specific combinations of one or more amino acid positions in SEQ ID NO: 2 listed in Table 2, and where the LCVD includes an alanine at position 38 in SEQ ID NO: 2; and an HCVD that that is at least 90% (e.g., at least 92%, at least 94%, at least 96%, at least 98%, at least 99%, or 100%) identical to SEQ ID NO: 1, where the HCVD includes a histidine at any of the specific combinations of one or more amino acid positions in SEQ ID NO: 1 listed in Table 1.

In some examples of any of the ABPCs described herein, the first ABD comprises an LCVD comprising SEQ ID NO: 2, and an HCVD that is at least 90% identical (e.g., at least 92%, at least 94%, at least 96%, at least 98%, at least 99%, or 100%) identical to SEQ ID NO: 1, where the HCVD includes a histidine at any of the specific combinations of one or more (e.g., two, three, four, five, six, seven, eight, nine, or ten) amino acid positions in SEQ ID NO: 1 listed in Table 1.

In some examples of any of the ABPCs described herein, the first ABD comprises an LCVD that is at least 90% identical to SEQ ID NO: 2, wherein the LCVD includes a histidine at position 25 in SEQ ID NO: 2; and an HCVD that is at least 90% (e.g., at least 92%, at least 94%, at least 96%, at least 98%, at least 99%, or 100%) identical to SEQ ID NO: 1, where the HCVD includes a histidine at any of the specific combinations of one or more amino acid positions in SEQ ID NO: 1 listed in Table 1.

In some examples of any of the ABPCs described herein, the first ABD comprises an LCVD that is at least 90% identical to SEQ ID NO: 2, wherein the LCVD includes a histidine at position 29 in SEQ ID NO: 2; and an HCVD that is at least 90% (e.g., at least 92%, at least 94%, at least 96%, at least 98%, at least 99%, or 100%) identical to SEQ ID NO: 1, where the HCVD includes a histidine at any of the specific combinations of one or more amino acid positions in SEQ ID NO: 1 listed in Table 1.

In some examples of any of the ABPCs described herein, the first ABD comprises an LCVD that is at least 90% identical to SEQ ID NO: 2, wherein the LCVD includes a histidine at position 31 in SEQ ID NO: 2; and an HCVD that is at least 90% (e.g., at least 92%, at least 94%, at least 96%, at least 98%, at least 99%, or 100%) identical to SEQ ID NO: 1, where the HCVD includes a histidine at any of the specific combinations of one or more amino acid positions in SEQ ID NO: 1 listed in Table 1.

In some examples of any of the ABPCs described herein, the first ABD comprises an LCVD that is at least 90% identical to SEQ ID NO: 2, wherein the LCVD includes a histidine at position 35 in SEQ ID NO: 2; and an HCVD that is at least 90% (e.g., at least 92%, at least 94%, at least 96%, at least 98%, at least 99%, or 100%) identical to SEQ ID NO: 1, where the HCVD includes a histidine at any of the specific combinations of one or more amino acid positions in SEQ ID NO: 1 listed in Table 1.

In some examples of any of the ABPCs described herein, the first ABD comprises an LCVD that is at least 90% identical to SEQ ID NO: 2, wherein the LCVD includes a histidine at position 60 in SEQ ID NO: 2; and an HCVD that is at least 90% (e.g., at least 92%, at least 94%, at least 96%, at least 98%, at least 99%, or 100%) identical to SEQ ID NO: 1, where the HCVD includes a histidine at any of the specific combinations of one or more amino acid positions in SEQ ID NO: 1 listed in Table 1.

In some examples of any of the ABPCs described herein, the first ABD comprises an LCVD that is at least 90% identical to SEQ ID NO: 2, wherein the LCVD includes a histidine at position 93 in SEQ ID NO: 2; and an HCVD that is at least 90% (e.g., at least 92%, at least 94%, at least 96%, at least 98%, at least 99%, or 100%) identical to SEQ ID NO: 1, where the HCVD includes a histidine at any of the specific combinations of one or more amino acid positions in SEQ ID NO: 1 listed in Table 1.

In some examples of any of the ABPCs described herein, the first ABD comprises an LCVD that is at least 90% identical to SEQ ID NO: 2, wherein the LCVD includes a histidine at position 94 in SEQ ID NO: 2; and an HCVD that is at least 90% (e.g., at least 92%, at least 94%, at least 96%, at least 98%, at least 99%, or 100%) identical to SEQ ID NO: 1, where the HCVD includes a histidine at any of the specific combinations of one or more amino acid positions in SEQ ID NO: 1 listed in Table 1.

In some examples of any of the ABPCs described herein, the first ABD includes an HCVD of cofetuzumab with one or more histidine(s) substituted with an alanine. In some examples of any of the ABPCs described herein, the first ABD includes an LCVD of cofetuzumab with one or more histidine(s) substituted with an alanine. In some examples of any of the ABPCs described herein, the first ABD includes an HCVD of cofetuzumab with one or more histidines substituted with an alanine; and an LCVD of cofetuzumab with one or more histidine(s) substituted with an alanine. In some examples of any of the ABPCs described herein, the HCVD of cofetuzumab comprises SEQ ID NO: 1. In some examples of any of the ABPCs described herein, the LCVD of cofetuzumab comprises SEQ ID NO: 2.

In some examples of any of the ABPCs described herein, the first ABD comprises an HCVD comprising a CDR1, a CDR2, and a CDR3 of SEQ ID NO: 3, SEQ ID NO: 4, and SEQ ID NO: 5, respectively, with collectively a total of one or more (e.g., one, two, three, four, five, six, seven, eight, nine, or ten) histidine(s) in SEQ ID NOs: 3, 4, and 5 substituted with an alanine. In some examples of any of the ABPCs described herein, the first ABD comprises an LCVD comprising a CDR1, a CDR2, and a CDR3 of SEQ ID NO: 6, SEQ ID NO: 7, and SEQ ID NO: 8, respectively, with collectively a total of one or more (e.g., one, two, three, four, five, six, seven, eight, nine, or ten) histidine(s) in SEQ ID NOs: 6, 7, and 8 substituted with an alanine. In some examples of any of the ABPCs described herein, the first ABD includes: an HCVD comprising a CDR1, a CDR2, and a CDR3 of SEQ ID NO: 3, SEQ ID NO: 4, and SEQ ID NO: 5, respectively, with collectively a total of one or more (e.g., one, two, three, four, five, six, seven, eight, nine, or ten) histidine(s) in SEQ ID NOs: 3, 4, and 5 substituted with an alanine; and an LCVD comprising a CDR1, a CDR2, and a CDR3 of SEQ ID NO: 6, SEQ ID NO: 7, and SEQ ID NO: 8, respectively, with collectively a total of one or more (e.g., one, two, three, four, five, six, seven, eight, nine, or ten) histidine(s) in SEQ ID NOs: 6, 7, and 8 substituted with an alanine.

In some examples of any of the ABPCs described herein, the first ABD comprises an HCVD of SEQ ID NO: 1, or one of 13-54, or one of 86-105. In some examples of any of the ABPCs described herein, the first ABD includes an LCVD of SEQ ID NO: 2, or one 55-85, or one of 106-125.

In some examples of any of the ABPCs described herein, the first ABD includes an LCVD comprising SEQ ID NO: 2, and an HCVD comprising: SEQ ID NO: 1, or one of 13-54, or one of 86-105. In some examples of any of the ABPCs described herein, the first ABD includes an LCVD comprising SEQ ID NO: 55, and an HCVD comprising: SEQ ID NO: 1, or one of 13-54, or one of 86-105. In some examples of any of the ABPCs described herein, the first ABD includes an LCVD comprising SEQ ID NO: 56, and an HCVD comprising: SEQ ID NO: 1, or one of 13-54, or one of 86-105. In some examples of any of the ABPCs described herein, the first ABD includes an LCVD comprising SEQ ID NO: 57, and an HCVD comprising: SEQ ID NO: 1, or one of 13-54, or one of 86-105. In some examples of any of the ABPCs described herein, the first ABD includes an LCVD comprising SEQ ID NO: 58, and an HCVD comprising: SEQ ID NO: 1, or one of 13-54, or one of 86-105. In some examples of any of the ABPCs described herein, the first ABD includes an LCVD comprising SEQ ID NO: 59, and an HCVD comprising: SEQ ID NO: 1, or one of 13-54, or one of 86-105. In some examples of any of the ABPCs described herein, the first ABD includes an LCVD comprising SEQ ID NO: 60, and an HCVD comprising: SEQ ID NO: 1, or one of 13-54, or one of 86-105. In some examples of any of the ABPCs described herein, the first ABD includes an LCVD comprising SEQ ID NO: 61, and an HCVD comprising: SEQ ID NO: 1, or one of 13-54, or one of 86-105. In some examples of any of the ABPCs described herein, the first ABD includes an LCVD comprising SEQ ID NO: 62, and an HCVD comprising: SEQ ID NO: 1, or one of 13-54, or one of 86-105. In some examples of any of the ABPCs described herein, the first ABD includes an LCVD comprising SEQ ID NO: 63, and an HCVD comprising: SEQ ID NO: 1, or one of 13-54, or one of 86-105. In some examples of any of the ABPCs described herein, the first ABD includes an LCVD comprising SEQ ID NO: 64, and an HCVD comprising: SEQ ID NO: 1, or one of 13-54, or one of 86-105. In some examples of any of the ABPCs described herein, the first ABD includes an LCVD comprising SEQ ID NO: 65, and an HCVD comprising: SEQ ID NO: 1, or one of 13-54, or one of 86-105. In some examples of any of the ABPCs described herein, the first ABD includes an LCVD comprising SEQ ID NO: 66, and an HCVD comprising: SEQ ID NO: 1, or one of 13-54, or one of 86-105. In some examples of any of the ABPCs described herein, the first ABD includes an LCVD comprising SEQ ID NO: 67, and an HCVD comprising: SEQ ID NO: 1, or one of 13-54, or one of 86-105. In some examples of any of the ABPCs described herein, the first ABD includes an LCVD comprising SEQ ID NO: 68, and an HCVD comprising: SEQ ID NO: 1, or one of 13-54, or one of 86-105. In some examples of any of the ABPCs described herein, the first ABD includes an LCVD comprising SEQ ID NO: 69, and an HCVD comprising: SEQ ID NO: 1, or one of 13-54, or one of 86-105. In some examples of any of the ABPCs described herein, the first ABD includes an LCVD comprising SEQ ID NO: 70, and an HCVD comprising: SEQ ID NO: 1, or one of 13-54, or one of 86-105. In some examples of any of the ABPCs described herein, the first ABD includes an LCVD comprising SEQ ID NO: 71, and an HCVD comprising: SEQ ID NO: 1, or one of 13-54, or one of 86-105. In some examples of any of the ABPCs described herein, the first ABD includes an LCVD comprising SEQ ID NO: 72 and an HCVD comprising: SEQ ID NO: 1, or one of 13-54, or one of 86-105. In some examples of any of the ABPCs described herein, the first ABD includes an LCVD comprising SEQ ID NO: 73, and an HCVD comprising: SEQ ID NO: 1, or one of 13-54, or one of 86-105. In some examples of any of the ABPCs described herein, the first ABD includes an LCVD comprising SEQ ID NO: 74, and an HCVD comprising: SEQ ID NO: 1, or one of 13-54, or one of 86-105. In some examples of any of the ABPCs described herein, the first ABD includes an LCVD comprising SEQ ID NO: 75, and an HCVD comprising: SEQ ID NO: 1, or one of 13-54, or one of 86-105. In some examples of any of the ABPCs described herein, the first ABD includes an LCVD comprising SEQ ID NO: 76, and an HCVD comprising: SEQ ID NO: 1, or one of 13-54, or one of 86-105. In some examples of any of the ABPCs described herein, the first ABD includes an LCVD comprising SEQ ID NO: 77, and an HCVD comprising: SEQ ID NO: 1, or one of 13-54, or one of 86-105. In some examples of any of the ABPCs described herein, the first ABD includes an LCVD comprising SEQ ID NO: 78, and an HCVD comprising: SEQ ID NO: 1, or one of 13-54, or one of 86-105. In some examples of any of the ABPCs described herein, the first ABD includes an LCVD comprising SEQ ID NO: 79, and an HCVD comprising: SEQ ID NO: 1, or one of 13-54, or one of 86-105. In some examples of any of the ABPCs described herein, the first ABD includes an LCVD comprising SEQ ID NO: 80, and an HCVD comprising: SEQ ID NO: 1, or one of 13-54, or one of 86-105. In some examples of any of the ABPCs described herein, the first ABD includes an LCVD comprising SEQ ID NO: 81, and an HCVD comprising: SEQ ID NO: 1, or one of 13-54, or one of 86-105. In some examples of any of the ABPCs described herein, the first ABD includes an LCVD comprising SEQ ID NO: 82, and an HCVD comprising: SEQ ID NO: 1, or one of 13-54, or one of 86-105. In some examples of any of the ABPCs described herein, the first ABD includes an LCVD comprising SEQ ID NO: 83, and an HCVD comprising: SEQ ID NO: 1, or one of 13-54, or one of 86-105. In some examples of any of the ABPCs described herein, the first ABD includes an LCVD comprising SEQ ID NO: 84, and an HCVD comprising: SEQ ID NO: 1, or one of 13-54, or one of 86-105. In some examples of any of the ABPCs described herein, the first ABD includes an LCVD comprising SEQ ID NO: 85, and an HCVD comprising: SEQ ID NO: 1, or one of 13-54, or one of 86-105. In some examples of any of the ABPCs described herein, the first ABD includes an LCVD comprising SEQ ID NO: 106, and an HCVD comprising: SEQ ID NO: 1, or one of 13-54, or one of 86-105. In some examples of any of the ABPCs described herein, the first ABD includes an LCVD comprising SEQ ID NO: 107, and an HCVD comprising: SEQ ID NO: 1, or one of 13-54, or one of 86-105. In some examples of any of the ABPCs described herein, the first ABD includes an LCVD comprising SEQ ID NO: 108, and an HCVD comprising: SEQ ID NO: 1, or one of 13-54, or one of 86-105. In some examples of any of the ABPCs described herein, the first ABD includes an LCVD comprising SEQ ID NO: 109, and an HCVD comprising: SEQ ID NO: 1, or one of 13-54, or one of 86-105. In some examples of any of the ABPCs described herein, the first ABD includes an LCVD comprising SEQ ID NO: 110, and an HCVD comprising: SEQ ID NO: 1, or one of 13-54, or one of 86-105. In some examples of any of the ABPCs described herein, the first ABD includes an LCVD comprising SEQ ID NO: 111, and an HCVD comprising: SEQ ID NO: 1, or one of 13-54, or one of 86-105. In some examples of any of the ABPCs described herein, the first ABD includes an LCVD comprising SEQ ID NO: 112, and an HCVD comprising: SEQ ID NO: 1, or one of 13-54, or one of 86-105. In some examples of any of the ABPCs described herein, the first ABD includes an LCVD comprising SEQ ID NO: 113, and an HCVD comprising: SEQ ID NO: 1, or one of 13-54, or one of 86-105. In some examples of any of the ABPCs described herein, the first ABD includes an LCVD comprising SEQ ID NO: 114, and an HCVD comprising: SEQ ID NO: 1, or one of 13-54, or one of 86-105. In some examples of any of the ABPCs described herein, the first ABD includes an LCVD comprising SEQ ID NO: 115, and an HCVD comprising: SEQ ID NO: 1, or one of 13-54, or one of 86-105. In some examples of any of the ABPCs described herein, the first ABD includes an LCVD comprising SEQ ID NO: 116, and an HCVD comprising: SEQ ID NO: 1, or one of 13-54, or one of 86-105. In some examples of any of the ABPCs described herein, the first ABD includes an LCVD comprising SEQ ID NO: 117, and an HCVD comprising: SEQ ID NO: 1, or one of 13-54, or one of 86-105. In some examples of any of the ABPCs described herein, the first ABD includes an LCVD comprising SEQ ID NO: 118, and an HCVD comprising: SEQ ID NO: 1, or one of 13-54, or one of 86-105. In some examples of any of the ABPCs described herein, the first ABD includes an LCVD comprising SEQ ID NO: 119, and an HCVD comprising: SEQ ID NO: 1, or one of 13-54, or one of 86-105. In some examples of any of the ABPCs described herein, the first ABD includes an LCVD comprising SEQ ID NO: 120, and an HCVD comprising: SEQ ID NO: 1, or one of 13-54, or one of 86-105. In some examples of any of the ABPCs described herein, the first ABD includes an LCVD comprising SEQ ID NO: 121, and an HCVD comprising: SEQ ID NO: 1, or one of 13-54, or one of 86-105. In some examples of any of the ABPCs described herein, the first ABD includes an LCVD comprising SEQ ID NO: 122, and an HCVD comprising: SEQ ID NO: 1, or one of 13-54, or one of 86-105. In some examples of any of the ABPCs described herein, the first ABD includes an LCVD comprising SEQ ID NO: 123, and an HCVD comprising: SEQ ID NO: 1, or one of 13-54, or one of 86-105. In some examples of any of the ABPCs described herein, the first ABD includes an LCVD comprising SEQ ID NO: 124, and an HCVD comprising: SEQ ID NO: 1, or one of 13-54, or one of 86-105. In some examples of any of the ABPCs described herein, the first ABD includes an LCVD comprising SEQ ID NO: 125, and an HCVD comprising: SEQ ID NO: 1, or one of 13-54, or one of 86-105.

In some examples of any of the ABPCs described herein, the first ABD includes an HCVD of a 7C8 with one or more amino acids substituted with a histidine. In some examples of any of the ABPCs described herein, the first ABD includes an LCVD of 7C8 with one or more amino acids substituted with a histidine. In some examples of any of the ABPCs described herein, the first ABD includes an HCVD of 7C8 with one or more amino acids substituted with a histidine; and an LCVD of 7C8 with one or more amino acids substituted with a histidine. In some examples of any of the ABPCs described herein, the HCVD of 7C8 comprises SEQ ID NO: 126. In some examples of any of the ABPCs described herein, the LCVD of 7C8 comprises SEQ ID NO: 127.

In some examples of any of the ABPCs described herein, the first ABD comprises an HCVD comprising a CDR1, a CDR2, and a CDR3 of SEQ ID NO: 128, SEQ ID NO: 129, and SEQ ID NO: 130, respectively, with collectively a total of one or more (e.g., one, two, three, four, five, six, seven, eight, nine, or ten) amino acid positions in SEQ ID NOs: 128, 129, and 130 substituted with a histidine. In some examples of any of the ABPCs described herein, the first ABD comprises an LCVD comprising a CDR1, a CDR2, and a CDR3 of SEQ ID NO: 131, SEQ ID NO: 132, and SEQ ID NO: 133, respectively, with collectively a total of one or more (e.g., one, two, three, four, five, six, seven, eight, nine, or ten) amino acid positions in SEQ ID NOs: 131, 132, and 133 substituted with a histidine. In some examples of any of the ABPCs described herein, the first ABD includes: an HCVD comprising a CDR1, a CDR2, and a CDR3 of SEQ ID NO: 128, SEQ ID NO: 129, and SEQ ID NO: 130, respectively, with collectively a total of one or more (e.g., one, two, three, four, five, six, seven, eight, nine, or ten) amino acid positions in SEQ ID NOs: 128, 129, and 130 substituted with a histidine; and an LCVD comprising a CDR1, a CDR2, and a CDR3 of SEQ ID NO: 131, SEQ ID NO: 132, and SEQ ID NO: 133, respectively, with collectively a total of one or more (e.g., one, two, three, four, five, six, seven, eight, nine, or ten) amino acid positions in SEQ ID NOs: 131, 132, and 133 substituted with a histidine.

In some examples of any of the ABPCs described herein, the first ABD includes an HCVD that is at least 90% (e.g., at least 92%, at least 94%, at least 96%, at least 98%, at least 99%, or 100%) identical to SEQ ID NO: 126, where the HCVD includes a histidine at one or more amino acid positions in SEQ ID NO: 126 selected from the group consisting of: 53, 54, 56, 57, 60, 102, 103, 104, 105, 106, 108, 109, 110, and 111. In some examples of any of the ABPCs described herein, the first ABD includes an LCVD that is at least 90% (e.g., at least 92%, at least 94%, at least 96%, at least 98%, at least 99%, or 100%) identical to SEQ ID NO: 127, where the LCVD includes a histidine at one or more amino acid positions in SEQ ID NO: 127 selected from the group of: 25, 26, 28, 29, 31, 32, 33, 50, 90, 92, and 94. In some examples of any of the ABPCs described herein the first ABD includes: an HCVD that is at least 90% (e.g., at least 92%, at least 94%, at least 96%, at least 98%, at least 99%, or 100%) identical to SEQ ID NO: 126, where the HCVD includes a histidine at one or more amino acid positions in SEQ ID NO: 126 selected from the group consisting of: 53, 54, 56, 57, 60, 102, 103, 104, 105, 106, 108, 109, 110, and 111, and an LCVD that is at least 90% (e.g., at least 92%, at least 94%, at least 96%, at least 98%, at least 99%, or 100%) identical to SEQ ID NO: 127, where the LCVD includes a histidine at one or more amino acid positions in SEQ ID NO: 127 selected from the group consisting of: 25, 26, 28, 29, 31, 32, 33, 50, 90, 92, and 94.

In some examples of any of the ABPCs described herein, an HCVD includes an HCVD that is at least 90% (e.g., at least 92%, at least 94%, at least 96%, at least 98%, at least 99%, or 100%) identical to SEQ ID NO: 126, where the HCVD includes a histidine at any of the specific combinations of one or more amino acid positions in SEQ ID NO: 126 listed in Table 3.

TABLE 3
Exemplary Combinations of Amino Acid Positions in SEQ ID NO: 126 that can be Substituted with Histidine

53 + 54, 53 + 56, 53 + 57, 53 + 60, 53 + 102, 53 + 103, 53 + 104, 53 + 105, 53 + 106, 53 + 108, 53 + 109,
53 + 110, 53 + 111, 54 + 56, 54 + 57, 54 + 60, 54 + 102, 54 + 103, 54 + 104, 54 + 105, 54 + 106, 54 + 108,
54 + 109, 54 + 110, 54 + 111, 56 + 57, 56 + 60, 56 + 102, 56 + 103, 56 + 104, 56 + 105, 56 + 106, 56 + 108,
56 + 109, 56 + 110, 56 + 111, 57 + 60, 57 + 102, 57 + 103, 57 + 104, 57 + 105, 57 + 106, 57 + 108,
57 + 109, 57 + 110, 57 + 111, 60 + 102, 60 + 103, 60 + 104, 60 + 105, 60 + 106, 60 + 108, 60 + 109,
60 + 110, 60 + 111, 102 + 103, 102 + 104, 102 + 105, 102 + 106, 102 + 108, 102 + 109, 102 + 110,
102 + 111, 103 + 104, 103 + 105, 103 + 106, 103 + 108, 103 + 109, 103 + 110, 103 + 111, 104 + 105,
104 + 106, 104 + 108, 104 + 109, 104 + 110, 104 + 111, 105 + 106, 105 + 108, 105 + 109, 105 + 110,
105 + 111, 106 + 108, 106 + 109, 106 + 110, 106 + 111, 108 + 109, 108 + 110, 108 + 111, 109 + 110,
109 + 111, 110 + 111, 53 + 54 + 56, 53 + 54 + 57, 53 + 54 + 60, 53 + 54 + 102, 53 + 54 + 103, 53 + 54 + 104,
53 + 54 + 105, 53 + 54 + 106, 53 + 54 + 108, 53 + 54 + 109, 53 + 54 + 110, 53 + 54 + 111, 53 + 56 + 57,
53 + 56 + 60, 53 + 56 + 102, 53 + 56 + 103, 53 + 56 + 104, 53 + 56 + 105, 53 + 56 + 106, 53 + 56 + 108,
53 + 56 + 109, 53 + 56 + 110, 53 + 56 + 111, 53 + 57 + 60, 53 + 57 + 102, 53 + 57 + 103, 53 + 57 + 104,
53 + 57 + 105, 53 + 57 + 106, 53 + 57 + 108, 53 + 57 + 109, 53 + 57 + 110, 53 + 57 + 111, 53 + 60 + 102,
53 + 60 + 103, 53 + 60 + 104, 53 + 60 + 105, 53 + 60 + 106, 53 + 60 + 108, 53 + 60 + 109, 53 + 60 + 110,
53 + 60 + 111, 53 + 102 + 103, 53 + 102 + 104, 53 + 102 + 105, 53 + 102 + 106, 53 + 102 + 108,
53 + 102 + 109, 53 + 102 + 110, 53 + 102 + 111, 53 + 103 + 104, 53 + 103 + 105, 53 + 103 + 106,
53 + 103 + 108, 53 + 103 + 109, 53 + 103 + 110, 53 + 103 + 111, 53 + 104 + 105, 53 + 104 + 106,
53 + 104 + 108, 53 + 104 + 109, 53 + 104 + 110, 53 + 104 + 111, 53 + 105 + 106, 53 + 105 + 108,
53 + 105 + 109, 53 + 105 + 110, 53 + 105 + 111, 53 + 106 + 108, 53 + 106 + 109, 53 + 106 + 110,
53 + 106 + 111, 53 + 108 + 109, 53 + 108 + 110, 53 + 108 + 111, 53 + 109 + 110, 53 + 109 + 111,
53 + 110 + 111, 54 + 56 + 57, 54 + 56 + 60, 54 + 56 + 102, 54 + 56 + 103, 54 + 56 + 104, 54 + 56 + 105,
54 + 56 + 106, 54 + 56 + 108, 54 + 56 + 109, 54 + 56 + 110, 54 + 56 + 111, 54 + 57 + 60, 54 + 57 + 102,
54 + 57 + 103, 54 + 57 + 104, 54 + 57 + 105, 54 + 57 + 106, 54 + 57 + 108, 54 + 57 + 109, 54 + 57 + 110,
54 + 57 + 111, 54 + 60 + 102, 54 + 60 + 103, 54 + 60 + 104, 54 + 60 + 105, 54 + 60 + 106, 54 + 60 + 108,
54 + 60 + 109, 54 + 60 + 110, 54 + 60 + 111, 54 + 102 + 103, 54 + 102 + 104, 54 + 102 + 105, 54 + 102 + 106,
54 + 102 + 108, 54 + 102 + 109, 54 + 102 + 110, 54 + 102 + 111, 54 + 103 + 104, 54 + 103 + 105,
54 + 103 + 106, 54 + 103 + 108, 54 + 103 + 109, 54 + 103 + 110, 54 + 103 + 111, 54 + 104 + 105,
54 + 104 + 106, 54 + 104 + 108, 54 + 104 + 109, 54 + 104 + 110, 54 + 104 + 111, 54 + 105 + 106,
54 + 105 + 108, 54 + 105 + 109, 54 + 105 + 110, 54 + 105 + 111, 54 + 106 + 108, 54 + 106 + 109,
54 + 106 + 110, 54 + 106 + 111, 54 + 108 + 109, 54 + 108 + 110, 54 + 108 + 111, 54 + 109 + 110,
54 + 109 + 111, 54 + 110 + 111, 56 + 57 + 60, 56 + 57 + 102, 56 + 57 + 103, 56 + 57 + 104, 56 + 57 + 105,
56 + 57 + 106, 56 + 57 + 108, 56 + 57 + 109, 56 + 57 + 110, 56 + 57 + 111, 56 + 60 + 102, 56 + 60 + 103,
56 + 60 + 104, 56 + 60 + 105, 56 + 60 + 106, 56 + 60 + 108, 56 + 60 + 109, 56 + 60 + 110, 56 + 60 + 111,
56 + 102 + 103, 56 + 102 + 104, 56 + 102 + 105, 56 + 102 + 106, 56 + 102 + 108, 56 + 102 + 109,
56 + 102 + 110, 56 + 102 + 111, 56 + 103 + 104, 56 + 103 + 105, 56 + 103 + 106, 56 + 103 + 108,
56 + 103 + 109, 56 + 103 + 110, 56 + 103 + 111, 56 + 104 + 105, 56 + 104 + 106, 56 + 104 + 108,
56 + 104 + 109, 56 + 104 + 110, 56 + 104 + 111, 56 + 105 + 106, 56 + 105 + 108, 56 + 105 + 109,
56 + 105 + 110, 56 + 105 + 111, 56 + 106 + 108, 56 + 106 + 109, 56 + 106 + 110, 56 + 106 + 111,
56 + 108 + 109, 56 + 108 + 110, 56 + 108 + 111, 56 + 109 + 110, 56 + 109 + 111, 56 + 110 + 111,
57 + 60 + 102, 57 + 60 + 103, 57 + 60 + 104, 57 + 60 + 105, 57 + 60 + 106, 57 + 60 + 108, 57 + 60 + 109,
57 + 60 + 110, 57 + 60 + 111, 57 + 102 + 103, 57 + 102 + 104, 57 + 102 + 105, 57 + 102 + 106,
57 + 102 + 108, 57 + 102 + 109, 57 + 102 + 110, 57 + 102 + 111, 57 + 103 + 104, 57 + 103 + 105,
57 + 103 + 106, 57 + 103 + 108, 57 + 103 + 109, 57 + 103 + 110, 57 + 103 + 111, 57 + 104 + 105,
57 + 104 + 106, 57 + 104 + 108, 57 + 104 + 109, 57 + 104 + 110, 57 + 104 + 111, 57 + 105 + 106,
57 + 105 + 108, 57 + 105 + 109, 57 + 105 + 110, 57 + 105 + 111, 57 + 106 + 108, 57 + 106 + 109,
57 + 106 + 110, 57 + 106 + 111, 57 + 108 + 109, 57 + 108 + 110, 57 + 108 + 111, 57 + 109 + 110,
57 + 109 + 111, 57 + 110 + 111, 60 + 102 + 103, 60 + 102 + 104, 60 + 102 + 105, 60 + 102 + 106,
60 + 102 + 108, 60 + 102 + 109, 60 + 102 + 110, 60 + 102 + 111, 60 + 103 + 104, 60 + 103 + 105,
60 + 103 + 106, 60 + 103 + 108, 60 + 103 + 109, 60 + 103 + 110, 60 + 103 + 111, 60 + 104 + 105,
60 + 104 + 106, 60 + 104 + 108, 60 + 104 + 109, 60 + 104 + 110, 60 + 104 + 111, 60 + 105 + 106,
60 + 105 + 108, 60 + 105 + 109, 60 + 105 + 110, 60 + 105 + 111, 60 + 106 + 108, 60 + 106 + 109,
60 + 106 + 110, 60 + 106 + 111, 60 + 108 + 109, 60 + 108 + 110, 60 + 108 + 111, 60 + 109 + 110,
60 + 109 + 111, 60 + 110 + 111, 102 + 103 + 104, 102 + 103 + 105, 102 + 103 + 106, 102 + 103 + 108,
102 + 103 + 109, 102 + 103 + 110, 102 + 103 + 111, 102 + 104 + 105, 102 + 104 + 106, 102 + 104 + 108,
102 + 104 + 109, 102 + 104 + 110, 102 + 104 + 111, 102 + 105 + 106, 102 + 105 + 108, 102 + 105 + 109,
102 + 105 + 110, 102 + 105 + 111, 102 + 106 + 108, 102 + 106 + 109, 102 + 106 + 110, 102 + 106 + 111,
102 + 108 + 109, 102 + 108 + 110, 102 + 108 + 111, 102 + 109 + 110, 102 + 109 + 111, 102 + 110 + 111,
103 + 104 + 105, 103 + 104 + 106, 103 + 104 + 108, 103 + 104 + 109, 103 + 104 + 110, 103 + 104 + 111,
103 + 105 + 106, 103 + 105 + 108, 103 + 105 + 109, 103 + 105 + 110, 103 + 105 + 111, 103 + 106 + 108,
103 + 106 + 109, 103 + 106 + 110, 103 + 106 + 111, 103 + 108 + 109, 103 + 108 + 110, 103 + 108 + 111,
103 + 109 + 110, 103 + 109 + 111, 103 + 110 + 111, 104 + 105 + 106, 104 + 105 + 108, 104 + 105 + 109,
104 + 105 + 110, 104 + 105 + 111, 104 + 106 + 108, 104 + 106 + 109, 104 + 106 + 110, 104 + 106 + 111,
104 + 108 + 109, 104 + 108 + 110, 104 + 108 + 111, 104 + 109 + 110, 104 + 109 + 111, 104 + 110 + 111,
105 + 106 + 108, 105 + 106 + 109, 105 + 106 + 110, 105 + 106 + 111, 105 + 108 + 109, 105 + 108 + 110,
105 + 108 + 111, 105 + 109 + 110, 105 + 109 + 111, 105 + 110 + 111, 106 + 108 + 109, 106 + 108 + 110,
106 + 108 + 111, 106 + 109 + 110, 106 + 109 + 111, 106 + 110 + 111, 108 + 109 + 110, 108 + 109 + 111,
108 + 110 + 111, or 109 + 110 + 111

In some examples of any of the ABPCs described herein, an LCVD includes an LCVD that is at least 90% (e.g., at least 92%, at least 94%, at least 96%, at least 98%, at least 99%, or 100%) identical to SEQ ID NO: 127, where the LCVD includes a histidine at any of the specific combinations of one or more amino acid positions in SEQ ID NO: 127 listed in Table 4.

TABLE 4
Exemplary Combinations of Amino Acid Positions in SEQ ID NO: 127 that can be Substituted with Histidine

25 + 26, 25 + 28, 25 + 29, 25 + 31, 25 + 32, 25 + 33, 25 + 50, 25 + 90, 25 + 92, 25 + 94, 26 + 28, 26 + 29,
26 + 31, 26 + 32, 26 + 33, 26 + 50, 26 + 90, 26 + 92, 26 + 94, 28 + 29, 28 + 31, 28 + 32, 28 + 33, 28 + 50,
28 + 90, 28 + 92, 28 + 94, 29 + 31, 29 + 32, 29 + 33, 29 + 50, 29 + 90, 29 + 92, 29 + 94, 31 + 32, 31 + 33,
31 + 50, 31 + 90, 31 + 92, 31 + 94, 32 + 33, 32 + 50, 32 + 90, 32 + 92, 32 + 94, 33 + 50, 33 + 90, 33 + 92,
33 + 94, 50 + 90, 50 + 92, 50 + 94, 90 + 92, 90 + 94, 92 + 94, 25 + 92 + 94, 26 + 28 + 29, 26 + 28 + 31,
26 + 28 + 32, 26 + 28 + 33, 26 + 28 + 50, 26 + 28 + 90, 26 + 28 + 92, 26 + 28 + 94, 26 + 29 + 31, 26 + 29 + 32,
26 + 29 + 33, 26 + 29 + 50, 26 + 29-90, 26 + 29 + 92, 26 + 29 + 94, 26 + 31 + 32, 26 + 31 + 33, 26 + 31 + 50,
26 + 31 + 90, 26 + 31 + 92, 26 + 31 + 94, 26 + 32 + 33, 26 + 32 + 50, 26 + 32 + 90, 26 + 32 + 92, 26 + 32 + 94,
26 + 33 + 50, 26 + 33 + 90, 26 + 33 + 92, 26 + 33 + 94, 26 + 50 + 90, 26 + 50 + 92, 26 + 50 + 94, 26 + 90 + 92,
26 + 90 + 94, 26 + 92 + 94, 28 + 29 + 31, 28 + 29 + 32, 28 + 29 + 33, 28 + 29 + 50, 28 + 29 + 90, 28 + 29 + 92,
28 + 29 + 94, 28 + 31 + 32, 28 + 31 + 33, 28 + 31 + 50, 28 + 31 + 90, 28 + 31 + 92, 28 + 31 + 94, 28 + 32 + 33,
28 + 32 + 50, 28 + 32 + 90, 28 + 32 + 92, 28 + 32 + 94, 28 + 33 + 50, 28 + 33 + 90, 28 + 33 + 92, 28 + 33 + 94,
28 + 50 + 90, 28 + 50 + 92, 28 + 50 + 94, 28 + 90 + 92, 28 + 90 + 94, 28 + 92 + 94, 29 + 31 + 32, 29 + 31 + 33,
29 + 31 + 50, 29 + 31 + 90, 29 + 31 + 92, 29 + 31 + 94, 29 + 32 + 33, 29 + 32 + 50, 29 + 32 + 90, 29 + 32 + 92,
29 + 32 + 94, 29 + 33 + 50, 29 + 33 + 90, 29 + 33 + 92, 29 + 33 + 94, 29 + 50 + 90, 29 + 50 + 92, 29 + 50 + 94,
29 + 90 + 92, 29 + 90 + 94, 29 + 92 + 94, 31 + 32 + 33, 31 + 32 + 50, 31 + 32 + 90, 31 + 32 + 92, 31 + 32 + 94,
31 + 33 + 50, 31 + 33 + 90, 31 + 33 + 92, 31 + 33 + 94, 31 + 50 + 90, 31 + 50 + 92, 31 + 50 + 94, 31 + 90 + 92,
31 + 90 + 94, 31 + 92 + 94, 32 + 33 + 50, 32 + 33 + 90, 32 + 33 + 92, 32 + 33 + 94, 32 + 50 + 90, 32 + 50 + 92,
32 + 50 + 94, 32 + 90 + 92, 32 + 90 + 94, 32 + 92 + 94, 33 + 50 + 90, 33 + 50 + 92, 33 + 50 + 94, 33 + 90 + 92,
33 + 90 + 94, 33 + 92 + 94, 50 + 90 + 92, 50 + 90 + 94, 50 + 92 + 94, or 90 + 92 + 94

In some examples of any of the ABPCs described herein, the first ABD includes an HCVD that is at least 90% (e.g., at least 92%, at least 94%, at least 96%, at least 98%, at least 99%, or 100%) identical to SEQ ID NO: 126, where the HCVD includes a histidine at any of the specific combinations of one or more amino acid positions in SEQ ID NO: 126 listed in Table 3; and an LCVD that that is at least 90% (e.g., at least 92%, at least 94%, at least 96%, at least 98%, at least 99%, or 100%) identical to SEQ ID NO: 127, where the LCVD includes a histidine at any of the specific combinations of one or more amino acid positions in SEQ ID NO: 127 listed in Table 4.

In some examples of any of the ABPCs described herein, the first ABD comprises an LCVD comprising SEQ ID NO: 127, and an HCVD that is at least 90% identical (e.g., ≥92%, ≥94%, ≥96%, ≥98%, ≥99%, or 100%) identical to SEQ ID NO: 126, where the HCVD includes a histidine at any of the specific combinations of one or more (e.g., two, three, four, five, six, seven, eight, nine, or ten) amino acid positions in SEQ ID NO: 126 listed in Table 3.

In some examples of any of the ABPCs described herein, the first ABD comprises an LCVD that is at least 90% identical to SEQ ID NO: 127, wherein the LCVD includes a histidine at position 25 in SEQ ID NO: 127; and an HCVD that is at least 90% (e.g., at least 92%, at least 94%, at least 96%, at least 98%, at least 99%, or 100%) identical to SEQ ID NO: 126, where the HCVD includes a histidine at any of the specific combinations of one or more amino acid positions in SEQ ID NO: 126 listed in Table 3.

In some examples of any of the ABPCs described herein, the first ABD comprises an LCVD that is at least 90% identical to SEQ ID NO: 127, wherein the LCVD includes a histidine at position 26 in SEQ ID NO: 127; and an HCVD that is at least 90% (e.g., at least 92%, at least 94%, at least 96%, at least 98%, at least 99%, or 100%) identical to SEQ ID NO: 126, where the HCVD includes a histidine at any of the specific combinations of one or more amino acid positions in SEQ ID NO: 126 listed in Table 3.

In some examples of any of the ABPCs described herein, the first ABD comprises an LCVD that is at least 90% identical to SEQ ID NO: 127, wherein the LCVD includes a histidine at position 28 in SEQ ID NO: 127; and an HCVD that is at least 90% (e.g., at least 92%, at least 94%, at least 96%, at least 98%, at least 99%, or 100%) identical to SEQ ID NO: 126, where the HCVD includes a histidine at any of the specific combinations of one or more amino acid positions in SEQ ID NO: 126 listed in Table 3.

In some examples of any of the ABPCs described herein, the first ABD comprises an LCVD that is at least 90% identical to SEQ ID NO: 127, wherein the LCVD includes a histidine at position 29 in SEQ ID NO: 127; and an HCVD that is at least 90% (e.g., at least 92%, at least 94%, at least 96%, at least 98%, at least 99%, or 100%) identical to SEQ ID NO: 126, where the HCVD includes a histidine at any of the specific combinations of one or more amino acid positions in SEQ ID NO: 126 listed in Table 3.

In some examples of any of the ABPCs described herein, the first ABD comprises an LCVD that is at least 90% identical to SEQ ID NO: 127, wherein the LCVD includes a histidine at position 31 in SEQ ID NO: 127; and an HCVD that is at least 90% (e.g., at least 92%, at least 94%, at least 96%, at least 98%, at least 99%, or 100%) identical to SEQ ID NO: 126, where the HCVD includes a histidine at any of the specific combinations of one or more amino acid positions in SEQ ID NO: 126 listed in Table 3.

In some examples of any of the ABPCs described herein, the first ABD comprises an LCVD that is at least 90% identical to SEQ ID NO: 127, wherein the LCVD includes a histidine at position 32 in SEQ ID NO: 127; and an HCVD that is at least 90% (e.g., at least 92%, at least 94%, at least 96%, at least 98%, at least 99%, or 100%) identical to SEQ ID NO: 126, where the HCVD includes a histidine at any of the specific combinations of one or more amino acid positions in SEQ ID NO: 126 listed in Table 3.

In some examples of any of the ABPCs described herein, the first ABD comprises an LCVD that is at least 90% identical to SEQ ID NO: 127, wherein the LCVD includes a histidine at position 33 in SEQ ID NO: 127; and an HCVD that is at least 90% (e.g., at least 92%, at least 94%, at least 96%, at least 98%, at least 99%, or 100%) identical to SEQ ID NO: 126, where the HCVD includes a histidine at any of the specific combinations of one or more amino acid positions in SEQ ID NO: 126 listed in Table 3.

In some examples of any of the ABPCs described herein, the first ABD comprises an LCVD that is at least 90% identical to SEQ ID NO: 127, wherein the LCVD includes a histidine at position 50 in SEQ ID NO: 127; and an HCVD that is at least 90% (e.g., at least 92%, at least 94%, at least 96%, at least 98%, at least 99%, or 100%) identical to SEQ ID NO: 126, where the HCVD includes a histidine at any of the specific combinations of one or more amino acid positions in SEQ ID NO: 126 listed in Table 3.

In some examples of any of the ABPCs described herein, the first ABD comprises an LCVD that is at least 90% identical to SEQ ID NO: 127, wherein the LCVD includes a histidine at position 90 in SEQ ID NO: 127; and an HCVD that is at least 90% (e.g., at least 92%, at least 94%, at least 96%, at least 98%, at least 99%, or 100%) identical to SEQ ID NO: 126, where the HCVD includes a histidine at any of the specific combinations of one or more amino acid positions in SEQ ID NO: 126 listed in Table 3.

In some examples of any of the ABPCs described herein, the first ABD comprises an LCVD that is at least 90% identical to SEQ ID NO: 127, wherein the LCVD includes a histidine at position 92 in SEQ ID NO: 127; and an HCVD that is at least 90% (e.g., at least 92%, at least 94%, at least 96%, at least 98%, at least 99%, or 100%) identical to SEQ ID NO: 126, where the HCVD includes a histidine at any of the specific combinations of one or more amino acid positions in SEQ ID NO: 126 listed in Table 3.

In some examples of any of the ABPCs described herein, the first ABD comprises an LCVD that is at least 90% identical to SEQ ID NO: 127, wherein the LCVD includes a histidine at position 94 in SEQ ID NO: 127; and an HCVD that is at least 90% (e.g., at least 92%, at least 94%, at least 96%, at least 98%, at least 99%, or 100%) identical to SEQ ID NO: 126, where the HCVD includes a histidine at any of the specific combinations of one or more amino acid positions in SEQ ID NO: 126 listed in Table 3.

In some examples of any of the ABPCs described herein, the first ABD includes an HCVD of 7C8 with one or more histidine(s) substituted with an alanine. In some examples of any of the ABPCs described herein, the first ABD includes an LCVD of 7C8 with one or more histidine(s) substituted with an alanine. In some examples of any of the ABPCs described herein, the first ABD includes an HCVD of 7C8 with one or more histidines substituted with an alanine; and an LCVD of 7C8 with one or more histidine(s) substituted with an alanine. In some examples of any of the ABPCs described herein, the HCVD of 7C8 comprises SEQ ID NO: 126. In some examples of any of the ABPCs described herein, the LCVD of 7C8 comprises SEQ ID NO: 127.

In some examples of any of the ABPCs described herein, the first ABD comprises an HCVD comprising a CDR1, a CDR2, and a CDR3 of SEQ ID NO: 128, SEQ ID NO: 129, and SEQ ID NO: 130, respectively, with collectively a total of one or more (e.g., one, two, three, four, five, six, seven, eight, nine, or ten) histidine(s) in SEQ ID NOs: 128, 129, and 130 substituted with an alanine. In some examples of any of the ABPCs described herein, the first ABD comprises an LCVD comprising a CDR1, a CDR2, and a CDR3 of SEQ ID NO: 131, SEQ ID NO: 132, and SEQ ID NO: 133, respectively, with collectively a total of one or more (e.g., one, two, three, four, five, six, seven, eight, nine, or ten) histidine(s) in SEQ ID NOs: 131, 132, and 133 substituted with an alanine. In some examples of any of the ABPCs described herein, the first ABD includes: an HCVD comprising a CDR1, a CDR2, and a CDR3 of SEQ ID NO: 128, SEQ ID NO: 129, and SEQ ID NO: 130, respectively, with collectively a total of one or more (e.g., one, two, three, four, five, six, seven, eight, nine, or ten) histidine(s) in SEQ ID NOs: 128, 129, and 130 substituted with an alanine; and an LCVD comprising a CDR1, a CDR2, and a CDR3 of SEQ ID NO: 131, SEQ ID NO: 132, and SEQ ID NO: 133, respectively, with collectively a total of one or more (e.g., one, two, three, four, five, six, seven, eight, nine, or ten) histidine(s) in SEQ ID NOs: 131, 132, and 133 substituted with an alanine.

In some examples of any of the ABPCs described herein, the first ABD comprises an HCVD of SEQ ID NOS: 126, or one of 134-179, or one of 208-227.

In some examples of any of the ABPCs described herein, the first ABD includes an LCVD of SEQ ID NO: 127, or one of 180-207, or 228.

In some examples of any of the ABPCs described herein, the first ABD includes an LCVD comprising SEQ ID NO: 127, and an HCVD comprising one of SEQ ID NOs: 126, or one of 134-179, or one of 208-227. In some examples of any of the ABPCs described herein, the first ABD includes an LCVD comprising SEQ ID NO: 180, and an HCVD comprising SEQ ID NOS: 126, or one of 134-179, or one of 208-227. In some examples of any of the ABPCs described herein, the first ABD includes an LCVD comprising SEQ ID NO: 181, and an HCVD comprising SEQ ID NOS: 126, or one of 134-179, or one of 208-227. In some examples of any of the ABPCs described herein, the first ABD includes an LCVD comprising SEQ ID NO: 182, and an HCVD comprising SEQ ID NOS: 126, or one of 134-179, or one of 208-227. In some examples of any of the ABPCs described herein, the first ABD includes an LCVD comprising SEQ ID NO: 183, and an HCVD comprising SEQ ID NOS: 126, or one of 134-179, or one of 208-227. In some examples of any of the ABPCs described herein, the first ABD includes an LCVD comprising SEQ ID NO: 184, and an HCVD comprising SEQ ID NOS: 126, or one of 134-179, or one of 208-227. In some examples of any of the ABPCs described herein, the first ABD includes an LCVD comprising SEQ ID NO: 185, and an HCVD comprising SEQ ID NOS: 126, or one of 134-179, or one of 208-227. In some examples of any of the ABPCs described herein, the first ABD includes an LCVD comprising SEQ ID NO: 186, and an HCVD comprising SEQ ID NOS: 126, or one of 134-179, or one of 208-227. In some examples of any of the ABPCs described herein, the first ABD includes an LCVD comprising SEQ ID NO: 187, and an HCVD comprising SEQ ID NOS: 126, or one of 134-179, or one of 208-227. In some examples of any of the ABPCs described herein, the first ABD includes an LCVD comprising SEQ ID NO: 188, and an HCVD comprising SEQ ID NOS: 126, or one of 134-179, or one of 208-227. In some examples of any of the ABPCs described herein, the first ABD includes an LCVD comprising SEQ ID NO: 189, and an HCVD comprising SEQ ID NOS: 126, or one of 134-179, or one of 208-227. In some examples of any of the ABPCs described herein, the first ABD includes an LCVD comprising SEQ ID NO: 190, and an HCVD comprising SEQ ID NOS: 126, or one of 134-179, or one of 208-227. In some examples of any of the ABPCs described herein, the first ABD includes an LCVD comprising SEQ ID NO: 191, and an HCVD comprising SEQ ID NOS: 126, or one of 134-179, or one of 208-227. In some examples of any of the ABPCs described herein, the first ABD includes an LCVD comprising SEQ ID NO: 192, and an HCVD comprising SEQ ID NOS: 126, or one of 134-179, or one of 208-227. In some examples of any of the ABPCs described herein, the first ABD includes an LCVD comprising SEQ ID NO: 193, and an HCVD comprising SEQ ID NOS: 126, or one of 134-179, or one of 208-227. In some examples of any of the ABPCs described herein, the first ABD includes an LCVD comprising SEQ ID NO: 194, and an HCVD comprising SEQ ID NOS: 126, or one of 134-179, or one of 208-227. In some examples of any of the ABPCs described herein, the first ABD includes an LCVD comprising SEQ ID NO: 195, and an HCVD comprising SEQ ID NOS: 126, or one of 134-179, or one of 208-227. In some examples of any of the ABPCs described herein, the first ABD includes an LCVD comprising SEQ ID NO: 196, and an HCVD comprising SEQ ID NOS: 126, or one of 134-179, or one of 208-227. In some examples of any of the ABPCs described herein, the first ABD includes an LCVD comprising SEQ ID NO: 197, and an HCVD comprising SEQ ID NOS: 126, or one of 134-179, or one of 208-227. In some examples of any of the ABPCs described herein, the first ABD includes an LCVD comprising SEQ ID NO: 198, and an HCVD comprising SEQ ID NOS: 126, or one of 134-179, or one of 208-227. In some examples of any of the ABPCs described herein, the first ABD includes an LCVD comprising SEQ ID NO: 199, and an HCVD comprising SEQ ID NOS: 126, or one of 134-179, or one of 208-227. In some examples of any of the ABPCs described herein, the first ABD includes an LCVD comprising SEQ ID NO: 200, and an HCVD comprising SEQ ID NOS: 126, or one of 134-179, or one of 208-227. In some examples of any of the ABPCs described herein, the first ABD includes an LCVD comprising SEQ ID NO: 201, and an HCVD comprising SEQ ID NOS: 126, or one of 134-179, or one of 208-227. In some examples of any of the ABPCs described herein, the first ABD includes an LCVD comprising SEQ ID NO: 202, and an HCVD comprising SEQ ID NOS: 126, or one of 134-179, or one of 208-227. In some examples of any of the ABPCs described herein, the first ABD includes an LCVD comprising SEQ ID NO: 203, and an HCVD comprising SEQ ID NOS: 126, or one of 134-179, or one of 208-227. In some examples of any of the ABPCs described herein, the first ABD includes an LCVD comprising SEQ ID NO: 204, and an HCVD comprising SEQ ID NOS: 126, or one of 134-179, or one of 208-227. In some examples of any of the ABPCs described herein, the first ABD includes an LCVD comprising SEQ ID NO: 205, and an HCVD comprising SEQ ID NOS: 126, or one of 134-179, or one of 208-227. In some examples of any of the ABPCs described herein, the first ABD includes an LCVD comprising SEQ ID NO: 206, and an HCVD comprising SEQ ID NOS: 126, or one of 134-179, or one of 208-227. In some examples of any of the ABPCs described herein, the first ABD includes an LCVD comprising SEQ ID NO: 207, and an HCVD comprising SEQ ID NOS: 126, or one of 134-179, or one of 208-227. In some examples of any of the ABPCs described herein, the first ABD includes an LCVD comprising SEQ ID NO: 228, and an HCVD comprising SEQ ID NOS: 126, or one of 134-179, or one of 208-227.

In some examples of any of the ABPCs described herein, the first ABD includes an HCVD of 12C6 with one or more amino acids substituted with a histidine. In some examples of any of the ABPCs described herein, the first ABD includes an LCVD of 12C6 with one or more amino acids substituted with a histidine. In some examples of any of the ABPCs described herein, the first ABD includes an HCVD of 12C6 with one or more amino acids substituted with a histidine; and an LCVD of 12C6 with one or more amino acids substituted with a histidine. In some examples of any of the ABPCs described herein, the HCVD of 12C6 comprises SEQ ID NO: 229. In some examples of any of the ABPCs described herein, the LCVD of 12C6 comprises SEQ ID NO: 230.

In some examples of any of the ABPCs described herein, the first ABD comprises an HCVD comprising a CDR1, a CDR2, and a CDR3 of SEQ ID NO: 231, 232, and 233, respectively, with collectively a total of one or more (e.g., one, two, three, four, five, six, seven, eight, nine, or ten) amino acid positions in SEQ ID NOs: 231, 232, and 233 substituted with a histidine. In some examples of any of the ABPCs described herein, the first ABD comprises an LCVD comprising a CDR1, a CDR2, and a CDR3 of SEQ ID NOs: 234, 235, and 236, respectively, with collectively a total of one or more (e.g., one, two, three, four, five, six, seven, eight, nine, or ten) amino acid positions in SEQ ID NOs: 234, 235, and 236 substituted with a histidine. In some examples of any of the ABPCs described herein, the first ABD includes: an HCVD comprising a CDR1, a CDR2, and a CDR3 of SEQ ID NO: 231, SEQ ID NO: 232, and SEQ ID NO: 233, respectively, with collectively a total of one or more (e.g., one, two, three, four, five, six, seven, eight, nine, or ten) amino acid positions in SEQ ID NOs: 231, 232, and 233 substituted with a histidine; and an LCVD comprising a CDR1, a CDR2, and a CDR3 of SEQ ID NO: 234, SEQ ID NO: 235, and SEQ ID NO: 236, respectively, with collectively a total of one or more (e.g., one, two, three, four, five, six, seven, eight, nine, or ten) amino acid positions in SEQ ID NOs: 234, 235, and 236 substituted with a histidine.

In some examples of any of the ABPCs described herein, the first ABD includes an HCVD that is at least 90% (e.g., at least 92%, at least 94%, at least 96%, at least 98%, at least 99%, or 100%) identical to SEQ ID NO: 229, where the HCVD includes a histidine at one or more amino acid positions in SEQ ID NO: 229 selected from the group of: 27, 32, 33, 34, 35, 51, 54, 56, 58, and 100. In some examples of any of the ABPCs described herein, the first ABD includes an LCVD that is at least 90% (e.g., at least 92%, at least 94%, at least 96%, at least 98%, at least 99%, or 100%) identical to SEQ ID NO: 230, where the LCVD includes a histidine at one or more amino acid positions in SEQ ID NO: 230 selected from the group of: 30, 31, 33, 51, 53, 57, 91, 92, 94, and 99. In some examples of any of the ABPCs described herein the first ABD includes: an HCVD that is at least 90% (e.g., at least 92%, at least 94%, at least 96%, at least 98%, at least 99%, or 100%) identical to SEQ ID NO: 229, where the HCVD includes a histidine at one or more amino acid positions in SEQ ID NO: 229 selected from the group consisting of: 27, 32, 33, 34, 35, 51, 54, 56, 58, and 100, and an LCVD that is at least 90% (e.g., at least 92%, at least 94%, at least 96%, at least 98%, at least 99%, or 100%) identical to SEQ ID NO: 230, where the LCVD includes a histidine at one or more amino acid positions in SEQ TD NO: 230 selected from the group consisting of: 30, 31, 33, 51, 53, 57, 91, 92, 94, and 99.

In some examples of any of the ABPCs described herein, an HCVD includes an HCVD that is at least 90 (e.g., at least 9200, at least 940, at least 96, at least 985, at least 99, or 1000%) identical to SEQ TD NO: 229, where the HCVD includes a histidine at any of the specific combinations of one or more amino acid positions in SEQ ID NO: 229 listed in Table 5.

TABLE 5
Exemplary Combinations of Amino Acid Positions in SEQ ID NO: 229 that can be Substituted with Histidine

27 + 32, 27 + 33, 27 + 34, 27 + 35, 27 + 51, 27 + 54, 27 + 56, 27 + 58, 27 + 100, 32 + 33, 32 + 34, 32 + 35,
32 + 51, 32 + 54, 32 + 56, 32 + 58, 32 + 100, 33 + 34, 33 + 35, 33 + 51, 33 + 54, 33 + 56, 33 + 58, 33 + 100,
34 + 35, 34 + 51, 34 + 54, 34 + 56, 34 + 58, 34 + 100, 35 + 51, 35 + 54, 35 + 56, 35 + 58, 35 + 100, 51 + 54,
51 + 56, 51 + 58, 51 + 100, 54 + 56, 54 + 58, 54 + 100, 56 + 58, 56 + 100, 58 + 100, 27 + 32 + 33,
27 + 32 + 34, 27 + 32 + 35, 27 + 32 + 51, 27 + 32 + 54, 27 + 32 + 56, 27 + 32 + 58, 27 + 32 + 100, 27 + 33 + 34,
27 + 33 + 35, 27 + 33 + 51, 27 + 33 + 54, 27 + 33 + 56, 27 + 33 + 58, 27 + 33 + 100, 27 + 34 + 35, 27 + 34 + 51,
27 + 34 + 54, 27 + 34 + 56, 27 + 34 + 58, 27 + 34 + 100, 27 + 35 + 51, 27 + 35 + 54, 27 + 35 + 56, 27 + 35 + 58,
27 + 35 + 100, 27 + 51 + 54, 27 + 51 + 56, 27 + 51 + 58, 27 + 51 + 100, 27 + 54 + 56, 27 + 54 + 58,
27 + 54 + 100, 27 + 56 + 58, 27 + 56 + 100, 27 + 58 + 100, 32 + 33 + 34, 32 + 33 + 35, 32 + 33 + 51,
32 + 33 + 54, 32 + 33 + 56, 32 + 33 + 58, 32 + 33 + 100, 32 + 34 + 35, 32 + 34 + 51, 32 + 34 + 54, 32 + 34 + 56,
32 + 34 + 58, 32 + 34 + 100, 32 + 35 + 51, 32 + 35 + 54, 32 + 35 + 56, 32 + 35 + 58, 32 + 35 + 100, 32 + 51 + 54,
32 + 51 + 56, 32 + 51 + 58, 32 + 51 + 100, 32 + 54 + 56, 32 + 54 + 58, 32 + 54 + 100, 32 + 56 + 58,
32 + 56 + 100, 32 + 58 + 100, 33 + 34 + 35, 33 + 34 + 51, 33 + 34 + 54, 33 + 34 + 56, 33 + 34 + 58,
33 + 34 + 100, 33 + 35 + 51, 33 + 35 + 54, 33 + 35 + 56, 33 + 35 + 58, 33 + 35 + 100, 33 + 51 + 54, 33 + 51 + 56,
33 + 51 + 58, 33 + 51 + 100, 33 + 54 + 56, 33 + 54 + 58, 33 + 54 + 100, 33 + 56 + 58, 33 + 56 + 100,
33 + 58 + 100, 34 + 35 + 51, 34 + 35 + 54, 34 + 35 + 56, 34 + 35 + 58, 34 + 35 + 100, 34 + 51 + 54, 34 + 51 + 56,
34 + 51 + 58, 34 + 51 + 100, 34 + 54 + 56, 34 + 54 + 58, 34 + 54 + 100, 34 + 56 + 58, 34 + 56 + 100,
34 + 58 + 100, 35 + 51 + 54, 35 + 51 + 56, 35 + 51 + 58, 35 + 51 + 100, 35 + 54 + 56, 35 + 54 + 58,
35 + 54 + 100, 35 + 56 + 58, 35 + 56 + 100, 35 + 58 + 100, 51 + 54 + 56, 51 + 54 + 58, 51 + 54 + 100,
51 + 56 + 58, 51 + 56 + 100, 51 + 58 + 100, 54 + 56 + 58, 54 + 56 + 100, 54 + 58 + 100, or 56 + 58 + 100

In some examples of any of the ABPCs described herein, an LCVD includes an LCVD that is at least 90% a (e.g., at least 920%, at least 9400 at least 96%, at least 98%, at least 99%, or 1000%) identical to SEQ TD NO: 230, where the LCVD includes a histidine at any of the specific combinations of one or more amino acid positions in SEQ TD NO: 230 listed in Table 6.

TABLE 6
Exemplary Combinations of Amino Acid Positions in SEQ ID NO: 230 that can be Substituted with Histidine

30 + 31, 30 + 33, 30 + 51, 30 + 53, 30 + 57, 30 + 91, 30 + 92, 30 + 94, 30 + 99, 31 + 33, 31 + 51, 31 + 53,
31 + 57, 31 + 91, 31 + 92, 31 + 94, 31 + 99, 33 + 51, 33 + 53, 33 + 57, 33 + 91, 33 + 92, 33 + 94, 33 + 99,
51 + 53, 51 + 57, 51 + 91, 51 + 92, 51 + 94, 51 + 99, 53 + 57, 53 + 91, 53 + 92, 53 + 94, 53 + 99, 57 + 91,
57 + 92, 57 + 94, 57 + 99, 91 + 92, 91 + 94, 91 + 99, 92 + 94, 92 + 99, 94 + 99, 30 + 31 + 33, 30 + 31 + 51,
30 + 31 + 53, 30 + 31 + 57, 30 + 31 + 91, 30 + 31 + 92, 30 + 31 + 94, 30 + 31 + 99, 30 + 33 + 51, 30 + 33 + 53,
30 + 33 + 57, 30 + 33 + 91, 30 + 33 + 92, 30 + 33 + 94, 30 + 33 + 99, 30 + 51 + 53, 30 + 51 + 57, 30 + 51 + 91,
30 + 51 + 92, 30 + 51 + 94, 30 + 51 + 99, 30 + 53 + 57, 30 + 53 + 91, 30 + 53 + 92, 30 + 53 + 94, 30 + 53 + 99,
30 + 57 + 91, 30 + 57 + 92, 30 + 57 + 94, 30 + 57 + 99, 30 + 91 + 92, 30 + 91 + 94, 30 + 91 + 99, 30 + 92 + 94,
30 + 92 + 99, 30 + 94 + 99, 31 + 33 + 51, 31 + 33 + 53, 31 + 33 + 57, 31 + 33 + 91, 31 + 33 + 92, 31 + 33 + 94,
31 + 33 + 99, 31 + 51 + 53, 31 + 51 + 57, 31 + 51 + 91, 31 + 51 + 92, 31 + 51 + 94, 31 + 51 + 99, 31 + 53 + 57,
31 + 53 + 91, 31 + 53 + 92, 31 + 53 + 94, 31 + 53 + 99, 31 + 57 + 91, 31 + 57 + 92, 31 + 57 + 94, 31 + 57 + 99,
31 + 91 + 92, 31 + 91 + 94, 31 + 91 + 99, 31 + 92 + 94, 31 + 92 + 99, 31 + 94 + 99, 33 + 51 + 53, 33 + 51 + 57,
33 + 51 + 91, 33 + 51 + 92, 33 + 51 + 94, 33 + 51 + 99, 33 + 53 + 57, 33 + 53 + 91, 33 + 53 + 92, 33 + 53 + 94,
33 + 53 + 99, 33 + 57 + 91, 33 + 57 + 92, 33 + 57 + 94, 33 + 57 + 99, 33 + 91 + 92, 33 + 91 + 94, 33 + 91 + 99,
33 + 92 + 94, 33 + 92 + 99, 33 + 94 + 99, 51 + 53 + 57, 51 + 53 + 91, 51 + 53 + 92, 51 + 53 + 94, 51 + 53 + 99,
51 + 57 + 91, 51 + 57 + 92, 51 + 57 + 94, 51 + 57 + 99, 51 + 91 + 92, 51 + 91 + 94, 51 + 91 + 99, 51 + 92 + 94,
51 + 92 + 99, 51 + 94 + 99, 53 + 57 + 91, 53 + 57 + 92, 53 + 57 + 94, 53 + 57 + 99, 53 + 91 + 92, 53 + 91 + 94,
53 + 91 + 99, 53 + 92 + 94, 53 + 92 + 99, 53 + 94 + 99, 57 + 91 + 92, 57 + 91 + 94, 57 + 91 + 99, 57 + 92 + 94,
57 + 92 + 99, 57 + 94 + 99, 91 + 92 + 94, 91 + 92 + 99, 91 + 94 + 99, or 92 + 94 + 99

In some examples of any of the ABPCs described herein, the first ABD includes an HCVD that is at least 90% (e.g., at least 92%, at least 9400, at least 96%, at least 98%, at least 99%, or 100%) identical to SEQ ID NO: 229, where the HCVD includes a histidine at any of the specific combinations of one or more amino acid positions in SEQ ID NO: 229 listed in Table 5; and an LCVD that that is at least 90% (e.g., at least 92%, at least 94% at least 96%, at least 98%, at least 99%, or 100%) identical to SEQ ID NO: 230, where the LCVD includes a histidine at any of the specific combinations of one or more amino acid positions in SEQ ID NO: 230 listed in Table 6.

In some examples of any of the ABPCs described herein, the first ABD comprises an LCVD comprising SEQ ID NO: 230, and an HCVD that is at least 90% identical (e.g., at least 92%, at least 94%, at least 96%, at least 98%, at least 99%, or 100%) identical to SEQ ID NO: 229, where the HCVD includes a histidine at any of the specific combinations of one or more (e.g., two, three, four, five, six, seven, eight, nine, or ten) amino acid positions in SEQ ID NO: 229 listed in Table 5.

In some examples of any of the ABPCs described herein, the first ABD comprises an LCVD that is at least 90% identical to SEQ ID NO: 230, wherein the LCVD includes a histidine at position 30 in SEQ ID NO: 230; and an HCVD that is at least 90% (e.g., at least 92%, at least 94%, at least 96%, at least 98%, at least 99%, or 100%) identical to SEQ ID NO: 229, where the HCVD includes a histidine at any of the specific combinations of one or more amino acid positions in SEQ ID NO: 229 listed in Table 5.

In some examples of any of the ABPCs described herein, the first ABD comprises an LCVD that is at least 90% identical to SEQ ID NO: 230, wherein the LCVD includes a histidine at position 31 in SEQ ID NO: 230; and an HCVD that is at least 90% (e.g., at least 92%, at least 94%, at least 96%, at least 98%, at least 99%, or 100%) identical to SEQ ID NO: 229, where the HCVD includes a histidine at any of the specific combinations of one or more amino acid positions in SEQ ID NO: 229 listed in Table 5.

In some examples of any of the ABPCs described herein, the first ABD comprises an LCVD that is at least 90% identical to SEQ ID NO: 230, wherein the LCVD includes a histidine at position 33 in SEQ ID NO: 230; and an HCVD that is at least 90% (e.g., at least 92%, at least 94%, at least 96%, at least 98%, at least 99%, or 100%) identical to SEQ ID NO: 229, where the HCVD includes a histidine at any of the specific combinations of one or more amino acid positions in SEQ ID NO: 229 listed in Table 5.

In some examples of any of the ABPCs described herein, the first ABD comprises an LCVD that is at least 90% identical to SEQ ID NO: 230, wherein the LCVD includes a histidine at position 51 in SEQ ID NO: 230; and an HCVD that is at least 90% (e.g., at least 92%, at least 94%, at least 96%, at least 98%, at least 99%, or 100%) identical to SEQ ID NO: 229, where the HCVD includes a histidine at any of the specific combinations of one or more amino acid positions in SEQ ID NO: 229 listed in Table 5.

In some examples of any of the ABPCs described herein, the first ABD comprises an LCVD that is at least 90% identical to SEQ ID NO: 230, wherein the LCVD includes a histidine at position 53 in SEQ ID NO: 230; and an HCVD that is at least 90% (e.g., at least 92%, at least 94%, at least 96%, at least 98%, at least 99%, or 100%) identical to SEQ ID NO: 229, where the HCVD includes a histidine at any of the specific combinations of one or more amino acid positions in SEQ ID NO: 229 listed in Table 5.

In some examples of any of the ABPCs described herein, the first ABD comprises an LCVD that is at least 90% identical to SEQ ID NO: 230, wherein the LCVD includes a histidine at position 57 in SEQ ID NO: 230; and an HCVD that is at least 90% (e.g., at least 92%, at least 94%, at least 96%, at least 98%, at least 99%, or 100%) identical to SEQ ID NO: 229, where the HCVD includes a histidine at any of the specific combinations of one or more amino acid positions in SEQ ID NO: 229 listed in Table 5.

In some examples of any of the ABPCs described herein, the first ABD comprises an LCVD that is at least 90% identical to SEQ ID NO: 230, wherein the LCVD includes a histidine at position 91 in SEQ ID NO: 230; and an HCVD that is at least 90% (e.g., at least 92%, at least 94%, at least 96%, at least 98%, at least 99%, or 100%) identical to SEQ ID NO: 229, where the HCVD includes a histidine at any of the specific combinations of one or more amino acid positions in SEQ ID NO: 229 listed in Table 5.

In some examples of any of the ABPCs described herein, the first ABD comprises an LCVD that is at least 90% identical to SEQ ID NO: 230, wherein the LCVD includes a histidine at position 92 in SEQ ID NO: 230; and an HCVD that is at least 90% (e.g., at least 92%, at least 94%, at least 96%, at least 98%, at least 99%, or 100%) identical to SEQ ID NO: 229, where the HCVD includes a histidine at any of the specific combinations of one or more amino acid positions in SEQ ID NO: 229 listed in Table 5.

In some examples of any of the ABPCs described herein, the first ABD comprises an LCVD that is at least 90% identical to SEQ ID NO: 230, wherein the LCVD includes a histidine at position 94 in SEQ ID NO: 230; and an HCVD that is at least 90% (e.g., at least 92%, at least 94%, at least 96%, at least 98%, at least 99%, or 100%) identical to SEQ ID NO: 229, where the HCVD includes a histidine at any of the specific combinations of one or more amino acid positions in SEQ ID NO: 229 listed in Table 5.

In some examples of any of the ABPCs described herein, the first ABD comprises an LCVD that is at least 90% identical to SEQ ID NO: 230, wherein the LCVD includes a histidine at position 99 in SEQ ID NO: 230; and an HCVD that is at least 90% (e.g., at least 92%, at least 94%, at least 96%, at least 98%, at least 99%, or 100%) identical to SEQ ID NO: 229, where the HCVD includes a histidine at any of the specific combinations of one or more amino acid positions in SEQ ID NO: 229 listed in Table 5.

In some examples of any of the ABPCs described herein, the first ABD includes an HCVD of 12C6 with one or more histidine(s) substituted with an alanine. In some examples of any of the ABPCs described herein, the first ABD includes an LCVD of 12C6 with one or more histidine(s) substituted with an alanine. In some examples of any of the ABPCs described herein, the first ABD includes an HCVD of 12C6 with one or more histidines substituted with an alanine; and an LCVD of 12C6 with one or more histidine(s) substituted with an alanine. In some examples of any of the ABPCs described herein, the HCVD of 12C6 comprises SEQ ID NO: 229. In some examples of any of the ABPCs described herein, the LCVD of 12C6 comprises SEQ ID NO: 230.

In some examples of any of the ABPCs described herein, the first ABD comprises an HCVD comprising a CDR1, a CDR2, and a CDR3 of SEQ ID NO: 231, SEQ ID NO: 232, and SEQ ID NO: 233, respectively, with collectively a total of one or more (e.g., one, two, three, four, five, six, seven, eight, nine, or ten) histidine(s) in SEQ ID NOs: 231, 232, and 233 substituted with an alanine. In some examples of any of the ABPCs described herein, the first ABD comprises an LCVD comprising a CDR1, a CDR2, and a CDR3 of SEQ ID NO: 234, SEQ ID NO: 235, and SEQ ID NO: 236, respectively, with collectively a total of one or more (e.g., one, two, three, four, five, six, seven, eight, nine, or ten) histidine(s) in SEQ ID NOs: 234, 235, and 236 substituted with an alanine. In some examples of any of the ABPCs described herein, the first ABD includes: an HCVD comprising a CDR1, a CDR2, and a CDR3 of SEQ ID NO: 231, SEQ ID NO: 232, and SEQ ID NO: 233, respectively, with collectively a total of one or more (e.g., one, two, three, four, five, six, seven, eight, nine, or ten) histidine(s) in SEQ ID NOs: 231, 232, and 233 substituted with an alanine; and an LCVD comprising a CDR1, a CDR2, and a CDR3 of SEQ ID NO: 234, SEQ ID NO: 235, and SEQ ID NO: 236, respectively, with collectively a total of one or more (e.g., one, two, three, four, five, six, seven, eight, nine, or ten) histidine(s) in SEQ ID NOs: 234, 235, and 236 substituted with an alanine.

In some examples of any of the ABPCs described herein, the first ABD comprises an HCVD of SEQ ID NO: 229, or one of SEQ ID NOs: 237-268.

In some examples of any of the ABPCs described herein, the first ABD comprises an LCVD having a sequence of SEQ ID NO: 230, or one of 269-297.

In some examples of any of the ABPCs described herein, the first ABD includes an LCVD comprising SEQ ID NO: 230, and an HCVD comprising one of SEQ ID NOs: 237-268.

In some examples of any of the ABPCs described herein, the first ABD includes an LCVD comprising SEQ ID NO: 269, and an HCVD comprising: SEQ ID NO: 229, or one of 237-268. In some examples of any of the ABPCs described herein, the first ABD includes an LCVD comprising SEQ ID NO: 270, and an HCVD comprising: SEQ ID NO: 229, or one of 237-268. In some examples of any of the ABPCs described herein, the first ABD includes an LCVD comprising SEQ ID NO: 271, and an HCVD comprising: SEQ ID NO: 229, or one of 237-268. In some examples of any of the ABPCs described herein, the first ABD includes an LCVD comprising SEQ ID NO: 272, and an HCVD comprising: SEQ ID NO: 229, or one of 237-268. In some examples of any of the ABPCs described herein, the first ABD includes an LCVD comprising SEQ ID NO: 273, and an HCVD comprising: SEQ ID NO: 229, or one of 237-268. In some examples of any of the ABPCs described herein, the first ABD includes an LCVD comprising SEQ ID NO: 274, and an HCVD comprising: SEQ ID NO: 229, or one of 237-268. In some examples of any of the ABPCs described herein, the first ABD includes an LCVD comprising SEQ ID NO: 275, and an HCVD comprising: SEQ ID NO: 229, or one of 237-268. In some examples of any of the ABPCs described herein, the first ABD includes an LCVD comprising SEQ ID NO: 276, and an HCVD comprising: SEQ ID NO: 229, or one of 237-268. In some examples of any of the ABPCs described herein, the first ABD includes an LCVD comprising SEQ ID NO: 277, and an HCVD comprising: SEQ ID NO: 229, or one of 237-268. In some examples of any of the ABPCs described herein, the first ABD includes an LCVD comprising SEQ ID NO: 278, and an HCVD comprising: SEQ ID NO: 229, or one of 237-268. In some examples of any of the ABPCs described herein, the first ABD includes an LCVD comprising SEQ ID NO: 279, and an HCVD comprising: SEQ ID NO: 229, or one of 237-268. In some examples of any of the ABPCs described herein, the first ABD includes an LCVD comprising SEQ ID NO: 280, and an HCVD comprising: SEQ ID NO: 229, or one of 237-268. In some examples of any of the ABPCs described herein, the first ABD includes an LCVD comprising SEQ ID NO: 281, and an HCVD comprising: SEQ ID NO: 229, or one of 237-268. In some examples of any of the ABPCs described herein, the first ABD includes an LCVD comprising SEQ ID NO: 282, and an HCVD comprising: SEQ ID NO: 229, or one of 237-268. In some examples of any of the ABPCs described herein, the first ABD includes an LCVD comprising SEQ ID NO: 283, and an HCVD comprising: SEQ ID NO: 229, or one of 237-268. In some examples of any of the ABPCs described herein, the first ABD includes an LCVD comprising SEQ ID NO: 284, and an HCVD comprising: SEQ ID NO: 229, or one of 237-268. In some examples of any of the ABPCs described herein, the first ABD includes an LCVD comprising SEQ ID NO: 285, and an HCVD comprising: SEQ ID NO: 229, or one of 237-268. In some examples of any of the ABPCs described herein, the first ABD includes an LCVD comprising SEQ ID NO: 286, and an HCVD comprising: SEQ ID NO: 229, or one of 237-268. In some examples of any of the ABPCs described herein, the first ABD includes an LCVD comprising SEQ ID NO: 287, and an HCVD comprising: SEQ ID NO: 229, or one of 237-268. In some examples of any of the ABPCs described herein, the first ABD includes an LCVD comprising SEQ ID NO: 288, and an HCVD comprising: SEQ ID NO: 229, or one of 237-268. In some examples of any of the ABPCs described herein, the first ABD includes an LCVD comprising SEQ ID NO: 289, and an HCVD comprising: SEQ ID NO: 229, or one of 237-268. In some examples of any of the ABPCs described herein, the first ABD includes an LCVD comprising SEQ ID NO: 290, and an HCVD comprising: SEQ ID NO: 229, or one of 237-268. In some examples of any of the ABPCs described herein, the first ABD includes an LCVD comprising SEQ ID NO: 291, and an HCVD comprising: SEQ ID NO: 229, or one of 237-268. In some examples of any of the ABPCs described herein, the first ABD includes an LCVD comprising SEQ ID NO: 292, and an HCVD comprising: SEQ ID NO: 229, or one of 237-268. In some examples of any of the ABPCs described herein, the first ABD includes an LCVD comprising SEQ ID NO: 293, and an HCVD comprising: SEQ ID NO: 229, or one of 237-268. In some examples of any of the ABPCs described herein, the first ABD includes an LCVD comprising SEQ ID NO: 294, and an HCVD comprising: SEQ ID NO: 229, or one of 237-268. In some examples of any of the ABPCs described herein, the first ABD includes an LCVD comprising SEQ ID NO: 295, and an HCVD comprising: SEQ ID NO: 229, or one of 237-268. In some examples of any of the ABPCs described herein, the first ABD includes an LCVD comprising SEQ ID NO: 296, and an HCVD comprising: SEQ ID NO: 229, or one of 237-268. In some examples of any of the ABPCs described herein, the first ABD includes an LCVD comprising SEQ ID NO: 297, and an HCVD comprising: SEQ ID NO: 229, or one of 237-268.

In some examples, the first ABD includes an HCVD of MYT9345, optionally with one or more amino acids substituted with a histidine. In some examples, the first ABD includes an LCVD of MYT9345 optionally with one or more amino acids substituted with a histidine. In some examples, the first ABD includes an HCVD of MYT9345 optionally with one or more amino acids optionally substituted with a histidine; and an LCVD of MYT9345 with one or more amino acids substituted with a histidine. In some examples, the HCVD of MYT9345 comprises SEQ ID NO: 301. In some examples, the LCVD of MYT9345 comprises SEQ ID NO: 302.

In some examples, the first ABD comprises an HCVD comprising a CDR1, a CDR2, and a CDR3 of SEQ ID NOs: 303, 304, and 305, respectively, optionally with collectively a total of one or more amino acid positions in SEQ ID NOs: 303, 304, and 305 substituted with a histidine. In some examples, the first ABD comprises an LCVD comprising a CDR1, a CDR2, and a CDR3 of SEQ ID NOs: 306, 307, and 308, respectively, optionally with collectively a total of one or more amino acid positions in SEQ ID NOs: 306, 307, and 308 substituted with a histidine. In some examples, the first ABD includes: an HCVD comprising a CDR1, a CDR2, and a CDR3 of SEQ ID NOs: 303, 304, and 305, respectively, optionally with collectively a total of one or more amino acid positions in NOs: 303, 304, and 305 substituted with a histidine; and an LCVD comprising a CDR1, a CDR2, and a CDR3 of SEQ ID NOs: 306, 307, and 308, respectively, optionally with collectively a total of one or more amino acid positions in SEQ ID NOs: 306, 307, and 308 substituted with a histidine.

In some examples, the first ABD includes an HCVD that is at least 90% identical to SEQ ID NO: 301, where the HCVD optionally includes a histidine at one or more amino acid positions in SEQ ID NO: 301 selected from: 29, 30, 32, 50-54, 58, 60, 96, and 101. In some examples, the first ABD includes an LCVD that is at least 90% identical to SEQ ID NO: 302, where the LCVD optionally includes a histidine at one or more amino acid positions in SEQ ID NO: 302 selected from: 25, 30-33, 35, 55-57, 91, 94-96, and 98. In some examples, the first ABD includes an HCVD that is at least 90% identical to SEQ ID NO: 301, where the HCVD optionally includes a histidine at one or more amino acid positions in SEQ ID NO: 301 selected from: 29, 30, 32, 50-54, 58, 60, 96, and 101, and an LCVD that is at least 90% identical to SEQ ID NO: 302, where the LCVD optionally includes a histidine at one or more amino acid positions in SEQ ID NO: 302 selected from: 25, 30-33, 35, 55-57, 91, 94-96, and 98.

In some examples, an HCVD includes an HCVD that is at least 90% identical to SEQ ID NO: 301, where the HCVD includes a histidine at any of the specific combinations of two or more amino acid positions in SEQ ID NO: 301 listed in Table 7.

In some examples, the first ABD includes an HCVD that is ≥90% (e.g., ≥92%, ≥94%, ≥96%, ≥98%, ≥99%, or 100%) identical to SEQ ID NO: 301, where the HCVD includes an alanine at position 52 in SEQ ID NO: 301.

In some examples the first ABD includes: an HCVD that is ≥90% (e.g., ≥92%, ≥94%, ≥96%, ≥98%, ≥99%, or 100%) identical to SEQ ID NO: 301, where the HCVD includes an alanine at position 52 in SEQ ID NO: 301, and an LCVD that is ≥90% (e.g., ≥92%, ≥94%, ≥96%, ≥98%, ≥99%, or 100%) identical to SEQ ID NO: 302, where the LCVD optionally includes a histidine at one or more amino acid positions in SEQ ID NO: 302 selected from: 25, 30-33, 35, 55-57, 91, 94-96, and 98.

TABLE 7
Exemplary Combinations of Amino Acid Positions in SEQ ID NO: 301
that can be Substituted with Histidine (or Alanine if indicated)

| Col | 1 | 2 | 3 | 4 | 5 | 6 | 7 | 8 | 9 | 10 | 11 | 12 |
|---|---|---|---|---|---|---|---|---|---|---|---|---|
| Pos | 29 | 30 | 32 | 50 | 51 | 52 | 53 | 54 | 58 | 60 | 96 | 101 |

Exemplary 2, 3, and 4 AA position combinations that can be substituted with histidine (or an
alanine at position 52 of SEQ ID NO: 301)

| | |
|---|---|
| A | Each and every unique 2-position combination of X & Y, wherein X and Y are each |
| | selected (without replacement) from a SEQ ID NO: 301 AA position indicated in |
| | columns 1-12. For example, the combinations of X & Y embodied by SEQ ID NO: |
| | 301 AA position 29, 30, 32, 50-54, 58, 60, 96, and 101 (X) + any one of positions 29, |
| | 30, 32, 50-54, 58, 60, 96, and 101 not selected for X (Y). |
| B | Each and every unique 3-position combination of X & Y & Z, wherein X, Y, and Z |
| | are each selected (without replacement) from a SEQ ID NO: 301 AA position |
| | indicated in columns 1-12. For example, the combinations of X & Y & Z embodied by |
| | SEQ ID NO: 301 AA position 29, 30, 32, 50-54, 58, 60, 96, and 101 (X) + any one of |
| | positions 29, 30, 32, 50-54, 58, 60, 96, and 101 not selected for X (Y) + any one of |
| | positions 29, 30, 32, 50-54, 58, 60, 96, and 101 not selected for X or Y (Z). |
| C | Each and every unique 4-position combination of X & Y & Z & α, wherein X, Y, Z, |
| | and α are each selected (without replacement) from a SEQ ID NO: 301 AA position |
| | indicated in columns 1-12. For example, the combinations of X & Y & Z & α |
| | embodied by SEQ ID NO: 301 AA position 29, 30, 32, 50-54, 58, 60, 96, and 101 (X) + |
| | any one of positions 29, 30, 32, 50-54, 58, 60, 96, and 101 not selected for X (Y) + |
| | any one of positions 29, 30, 32, 50-54, 58, 60, 96, and 101 not selected for X or Y (Z) + |
| | any one of positions 29, 30, 32, 50-54, 58, 60, 96, and 101 not selected for X, Y, or |
| | Z (α). |

In some examples, the first ABD includes an LCVD that is ≥90% identical to SEQ ID NO: 302, where the LCVD includes a histidine at any of the specific combinations of two or more amino acid positions in SEQ ID NO: 302 listed in Table 8 and/or where the LCVD includes an alanine at position 95 of SEQ ID NO: 302.

In some examples, the first ABD includes an LCVD that is 90% identical to SEQ ID NO: 302, where the LCVD includes an alanine at position 95 in SEQ ID NO: 302.

In some examples the first ABD includes: an LCVD that is 90% identical to SEQ ID NO: 302, where the LCVD includes an alanine at position 95 in SEQ ID NO: 302, and an HCVD that is ≥90% identical to SEQ ID NO: 301, where the HCVD optionally includes a histidine at one or more amino acid positions in SEQ ID NO: 301 selected from: 29, 30, 32, 50-54, 58, 60, 96, and 101.

In some examples, the first ABD includes an LCVD that is ≥90% identical to SEQ ID NO: 302, where the LCVD optionally includes a histidine at one or more amino acid positions in SEQ ID NO: 302 selected from: 25, 30-33, 35, 55-57, 91, 94-96, and 98 and/or where the LCVD includes an alanine at position 95 in SEQ ID NO: 302. In some examples the first ABD includes: an LCVD that is ≥90% identical to SEQ ID NO: 302, where the LCVD optionally includes a histidine at one or more amino acid positions in SEQ ID NO: 302 selected from: 25, 30-33, 35, 55-57, 91, 94-96, and 98 and/or where the LCVD includes an alanine at position 95 in SEQ ID NO: 302, and an HCVD that is ≥90% identical to SEQ ID NO: 301, where the HCVD optionally includes a histidine at one or more amino acid positions in SEQ ID NO: 301 selected from: 29, 30, 32, 50-54, 58, 60, 96, and 101.

In some examples the first ABD includes an LCVD that is ≥90% identical to SEQ ID NO: 302, where the LCVD optionally includes a histidine at one or more amino acid positions in SEQ ID NO: 302 selected from: 25, 30-33, 35, 55-57, 91, 94-96, and 98 and/or where the LCVD includes an alanine at position 95 in SEQ ID NO: 302, and an HCVD that is ≥90% identical to SEQ ID NO: 301, where the HCVD optionally includes a histidine at one or more amino acid positions in SEQ ID NO: 301 selected from: 29, 30, 32, 50-54, 58, 60, 96, and 101 and/or where the HCVD includes an alanine at position 52.

TABLE 8
Exemplary Combinations of Amino Acid Positions in SEQ ID NO: 302
that can be Substituted with Histidine (or Alanine if indicated)

| Col | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| Pos | 25 | 30 | 31 | 32 | 33 | 35 | 55 | 56 | 57 | 91 | 94 | 95 | 96 | 98 |

Exemplary 2, 3, and 4 AA position combinations that can be substituted with histidine (or an
alanine at position 95 of SEQ ID NO: 302)

| | |
|---|---|
| A | Each and every unique 2-position combination of X & Y, wherein X and Y are each |
| | selected (without replacement) from a SEQ ID NO: 302 AA position indicated in |
| | columns 1-14. For example, the combinations of X & Y embodied by SEQ ID NO: |
| | 302 AA position 25, 30-33, 35, 55-57, 91, 94-96, and 98 (X) + any one of positions 25, |
| | 30-33, 35, 55-57, 91, 94-96, and 98 not selected for X (Y). |
| B | Each and every unique 3-position combination of X & Y & Z, wherein X, Y, and Z are |
| | each selected (without replacement) from a SEQ ID NO: 302 AA position indicated in |
| | columns 1-14. For example, the combinations of X & Y & Z embodied by SEQ ID |
| | NO: 302 AA position 25, 30-33, 35, 55-57, 91, 94-96, and 98 (X) + any one of |
| | positions 25, 30-33, 35, 55-57, 91, 94-96, and 98 not selected for X (Y) + any one of |
| | positions 25, 30-33, 35, 55-57, 91, 94-96, and 98 not selected for X or Y (Z). |
| C | Each and every unique 4-position combination of X & Y & Z & α, wherein X, Y, Z, |
| | and α are each selected (without replacement) from a SEQ ID NO: 302 AA position |
| | indicated in columns 1-14. For example, the combinations of X & Y & Z & α |
| | embodied by SEQ ID NO: 302 AA position 25, 30-33, 35, 55-57, 91, 94-96, and 98 |
| | (X) + any one of positions 25, 30-33, 35, 55-57, 91, 94-96, and 98 not selected for X |
| | (Y) + any one of positions 25, 30-33, 35, 55-57, 91, 94-96, and 98 not selected for X |
| | or Y (Z) + any one of positions 25, 30-33, 35, 55-57, 91, 94-96, and 98 not selected for |
| | X, Y, or Z (α). |

In some examples, the first ABD includes an HCVD that is ≥90% identical to SEQ ID NO: 301, where the HCVD includes a histidine at any of the specific combinations of two or more amino acid positions in SEQ ID NO: 301 listed in Table 7; and an LCVD that that is ≥90% identical to SEQ TD NO: 302, where the LCVD includes a histidine at any of the specific combinations of two or more amino acid positions in SEQ ID NO: 302 listed in Table 8.

In some examples, the first ABD includes an LCVD that is ≥90% identical to SEQ ID NO: 302, where the LCVD includes a histidine at any of the specific combinations of two or more amino acid positions in SEQ ID NO: 302 listed in Table 8, and/or where the LCVD includes an alanine at position 95 in SEQ ID NO: 302; and an HCVD that that is ≥90% identical to SEQ ID NO: 301, where the HCVD includes a histidine at any of the specific combinations of two or more amino acid positions in SEQ ID NO: 301 listed in Table 7. In some examples, the first ABD comprises an LCVD comprising SEQ ID NO: 302, and an HCVD that is ≥90% identical to SEQ ID NO: 301, where the HCVD includes a histidine at any of the specific combinations of two or more amino acid positions in SEQ ID NO: 301 listed in Table 7.

In some examples, the first ABD comprises an LCVD that is ≥90% identical to SEQ ID NO: 302, where the LCVD includes a histidine at position 25 in SEQ ID NO: 302; and an HCVD that is ≥90% identical to SEQ ID NO: 301, where the HCVD includes a histidine at any of the specific combinations of two or more amino acid positions in SEQ ID NO: 301 listed in Table 7.

In some examples, the first ABD comprises an LCVD that is ≥90% identical to SEQ ID NO: 302, where the LCVD includes a histidine at position 30 in SEQ ID NO: 302; and an HCVD that is ≥90% identical to SEQ ID NO: 301, where the HCVD includes a histidine at any of the specific combinations of two or more amino acid positions in SEQ ID NO: 301 listed in Table 7.

In some examples, the first ABD comprises an LCVD that is ≥90% identical to SEQ ID NO: 302, where the LCVD includes a histidine at position 31 in SEQ ID NO: 302; and an HCVD that is ≥90% identical to SEQ ID NO: 301, where the HCVD includes a histidine at any of the specific combinations of two or more amino acid positions in SEQ ID NO: 301 listed in Table 7.

In some examples, the first ABD comprises an LCVD that is ≥90% identical to SEQ ID NO: 302, where the LCVD includes an alanine at position 95 in SEQ ID NO: 302; and an HCVD that is ≥90% identical to SEQ ID NO: 301, wherein the HCVD includes a histidine at any of the specific combinations of two or more amino acid positions in SEQ ID NO: 301 listed in Table 7.

In some examples, the first ABD comprises an LCVD that is ≥90% identical to SEQ ID NO: 302, wherein the LCVD includes a histidine at position 32 in SEQ ID NO: 302; and an HCVD that is ≥90% identical to SEQ ID NO: 301, wherein the HCVD includes a histidine at any of the specific combinations of two or more amino acid positions in SEQ ID NO: 301 listed in Table 7.

In some examples, the first ABD comprises an LCVD that is ≥90% identical to SEQ ID NO: 302, wherein the LCVD includes a histidine at position 33 in SEQ ID NO: 302; and an HCVD that is ≥90% identical to SEQ ID NO: 301, where the HCVD includes a histidine at any of the specific combinations of two or more amino acid positions in SEQ ID NO: 301 listed in Table 7.

In some examples, the first ABD comprises an LCVD that is 90% identical to SEQ ID NO: 302, wherein the LCVD includes a histidine at position 35 in SEQ ID NO: 302; and an HCVD that is ≥90% identical to SEQ ID NO: 301, where the HCVD includes a histidine at any of the specific combinations of two or more amino acid positions in SEQ ID NO: 301 listed in Table 7.

In some examples, the first ABD comprises an LCVD that is ≥90% identical to SEQ ID NO: 302, wherein the LCVD includes a histidine at position 55 in SEQ ID NO: 302; and an HCVD that is ≥90% identical to SEQ ID NO: 301, where the HCVD includes a histidine at any of the specific combinations of two or more amino acid positions in SEQ ID NO: 301 listed in Table 7.

In some examples, the first ABD comprises an LCVD that is ≥90% identical to SEQ ID NO: 302, wherein the LCVD includes a histidine at position 56 in SEQ ID NO: 302; and an HCVD that is ≥90% identical to SEQ ID NO: 301, where the HCVD includes a histidine at any of the specific combinations of two or more amino acid positions in SEQ ID NO: 301 listed in Table 7.

In some examples, the first ABD comprises an LCVD that is ≥90% identical to SEQ ID NO: 302, wherein the LCVD includes a histidine at position 57 in SEQ ID NO: 302; and an HCVD that is ≥90% identical to SEQ ID NO: 301, where the HCVD includes a histidine at any of the specific combinations of two or more amino acid positions in SEQ ID NO: 301 listed in Table 7.

In some examples, the first ABD comprises an LCVD that is ≥90% identical to SEQ ID NO: 302, wherein the LCVD includes a histidine at position 91 in SEQ ID NO: 302; and an HCVD that is ≥90% identical to SEQ ID NO: 301, where the HCVD includes a histidine at any of the specific combinations of two or more amino acid positions in SEQ ID NO: 301 listed in Table 7.

In some examples, the first ABD comprises an LCVD that is ≥90% identical to SEQ ID NO: 302, wherein the LCVD includes a histidine at position 94 in SEQ ID NO: 302; and an HCVD that is ≥90% identical to SEQ ID NO: 301, where the HCVD includes a histidine at any of the specific combinations of two or more amino acid positions in SEQ ID NO: 301 listed in Table 7.

In some examples, the first ABD comprises an LCVD that is ≥90% identical to SEQ ID NO: 302, wherein the LCVD includes a histidine at position 95 in SEQ ID NO: 302; and an HCVD that is ≥90% identical to SEQ ID NO: 301, where the HCVD includes a histidine at any of the specific combinations of two or more amino acid positions in SEQ ID NO: 301 listed in Table 7.

In some examples, the first ABD comprises an LCVD that is ≥90% identical to SEQ ID NO: 302, wherein the LCVD includes a histidine at position 96 in SEQ ID NO: 302; and an HCVD that is ≥90% identical to SEQ ID NO: 301, where the HCVD includes a histidine at any of the specific combinations of two or more amino acid positions in SEQ ID NO: 301 listed in Table 7.

In some examples, the first ABD comprises an LCVD that is ≥90% identical to SEQ ID NO: 302, wherein the LCVD includes a histidine at position 98 in SEQ ID NO: 302; and an HCVD that is ≥90% identical to SEQ ID NO: 301, where the HCVD includes a histidine at any of the specific combinations of two or more amino acid positions in SEQ ID NO: 301 listed in Table 7.

In some examples, the first ABD comprises an HCVD that is ≥90% identical to SEQ ID NO: 301, wherein the HCVD includes a histidine at any one of positions 29, 30, 32, 50-54, 58, 60, 96, and 101 in SEQ ID NO: 301; and an LCVD that is ≥90% identical to SEQ ID NO: 302, where the LCVD includes a histidine at any of the specific combinations of two or more amino acid positions in SEQ ID NO: 302 listed in Table 8.

In some examples, the first ABD comprises an HCVD of SEQ ID NO: 301, or one of 309-342; and/or the first ABD includes an LCVD of SEQ ID NO: 302, or one of 343-374. In some examples of the ABPCs, the first ABD includes an LCVD comprising any one of SEQ ID NO: 302, or one of 343-374 and an HCVD comprising any one of SEQ ID NO: 301, or one of 309-342.

In some examples, the first ABD includes an LCVD comprising SEQ ID NO: 343, and an HCVD comprising one of: SEQ ID NO: 301, or one of 309-342. In some examples, the first ABD includes an LCVD comprising SEQ ID NO: 344, and an HCVD comprising one of: SEQ ID NO: 301, or one of 309-342. In some examples, the first ABD includes an LCVD comprising SEQ ID NO: 345, and an HCVD comprising one of: SEQ ID NO: 301, or one of 309-342. In some examples, the first ABD includes an LCVD comprising SEQ ID NO: 346, and an HCVD comprising one of: SEQ ID NO: 301, or one of 309-342. In some examples, the first ABD includes an LCVD comprising SEQ ID NO: 347, and an HCVD comprising one of: SEQ ID NO: 301, or one of 309-342. In some examples, the first ABD includes an LCVD comprising SEQ ID NO: 348, and an HCVD comprising one of: SEQ ID NO: 301, or one of 309-342. In some examples, the first ABD includes an LCVD comprising SEQ ID NO: 349, and an HCVD comprising one of: SEQ ID NO: 301, or one of 309-342. In some examples, the first ABD includes an LCVD comprising SEQ ID NO: 350, and an HCVD comprising one of: SEQ ID NO: 301, or one of 309-342. In some examples, the first ABD includes an LCVD comprising SEQ ID NO: 351, and an HCVD comprising one of: SEQ ID NO: 301, or one of 309-342. In some examples, the first ABD includes an LCVD comprising SEQ ID NO: 352, and an HCVD comprising one of: SEQ ID NO: 301, or one of 309-342. In some examples, the first ABD includes an LCVD comprising SEQ ID NO: 353, and an HCVD comprising one of: SEQ ID NO: 301, or one of 309-342. In some examples, the first ABD includes an LCVD comprising SEQ ID NO: 354, and an HCVD comprising one of: SEQ ID NO: 301, or one of 309-342. In some examples, the first ABD includes an LCVD comprising SEQ ID NO: 355, and an HCVD comprising one of: SEQ ID NO: 301, or one of 309-342. In some examples, the first ABD includes an LCVD comprising SEQ ID NO: 356, and an HCVD comprising one of: SEQ ID NO: 301, or one of 309-342. In some examples, the first ABD includes an LCVD comprising SEQ ID NO: 357, and an HCVD comprising one of: SEQ ID NO: 301, or one of 309-342. In some examples, the first ABD includes an LCVD comprising SEQ ID NO: 358, and an HCVD comprising one of: SEQ ID NO: 301, or one of 309-342. In some examples, the first ABD includes an LCVD comprising SEQ ID NO: 359, and an HCVD comprising one of: SEQ ID NO: 301, or one of 309-342. In some examples, the first ABD includes an LCVD comprising SEQ ID NO: 360, and an HCVD comprising one of: SEQ ID NO: 301, or one of 309-342. In some examples, the first ABD includes an LCVD comprising SEQ ID NO: 361, and an HCVD comprising one of: SEQ ID NO: 301, or one of 309-342. In some examples, the first ABD includes an LCVD comprising SEQ ID NO: 362, and an HCVD comprising one of: SEQ ID NO: 301, or one of 309-342. In some examples, the first ABD includes an LCVD comprising SEQ ID NO: 363, and an HCVD comprising one of: SEQ ID NO: 301, or one of 309-342. In some examples, the first ABD includes an LCVD comprising SEQ ID NO: 364, and an HCVD comprising one of: SEQ ID NO: 301, or one of 309-342. In some examples, the first ABD includes an LCVD comprising SEQ TD NO: 365, and an HCVD comprising one of: SEQ ID NO: 301, or one of 309-342. In some examples, the first ABD includes an LCVD comprising SEQ ID NO: 366, and an HCVD comprising one of: SEQ ID NO: 301, or one of 309-342. In some examples, the first ABD includes an LCVD comprising SEQ ID NO: 367, and an HCVD comprising one of: SEQ ID NO: 301, or one of 309-342. In some examples, the first ABD includes an LCVD comprising SEQ ID NO: 368, and an HCVD comprising one of: SEQ ID NO: 301, or one of 309-342. In some examples, the first ABD includes an LCVD comprising SEQ ID NO: 369, and an HCVD comprising one of: SEQ ID NO: 301, or one of 309-342. In some examples, the first ABD includes an LCVD comprising SEQ ID NO: 370, and an HCVD comprising one of: SEQ ID NO: 301, or one of 309-342. In some examples, the first ABD includes an LCVD comprising SEQ ID NO: 371, and an HCVD comprising one of: SEQ ID NO: 301, or one of 309-342. In some examples, the first ABD includes an LCVD comprising SEQ ID NO: 372, and an HCVD comprising one of: SEQ ID NO: 301, or one of 309-342. In some examples, the first ABD includes an LCVD comprising SEQ ID NO: 373, and an HCVD comprising one of: SEQ ID NO: 301, or one of 309-342. In some examples, the first ABD includes an LCVD comprising SEQ ID NO: 374, and an HCVD comprising one of: SEQ ID NO: 301, or one of 309-342.

In some examples, the first ABD includes an HCVD of MYT9359 with one or more amino acids substituted with a histidine. In some examples, the first ABD includes an LCVD of MYT9359 with one or more amino acids substituted with a histidine. In some examples, the first ABD includes an HCVD of MYT9359 with one or more amino acids optionally substituted with a histidine; and an LCVD of MYT9359 with one or more amino acids substituted with a histidine. In some examples, the HCVD of MYT9359 comprises SEQ ID NO: 375. In some examples, the LCVD of MYT9359 comprises SEQ ID NO: 376.

In some examples, the first ABD comprises an HCVD comprising a CDR1, a CDR2, and a CDR3 of SEQ ID NOs: 377, 378, and 379, respectively, optionally with collectively a total of one or more amino acid positions in SEQ ID NOs: 377, 378, and 379 substituted with a histidine. In some examples, the first ABD comprises an LCVD comprising a CDR1, a CDR2, and a CDR3 of SEQ ID NOs: 380, 381, and 382, respectively, optionally with collectively a total of one or more amino acid positions in SEQ ID NOs: 380, 381, and 382 substituted with a histidine. In some examples, the first ABD includes: an HCVD comprising a CDR1, a CDR2, and a CDR3 of SEQ ID NOs: 377, 378, and 379, respectively, optionally with collectively a total of one or more amino acid positions in NOs: 377, 378, and 379 optionally substituted with a histidine; and an LCVD comprising a CDR1, a CDR2, and a CDR3 of SEQ ID NOs: 380, 381, and 382, respectively, optionally with collectively a total of one or more amino acid positions in SEQ ID NOs: 380, 381, and 382 substituted with a histidine.

In some examples, the first ABD includes an HCVD that is at least 90% identical to SEQ ID NO: 375, where the HCVD optionally includes a histidine at one or more amino acid positions in SEQ ID NO: 375 selected from: 26, 27, 30, 32, 34, 51, 52, 55, 58, 59, 63, 97, 99, and 108. In some examples, the first ABD includes an LCVD that is at least 90% identical to SEQ ID NO: 376, where the LCVD optionally includes a histidine at one or more amino acid positions in SEQ ID NO: 376 selected from: 25, 29, 30, 31, 32, 33, 50, 51, 52, 53, 89, 91, 92, 93, 96, 97, 100 and 102. In some examples, the first ABD includes an HCVD that is at least 90% identical to SEQ ID NO: 375, where the HCVD optionally includes a histidine at one or more amino acid positions in SEQ ID NO: 375 selected from: 26, 27, 30, 32, 34, 51, 52, 55, 58, 59, 63, 97, 99, and 108, and an LCVD that is at least 90% identical to SEQ ID NO: 376, where the LCVD optionally includes a histidine at one or more amino acid positions in SEQ ID NO: 376 selected from: 25, 29, 30, 31, 32, 33, 50, 51, 52, 53, 89, 91, 92, 93, 96, 97, 100 and 102.

In some examples, an HCVD includes an HCVD that is at least 90% identical to SEQ ID NO: 375, where the HCVD includes a histidine at any of the specific combinations of two or more amino acid positions in SEQ ID NO: 375 listed in Table 9.

In some examples, the first ABD includes an HCVD that is ≥90% (e.g., ≥92%, ≥94%, ≥96%, ≥98%, ≥99%, or 100%) identical to SEQ ID NO: 375, where the HCVD includes an alanine at position 107 in SEQ ID NO: 375.

In some examples the first ABD includes: an HCVD that is ≥90% (e.g., ≥92%, ≥94%, ≥96%, ≥98%, ≥99%, or 100%) identical to SEQ ID NO: 375, where the HCVD includes an alanine at position 107 in SEQ ID NO: 375, and an LCVD that is ≥90% (e.g., ≥92%, ≥94%, ≥96%, ≥98%, ≥99%, or 100%) identical to SEQ ID NO: 376, where the LCVD optionally includes a histidine at one or more amino acid positions in SEQ ID NO: 376 selected from: 26, 27, 30, 32, 34, 51, 52, 55, 58, 59, 63, 97, 99, and 108.

TABLE 9
Exemplary Combinations of Amino Acid Positions in SEQ ID NO: 375
that can be Substituted with Histidine (or Alanine if indicated)

| Col | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| Pos | 26 | 27 | 30 | 32 | 34 | 51 | 52 | 55 | 58 | 59 | 63 | 97 | 99 | 108 |

Exemplary 2, 3, and 4 AA position combinations that can be substituted with histidine (or an
alanine at position 107 of SEQ ID NO: 375)

| | |
|---|---|
| A | Each and every unique 2-position combination of X & Y, wherein X and Y are each |
| | selected (without replacement) from a SEQ ID NO: 375 AA position indicated in |
| | columns 1-14. For example, the combinations of X & Y embodied by SEQ ID NO: |
| | 375 AA position 26, 27, 30, 32, 34, 51, 52, 55, 58, 59, 63, 97, 99, and 108 (X) + any |
| | one of positions 26, 27, 30, 32, 34, 51, 52, 55, 58, 59, 63, 97, 99, and 108 not selected |
| | for X (Y). |
| B | Each and every unique 3-position combination of X & Y & Z, wherein X, Y, and Z are |
| | each selected (without replacement) from a SEQ ID NO: 375 AA position indicated in |
| | columns 1-14. For example, the combinations of X & Y & Z embodied by SEQ ID |
| | NO: 375 AA position 26, 27, 30, 32, 34, 51, 52, 55, 58, 59, 63, 97, 99, and 108 (X) + |
| | any one of positions 26, 27, 30, 32, 34, 51, 52, 55, 58, 59, 63, 97, 99, and 108 not |
| | selected for X (Y) + any one of positions 26, 27, 30, 32, 34, 51, 52, 55, 58, 59, 63, 97, |
| | 99, and 108 not selected for X or Y (Z). |
| C | Each and every unique 4-position combination of X & Y & Z & α, wherein X, Y, Z, |
| | and α are each selected (without replacement) from a SEQ ID NO: 375 AA position |
| | indicated in columns 1-14. For example, the combinations of X & Y & Z & α |
| | embodied by SEQ ID NO: 375 AA position 26, 27, 30, 32, 34, 51, 52, 55, 58, 59, 63, |
| | 97, 99, and 108 (X) + any one of positions 26, 27, 30, 32, 34, 51, 52, 55, 58, 59, 63, |
| | 97, 99, and 108 not selected for X (Y) + any one of positions 26, 27, 30, 32, 34, 51, 52, |
| | 55, 58, 59, 63, 97, 99, and 108 not selected for X or Y (Z) + any one of positions 26, |
| | 27, 30, 32, 34, 51, 52, 55, 58, 59, 63, 97, 99, and 108 not selected for X, Y, or Z (α). |

In some examples, the first ABD includes an LCVD that is ≥90% identical to SEQ ID NO: 376, where the LCVD includes a histidine at any of the specific combinations of two or more amino acid positions in SEQ ID NO: 376 listed in Table 10 and/or where the LCVD includes an alanine at position 98 of SEQ ID NO: 376.

In some examples, the first ABD includes an LCVD that is ≥90% identical to SEQ ID NO: 376, where the LCVD includes an alanine at position 98 in SEQ ID NO: 376.

In some examples the first ABD includes: an LCVD that is 90% identical to SEQ ID NO: 376, where the LCVD includes an alanine at position 98 in SEQ ID NO: 376, and an HCVD that is ≥90% identical to SEQ ID NO: 375, where the HCVD optionally includes a histidine at one or more amino acid positions in SEQ ID NO: 375 selected from: 26, 27, 30, 32, 34, 51, 52, 55, 58, 59, 63, 97, 99, and 108.

In some examples, the first ABD includes an LCVD that is 90% identical to SEQ ID NO: 376, where the LCVD optionally includes a histidine at one or more amino acid positions in SEQ ID NO: 376 selected from: 25, 29, 30, 31, 32, 33, 50, 51, 52, 53, 89, 91, 92, 93, 96, 97, 100 and 102 and/or where the LCVD includes an alanine at position 98 in SEQ ID NO: 376. In some examples the first ABD includes: an LCVD that is ≥90% identical to SEQ ID NO: 376, where the LCVD optionally includes a histidine at one or more amino acid positions in SEQ ID NO: 376 selected from: 25, 29, 30, 31, 32, 33, 50, 51, 52, 53, 89, 91, 92, 93, 96, 97, 100 and 102 and/or where the LCVD includes an alanine at position 98 in SEQ ID NO: 376, and an HCVD that is 90% identical to SEQ ID NO: 375, where the HCVD optionally includes a histidine at one or more amino acid positions in SEQ ID NO: 375 selected from: 26, 27, 30, 32, 34, 51, 52, 55, 58, 59, 63, 97, 99, and 108.

In some examples the first ABD includes an LCVD that is ≥90% identical to SEQ ID NO: 376, where the LCVD optionally includes a histidine at one or more amino acid positions in SEQ ID NO: 376 selected from: 25, 29, 30, 31, 32, 33, 50, 51, 52, 53, 89, 91, 92, 93, 96, 97, 100 and 102 and/or where the LCVD includes an alanine at position 98 in SEQ ID NO: 376, and an HCVD that is ≥90% identical to SEQ ID NO: 375, where the HCVD optionally includes a histidine at one or more amino acid positions in SEQ ID NO: 375 selected from: 26, 27, 30, 32, 34, 51, 52, 55, 58, 59, 63, 97, 99, and 108 and/or where the HCVD includes an alanine at position 107.

TABLE 10
Exemplary Combinations of Amino Acid Positions in SEQ ID NO: 376
that can be Substituted with Histidine (or Alanine if indicated)

| Col | 1 | 2 | 3 | 4 | 5 | 6 | 7 | 8 | 9 | 10 | 11 | 12 | 13 | 14 | 15 | 16 | 17 | 18 |
|---|---|---|---|---|---|---|---|---|---|---|---|---|---|---|---|---|---|---|
| Pos | 25 | 29 | 30 | 31 | 32 | 33 | 50 | 51 | 52 | 53 | 89 | 91 | 92 | 93 | 96 | 97 | 100 | 102 |

Exemplary 2, 3, and 4 AA position combinations that can be substituted with histidine (or an
alanine at position 98 of SEQ ID NO: 376)

| | |
|---|---|
| A | Each and every unique 2-position combination of X & Y, wherein X and Y are each |
| | selected (without replacement) from a SEQ ID NO: 376 AA position indicated in |
| | columns 1-18. For example, the combinations of X & Y embodied by SEQ ID NO: 376 |
| | AA position 25, 29, 30, 31, 32, 33, 50, 51, 52, 53, 89, 91, 92, 93, 96, 97, 100 and 102 |
| | (X) + any one of positions 25, 29, 30, 31, 32, 33, 50, 51, 52, 53, 89, 91, 92, 93, 96, 97, |
| | 100 and 102 not selected for X (Y). |
| B | Each and every unique 3-position combination of X & Y & Z, wherein X, Y, and Z are |
| | each selected (without replacement) from a SEQ ID NO: 376 AA position indicated in |
| | columns 1-18. For example, the combinations of X & Y & Z embodied by SEQ ID |
| | NO: 376 AA position 25, 29, 30, 31, 32, 33, 50, 51, 52, 53, 89, 91, 92, 93, 96, 97, 100 |
| | and 102 (X) + any one of positions 25, 29, 30, 31, 32, 33, 50, 51, 52, 53, 89, 91, 92, 93, |
| | 96, 97, 100 and 102 not selected for X (Y) + any one of positions 25, 29, 30, 31, 32, |
| | 33, 50, 51, 52, 53, 89, 91, 92, 93, 96, 97, 100 and 102 not selected for X or Y (Z). |
| C | Each and every unique 4-position combination of X & Y & Z & α, wherein X, Y, Z, |
| | and α are each selected (without replacement) from a SEQ ID NO: 376 AA position |
| | indicated in columns 1-18. For example, the combinations of X & Y & Z & α |
| | embodied by SEQ ID NO: 376 AA position 25, 29, 30, 31, 32, 33, 50, 51, 52, 53, 89, |
| | 91, 92, 93, 96, 97, 100 and 102 (X) + any one of positions 25, 29, 30, 31, 32, 33, 50, |
| | 51, 52, 53, 89, 91, 92, 93, 96, 97, 100 and 102 not selected for X (Y) + any one of |
| | positions 25, 29, 30, 31, 32, 33, 50, 51, 52, 53, 89, 91, 92, 93, 96, 97, 100 and 102 not |
| | selected for X or Y (Z) + any one of positions 25, 29, 30, 31, 32, 33, 50, 51, 52, 53, 89, |
| | 91, 92, 93, 96, 97, 100 and 102 not selected for X, Y, or Z (α). |

In some examples, the first ABD includes an HCVD that is ≥90% identical to SEQ ID NO: 375, where the HCVD includes a histidine at any of the specific combinations of two or more amino acid positions in SEQ ID NO: 375 listed in Table 9; and an LCVD that that is ≥90% identical to SEQ ID NO: 376, where the LCVD includes a histidine at any of the specific combinations of two or more amino acid positions in SEQ ID NO: 376 listed in Table 10.

In some examples, the first ABD includes an LCVD that is ≥90% identical to SEQ ID NO: 376, where the LCVD includes a histidine at any of the specific combinations of two or more amino acid positions in SEQ ID NO: 376 listed in Table 10, and/or where the LCVD includes an alanine at position 98 in SEQ ID NO: 376; and an HCVD that that is ≥90% identical to SEQ ID NO: 375, where the HCVD includes a histidine at any of the specific combinations of two or more amino acid positions in SEQ ID NO: 375 listed in Table 9.

In some examples, the first ABD comprises an LCVD comprising SEQ ID NO: 376, and an HCVD that is ≥90% identical to SEQ ID NO: 375, where the HCVD includes a histidine at any of the specific combinations of two or more amino acid positions in SEQ ID NO: 375 listed in Table 9.

In some examples, the first ABD comprises an LCVD that is ≥90% identical to SEQ ID NO: 376, where the LCVD includes a histidine at position 25 in SEQ ID NO: 376; and an HCVD that is ≥90% identical to SEQ ID NO: 375, where the HCVD includes a histidine at any of the specific combinations of two or more amino acid positions in SEQ ID NO: 375 listed in Table 9.

In some examples, the first ABD comprises an LCVD that is ≥90% identical to SEQ ID NO: 376, where the LCVD includes a histidine at position 29 in SEQ ID NO: 376; and an HCVD that is ≥90% identical to SEQ ID NO: 375, where the HCVD includes a histidine at any of the specific combinations of two or more amino acid positions in SEQ ID NO: 375 listed in Table 9.

In some examples, the first ABD comprises an LCVD that is ≥90% identical to SEQ ID NO: 376, where the LCVD includes a histidine at position 30 in SEQ ID NO: 376; and an HCVD that is at least 90% identical to SEQ ID NO: 375, where the HCVD includes a histidine at any of the specific combinations of two or more amino acid positions in SEQ ID NO: 375 listed in Table 9.

In some examples, the first ABD comprises an LCVD that is ≥90% identical to SEQ ID NO: 376, where the LCVD includes an alanine at position 98 in SEQ ID NO: 376; and an HCVD that is at least 90% identical to SEQ ID NO: 375, wherein the HCVD includes a histidine at any of the specific combinations of two or more amino acid positions in SEQ ID NO: 375 listed in Table 9.

In some examples, the first ABD comprises an LCVD that is ≥90% identical to SEQ ID NO: 376, wherein the LCVD includes a histidine at position 31 in SEQ ID NO: 376; and an HCVD that is at least 90% identical to SEQ ID NO: 375, wherein the HCVD includes a histidine at any of the specific combinations of two or more amino acid positions in SEQ ID NO: 375 listed in Table 9.

In some examples, the first ABD comprises an LCVD that is ≥90% identical to SEQ ID NO: 376, wherein the LCVD includes a histidine at position 33 in SEQ ID NO: 376; and an HCVD that is at least 90% identical to SEQ ID NO: 375, where the HCVD includes a histidine at any of the specific combinations of two or more amino acid positions in SEQ ID NO: 375 listed in Table 9.

In some examples, the first ABD comprises an LCVD that is ≥90% identical to SEQ ID NO: 376, wherein the LCVD includes a histidine at position 50 in SEQ ID NO: 376; and an HCVD that is at least 90% identical to SEQ ID NO: 375, where the HCVD includes a histidine at any of the specific combinations of two or more amino acid positions in SEQ ID NO: 375 listed in Table 9.

In some examples, the first ABD comprises an LCVD that is ≥90% identical to SEQ ID NO: 376, wherein the LCVD includes a histidine at position 51 in SEQ ID NO: 376; and an HCVD that is at least 90% identical to SEQ ID NO: 375, where the HCVD includes a histidine at any of the specific combinations of two or more amino acid positions in SEQ ID NO: 375 listed in Table 9.

In some examples, the first ABD comprises an LCVD that is ≥90% identical to SEQ ID NO: 376, wherein the LCVD includes a histidine at position 52 in SEQ ID NO: 376; and an HCVD that is at least 90% identical to SEQ ID NO: 375, where the HCVD includes a histidine at any of the specific combinations of two or more amino acid positions in SEQ ID NO: 375 listed in Table 9.

In some examples, the first ABD comprises an LCVD that is ≥90% identical to SEQ ID NO: 376, wherein the LCVD includes a histidine at position 53 in SEQ ID NO: 376; and an HCVD that is at least 90% identical to SEQ ID NO: 375, where the HCVD includes a histidine at any of the specific combinations of two or more amino acid positions in SEQ ID NO: 375 listed in Table 9.

In some examples, the first ABD comprises an LCVD that is ≥90% identical to SEQ ID NO: 376, wherein the LCVD includes a histidine at position 89 in SEQ ID NO: 376; and an HCVD that is at least 90% identical to SEQ ID NO: 375, where the HCVD includes a histidine at any of the specific combinations of two or more amino acid positions in SEQ ID NO: 375 listed in Table 9.

In some examples, the first ABD comprises an LCVD that is 90% identical to SEQ ID NO: 376, wherein the LCVD includes a histidine at position 91 in SEQ ID NO: 376; and an HCVD that is at least 90% identical to SEQ ID NO: 375, where the HCVD includes a histidine at any of the specific combinations of two or more amino acid positions in SEQ ID NO: 375 listed in Table 9.

In some examples, the first ABD comprises an LCVD that is 90% identical to SEQ ID NO: 376, wherein the LCVD includes a histidine at position 92 in SEQ ID NO: 376; and an HCVD that is at least 90% identical to SEQ ID NO: 375, where the HCVD includes a histidine at any of the specific combinations of two or more amino acid positions in SEQ ID NO: 375 listed in Table 9.

In some examples, the first ABD comprises an LCVD that is 90% identical to SEQ ID NO: 376, wherein the LCVD includes a histidine at position 93 in SEQ ID NO: 376; and an HCVD that is at least 90% identical to SEQ ID NO: 375, where the HCVD includes a histidine at any of the specific combinations of two or more amino acid positions in SEQ ID NO: 375 listed in Table 9.

In some examples, the first ABD comprises an LCVD that is ≥90% identical to SEQ ID NO: 376, wherein the LCVD includes a histidine at position 96 in SEQ ID NO: 376; and an HCVD that is at least 90% identical to SEQ ID NO: 375, where the HCVD includes a histidine at any of the specific combinations of two or more amino acid positions in SEQ ID NO: 375 listed in Table 9.

In some examples, the first ABD comprises an LCVD that is ≥90% identical to SEQ ID NO: 376, wherein the LCVD includes a histidine at position 97 in SEQ ID NO: 376; and an HCVD that is at least 90% identical to SEQ ID NO: 375, where the HCVD includes a histidine at any of the specific combinations of two or more amino acid positions in SEQ ID NO: 375 listed in Table 9.

In some examples, the first ABD comprises an LCVD that is ≥90% identical to SEQ ID NO: 376, wherein the LCVD includes a histidine at position 100 in SEQ ID NO: 376; and an HCVD that is at least 90% identical to SEQ ID NO: 375, where the HCVD includes a histidine at any of the specific combinations of two or more amino acid positions in SEQ ID NO: 375 listed in Table 9.

In some examples, the first ABD comprises an LCVD that is ≥90% identical to SEQ ID NO: 376, wherein the LCVD includes a histidine at position 102 in SEQ ID NO: 376; and an HCVD that is at least 90% identical to SEQ ID NO: 375, where the HCVD includes a histidine at any of the specific combinations of two or more amino acid positions in SEQ ID NO: 375 listed in Table 9.

In some examples, the first ABD comprises an HCVD that is ≥90% identical to SEQ ID NO: 375, wherein the HCVD includes a histidine at any one of positions 26, 27, 30, 32, 34, 51, 52, 55, 58, 59, 63, 97, 99, and 108 in SEQ ID NO: 375; and an LCVD that is ≥90% identical to SEQ ID NO: 376, where the LCVD includes a histidine at any of the specific combinations of two or more amino acid positions in SEQ ID NO: 376 listed in Table 10.

In some examples, the first ABD comprises an HCVD of SEQ ID NO: 375, or one of 383-423; and/or the first ABD includes an LCVD of SEQ ID NO: 376, or one of 424-458. In 30 some examples of the ABPCs, the first ABD includes an LCVD comprising any one of SEQ ID NO: 376, or one of 424-458 and an HCVD comprising any one of SEQ ID NO: 375, or one of 383-423.

In some examples, the first ABD includes an LCVD comprising SEQ ID NO: 424, and an HCVD comprising one of: SEQ ID NO: 375, or one of 383-423. In some examples, the first 5 ABD includes an LCVD comprising SEQ ID NO: 425, and an HCVD comprising one of: SEQ ID NO: 375, or one of 383-423. In some examples, the first ABD includes an LCVD comprising SEQ ID NO: 426, and an HCVD comprising one of: SEQ ID NO: 375, or one of 383-423. In some examples, the first ABD includes an LCVD comprising SEQ ID NO: 427, and an HCVD comprising one of: SEQ ID NO: 375, or one of 383-423. In some examples, the first ABD includes an LCVD comprising SEQ ID NO: 428, and an HCVD comprising one of: SEQ ID NO: 375, or one of 383-423. In some examples, the first ABD includes an LCVD comprising SEQ ID NO: 429, and an HCVD comprising one of: SEQ ID NO: 375, or one of 383-423. In some examples, the first ABD includes an LCVD comprising SEQ ID NO: 430, and an HCVD comprising one of: SEQ ID NO: 375, or one of 383-423. In some examples, the first ABD includes an LCVD comprising SEQ ID NO: 431, and an HCVD comprising one of: SEQ ID NO: 375, or one of 383-423. In some examples, the first ABD includes an LCVD comprising SEQ ID NO: 432, and an HCVD comprising one of: SEQ ID NO: 375, or one of 383-423. In some examples, the first ABD includes an LCVD comprising SEQ ID NO: 433, and an HCVD comprising one of: SEQ ID NO: 375, or one of 383-423. In some examples, the first ABD includes an LCVD comprising SEQ ID NO: 434, and an HCVD comprising one of: SEQ ID NO: 375, or one of 383-423. In some examples, the first ABD includes an LCVD comprising SEQ ID NO: 435, and an HCVD comprising one of: SEQ ID NO: 375, or one of 383-423. In some examples, the first ABD includes an LCVD comprising SEQ ID NO: 436, and an HCVD comprising one of: SEQ ID NO: 375, or one of 383-423. In some examples, the first ABD includes an LCVD comprising SEQ ID NO: 437, and an HCVD comprising one of: SEQ ID NO: 375, or one of 383-423. In some examples, the first ABD includes an LCVD comprising SEQ ID NO: 438, and an HCVD comprising one of: SEQ ID NO: 375, or one of 383-423. In some examples, the first ABD includes an LCVD comprising SEQ ID NO: 439, and an HCVD comprising one of: SEQ ID NO: 375, or one of 383-423. In some examples, the first ABD includes an LCVD comprising SEQ ID NO: 440, and an HCVD comprising one of: SEQ ID NO: 375, or one of 383-423. In some examples, the first ABD includes an LCVD comprising SEQ ID NO: 441, and an HCVD comprising one of: SEQ ID NO: 375, or one of 383-423. In some examples, the first ABD includes an LCVD comprising SEQ ID NO: 442, and an HCVD comprising one of: SEQ ID NO: 375, or one of 383-423. In some examples, the first ABD includes an LCVD comprising SEQ ID NO: 443, and an HCVD comprising one of: SEQ ID NO: 375, or one of 383-423. In some examples, the first ABD includes an LCVD comprising SEQ ID NO: 444, and an HCVD comprising one of: SEQ ID NO: 375, or one of 383-423. In some examples, the first ABD includes an LCVD comprising SEQ ID NO: 445, and an HCVD comprising one of: SEQ ID NO: 375, or one of 383-423. In some examples, the first ABD includes an LCVD comprising SEQ ID NO: 446, and an HCVD comprising one of: SEQ ID NO: 375, or one of 383-423. In some examples, the first ABD includes an LCVD comprising SEQ ID NO: 447, and an HCVD comprising one of: SEQ ID NO: 375, or one of 383-423. In some examples, the first ABD includes an LCVD comprising SEQ ID NO: 448, and an HCVD comprising one of: SEQ ID NO: 375, or one of 383-423. In some examples, the first ABD includes an LCVD comprising SEQ ID NO: 449, and an HCVD comprising one of: SEQ ID NO: 375, or one of 383-423. In some examples, the first ABD includes an LCVD comprising SEQ ID NO: 450, and an HCVD comprising one of: SEQ ID NO: 375, or one of 383-423. In some examples, the first ABD includes an LCVD comprising SEQ ID NO: 451, and an HCVD comprising one of: SEQ ID NO: 375, or one of 383-423. In some examples, the first ABD includes an LCVD comprising SEQ ID NO: 452, and an HCVD comprising one of: SEQ ID NO: 375, or one of 383-423. In some examples, the first ABD includes an LCVD comprising SEQ ID NO: 453, and an HCVD comprising one of: SEQ ID NO: 375, or one of 383-423. In some examples, the first ABD includes an LCVD comprising SEQ ID NO: 454, and an HCVD comprising one of: SEQ ID NO: 375, or one of 383-423. In some examples, the first ABD includes an LCVD comprising SEQ ID NO: 455, and an HCVD comprising one of: SEQ ID NO: 375, or one of 383-423. In some examples, the first ABD includes an LCVD comprising SEQ ID NO: 456, and an HCVD comprising one of: SEQ ID NO: 375, or one of 383-423. In some examples, the first ABD includes an LCVD comprising SEQ ID NO: 457, and an HCVD comprising one of: SEQ ID NO: 375, or one of 383-423. In some examples, the first ABD includes an LCVD comprising SEQ ID NO: 458, and an HCVD comprising one of: SEQ ID NO: 375, or one of 383-423.

In some examples, the first ABD includes an HCVD of MYT9361 with one or more amino acids substituted with a histidine. In some examples, the first ABD includes an LCVD of MYT9361 with one or more amino acids substituted with a histidine. In some examples, the first ABD includes an HCVD of MYT9361 with one or more amino acids optionally substituted with a histidine; and an LCVD of MYT9361 with one or more amino acids substituted with a histidine. In some examples, the HCVD of MYT9361 comprises SEQ ID NO: 459. In some examples, the LCVD of MYT9361 comprises SEQ ID NO: 460.

In some examples, the first ABD comprises an HCVD comprising a CDR1, a CDR2, and a CDR3 of SEQ ID NOs: 461, 462, and 463, respectively, optionally with collectively a total of one or more amino acid positions in SEQ ID NOs: 461, 462, and 463 substituted with a histidine. In some examples, the first ABD comprises an LCVD comprising a CDR1, a CDR2, and a CDR3 of SEQ ID NOs: 464, 465, and 466, respectively, optionally with collectively a total of one or more amino acid positions in SEQ ID NOs: 464, 465, and 466 substituted with a histidine. In some examples, the first ABD includes: an HCVD comprising a CDR1, a CDR2, and a CDR3 of SEQ ID NOs: 461, 462, and 463, respectively, optionally with collectively a total of one or more amino acid positions in NOs: 461, 462, and 463 substituted with a histidine; and an LCVD comprising a CDR1, a CDR2, and a CDR3 of SEQ ID NOs: 464, 465, and 466, respectively, optionally with collectively a total of one or more amino acid positions in SEQ ID NOs: 464, 465, and 466 substituted with a histidine.

In some examples, the first ABD includes an HCVD that is at least 90% identical to SEQ ID NO: 459, where the HCVD optionally includes a histidine at one or more amino acid positions in SEQ ID NO: 459 selected from: 32, 99, 101, 102, 106 and 111. In some examples, the first ABD includes an LCVD that is at least 90% identical to SEQ ID NO: 460, where the LCVD optionally includes a histidine at one or more amino acid positions in SEQ ID NO: 460 selected from: 30, 31, 33, 51, 92 and 95. In some examples, the first ABD includes an HCVD that is at least 90% identical to SEQ ID NO: 459, where the HCVD optionally includes a histidine at one or more amino acid positions in SEQ ID NO: 459 selected from: 32, 99, 101, 102, 106 and 111, and an LCVD that is at least 90% identical to SEQ ID NO: 460, where the LCVD optionally includes a histidine at one or more amino acid positions in SEQ ID NO: 460 selected from: 30, 31, 33, 51, 92 and 95.

In some examples, an HCVD includes an HCVD that is at least 90% identical to SEQ ID NO: 459, where the HCVD includes a histidine at any of the specific combinations of two or more amino acid positions in SEQ ID NO: 459 listed in Table 11.

TABLE 11
Exemplary Combinations of Amino Acid Positions in SEQ
ID NO: 459 that can be Substituted with Histidine

| Col | 1 | 2 | 3 | 4 | 5 | 6 |
|---|---|---|---|---|---|---|
| Pos | 32 | 99 | 101 | 102 | 106 | 111 |

Exemplary 2, 3, and 4 AA position combinations that can be substituted with histidine

| | |
|---|---|
| A | Each and every unique 2-position combination of X & Y, wherein X and Y are each |
| | selected (without replacement) from a SEQ ID NO: 459 AA position indicated in |
| | columns 1-6. For example, the combinations of X & Y embodied by SEQ ID NO: 459 |
| | AA position 32, 99, 101, 102, 106 and 111 (X) + any one of positions 32, 99, 101, |
| | 102, 106 and 111 not selected for X (Y). |
| B | Each and every unique 3-position combination of X & Y & Z, wherein X, Y, and Z |
| | are each selected (without replacement) from a SEQ ID NO: 459 AA position |
| | indicated in columns 1-6. For example, the combinations of X & Y & Z embodied by |
| | SEQ ID NO: 459 AA position 32, 99, 101, 102, 106 and 111 (X) + any one of |
| | positions 32, 99, 101, 102, 106 and 111 not selected for X (Y) + any one of positions |
| | 32, 99, 101, 102, 106 and 111 not selected for X or Y (Z). |
| C | Each and every unique 4-position combination of X & Y & Z & α, wherein X, Y, Z, |
| | and α are each selected (without replacement) from a SEQ ID NO: 459 AA position |
| | indicated in columns 1-6. For example, the combinations of X & Y & Z & α |
| | embodied by SEQ ID NO: 459 AA position 32, 99, 101, 102, 106 and 111 (X) + any |
| | one of positions 32, 99, 101, 102, 106 and 111 not selected for X (Y) + any one of |
| | positions 32, 99, 101, 102, 106 and 111 not selected for X or Y (Z) + any one of |
| | positions 32, 99, 101, 102, 106 and 111 not selected for X, Y, or Z (α). |

In some examples, the first ABD includes an LCVD that is ≥90% identical to SEQ ID NO: 460, where the LCVD includes a histidine at any of the specific combinations of two or more amino acid positions in SEQ ID NO: 460 listed in Table 12.

In some examples, the first ABD includes an LCVD that is ≥90% identical to SEQ ID NO: 460, where the LCVD optionally includes a histidine at one or more amino acid positions in SEQ ID NO: 460 selected from: 30, 31, 33, 51, 92 and 95. In some examples the first ABD includes: an LCVD that is ≥90% identical to SEQ ID NO: 460, where the LCVD optionally includes a histidine at one or more amino acid positions in SEQ ID NO: 460 selected from: 30, 31, 33, 51, 92 and 95, and an HCVD that is ≥90% identical to SEQ ID NO: 459, where the HCVD optionally includes a histidine at one or more amino acid positions in SEQ ID NO: 459 selected from: 32, 99, 101, 102, 106 and 111.

In some examples the first ABD includes an LCVD that is ≥90% identical to SEQ TD NO: 460, where the LCVD optionally includes a histidine at one or more amino acid positions in SEQ ID NO: 460 selected from: 30, 31, 33, 51, 92 and 95, and an HCVD that is ≥90% identical to SEQ ID NO: 459, where the HCVD optionally includes a histidine at one or more amino acid positions in SEQ TD NO: 459 selected from: 32, 99, 101, 102, 106 and 111.

TABLE 12
Exemplary Combinations of Amino Acid Positions in SEQ
ID NO: 460 that can be Substituted with Histidine

| Col | 1 | 2 | 3 | 4 | 5 | 6 |
|---|---|---|---|---|---|---|
| Pos | 30 | 31 | 33 | 51 | 92 | 95 |

| | Exemplary 2, 3, and 4 AA position combinations that can be substituted with histidine |
|---|---|
| A | Each and every unique 2-position combination of X & Y, wherein X and Y are each |
| | selected (without replacement) from a SEQ ID NO: 460 AA position indicated in |
| | columns 1-6. For example, the combinations of X & Y embodied by SEQ ID NO: |
| | 460 AA position 30, 31, 33, 51, 92 and 95 (X) + any one of positions 30, 31, 33, 51, |
| | 92 and 95 not selected for X (Y). |
| B | Each and every unique 3-position combination of X & Y & Z, wherein X, Y, and Z |
| | are each selected (without replacement) from a SEQ ID NO: 460 AA position |
| | indicated in columns 1-6. For example, the combinations of X & Y & Z embodied by |
| | SEQ ID NO: 460 AA position 30, 31, 33, 51, 92 and 95 (X) + any one of positions |
| | 30, 31, 33, 51, 92 and 95 not selected for X (Y) + any one of positions 30, 31, 33, 51, |
| | 92 and 95 not selected for X or Y (Z). |
| C | Each and every unique 4-position combination of X & Y & Z & α, wherein X, Y, Z, |
| | and α are each selected (without replacement) from a SEQ ID NO: 460 AA position |
| | indicated in columns 1-6. For example, the combinations of X & Y & Z & α |
| | embodied by SEQ ID NO: 460 AA position 30, 31, 33, 51, 92 and 95 (X) + any one |
| | of positions 30, 31, 33, 51, 92 and 95 not selected for X (Y) + any one of positions |
| | 30, 31, 33, 51, 92 and 95 not selected for X or Y (Z) + any one of positions 30, 31, |
| | 33, 51, 92 and 95 not selected for X, Y, or Z (α). |

In some examples, the first ABD includes an HCVD that is ≥90% identical to SEQ ID NO: 459, where the HCVD includes a histidine at any of the specific combinations of two or more amino acid positions in SEQ ID NO: 459 listed in Table 11; and an LCVD that that is ≥90% identical to SEQ ID NO: 460, where the LCVD includes a histidine at any of the specific 15 combinations of two or more amino acid positions in SEQ TD NO: 460 listed in Table 12.

In some examples, the first ABD includes an LCVD that is ≥90% identical to SEQ ID NO: 460, where the LCVD includes a histidine at any of the specific combinations of two or more amino acid positions in SEQ ID NO: 460 listed in Table 12 and an HCVD that that is ≥90% identical to SEQ ID NO: 459, where the HCVD includes a histidine at any of the specific combinations of two or more amino acid positions in SEQ ID NO: 459 listed in Table 11.

In some examples, the first ABD comprises an LCVD comprising SEQ ID NO: 460, and an HCVD that is ≥90% identical to SEQ ID NO: 459, where the HCVD includes a histidine at any of the specific combinations of two or more amino acid positions in SEQ ID NO: 459 listed in Table 11.

In some examples, the first ABD comprises an LCVD that is ≥90% identical to SEQ ID NO: 460, where the LCVD includes a histidine at position 30 in SEQ ID NO: 460; and an HCVD that is ≥90% identical to SEQ ID NO: 459, where the HCVD includes a histidine at any of the specific combinations of two or more amino acid positions in SEQ ID NO: 459 listed in Table 11.

In some examples, the first ABD comprises an LCVD that is ≥90% identical to SEQ ID NO: 460, where the LCVD includes a histidine at position 31 in SEQ ID NO: 460; and an HCVD that is ≥90% identical to SEQ ID NO: 459, where the HCVD includes a histidine at any of the specific combinations of two or more amino acid positions in SEQ ID NO: 459 listed in Table 11.

In some examples, the first ABD comprises an LCVD that is at least 90% identical to SEQ ID NO: 460, where the LCVD includes a histidine at position 33 in SEQ ID NO: 460; and an HCVD that is at least 90% identical to SEQ ID NO: 459, where the HCVD includes a histidine at any of the specific combinations of two or more amino acid positions in SEQ ID NO: 459 listed in Table 11.

In some examples, the first ABD comprises an LCVD that is at least 90% identical to SEQ ID NO: 460, wherein the LCVD includes a histidine at position 51 in SEQ ID NO: 460; and an HCVD that is at least 90% identical to SEQ ID NO: 459, wherein the HCVD includes a histidine at any of the specific combinations of two or more amino acid positions in SEQ ID NO: 459 listed in Table 11.

In some examples, the first ABD comprises an LCVD that is ≥90% identical to SEQ ID NO: 460, wherein the LCVD includes a histidine at position 92 in SEQ ID NO: 460; and an HCVD that is at least 90% identical to SEQ ID NO: 459, where the HCVD includes a histidine at any of the specific combinations of two or more amino acid positions in SEQ ID NO: 459 listed in Table 11.

In some examples, the first ABD comprises an LCVD that is ≥90% identical to SEQ ID NO: 460, wherein the LCVD includes a histidine at position 95 in SEQ ID NO: 460; and an HCVD that is at least 90% identical to SEQ ID NO: 459, where the HCVD includes a histidine at any of the specific combinations of two or more amino acid positions in SEQ ID NO: 459 listed in Table 11.

In some examples, the first ABD comprises an HCVD that is ≥90% identical to SEQ ID NO: 459, wherein the HCVD includes a histidine at any one of positions 32, 99, 101, 102, 106 and 111 in SEQ ID NO: 459; and an LCVD that is ≥90% identical to SEQ ID NO: 460, where the LCVD includes a histidine at any of the specific combinations of two or more amino acid positions in SEQ ID NO: 460 listed in Table 12.

In some examples, the first ABD comprises an HCVD of SEQ ID NO: 459, or one of 467-511; and/or the first ABD includes an LCVD of SEQ ID NO: 460, or one of 512-541. In some examples of the ABPCs, the first ABD includes an LCVD comprising any one of SEQ ID NO: 460, or one of 512-541 and an HCVD comprising any one of SEQ ID NO: 459, or one of 467-511.

In some examples, the first ABD includes an LCVD comprising SEQ ID NO: 512, and an HCVD comprising one of: SEQ ID NO: 459, or one of 467-511. In some examples, the first ABD includes an LCVD comprising SEQ ID NO: 513, and an HCVD comprising one of: SEQ ID NO: 459, or one of 467-511. In some examples, the first ABD includes an LCVD comprising SEQ ID NO: 514, and an HCVD comprising one of: SEQ ID NO: 459, or one of 467-511. In some examples, the first ABD includes an LCVD comprising SEQ ID NO: 515, and an HCVD comprising one of: SEQ ID NO: 459, or one of 467-511. In some examples, the first ABD includes an LCVD comprising SEQ ID NO: 516, and an HCVD comprising one of: SEQ ID NO: 459, or one of 467-511. In some examples, the first ABD includes an LCVD comprising SEQ ID NO: 517, and an HCVD comprising one of: SEQ ID NO: 459, or one of 467-511. In some examples, the first ABD includes an LCVD comprising SEQ ID NO: 518, and an HCVD comprising one of: SEQ ID NO: 459, or one of 467-511. In some examples, the first ABD includes an LCVD comprising SEQ ID NO: 519, and an HCVD comprising one of: SEQ ID NO: 459, or one of 467-511. In some examples, the first ABD includes an LCVD comprising SEQ ID NO: 520, and an HCVD comprising one of: SEQ ID NO: 459, or one of 467-511. In some examples, the first ABD includes an LCVD comprising SEQ ID NO: 521, and an HCVD comprising one of: SEQ ID NO: 459, or one of 467-511. In some examples, the first ABD includes an LCVD comprising SEQ ID NO: 522, and an HCVD comprising one of: SEQ ID NO: 459, or one of 467-511. In some examples, the first ABD includes an LCVD comprising SEQ ID NO: 523, and an HCVD comprising one of: SEQ ID NO: 459, or one of 467-511. In some examples, the first ABD includes an LCVD comprising SEQ ID NO: 524, and an HCVD comprising one of: SEQ ID NO: 459, or one of 467-511. In some examples, the first ABD includes an LCVD comprising SEQ ID NO: 525, and an HCVD comprising one of: SEQ ID NO: 459, or one of 467-511. In some examples, the first ABD includes an LCVD comprising SEQ ID NO: 526, and an HCVD comprising one of: SEQ ID NO: 459, or one of 467-511. In some examples, the first ABD includes an LCVD comprising SEQ ID NO: 527, and an HCVD comprising one of: SEQ ID NO: 459, or one of 467-511. In some examples, the first ABD includes an LCVD comprising SEQ ID NO: 528, and an HCVD comprising one of: SEQ ID NO: 459, or one of 467-511. In some examples, the first ABD includes an LCVD comprising SEQ ID NO: 529, and an HCVD comprising one of: SEQ ID NO: 459, or one of 467-511. In some examples, the first ABD includes an LCVD comprising SEQ ID NO: 530, and an HCVD comprising one of: SEQ ID NO: 459, or one of 467-511. In some examples, the first ABD includes an LCVD comprising SEQ ID NO: 531, and an HCVD comprising one of: SEQ ID NO: 459, or one of 467-511. In some examples, the first ABD includes an LCVD comprising SEQ ID NO: 532, and an HCVD comprising one of: SEQ ID NO: 459, or one of 467-511. In some examples, the first ABD includes an LCVD comprising SEQ ID NO: 533, and an HCVD comprising one of: SEQ ID NO: 459, or one of 467-511. In some examples, the first ABD includes an LCVD comprising SEQ ID NO: 534, and an HCVD comprising one of: SEQ ID NO: 459, or one of 467-511. In some examples, the first ABD includes an LCVD comprising SEQ ID NO: 535, and an HCVD comprising one of: SEQ ID NO: 459, or one of 467-511. In some examples, the first ABD includes an LCVD comprising SEQ ID NO: 536, and an HCVD comprising one of: SEQ ID NO: 459, or one of 467-511. In some examples, the first ABD includes an LCVD comprising SEQ TD NO: 537, and an HCVD comprising one of: SEQ ID NO: 459, or one of 467-511. In some examples, the first ABD includes an LCVD comprising SEQ ID NO: 538, and an HCVD comprising one of: SEQ ID NO: 459, or one of 467-511. In some examples, the first ABD includes an LCVD comprising SEQ ID NO: 539, and an HCVD comprising one of: SEQ ID NO: 459, or one of 467-511. In some examples, the first ABD includes an LCVD comprising SEQ ID NO: 540, and an HCVD comprising one of: SEQ ID NO: 459, or one of 467-511. In some examples, the first ABD includes an LCVD comprising SEQ ID NO: 541, and an HCVD comprising one of: SEQ ID NO: 459, or one of 467-511.

In some examples, the first ABD includes an HCVD of MYT9412 with one or more amino acids substituted with a histidine. In some examples, the first ABD includes an LCVD of MYT9412 with one or more amino acids substituted with a histidine. In some examples, the first ABD includes an HCVD of MYT9412 with one or more amino acids optionally substituted with a histidine; and an LCVD of MYT9412 with one or more amino acids substituted with a histidine. In some examples, the HCVD of MYT9412 comprises SEQ ID NO: 542. In some examples, the LCVD of MYT9412 comprises SEQ ID NO: 543.

In some examples, the first ABD comprises an HCVD comprising a CDR1, a CDR2, and a CDR3 of SEQ ID NOs: 544, 545, and 546, respectively, optionally with collectively a total of one or more amino acid positions in SEQ ID NOs: 544, 545, and 546 substituted with a histidine. In some examples, the first ABD comprises an LCVD comprising a CDR1, a CDR2, and a CDR3 of SEQ ID NOs: 547, 548, and 549, respectively, optionally with collectively a total of one or more amino acid positions in SEQ ID NOs: 547, 548, and 549 substituted with a histidine. In some examples, the first ABD includes: an HCVD comprising a CDR1, a CDR2, and a CDR3 of SEQ ID NOs: 544, 545, and 546, respectively, optionally with collectively a total of one or more amino acid positions in NOs: 544, 545, and 546 optionally substituted with a histidine; and an LCVD comprising a CDR1, a CDR2, and a CDR3 of SEQ ID NOs: 547, 548, and 549, respectively, optionally with collectively a total of one or more amino acid positions in SEQ ID NOs: 547, 548, and 549 substituted with a histidine.

In some examples, the first ABD includes an HCVD that is at least 90% identical to SEQ ID NO: 542, where the HCVD optionally includes a histidine at one or more amino acid positions in SEQ ID NO: 542 selected from: 27, 29, 30, 31, 32, 53, 55, 58, 102, 104 and 105. In some examples, the first ABD includes an LCVD that is at least 90% identical to SEQ ID NO: 543, where the LCVD optionally includes a histidine at one or more amino acid positions in SEQ ID NO: 543 selected from: 25, 29, 30, 31, 32, 89, 92 and 98. In some examples, the first ABD includes an HCVD that is at least 90% identical to SEQ ID NO: 542, where the HCVD optionally includes a histidine at one or more amino acid positions in SEQ ID NO: 542 selected from: 27, 29, 30, 31, 32, 53, 55, 58, 102, 104 and 105, and an LCVD that is at least 90% identical to SEQ ID NO: 543, where the LCVD optionally includes a histidine at one or more amino acid positions in SEQ ID NO: 543 selected from: 25, 29, 30, 31, 32, 89, 92 and 98.

In some examples, an HCVD includes an HCVD that is at least 90% identical to SEQ ID NO: 542, where the HCVD includes a histidine at any of the specific combinations of two or more amino acid positions in SEQ ID NO: 542 listed in Table 13.

In some examples, the first ABD includes an HCVD that is ≥90% (e.g., ≥92%, ≥94%, ≥96%, ≥98%, ≥99%, or 100%) identical to SEQ ID NO: 542, where the HCVD includes an alanine at position 55 or 98 in SEQ ID NO: 542.

In some examples the first ABD includes: an HCVD that is ≥90% (e.g., ≥92%, ≥94%, ≥96%, ≥98%, ≥99%, or 100%) identical to SEQ ID NO: 542, where the HCVD includes an alanine at position 55 or 98 in SEQ ID NO: 542, and an LCVD that is ≥90% (e.g., ≥92%, ≥94%, ≥96%, ≥98%, ≥99%, or 100%) identical to SEQ ID NO: 543, where the LCVD optionally includes a histidine at one or more amino acid positions in SEQ ID NO: 543 selected from: 25, 29, 30, 31, 32, 89, 92 and 98.

TABLE 13
Exemplary Combinations of Amino Acid Positions in SEQ ID NO: 542
that can be Substituted with Histidine (or Alanine if indicated)

| Col | 1 | 2 | 3 | 4 | 5 | 6 | 7 | 8 | 9 | 10 | 11 |
|---|---|---|---|---|---|---|---|---|---|---|---|
| Pos | 27 | 29 | 30 | 31 | 32 | 53 | 55 | 58 | 102 | 104 | 105 |

Exemplary 2, 3, and 4 AA position combinations that can be substituted with histidine (or an
alanine at position 55 or 98 of SEQ ID NO: 542)

| | |
|---|---|
| A | Each and every unique 2-position combination of X & Y, wherein X and Y are each |
| | selected (without replacement) from a SEQ ID NO: 542 AA position indicated in |
| | columns 1-11. For example, the combinations of X & Y embodied by SEQ ID NO: |
| | 542 AA position 27, 29, 30, 31, 32, 53, 55, 58, 102, 104 and 105 (X) + any one of |
| | positions 27, 29, 30, 31, 32, 53, 55, 58, 102, 104 and 105 not selected for X (Y). |
| B | Each and every unique 3-position combination of X & Y & Z, wherein X, Y, and Z |
| | are each selected (without replacement) from a SEQ ID NO: 542 AA position |
| | indicated in columns 1-11. For example, the combinations of X & Y & Z embodied by |
| | SEQ ID NO: 542 AA position 27, 29, 30, 31, 32, 53, 55, 58, 102, 104 and 105 (X) + |
| | any one of positions 27, 29, 30, 31, 32, 53, 55, 58, 102, 104 and 105 not selected for X |
| | (Y) + any one of positions 27, 29, 30, 31, 32, 53, 55, 58, 102, 104 and 105 not selected |
| | for X or Y (Z). |
| C | Each and every unique 4-position combination of X & Y & Z & α, wherein X, Y, Z, |
| | and α are each selected (without replacement) from a SEQ ID NO: 542 AA position |
| | indicated in columns 1-11. For example, the combinations of X & Y & Z & α |
| | embodied by SEQ ID NO: 542 AA position 27, 29, 30, 31, 32, 53, 55, 58, 102, 104 |
| | and 105 (X) + any one of positions 27, 29, 30, 31, 32, 53, 55, 58, 102, 104 and 105 not |
| | selected for X (Y) + any one of positions 27, 29, 30, 31, 32, 53, 55, 58, 102, 104 and |
| | 105 not selected for X or Y (Z) + any one of positions 27, 29, 30, 31, 32, 53, 55, 58, |
| | 102, 104 and 105 not selected for X, Y, or Z (α). |

In some examples, the first ABD includes an LCVD that is 90% identical to SEQ ID NO: 543, where the LCVD includes a histidine at any of the specific combinations of two or more amino acid positions in SEQ ID NO: 543 listed in Table 14 and/or where the LCVD includes an alanine at position 98 or 99 of SEQ ID NO: 543.

In some examples, the first ABD includes an LCVD that is ≥90% identical to SEQ ID NO: 543, where the LCVD includes an alanine at position 98 or 99 in SEQ ID NO: 543.

In some examples the first ABD includes: an LCVD that is ≥90% identical to SEQ ID NO: 543, where the LCVD includes an alanine at position 98 or 99 in SEQ ID NO: 543, and an HCVD that is ≥90% identical to SEQ ID NO: 542, where the HCVD optionally includes a histidine at one or more amino acid positions in SEQ ID NO: 542 selected from: 27, 29, 30, 31, 32, 53, 55, 58, 102, 104 and 105.

In some examples, the first ABD includes an LCVD that is ≥90% identical to SEQ ID NO: 543, where the LCVD optionally includes a histidine at one or more amino acid positions in SEQ ID NO: 543 selected from: 25, 29, 30, 31, 32, 89, 92 and 98 and/or where the LCVD includes an alanine at position 98 or 99 in SEQ ID NO: 543. In some examples the first ABD includes: an LCVD that is ≥90% identical to SEQ ID NO: 543, where the LCVD optionally includes a histidine at one or more amino acid positions in SEQ ID NO: 543 selected from: 25, 29, 30, 31, 32, 89, 92 and 98 and/or where the LCVD includes an alanine at position 98 or 99 in SEQ ID NO: 543, and an HCVD that is ≥90% identical to SEQ ID NO: 542, where the HCVD optionally includes a histidine at one or more amino acid positions in SEQ ID NO: 542 selected from: 27, 29, 30, 31, 32, 53, 55, 58, 102, 104 and 105.

In some examples the first ABD includes an LCVD that is ≥90% identical to SEQ TD NO: 543, where the LCVD optionally includes a histidine at one or more amino acid positions in SEQ ID NO: 543 selected from: 25, 29, 30, 31, 32, 89, 92 and 98 and/or where the LCVD includes an alanine at position 98 or 99 in SEQ ID NO: 543, and an HCVD that is 90 identical to SEQ TD NO: 542, where the HCVD optionally includes a histidine at one or more amino acid positions in SEQ ID NO: 542 selected from: 27, 29, 30, 31, 32, 53, 55, 58, 102, 104 and 105 and/or where the HCVD includes an alanine at position 55 or 98.

TABLE 14
Exemplary Combinations of Amino Acid Positions in SEQ ID NO: 543
that can be Substituted with Histidine (or Alanine if indicated)

| Col | 1 | 2 | 3 | 4 | 5 | 6 | 7 | 8 |
|---|---|---|---|---|---|---|---|---|
| Pos | 25 | 29 | 30 | 31 | 32 | 89 | 92 | 98 |

Exemplary 2, 3, and 4 AA position combinations that can be substituted with histidine (or an
alanine at position 98 or 99 of SEQ ID NO: 543)

| | |
|---|---|
| A | Each and every unique 2-position combination of X & Y, wherein X and Y are each |
| | selected (without replacement) from a SEQ ID NO: 543 AA position indicated in |
| | columns 1-8. For example, the combinations of X & Y embodied by SEQ ID NO: |
| | 543 AA position 25, 29, 30, 31, 32, 89, 92 and 98 (X) + any one of positions 25, 29, |
| | 30, 31, 32, 89, 92 and 98 not selected for X (Y). |
| B | Each and every unique 3-position combination of X & Y & Z, wherein X, Y, and Z |
| | are each selected (without replacement) from a SEQ ID NO: 543 AA position |
| | indicated in columns 1-8. For example, the combinations of X & Y & Z embodied by |
| | SEQ ID NO: 543 AA position 25, 29, 30, 31, 32, 89, 92 and 98 (X) + any one of |
| | positions 25, 29, 30, 31, 32, 89, 92 and 98 not selected for X (Y) + any one of |
| | positions 25, 29, 30, 31, 32, 89, 92 and 98 not selected for X or Y (Z). |
| C | Each and every unique 4-position combination of X & Y & Z & α, wherein X, Y, Z, |
| | and α are each selected (without replacement) from a SEQ ID NO: 543 AA position |
| | indicated in columns 1-8. For example, the combinations of X & Y & Z & α |
| | embodied by SEQ ID NO: 543 AA position 25, 29, 30, 31, 32, 89, 92 and 98 (X) + |
| | any one of positions 25, 29, 30, 31, 32, 89, 92 and 98 not selected for X (Y) + any |
| | one of positions 25, 29, 30, 31, 32, 89, 92 and 98 not selected for X or Y (Z) + any |
| | one of positions 25, 29, 30, 31, 32, 89, 92 and 98 not selected for X, Y, or Z (α). |

In some examples, the first ABD includes an HCVD that is ≥90% identical to SEQ ID NO: 542, where the HCVD includes a histidine at any of the specific combinations of two or more amino acid positions in SEQ ID NO: 542 listed in Table 13; and an LCVD that that is ≥90% identical to SEQ ID NO: 543, where the LCVD includes a histidine at any of the specific combinations of two or more amino acid positions in SEQ ID NO: 543 listed in Table 14.

In some examples, the first ABD includes an LCVD that is 90% identical to SEQ ID NO: 543, where the LCVD includes a histidine at any of the specific combinations of two or more amino acid positions in SEQ ID NO: 543 listed in Table 14, and/or where the LCVD includes an alanine at position 98 or 99 in SEQ ID NO: 543; and an HCVD that that is 90% identical to SEQ ID NO: 542, where the HCVD includes a histidine at any of the specific combinations of two or more amino acid positions in SEQ ID NO: 542 listed in Table 13.

In some examples, the first ABD comprises an LCVD comprising SEQ ID NO: 543, and an HCVD that is ≥90% identical to SEQ ID NO: 542, where the HCVD includes a histidine at any of the specific combinations of two or more amino acid positions in SEQ ID NO: 542 listed in Table 13.

In some examples, the first ABD comprises an LCVD that is ≥90% identical to SEQ ID NO: 543, where the LCVD includes a histidine at position 25 in SEQ ID NO: 543; and an HCVD that is ≥90% identical to SEQ ID NO: 542, where the HCVD includes a histidine at any of the specific combinations of two or more amino acid positions in SEQ ID NO: 542 listed in Table 13.

In some examples, the first ABD comprises an LCVD that is ≥90% identical to SEQ ID NO: 543, where the LCVD includes a histidine at position 29 in SEQ ID NO: 543; and an HCVD that is ≥90% identical to SEQ ID NO: 542, where the HCVD includes a histidine at any of the specific combinations of two or more amino acid positions in SEQ ID NO: 542 listed in Table 13.

In some examples, the first ABD comprises an LCVD that is at least 90% identical to SEQ ID NO: 543, where the LCVD includes a histidine at position 30 in SEQ ID NO: 543; and an HCVD that is at least 90% identical to SEQ ID NO: 542, where the HCVD includes a histidine at any of the specific combinations of two or more amino acid positions in SEQ ID NO: 542 listed in Table 13.

In some examples, the first ABD comprises an LCVD that is at least 90% identical to SEQ ID NO: 543, where the LCVD includes an alanine at position 98 or 99 in SEQ ID NO: 543; and an HCVD that is at least 90% identical to SEQ ID NO: 542, wherein the HCVD includes a histidine at any of the specific combinations of two or more amino acid positions in SEQ ID NO: 542 listed in Table 13.

In some examples, the first ABD comprises an LCVD that is at least 90% identical to SEQ ID NO: 543, wherein the LCVD includes a histidine at position 31 in SEQ ID NO: 543; and an HCVD that is at least 90% identical to SEQ ID NO: 542, wherein the HCVD includes a histidine at any of the specific combinations of two or more amino acid positions in SEQ ID NO: 542 listed in Table 13.

In some examples, the first ABD comprises an LCVD that is ≥90% identical to SEQ ID NO: 543, wherein the LCVD includes a histidine at position 32 in SEQ ID NO: 543; and an HCVD that is at least 90% identical to SEQ ID NO: 542, where the HCVD includes a histidine at any of the specific combinations of two or more amino acid positions in SEQ ID NO: 542 listed in Table 13.

In some examples, the first ABD comprises an HCVD that is ≥90% identical to SEQ ID NO: 542, wherein the HCVD includes a histidine at any one of positions 53 in SEQ ID NO: 542; and an LCVD that is ≥90% identical to SEQ ID NO: 543, where the LCVD includes a histidine at any of the specific combinations of two or more amino acid positions in SEQ ID NO: 543 listed in Table 14.

In some examples, the first ABD comprises an HCVD that is ≥90% identical to SEQ ID NO: 542, wherein the HCVD includes a histidine at any one of positions 55 in SEQ ID NO: 542; and an LCVD that is ≥90% identical to SEQ ID NO: 543, where the LCVD includes a histidine at any of the specific combinations of two or more amino acid positions in SEQ ID NO: 543 listed in Table 14.

In some examples, the first ABD comprises an HCVD that is ≥90% identical to SEQ ID NO: 542, wherein the HCVD includes a histidine at any one of positions 58 in SEQ ID NO: 542; and an LCVD that is ≥90% identical to SEQ ID NO: 543, where the LCVD includes a histidine at any of the specific combinations of two or more amino acid positions in SEQ ID NO: 543 listed in Table 14.

In some examples, the first ABD comprises an HCVD that is ≥90% identical to SEQ ID NO: 542, wherein the HCVD includes a histidine at any one of positions 102 in SEQ ID NO: 542; and an LCVD that is ≥90% identical to SEQ ID NO: 543, where the LCVD includes a histidine at any of the specific combinations of two or more amino acid positions in SEQ ID NO: 543 listed in Table 14.

In some examples, the first ABD comprises an HCVD that is ≥90% identical to SEQ ID NO: 542, wherein the HCVD includes a histidine at any one of positions 104 in SEQ ID NO: 542; and an LCVD that is ≥90% identical to SEQ ID NO: 543, where the LCVD includes a histidine at any of the specific combinations of two or more amino acid positions in SEQ ID NO: 543 listed in Table 14.

In some examples, the first ABD comprises an HCVD that is ≥90% identical to SEQ ID NO: 542, wherein the HCVD includes a histidine at any one of positions 105 in SEQ ID NO: 542; and an LCVD that is ≥90% identical to SEQ ID NO: 543, where the LCVD includes a histidine at any of the specific combinations of two or more amino acid positions in SEQ ID NO: 543 listed in Table 14.

In some examples, the first ABD comprises an HCVD of SEQ ID NO: 542, or one of 550-592; and/or the first ABD includes an LCVD of SEQ ID NO: 543, or one of 593-627. In some examples of the ABPCs, the first ABD includes an LCVD comprising any one of SEQ ID NO: 543, or one of 593-627 and an HCVD comprising any one of SEQ ID NO: 542, or one of 550-592.

In some examples, the first ABD includes an LCVD comprising SEQ ID NO: 593, and an HCVD comprising one of: SEQ ID NO: 542, or one of 550-592. In some examples, the first ABD includes an LCVD comprising SEQ ID NO: 594, and an HCVD comprising one of: SEQ ID NO: 542, or one of 550-592. In some examples, the first ABD includes an LCVD comprising SEQ ID NO: 595, and an HCVD comprising one of: SEQ ID NO: 542, or one of 550-592. In some examples, the first ABD includes an LCVD comprising SEQ ID NO: 596, and an HCVD comprising one of: SEQ ID NO: 542, or one of 550-592. In some examples, the first ABD includes an LCVD comprising SEQ ID NO: 597, and an HCVD comprising one of: SEQ ID NO: 542, or one of 550-592. In some examples, the first ABD includes an LCVD comprising SEQ ID NO: 598, and an HCVD comprising one of: SEQ ID NO: 542, or one of 550-592. In some examples, the first ABD includes an LCVD comprising SEQ ID NO: 599, and an HCVD comprising one of: SEQ ID NO: 542, or one of 550-592. In some examples, the first ABD includes an LCVD comprising SEQ ID NO: 600, and an HCVD comprising one of: SEQ ID NO: 542, or one of 550-592. In some examples, the first ABD includes an LCVD comprising SEQ ID NO: 601, and an HCVD comprising one of: SEQ ID NO: 542, or one of 550-592. In some examples, the first ABD includes an LCVD comprising SEQ ID NO: 602, and an HCVD comprising one of: SEQ ID NO: 542, or one of 550-592. In some examples, the first ABD includes an LCVD comprising SEQ ID NO: 603, and an HCVD comprising one of: SEQ ID NO: 542, or one of 550-592. In some examples, the first ABD includes an LCVD comprising SEQ ID NO: 604, and an HCVD comprising one of: SEQ ID NO: 542, or one of 550-592. In some examples, the first ABD includes an LCVD comprising SEQ ID NO: 605, and an HCVD comprising one of: SEQ ID NO: 542, or one of 550-592. In some examples, the first ABD includes an LCVD comprising SEQ ID NO: 606, and an HCVD comprising one of: SEQ ID NO: 542, or one of 550-592. In some examples, the first ABD includes an LCVD comprising SEQ ID NO: 607, and an HCVD comprising one of: SEQ ID NO: 542, or one of 550-592. In some examples, the first ABD includes an LCVD comprising SEQ ID NO: 608, and an HCVD comprising one of: SEQ ID NO: 542, or one of 550-592. In some examples, the first ABD includes an LCVD comprising SEQ ID NO: 609, and an HCVD comprising one of: SEQ ID NO: 542, or one of 550-592. In some examples, the first ABD includes an LCVD comprising SEQ ID NO: 610, and an HCVD comprising one of: SEQ ID NO: 542, or one of 550-592. In some examples, the first ABD includes an LCVD comprising SEQ ID NO: 611, and an HCVD comprising one of: SEQ ID NO: 542, or one of 550-592. In some examples, the first ABD includes an LCVD comprising SEQ ID NO: 612, and an HCVD comprising one of: SEQ ID NO: 542, or one of 550-592. In some examples, the first ABD includes an LCVD comprising SEQ ID NO: 613, and an HCVD comprising one of: SEQ ID NO: 542, or one of 550-592. In some examples, the first ABD includes an LCVD comprising SEQ ID NO: 614, and an HCVD comprising one of: SEQ ID NO: 542, or one of 550-592. In some examples, the first ABD includes an LCVD comprising SEQ ID NO: 615, and an HCVD comprising one of: SEQ ID NO: 542, or one of 550-592. In some examples, the first ABD includes an LCVD comprising SEQ ID NO: 616, and an HCVD comprising one of: SEQ ID NO: 542, or one of 550-592. In some examples, the first ABD includes an LCVD comprising SEQ ID NO: 617, and an HCVD comprising one of: SEQ ID NO: 542, or one of 550-592. In some examples, the first ABD includes an LCVD comprising SEQ ID NO: 618, and an HCVD comprising one of: SEQ ID NO: 542, or one of 550-592. In some examples, the first ABD includes an LCVD comprising SEQ ID NO: 619, and an HCVD comprising one of: SEQ ID NO: 542, or one of 550-592. In some examples, the first ABD includes an LCVD comprising SEQ ID NO: 620, and an HCVD comprising one of: SEQ ID NO: 542, or one of 550-592. In some examples, the first ABD includes an LCVD comprising SEQ TD NO: 621, and an HCVD comprising one of: SEQ ID NO: 542, or one of 550-592. In some examples, the first ABD includes an LCVD comprising SEQ ID NO: 622, and an HCVD comprising one of: SEQ ID NO: 542, or one of 550-592. In some examples, the first ABD includes an LCVD comprising SEQ ID NO: 623, and an HCVD comprising one of: SEQ ID NO: 542, or one of 550-592. In some examples, the first ABD includes an LCVD comprising SEQ ID NO: 624, and an HCVD comprising one of: SEQ ID NO: 542, or one of 550-592. In some examples, the first ABD includes an LCVD comprising SEQ ID NO: 625, and an HCVD comprising one of: SEQ ID NO: 542, or one of 550-592. In some examples, the first ABD includes an LCVD comprising SEQ ID NO: 626, and an HCVD comprising one of: SEQ ID NO: 542, or one of 550-592. In some examples, the first ABD includes an LCVD comprising SEQ ID NO: 627, and an HCVD comprising one of: SEQ ID NO: 542, or one of 550-592.

In some examples, the first ABD includes an HCVD of MYT9460 with one or more amino acids substituted with a histidine. In some examples, the first ABD includes an LCVD of MYT9460 with one or more amino acids substituted with a histidine. In some examples, the first ABD includes an HCVD of MYT9460 with one or more amino acids optionally substituted with a histidine; and an LCVD of MYT9460 with one or more amino acids substituted with a histidine. In some examples, the HCVD of MYT9460 comprises SEQ ID NO: 628. In some examples, the LCVD of MYT9460 comprises SEQ ID NO: 629.

In some examples, the first ABD comprises an HCVD comprising a CDR1, a CDR2, and a CDR3 of SEQ ID NOs: 630, 631, and 632, respectively, optionally with collectively a total of one or more amino acid positions in SEQ ID NOs: 630, 631, and 632 substituted with a histidine. In some examples, the first ABD comprises an LCVD comprising a CDR1, a CDR2, and a CDR3 of SEQ ID NOs: 633, 634, and 635, respectively, optionally with collectively a total of one or more amino acid positions in SEQ ID NOs: 633, 634, and 635 substituted with a histidine. In some examples, the first ABD includes: an HCVD comprising a CDR1, a CDR2, and a CDR3 of SEQ ID NOs: 630, 631, and 632, respectively, optionally with collectively a total of one or more amino acid positions in NOs: 630, 631, and 632 optionally substituted with a histidine; and an LCVD comprising a CDR1, a CDR2, and a CDR3 of SEQ ID NOs: 633, 634, and 635, respectively, optionally with collectively a total of one or more amino acid positions in SEQ ID NOs: 633, 634, and 635 substituted with a histidine.

In some examples, the first ABD includes an HCVD that is at least 90% identical to SEQ ID NO: 628, where the HCVD optionally includes a histidine at one or more amino acid positions in SEQ ID NO: 628 selected from: 26, 28, 30, 50, 52, 53, 54, 55, 56, 57, 100, 101, 102 and 103. In some examples, the first ABD includes an LCVD that is at least 9000 identical to SEQ ID NO: 629, where the LCVD optionally includes a histidine at one or more amino acid positions in SEQ ID NO: 629 selected from: 25, 26, 28, 29, 33, 51, 55, 89, 91, 95, 96, 97, 98, 99 and 100. In some examples, the first ABD includes an HCVD that is at least 90% identical to SEQ ID NO: 628, where the HCVD optionally includes a histidine at one or more amino acid positions in SEQ ID NO: 628 selected from: 26, 28, 30, 50, 52, 53, 54, 55, 56, 57, 100, 101, 102 and 103, and an LCVD that is at least 90 identical to SEQ ID NO: 629, where the LCVD optionally includes a histidine at one or more amino acid positions in SEQ ID NO: 629 selected from: 25, 26, 28, 29, 33, 51, 55, 89, 91, 95, 96, 97, 98, 99 and 100.

In some examples, an HCVD includes an HCVD that is at least 90 identical to SEQ ID NO: 628, where the HCVD includes a histidine at any of the specific combinations of two or more amino acid positions in SEQ ID NO: 628 listed in Table 15.

TABLE 15
Exemplary Combinations of Amino Acid Positions in SEQ
ID NO: 628 that can be Substituted with Histidine

| Col | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| Pos | 26 | 28 | 30 | 50 | 52 | 53 | 54 | 55 | 56 | 57 | 100 | 101 | 102 | 103 |

Exemplary 2, 3, and 4 AA position combinations that can be substituted with histidine

| | |
|---|---|
| A | Each and every unique 2-position combination of X & Y, wherein X and Y are each |
| | selected (without replacement) from a SEQ ID NO: 628 AA position indicated in |
| | columns 1-14. For example, the combinations of X & Y embodied by SEQ ID NO: 628 |
| | AA position 26, 28, 30, 50, 52, 53, 54, 55, 56, 57, 100, 101, 102 and 103 (X) + any one |
| | of positions 26, 28, 30, 50, 52, 53, 54, 55, 56, 57, 100, 101, 102 and 103 not selected |
| | for X (Y). |
| B | Each and every unique 3-position combination of X & Y & Z, wherein X, Y, and Z are |
| | each selected (without replacement) from a SEQ ID NO: 628 AA position indicated in |
| | columns 1-14. For example, the combinations of X & Y & Z embodied by SEQ ID |
| | NO: 628 AA position 26, 28, 30, 50, 52, 53, 54, 55, 56, 57, 100, 101, 102 and 103 (X) + |
| | any one of positions 26, 28, 30, 50, 52, 53, 54, 55, 56, 57, 100, 101, 102 and 103 not |
| | selected for X (Y) + any one of positions 26, 28, 30, 50, 52, 53, 54, 55, 56, 57, 100, |
| | 101, 102 and 103 not selected for X or Y (Z). |
| C | Each and every unique 4-position combination of X & Y & Z & α, wherein X, Y, Z, |
| | and α are each selected (without replacement) from a SEQ ID NO: 628 AA position |
| | indicated in columns 1-14. For example, the combinations of X & Y & Z & α |
| | embodied by SEQ ID NO: 628 AA position 26, 28, 30, 50, 52, 53, 54, 55, 56, 57, 100, |
| | 101, 102 and 103 (X) + any one of positions 26, 28, 30, 50, 52, 53, 54, 55, 56, 57, 100, |
| | 101, 102 and 103 not selected for X (Y) + any one of positions 26, 28, 30, 50, 52, 53, |
| | 54, 55, 56, 57, 100, 101, 102 and 103 not selected for X or Y (Z) + any one of positions |
| | 26, 28, 30, 50, 52, 53, 54, 55, 56, 57, 100, 101, 102 and 103 not selected for X, Y, or Z |
| | (α). |

In some examples, the first ABD includes an LCVD that is ≥90% identical to SEQ ID NO: 629, where the LCVD includes a histidine at any of the specific combinations of two or more amino acid positions in SEQ ID NO: 629 listed in Table 16 and/or where the LCVD includes an alanine at position 96 of SEQ ID NO: 629.

In some examples, the first ABD includes an LCVD that is 90% identical to SEQ ID NO: 629, where the LCVD includes an alanine at position 96 in SEQ ID NO: 629.

In some examples the first ABD includes: an LCVD that is 90% identical to SEQ ID NO: 629, where the LCVD includes an alanine at position 96 in SEQ ID NO: 629, and an HCVD that is ≥90% identical to SEQ ID NO: 628, where the HCVD optionally includes a histidine at one or more amino acid positions in SEQ ID NO: 628 selected from: 26, 28, 30, 50, 52, 53, 54, 55, 56, 57, 100, 101, 102 and 103.

In some examples, the first ABD includes an LCVD that is 90% identical to SEQ ID NO: 629, where the LCVD optionally includes a histidine at one or more amino acid positions in SEQ ID NO: 629 selected from: 25, 26, 28, 29, 33, 51, 55, 89, 91, 95, 96, 97, 98, 99 and 100 and/or where the LCVD includes an alanine at position 96 in SEQ ID NO: 629. In some examples the first ABD includes: an LCVD that is ≥90% identical to SEQ ID NO: 629, where the LCVD optionally includes a histidine at one or more amino acid positions in SEQ ID NO: 629 selected from: 25, 26, 28, 29, 33, 51, 55, 89, 91, 95, 96, 97, 98, 99 and 100 and/or where the LCVD includes an alanine at position 96 in SEQ ID NO: 629, and an HCVD that is ≥90% identical to SEQ ID NO: 628, where the HCVD optionally includes a histidine at one or more amino acid positions in SEQ ID NO: 628 selected from: 26, 28, 30, 50, 52, 53, 54, 55, 56, 57, 100, 101, 102 and 103.

In some examples the first ABD includes an LCVD that is ≥90% identical to SEQ ID NO: 629, where the LCVD optionally includes a histidine at one or more amino acid positions in SEQ ID NO: 629 selected from: 25, 26, 28, 29, 33, 51, 55, 89, 91, 95, 96, 97, 98, 99 and 100 and/or where the LCVD includes an alanine at position 96 in SEQ ID NO: 629, and an HCVD that is ≥902 identical to SEQ ID NO: 628, where the HCVD optionally includes a histidine at one or more amino acid positions in SEQ TD NO: 628 selected from: 26, 28, 30, 50, 52, 53, 54, 55, 56, 57, 100, 101, 102 and 103.

TABLE 16
Exemplary Combinations of Amino Acid Positions in SEQ ID NO: 629
that can be Substituted with Histidine (or Alanine if indicated)

| Col | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| Pos | 25 | 26 | 28 | 29 | 33 | 51 | 55 | 89 | 91 | 95 | 96 | 97 | 98 | 99 | 100 |

Exemplary 2, 3, and 4 AA position combinations that can be substituted
with histidine (or an alanine at position 96 of SEQ ID NO: 629)

| | |
|---|---|
| A | Each and every unique 2-position combination of X & Y, wherein X and Y are each |
| | selected (without replacement) from a SEQ ID NO: 629 AA position indicated in |
| | columns 1-15. For example, the combinations of X & Y embodied by SEQ ID NO: |
| | 629 AA position 25, 26, 28, 29, 33, 51, 55, 89, 91, 95, 96, 97, 98, 99 and 100 (X) + |
| | any one of positions 25, 26, 28, 29, 33, 51, 55, 89, 91, 95, 96, 97, 98, 99 and 100 not |
| | selected for X (Y). |
| B | Each and every unique 3-position combination of X & Y & Z, wherein X, Y, and Z are |
| | each selected (without replacement) from a SEQ ID NO: 629 AA position indicated in |
| | columns 1-15. For example, the combinations of X & Y & Z embodied by SEQ ID |
| | NO: 629 AA position 25, 26, 28, 29, 33, 51, 55, 89, 91, 95, 96, 97, 98, 99 and 100 (X) + |
| | any one of positions 25, 26, 28, 29, 33, 51, 55, 89, 91, 95, 96, 97, 98, 99 and 100 not |
| | selected for X (Y) + any one of positions 25, 26, 28, 29, 33, 51, 55, 89, 91, 95, 96, 97, |
| | 98, 99 and 100 not selected for X or Y (Z). |
| C | Each and every unique 4-position combination of X & Y & Z & α, wherein X, Y, Z, |
| | and α are each selected (without replacement) from a SEQ ID NO: 629 AA position |
| | indicated in columns 1-15. For example, the combinations of X & Y & Z & α |
| | embodied by SEQ ID NO: 629 AA position 25, 26, 28, 29, 33, 51, 55, 89, 91, 95, 96, |
| | 97, 98, 99 and 100 (X) + any one of positions 25, 26, 28, 29, 33, 51, 55, 89, 91, 95, 96, |
| | 97, 98, 99 and 100 not selected for X (Y) + any one of positions 25, 26, 28, 29, 33, 51, |
| | 55, 89, 91, 95, 96, 97, 98, 99 and 100 not selected for X or Y (Z) + any one of |
| | positions 25, 26, 28, 29, 33, 51, 55, 89, 91, 95, 96, 97, 98, 99 and 100 not selected for |
| | X, Y, or Z (α). |

In some examples, the first ABD includes an HCVD that is ≥90% identical to SEQ ID NO: 628, where the HCVD includes a histidine at any of the specific combinations of two or more amino acid positions in SEQ ID NO: 628 listed in Table 15; and an LCVD that that is ≥90% identical to SEQ ID NO: 629, where the LCVD includes a histidine at any of the specific combinations of two or more amino acid positions in SEQ TD NO: 629 listed in Table 16.

In some examples, the first ABD includes an LCVD that is 90% identical to SEQ ID NO: 629, where the LCVD includes a histidine at any of the specific combinations of two or more amino acid positions in SEQ ID NO: 629 listed in Table 16, and/or where the LCVD includes an alanine at position 96 in SEQ ID NO: 629; and an HCVD that that is ≥90% identical to SEQ ID NO: 628, where the HCVD includes a histidine at any of the specific combinations of two or more amino acid positions in SEQ ID NO: 628 listed in Table 15.

In some examples, the first ABD comprises an LCVD comprising SEQ ID NO: 629, and an HCVD that is ≥90% identical to SEQ ID NO: 628, where the HCVD includes a histidine at any of the specific combinations of two or more amino acid positions in SEQ ID NO: 628 listed in Table 15.

In some examples, the first ABD comprises an LCVD that is ≥90% identical to SEQ ID NO: 629, where the LCVD includes a histidine at position 25 in SEQ ID NO: 629; and an HCVD that is ≥90% identical to SEQ ID NO: 628, where the HCVD includes a histidine at any of the specific combinations of two or more amino acid positions in SEQ ID NO: 628 listed in Table 15.

In some examples, the first ABD comprises an LCVD that is ≥90% identical to SEQ ID NO: 629, where the LCVD includes a histidine at position 26 in SEQ ID NO: 629; and an HCVD that is ≥90% identical to SEQ ID NO: 628, where the HCVD includes a histidine at any of the specific combinations of two or more amino acid positions in SEQ ID NO: 628 listed in Table 15.

In some examples, the first ABD comprises an LCVD that is ≥90% identical to SEQ ID NO: 629, where the LCVD includes a histidine at position 28 in SEQ ID NO: 629; and an HCVD that is ≥90% identical to SEQ ID NO: 628, where the HCVD includes a histidine at any of the specific combinations of two or more amino acid positions in SEQ ID NO: 628 listed in Table 15.

In some examples, the first ABD comprises an LCVD that is ≥90% identical to SEQ ID NO: 629, where the LCVD includes a histidine at position 29 in SEQ ID NO: 629; and an HCVD that is ≥90% identical to SEQ ID NO: 628, where the HCVD includes a histidine at any of the specific combinations of two or more amino acid positions in SEQ ID NO: 628 listed in Table 15.

In some examples, the first ABD comprises an LCVD that is ≥90% identical to SEQ ID NO: 629, where the LCVD includes a histidine at position 33 in SEQ ID NO: 629; and an HCVD that is ≥90% identical to SEQ ID NO: 628, where the HCVD includes a histidine at any of the specific combinations of two or more amino acid positions in SEQ ID NO: 628 listed in Table 15.

In some examples, the first ABD comprises an LCVD that is ≥90% identical to SEQ ID NO: 629, where the LCVD includes a histidine at position 51 in SEQ ID NO: 629; and an HCVD that is ≥90% identical to SEQ ID NO: 628, where the HCVD includes a histidine at any of the specific combinations of two or more amino acid positions in SEQ ID NO: 628 listed in Table 15.

In some examples, the first ABD comprises an LCVD that is at least 90% identical to SEQ ID NO: 629, where the LCVD includes a histidine at position 55 in SEQ ID NO: 629; and an HCVD that is at least 90% identical to SEQ ID NO: 628, where the HCVD includes a histidine at any of the specific combinations of two or more amino acid positions in SEQ ID NO: 628 listed in Table 15.

In some examples, the first ABD comprises an LCVD that is at least 90% identical to SEQ ID NO: 629, where the LCVD includes an alanine at position 96 in SEQ ID NO: 629; and an HCVD that is at least 90% identical to SEQ ID NO: 628, wherein the HCVD includes a histidine at any of the specific combinations of two or more amino acid positions in SEQ ID NO: 628 listed in Table 15.

In some examples, the first ABD comprises an LCVD that is at least 90% identical to SEQ ID NO: 629, wherein the LCVD includes a histidine at position 89 in SEQ ID NO: 629; and an HCVD that is at least 90% identical to SEQ ID NO: 628, wherein the HCVD includes a histidine at any of the specific combinations of two or more amino acid positions in SEQ ID NO: 628 listed in Table 15.

In some examples, the first ABD comprises an LCVD that is ≥90% identical to SEQ ID NO: 629, wherein the LCVD includes a histidine at position 91 in SEQ ID NO: 629; and an HCVD that is at least 90% identical to SEQ ID NO: 628, where the HCVD includes a histidine at any of the specific combinations of two or more amino acid positions in SEQ ID NO: 628 listed in Table 15.

In some examples, the first ABD comprises an LCVD that is ≥90% identical to SEQ ID NO: 629, wherein the LCVD includes a histidine at position 95 in SEQ ID NO: 629; and an HCVD that is at least 90% identical to SEQ ID NO: 628, where the HCVD includes a histidine at any of the specific combinations of two or more amino acid positions in SEQ ID NO: 628 listed in Table 15.

In some examples, the first ABD comprises an LCVD that is ≥90% identical to SEQ ID NO: 629, wherein the LCVD includes a histidine at position 96 in SEQ ID NO: 629; and an HCVD that is at least 90% identical to SEQ ID NO: 628, where the HCVD includes a histidine at any of the specific combinations of two or more amino acid positions in SEQ ID NO: 628 listed in Table 15.

In some examples, the first ABD comprises an LCVD that is ≥90% identical to SEQ ID NO: 629, wherein the LCVD includes a histidine at position 97 in SEQ ID NO: 629; and an HCVD that is at least 90% identical to SEQ ID NO: 628, where the HCVD includes a histidine at any of the specific combinations of two or more amino acid positions in SEQ ID NO: 628 listed in Table 15.

In some examples, the first ABD comprises an LCVD that is ≥90% identical to SEQ ID NO: 629, wherein the LCVD includes a histidine at position 98 in SEQ ID NO: 629; and an HCVD that is at least 90% identical to SEQ ID NO: 628, where the HCVD includes a histidine at any of the specific combinations of two or more amino acid positions in SEQ ID NO: 628 listed in Table 15.

In some examples, the first ABD comprises an LCVD that is ≥90% identical to SEQ ID NO: 629, wherein the LCVD includes a histidine at position 99 in SEQ ID NO: 629; and an HCVD that is at least 90% identical to SEQ ID NO: 628, where the HCVD includes a histidine at any of the specific combinations of two or more amino acid positions in SEQ ID NO: 628 listed in Table 15.

In some examples, the first ABD comprises an LCVD that is ≥90% identical to SEQ ID NO: 629, wherein the LCVD includes a histidine at position 100 in SEQ ID NO: 629; and an HCVD that is at least 90% identical to SEQ ID NO: 628, where the HCVD includes a histidine at any of the specific combinations of two or more amino acid positions in SEQ ID NO: 628 listed in Table 15.

In some examples, the first ABD comprises an HCVD that is ≥90% identical to SEQ ID NO: 628, wherein the HCVD includes a histidine at any one of positions 26, 28, 30, 50, 52, 53, 54, 55, 56, 57, 100, 101, 102 and 103 in SEQ ID NO: 628; and an LCVD that is ≥90% identical to SEQ ID NO: 629, where the LCVD includes a histidine at any of the specific combinations of two or more amino acid positions in SEQ ID NO: 629 listed in Table 16.

In some examples, the first ABD comprises an HCVD of SEQ ID NO: 628, or one of 636-675; and/or the first ABD includes an LCVD of SEQ ID NO: 629, or one of 676-709. In some examples of the ABPCs, the first ABD includes an LCVD comprising any one of SEQ ID NO: 629, or one of 676-709 and an HCVD comprising any one of SEQ ID NO: 628, or one of 636-675.

In some examples, the first ABD includes an LCVD comprising SEQ ID NO: 676, and an HCVD comprising one of: SEQ ID NO: 628, or one of 636-675. In some examples, the first ABD includes an LCVD comprising SEQ ID NO: 677, and an HCVD comprising one of: SEQ ID NO: 628, or one of 636-675. In some examples, the first ABD includes an LCVD comprising SEQ ID NO: 678, and an HCVD comprising one of: SEQ ID NO: 628, or one of 636-675. In some examples, the first ABD includes an LCVD comprising SEQ ID NO: 679, and an HCVD comprising one of: SEQ ID NO: 628, or one of 636-675. In some examples, the first ABD includes an LCVD comprising SEQ ID NO: 680, and an HCVD comprising one of: SEQ ID NO: 628, or one of 636-675. In some examples, the first ABD includes an LCVD comprising SEQ ID NO: 681, and an HCVD comprising one of: SEQ ID NO: 628, or one of 636-675. In some examples, the first ABD includes an LCVD comprising SEQ ID NO: 682, and an HCVD comprising one of: SEQ ID NO: 628, or one of 636-675. In some examples, the first ABD includes an LCVD comprising SEQ ID NO: 683, and an HCVD comprising one of: SEQ ID NO: 628, or one of 636-675. In some examples, the first ABD includes an LCVD comprising SEQ ID NO: 684, and an HCVD comprising one of: SEQ ID NO: 628, or one of 636-675. In some examples, the first ABD includes an LCVD comprising SEQ ID NO: 685, and an HCVD comprising one of: SEQ ID NO: 628, or one of 636-675. In some examples, the first ABD includes an LCVD comprising SEQ ID NO: 686, and an HCVD comprising one of: SEQ ID NO: 628, or one of 636-675. In some examples, the first ABD includes an LCVD comprising SEQ ID NO: 687, and an HCVD comprising one of: SEQ ID NO: 628, or one of 636-675. In some examples, the first ABD includes an LCVD comprising SEQ ID NO: 688, and an HCVD comprising one of: SEQ ID NO: 628, or one of 636-675. In some examples, the first ABD includes an LCVD comprising SEQ ID NO: 689, and an HCVD comprising one of: SEQ ID NO: 628, or one of 636-675. In some examples, the first ABD includes an LCVD comprising SEQ ID NO: 690, and an HCVD comprising one of: SEQ ID NO: 628, or one of 636-675. In some examples, the first ABD includes an LCVD comprising SEQ ID NO: 691, and an HCVD comprising one of: SEQ ID NO: 628, or one of 636-675. In some examples, the first ABD includes an LCVD comprising SEQ ID NO: 692, and an HCVD comprising one of: SEQ ID NO: 628, or one of 636-675. In some examples, the first ABD includes an LCVD comprising SEQ ID NO: 693, and an HCVD comprising one of: SEQ ID NO: 628, or one of 636-675. In some examples, the first ABD includes an LCVD comprising SEQ ID NO: 694, and an HCVD comprising one of: SEQ ID NO: 628, or one of 636-675. In some examples, the first ABD includes an LCVD comprising SEQ ID NO: 695, and an HCVD comprising one of: SEQ ID NO: 628, or one of 636-675. In some examples, the first ABD includes an LCVD comprising SEQ ID NO: 696, and an HCVD comprising one of: SEQ ID NO: 628, or one of 636-675. In some examples, the first ABD includes an LCVD comprising SEQ ID NO: 697, and an HCVD comprising one of: SEQ ID NO: 628, or one of 636-675. In some examples, the first ABD includes an LCVD comprising SEQ ID NO: 698, and an HCVD comprising one of: SEQ ID NO: 628, or one of 636-675. In some examples, the first ABD includes an LCVD comprising SEQ ID NO: 699, and an HCVD comprising one of: SEQ ID NO: 628, or one of 636-675. In some examples, the first ABD includes an LCVD comprising SEQ ID NO: 700, and an HCVD comprising one of: SEQ ID NO: 628, or one of 636-675. In some examples, the first ABD includes an LCVD comprising SEQ ID NO: 701, and an HCVD comprising one of: SEQ ID NO: 628, or one of 636-675. In some examples, the first ABD includes an LCVD comprising SEQ ID NO: 702, and an HCVD comprising one of: SEQ ID NO: 628, or one of 636-675. In some examples, the first ABD includes an LCVD comprising SEQ ID NO: 703, and an HCVD comprising one of: SEQ ID NO: 628, or one of 636-675. In some examples, the first ABD includes an LCVD comprising SEQ ID NO: 704, and an HCVD comprising one of: SEQ ID NO: 628, or one of 636-675. In some examples, the first ABD includes an LCVD comprising SEQ ID NO: 705, and an HCVD comprising one of: SEQ ID NO: 628, or one of 636-675. In some examples, the first ABD includes an LCVD comprising SEQ ID NO: 706, and an HCVD comprising one of: SEQ ID NO: 628, or one of 636-675. In some examples, the first ABD includes an LCVD comprising SEQ TD NO: 707, and an HCVD comprising one of: SEQ ID NO: 628, or one of 636-675. In some examples, the first ABD includes an LCVD comprising SEQ ID NO: 708, and an HCVD comprising one of: SEQ ID NO: 628, or one of 636-675. In some examples, the first ABD includes an LCVD comprising SEQ ID NO: 709, and an HCVD comprising one of: SEQ ID NO: 628, or one of 636-675.

In some examples, the first ABD includes an HCVD of MYT9792 with one or more amino acids substituted with a histidine. In some examples, the first ABD includes an LCVD of MYT9792 with one or more amino acids substituted with a histidine. In some examples, the first ABD includes an HCVD of MYT9792 with one or more amino acids optionally substituted with a histidine; and an LCVD of MYT9792 with one or more amino acids substituted with a histidine. In some examples, the HCVD of MYT9792 comprises SEQ ID NO: 710. In some examples, the LCVD of MYT9792 comprises SEQ ID NO: 711.

In some examples, the first ABD comprises an HCVD comprising a CDR1, a CDR2, and a CDR3 of SEQ ID NOs: 712, 713, and 714, respectively, optionally with collectively a total of one or more amino acid positions in SEQ ID NOs: 712, 713, and 714 substituted with a histidine. In some examples, the first ABD comprises an LCVD comprising a CDR1, a CDR2, and a CDR3 of SEQ ID NOs: 715, 716, and 717, respectively, optionally with collectively a total of one or more amino acid positions in SEQ ID NOs: 715, 716, and 717 substituted with a histidine. In some examples, the first ABD includes: an HCVD comprising a CDR1, a CDR2, and a CDR3 of SEQ ID NOs: 712, 713, and 714, respectively, optionally with collectively a total of one or more amino acid positions in NOs: 712, 713, and 714 substituted with a histidine; and an LCVD comprising a CDR1, a CDR2, and a CDR3 of SEQ ID NOs: 715, 716, and 717, respectively, optionally with collectively a total of one or more amino acid positions in SEQ ID NOs: 715, 716, and 717 substituted with a histidine.

In some examples, the first ABD includes an HCVD that is at least 90% identical to SEQ ID NO: 710, where the HCVD optionally includes a histidine at one or more amino acid positions in SEQ ID NO: 710 selected from: 29, 30, 31, 33, 35, 50, 53, 57, 58, 65, 96, 97, 98, 100, 101, 103, 105, 107, 108, 109, 110 and 113. In some examples, the first ABD includes an LCVD that is at least 90% identical to SEQ ID NO: 711, where the LCVD optionally includes a histidine at one or more amino acid positions in SEQ ID NO: 711 selected from: 25, 27, 29, 30, 35, 36, 56, 57, 91, 93, 94, 95, 98 and 99. In some examples, the first ABD includes an HCVD that is at least 90% identical to SEQ ID NO: 710, where the HCVD optionally includes a histidine at one or more amino acid positions in SEQ ID NO: 710 selected from: 29, 30, 31, 33, 35, 50, 53, 57, 58, 65, 96, 97, 98, 100, 101, 103, 105, 107, 108, 109, 110 and 113, and an LCVD that is at least 90% identical to SEQ ID NO: 711, where the LCVD optionally includes a histidine at one or more amino acid positions in SEQ ID NO: 711 selected from: 25, 27, 29, 30, 35, 36, 56, 57, 91, 93, 94, 95, 98 and 99.

In some examples, an HCVD includes an HCVD that is at least 90% identical to SEQ ID NO: 710, where the HCVD includes a histidine at any of the specific combinations of two or more amino acid positions in SEQ ID NO: 710 listed in Table 17.

In some examples, the first ABD includes an HCVD that is ≥90% (e.g., ≥92%, ≥94%, ≥96%, ≥98%, ≥99%, or 100%) identical to SEQ ID NO: 710, where the HCVD includes an alanine at position 114 in SEQ ID NO: 710. In some examples the first ABD includes: an HCVD that is ≥90% (e.g., ≥92%, ≥94%, ≥96%, ≥98%, ≥99%, or 100%) identical to SEQ ID NO: 710, where the HCVD includes an alanine at position 114 in SEQ ID NO: 710, and an LCVD that is ≥90% (e.g., ≥92%, ≥94%, ≥96%, ≥98%, ≥99%, or 100%) identical to SEQ ID NO: 711, where the LCVD optionally includes a histidine at one or more amino acid positions in SEQ ID NO: 711 selected from: 25, 27, 29, 30, 35, 36, 56, 57, 91, 93, 94, 95, 98 and 99.

TABLE 17
Exemplary Combinations of Amino Acid Positions in SEQ ID NO: 710
that can be Substituted with Histidine (or Alanine if indicated)

| 1 | 2 | 3 | 4 | 5 | 6 | 7 | 8 | 9 | 10 | 11 |
|---|---|---|---|---|---|---|---|---|---|---|
| 29 | 30 | 31 | 33 | 35 | 50 | 53 | 57 | 58 | 65 | 96 |
| 12 | 13 | 14 | 15 | 16 | 17 | 18 | 19 | 20 | 21 | 22 |
| 97 | 98 | 100 | 101 | 103 | 105 | 107 | 108 | 109 | 110 | 113 |

Exemplary 2, 3, and 4 AA position combinations that can be substituted
with histidine (or an alanine at position 114 of SEQ ID NO: 710)

| | |
|---|---|
| A | Each and every unique 2-position combination of X & Y, wherein X and Y are each |
| | selected (without replacement) from a SEQ ID NO: 710 AA position indicated in |
| | columns 1-22. For example, the combinations of X & Y embodied by SEQ ID NO: |
| | 710 AA position 29, 30, 31, 33, 35, 50, 53, 57, 58, 65, 96, 97, 98, 100, 101, 103, 105, |
| | 107, 108, 109, 110 and 113 (X) + any one of positions 29, 30, 31, 33, 35, 50, 53, 57, |
| | 58, 65, 96, 97, 98, 100, 101, 103, 105, 107, 108, 109, 110 and 113 not selected for X |
| | (Y). |
| B | Each and every unique 3-position combination of X & Y & Z, wherein X, Y, and Z |
| | are each selected (without replacement) from a SEQ ID NO: 710 AA position |
| | indicated in columns 1-22. For example, the combinations of X & Y & Z embodied |
| | by SEQ ID NO: 710 AA position 29, 30, 31, 33, 35, 50, 53, 57, 58, 65, 96, 97, 98, |
| | 100, 101, 103, 105, 107, 108, 109, 110 and 113 (X) + any one of positions 29, 30, 31, |
| | 33, 35, 50, 53, 57, 58, 65, 96, 97, 98, 100, 101, 103, 105, 107, 108, 109, 110 and 113 |
| | not selected for X (Y) + any one of positions 29, 30, 31, 33, 35, 50, 53, 57, 58, 65, |
| | 96, 97, 98, 100, 101, 103, 105, 107, 108, 109, 110 and 113 not selected for X or Y |
| | (Z). |
| C | Each and every unique 4-position combination of X & Y & Z & α, wherein X, Y, Z, |
| | and α are each selected (without replacement) from a SEQ ID NO: 710 AA position |
| | indicated in columns 1-22. For example, the combinations of X & Y & Z & α |
| | embodied by SEQ ID NO: 710 AA position 29, 30, 31, 33, 35, 50, 53, 57, 58, 65, 96, |
| | 97, 98, 100, 101, 103, 105, 107, 108, 109, 110 and 113 (X) + any one of positions 29, |
| | 30, 31, 33, 35, 50, 53, 57, 58, 65, 96, 97, 98, 100, 101, 103, 105, 107, 108, 109, 110 |
| | and 113 not selected for X (Y) + any one of positions 29, 30, 31, 33, 35, 50, 53, 57, |
| | 58, 65, 96, 97, 98, 100, 101, 103, 105, 107, 108, 109, 110 and 113 not selected for X |
| | or Y (Z) + any one of positions 29, 30, 31, 33, 35, 50, 53, 57, 58, 65, 96, 97, 98, 100, |
| | 101, 103, 105, 107, 108, 109, 110 and 113 not selected for X, Y, or Z (α). |

In some examples, the first ABD includes an LCVD that is ≥90% identical to SEQ ID NO: 711, where the LCVD includes a histidine at any of the specific combinations of two or more amino acid positions in SEQ ID NO: 711 listed in Table 18 and/or where the LCVD includes an alanine at position 99 of SEQ ID NO: 711.

In some examples, the first ABD includes an LCVD that is 90% identical to SEQ ID NO: 711, where the LCVD includes an alanine at position 99 in SEQ ID NO: 711.

In some examples the first ABD includes: an LCVD that is ≥90% identical to SEQ ID NO: 711, where the LCVD includes an alanine at position 99 in SEQ ID NO: 711, and an HCVD that is ≥90% identical to SEQ ID NO: 710, where the HCVD optionally includes a histidine at one or more amino acid positions in SEQ ID NO: 710 selected from: 29, 30, 31, 33, 35, 50, 53, 57, 58, 65, 96, 97, 98, 100, 101, 103, 105, 107, 108, 109, 110 and 113.

In some examples, the first ABD includes an LCVD that is ≥90% identical to SEQ ID NO: 711, where the LCVD optionally includes a histidine at one or more amino acid positions in SEQ ID NO: 711 selected from: 25, 27, 29, 30, 35, 36, 56, 57, 91, 93, 94, 95, 98 and 99 and/or where the LCVD includes an alanine at position 99 in SEQ ID NO: 711. In some examples the first ABD includes: an LCVD that is ≥90% identical to SEQ ID NO: 711, where the LCVD optionally includes a histidine at one or more amino acid positions in SEQ ID NO: 711 selected from: 25, 27, 29, 30, 35, 36, 56, 57, 91, 93, 94, 95, 98 and 99 and/or where the LCVD includes an alanine at position 99 in SEQ ID NO: 711, and an HCVD that is ≥90% identical to SEQ TD NO: 710, where the HCVD optionally includes a histidine at one or more amino acid positions in SEQ ID NO: 710 selected from: 29, 30, 31, 33, 35, 50, 53, 57, 58, 65, 96, 97, 98, 100, 101, 103, 105, 107, 108, 109, 110 and 113.

In some examples the first ABD includes an LCVD that is 900 identical to SEQ ID NO: 711, where the LCVD optionally includes a histidine at one or more amino acid positions in SEQ ID NO: 711 selected from: 25, 27, 29, 30, 35, 36, 56, 57, 91, 93, 94, 95, 98 and 99 and/or where the LCVD includes an alanine at position 99 in SEQ ID NO: 711, and an HCVD that is ≥90% identical to SEQ ID NO: 710, where the HCVD optionally includes a histidine at one or more amino acid positions in SEQ TD NO: 710 selected from: 29, 30, 31, 33, 35, 50, 53, 57, 58, 65, 96, 97, 98, 100, 101, 103, 105, 107, 108, 109, 110 and 113 and/or where the HCVD includes an alanine at position 114.

TABLE 18
Exemplary Combinations of Amino Acid Positions in SEQ ID NO: 711
that can be Substituted with Histidine (or Alanine if indicated)

| Col | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| Pos | 25 | 27 | 29 | 30 | 35 | 36 | 56 | 57 | 91 | 93 | 94 | 95 | 98 | 99 |

Exemplary 2, 3, and 4 AA position combinations that can be substituted
with histidine (or an alanine at position 99 of SEQ ID NO: 711)

| | |
|---|---|
| A | Each and every unique 2-position combination of X & Y, wherein X and Y are each |
| | selected (without replacement) from a SEQ ID NO: 711 AA position indicated in |
| | columns 1-14. For example, the combinations of X & Y embodied by SEQ ID NO: |
| | 711 AA position 25, 27, 29, 30, 35, 36, 56, 57, 91, 93, 94, 95, 98 and 99 (X) + any one |
| | of positions 25, 27, 29, 30, 35, 36, 56, 57, 91, 93, 94, 95, 98 and 99 not selected for X |
| | (Y). |
| B | Each and every unique 3-position combination of X & Y & Z, wherein X, Y, and Z are |
| | each selected (without replacement) from a SEQ ID NO: 711 AA position indicated in |
| | columns 1-14. For example, the combinations of X & Y & Z embodied by SEQ ID |
| | NO: 711 AA position 25, 27, 29, 30, 35, 36, 56, 57, 91, 93, 94, 95, 98 and 99 (X) + |
| | any one of positions 25, 27, 29, 30, 35, 36, 56, 57, 91, 93, 94, 95, 98 and 99 not |
| | selected for X (Y) + any one of positions 25, 27, 29, 30, 35, 36, 56, 57, 91, 93, 94, 95, |
| | 98 and 99 not selected for X or Y (Z). |
| C | Each and every unique 4-position combination of X & Y & Z & α, wherein X, Y, Z, |
| | and α are each selected (without replacement) from a SEQ ID NO: 711 AA position |
| | indicated in columns 1-14. For example, the combinations of X & Y & Z & α |
| | embodied by SEQ ID NO: 711 AA position 25, 27, 29, 30, 35, 36, 56, 57, 91, 93, 94, |
| | 95, 98 and 99 (X) + any one of positions 25, 27, 29, 30, 35, 36, 56, 57, 91, 93, 94, 95, |
| | 98 and 99 not selected for X (Y) + any one of positions 25, 27, 29, 30, 35, 36, 56, 57, |
| | 91, 93, 94, 95, 98 and 99 not selected for X or Y (Z) + any one of positions 25, 27, 29, |
| | 30, 35, 36, 56, 57, 91, 93, 94, 95, 98 and 99 not selected for X, Y, or Z (α). |

In some examples, the first ABD includes an HCVD that is ≥90% identical to SEQ ID NO: 710, where the HCVD includes a histidine at any of the specific combinations of two or more amino acid positions in SEQ ID NO: 710 listed in Table 17; and an LCVD that that is ≥90% identical to SEQ ID NO: 711, where the LCVD includes a histidine at any of the specific combinations of two or more amino acid positions in SEQ ID NO: 711 listed in Table 18.

In some examples, the first ABD includes an LCVD that is ≥90% identical to SEQ ID NO: 711, where the LCVD includes a histidine at any of the specific combinations of two or more amino acid positions in SEQ ID NO: 711 listed in Table 18, and/or where the LCVD includes an alanine at position 99 in SEQ ID NO: 711; and an HCVD that that is ≥90% identical to SEQ ID NO: 710, where the HCVD includes a histidine at any of the specific combinations of two or more amino acid positions in SEQ ID NO: 710 listed in Table 17.

In some examples, the first ABD comprises an LCVD comprising SEQ ID NO: 711, and an HCVD that is ≥90% identical to SEQ ID NO: 710, where the HCVD includes a histidine at any of the specific combinations of two or more amino acid positions in SEQ ID NO: 710 listed in Table 17.

In some examples, the first ABD comprises an LCVD that is ≥90% identical to SEQ ID NO: 711, where the LCVD includes a histidine at position 25 in SEQ ID NO: 711; and an HCVD that is ≥90% identical to SEQ ID NO: 710, where the HCVD includes a histidine at any of the specific combinations of two or more amino acid positions in SEQ ID NO: 710 listed in Table 17.

In some examples, the first ABD comprises an LCVD that is ≥90% identical to SEQ ID NO: 711, where the LCVD includes a histidine at position 27 in SEQ ID NO: 711; and an HCVD that is ≥90% identical to SEQ ID NO: 710, where the HCVD includes a histidine at any of the specific combinations of two or more amino acid positions in SEQ ID NO: 710 listed in Table 17.

In some examples, the first ABD comprises an LCVD that is at least 90% identical to SEQ ID NO: 711, where the LCVD includes a histidine at position 29 in SEQ ID NO: 711; and an HCVD that is at least 90% identical to SEQ ID NO: 710, where the HCVD includes a histidine at any of the specific combinations of two or more amino acid positions in SEQ ID NO: 710 listed in Table 17.

In some examples, the first ABD comprises an LCVD that is at least 90% identical to SEQ ID NO: 711, where the LCVD includes an alanine at position 99 in SEQ ID NO: 711; and an HCVD that is at least 90% identical to SEQ ID NO: 710, wherein the HCVD includes a histidine at any of the specific combinations of two or more amino acid positions in SEQ ID NO: 710 listed in Table 17.

In some examples, the first ABD comprises an LCVD that is at least 90% identical to SEQ ID NO: 711, wherein the LCVD includes a histidine at position 30 in SEQ ID NO: 711; and an HCVD that is at least 90% identical to SEQ ID NO: 710, wherein the HCVD includes a histidine at any of the specific combinations of two or more amino acid positions in SEQ ID NO: 710 listed in Table 17.

In some examples, the first ABD comprises an LCVD that is ≥90% identical to SEQ ID NO: 711, wherein the LCVD includes a histidine at position 35 in SEQ ID NO: 711; and an HCVD that is at least 90% identical to SEQ ID NO: 710, where the HCVD includes a histidine at any of the specific combinations of two or more amino acid positions in SEQ ID NO: 710 listed in Table 17.

In some examples, the first ABD comprises an LCVD that is ≥90% identical to SEQ ID NO: 711, wherein the LCVD includes a histidine at position 36 in SEQ ID NO: 711; and an HCVD that is at least 90% identical to SEQ ID NO: 710, where the HCVD includes a histidine at any of the specific combinations of two or more amino acid positions in SEQ ID NO: 710 listed in Table 17.

In some examples, the first ABD comprises an LCVD that is ≥90% identical to SEQ ID NO: 711, wherein the LCVD includes a histidine at position 56 in SEQ ID NO: 711; and an HCVD that is at least 90% identical to SEQ ID NO: 710, where the HCVD includes a histidine at any of the specific combinations of two or more amino acid positions in SEQ ID NO: 710 listed in Table 17.

In some examples, the first ABD comprises an LCVD that is ≥90% identical to SEQ ID NO: 711, wherein the LCVD includes a histidine at position 57 in SEQ ID NO: 711; and an HCVD that is at least 90% identical to SEQ ID NO: 710, where the HCVD includes a histidine at any of the specific combinations of two or more amino acid positions in SEQ ID NO: 710 listed in Table 17.

In some examples, the first ABD comprises an LCVD that is ≥90% identical to SEQ ID NO: 711, wherein the LCVD includes a histidine at position 91 in SEQ ID NO: 711; and an HCVD that is at least 90% identical to SEQ ID NO: 710, where the HCVD includes a histidine at any of the specific combinations of two or more amino acid positions in SEQ ID NO: 710 listed in Table 17.

In some examples, the first ABD comprises an LCVD that is ≥90% identical to SEQ ID NO: 711, wherein the LCVD includes a histidine at position 93 in SEQ ID NO: 711; and an HCVD that is at least 90% identical to SEQ ID NO: 710, where the HCVD includes a histidine at any of the specific combinations of two or more amino acid positions in SEQ ID NO: 710 listed in Table 17.

In some examples, the first ABD comprises an LCVD that is ≥90% identical to SEQ ID NO: 711, wherein the LCVD includes a histidine at position 94 in SEQ ID NO: 711; and an HCVD that is at least 90% identical to SEQ ID NO: 710, where the HCVD includes a histidine at any of the specific combinations of two or more amino acid positions in SEQ ID NO: 710 listed in Table 17.

In some examples, the first ABD comprises an LCVD that is ≥90% identical to SEQ ID NO: 711, wherein the LCVD includes a histidine at position 95 in SEQ ID NO: 711; and an HCVD that is at least 90% identical to SEQ ID NO: 710, where the HCVD includes a histidine at any of the specific combinations of two or more amino acid positions in SEQ ID NO: 710 listed in Table 17.

In some examples, the first ABD comprises an LCVD that is ≥90% identical to SEQ ID NO: 711, wherein the LCVD includes a histidine at position 98 in SEQ ID NO: 711; and an HCVD that is at least 90% identical to SEQ ID NO: 710, where the HCVD includes a histidine at any of the specific combinations of two or more amino acid positions in SEQ ID NO: 710 listed in Table 17.

In some examples, the first ABD comprises an LCVD that is 90% identical to SEQ ID NO: 711, wherein the LCVD includes a histidine at position 99 in SEQ ID NO: 711; and an HCVD that is at least 90% identical to SEQ ID NO: 710, where the HCVD includes a histidine at any of the specific combinations of two or more amino acid positions in SEQ ID NO: 710 listed in Table 17.

In some examples, the first ABD comprises an HCVD that is ≥90% identical to SEQ ID NO: 710, wherein the HCVD includes a histidine at any one of positions 29, 30, 31, 33, 35, 50, 53, 57, 58, 65, 96, 97, 98, 100, 101, 103, 105, 107, 108, 109, 110 and 113 in SEQ ID NO: 710; and an LCVD that is ≥90% identical to SEQ ID NO: 711, where the LCVD includes a histidine at any of the specific combinations of two or more amino acid positions in SEQ ID NO: 711 listed in Table 18.

In some examples, the first ABD comprises an HCVD of SEQ ID NO: 710, or one of 718-767; and/or the first ABD includes an LCVD of SEQ ID NO: 711, or one of 768-802. In some examples of the ABPCs, the first ABD includes an LCVD comprising any one of SEQ ID NO: 711, or one of 768-802 and an HCVD comprising any one of SEQ ID NO: 710, or one of 718-767.

In some examples, the first ABD includes an LCVD comprising SEQ ID NO: 768, and an HCVD comprising one of: SEQ ID NO: 710, or one of 718-767. In some examples, the first ABD includes an LCVD comprising SEQ ID NO: 769, and an HCVD comprising one of: SEQ ID NO: 710, or one of 718-767. In some examples, the first ABD includes an LCVD comprising SEQ ID NO: 770, and an HCVD comprising one of: SEQ ID NO: 710, or one of 718-767. In some examples, the first ABD includes an LCVD comprising SEQ ID NO: 771, and an HCVD comprising one of: SEQ ID NO: 710, or one of 718-767. In some examples, the first ABD includes an LCVD comprising SEQ ID NO: 772, and an HCVD comprising one of: SEQ ID NO: 710, or one of 718-767. In some examples, the first ABD includes an LCVD comprising SEQ ID NO: 773, and an HCVD comprising one of: SEQ ID NO: 710, or one of 718-767. In some examples, the first ABD includes an LCVD comprising SEQ ID NO: 774, and an HCVD comprising one of: SEQ ID NO: 710, or one of 718-767. In some examples, the first ABD includes an LCVD comprising SEQ ID NO: 775, and an HCVD comprising one of: SEQ ID NO: 710, or one of 718-767. In some examples, the first ABD includes an LCVD comprising SEQ ID NO: 776, and an HCVD comprising one of: SEQ ID NO: 710, or one of 718-767. In some examples, the first ABD includes an LCVD comprising SEQ ID NO: 777, and an HCVD comprising one of: SEQ ID NO: 710, or one of 718-767. In some examples, the first ABD includes an LCVD comprising SEQ ID NO: 778, and an HCVD comprising one of: SEQ ID NO: 710, or one of 718-767. In some examples, the first ABD includes an LCVD comprising SEQ ID NO: 779, and an HCVD comprising one of: SEQ ID NO: 710, or one of 718-767. In some examples, the first ABD includes an LCVD comprising SEQ ID NO: 780, and an HCVD comprising one of: SEQ ID NO: 710, or one of 718-767. In some examples, the first ABD includes an LCVD comprising SEQ ID NO: 781, and an HCVD comprising one of: SEQ ID NO: 710, or one of 718-767. In some examples, the first ABD includes an LCVD comprising SEQ ID NO: 782, and an HCVD comprising one of: SEQ ID NO: 710, or one of 718-767. In some examples, the first ABD includes an LCVD comprising SEQ ID NO: 783, and an HCVD comprising one of: SEQ ID NO: 710, or one of 718-767. In some examples, the first ABD includes an LCVD comprising SEQ ID NO: 784, and an HCVD comprising one of: SEQ ID NO: 710, or one of 718-767. In some examples, the first ABD includes an LCVD comprising SEQ ID NO: 785, and an HCVD comprising one of: SEQ ID NO: 710, or one of 718-767. In some examples, the first ABD includes an LCVD comprising SEQ ID NO: 786, and an HCVD comprising one of: SEQ ID NO: 710, or one of 718-767. In some examples, the first ABD includes an LCVD comprising SEQ ID NO: 787, and an HCVD comprising one of: SEQ ID NO: 710, or one of 718-767. In some examples, the first ABD includes an LCVD comprising SEQ ID NO: 788, and an HCVD comprising one of: SEQ ID NO: 710, or one of 718-767. In some examples, the first ABD includes an LCVD comprising SEQ ID NO: 789, and an HCVD comprising one of: SEQ ID NO: 710, or one of 718-767. In some examples, the first ABD includes an LCVD comprising SEQ ID NO: 790, and an HCVD comprising one of: SEQ ID NO: 710, or one of 718-767. In some examples, the first ABD includes an LCVD comprising SEQ ID NO: 791, and an HCVD comprising one of: SEQ ID NO: 710, or one of 718-767. In some examples, the first ABD includes an LCVD comprising SEQ ID NO: 792, and an HCVD comprising one of: SEQ ID NO: 710, or one of 718-767. In some examples, the first ABD includes an LCVD comprising SEQ ID NO: 793, and an HCVD comprising one of: SEQ ID NO: 710, or one of 718-767. In some examples, the first ABD includes an LCVD comprising SEQ ID NO: 794, and an HCVD comprising one of: SEQ ID NO: 710, or one of 718-767. In some examples, the first ABD includes an LCVD comprising SEQ ID NO: 795, and an HCVD comprising one of: SEQ ID NO: 710, or one of 718-767. In some examples, the first ABD includes an LCVD comprising SEQ ID NO: 796, and an HCVD comprising one of: SEQ ID NO: 710, or one of 718-767. In some examples, the first ABD includes an LCVD comprising SEQ ID NO: 797, and an HCVD comprising one of: SEQ ID NO: 710, or one of 718-767. In some examples, the first ABD includes an LCVD comprising SEQ ID NO: 798, and an HCVD comprising one of: SEQ ID NO: 710, or one of 718-767. In some examples, the first ABD includes an LCVD comprising SEQ ID NO: 799, and an HCVD comprising one of: SEQ ID NO: 710, or one of 718-767. In some examples, the first ABD includes an LCVD comprising SEQ ID NO: 800, and an HCVD comprising one of: SEQ ID NO: 710, or one of 718-767. In some examples, the first ABD includes an LCVD comprising SEQ ID NO: 801, and an HCVD comprising one of: SEQ ID NO: 710, or one of 718-767. In some examples, the first ABD includes an LCVD comprising SEQ ID NO: 802, and an HCVD comprising one of: SEQ ID NO: 710, or one of 718-767.

In some examples, the first ABD includes an HCVD of MYT9797 with one or more amino acids substituted with a histidine. In some examples, the first ABD includes an LCVD of MYT9797 with one or more amino acids substituted with a histidine. In some examples, the first ABD includes an HCVD of MYT9797 with one or more amino acids optionally substituted with a histidine; and an LCVD of MYT9797 with one or more amino acids substituted with a histidine. In some examples, the HCVD of MYT9797 comprises SEQ ID NO: 803. In some examples, the LCVD of MYT9797 comprises SEQ ID NO: 804.

In some examples, the first ABD comprises an HCVD comprising a CDR1, a CDR2, and a CDR3 of SEQ ID NOs: 805, 806, and 807, respectively, optionally with collectively a total of one or more amino acid positions in SEQ ID NOs: 805, 806, and 807 substituted with a histidine. In some examples, the first ABD comprises an LCVD comprising a CDR1, a CDR2, and a CDR3 of SEQ ID NOs: 808, 809, and 810, respectively, optionally with collectively a total of one or more amino acid positions in SEQ ID NOs: 808, 809, and 810 substituted with a histidine. In some examples, the first ABD includes: an HCVD comprising a CDR1, a CDR2, and a CDR3 of SEQ ID NOs: 805, 806, and 807, respectively, optionally with collectively a total of one or more amino acid positions in NOs: 805, 806, and 807 optionally substituted with a histidine; and an LCVD comprising a CDR1, a CDR2, and a CDR3 of SEQ ID NOs: 808, 809, and 810, respectively, optionally with collectively a total of one or more amino acid positions in SEQ ID NOs: 808, 809, and 810 substituted with a histidine.

In some examples, the first ABD includes an HCVD that is at least 90% identical to SEQ ID NO: 803, where the HCVD optionally includes a histidine at one or more amino acid positions in SEQ ID NO: 803 selected from: 33, 50, 53, 55-58, 64, 100, and 107. In some examples, the first ABD includes an LCVD that is at least 90% identical to SEQ ID NO: 804, where the LCVD optionally includes a histidine at one or more amino acid positions in SEQ ID NO: 804 selected from: 31, 33-35, 52, 54, 98, and 99. In some examples, the first ABD includes an HCVD that is at least 90% identical to SEQ ID NO: 803, where the HCVD optionally includes a histidine at one or more amino acid positions in SEQ ID NO: 803 selected from: 33, 50, 53, 55-58, 64, 100, and 107, and an LCVD that is at least 90% identical to SEQ ID NO: 804, where the LCVD optionally includes a histidine at one or more amino acid positions in SEQ ID NO: 804 selected from: 31, 33-35, 52, 54, 98, and 99.

In some examples, an HCVD includes an HCVD that is at least 90% identical to SEQ ID NO: 803, where the HCVD includes a histidine at any of the specific combinations of two or more amino acid positions in SEQ ID NO: 803 listed in Table 19.

In some examples, the first ABD includes an HCVD that is ≥90% (e.g., ≥92%, ≥94%, ≥96%, ≥98%, ≥99%, or 100%) identical to SEQ ID NO: 803, where the HCVD includes an alanine at position 55 in SEQ ID NO: 803. In some examples the first ABD includes: an HCVD that is ≥90% (e.g., ≥92%, ≥94%, ≥96%, ≥98%, ≥99%, or 100%) identical to SEQ ID NO: 803, where the HCVD includes an alanine at position 55 in SEQ ID NO: 803, and an LCVD that is ≥90% (e.g., ≥92%, ≥94%, ≥96%, ≥98%, ≥99%, or 100%) identical to SEQ ID NO: 804, where the LCVD optionally includes a histidine at one or more amino acid positions in SEQ ID NO: 804 selected from: 31, 33-35, 52, 54, 98, and 99.

TABLE 19
Exemplary Combinations of Amino Acid Positions in SEQ ID NO: 803
that can be Substituted with Histidine (or Alanine if indicated)

| Col | 1 | 2 | 3 | 4 | 5 | 6 | 7 | 8 | 9 | 10 |
|---|---|---|---|---|---|---|---|---|---|---|
| Pos | 33 | 50 | 53 | 55 | 56 | 57 | 58 | 64 | 100 | 107 |

Exemplary 2, 3, and 4 AA position combinations that can be substituted
with histidine (or an alanine at position 55 of SEQ ID NO: 803)

| | |
|---|---|
| A | Each and every unique 2-position combination of X & Y, wherein X and Y are each |
| | selected (without replacement) from a SEQ ID NO: 803 AA position indicated in |
| | columns 1-10. For example, the combinations of X & Y embodied by SEQ ID NO: |
| | 803 AA position 33, 50, 53, 55-58, 64, 100, and 107 (X) + any one of positions 33, |
| | 50, 53, 55-58, 64, 100, and 107 not selected for X (Y). |
| B | Each and every unique 3-position combination of X & Y & Z, wherein X, Y, and Z |
| | are each selected (without replacement) from a SEQ ID NO: 803 AA position |
| | indicated in columns 1-10. For example, the combinations of X & Y & Z embodied |
| | by SEQ ID NO: 803 AA position 33, 50, 53, 55-58, 64, 100, and 107 (X) + any one of |
| | positions 33, 50, 53, 55-58, 64, 100, and 107 not selected for X (Y) + any one of |
| | positions 33, 50, 53, 55-58, 64, 100, and 107 not selected for X or Y (Z). |
| C | Each and every unique 4-position combination of X & Y & Z & α, wherein X, Y, Z, |
| | and α are each selected (without replacement) from a SEQ ID NO: 803 AA position |
| | indicated in columns 1-10. For example, the combinations of X & Y & Z & α |
| | embodied by SEQ ID NO: 803 AA position 33, 50, 53, 55-58, 64, 100, and 107 (X) + |
| | any one of positions 33, 50, 53, 55-58, 64, 100, and 107 not selected for X (Y) + any |
| | one of positions 33, 50, 53, 55-58, 64, 100, and 107 not selected for X or Y (Z) + any |
| | one of positions 33, 50, 53, 55-58, 64, 100, and 107 not selected for X, Y, or Z (α). |

In some examples, the first ABD includes an LCVD that is ≥90% identical to SEQ ID NO: 804, where the LCVD includes a histidine at any of the specific combinations of two or more amino acid positions in SEQ ID NO: 804 listed in Table 20 and/or where the LCVD includes an alanine at position 34 or 52 of SEQ ID NO: 804.

In some examples, the first ABD includes an LCVD that is 90% identical to SEQ ID NO: 804, where the LCVD includes an alanine at position 34 or 52 in SEQ ID NO: 804.

In some examples the first ABD includes: an LCVD that is ≥90% identical to SEQ ID NO: 804, where the LCVD includes an alanine at position 34 or 52 in SEQ ID NO: 804, and an HCVD that is ≥90% identical to SEQ ID NO: 803, where the HCVD optionally includes a histidine at one or more amino acid positions in SEQ ID NO: 803 selected from: 33, 50, 53, 55-58, 64, 100, and 107.

In some examples, the first ABD includes an LCVD that is ≥90% identical to SEQ ID NO: 804, where the LCVD optionally includes a histidine at one or more amino acid positions in SEQ ID NO: 804 selected from: 31, 33-35, 52, 54, 98, and 99 and/or where the LCVD includes an alanine at position 34 or 52 in SEQ ID NO: 804. In some examples the first ABD includes: an LCVD that is ≥90% identical to SEQ ID NO: 804, where the LCVD optionally includes a histidine at one or more amino acid positions in SEQ ID NO: 804 selected from: 31, 33-35, 52, 54, 98, and 99 and/or where the LCVD includes an alanine at position 34 or 52 in SEQ ID NO: 804, and an HCVD that is ≥90% identical to SEQ ID NO: 803, where the HCVD optionally includes a histidine at one or more amino acid positions in SEQ ID NO: 803 selected from: 33, 50, 53, 55-58, 64, 100, and 107.

In some examples the first ABD includes an LCVD that is ≥90% identical to SEQ ID NO: 804, where the LCVD optionally includes a histidine at one or more amino acid positions in SEQ ID NO 804 selected from: 31, 33-35, 52, 54, 98, and 99 and/or where the LCVD includes an alanine at position 34 or 52 in SEQ ID NO: 804, and an HCVD that is ≥900 identical to SEQ TD NO: 803, where the HCVD optionally includes a histidine at one or more amino acid positions in SEQ TD NO: 803 selected from: 33, 50, 53, 55-58, 64, 100, and 107 and/or where the HCVD includes an alanine at position 55.

TABLE 20
Exemplary Combinations of Amino Acid Positions in SEQ ID NO: 804
that can be Substituted with Histidine (or Alanine if indicated)

| Col | 1 | 2 | 3 | 4 | 5 | 6 | 7 | 8 |
|---|---|---|---|---|---|---|---|---|
| Pos | 31 | 33 | 34 | 35 | 52 | 54 | 98 | 99 |

Exemplary 2, 3, and 4 AA position combinations that can be substituted
with histidine (or an alanine at position 34 or 52 of SEQ ID NO: 804)

| | |
|---|---|
| A | Each and every unique 2-position combination of X & Y, wherein X and Y are each |
| | selected (without replacement) from a SEQ ID NO: 804 AA position indicated in |
| | columns 1-8. For example, the combinations of X & Y embodied by SEQ ID NO: |
| | 804 AA position 31, 33-35, 52, 54, 98, and 99 (X) + any one of positions 31, 33-35, |
| | 52, 54, 98, and 99 not selected for X (Y). |
| B | Each and every unique 3-position combination of X & Y & Z, wherein X, Y, and Z |
| | are each selected (without replacement) from a SEQ ID NO: 804 AA position |
| | indicated in columns 1-8. For example, the combinations of X & Y & Z embodied by |
| | SEQ ID NO: 804 AA position 31, 33-35, 52, 54, 98, and 99 (X) + any one of |
| | positions 31, 33-35, 52, 54, 98, and 99 not selected for X (Y) + any one of positions |
| | 31, 33-35, 52, 54, 98, and 99 not selected for X or Y (Z). |
| C | Each and every unique 4-position combination of X & Y & Z & α, wherein X, Y, Z, |
| | and α are each selected (without replacement) from a SEQ ID NO: 804 AA position |
| | indicated in columns 1-8. For example, the combinations of X & Y & Z & α |
| | embodied by SEQ ID NO: 804 AA position 31, 33-35, 52, 54, 98, and 99 (X) + any |
| | one of positions 31, 33-35, 52, 54, 98, and 99 not selected for X (Y) + any one of |
| | positions 31, 33-35, 52, 54, 98, and 99 not selected for X or Y (Z) + any one of |
| | positions 31, 33-35, 52, 54, 98, and 99 not selected for X, Y, or Z (α). |

In some examples, the first ABD includes an HCVD that is ≥90% identical to SEQ ID NO: 803, where the HCVD includes a histidine at any of the specific combinations of two or more amino acid positions in SEQ ID NO: 803 listed in Table 19; and an LCVD that that is ≥90% identical to SEQ ID NO: 804, where the LCVD includes a histidine at any of the specific combinations of two or more amino acid positions in SEQ ID NO: 804 listed in Table 20.

In some examples, the first ABD includes an LCVD that is ≥90% identical to SEQ ID NO: 804, where the LCVD includes a histidine at any of the specific combinations of two or more amino acid positions in SEQ ID NO: 804 listed in Table 20, and/or where the LCVD includes an alanine at position 34 or 52 in SEQ ID NO: 804; and an HCVD that that is ≥90% identical to SEQ ID NO: 803, where the HCVD includes a histidine at any of the specific combinations of two or more amino acid positions in SEQ ID NO: 803 listed in Table 19.

In some examples, the first ABD comprises an LCVD comprising SEQ ID NO: 804, and an HCVD that is ≥90% identical to SEQ ID NO: 803, where the HCVD includes a histidine at any of the specific combinations of two or more amino acid positions in SEQ ID NO: 803 listed in Table 19.

In some examples, the first ABD comprises an LCVD that is 90% identical to SEQ ID NO: 804, where the LCVD includes a histidine at position 31 in SEQ ID NO: 804; and an HCVD that is ≥90% identical to SEQ ID NO: 803, where the HCVD includes a histidine at any of the specific combinations of two or more amino acid positions in SEQ ID NO: 803 listed in Table 19.

In some examples, the first ABD comprises an LCVD that is ≥90% identical to SEQ ID NO: 804, where the LCVD includes a histidine at position 33 in SEQ ID NO: 804; and an HCVD that is ≥90% identical to SEQ ID NO: 803, where the HCVD includes a histidine at any of the specific combinations of two or more amino acid positions in SEQ ID NO: 803 listed in Table 19.

In some examples, the first ABD comprises an LCVD that is ≥90% identical to SEQ ID NO: 804, where the LCVD includes a histidine at position 34 in SEQ ID NO: 804; and an HCVD that is ≥90% identical to SEQ ID NO: 803, where the HCVD includes a histidine at any of the specific combinations of two or more amino acid positions in SEQ ID NO: 803 listed in Table 19.

In some examples, the first ABD comprises an LCVD that is ≥90% identical to SEQ ID NO: 804, where the LCVD includes a histidine at position 35 in SEQ ID NO: 804; and an HCVD that is ≥90% identical to SEQ ID NO: 803, where the HCVD includes a histidine at any of the specific combinations of two or more amino acid positions in SEQ ID NO: 803 listed in Table 19.

In some examples, the first ABD comprises an LCVD that is 90% identical to SEQ ID NO: 804, where the LCVD includes a histidine at position 52 in SEQ ID NO: 804; and an HCVD that is ≥90% identical to SEQ ID NO: 803, where the HCVD includes a histidine at any of the specific combinations of two or more amino acid positions in SEQ ID NO: 803 listed in Table 19.

In some examples, the first ABD comprises an LCVD that is ≥90% identical to SEQ ID NO: 804, where the LCVD includes a histidine at position 54 in SEQ ID NO: 804; and an HCVD that is ≥90% identical to SEQ ID NO: 803, where the HCVD includes a histidine at any of the specific combinations of two or more amino acid positions in SEQ ID NO: 803 listed in Table 19.

In some examples, the first ABD comprises an LCVD that is ≥90% identical to SEQ ID NO: 804, where the LCVD includes a histidine at position 98 in SEQ ID NO: 804; and an HCVD that is ≥90% identical to SEQ ID NO: 803, where the HCVD includes a histidine at any of the specific combinations of two or more amino acid positions in SEQ ID NO: 803 listed in Table 19.

In some examples, the first ABD comprises an LCVD that is ≥90% identical to SEQ ID NO: 804, where the LCVD includes a histidine at position 99 in SEQ ID NO: 804; and an HCVD that is ≥90% identical to SEQ ID NO: 803, where the HCVD includes a histidine at any of the specific combinations of two or more amino acid positions in SEQ ID NO: 803 listed in Table 19.

In some examples, the first ABD comprises an LCVD that is at least 90% identical to SEQ ID NO: 804, where the LCVD includes an alanine at position 34 or 52 in SEQ ID NO: 804; and an HCVD that is at least 90% identical to SEQ ID NO: 803, wherein the HCVD includes a histidine at any of the specific combinations of two or more amino acid positions in SEQ ID NO: 803 listed in Table 19.

In some examples, the first ABD comprises an HCVD that is ≥90% identical to SEQ ID NO: 803, wherein the HCVD includes a histidine at any one of positions 33, 50, 53, 55-58, 64, 100, and 107 in SEQ ID NO: 803; and an LCVD that is 90% identical to SEQ ID NO: 804, where the LCVD includes a histidine at any of the specific combinations of two or more amino acid positions in SEQ ID NO: 804 listed in Table 20.

In some examples, the first ABD comprises an HCVD of SEQ ID NO: 803, or one of 811-854; and/or the first ABD includes an LCVD of SEQ ID NO: 804, or one of 855-892. In some examples of the ABPCs, the first ABD includes an LCVD comprising any one of SEQ ID NO: 804, or one of 855-892 and an HCVD comprising any one of SEQ ID NO: 803, or one of 811-854.

In some examples, the first ABD includes an LCVD comprising SEQ ID NO: 855, and an HCVD comprising one of: SEQ ID NO: 803, or one of 811-854. In some examples, the first 5 ABD includes an LCVD comprising SEQ ID NO: 856, and an HCVD comprising one of: SEQ ID NO: 803, or one of 811-854. In some examples, the first ABD includes an LCVD comprising SEQ ID NO: 857, and an HCVD comprising one of: SEQ ID NO: 803, or one of 811-854. In some examples, the first ABD includes an LCVD comprising SEQ ID NO: 858, and an HCVD comprising one of: SEQ ID NO: 803, or one of 811-854. In some examples, the first ABD includes an LCVD comprising SEQ ID NO: 859, and an HCVD comprising one of: SEQ ID NO: 803, or one of 811-854. In some examples, the first ABD includes an LCVD comprising SEQ ID NO: 860, and an HCVD comprising one of: SEQ ID NO: 803, or one of 811-854. In some examples, the first ABD includes an LCVD comprising SEQ ID NO: 861, and an HCVD comprising one of: SEQ ID NO: 803, or one of 811-854. In some examples, the first ABD includes an LCVD comprising SEQ ID NO: 862, and an HCVD comprising one of: SEQ ID NO: 803, or one of 811-854. In some examples, the first ABD includes an LCVD comprising SEQ ID NO: 863, and an HCVD comprising one of: SEQ ID NO: 803, or one of 811-854. In some examples, the first ABD includes an LCVD comprising SEQ ID NO: 864, and an HCVD comprising one of: SEQ ID NO: 803, or one of 811-854. In some examples, the first ABD includes an LCVD comprising SEQ ID NO: 865, and an HCVD comprising one of: SEQ ID NO: 803, or one of 811-854. In some examples, the first ABD includes an LCVD comprising SEQ ID NO: 866, and an HCVD comprising one of: SEQ ID NO: 803, or one of 811-854. In some examples, the first ABD includes an LCVD comprising SEQ ID NO: 867, and an HCVD comprising one of: SEQ ID NO: 803, or one of 811-854. In some examples, the first ABD includes an LCVD comprising SEQ ID NO: 868, and an HCVD comprising one of: SEQ ID NO: 803, or one of 811-854. In some examples, the first ABD includes an LCVD comprising SEQ ID NO: 869, and an HCVD comprising one of: SEQ ID NO: 803, or one of 811-854. In some examples, the first ABD includes an LCVD comprising SEQ ID NO: 870, and an HCVD comprising one of: SEQ ID NO: 803, or one of 811-854. In some examples, the first ABD includes an LCVD comprising SEQ ID NO: 871, and an HCVD comprising one of: SEQ ID NO: 803, or one of 811-854. In some examples, the first ABD includes an LCVD comprising SEQ ID NO: 872, and an HCVD comprising one of: SEQ ID NO: 803, or one of 811-854. In some examples, the first ABD includes an LCVD comprising SEQ ID NO: 873, and an HCVD comprising one of: SEQ ID NO: 803, or one of 811-854. In some examples, the first ABD includes an LCVD comprising SEQ ID NO: 874, and an HCVD comprising one of: SEQ ID NO: 803, or one of 811-854. In some examples, the first ABD includes an LCVD comprising SEQ ID NO: 875, and an HCVD comprising one of: SEQ ID NO: 803, or one of 811-854. In some examples, the first ABD includes an LCVD comprising SEQ ID NO: 876, and an HCVD comprising one of: SEQ ID NO: 803, or one of 811-854. In some examples, the first ABD includes an LCVD comprising SEQ ID NO: 877, and an HCVD comprising one of: SEQ ID NO: 803, or one of 811-854. In some examples, the first ABD includes an LCVD comprising SEQ ID NO: 878, and an HCVD comprising one of: SEQ ID NO: 803, or one of 811-854. In some examples, the first ABD includes an LCVD comprising SEQ ID NO: 879, and an HCVD comprising one of: SEQ ID NO: 803, or one of 811-854. In some examples, the first ABD includes an LCVD comprising SEQ ID NO: 880, and an HCVD comprising one of: SEQ ID NO: 803, or one of 811-854. In some examples, the first ABD includes an LCVD comprising SEQ ID NO: 881, and an HCVD comprising one of: SEQ ID NO: 803, or one of 811-854. In some examples, the first ABD includes an LCVD comprising SEQ ID NO: 882, and an HCVD comprising one of: SEQ ID NO: 803, or one of 811-854. In some examples, the first ABD includes an LCVD comprising SEQ ID NO: 883, and an HCVD comprising one of: SEQ ID NO: 803, or one of 811-854. In some examples, the first ABD includes an LCVD comprising SEQ ID NO: 884, and an HCVD comprising one of: SEQ ID NO: 803, or one of 811-854. In some examples, the first ABD includes an LCVD comprising SEQ ID NO: 885, and an HCVD comprising one of: SEQ ID NO: 803, or one of 811-854. In some examples, the first ABD includes an LCVD comprising SEQ ID NO: 886, and an HCVD comprising one of: SEQ ID NO: 803, or one of 811-854. In some examples, the first ABD includes an LCVD comprising SEQ ID NO: 887, and an HCVD comprising one of: SEQ ID NO: 803, or one of 811-854. In some examples, the first ABD includes an LCVD comprising SEQ ID NO: 888, and an HCVD comprising one of: SEQ ID NO: 803, or one of 811-854. In some examples, the first ABD includes an LCVD comprising SEQ ID NO: 889, and an HCVD comprising one of: SEQ ID NO: 803, or one of 811-854. In some examples, the first ABD includes an LCVD comprising SEQ ID NO: 890, and an HCVD comprising one of: SEQ ID NO: 803, or one of 811-854. In some examples, the first ABD includes an LCVD comprising SEQ ID NO: 891, and an HCVD comprising one of: SEQ ID NO: 803, or one of 811-854. In some examples, the first ABD includes an LCVD comprising SEQ ID NO: 892, and an HCVD comprising one of: SEQ ID NO: 803, or one of 811-854.

Also provided herein are pharmaceutical compositions including any of the ABPCs described herein. Also provided herein are methods of treating a subject in need thereof that include administering a therapeutically effective amount of any of the ABPCs described herein to the subject.

In some examples, a composition including the ABPC (e.g., any of the ABPCs described herein) can provide for an increase (e.g., a detectable increase) (e.g., any of the percents increase or ranges of percents increase recited in US 2022/0281984) in toxin liberation in the target mammalian cell (e.g., any of the target mammalian cells described herein) as compared to a composition including the same amount of a control ABPC (e.g., any of the exemplary control ABPCs described herein).

In some examples, a composition including the ABPC (e.g., any of the ABPCs described herein) can provide for an increase (e.g., a detectable increase) (e.g., any of the folds increase or ranges of folds increase recited in the published application US 2022/0281984) in toxin liberation in the target mammalian cell (e.g., any of the target mammalian cells described herein) as compared to a composition including the same amount of a control ABPC (e.g., any of the exemplary control ABPCs described herein).

In some examples, a composition including the ABPC (e.g., any of the ABPCs described herein) can provide for an increase (e.g., a detectable increase) (e.g., at least a 1%, 2%, 5%, 10%, 15%, 20%, 25%, 30%, 35%, 40%, 45%, 50%, 55%, 60%, 65%, 70%7, 75%, 80%, 85%, 90%, 95%, 100%, 120%, 140%, 160%, 180%, 200%, 250%, 300%, 350%, 400%, 450%, 500%, 1,000%, 2,000%, 3,000%, 4,000%, 5,000%, 6,000%, 7,000%, 8,000%, 9,000%, or a 10,000% increase, or about a 1% to 10,000% increase (e.g., or any of the subranges)) in target mammalian cell killing as compared to a composition including the same amount of a control ABPC.

In some examples, a composition including the ABPC (e.g., any of the ABPCs described herein) can provide for an increase (e.g., a detectable increase) (e.g., at least a 0.1-, 0.2-, 0.3-, 0.4-, 0.5-, 0.6-, 0.7-, 0.8-, 0.9-, 1.0-, 1.2-, 1.4-, 1.5-, 1.6-, 1.8-, 2.0-, 2.2-, 2.4-, 2.5-, 2.6-, 2.8-, 3.0-, 3.5-, 4.0-, 4.5-, 5.0-, 5.5-, 6.0-, 6.5-, 7.0-, 7.5-, 8.0-, 8.5-, 9.0-, 9.5-, 10-, 15-, 20-, 25-, 30-, 35-, 40-, 40-, 45-, 50-, 55-, 60-, 65-, 70-, 80-, 85-, 90-, 95-fold increase, or at least a 100-fold increase, or a 0.1-100-fold increase in target mammalian cell killing as compared to a composition including the same amount of a control ABPC.

In some examples, a composition including any of the ABPCs described herein (e.g., upon contacting target mammalian cells presenting PTK7 on their surface) results in decreased (e.g., ≥a 1%, 5%, 10%, 15%, 20%, 25%, 30%, 35%, 40%, 45%, 50%, 55%, 60%, 65%, 70%, 75%, 80%, 85%, 90%, 95% decrease, or at least a 99% decrease, about a 1%-99% decrease, or any of the subranges of this range described herein) IC₅₀ (for target mammalian cell killing) as compared to the IC₅₀ for a composition including the same amount of a control ABPC.

In some examples, a composition including any of the ABPCs described herein (e.g., upon contacting target mammalian cells presenting PTK7 on their surface) can provide for an increase (e.g., at least a 0.1-, 0.2-, 0.4-, 0.6-, 0.8-, 1-, 2-, 5-, 10-, 15-, 20-, 25-, 30-, 35-, 40-, 45-, 50-, 55-, 60-, 65-, 70-, 75-, 80-, 85-, 90-, 95-fold increase, or at least a 100-fold increase, or about a 0.1-500-fold increase (or any of the subranges of this range described herein) in the ratio of K_{D} on target mammalian cells presenting PTK7 on their surface at a neutral pH to IC₅₀ at the neutral pH on the same target cells, e.g., as compared to a control ABPC.

In some examples, a composition including the ABPC (e.g., any of the ABPCs described herein) can provide for an increase (e.g., a detectable increase) (e.g., at least a 1%, 2%, 5%, 10%, 15%, 20%, 25%, 30%, 35%, 40%, 45%, 50%, 55%, 60%, 65%, 70%, 75%, 80%, 85%, 90%, 95%, 100%, 120%, 140%, 160%, 180%, 200%, 250%, 300%, 350%, 400%, 450%, 500%, 1,000%, 2,000%, 3,000%, 4,000%, 5,000%, 6,000%, 7,000%, 8,000%, 9,000% increase, or at least a 10,000% increase, or about a 1%-10,000% increase (e.g., or any of the subranges of this range described herein)) in endolysosomal delivery in the target mammalian cell as compared to a composition including the same amount of a control ABPC.

In some examples, a composition including the ABPC can provide for an increase (e.g., ≥a 0.1-, 0.2-, 0.3-, 0.4-, 0.5-, 0.6-, 0.7-, 0.8-, 0.9-, 1.0-, 1.2-, 1.4-, 1.5-, 1.6-, 1.8-, 2.0-, 2.2-, 2.4-, 2.5-, 2.6-, 2.8-, 3.0-, 3.5-, 4.0-, 4.5-, 5.0-, 5.5-, 6.0-, 6.5-, 7.0-, 7.5-, 8.0-, 8.5-, 9.0-, 9.5-, 10-, 15-, 20-, 25-, 30-, 35-, 40-, 45-, 50-, 55-, 60-, 65-, 70-, 75-, 80-, 85-, 90-, 95-fold increase, or at least a 100-fold increase, or about a 0.1-100-fold increase in endolysosomal delivery in the target mammalian cell as compared to a composition including the same amount of a control ABPC.

In examples of any of the ABPCs described herein, the target mammalian cell does not express an FcRn receptor, or expresses a lower (e.g., a detectably lower) level (e.g., at least a 1% decreased, at least a 2%, 5%, 10% decrease, at least a 15%, 20%, 25%, 30%, 35%, 40%, 45%, 50%, 55%, 60%, 65%, 70%, 75%, 80%, 85%, 90%, 95% decreased, or at least a 99% decreased level) of FcRn receptor as compared to a FcRn expressing control cell (e.g., HUVEC—ThermoFisher #C0035C). In some examples, the target mammalian cell is a cancer cell. In some examples, the ABPC is cytotoxic or cytostatic to the target mammalian cell.

In some examples, a composition including any of the ABPCs described herein (e.g., upon administration to a subject) results in less (e.g., a 1% decrease to about a 99% decrease, or any of the subranges of this range described herein) of a reduction in the level of PTK7 presented on the surface of the target cell as compared to a composition including the same amount of a control ABPC (e.g., any of the control ABPCs described herein). In some examples, the composition does not result in a detectable reduction in the level of the PTK7 presented on the surface of the target mammalian cell.

In some examples, the ABPC is cross-reactive with a non-human primate PTK7 and a human PTK7. In some examples, the ABPC is cross-reactive with a non-human primate PTK7, a human PTK7, and one or both of rat PTK7 and a mouse PTK7. In some examples, the ABPC is cross-reactive with a non-human primate PTK7, a human PTK7, a rat PTK7, and a mouse PTK7. In some examples, the ABPC is cross-reactive with mouse PTK7 and rat PTK7. In some examples, the ABD binds to an epitope of PTK7 that is present on the surface of cells from an Old World Monkey.

Some examples of any of the ABPCs described herein can further include a second ABD (e.g., any of the exemplary ABDs described herein). Non-limiting aspects of these methods are described below, and can be used in any combination without limitation. Additional aspects of these methods are known in the art.

### PTK7 or Epitope of PTK7

Multiple PTK7-binding monoclonal antibodies have been described in the literature and can be used as a template for engineering pH-dependent binding. The amino acid sequence of the full-length Human PTK7 can be found in SEQ ID NO: 298 and the sequence of the mature Human PTK7 can be found in SEQ ID NO: 299. The amino acid sequence of the extracellular domain of PTK7 can be found in SEQ ID NO: 300.

### Antigen-Binding Protein Constructs

Any of the antigen-binding protein constructs (ABPCs) described herein can be a single polypeptide, or can include 2, 3, 4, 5, 6, 7, 8, 9, or 10 (the same or different) polypeptides. In some embodiments where the ABPC is a single polypeptide, the ABPC can include a single ABD or two ABDs. In some embodiments where the ABPC is a single polypeptide and includes two ABDs, the first and second ABDs can be identical or different from each other (and can specifically bind to the same or different antigens or epitopes).

In some embodiments where the ABPC is a single polypeptide, the first ABD and the second ABD (if present) can each be independently selected from the group of: a VH domain, a VHH domain, a VNAR domain, and a scFv. In some embodiments where the ABPC is a single polypeptide, the antigen-binding protein construct can be a BiTe, a (scFv)₂, a nanobody, a nanobody-HSA, a DART, a TandAb, a scDiabody, a scDiabody-CH3, scFv-CH-CL-scFv, a HSAbody, scDiabody-HAS, a tandem-scFv, an Adnectin, a DARPin, a fibronectin, and a DEP conjugate. Additional examples of ABDs that can be used when the ABPC is a single polypeptide are known in the art.

A V_{H}H domain is a single monomeric variable antibody domain that can be found in camelids. A V_{NAR} domain is a single monomeric variable antibody domain that can be found in cartilaginous fish. Non-limiting aspects of V_{H}H domains and V_{NAR} domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments where the ABPC is a single polypeptide and includes two ABDs, the first ABD and the second ABD can both be VHH domains, or at least one ABD can be a VHH domain. In some embodiments where the ABPC is a single polypeptide and includes two ABDs, the first ABD and the second ABD are both V_{NAR} domains, or at least one ABD is a V_{NAR} domain. In some embodiments where the ABPC is a single polypeptide, the first ABD is a scFv domain. In some embodiments where the ABPC is a single polypeptide and includes two ABDs, the first ABD and the second ABD can both be scFv domains, or at least one ABD can be a scFv domain.

In some embodiments, the ABPC can include two or more polypeptides (e.g., 2, 3, 4, 5, 6, 7, 8, 9, or 10 polypeptides). In some embodiments where the ABPC includes two or more polypeptides, two, three, four, five or six of the polypeptides of the two or more polypeptides can be identical.

In some embodiments where the ABPC includes two or more polypeptides (e.g., 2, 3, 4, 5, 6, 7, 8, 9, or 10 polypeptides), two or more of the polypeptides of the ABPC can assemble (e.g., non-covalently assemble) to form one or more ABDs, e.g., an antigen-binding fragment of an antibody (e.g., any of the antigen-binding fragments of an antibody described herein), a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a Kk-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, and a DEP conjugate. See, e.g., Spiess et al., Mol. Immunol. 67:95-106, 2015, incorporated in its entirety herewith, for a description of these elements. Non-limiting examples of an antigen-binding fragment of an antibody include an Fv fragment, a Fab fragment, a F(ab′)₂ fragment, and a Fab′ fragment. Additional examples of an antigen-binding fragment of an antibody is an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

A “Fv” fragment includes a non-covalently-linked dimer of one HCVD and one LCVD.

A “Fab” fragment includes, the constant domain of the light chain and the first constant domain (CHI) of the heavy chain, in addition to the heavy and LCVDs of the Fv fragment.

A “F(ab′)₂” fragment includes two Fab fragments joined, near the hinge region, by disulfide bonds.

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

DARTs are described in, e.g., Garber, Nature Reviews Drug Discovery 13:799-801, 2014.

Additional aspects of ABPCs are known in the art.

### Antigen-Binding Domains

In some embodiments of any of the antigen-binding protein constructs (ABPCs) described herein, the dissociation rate of the first ABD (and optionally the second ABD, if present) at a pH of about 4.0 to about 6.5 (e.g., about 4.0 to about 6.4, or any of the ranges of pH recited in US 2022/0281984, which is incorporated by reference herein in its entirety) is faster (e.g., at least 5% faster, or any of the % faster or ranges of % faster recited in US 2022/0281984), than the dissociation rate at a pH of about 7.0 to about 8.0 (e.g., about 7.0-7.9, 7.0-7.8, 7.0-7.7, 7.0-7.6, 7.0-7.5, 7.0-7.4, 7.0-7.3, 7.0-7.2, 7.0-7.1, 7.1-8.0, 7.1-7.9, 7.1-7.8, 7.1-7.7, 7.1-7.6, 7.1-7.5, 7.1-7.4, 7.1-7.3, 7.1-7.2, 7.2-8.0, 7.2-7.9, 7.2-7.8, 7.2-7.7, 7.2-7.6, 7.2-7.5, 7.2-7.4, 7.2-7.3, 7.3-8.0, 7.3-7.9, 7.3-7.8, 7.3-7.7, 7.3-7.6, 7.3-7.5, 7.3-7.4, 7.4-8.0, 7.4-7.9, 7.4-7.8, 7.4-7.7, 7.4-7.6, 7.4-7.5, 7.5-8.0, 7.5-7.9, 7.5-7.8, 7.5-7.7, 7.5-7.6, 7.5-8.0, 7.6-7.9, 7.6-7.8, 7.6-7.7, 7.7-8.0, 7.7-7.9, 7.7-7.8, 7.8-8.0, 7.8-7.9, or about 7.9-8.0).

In some embodiments of any of the ABPCs described herein, the dissociation constant (K_{D}) of the first ABD (and optionally the second ABD, if present) at a pH of about 4.0 to about 6.5 (e.g., any of the subranges of this range described herein) is greater (e.g., detectably greater) (e.g., at least about 5, 10, 15, 20, 25, 30, 35, 40, 45, 50, 55, 60, 65, 70, 80, 85, 90, 95, 100, 120, 140, 160, 180, 200, 220, 240, 260, 280, 300, 320, 340, 360, 380, 400, 420, 440, 460, 480, 500, 1,000, 2,000, 3,000, 4,000, 5,000, 6,000, 7,000, 8,000, 9,000, or at least about 10,000% greater, or about 5% to about 10,000% greater, or any of the ranges of percents greater K_{D} recited in US 2022/0281984), which are incorporated by reference herein in their entirety) than the K_{D} at a pH of about 7.0 to about 8.0 (e.g., any of the subranges of this range described herein).

In some embodiments of any of the antigen-binding protein constructs (ABPCs) described herein, the dissociation rate of the first ABD (and optionally the second ABD, if present) at a pH of about 4.0 to about 6.5 (e.g., any of the subranges of this range described herein) is faster (e.g., at least 0.2-fold faster, or any of the folds faster or ranges of folds faster recited in US 2022/0281984 than the dissociation rate at a pH of ~7.0 to ~8.0 (e.g., or any of the subranges).

In some embodiments of any of the antigen-binding protein constructs (ABPCs) described herein, the dissociation constant (K_{D}) of the first ABD (and optionally the second ABD, if present) at a pH of about 4.0 to about 6.5 (e.g., any of the subranges of this range described herein) is greater (e.g., detectably greater) (e.g., at least 0.2-fold greater, or any of the fold greater or ranges of fold greater recited in US 2022/0281984, than the K_{D} at a pH of about 7.0 to about 8.0 (e.g., any of the subranges of this range described herein).

In some embodiments of the ABPCs that include a first ABD and a second ABD, the first and second ABDs are identical or are at least 80% identical (e.g., ≥82%, ≥84%, ≥86%, ≥88%, ≥90%, ≥91%, ≥92%, ≥93%, ≥94%, ≥95%, ≥96%, ≥97%, ≥98%, or ≥99% identical) in amino acid sequence to each other. In some embodiments, the ABPCs that include a first ABD and a second ABD, the first ABD and the second ABD have a sequence that is less than 80% identical (e.g., <75%, <70%, <65%, <60%, <55%, <50%, <45%, <40%, <35%, <30%, <25%, <20%, <15%, <10%, or <5% identical) to each other. In some embodiments of ABPCs that include a first and a second ABD, the first and second ABD binds two different epitopes (e.g., two different epitopes on PTK7 or the first ABD binding specifically to PTK7 and the second ABD binding to an antigen other than PTK7).

In some embodiments of any of the ABPCs described herein, the K_{D} of the first ABD (and optionally, the second ABD if present) at a pH of about 7.0-8.0 (e.g., any of the subranges of this range described herein) is between about 1 pM-5 μM (e.g., 1 pM-2 μM, or any of the ranges of K_{D} recited in US 2022/0281984.

In some embodiments of any of the ABPCs described herein, the K_{D} of the first ABD (and optionally, the second ABD, if present) at a pH of about 4.0-6.5 (e.g., any of the subranges of this range described herein) can be greater than 1 nM (e.g., between about 1 nM-1 mM, or any of the ranges of K_{D} recited in US 2022/0281984.

A variety of different methods known in the art can be used to determine the K_{D} values of any of the antigen-binding protein constructs described herein (e.g., an electrophoretic mobility shift assay, a filter binding assay, surface plasmon resonance, a biomolecular binding kinetics assay, in vitro binding assay on antigen-expressing cells, etc.).

In some examples, the half-life of the ABPC in vivo is decreased (e.g., a detectable decrease) (e.g., at least a 1% decrease, or any of the percents decrease or ranges of percents decrease recited in US 2022/0281984, as compared to the half-life of a control ABPC (e.g., any of the exemplary control ABPCs described herein). Conversely, in some examples, the half-life of the ABPC in vivo may be increased as compared to the half-life of a control ABPC.

### Conjugation

In some embodiments, the ABPCs provided herein can be conjugated to a drug (e.g., a chemotherapeutic drug, a small molecule), a toxin, or a radioisotope. Non-limiting examples of drugs, toxins, and radioisotopes (e.g., useful for the treatment of cancer) are known in the art.

In some embodiments, at least one polypeptide of any of the ABPCs described herein is conjugated to the toxin, the radioisotope, or the drug via a cleavable linker. In some embodiments, the cleavable linker includes a protease cleavage site. In some embodiments, the cleavable linker is cleaved on the ABPC once it is transported to the lysosome or late endosome by the target mammalian cell. In some embodiments, cleavage of the linker functionally activates the drug or toxin.

In some embodiments, at least one polypeptide of any of the ABPCs described herein is conjugated to the toxin, the radioisotope, or the drug via a non-cleavable linker. In some embodiments, the conjugated toxin, radioisotope, or drug is released during lysosomal and/or late endosomal degradation of the ABPC.

Non-limiting examples of cleavable linkers include: hydrazone linkers, peptide linkers, disulfide linkers, and thioether linkers. See, e.g., Carter et al., Cancer J. 14(3):154-169, 2008; Sanderson et al., Clin. Cancer Res. 11(2 Pt1):843-852, 2005; Chari et al., Acc. Chem. Res. 41(1):98-107, 2008; Oflazoglu et al., Clin. Cancer Res. 14(19): 6171-6180, 2008; and Lu et al., Int. J Mol. Sci. 17(4): 561, 2016.

Non-limiting examples of non-cleavable linkers include: maleimide alkane-linkers and maleimide cyclohexane linker (MMC) (see, e.g., McCombs et al., AAPS J. 17(2):339-351, 2015).

In some embodiments, any of the ABPCs described herein is cytotoxic or cytostatic to the target mammalian cell. In some embodiments, the antibodies provided herein can comprise one or more amino acid substitutions to provide a conjugation site (e.g., conjugated to a drug, a toxin, a radioisotope). In some embodiments, the antibodies provided herein can have one conjugation site. In some embodiments, the antibodies described herein can have two conjugation sites. In some embodiments, the antibodies provided herein can have three or more conjugation sites.

Naturally-occurring cysteine amino acids can also provide conjugation. In some embodiments, the antibodies provided herein can have a drug, a toxin, or a radioisotope conjugated at one or more naturally-occurring conjugation sites. In some embodiments, the antibodies provided herein can have a drug, a toxin, or a radioisotope conjugated at one or more naturally occurring conjugation sites. In some embodiments, the antibodies provided herein can have a drug, a toxin, or a radioisotope conjugated at one or more naturally occurring conjugation sites and one or more engineered conjugation sites.

Conjugation through engineered cysteines is achieved by methods known in the art. Briefly, engineered cysteine-containing antibody is prepared for conjugation by treatment with a reducing agent, for example, tris (2-carboxyethyl) phosphine (TCEP), Dithiothreitol (DTT), or 2-Mercaptoethanol (BME). In the reduction reaction the reducing reagent with disulfide bonds in the antibody, breaking interchain disulfides and removing disulfide caps from the engineered cysteines. An optional reoxidation step, achieved by exposure of the solution to air, or an oxidizing agent such as dehydroascorbic acid, allows reformation of the interchain disulfide bonds, leaving the engineered cysteines with a thiolate reactive group. Conjugation with a maleimide functionality on the linker-payload, maleimide-vc-MMAE, is achieved by reaction with the payload in buffered solution, containing cosolvent such as ethanol, dimethylacetamide (DMA), or dimethyl sulfoxide (DMSO). The crude conjugated antibody solution is purified by size exclusion chromatography, or selective filtration methods, such as tangential flow filtration. In this step, residual unreacted payload, reducing agent and oxidizing agents are removed from the reaction mixture, and the conjugated ADC product may be transferred into a desirable formulation buffer.

Conjugation through hinge cysteines is achieved by similar methods, using antibodies with, or without, additional engineered cysteine conjugation sites. Briefly, the antibody is prepared for conjugation by treatment with a reducing agent, for example, tris (2-carboxyethyl) phosphine (TCEP) or Dithiothreitol (DTT). The reducing strength and concentration of the reducing agent are selected such that some or all of the interchain disulfide bonds are reduced leaving free cysteines for conjugation. The solution may be directly conjugated in the presence of excess reducing agent. Conjugation with a maleimide functionality on the linker-payload, maleimide-vc-MMAE, is achieved by reaction with the payload in buffered solution, containing cosolvent such as ethanol, dimethylacetamide (DMA), or dimethyl sulfoxide (DMSO). Unreacted linker-payload may be rendered non-reactive by addition of a sacrificial thiolate molecule such as acetyl-cysteine. The crude conjugated antibody solution may be further purified by methods known in the art, including hydrophobic interaction chromatography, ion-exchange chromatography, or mixed-mode chromatography such as ceramic hydroxyapatite chromatography. Isolation of chromatography fractions allows selection of the desired antibody to payload ratio and removal of unreacted antibody, protein aggregates and fragments, and payload-related reaction side products. The purified antibody drug conjugate may be further purified by size exclusion chromatography, or selective filtration methods, such as TFF. In this step the conjugated ADC product may also be transferred into a desirable formulation buffer.

In some examples, an antibody conjugate can be made comprising an antibody linked to monomethyl auristatin E (MMAE) via a valine-citrulline (vc) linker (hereafter, PTK7-IgG-DC). Conjugation of the antibody with vcMMAE begins with a partial reduction of the PTK7-IgG followed by reaction with maleimidocaproyl-Val-Cit-PABC-MMAE (vcMMAE). The PTK7-IgG (10 mg/mL) is partially reduced by addition of TCEP (molar equivalents of TCEP:mAb is 2:1) followed by incubation at 4° C. overnight. The reduction reaction is then warmed to 25° C. To conjugate all of the thiols, vcMMAE is added to a final vcMMAE:reduced Cys molar ratio of 1:10. The conjugation reaction is carried out in the presence of 10% v/v of DMA and allowed to proceed at 25° C. for 60 minutes.

In some examples, an antibody conjugate (ADC) is made comprising the PTK7-binding IgG (hereafter, PTK7-IgG) described herein linked to monomethyl auristatin E (MMAE) via a valine-citrulline (vc) linker (hereafter, PTK7-IgG-DC). Conjugation of the antibody with vcMMAE begins with a partial reduction of the PTK7-IgG followed by reaction with maleimidocaproyl-Val-Cit-PABC-MMAE (vcMMAE). The PTK7-IgG (10 mg/mL) is reduced by addition of DTT (molar equivalents of DTT:mAb is 100:1) followed by incubation at 25° C. overnight. The reduced PTK7-IgG (10 mg/mL) is then re-oxidized by exposure to DHAA (molar equivalents of DHAA:mAb is 10:1) followed by incubation at 25° C. for 2 hours. To conjugate all of the thiols, vcMMAE is added to a final vcMMAE:mAb molar ratio of 4:1. The conjugation reaction is carried out in the presence of 10% v/v of DMA and allowed to proceed at 25° C. for 3 hours.

### Expression of an Antigen-Binding Protein Construct in a Cell

Also provided herein are methods of generating a recombinant cell that expresses an ABPC (e.g., any of the ABPCs described herein) that include: introducing into a cell a nucleic acid encoding the ABPC to produce a recombinant cell; and culturing the recombinant cell under conditions sufficient for the expression of the ABPC. In some embodiments, the introducing step includes introducing into a cell an expression vector including a nucleic acid encoding the ABPC to produce a recombinant cell.

Any of the ABPCs described herein can be produced by any cell, e.g., a eukaryotic cell or a prokaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. As used herein, the term “prokaryotic cell” refers to a cell that does not have a distinct, membrane-bound nucleus. In some embodiments, the prokaryotic cell is a bacterial cell.

Methods of culturing cells are well known in the art. Cells can be maintained in vitro under conditions that favor proliferation, differentiation, and growth. Briefly, cells can be cultured by contacting a cell (e.g., any cell) with a cell culture medium that includes the necessary growth factors and supplements to support cell viability and growth.

Methods of introducing nucleic acids and expression vectors into a cell (e.g., a eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalection, hydrodynamic delivery, magnetofection, viral transduction (e.g., adenoviral and lentiviral transduction), and nanoparticle transfection.

Provided herein are methods that further include isolation of the ABPCs from a cell (e.g., a eukaryotic cell) using techniques well-known in the art (e.g., ammonium sulfate precipitation, polyethylene glycol precipitation, ion-exchange chromatography (anion or cation), chromatography based on hydrophobic interaction, metal-affinity chromatography, ligand-affinity chromatography, and size exclusion chromatography).

### Methods of Treatment

Provided herein are methods of treating a cancer characterized by having a population of cancer cells that have PTK7 or an epitope of PTK7 presented on their surface, which include: administering a therapeutically effective amount of any of the pharmaceutical compositions described herein or any of the ABPCs described herein to a subject identified as having a cancer characterized by having the population of cancer cells.

Also provided herein are methods of reducing the volume of a tumor in a subject, wherein the tumor is characterized by having a population of cancer cells that have PTK7 or an epitope of PTK7 presented on their surface, that include: administering a therapeutically effective amount of any of the pharmaceutical compositions described herein or any of the ABPCs described herein to a subject identified as having a cancer characterized by having the population of cancer cells. In some embodiments of any of the methods described herein, the volume of at least one tumor (e.g., solid tumor) or tumor location (e.g., a site of metastasis) is reduced (e.g., a detectable reduction) by ≥1%, ≥2%, ≥3%, ≥4%, ≥5%, ≥6%, ≥8%, ≥10%, ≥12%, ≥14%, ≥16%, ≥18%, ≥20%, ≥22%, ≥24%, ≥26%, ≥28%, ≥30%, ≥35%, ≥40%, ≥45%, ≥50%, ≥55%, ≥60%, ≥65%, ≥70%, ≥75%, ≥80%, ≥85%, ≥90%, ≥95%, or ≥99%) reduced as compared to the size of the at least one tumor (e.g., solid tumor) before administration of the ABPC.

Also provided herein are methods of inducing cell death in a cancer cell in a subject, wherein the cancer cell has PTK7 or an epitope of PTK7 presented on its surface, that include: administering a therapeutically effective amount of any of the pharmaceutical compositions of described herein or any of the ABPCs described herein to a subject identified as having a cancer characterized as having the population of cancer cells. In some embodiments, the cell death that is induced is necrosis. In some embodiments, the induced cell death is apoptosis.

In some embodiments of any of the methods described herein, the cancer is a primary tumor or a metastasis.

In some embodiments of any of the methods described herein, the cancer is a non-T-cell-infiltrating tumor. In some embodiments of any of the methods described herein, the cancer is a T-cell-infiltrating tumor.

Provided herein are methods of decreasing the risk of developing a metastasis or decreasing the risk of developing an additional metastasis in a subject having a cancer, wherein the cancer is characterized by having a population of cancer cells that have PTK7 or an epitope of PTK7 presented on their surface, that include: administering a therapeutically effective amount of any of the pharmaceutical compositions of described herein or any of the ABPCs described herein to a subject identified as having a cancer characterized as having the population of cancer cells. In some embodiments, the risk of developing a metastasis or the risk of developing an additional metastasis is decreased by ≥1%, by ≥2%, ≥3%, ≥4%, ≥5%, ≥6%, ≥8%, ≥10%, ≥12%, ≥14%, ≥16%, ≥18%, ≥20%, ≥25%, ≥30%, ≥35%, ≥40%, ≥45%, ≥50%, ≥55%, ≥60%, ≥65%, ≥70%, ≥75%, ≥80%, ≥85%, ≥90%, ≥95%, or ≥99% in the subject as compared to the risk of a subject having a similar cancer, but administered no treatment or a treatment that does not include the administration of any of the ABPCs described herein.

In some embodiments of any of the methods described herein, the cancer is a non-T-cell-infiltrating tumor. In some embodiments of any of the methods described herein, the cancer is a T-cell-infiltrating tumor. In some embodiments of any of the methods described herein, the cellular compartment is part of the endosomal/lysosomal pathway. In some embodiments of any of the methods described herein, the cellular compartment is an endosome.

The term “subject” refers to any mammal. In some embodiments, the subject or “subject suitable for treatment” may be a canine (e.g., a dog), feline (e.g., a cat), equine (e.g., a horse), ovine, bovine, porcine, caprine, primate, e.g., a simian (e.g., a monkey (e.g., marmoset, baboon), or an ape (e.g., a gorilla, chimpanzee, orangutan, or gibbon) or a human; or rodent (e.g., a mouse, a guinea pig, a hamster, or a rat). In some embodiments, the subject or “subject suitable for treatment” may be a non-human mammal, especially mammals that are conventionally used as models for demonstrating therapeutic efficacy in humans (e.g., murine, lapine, porcine, canine or primate animals) may be employed.

As used herein, treating includes reducing the number, frequency, or severity of one or more (e.g., two, three, four, or five) signs or symptoms of a cancer in a patient having a cancer (e.g., any of the cancers described herein). For example, treatment can reduce cancer progression, reduce the severity of a cancer, or reduce the risk of re-occurrence of a cancer in a subject having the cancer.

Provided herein are methods of inhibiting the growth of a solid tumor in a subject (e.g., any of the subjects described herein) that include administering to the subject a therapeutically effective amount of any of the ABPCs described herein or any of the pharmaceutical compositions described herein (e.g., as compared to the growth of the solid tumor in the subject prior to treatment or the growth of a similar solid tumor in a different subject receiving a different treatment or receiving no treatment).

In some embodiments of any of the methods described herein, the growth of a solid tumor is primary growth of a solid tumor. In some embodiments of any of the methods described herein, the growth of a solid tumor is recurrent growth of a solid tumor. In some embodiments of any of the methods described herein, the growth of a solid tumor is metastatic growth of a solid tumor. In some embodiments, treatment results in about a 1% decrease to about 99% decrease (or any of the subranges of this range described herein) in the growth of a solid tumor in the subject (e.g., as compared to the growth of the solid tumor in the subject prior to treatment or the growth of a similar solid tumor in a different subject receiving a different treatment or receiving no treatment). The growth of a solid tumor in a subject can be assessed by a variety of different imaging methods, e.g., positron emission tomography, X-ray computed tomography, computed axial tomography, and magnetic resonance imaging.

Also provided herein are methods of decreasing the risk of developing a metastasis or developing an additional metastasis over a period of time in a subject identified as having a cancer (e.g., any of the exemplary cancers described herein) that include administering to the subject a therapeutically effective amount of any of the proteins described herein or any of the pharmaceutical compositions described herein (e.g., as compared to a subject having a similar cancer and receiving a different treatment or receiving no treatment). In some embodiments of any of the methods described herein, the metastasis or additional metastasis is one or more to a bone, lymph nodes, brain, lung, liver, skin, chest wall including bone, cartilage and soft tissue, abdominal cavity, contralateral breast, soft tissue, muscle, bone marrow, ovaries, adrenal glands, and pancreas.

In some embodiments of any of the methods described herein, the period of time is ~1 month to ~3 years (e.g., ~1-30, 1-24, 2-18, 1-12, 1-10, 1-8, 1-6, 1-5, 1-4, 1-3, 1-2, 2-36, 2-30, 2-24, 2-18, 2-12, 2-10, 2-8, 2-6, 2-5, 2-4, 2-3, 3-36, 3-30, 3-24, 3-18, 3-12, 3-10, 3-8, 3-6, 3-5, 3-4, 4-36, 4-30, 4-24, 4-18, 4-12, 4-10, 4-8, 4-6, 4-5, 5-36, 5-30, 5-24, 5-18, 5-12, 5-10, 5-8, 5-6, 6-36, 6-30, 6-24, 6-18, 6-12, 6-10, 6-8, 8-36, 8-30, 8-24, 8-18, 8-12, 8-10, 10-36, 10-30, 10-24, 10-18, 10-12, 12-36, 12-30, 12-24, 12-18, 18-36, 18-30, 18-24, 24-36, 24-30, or ~30-36 months).

In some embodiments, the risk of developing a metastasis or developing an additional metastasis over a period of time in a subject identified as having a cancer is decreased by ~1% to ~99% (e.g., or any of the subranges of this range described herein), e.g., as compared to the risk in a subject having a similar cancer receiving a different treatment or receiving no treatment.

Non-limiting examples of cancer include: acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms' tumor. Additional examples of cancer are known in the art.

In some embodiments, the patient is further administered one or more additional therapeutic agents (e.g., one or more of a chemotherapeutic agent, a recombinant cytokine or interleukin protein, a kinase inhibitor, and a checkpoint inhibitor). In some embodiments, the one or more additional therapeutic agents is administered to the patient at approximately the same time as any of the ABPCs described herein are administered to the patient. In some embodiments, the one or more additional therapeutic agents are administered to the patient after the administration of any of the ABPCs described herein to the patient. In some embodiments, the one or more additional therapeutic agents are administered to the patient before the administration of any of the ABPCs described herein to the patient.

In some embodiments of any of the methods described herein, the cancer is a solid cancer (e.g., breast cancer, prostate cancer, or non-small cell lung cancer).

### Compositions

Also provided herein are compositions (e.g., pharmaceutical compositions) that include at least one of any of the ABPCs described herein. In some embodiments, the compositions (e.g., pharmaceutical compositions) can be disposed in a sterile vial or a pre-loaded syringe.

In some embodiments, the compositions (e.g., pharmaceutical compositions) are formulated for different routes of administration (e.g., intravenous, subcutaneous, intramuscular, or intratumoral). In some embodiments, the compositions (e.g., pharmaceutical compositions) can include a pharmaceutically acceptable carrier (e.g., phosphate buffered saline). Single or multiple administrations of any of the pharmaceutical compositions described herein can be given to a subject depending on, for example: the dosage and frequency as required and tolerated by the patient. A dosage of the pharmaceutical composition should provide a sufficient quantity of the ABPC to effectively treat or ameliorate conditions, diseases, or symptoms. Also provided herein are methods of treating a subject having a cancer (e.g., any of the cancers described herein) that include administering a therapeutically effective amount of at least one of any of the compositions or pharmaceutical compositions provided herein.

### Kits

Also provided herein are kits that include any of the ABPCs described herein, any of the compositions described herein, or any of the pharmaceutical compositions described herein. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein.

### Protein Constructs

Also provided are protein constructs (PCs) that include: a first ABD that is capable of specifically binding PTK7 or an epitope of PTK7 presented on the surface of a target mammalian cell, where: (a) the dissociation rate of the first ABD at a pH of about 7.0 to about 8.0 (or any of the subranges of this range described herein) is faster than the dissociation rate at a pH of about 4.0 to about 6.5 (or any of the subranges of this range described herein); and/or (b) the dissociation constant (K_{D}) of the first ABD at a pH of about 7.0 to about 8.0 (or any of the subranges of this range) is greater than the K_{D} at a pH of about 4.0 to about 6.5.

Also provided herein are pharmaceutical compositions including any of the PCs described herein. Also provided herein are methods of treating a subject in need thereof that include administering a therapeutically effective amount of any of the PCs described herein to the subject.

### Methods of Improving pH Dependence of an Antigen-Binding Protein Construct

Also provided herein are methods of improving pH dependence of an antigen-binding protein construct, the method comprises providing a starting antigen-binding protein construct comprising an ABD and introducing one or more histidine amino acid substitutions into one or more CDRs of the ABD in the starting antigen-binding protein construct, wherein the method results in the generation of an antigen-binding protein construct having one or both of (a) an increased (e.g., at least a 0.1-fold increase to about a 100-fold increase, or any of the subranges of this range described herein) ratio of the dissociation rate of the ABD at a pH of about 4.0 to about 6.5 to the dissociation rate at a pH of about 7.0 to about 8.0, as compared to the starting antigen-binding protein construct, and (b) an increased (e.g., at least a 0.1-fold increase to about a 100-fold increase, or any of the subranges of this range described herein) ratio the dissociation constant (K_{D}) of the ABD at a pH of about 4.0 to about 6.5 to the K_{D} at a pH of about 7.0 to about 8.0, as compared to the starting antigen-binding protein construct.

The invention is further described in the following examples, which do not limit the scope of the invention described in the claims.

### EXAMPLES

### Example 1 Generation of PTK7 Binders and Engineering of pH Binding Dependence

pH-engineered A BPCs specific for PTK7 are generated using two methods. In the first approach, published monoclonal antibodies against PTK7 are used as a starting template for introduction of additional mutations that allow engineering of pH-dependent binding to PTK7 and i) enhanced endolysosomal accumulation of a conjugated toxin, as well as ii) enhanced PTK7 recycling to the cell surface. The second approach involves discovery of de novo ABPCs specific for PTK7 via antibody display methods from naive libraries or libraries with defined CDR compositions and screening under conditions designed for selection of pH-engineered ABPCs specific for PTK7. In either case, histidine residues play an important role in engineering pH-dependent binding proteins.

Histidine residues are at least partially protonated at a pH below 6.5 owing to its pKa of 6.0. Therefore, if a histidine side chain in an ABD participates in an electrostatic binding interaction with its antigen it will start to turn positively charged at a pH at or below 6.5. This could either weaken or enhance the binding affinity of the interaction at a pH below 6.5, based on the corresponding charge of and interactions with the antigen epitope. Thus, systematic introduction of histidines into antibody complementarity determining regions (CDRs) in an antibody or other binder library (e.g., an scFv library) can be used to identify substitutions that will affect an ABD's interaction with an antigen at lower pH values. The first approach therefore involves histidine-scanning of variable region sequences of published monoclonal antibodies to identify pH-dependent variants.

Multiple PTK7-binding monoclonal antibodies have been described in the literature and can be used as a template for engineering pH-dependent binding. Briefly, for a subset of the antibody sequences, CDRs in each chain are identified using the methods described by Kabat et al (Kabat et al. (1992) Sequences of Proteins of Immunological Interest (DIANE publishing) and IMGT (Lefranc MP (1999) “The IMGT unique numbering for Immunoglobulins, T cell receptors and Ig-like domains” The Immunologist 7, 132-136), and for each CDR, residues failing under either or both Kabat and IMGT CDR definitions were called as CDR residues. To engineer pH-dependent sequence variants, individual amino acid residues within the heavy chain and/or light chain CDRs are systematically substituted with a histidine, one at a time. In cases where the starting CDR residue is a histidine, it is mutated to an alanine. Antibody variants with only one histidine or alanine mutation in a heavy/light chain CDR are generated by co-transfection of Expi293 cells with a) one heavy chain or light chain sequence variant, and b) the corresponding starting ABPC (e.g., the starting PTK7-binding monoclonal antibody) light chain or heavy chain, respectively, using methods known to the art. After allowing for a period of protein expression, cell culture supernatants are collected, quantified, and the pH dependence of the variant is evaluated using biolayer interferometry (BLI) or other methods known to the art. Briefly, cell culture supernatants are normalized to an antibody expression level of 50 μg/mL, and captured on an anti-human Fe sensor (Forte Bio). A baseline is established using 1× kinetics buffer (Forte Bio), and the sensor is associated with 100 nM of PTK7 in IX PBS at pH 7.4 for 300 sec to generate an association curve. In the dissociation phase, the antibody-antigen complex on the sensor is exposed to IX PBS at either pH 5.5 or pH 7.4 for 300-500 sec. Association and dissociation phase curves are examined for the starting ABPC antibody and each corresponding antibody variant at pH 5.5 and pH 7.4 to inform on two criteria: a) enhanced dissociation (i.e., higher koff values) at pH 5.5 due to histidine or alanine substitution compared to the starting ABPC, and b) reduced dissociation at pH 7.4 (i.e., lower koff values) compared to pH 5.5 in the antibody variant itself and with the starting ABPC. Variants that show either enhanced dissociation at pH 5.5 or reduced dissociation at pH 7.4 or both are selected for further analysis. It is also noted that while some histidine and alanine mutations obliterate PTK7 binding, others are tolerated with little (e.g., less than 1-fold change in KD or dissociation rate) or no change in PTK7 binding kinetics, Especially because histidine is a large, positively charged amino acid, these histidine variants and alanine variants with no change are noted as positions that may tolerate a wide range of mutations and lead to antibodies with different sequence but similar binding properties, a designation that is not otherwise apparent. The variants selected for further analysis are expressed at a larger scale and purified using protein A affinity chromatography. Binding kinetics (kon and koff) of the purified starting ABPC and variant antibodies are measured at pH 5.5 and p-f 74 using Biacore (GE Healthcare). The ratio of the antibody's rate of dissociation (koff at pH 7.4 divided by koff at pH 5.5) is also used as a quantitative assessment of pH-dependent binding; similarly, the dissociation constant KD is calculated at both pH 5.5 and pH 7.4 as koff divided by kon and the ratio of the antibody's dissociation constant (KD at pH 7.4 divided by KD at pH 5.5) is also used as a quantitative assessment of pH-dependent binding. Antibodies with a rate of dissociation ratio less than that of the starting ABPC and/or a dissociation constant ratio less than that of the starting ABPC are selected for further assessment of combinatorial substitutions. Favorable histidine and/or alanine amino acid positions can also be combined to enhance pH dependence; this can be done by, e.g., combinatorially or rationally combining histidine and/or alanine substitutions on a given heavy or light chain that individually improve pH dependence, by, e.g., combinatorially or rationally combining modified heavy and light chains such that histidine and/or alanine substitutions are present on both chains, or combinations thereof. Such combinatorial variants are generated and tested/analyzed for differential pH dependence using the methods and protocols described herein, or others known to the art. Antibody variants that have the lowest rate of dissociation ratios and/or dissociation constant ratios are selected as candidates for further analysis (hereafter referred to as “pH-engineered ABPCs specific for PTK7”).

The second method for selection of pH-engineered ABPCs specific for PTK7 involves either screening libraries to identify de novo pH-dependent ABPCs specific for PTK7 or ABPCs that could serve as templates for engineering pH-dependent binding as described herein. Two types of libraries can be used for these selections: naive phage/yeast display antibody libraries (e.g., Fab, scFv, VHH, VL, or others known to the art) or phage/yeast display libraries where CDRs have been mutated to express a subset of amino acid residues. Libraries are screened against soluble recombinant PTK7 extracellular domains using methods known to the art with positive selection for variants that bind weakly (e.g., are eluted from beads) at pH 5.0 and bind strongly (e.g., are bound to beads) at pH 7.4. Three rounds of selections are performed. The final round of binders are screened using ELISA for binding to human PTK7 and cyno PTK7 and mouse PTK7 or via mean fluorescence intensity (MFI) in flow cytometric analysis. If more binders with cyno or murine cross-reactivity are desired, the final selection round can instead be performed on cyno PTK7 or murine PTK7. Selected binding proteins are subcloned into mammalian expression vectors and expressed as either full IgG proteins or Fe fusions in Expi293 cells. BLI analysis is performed as described herein for selection of pH-dependent binder variants and confirmed using Biacore.

### Example 2. In Vitro Demonstration of pH-Dependent Binding to PTK7, pH-Dependent Release of PTK7, Enhanced Endolysosomal Delivery in PTK7+ Cells, and Increased PTK7 Antigen Density in PTK7+ Cells after Exposure to pH-Engineered ABPCs Specific for PTK7 as Compared to Control ABPCs Specific for PTK7

As discussed herein, pH-engineered ABPCs specific for PTK7 exhibit the desirable property of decreased PTK7 binding at acidic pH (e.g., pH 5.0, pH 5.5), but enhanced binding at higher pH (e.g., pH 7.4), which enhances their accumulation in endolysosomes under physiological conditions.

pH-Dependent Binding to PTK7 on Cells

To demonstrate that pH-engineered ABPCs specific for PTK7 binds cell surface PTK7 at neutral pH, a cell surface binding assay is performed. A panel of human cells that are PTK7+ is assembled. Methods of identifying and quantifying gene expression (e.g., PTK7) for a given cell line are known to the art, and include, e.g., consulting the Cancer Cell Line Encyclopedia to ascertain the expression level and/or mutation status of a given gene in a tumor cell line), rtPCR, microarray, or RNA-Seq analysis, or cell staining with anti-PTK7 antibodies known in the art (e.g. cofetuzumab, 7C8, and 12C6). Cells are seeded at approximately 5-10,000 per well in 150 μL of pH 74 culture medium and incubated at 37° C. for 5 minutes at several doses (e.g., a two-fold dilution series) from 1 pM to 1 μM with one of the following antibodies: a known, control ABPC specific for PTK7 (e.g. cofetuzumab, 7C8, and 12C6), the pH-engineered ABPC specific for PTK7, and an appropriate negative isotype control mAb (e.g., Biolegend Purified Human IgG1 Isotype Control Recombinant Antibody, Cat #403501). Prior to the onset of the experiment, the binding properties of all antibodies are validated using methods known to the art. Following the 5 minute incubation, cells are fixed with 4% formaldehyde (20 min at room temperature) and incubated with an appropriate fluorophore-labeled secondary antibody (e.g., ThermoFisher Mouse anti-Human IgG1 Fc Secondary Antibody, Alexa Fluor 488, Cat #A-10631) for 60 minutes. Unbound reagents are washed with a series of PBS washes, and the cell panels are imaged using confocal microscopy. Upon analysis of the images, significant fluorescence can be observed on the surface of cells bound with the known, control ABPC specific for PTK7, as well as the pH-engineered ABPC specific for PTK7, but little surface binding can be observed for the isotype negative control. To isolate the effect of pH on surface binding, the same experiment is repeated twice, with the primary antibody incubation taking place at sequentially lower pH (e.g., pH 6.5 and 5.5 and 5.0). Analysis of the resulting confocal microscopy images can show significant fluorescence on the surface of cells bound with all mAbs tested, excepting the isotype negative control, and that this fluorescence decreases for the pH-engineered ABPC specific for PTK7 as the pH decreases. Alternatively, cells are analyzed for mean fluorescent intensity by flow cytometry using methods known in the art. A dissociation constant KD on cells at neutral pH of the antibodies analyzed is determined by nonlinear regression methods known in the art (e.g., a Scatchard plot). Taken together, the results can show that the pH engineering process results in the creation of a pH-engineered ABPC specific for PTK7 that is pH-dependent in its binding properties and that it more effectively binds at neutral pH as compared to more acidic pH. Other methods of assessing the pH dependence of the pH-engineered ABPCs specific for PTK7 are known in the art and include, e.g., using flow cytometry to measure ABPC surface binding.

pH-Dependent Release of PTK7 on Cells

To demonstrate that pH-engineered ABPCs specific for PTK7 are capable of releasing PTK7 at low pH after binding at a neutral pH, a variant of the cell surface binding assay described above is performed using methods known to the art (e.g., as generally described in Gera N. (2012) PLoS ONE 7(11): e48928). Briefly, an appropriate PTK7+ cell line is harvested and 50,000 cells per well are plated in a U-Bottomed 96-well microplate. Three conditions are tested-binding and secondary staining at pH 7.4, binding and secondary staining at pH 5.0, and binding at pH 7.4 followed by release at pH 5.0 for 30 minutes and secondary staining at pH 7.4. Both pH-engineered ABPCs specific for PTK7 as well as a control ABPC specific for PTK7 are tested. The cells are washed two times with 200 μL of FACS buffer (Ix PBS containing 3% Fetal Bovine Serum) at either pH 7.4 or 5.0 depending on the condition being tested. The purified protein samples are diluted into FACS buffer of the appropriate pH and added to the cells and allowed to bind for one hour on ice. After incubation with the primary antibodies the pH 7.4 and pH 5.0 conditions are washed twice as before, and then 100 μl of secondary rat anti-human Fc AF488 (BioLegend 410706) or other appropriate antibody, diluted 1:50 or anti My-Tag mouse mAb-AF488 (Cell Signaling Technologies 2279S) diluted 1:50 is added in FACS buffer of the appropriate pH, and incubated for 30 minutes on ice. The pH 5.0 release condition is washed twice with FACS buffer pH 74 and then resuspended in 100 μl of FACS buffer pH 5.0 and incubated on ice for 30 minutes, followed by secondary staining in FACS buffer pH 7.4 as described for the other conditions. The plates are washed twice as before and resuspended in 1% paraformaldehyde in the appropriate FACS buffer to fix them for flow cytometry analysis. All conditions are read on a flow cytometer (Accuri C6, BD Biosciences). Binding is observed as a shift in the FLI signal (as a MEI) versus secondary alone. Upon analysis of the data, it can be determined that both the pH-engineered ABPC specific for PTK7 as well as the control ABPC specific for PTK7 effectively bind the surface of PTK7+ cells at neutral pH, but the p-f-engineered ABPC specific for PTK7 binds poorly at pH 5.0; similarly, it can be determined that the pH-engineered ABPC specific for PTK7 binds effectively at pH 7.4, but then releases/unbinds PTK7 at pH 5.0.

Enhanced Endolysosomal Delivery in PTK7+ Cells of pH-Engineered ABPCs Specific for PTK7 as Compared to Control ABPCs Specific for PTK7 (pHrodo)

To verify and demonstrate that ABPCs specific for PTK7 achieve endolysosomal localization following cellular uptake, an internalization assay is performed using methods known to the art (e.g., Mahmutefendic et al., Int. J. Biochem. Cell Bio., 2011). Briefly, as described herein, a panel of human cells that express PTK7 highly is assembled using methods known to the art. Cells are plated, washed three times with PBS, and incubated at 37° C. for 60 minutes in media at neutral pH, with added concentrations of 2 micrograms per milliliter of a known, control ABPC specific for PTK7 (e.g., as described herein), the pH-engineered ABPC specific for PTK7, and an appropriate negative isotype control mAb (e.g., as described herein). In a subset of cells, validation of antibody internalization and endosomal localization is performed using methods known to the art; e.g., cells are fixed in 4% formaldehyde as described herein, permeabilized using TWEEN 20 or other methods known to the art (Jamur M C et al (2010) Permeabilization of cell membranes, Methods Mol Biol. 588:63-6), additionally stained with an endosomal marker, e.g., a fluorescent RAB11 antibody (RAB11 Antibody, Alexa Fluor 488, 3H18L5, ABfinity™ Rabbit Monoclonal), stained with an appropriate fluorescently labeled anti-human secondary antibody (e.g. as described herein), and imaged using confocal fluorescence microscopy, as described herein. Analysis of the confocal images can be used to show that both the pH-engineered ABPC specific for PTK7 as well as the control ABPC specific for PTK7 are internalized and accumulate in the endolysosomes.

To demonstrate that pH-engineered ABPCs specific for PTK7 achieve enhanced endolysosomal accumulation relative to a control ABPC specific for PTK7, a pHrodo-based internalization assay is performed using both a known, control ABPC specific for PTK7 as well as the pH-engineered ABPC specific for PTK7. The assay makes use of pHrodo™ iFL (P36014, ThermoFisher), a dye whose fluorescence increases with decreasing pH, such that its level of fluorescence outside the cell at neutral pH is lower than its level of fluorescence inside the acidic pH environment of endolysosomes. Briefly, an appropriate PTK7+ cell line is suspended in its recommended media and plated in a 24-well plate at a density of 2,000,000 cells/mL, 1 mL per well. While keeping the cells on ice, 1 mL of 2× pHrodo iFL-labeled antibody is added to each well, the well is pipetted/mixed five times, and the plate is incubated in a light-protected environment for 45 minutes, on ice. An identical but separate plate is also incubated on ice that is meant as a no-internalization negative control. Following this incubation, the experimental plate is moved to a 37° C. incubator, the negative control plate is kept on ice to slow or block internalization, and samples are taken at designated time points to create an internalization time course. Samples are placed into a U-bottom 96-well plate, and internalization is quenched via addition of 200 μL/well of ice-cold FACS buffer. The plates are spun down at 2000×g for 2 minutes, resuspended in 200 μL ice-cold FACS buffer, spun down again, and resuspended in FACS buffer a second time. Finally, the samples are loaded into a flow cytometer for read-out of cellular pHrodo fluorescence using excitation and emission wavelengths consistent with the excitation and emission maxima of the pHrodo iFL Red dye (566 nm and 590 nm, respectively). Upon completion of the flow cytometry experiment and analysis of the data, it can be observed that cells treated with the pH-engineered ABPC specific for PTK7 have a higher pHrodo iFL signal relative to a known, control ABPC specific for PTK7, indicating that pH-engineered ABPCs specific for PTK7 achieve enhanced endolysosomal accumulation relative to a control ABPC specific for PTK7.

Alternatively, to demonstrate that pH-engineered ABPCs specific for PTK7 achieve enhanced endolysosomal accumulation relative to a control ABPC specific for PTK7, a variation of the above-described experiment is performed. PTK7+ cells are plated, washed three times with PBS, and incubated at 37° C. for 60 minutes in media at neutral pH with added concentrations of 2 μg/mL of either pH-engineered ABPC specific for PTK7 or control ABPC specific for PTK7. Following incubation, cells are washed three times with PBS, fixed and permeabilized, and stained with a panel of appropriately selected antibodies that bind late endosomal markers as well as lysosomes (e.g., RAB7, and LAMP1; Cell Signaling Technology, Endosomal Marker Antibody Sampler Kit #12666; AbCam, Anti-LAMP2 antibody [GL2A7], ab13524). After primary antibody staining, cells are stained with an appropriate mixture of fluorescently labeled secondary antibodies (e.g., Goat Anti-Human IgG (H&L) Secondary Antibody (Alexa Fluor 647) Cat #A 21445, and Abcam Goat Anti-Rabbit IgG H&L (Alexa Fluor 488), Cat #ab150077), imaged using confocal fluorescence microscopy, and regions of co-localization of signal from PTK7-specific antibodies and endosomal markers are visualized and quantified. Upon analysis of the data, it can be revealed that there is increased co-localization of endolysosomal and PTK7-specific antibody signal in wells treated with the pH-engineered ABPCs specific for PTK7 as compared to wells treated with control ABPC specific for PTK7, and can thereby demonstrate that pH-engineered ABPCs specific for PTK7 achieve enhanced endolysosomal accumulation relative to control ABPC specific for PTK7.

Increased PTK7 Antigen Density in PTK7+ Cells after Exposure to pH-Engineered ABPCs Specific for PTK7 as Compared to Control ABPCs Specific for PTK7

To demonstrate that treatment of cells with the pH-engineered ABPCs specific for PTK7 does not result in a detectable reduction of the level of PTK7 on the surface of cells exposed to the pH-engineered ABPCs specific for PTK7, or that said treatment results in less of a reduction of the level of PTK7 on the surface of cells exposed to the pH-engineered ABPC specific for PTK7 versus a control ABPC specific for PTK7, an antigen density study is performed using flow cytometry. Briefly, 4.0×10≡cells that express PTK7 are plated per well in a 96-well plate in 100 μL media. Cells are treated with a titration from 1 pM to 1 μM of i) pH-engineered ABPCs specific for PTK7, ii) a first control ABPC specific for PTK7, iii) an appropriate isotype control, and iv) an untreated control. Cells are incubated for 2 hours at 37° C., at which point all cells are incubated with 200 nM of a fluorophore-labeled second control ABPC specific for PTK7 (e.g., as described herein) which has a different epitope (as determined by, e.g., competitive binding studies on cells) than either the first control ABPC specific for PTK7 or the pH-engineered ABPCs specific for PTK7 for 30 minutes at 4° C. Following this 30-minute incubation, the MIFI (MFI) of all cells is read out using, e.g., flow cytometry, using methods known to one of ordinary skill in the art. In parallel, a quantitative standard curve that can be used to quantify the presence of PTK7 on the surface of treated cells as a function of MFI is generated using a commercially available quantification kit (e.g., BD Biosciences PE Phycoerythrin Fluorescence Quantitation Kit, catalog #340495); the quantitative standard curve is created by following the manufacturer's instructions. Other methods of determining the absolute number of PTK7 on the cell surface are known in the art and include, e.g., use of radioisotopically labeled reagents. Upon analysis of the data, it can be revealed that at least one antibody concentration, cells treated with a control ABPC specific for PTK7 experience a reduction of the level of PTK7 on their surface, whereas cells treated with pH-engineered ABPCs specific for PTK7 experience a significantly smaller reduction or no reduction at all, both relative to the isotype and untreated controls.

### Example 3. Conjugation of pH-Engineered and Control ABPCs Specific for PTK7 to Cytotoxic Drugs

An antigen-binding protein construct conjugate (ADC) is made comprising the PTK7-binding IgG (hereafter, PTK7-IgG) described herein linked to monomethyl auristatin E (MMAE) via a valine-citrulline (vc) linker (hereafter, PTK7-IgG-DC). Conjugation of the antigen-binding protein construct with vcMMAE begins with a partial reduction of the PTK7-IgG followed by reaction with maleimidocaproyl-Val-Cit-PABC-MMAE (vcMMAE). The PTK7-IgG (20 mg/mL) is partially reduced by addition of TCEP (molar equivalents of TCEP:mAb is 2:1) followed by incubation at 0° C. overnight. The reduction reaction is then warmed to 20° C. To conjugate all of the thiols, vcMMAE is added to a final vcMMAE:reduced Cys molar ratio of 1:15. The conjugation reaction is carried out in the presence of 10% v/v of DMSO and allowed to proceed at 20° C. for 60 minutes.

After the conjugation reaction, excess free N(acetyl)-Cysteine (2 equivalents vs. vcMMAE charge) is added to quench unreacted vcMMAE to produce the Cys-Val-Cit-MMAE adduct. The Cys quenching reaction is allowed to proceed at 20° C. for approximately 30 minutes. The Cys-quenched reaction mixture is purified as per below. The above conjugation method can also be used to conjugate maleimidocaproyl monomethylauristatin F (mcMMAF) to an antigen-binding protein construct.

The PTK7-IgG-DC is purified using a batch purification method. The reaction mixture is treated with the appropriate amount of water washed Bu-HIC resin (ToyoPearl; Tosoh Biosciences), i.e., seven weights of resin is added to the mixture. The resin/reaction mixture is stirred for the appropriate time, and monitored by analytical hydrophobic interaction chromatography for removal of drug conjugate products, filtered through a coarse polypropylene filter, and washed by two bed volumes of a buffer (0.28 M sodium chloride, 7 mM potassium phosphate, pH 7). The combined filtrate and rinses are combined and analyzed for product profile by HIC HPLC. The combined filtrate and rinses are buffer exchanged by ultrafiltration/diafiltration (UF/DF) to 15 mM histidine, pH 6 with 10 diavolumes 15 nM histidine buffer.

A similar protocol can be used to conjugate DNA toxins such as SG3249 and SGD-1910 to PTK7-IgG (see Tiberghien A C et al (2016) Design and Synthesis of Tesirine, a Clinical Antibody-Drug Conjugate Pyrrolobenzodiazepine Dimer Payload, ACS Med Chem Lett 7:983-987). Briefly, for SG3249, PTK7-IgG (15 mg, 100 nanomoles) is diluted into 13.5 mL of a reduction buffer containing 10 mM sodium borate pH 8.4, 2.5 mM EDTA and a final antibody concentration of 1.11 mg/mL. A 10 mM solution of TCEP is added (1.5 molar equivalent/antibody, 150 nanomoles, 15 microliters) and the reduction mixture is heated at +37° C. for 1.5 hours in an incubator. After cooling down to room temperature, SG3249 is added as a DMSO solution (5 molar equivalent/antibody, 500 nanomoles, in 1.5 mL DMSO). The solution is mixed for 1.25 hours at room temperature, then the conjugation is quenched by addition of N-acetyl cysteine (1 micromole, 100 microliters at 10 mM), and injected into an AKTA™ Pure FPLC using a GE Healthcare HiLoad™ 26/600 column packed with Superdex 200 PG, and eluted with 2.6 mL/min of sterile-filtered phosphate-buffered saline (PBS). Fractions corresponding to the PTK7-IgG-DC monomer peak are pooled, concentrated using a 15 mL Amicon Ultracell 50 KDa MWCO spin filter, analyzed and sterile-filtered. UHPLC analysis on a Shimadzu Prominence system using a Phenomenex Aeris 3.6u XB-C18 150×2.1 mm column eluting with a gradient of water and acetonitrile on a reduced sample of PTK7-IgG-DC at 280 nm and 330 nm (SG3249 specific) can show a mixture of light and heavy chains attached to several molecules of SG3249, consistent with a drug-per-antibody ratio (DAR) of 1 to 4 molecules of SG3249 per antibody. UHPLC analysis on a Shimadzu Prominence system using a Phenomenex Yarra 3u SEC-3000 300 mm×4.60 mm column eluting with sterile-filtered SEC buffer containing 200 mM potassium phosphate pH 6.95, 250 mM potassium chloride and 10% isopropanol (v/v) on a sample of PTK7-IgG-DC at 280 nm can show a monomer purity of over 90% with no impurity detected. UHPLC SEC analysis allows determination of final PTK7-IgG-DC yield of greater than 30%.

Alternatively, methods to conjugate toxins to antibodies via lysine residues are known in the art (e.g., see Catcott K C et al (2016) Microscale screening of antibody libraries as maytansinoid antibody-drug conjugates, MAbs 8:513-23). In addition, similar methods to the above can be used to conjugate drugs and toxins to non-IgG formats with disulfide bonds, such as Vh-Fcs.

### Example 4. Demonstration of Enhanced Cytotoxicity of pH-Engineered ABPC ADCs Specific for PTK7 in PTK7+ Cells as Compared to a Control ABPC ADC Specific for PTK7

The cytotoxic activity of both pH-engineered ADCs specific for PTK7 (e.g., a pH-engineered PTK7-IgG-DC) and control ABPC ADCs specific for PTK7 (e.g., a control ABPC PTK7-IgG-DC) are separately evaluated on a panel of PTK7+ cell lines expressing a variety of antigen densities (e.g., as described herein) and a PTK7− cell line, selected using the methods described herein, and, optionally, cells expressing transgenic PTK7, e.g., HEK293 cells transfected with PTK7 using methods known in the art (e.g., Expi293™ Expression System Kit ThermoFisher Catalog number: A14635). For purposes of validation, prior to use, all cell lines are tested for expression of PTK7 using methods known to the art, e.g., qPCR, flow cytometry, mRNA RPKM, and antibody staining using anti-PTK7 antibodies known to the art (e.g., as described herein) followed by visualization of the stain using fluorescence microscopy, immunohistochemistry, flow cytometry, ELISA, or other methods known to the art. To evaluate the cytotoxicity of compounds, cells are seeded at approximately 10-40,000 per well in 150 microliters of culture medium, then treated with graded doses of compounds from 1 pM to 1 μM in quadruplicates at the initiation of the assay. Cytotoxicity assays are carried out for 96 hours after addition of test compounds. Fifty microliters of resazurin dye are added to each well during the last 4 to 6 hours of the incubation to assess viable cells at the end of culture. Dye reduction is determined by fluorescence spectrometry using the excitation and emission wavelengths of 535 nm and 590 nm, respectively. For analysis, the extent of resazurin reduction by the treated cells is compared to that of untreated control cells, and percent cytotoxicity is determined. Alternatively, a WST-8 kit is used to measure cytotoxicity per the manufacturer's instructions (e.g., Dojindo Molecular Technologies Catalog #CCK-8). IC50, the concentration at which half-maximal killing is observed, is calculated using curve-fitting methods known in the art. Upon analysis of the data, it can be determined that pH-engineered and control ABPC ADCs specific for PTK7 are substantially cytotoxic to one or more PTK7+ cell line, but less toxic to PTK7-cells. It also can be determined that pH-engineered ADCs specific for PTK7 are more cytotoxic to one or more PTK7+ cell lines than control ABPC ADCs specific for PTK7 because: a) they show greater depth of killing at one or more concentrations or, b) they show lower IC50 or, c) they show a greater ratio of their dissociation constant KD on cells at neutral pH (as described herein) divided by their IC50 on those same cells.

Additionally, the cytotoxic activity of ABPCs specific for PTK7 can be measured in a secondary ADC assay. Secondary ADC assays are known in the art (e.g., Moradec Cat #αHFc-NC-MMAF and Cat #αHFc-CL-MMAE, and associated manufacturer's instructions). Briefly, the assay is carried out as in the previous paragraph, except the ABPC specific for PTK7 is substituted for the ADC specific for PTK7, and to evaluate the cytotoxicity of compounds, cells are seeded at approximately 10-40,000 per well in 150 microliters of culture medium, then treated with graded doses of ABPC specific for PTK7 from 1 pM to 1 μM (final concentration in culture medium, having been pre-mixed with 100 nM, final concentration in culture medium, of Moradec Cat #αHFc-NC-MMAF secondary ADC reagent and pre-incubated at 37° C. for 30 min before addition of the mixture to the culture medium) in quadruplicates at the initiation of the assay.

The cytotoxic activity of pH-engineered ADCs specific for PTK7 and control ABPC ADCs specific for PTK7 conjugates, as well as ABPCs specific for PTK7 in a secondary ADC assay, are additionally measured by a cell proliferation assay employing the following protocol (Promega Corp. Technical Bulletin TB288; Mendoza et al., Cancer Res. 62:5485-5488, 2002):
1. An aliquot of 100 microliters of cell culture containing about 104 cells (e.g., PTK7+ cells as described herein) in medium is deposited in each well of a 96-well, opaque-walled plate.
2. Control wells are prepared containing medium and without cells.
3. ADC specific for PTK7 is added to the experimental wells at a range of concentrations from 1 pM-1 uM and incubated for 1-5 days. Alternatively, in a secondary ADC assay, 100 nM secondary ADC reagent (final concentration in culture medium, Moradec Cat #αHFc-NC-MMAF) and ABPC specific for PTK7 at a range of concentrations from 1 pM-1 uM (final concentration in culture medium) are pre-mixed and pre-incubated at 37° C. for 30 min before addition of the mixture to the culture medium, and incubated for 1-5 days.
4. The plates are equilibrated to RT for approximately 30 minutes.
5. A volume of CellTiter-Glo Reagent equal to the volume of cell culture medium present in each well is added.
6. The contents are mixed for 2 minutes on an orbital shaker to induce cell lysis.
7. The plate is incubated at RT for 10 minutes to stabilize the luminescence signal.
8. Luminescence is recorded; reported in graphs as RLU=relative luminescence units.

### Example 5, Demonstration of Enhanced Toxin Liberation of pH-Engineered ABPC ADCs Specific for PTK7 in PTK7+ Cells as Compared to a Control ABPC ADC Specific for PTK7

The pH-engineered ADCs specific for PTK7 (e.g., a pH-engineered PTK7-IgG-DC) can also demonstrate increased toxin liberation in PTK7+ cells as compared to a control ABPC ADC specific for PTK7 (e.g., a control ABPC PTK7-IgG-DC). After treatment of PTK7+ cells with pH-engineered and control ABPC ADCs specific for PTK7 as described herein, an LC-MS/MS method is used to quantify unconjugated (i.e., liberated) MMAE in treated PTK7+ cells (Singh, A. P. and Shah, D. K. Drug Metabolism and Disposition 45.11 (2017): 1120-1132.) An LC-MS/MS system with electrospray interphase and triple quadrupole mass spectrometer is used. For the detection of MMAE, a XBridge BEH Amide column (Waters, Milford, MA) is used with mobile phase A as water (with 5 mM ammonium formate and 0.1% formic acid) and mobile phase B as 95:5 acetonitrile/water (with 0.1% formic acid and 1 mM ammonium formate), using a gradient at a flow rate of 0.25 mL/min at 40° C. The total duration of the chromatographic run is 12 minutes, where two MRN scans (718.5/686.5 and 718.5/152.1 amu) are monitored. Deuterated (d8) MMAE. (MICE MedChem Express, Monmouth Junction, NJ) is used as an internal standard. First, an equation for quantifying unconjugated MMAE in a biological sample is derived by dividing the peak area for each drug standard by the peak area obtained for the internal standard. The resultant peak area ratios are then plotted as a function of the standard concentrations, and data points are fitted to the curve using linear regression. Three QC samples are included in the low, middle, and upper ranges of the standard curve to assess the predictive capability of the developed standard curve. The standard curves obtained are then used to deduce the observed concentrations of MMAE in a biologic sample. For measurement of MMAE concentration, treated cell samples are pelleted and reconstituted in fresh media to a final concentration of 0.25 million cells/100 μL. Samples are spiked with d8-MMAE (1 ng/ml) before performing cell lysis by the addition of a 2-fold volume of ice-cold methanol followed by freeze-thaw cycle of 45 minutes at −20° C. The final cell lysate is obtained by centrifuging the samples at 13,000 rpm for 15 minutes at 4° C. followed by collection of supernatant. For the preparation of standards and QC samples, a fresh cell suspension (0.25 million/100 μl) is spiked with known concentrations of MMAE and internal standard (d8-MMAE) before a procedure similar to the cell lysis mentioned above. The resulting cell lysates are then evaporated and reconstituted in mobile phase B before injection into LC-MS/MS. The concentration of unconjugated MMAE in lysates of PTK7+ cells treated with pH-engineered ADCs specific for PTK7 is observed to be greater than that in PTK7+ cells treated with control ABPC ADC specific for PTK7.

For tubulin-inhibiting toxins, toxin liberation is also assessed by monitoring of cell viability and cell cycle phase. ~ 2.0×10\hat{ }5 PTK7+ cells are plated in a 96-well flat bottom plate and treated with pH-engineered and control ABPC ADCs specific for PTK7 as described herein. After treatment, cells are transferred to a 96-round bottom plate, and the plate is centrifuged at 400 rcf for 2 min to decant supernatant. Decanted cells are stained with Live/Dead eFluor 660. Cells are then centrifuged and washed with FACS buffer (PBS with 2% FBS), after which cell cycle distribution is analyzed with a BD Cycletest™ Plus DNA Kit (cat #340242). Briefly, cells are re-suspended in 76 μl Solution A and incubated for 10 min at room temperature. 61 μL Solution B is then added, and cells are incubated for another 10 min at room temperature. Finally, 61 μL of cold Solution C is added, and cells are again incubated for 10 min at room temp. Immediately after the last incubation step, cells are analyzed by flow cytometry (without washing) at a flow rate of 10 μL/sec. Increased G2/M-phase arrest can be observed with exposure to pH-engineered ADCs specific for PTK7 as compared to control ABPC ADC specific for PTK7.

For DNA-damaging toxins (e.g., pyrrolobenzodiazepine or “PBD”), DNA damage is assessed by measuring the phosphorylated histone H2AX (γH2AX). H2AX is normally phosphorylated in response to double-strand breaks in DNA; however, increased levels γH2AX may also be observed as a result of treatment with DNA-cross-linking toxins such as PBD or cisplatin (Huang, X. et al. 2004, Cytometry Part A 58A, 99-110). PTK7+ cells are treated with pH-engineered and control ABPC ADCs specific for PTK7 as described herein. After treatment, cells are rinsed with PBS, and then fixed in suspension in 1% methanol-free formaldehyde (Polysciences, Warrington, PA) in PBS at 0° C. for 15 min. Cells are resuspended in 70% ethanol for at least 2 h at −20° C. Cells are then washed twice in PBS and suspended in 0.2% Triton X-100 (Sigma) in a 1% (w/v) solution of BSA (Sigma) in PBS for 30 min to suppress nonspecific Ab binding. Cells are centrifuged again (200 g, 5 min) and the cell pellet is suspended in 100 μL of 1% BSA containing 1:800 diluted anti-histone γH2AX polyclonal Ab (Trevigen, Gaithersburg, MD). The cells are then incubated overnight at 4° C., washed twice with PBS, and resuspended in 100 μL of 1:30 diluted FITC-conjugated F(ab′) 2 fragment of swine anti-rabbit immunoglobulin (DAKO, Carpinteria, CA) for 30 min at room temperature in the dark. The cells are then counterstained with 5 μg/mL of PI (Molecular Probes, Eugene, OR) dissolved in PBS containing 100 μg/mL of DNase-free RNase A (Sigma), for 20 min at room temperature. Cellular fluorescence of the FITC γH2AX signal and the PI counterstain are measured using flow cytometry using methods known in the art. When comparing cells within the same stage of the cell cycle (based on total DNA content), treated PTK7+ cells can be observed to have an increased FITC γH2AX signal relative to untreated PTK7+ cells (which serve as a baseline). Furthermore, PTK7+ cells treated with pH-engineered ADCs specific for PTK7 can be observed to have a greater increase in levels of γH2AX over baseline than cells treated with a control ABPC ADC specific for PTK7. In addition to the γH2AX assay, DNA cross-linking can be more directly assessed with a Comet assay (Chandna, S. (2004) Cytometry 61A, 127-133).

In addition, as disclosed herein, pH-engineered and control ABPCs can be assayed using the methods in this example without direct conjugation by performing a secondary ADC assay instead of using primary conjugated ADCs.

### Example 6. Demonstration of Increased or Decreased Half-Life of pH-Engineered ABPCs Specific for PTK7 as Compared to a Control ABPC Specific for PTK7

One of the surprising aspects of the pH-engineered ABPCs specific for PTK7 described by the invention can be their ability to facilitate increased dissociation of ABPCs from the PTK7 within the endosome or lysosome resulting in a decreased or increased serum half-life relative to control ABPCs specific for PTK7 or ABPCs that are not specific for PTK7. To demonstrate these properties, a series of animal studies in mice and/or monkeys is performed using pH-engineered ABPC specific for PTK7 and control ABPC specific for PTK7 using methods known to the art (e.g., Gupta, P., et al. (2016), mAbs, 8:5, 991-997). Briefly, to conduct mouse studies, a single intravenous bolus (e.g., 5 mg/kg) of either pH-engineered ABPC specific for PTK7 or control ABPC specific for PTK7 is administered via tail vein to two groups of NOD SCID mice (e.g. Jackson Labs NOD.CB17-Prkdescid/J Stock No: 001303) xenografted with a PTK7+ cell line (e.g., as described herein). Xenografted mice are prepared by growing 1-5 million PTK7+ cells in vitro and inoculating subcutaneously into the right flank of the mouse. Tumors are size matched at 300 mm³. Measurements of the length (L) and width (W) of the tumors are taken via electronic caliper and the volume is calculated according to the following equation: V=L×W\hat{ }2/2. Blood samples are collected via retro-orbital bleeds from each group at each of the following time points: 15 m, 30 m, 1 h, 8 h, 24 h, and 3 d, 7 d, 10 d, 14 d, 17 d, 21 d, and 28 d. Samples are processed to collect serum, and antibody concentrations are quantified using ELISA or other methods known to the art (e.g., PAC assay or MAC assay; Fischer, S. K. et al. (2012), mAbs, 4:5, 623-631, utilizing, e.g., anti-human Fc antibody Jackson ImmunoResearch Labs, Cat #109-006-006). Antibody concentrations of pH-engineered ABPC specific for PTK7 and control ABPC specific for PTK7 are plotted as a function of time. Upon analysis of the data, it can be observed that the pH-engineered ABPC specific for PTK7 has a significantly longer or shorter serum half-life relative to control ABPC specific for PTK7. If the pH-engineered and control ABPCs specific for PTK7 are cross-reactive with the mouse homolog of PTK7, a similar experiment can be repeated with non-xenografted mice.

Optionally, if the pH-engineered and control ABPCs specific for PTK7 are cross-reactive with the cynomolgus monkey homolog of PTK7, a similar experiment can be performed on monkeys (e.g., cynomolgus monkeys). An equal number of male and female monkeys (e.g., n=1-2 each) are administered a bolus of either pH-engineered ABPC specific for PTK7 or control ABPC specific for PTK7 at a dose of, e.g., 1 mg/kg via saphenous vein injection. Alternatively, several different doses of PTK7-binding protein are administered across a group of several monkeys. Blood samples are collected via the peripheral vein or femoral vein at intervals similar to those described above, and analyzed for the presence of either pH-engineered ABPC specific for PTK7 or control ABPC specific for PTK7 using methods known to the art (e.g., ELISA). Upon analysis of the data, it can be observed that pH-engineered ABPC specific for PTK7 has a significantly longer or shorter serum half-life relative to control ABPC specific for PTK7. In some cases, this effect is observed only in certain doses, whereas in others it is observed across doses. In particular cases, increased half-life is desirable, especially where less frequent patient dosing is advantageous.

In addition, the half-life of pH-engineered and control ABPC ADCs specific for PTK7 can be assessed using the above methods by substituting pH-engineered and control ABPC ADCs specific for PTK7 for the pH-engineered and control ABPCs specific for PTK7 (i.e., studying the ABPCs after conjugation to a drug or toxin, as described herein).

### Example 7, Increased Potency of pH-Engineered ADCs Specific for PTK7 vs. a Control ABPC ADC Specific for PTK7 in Mouse Xenograft Models

The enhanced anti-tumor activity of the pH-engineered ADCs specific for PTK7 against PTK7+ tumors can be demonstrated in a subcutaneous xenograft model of PTK7+ cells. For the experiments, 1-5 million PTK7+ cells are grown in vitro and inoculated subcutaneously per mouse into the right flank of female immunodeficient (e.g., SCID-Beige or NOD scid) mice. Tumors are size matched at 100-200 mm³, and dosed intraperitoneally (IP) (1 dose given every ~4-7 days for a total of ~2-6 doses). Measurements of the length (L) and width (W) of the tumors are taken via electronic caliper and the volume is calculated according to the following equation: V=L×W\hat{ }2/2. A bolus (e.g., 5 mg/kg) of either pH-engineered ADC specific for PTK7 or control ABPC ADC specific for PTK7 is administered via tail vein. Tumor growth inhibition (TGI) and tumor growth delay (TGD) and survival are significantly improved with administration of pH-engineered ADC specific for PTK7 compared to administration of control ABPC ADC specific for PTK7 at the same regimen.

Optionally, spread of tumor cells into the various tissues is determined in sacrificed animals. Metastasis is measured according to Schneider, T., et al., Clin. Exp. Metas. 19 (2002) 571-582. Briefly, tissues are harvested and human Alu sequences are quantified by real-time PCR. Higher human DNA levels, quantified by real-time PCR, correspond to higher levels of metastasis. Levels of human Alu sequences (correlating to invasion of tumor cells into secondary tissue) are significantly lower in animals treated with pH-engineered ADC specific for PTK7, corresponding to reduced metastasis, compared to mice treated with control ABPC ADC specific for PTK7 at the same regimen. Alternatively, the enhanced anti-tumor activity of the pH-engineered ADC specific for PTK7 can be shown in PTK7+ patient-derived xenograft models.

### Example 8. Creation of a pH-Engineered Bispecific PTK7 Bispecific ABPC and Demonstration of Exemplary Properties as Compared to a Control Bispecific ABPC

To create a pH-engineered ABPC specific for PTK7 with modified toxicity and internalization properties, a bispecific antibody that binds two different epitopes on PTK7 is constructed. It is known in the art that biparatopic antibodies can show increased antigen-dependent internalization, and are therefore useful for applications such as antibody-drug conjugates (e.g., see Li et al (2016) A Biparatopic HER2-Targeting Antibody-Drug Conjugate Induces Tumor Regression in Primary Models Refractory to or Ineligible for HER2-Targeted Therapy, Cancer Cell 29:117-29). Briefly, a pH-engineered PTK7×PTK7 bispecific, biparatopic ABPC specific for PTK7 is assembled using light chain/heavy chain pairs from two different pH-engineered ABPCs specific for PTK7, each of which binds a distinct epitope on PTK7 that does not overlap with the other epitope. A set of pH-engineered ABPCs specific for PTK7 that bind non-overlapping epitopes are discovered, e.g., using the methods described herein, or others known to one of ordinary skill in the art. Briefly, two binders are selected on the basis that they bind substantially different epitopes on PTK7, as determined by, e.g., a binding competition assay as in Abdiche Y N et al (2009) Exploring blocking assays using Octet, ProteOn, and Biacore biosensors, Anal Biochem 386:172-80. Alternatively, briefly, as described herein, cell culture supernatants of cells transfected with a first ABPC specific for PTK7 are normalized to an antibody expression level of 50 μg/mL, and captured on an anti-human Fc sensor (Forte Bio). A baseline is established using 1× kinetics buffer (Forte Bio), and the sensor is associated with 50 nM of PTK7 in 1×PBS (that has been mixed and pre-incubated for 30 min at 37° C. with a second ABPC specific for PTK7 transfection supernatant or the first ABPC specific for PTK7 transfection supernatant, both normalized to 50 μg/mL) at pH 7.4 for 300 sec to generate an association curve. If the association rate in the presence of the second ABPC specific for PTK7 is significantly faster (as calculated by the instrument software, or as seen by an elevated level of association over time) than the association rate in the presence of the first ABPC specific for PTK7, then the second ABPC specific for PTK7 is deemed to bind a non-overlapping epitope of PTK7. Optionally, each antibody is screened for its internalization properties when bound to its epitope on a cell expressing PTK7, and well-internalizing antibodies are selected. Assays for determining the internalization rate of a molecule present on the surface of a cell are known to the art. See, e.g., Wiley et al. (1991) J. Biol. Chem. 266:11083-11094; and Sorkin and Duex (2010) Curr. Protoc. Cell Biol. Chapter, Unit-15.14; Vainshtein et al. (2015) Pharm Res. 32:286-299. Once selected, heavy and light chain constructs with engineered mutations for heavy and light chain pairing (Spiess et al., “Alternative molecular formats and therapeutic applications of bispecific antibodies,” 2015) are synthesized for both arms. Bispecific ABPCs specific for PTK7 are produced by co-expression of corresponding heavy and light chain plasmids in, e.g., Expi293 cells. Cell culture supernatants are harvested and subjected to Protein A purification. Heterodimeric ABPCs specific for PTK7 are separated from homodimeric species via additional purification steps such as ion exchange chromatography, hydrophobic interaction chromatography, and mixed mode chromatography. The purified pH-engineered PTK7×PTK7 bispecific, biparatopic ABPCs specific for PTK7 are characterized via mass spectrometry to confirm the purity and absence of homodimeric species and size exclusion chromatography to confirm the presence of monomeric antigen-binding protein construct species. For the product antibody, binding to the PTK7 is confirmed via Biacore analysis. Other methods of bispecific antibody production are known to the art and could also be used to create a bispecific antibody, e.g., the PTK7×PTK7 bispecific, biparatopic ABPCs specific for PTK7 described herein (e.g., Labrijn et al (2014) “Controlled Fab-arm exchange for the generation of stable bispecific IgG1” Nature Protocols 9:2450-2463, accessed at Nature's website, as would be apparent to one of ordinary skill in the art. Alternatively, instead of a PTK7×PTK7 ABPC specific for PTK7, a pH-engineered PTK7×BINDER ABPC specific for PTK7 can be constructed using similar methods apparent to one skilled in the art, where BINDER is any antibody that has been published in the art or discovered using methods like those herein or those known in the art (e.g., display-based or immunization-based methods).

Next, exemplary properties of pH-engineered PTK7×PTK7 ABPCs specific for PTK7 can be demonstrated using the methods described herein, with the appropriate control being a control ABPC monospecific or bispecific ABPC specific for PTK7. Briefly, it can be shown that, as compared to a control, the pH-engineered PTK7×PTK7 ABPCs specific for PTK7: a) bind in a pH-dependent manner to cells, e.g., bind at a neutral pH but not an acidic pH and b) release from cells in a pH-dependent manner, e.g. bind at a neutral pH and release at an acidic pH and c) show enhanced endolysosomal accumulation in PTK7+ cells and d) show increased PTK7 antigen density after exposure to PTK7+ cells and e) when conjugated to a toxin, show increased cytotoxicity to PTK7+ cells and f) when conjugated to a toxin, show increased toxin liberation when incubated with PTK7+ cells and g) show decreased half-life when exposed to PTK7 antigen in a relevant animal model and h) when conjugated to a toxin, show increased efficacy in a mouse xenograft model of PTK7+ cells. Similarly, the exemplary properties of pH-engineered PTK7×BINDER ABPCs specific for PTK7 can be demonstrated using the methods described herein, with the appropriate control being a control ABPC PTK7×BINDER bispecific ABPC specific for PTK7.

### Example 9. Construction and Screening of pH-Engineered PTK7 ABPCs

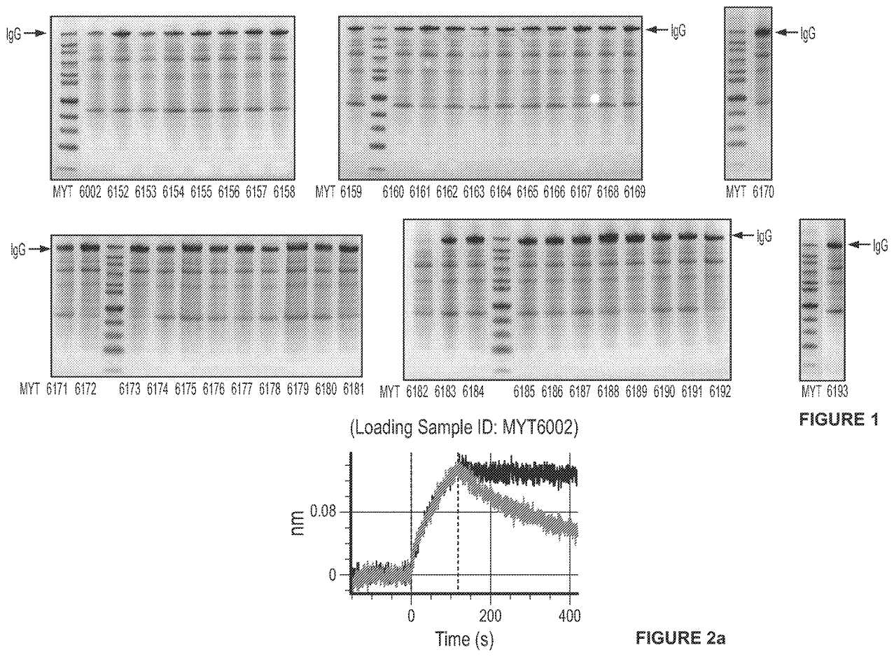

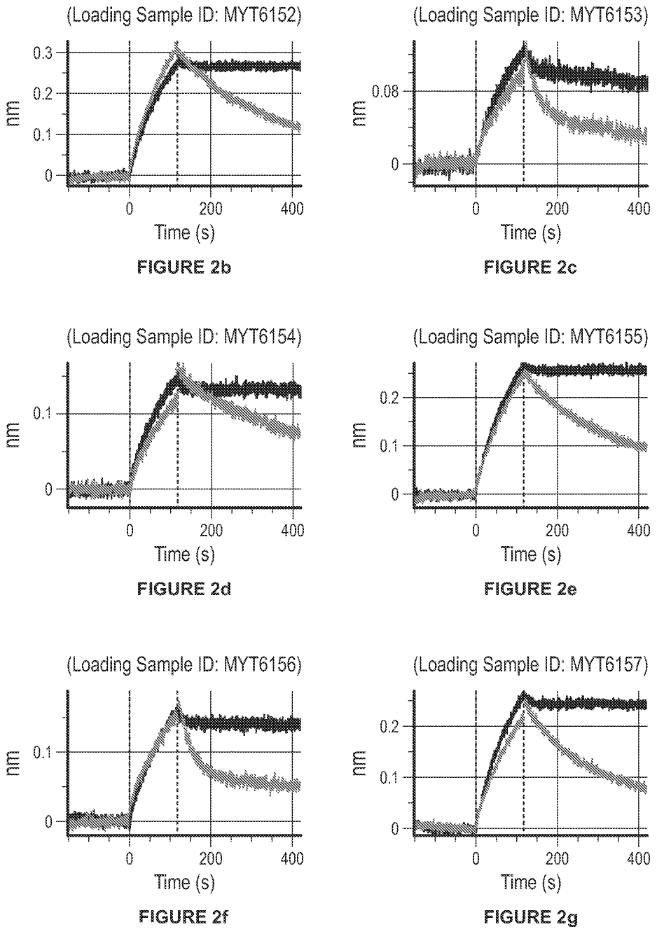

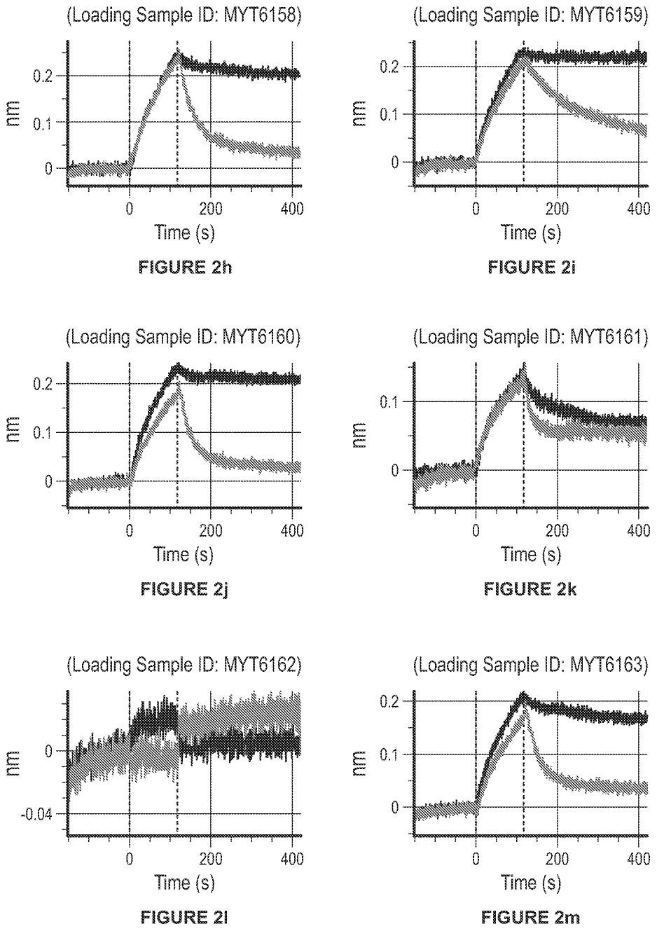

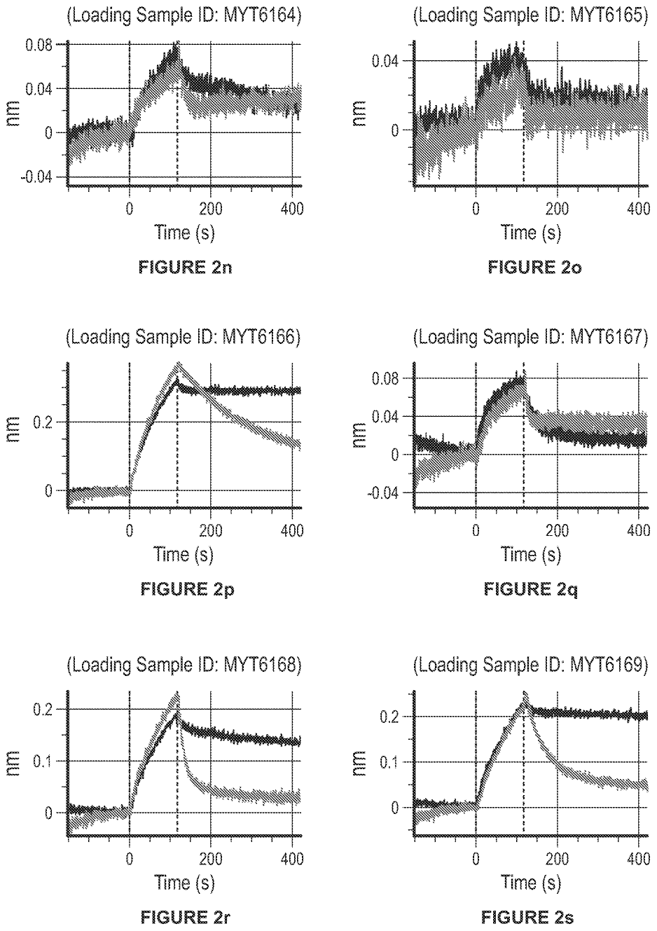

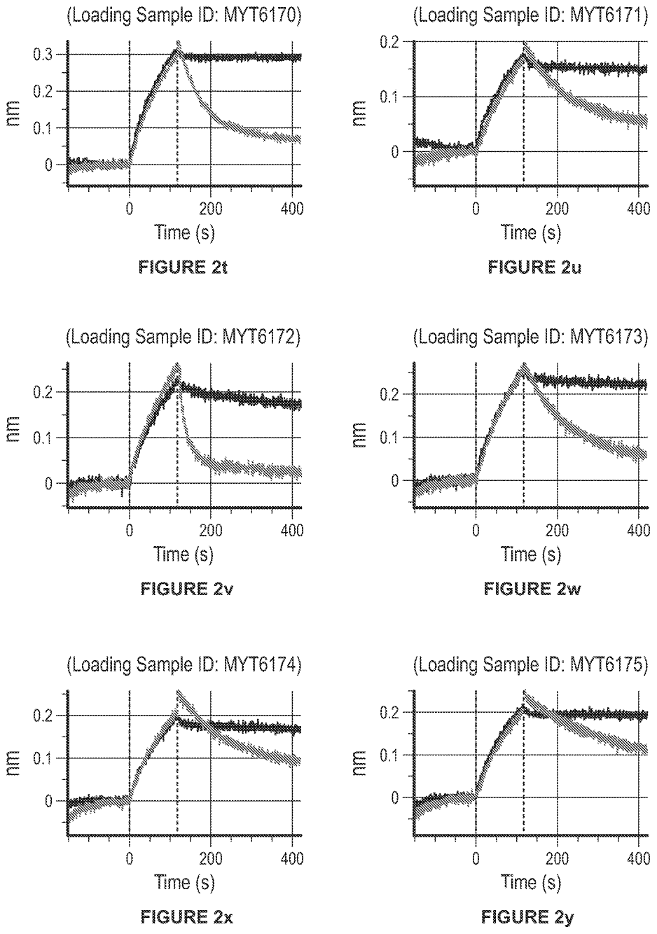

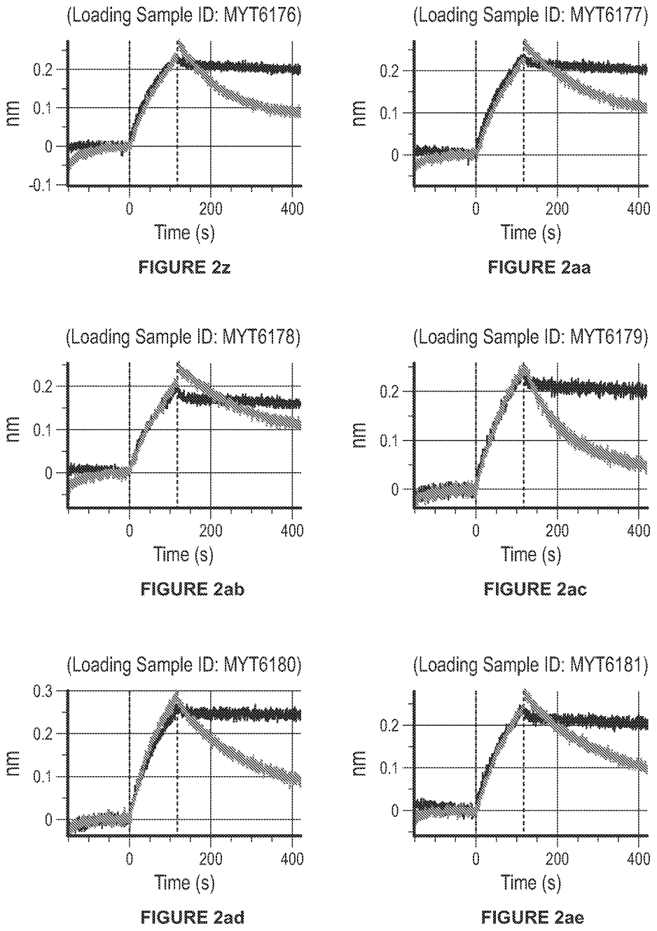

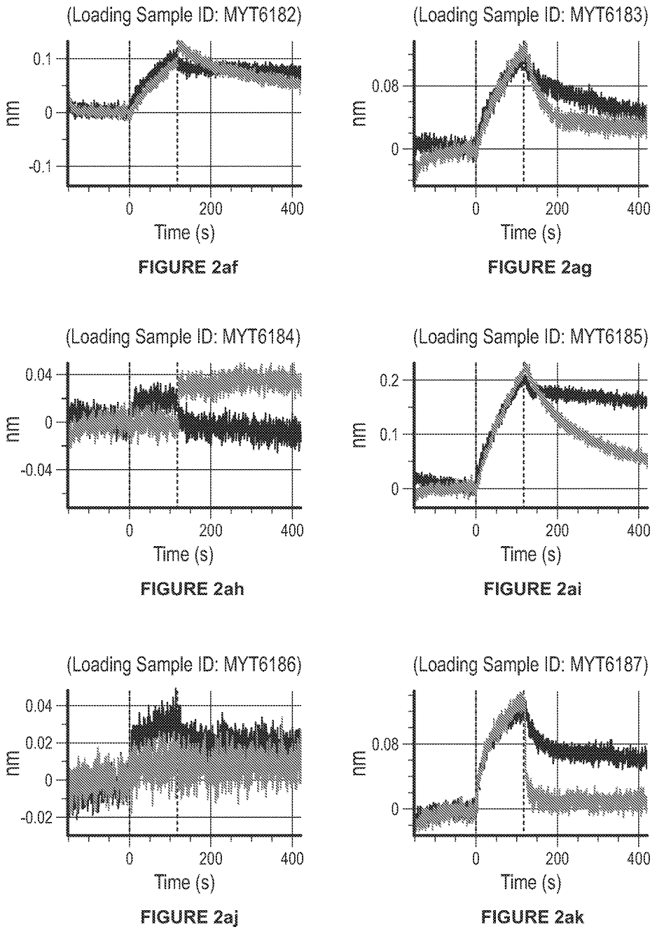

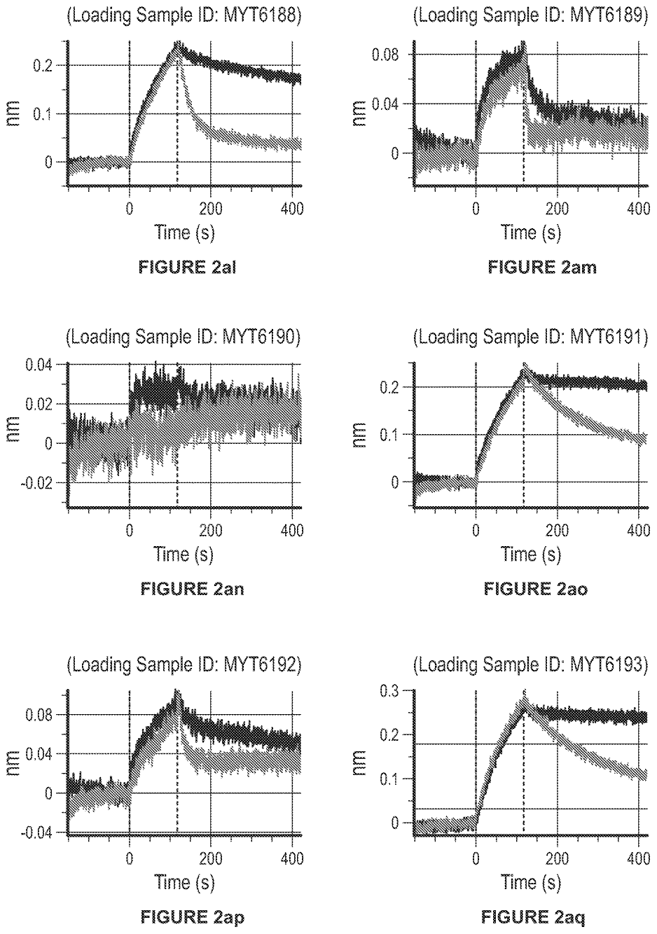

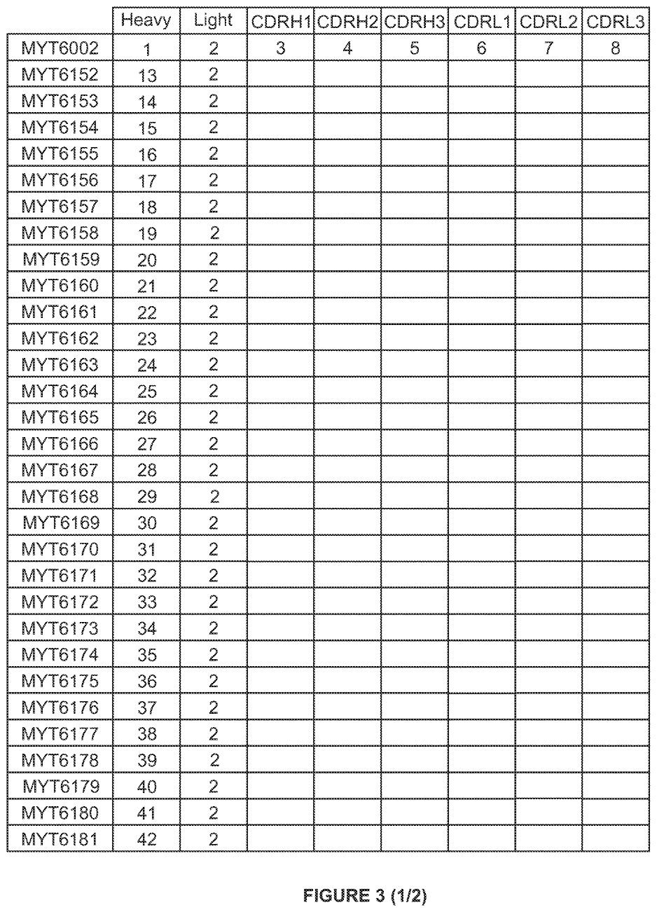

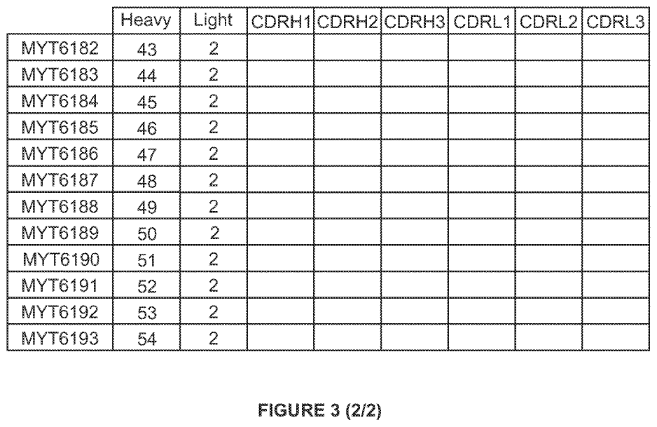

Multiple PTK7-binding monoclonal antibodies have been described in the literature and can be used as a template for engineering pH-dependent binding [Damelin et. al., A PTK7-Targeted Antibody-Drug Conjugate Reduces Tumor-Initiating Cells and Induces Sustained Tumor Regressions, Sci Transl Med. 2017 Jan. 11; 9 (372): eaag2611.]. We selected cofetuzumab (Heavy chain SEQ ID NO: 1, Light chain SEQ ID NO: 2) as a PTK7-binding monoclonal antibody for pH engineering via histidine scanning. Briefly, CDRs in the heavy chain were identified using the methods described by Kabat et al (Kabat et al. (1992) Sequences of Proteins of Immunological Interest, DIANE publishing) and IMGT (Lefranc MP (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the heavy chain CDRs were systematically substituted with a histidine, one at a time (MYT6152-MYT6193). In cases where the starting CDR residue was a histidine, it was mutated to an alanine. Antibody variants with only one histidine or alanine mutation in a heavy chain CDR were generated by co-transfection of Expi293 cells with a) one heavy chain sequence variant, and b) the corresponding starting ABPC light chain using methods known to the art. After allowing for four days of protein expression, cell culture supernatants were collected, quantified by SDS-PAGE analysis (FIG. 1), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. The resulting diluted supernatants were then captured on an anti-human Fc sensor (Forte Bio). A baseline was established using 1×PBST (50 mM Potassium Phosphate Buffer+150 mM NaCl+0.05% Tween 20) pH 7.4, and the sensor was associated with 50 nM of PTK7 (Human PTK7/CCK4 Protein, His Tag, Acro Biosystems PT7-H52H3, Lot No. 4313a-212BF1-VH) in 1×PBST pH 7.4 for 120 sec to generate an association curve. In the dissociation phase, the antibody-antigen complex on the sensor was exposed to 1×PBST pH 7.4 or pH 5.4, for 300 sec. Association and dissociation phase curves were examined for the starting ABPC antibody (with no substitutions) and each corresponding antibody variant at pH 5.4 and pH 7.4 to inform on two criteria: a) enhanced dissociation (i.e., higher koff values) at pH 5.4 due to histidine or alanine substitution compared to the starting ABPC, (with no substitutions), and b) reduced dissociation at pH 7.4 (i.e., lower koff values) compared to pH 5.4 in the antibody variant itself and with the starting ABPC (with no substitutions). Heavy chain variants that showed either enhanced dissociation at pH 5.4 or reduced dissociation at pH 7.4 or both (as compared to starting ABPC) as shown in FIG. 2 were selected for further analysis (e.g., MYT6153, MYT6156, MYT6157, MYT6158, MYT6159, MYT6160, MYT6161, MYT6163, MYT6164, MYT6168, MYT6169, MYT6170, MYT6172, MYT6173, MYT6179, MYT6183, MYT6185, MYT6187, MYT6188, and MYT6192). It was also noted that while some histidine and alanine mutations obliterated PTK7 binding (e.g., MYT6162, MYT6165, MYT6184, MYT6186, and MYT6190), others were tolerated with little (e.g., less than 1-fold change in dissociation constant KD or dissociation rate) or no change in PTK7 binding kinetics (e.g., MYT6152, MYT6154, MYT6155, MYT6166, MYT6171, MYT6174, MYT6175, MYT6176, MYT6177, MYT6178, MYT6180, MYT6181, MYT6182, MYT6191, and MYT6193).

Especially because histidine is a large, positively charged amino acid, these variants with no change were noted as positions in the heavy chain that may tolerate a wide range of mutations and lead to antibodies with different sequence but similar binding properties, a designation that is not otherwise apparent.

### Example 10. Construction and Screening of pH-Engineered PTK7 ABPCs

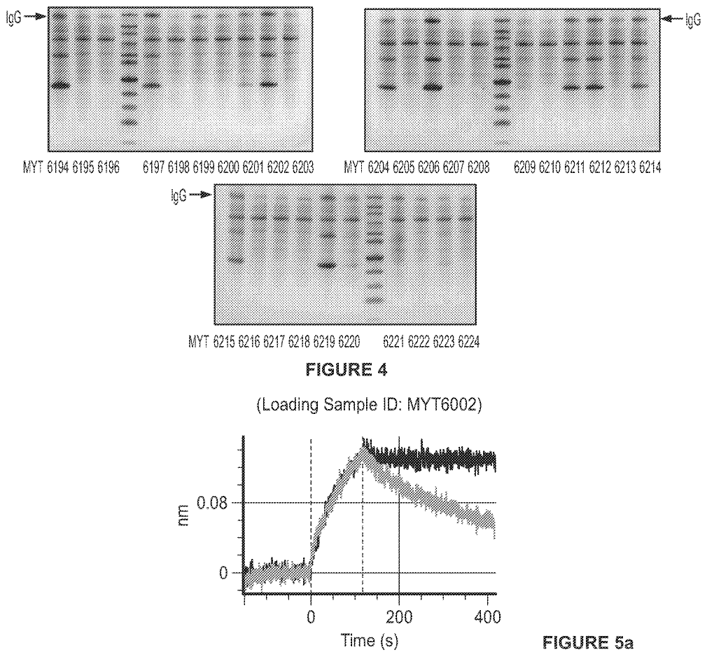

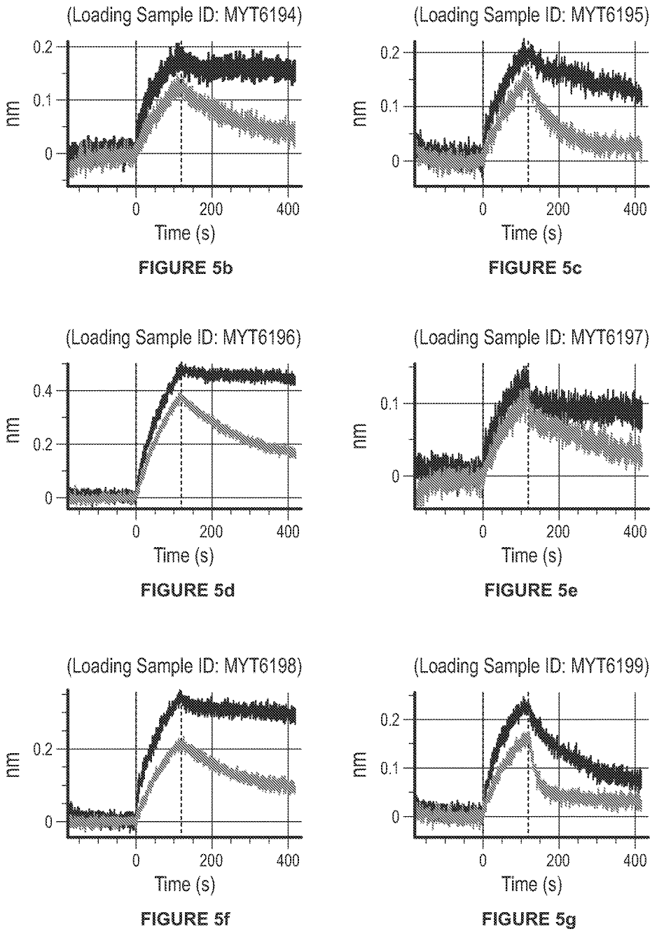

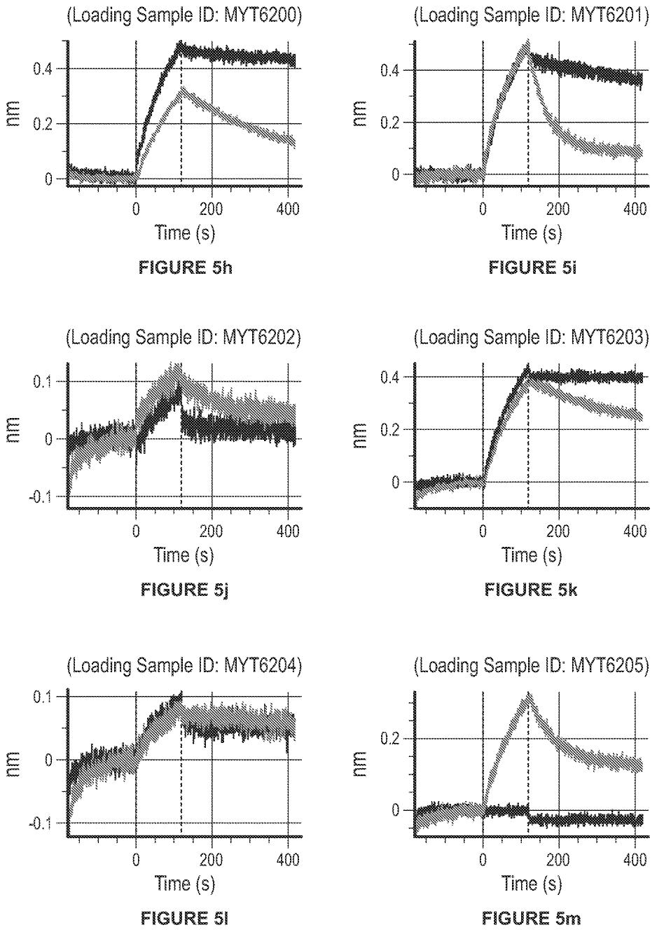

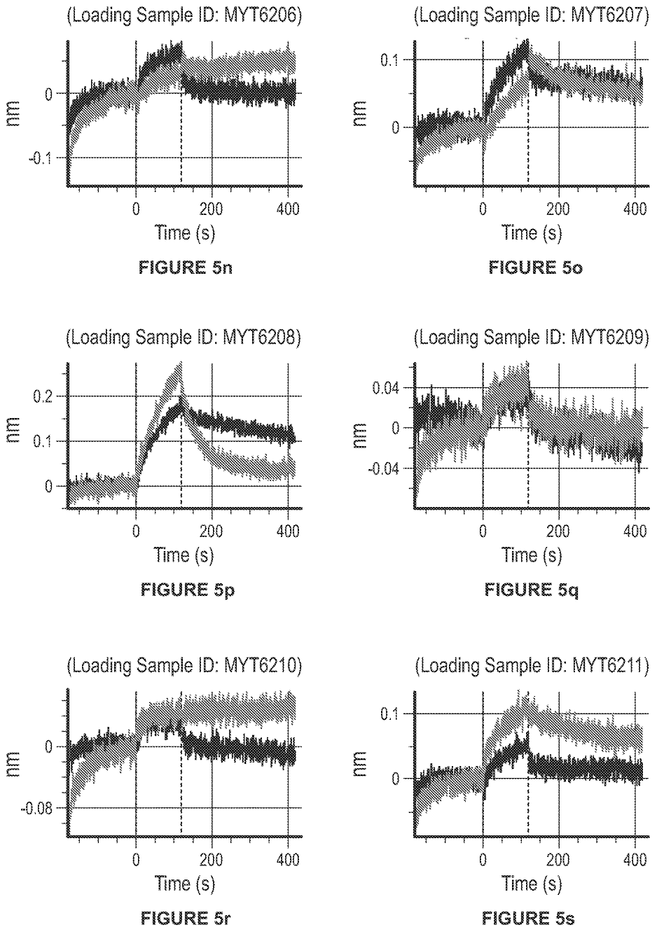

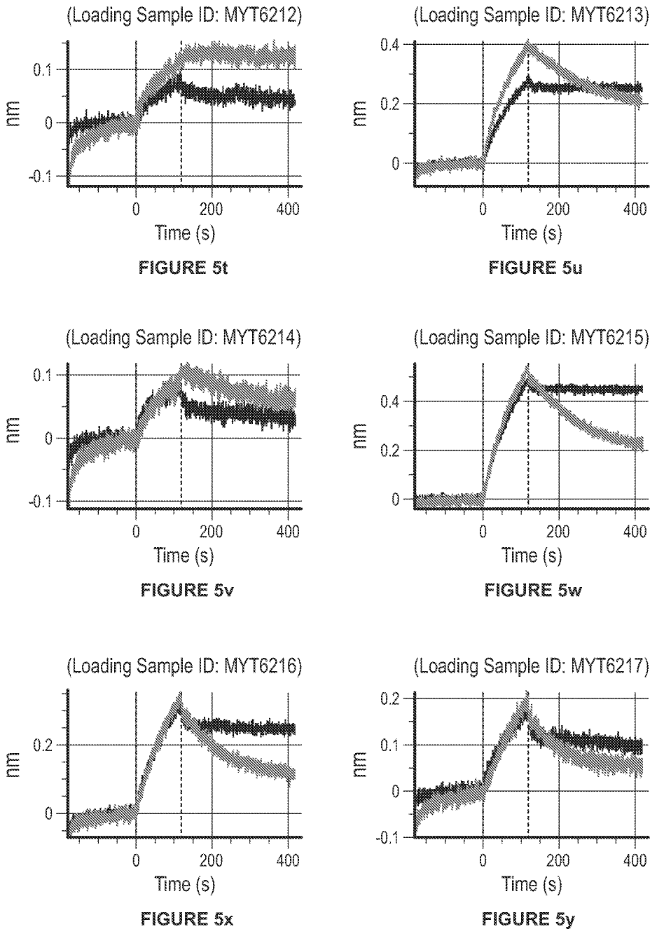

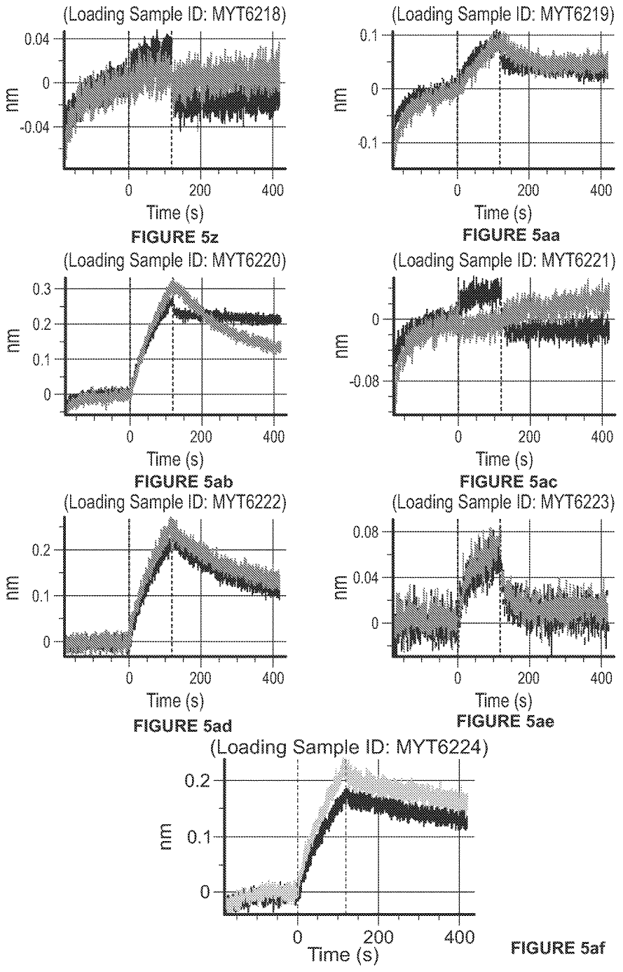

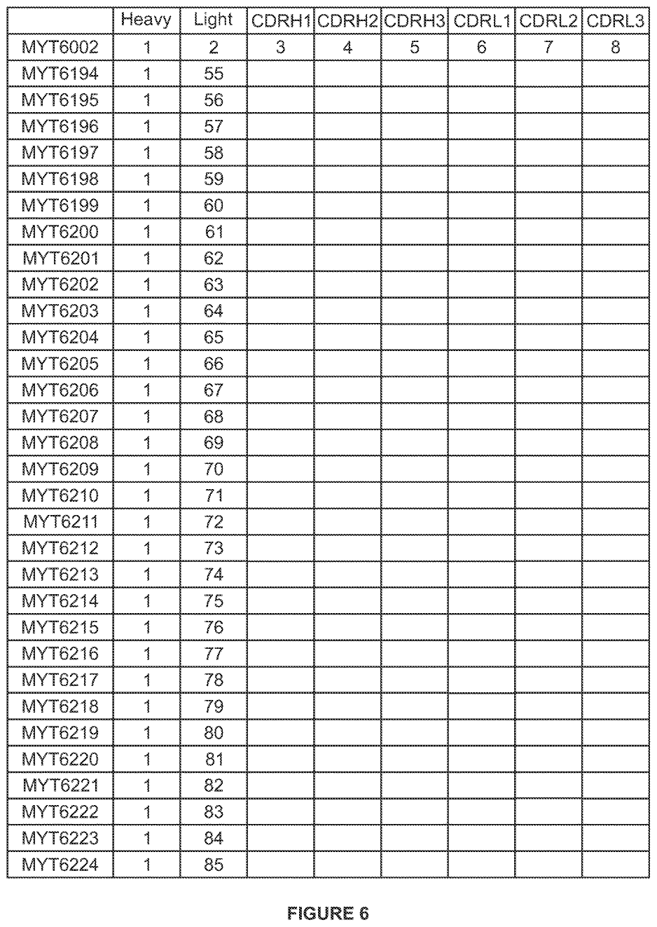

Multiple PTK7-binding monoclonal antibodies have been described in the literature and can be used as a template for engineering pH-dependent binding [Damelin et. al., A PTK7-Targeted Antibody-Drug Conjugate Reduces Tumor-Initiating Cells and Induces Sustained Tumor Regressions, Sci Transl Med. 2017 Jan. 11; 9 (372): eaag2611.]. We selected cofetuzumab (Heavy chain SEQ ID NO: 1, Light chain SEQ ID NO: 2) as a PTK7-binding monoclonal antibody for pH engineering via histidine scanning. Briefly, CDRs in the light chain were identified using the methods described by Kabat et al (Kabat et al. (1992) Sequences of Proteins of Immunological Interest, DIANE publishing) and IMGT (Lefranc MP (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the light chain CDRs were systematically substituted with a histidine, one at a time (MYT6194-MYT6224). In cases where the starting CDR residue was a histidine, it was mutated to an alanine. Antibody variants with only one histidine or alanine mutation in a light chain CDR were generated by co-transfection of Expi293 cells with a) one light chain sequence variant, and b) the corresponding starting ABPC heavy chain using methods known to the art. After allowing for four days of protein expression, cell culture supernatants were collected, quantified by SDS-PAGE analysis (FIG. 4), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20) pH 7.4, and the sensor was associated with 50 nM of PTK7 (Human PTK7/CCK4 Protein, His Tag, Acro Biosystems PT7-H52H3, Lot No. 4313a-212BF1-VH) in 1×PBST pH 7.4 for 120 sec to generate an association curve. In the dissociation phase, the antibody-antigen complex on the sensor was exposed to 1×PBST pH 7.4 or pH 5.4 for 300 sec. Association and dissociation phase curves were examined for the starting ABPC antibody (with no substitutions) and each corresponding antibody variant at pH 5.4 and pH 7.4 to inform on two criteria: a) enhanced dissociation (i.e., higher koff values) at pH 5.4 due to histidine or alanine substitution compared to the starting ABPC (with no substitutions), and b) reduced dissociation at pH 7.4 (i.e., lower koff values) compared to pH 5.4 in the antibody variant itself and with the starting ABPC (with no substitutions). Light chain variants that showed either enhanced dissociation at pH 5.4 or reduced dissociation at pH 7.4 or both (as compared to the starting ABPC), as shown in FIG. 5 were selected for further analysis (e.g., MYT6195, MYT6199, MYT6201, MYT6205, MYT6208, MYT6215, MYT6216, and MYT6217). It was also noted that while some histidine and alanine mutations obliterated PTK7 binding (e.g., MYT6206, MYT6209, MYT6210, MYT6218, and MYT6221), others were tolerated with little (e.g., less than 1-fold change in dissociation constant KD or dissociation rate) or no change in PTK7 binding kinetics (e.g., MYT6194, MYT6196, MYT6197, MYT6198, MYT6200, MYT6203, MYT6213, and MYT6220).

Especially because histidine is a large, positively charged amino acid, these variants with no change were noted as positions in the light chain that may tolerate a wide range of mutations and lead to antibodies with different sequence but similar binding properties, a designation that is not otherwise apparent.

### Example 11. Construction and Screening of pH-Engineered PTK7 ABPCs

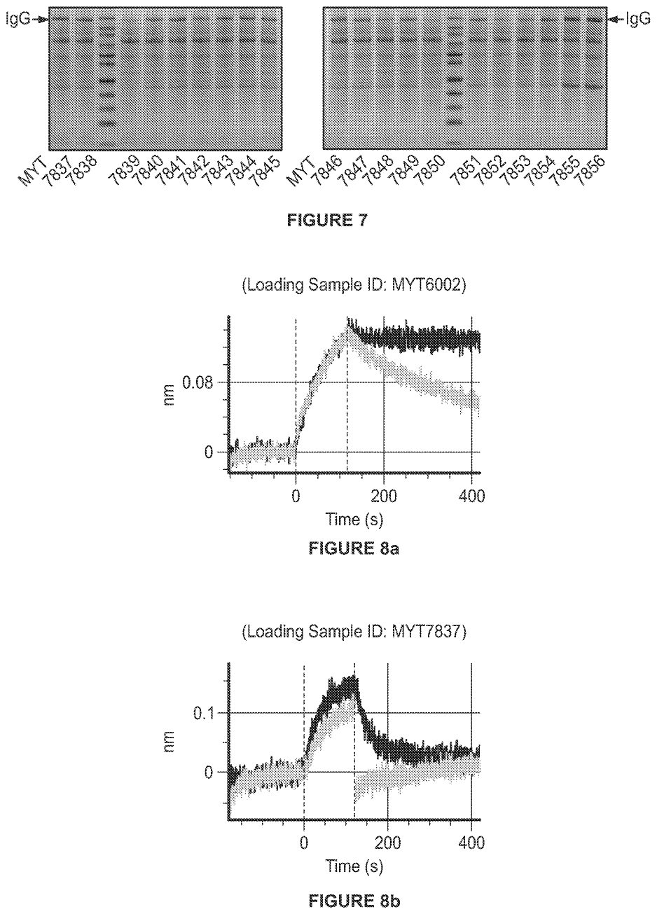

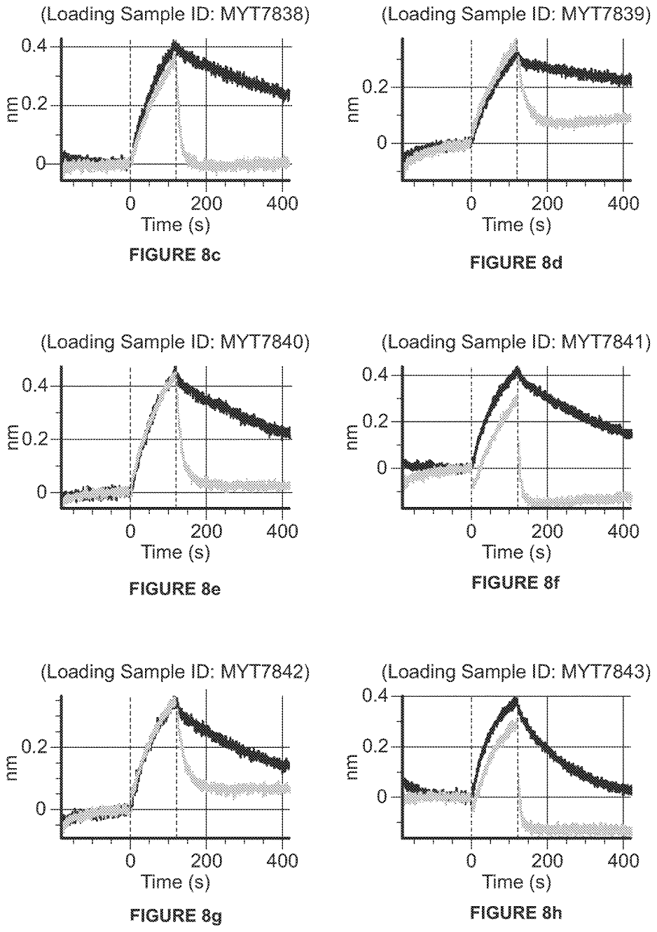

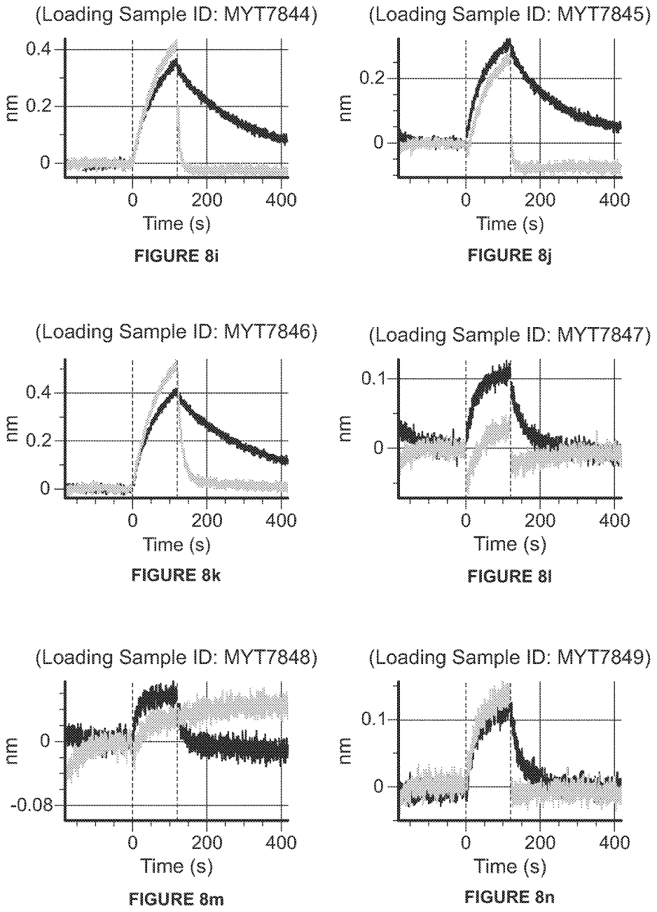

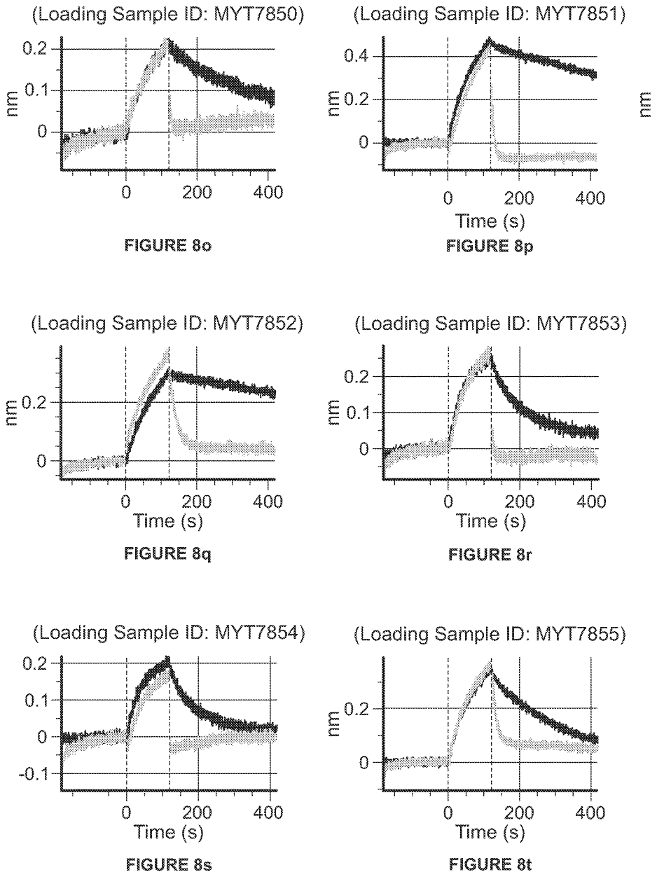

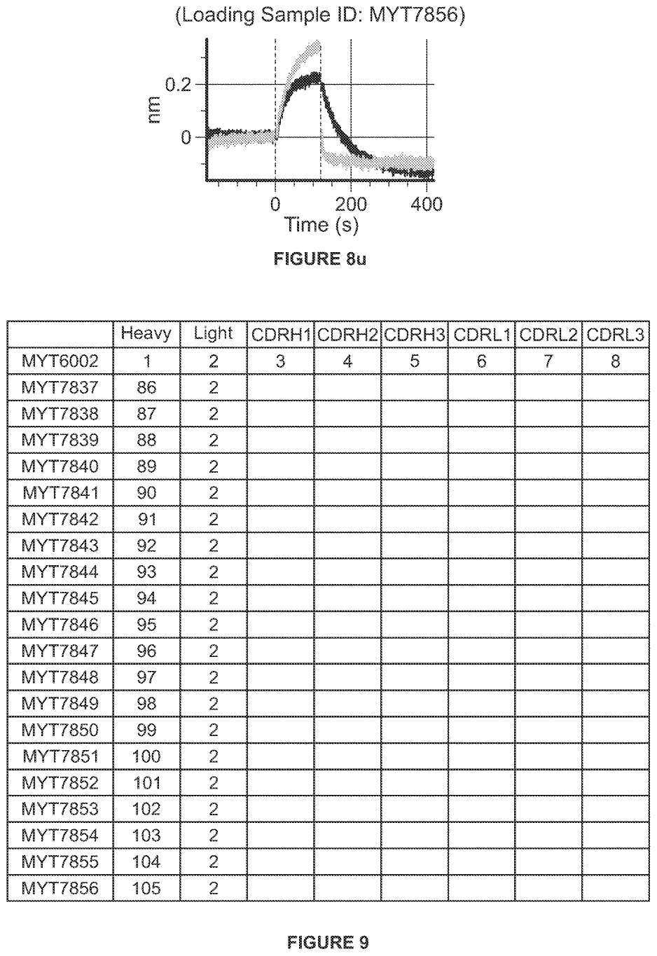

Multiple PTK7-binding monoclonal antibodies have been described in the literature and can be used as a template for engineering pH-dependent binding [Damelin et. al., A PTK7-Targeted Antibody-Drug Conjugate Reduces Tumor-Initiating Cells and Induces Sustained Tumor Regressions, Sci Transl Med. 2017 Jan. 11; 9 (372): eaag2611.]. We selected cofetuzumab (Heavy chain SEQ ID NO: 1, Light chain SEQ ID NO: 2) as a PTK7-binding monoclonal antibody for pH engineering via histidine scanning. Briefly, CDRs in the heavy chain were identified using the methods described by Kabat et al (Kabat et al. (1992) Sequences of Proteins of Immunological Interest, DIANE publishing) and IMGT (Lefranc MP (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid mutations within the heavy chain CDRs that had been previously selected for further analysis in Example 9 were systematically combined two or more at a time (MYT7837-MYT7856). In cases where the starting CDR residue was a histidine, it was mutated to an alanine. Antibody variants with two or more histidine or alanine mutations in the heavy chain CDRs were generated by co-transfection of Expi293 cells with a) one heavy chain combinations sequence variant, and b) the corresponding starting ABPC light chain using methods known to the art. After allowing for four days of protein expression, cell culture supernatants were collected, quantified by SDS-PAGE analysis (FIG. 7), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20) pH 7.4, and the sensor was associated with 50 nM of PTK7 (Recombinant human CCK4/PTK7 Protein (His Tag), Sino Biological 19399-H08H, Lot No. MB14AP1271) in 1×PBST pH 7.4 for 120 sec to generate an association curve. In the dissociation phase, the antibody-antigen complex on the sensor was exposed to 1×PBST pH 7.4 or pH 5.4 for 300 sec. Association and dissociation phase curves were examined for the starting ABPC antibody (with no substitutions) and each corresponding antibody variant at pH 5.4 and pH 7.4 to inform on two criteria: a) enhanced dissociation (i.e., higher koff values) at pH 5.4 due to histidine or alanine substitution compared to the starting ABPC (with no substitutions), and b) reduced dissociation at pH 7.4 (i.e., lower koff values) compared to pH 5.4 in the antibody variant itself and with the starting ABPC (with no substitutions). Heavy chain combinations variants that showed either enhanced dissociation at pH 5.4 or reduced dissociation at pH 7.4 or both (as compared to the starting ABPC), as shown in FIG. 8 were selected for further analysis (e.g., MYT7837, MYT7838, MYT7839, MYT7840, MYT7841, MYT7842, MYT7843, MYT7844, MYT7845, MYT7846, MYT7847, MYT7849, MYT7850, MYT7851, MYT7852, MYT7853, MYT7854, MYT7855, and MYT7856).

### Example 12. Construction and Screening of pH-Engineered PTK7 ABPCs

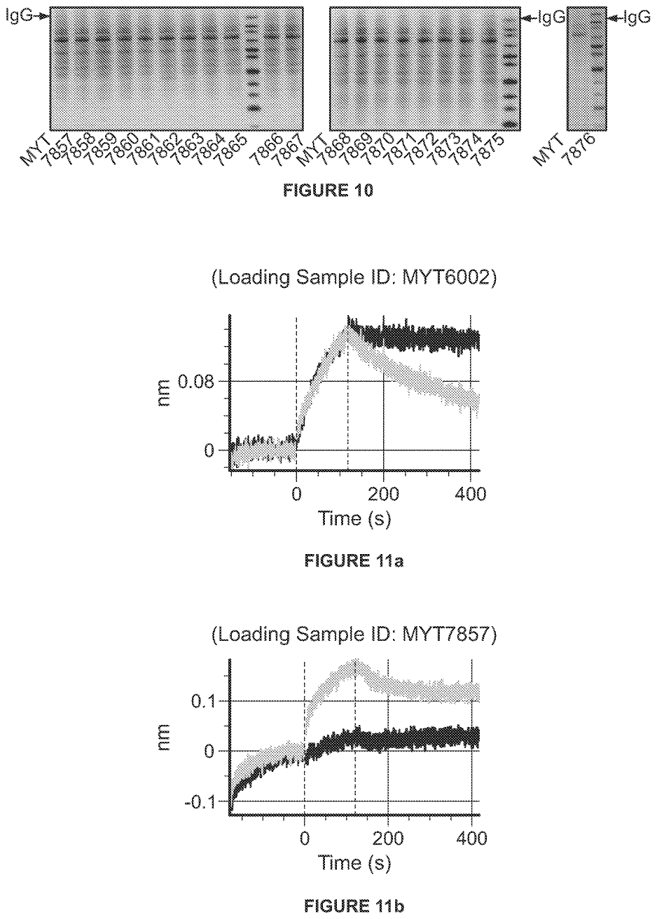

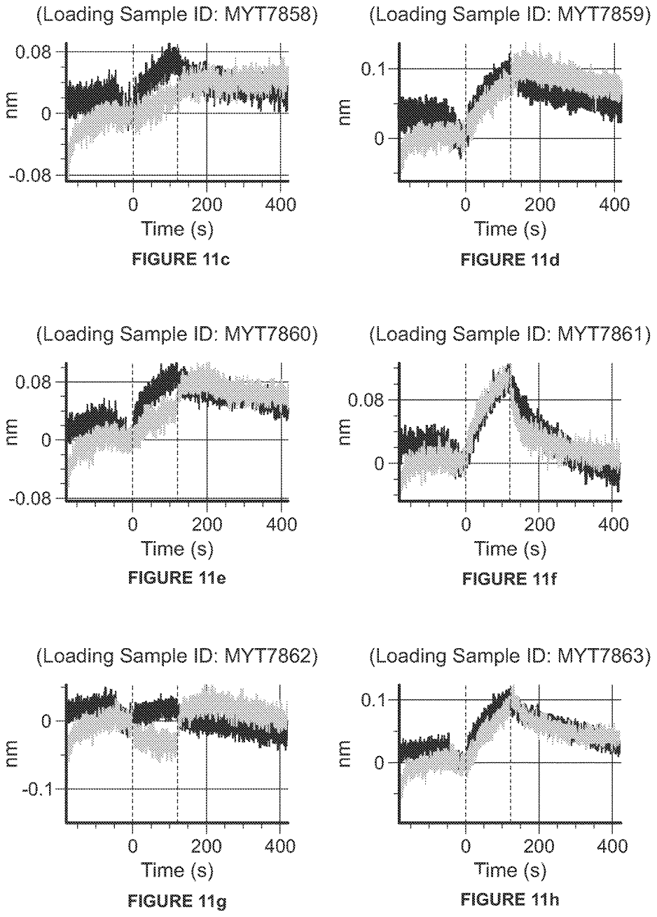

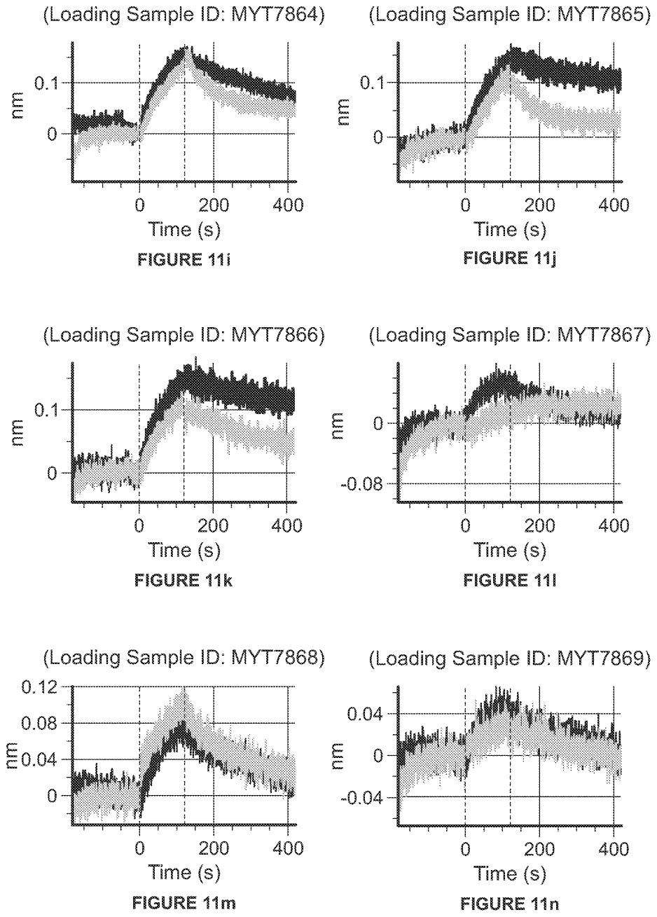

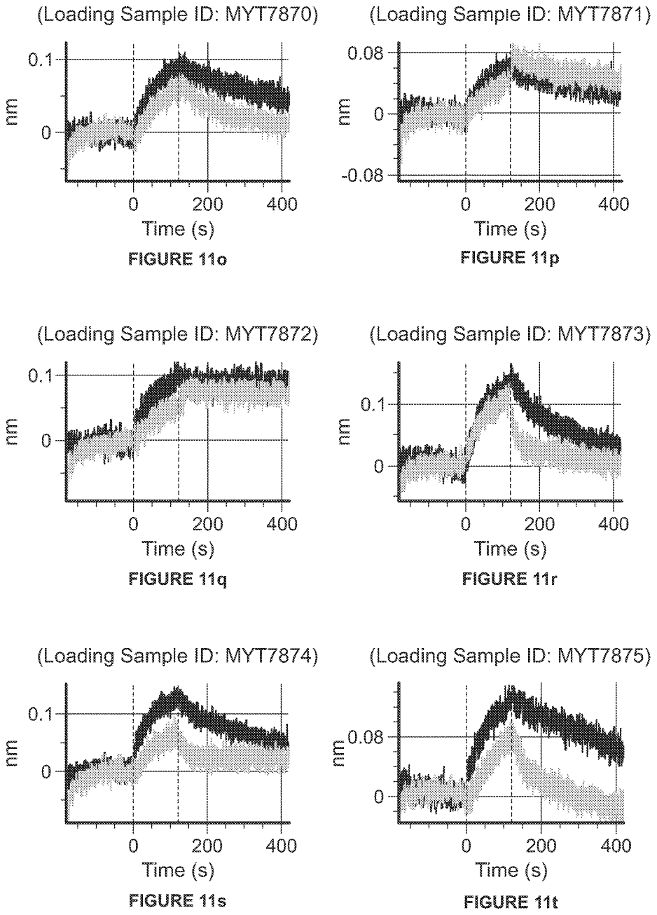

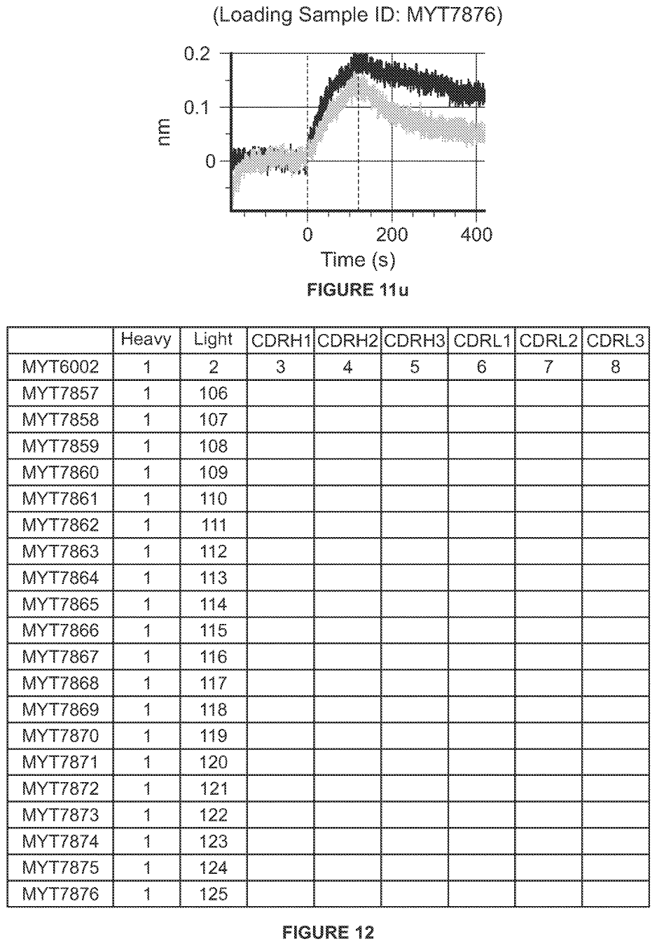

Multiple PTK7-binding monoclonal antibodies have been described in the literature and can be used as a template for engineering pH-dependent binding [Damelin et. al., A PTK7-Targeted Antibody-Drug Conjugate Reduces Tumor-Initiating Cells and Induces Sustained Tumor Regressions, Sci Transl Med. 2017 Jan. 11; 9 (372): eaag2611.]. We selected cofetuzumab (Heavy chain SEQ ID NO: 1, Light chain SEQ ID NO: 2) as a PTK7-binding monoclonal antibody for pH engineering via histidine scanning. Briefly, CDRs in the light chain were identified using the methods described by Kabat et al (Kabat et al. (1992) Sequences of Proteins of Immunological Interest, DIANE publishing) and IMGT (Lefranc MP (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid mutations within the light chain CDRs that had been previously selected for further analysis in Example 10 were systematically combined two or more at a time (MYT7857-MYT7876). In cases where the starting CDR residue was a histidine, it was mutated to an alanine. Antibody variants with two or more histidine or alanine mutations in the light chain CDRs were generated by co-transfection of Expi293 cells with a) one light chain combinations sequence variant, and b) the corresponding starting ABPC heavy chain using methods known to the art. After allowing for four days of protein expression, cell culture supernatants were collected, quantified by SDS-PAGE analysis (FIG. 10), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM PTK7 (Recombinant human CCK4/PTK7 Protein (His Tag), Sino Biological 19399-H08H, Lot No. MB14AP1271) in 1×PBST, pH 7.4, for 120 sec to generate an association curve. In the dissociation phase, the antibody-antigen complex on the sensor was exposed to 1×PBST, pH 7.4 or pH 5.4, for 300 sec. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Light chain combinations variants that showed either enhanced dissociation at pH 5.4 or reduced dissociation at pH 7.4 or both (as compared to the starting ABPC), as shown in FIG. 11 were selected for further analysis (e.g., MYT7861, MYT7864, MYT7865, MYT7870, MYT7873, MYT7874, MYT7875, and MYT7876). It was also noted that some light chain combination histidine and alanine scanning variants obliterated PTK7 binding (e.g. MYT7858 and MYT7862).

### Example 13. Construction and Screening of pH-Engineered PTK7 ABPCs

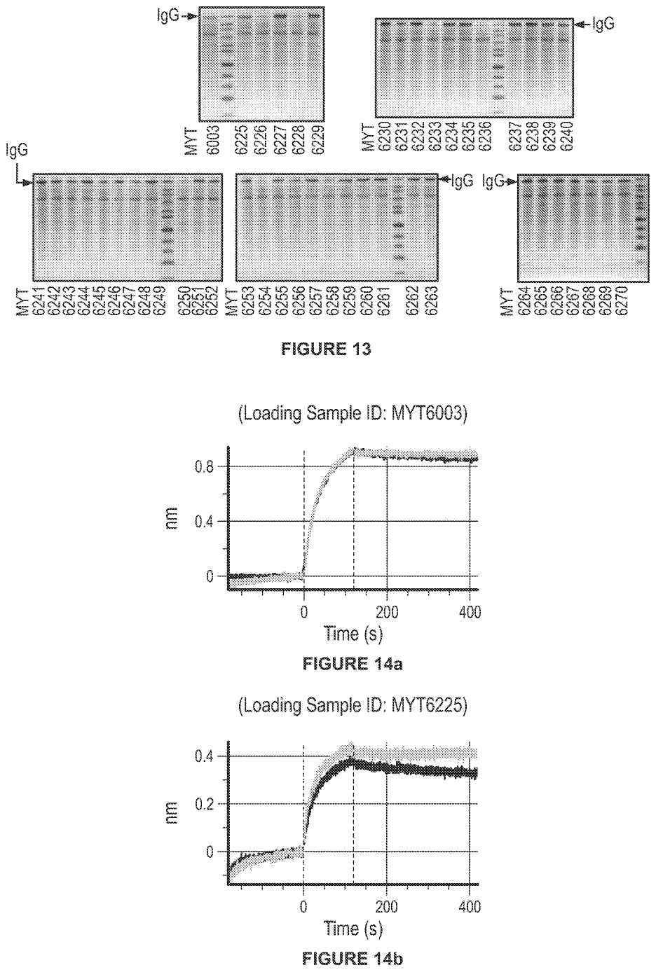

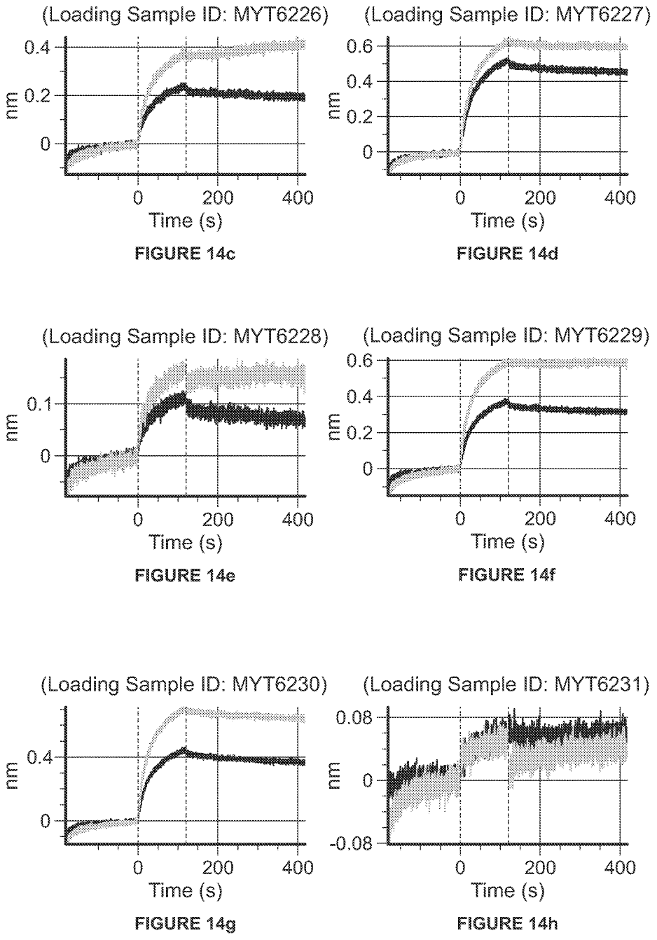

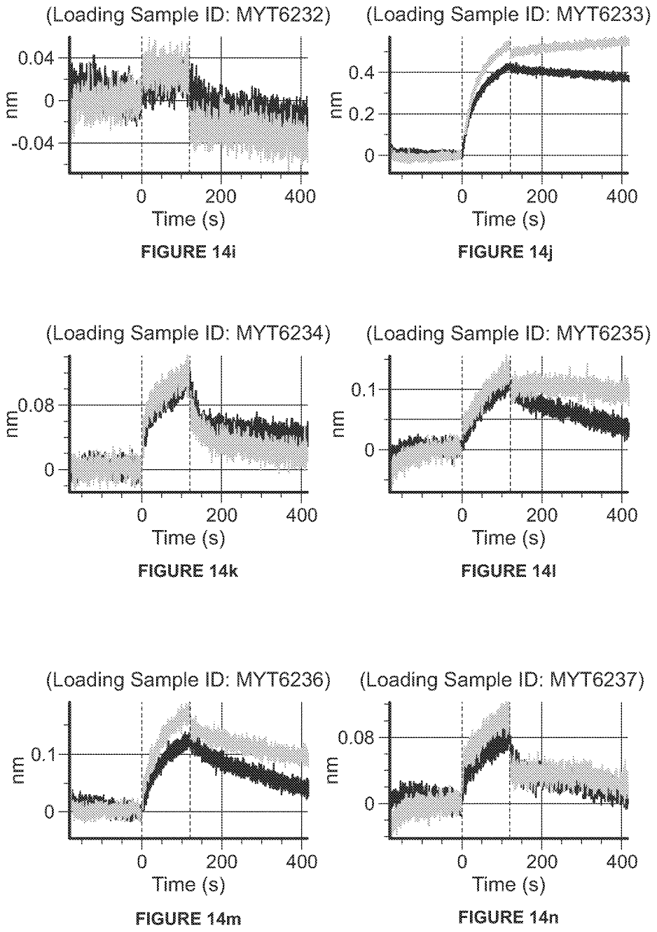

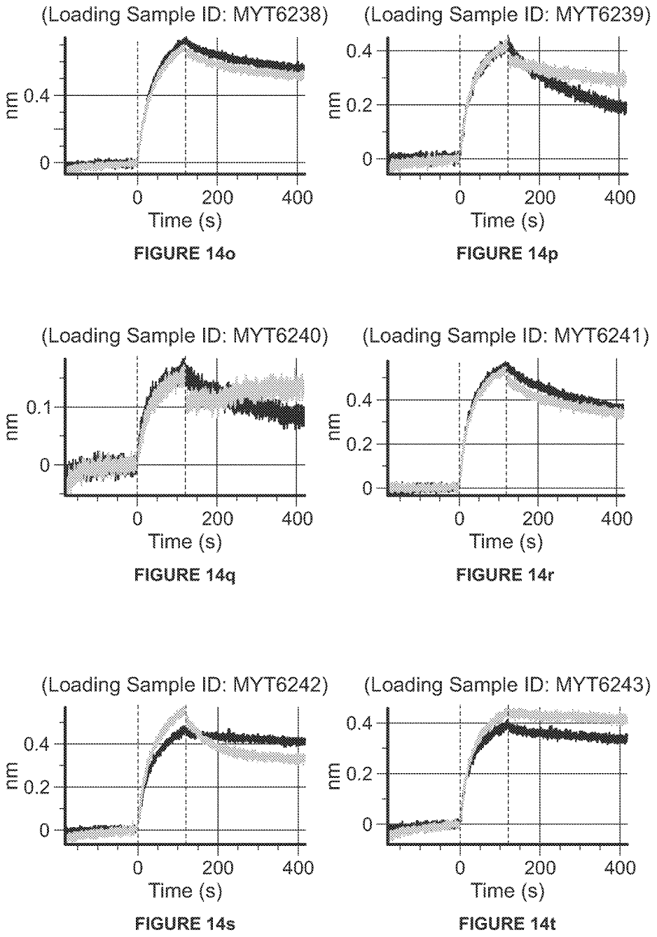

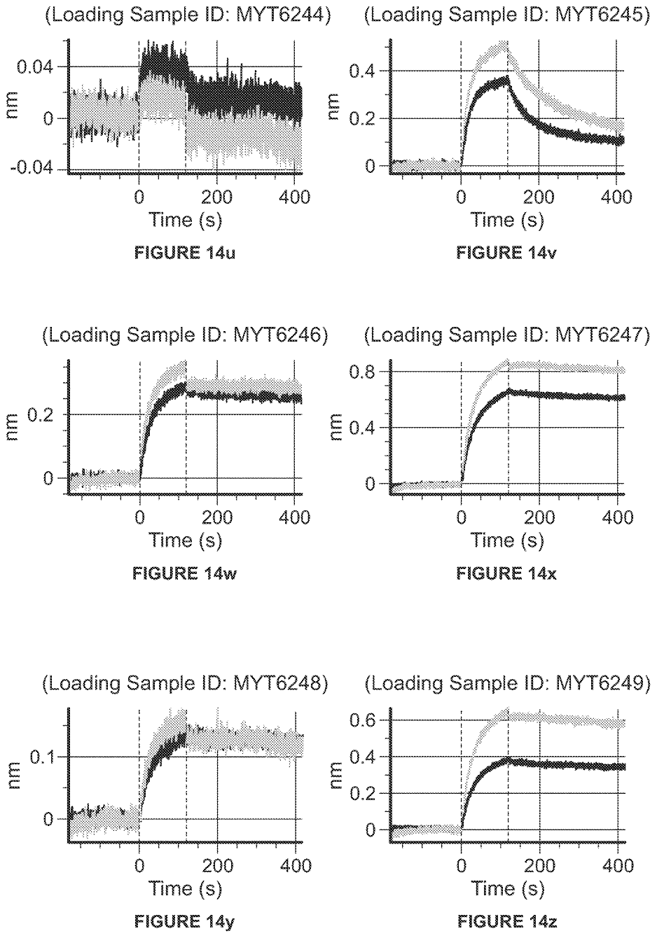

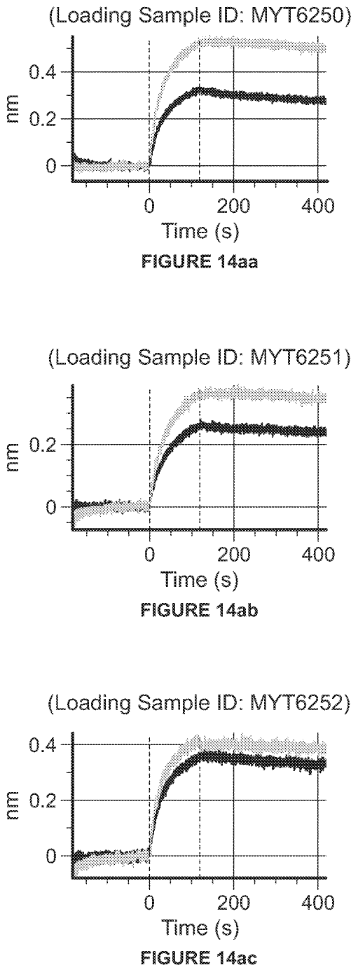

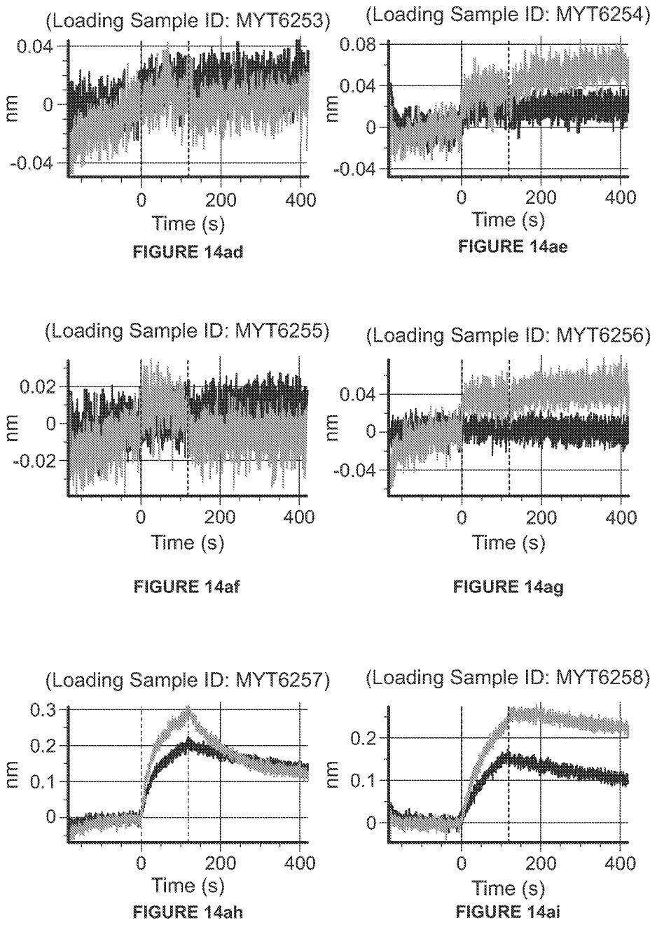

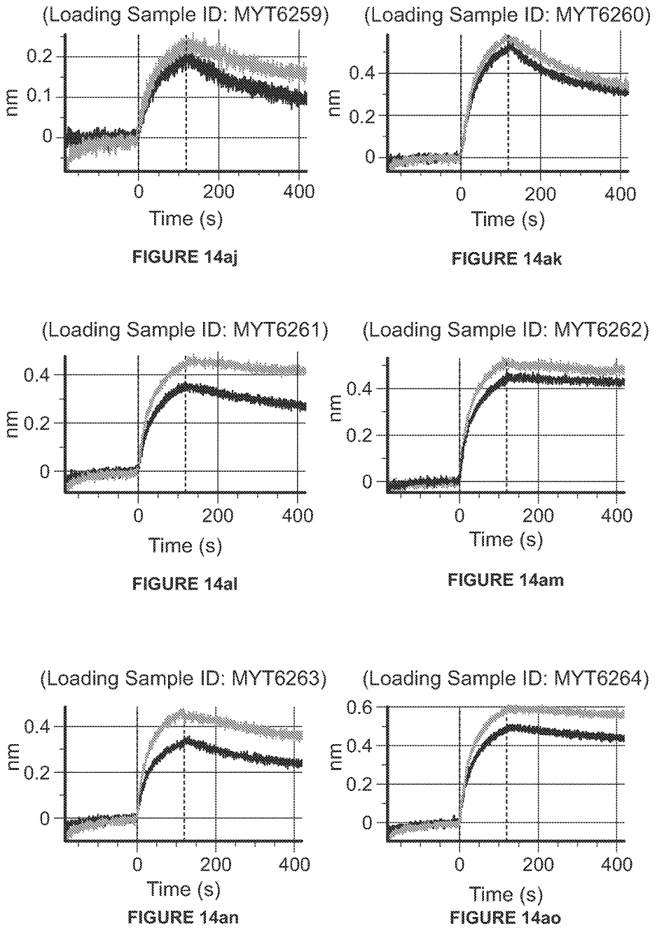

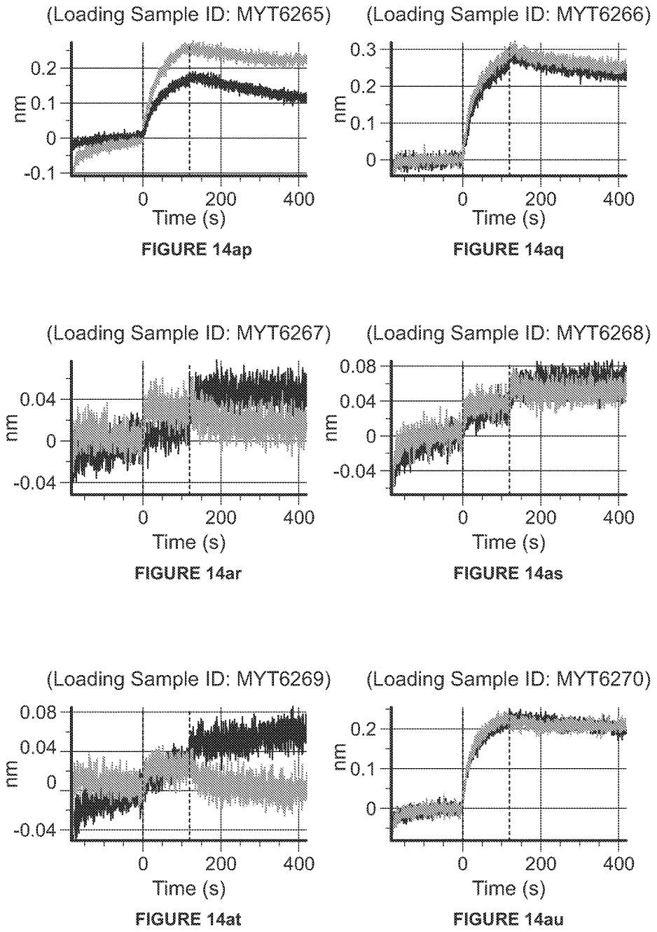

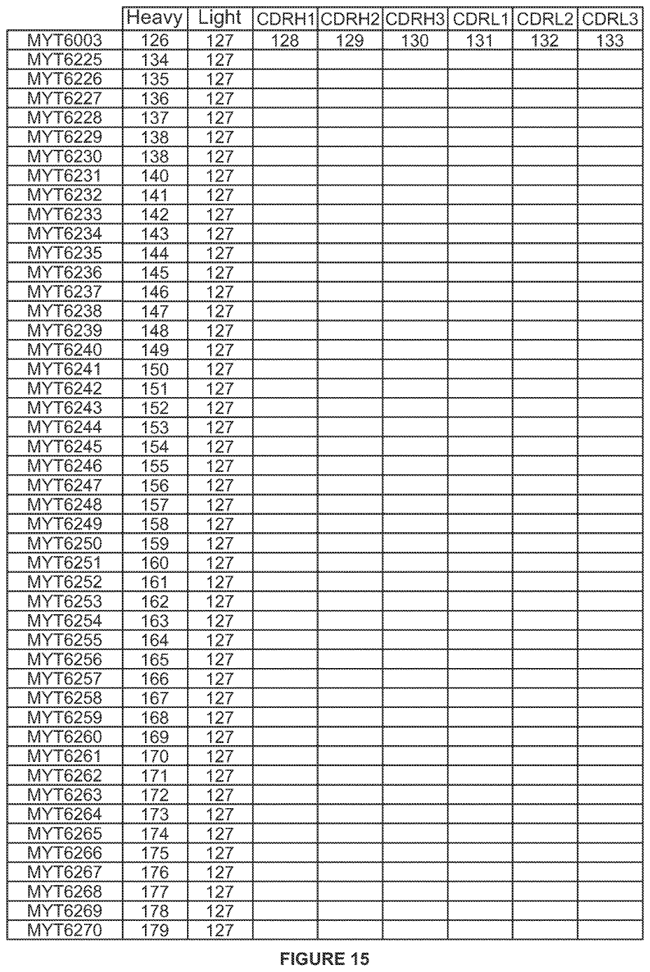

Multiple PTK7-binding monoclonal antibodies have been described in the literature and can be used as a template for engineering pH-dependent binding [Terrett, J et al. “Human Monoclonal Antibodies to Protein Tyrosine Kinase 7 (PTK7)”, U.S. Pat. No. 9,102,738 B2 (2015)]. We selected 7C8 (Heavy chain SEQ ID NO: 126, Light chain SEQ ID NO: 127) as a PTK7-binding monoclonal antibody for pH engineering via histidine scanning. Briefly, CDRs in the heavy chain were identified using the methods described by Kabat et al (Kabat et al. (1992) Sequences of Proteins of Immunological Interest, DIANE publishing) and IMGT (Lefranc MP (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the heavy chain CDRs were systematically substituted with a histidine, one at a time (MYT6225-MYT6270). In cases where the starting CDR residue was a histidine, it was mutated to an alanine. Antibody variants with only one histidine or alanine mutation in a heavy chain CDR were generated by co-transfection of Expi293 cells with a) one heavy chain sequence variant, and b) the corresponding starting ABPC light chain using methods known to the art. After allowing for four days of protein expression, cell culture supernatants were collected, quantified by SDS-PAGE analysis (FIG. 13), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. A baseline was established using 1×PBST (50 mM Potassium Phosphate Buffer+150 mM NaCl+0.05% Tween 20) pH 7.4, and the sensor was associated with 50 nM of PTK7 (Human PTK7/CCK4 Protein, His Tag, Acro Biosystems PT7-H52H3, Lot No. 4313a-212BF1-VH) in 1×PBST pH 7.4 for 120 sec to generate an association curve. In the dissociation phase, the antibody-antigen complex on the sensor was exposed to 1×PBST pH 7.4 or pH 5.4 for 300 sec. Association and dissociation phase curves were examined for the starting ABPC antibody (with no substitutions) and each corresponding antibody variant at pH 5.4 and pH 7.4 to inform on two criteria: a) enhanced dissociation (i.e., higher koff values) at pH 5.4 due to histidine or alanine substitution compared to the starting ABPC, (with no substitutions), and b) reduced dissociation at pH 7.4 (i.e., lower koff values) compared to pH 5.4 in the antibody variant itself and with the starting ABPC (with no substitutions). Heavy chain variants that showed either enhanced dissociation at pH 5.4 or reduced dissociation at pH 7.4 or both (as compared to starting ABPC) as shown in FIG. 14 were selected for further analysis (e.g., MYT6238, MYT6239, MYT6241, MYT6242, MYT6245, MYT6257, MYT6258, MYT6259, MYT6260, MYT6261, MYT6263, MYT6264, MYT6265, and MYT6266). It was also noted that while some histidine and alanine mutations obliterated PTK7 binding (e.g., MYT6231, MYT6232, MYT6244, MYT6253, MYT6254, MYT6255, MYT6256, MYT6267, MYT6268, and MYT6269), others were tolerated with little (e.g., less than 1-fold change in dissociation constant KD or dissociation rate) or no change in PTK7 binding kinetics (e.g., MYT6225, MYT6226, MYT6227, MYT6228, MYT6229, MYT6230, MYT6233, MYT6243, MYT6246, MYT6247, MYT6248, MYT6249, MYT6250, MYT6251, MYT6252, MYT6262, and MYT6270).

Especially because histidine is a large, positively charged amino acid, these variants with no change were noted as positions in the heavy chain that may tolerate a wide range of mutations and lead to antibodies with different sequence but similar binding properties, a designation that is not otherwise apparent.

### Example 14. Construction and Screening of pH-Engineered PTK7 ABPCs

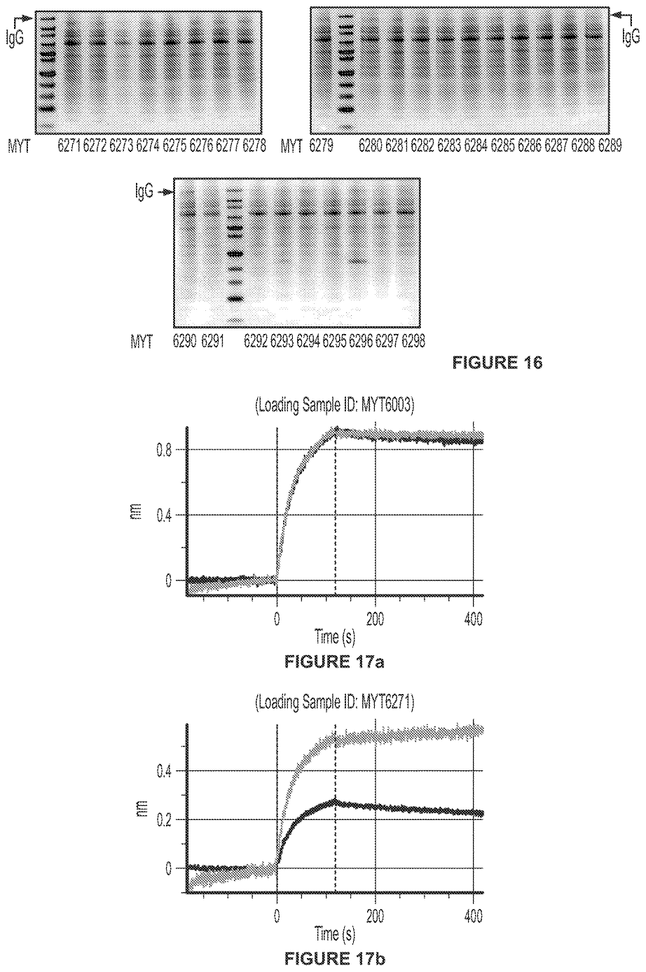

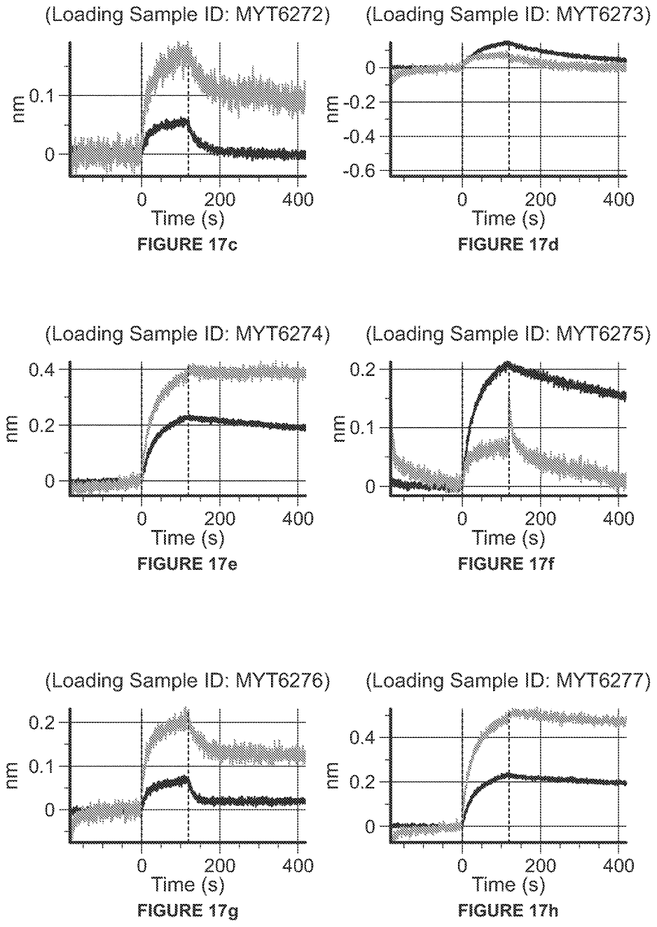

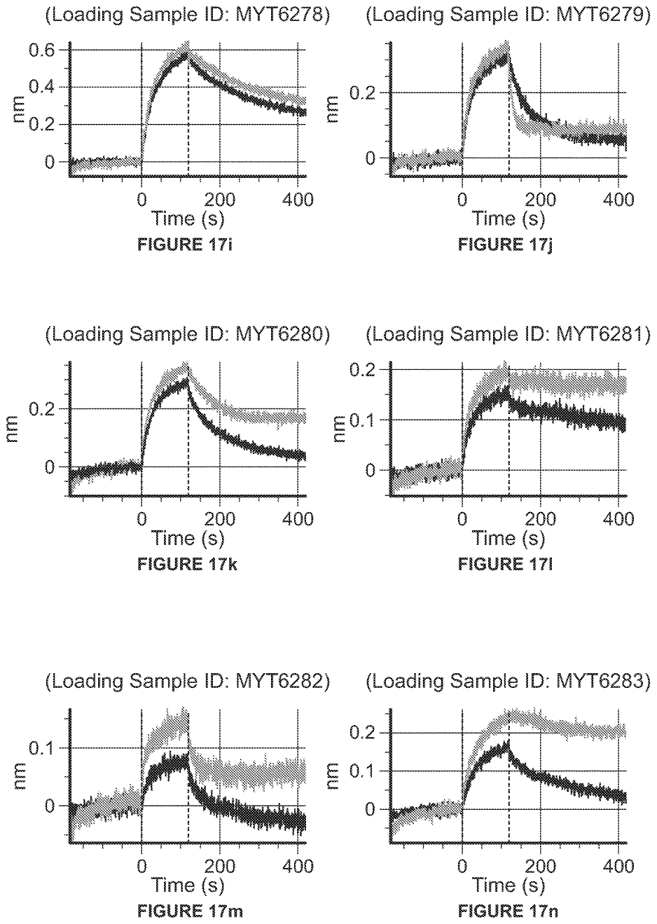

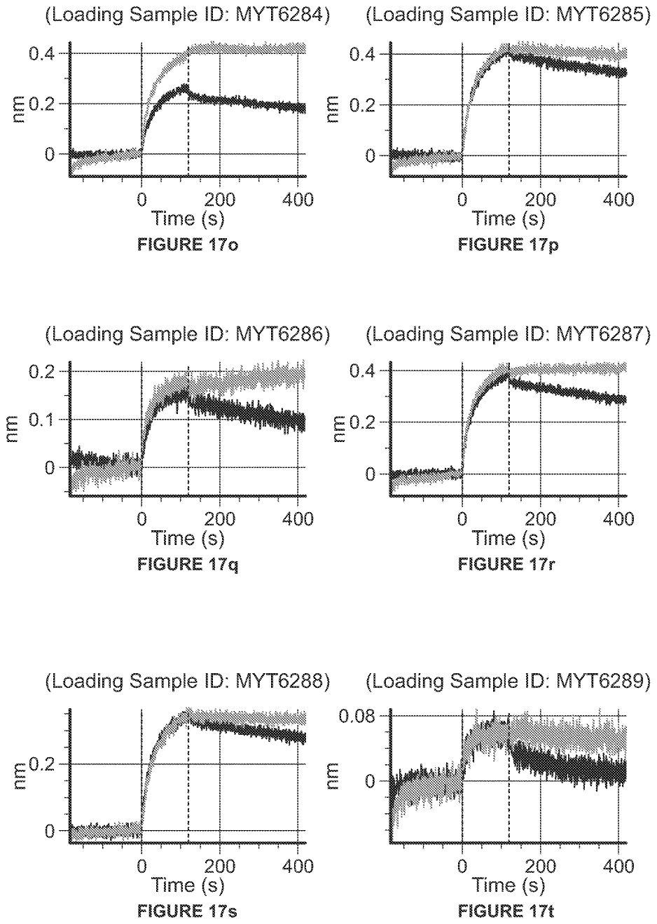

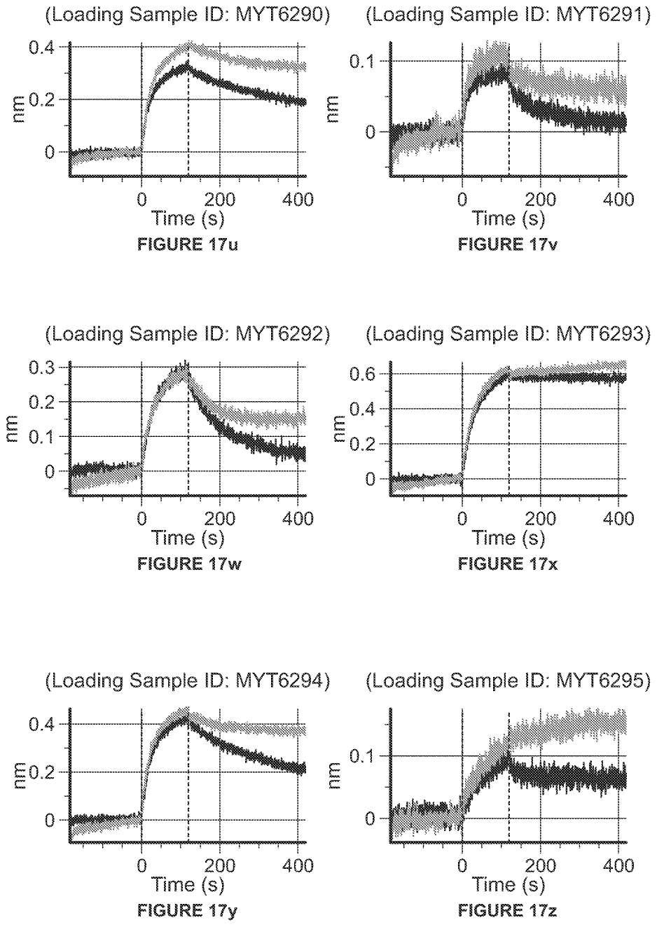

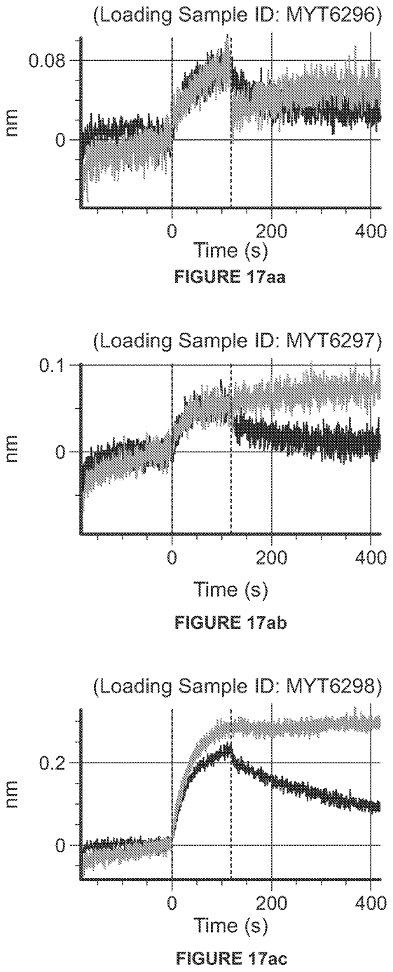

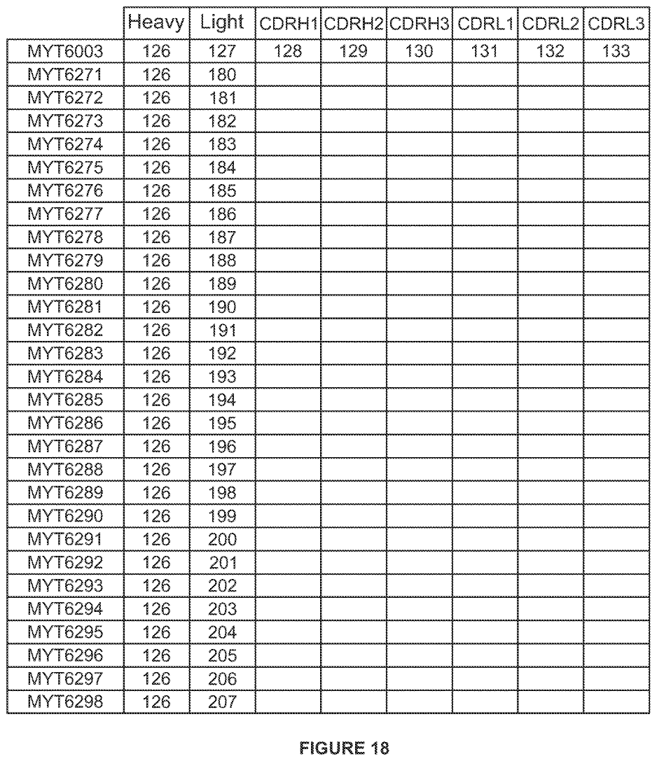

Multiple PTK7-binding monoclonal antibodies have been described in the literature and can be used as a template for engineering pH-dependent binding [Terrett, J et al. “Human Monoclonal Antibodies to Protein Tyrosine Kinase 7 (PTK7)”, U.S. Pat. No. 9,102,738 B2 (2015)]. We selected 7C8 (Heavy chain SEQ ID NO: 126, Light chain SEQ ID NO: 127) as a PTK7-binding monoclonal antibody for pH engineering via histidine scanning. Briefly, CDRs in the light chain were identified using the methods described by Kabat et al (Kabat et al. (1992) Sequences of Proteins of Immunological Interest, DIANE publishing) and IMGT (Lefranc MP (1999) “The IMGT unique numbering for Immunoglobulins, T cell receptors and Ig-like domains” The Immunologist 7, 132-136), and for each CDR, residues falling under cither or both Kabat and IMGT CDR definitions were called as CDR residues. To generate pH-dependent sequence variants, individual amino acid residues within the light chain CDRs were systematically substituted with a histidine, one at a time (MYT6271-MYT6298). In cases where the starting CDR residue was a histidine, it was mutated to an alanine. Antibody variants with only one histidine or alanine mutation in a light chain CDR were generated by co-transfection of Expi293 cells with a) one light chain sequence variant, and b) the corresponding starting ABPC heavy chain using methods known to the art. After allowing for four days of protein expression, cell culture supernatants were collected, quantified by SDS-PAGE analysis (FIG. 16), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20) pH 7.4, and the sensor was associated with 50 nM of PTK7 (Human PTK7/CCK4 Protein, His Tag, Acro Biosystems PT7-H52H3, Lot No. 4313a-212BF1-VH) in 1×PBST pH 7.4 for 120 sec to generate an association curve. In the dissociation phase, the antibody-antigen complex on the sensor was exposed to 1×PBST pH 7.4 or pH 5.4 for 300 sec. Association and dissociation phase curves were examined for the starting ABPC antibody (with no substitutions) and each corresponding antibody variant at pH 5.4 and pH 7.4 to inform on two criteria: a) enhanced dissociation (i.e., higher koff values) at pH 5.4 due to histidine or alanine substitution compared to the starting ABPC (with no substitutions), and b) reduced dissociation at pH 7.4 (i.e., lower koff values) compared to pH 5.4 in the antibody variant itself and with the starting ABPC (with no substitutions). Light chain variants that showed either enhanced dissociation at pH 5.4 or reduced dissociation at pH 7.4 or both (as compared to the starting ABPC), as shown in FIG. 17 were selected for further analysis (e.g., MYT6272, MYT6273, MYT6275, MYT6276, MYT6278, MYT6279, MYT6280, MYT6282, MYT6290, MYT6292, and MYT6294). It was also noted that some histidine and alanine mutations were tolerated with little (e.g., less than 1-fold change in dissociation constant KD or dissociation rate) or no change in PTK7 binding kinetics (e.g., MYT6271, MYT6274, MYT6277, MYT6284, and MYT6293).

Especially because histidine is a large, positively charged amino acid, these variants with no change were noted as positions in the light chain that may tolerate a wide range of mutations and lead to antibodies with different sequence but similar binding properties, a designation that is not otherwise apparent.

### Example 15. Construction and Screening of pH-Engineered PTK7 ABPCs

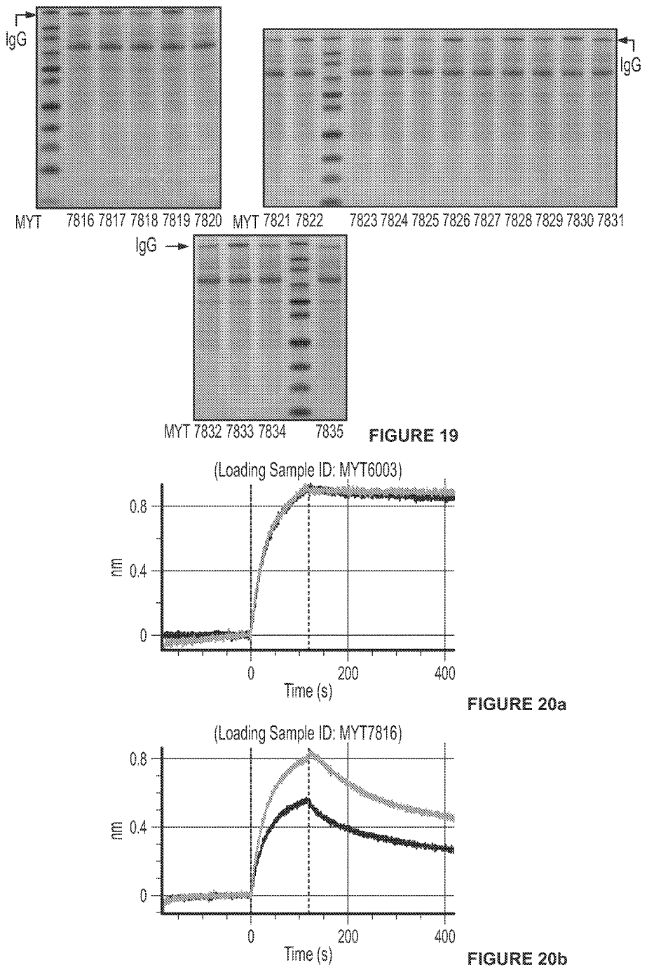

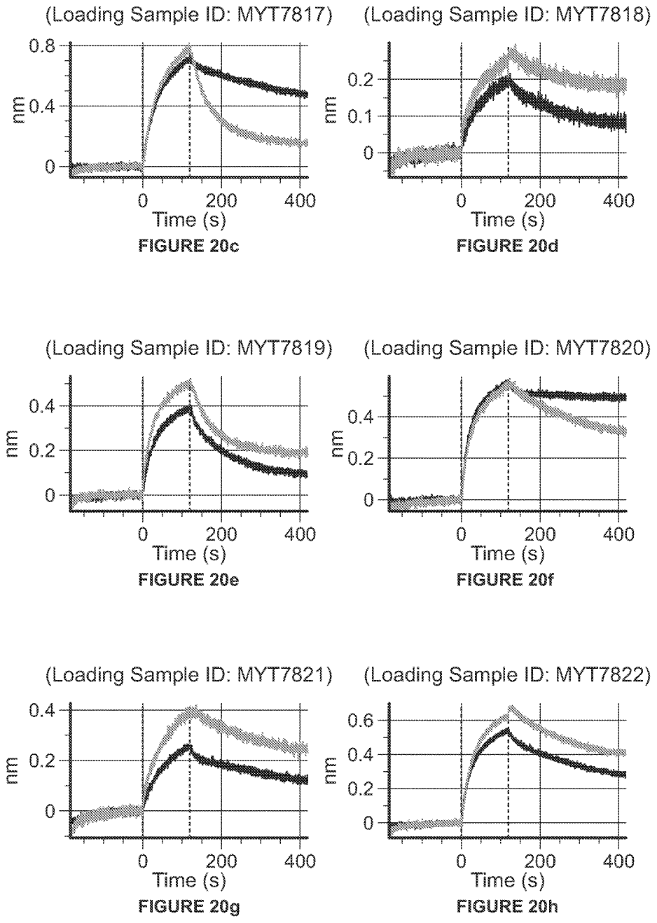

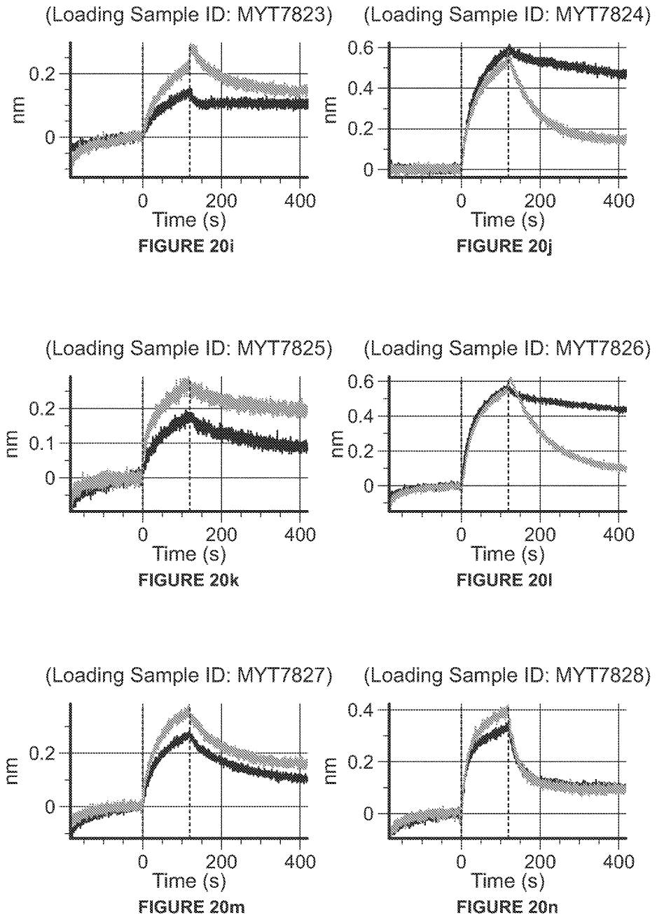

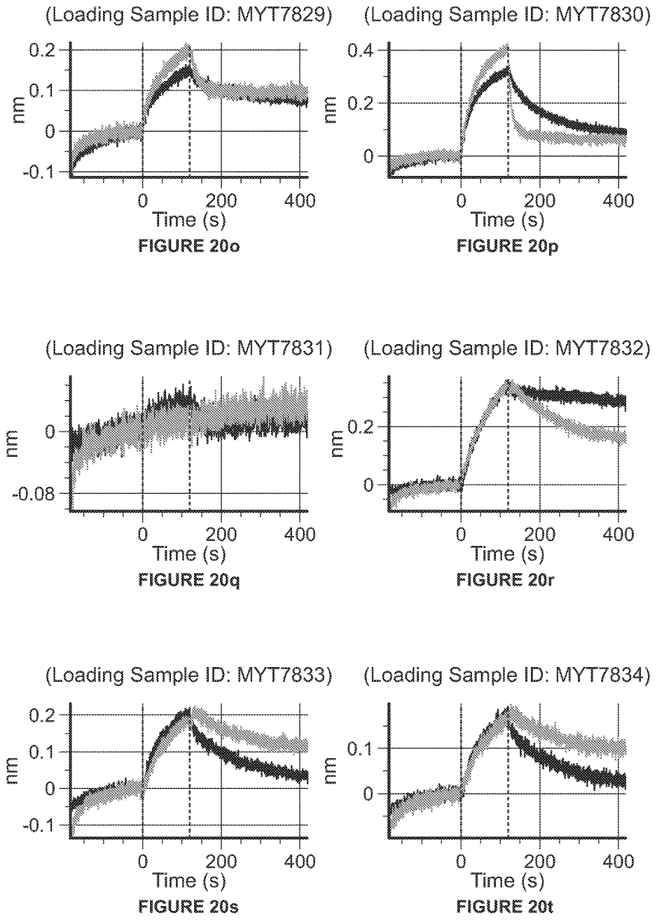

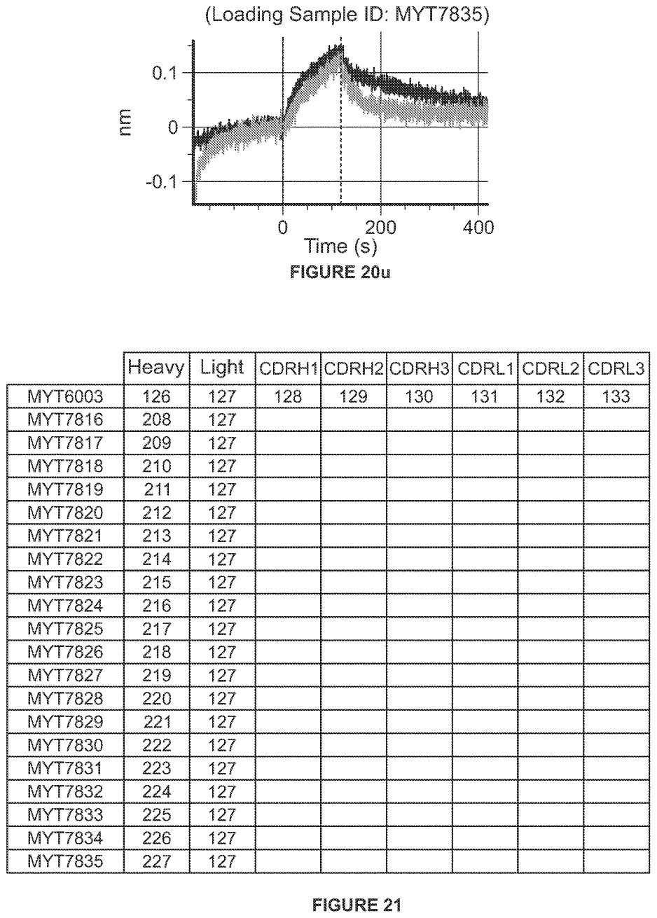

Multiple PTK7-binding monoclonal antibodies have been described in the literature and can be used as a template for engineering pH-dependent binding [Terrett, J et al. “Human Monoclonal Antibodies to Protein Tyrosine Kinase 7 (PTK7)”, U.S. Pat. No. 9,102,738 B2 (2015)]. We selected 7C8 (Heavy chain SEQ ID NO: 126, Light chain SEQ ID NO: 127) as a PTK7-binding monoclonal antibody for pH engineering via histidine scanning. Briefly, CDRs in the heavy chain were identified using the methods described by Kabat et al (Kabat et al. (1992) Sequences of Proteins of Immunological Interest, DIANE publishing) and IMGT (Lefranc MP (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid mutations within the heavy chain CDRs that had been previously selected for further analysis in Example 13 were systematically combined two or more at a time (MYT7816-MYT7835). In cases where the starting CDR residue was a histidine, it was mutated to an alanine. Antibody variants with two or more histidine or alanine mutations in the heavy chain CDRs were generated by co-transfection of Expi293 cells with a) one heavy chain combinations sequence variant, and b) the corresponding starting ABPC light chain using methods known to the art. After allowing for four days of protein expression, cell culture supernatants were collected, quantified by SDS-PAGE analysis (FIG. 19), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20) pH 7.4, and the sensor was associated with 50 nM of PTK7 (Recombinant human CCK4/PTK7 Protein (His Tag), Sino Biological 19399-H08H, Lot No. MB14AP1271) in 1×PBST pH 7.4 for 120 sec to generate an association curve. In the dissociation phase, the antibody-antigen complex on the sensor was exposed to 1×PBST pH 7.4 or pH 5.4 for 300 sec. Association and dissociation phase curves were examined for the starting ABPC antibody (with no substitutions) and each corresponding antibody variant at pH 5.4 and pH 7.4 to inform on two criteria: a) enhanced dissociation (i.e., higher koff values) at pH 5.4 due to histidine or alanine substitution compared to the starting ABPC (with no substitutions), and b) reduced dissociation at pH 7.4 (i.e., lower koff values) compared to pH 5.4 in the antibody variant itself and with the starting ABPC (with no substitutions). Heavy chain combinations variants that showed either enhanced dissociation at pH 5.4 or reduced dissociation at pH 7.4 or both (as compared to the starting ABPC), as shown in FIG. 20 were selected for further analysis (e.g., MYT7816, MYT7817, MYT7818, MYT7819, MYT7820, MYT7821, MYT7822, MYT7823, MYT7824, MYT7825, MYT7826, MYT7827, MYT7828, MYT7829, MYT7830, MYT7832, MYT7833, MYT7834, and MYT7835).

### Example 16. Construction and Screening of pH-Engineered PTK7 ABPCs

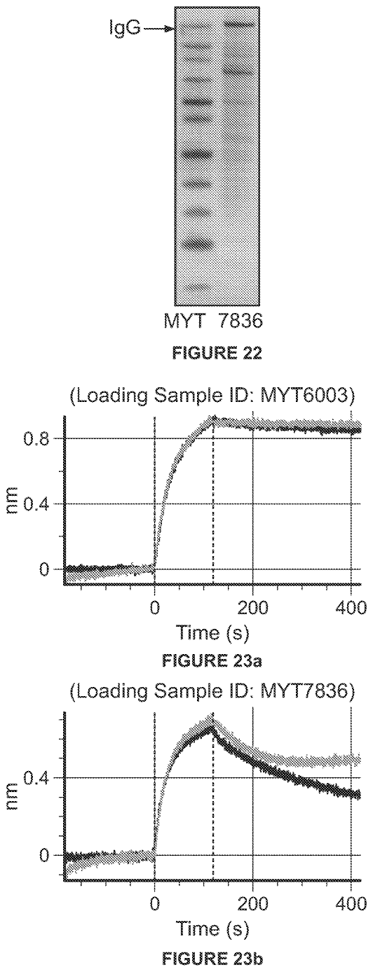

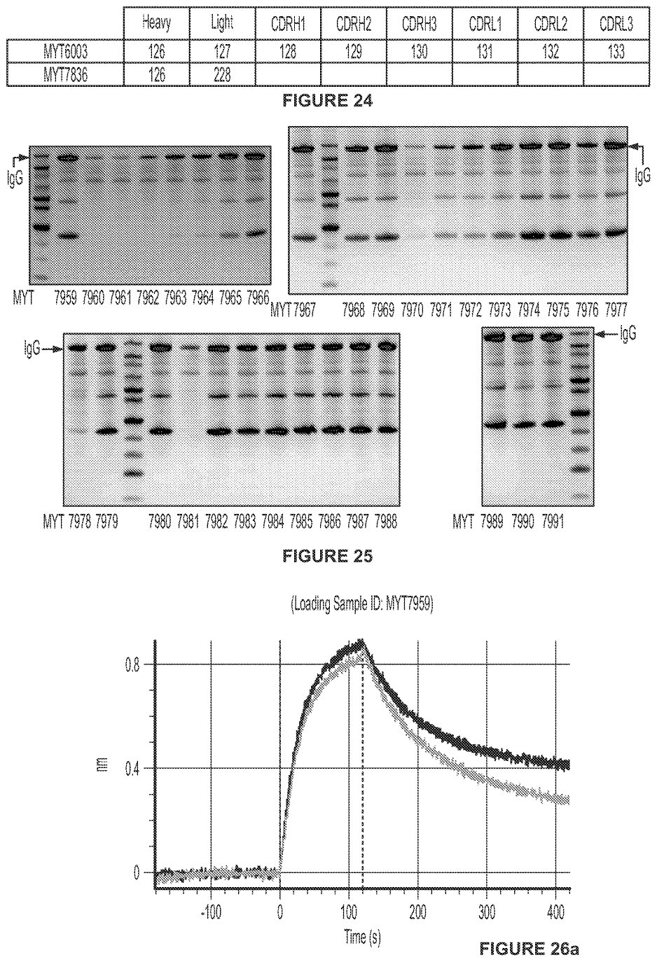

Multiple PTK7-binding monoclonal antibodies have been described in the literature and can be used as a template for engineering pH-dependent binding [Terrett, J et al. “Human Monoclonal Antibodies to Protein Tyrosine Kinase 7 (PTK7)”, U.S. Pat. No. 9,102,738 B2 (2015)]. We selected 7C8 (Heavy chain SEQ ID NO: 126, Light chain SEQ ID NO: 127) as a PTK7-binding monoclonal antibody for pH engineering via histidine scanning. Briefly, CDRs in the light chain were identified using the methods described by Kabat et al (Kabat et al. (1992) Sequences of Proteins of Immunological Interest, DIANE publishing) and IMGT (Lefranc MP (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid mutations within the light chain CDRs that had been previously selected for further analysis in Example 14 were systematically combined two or more at a time (MYT7836). In cases where the starting CDR residue was a histidine, it was mutated to an alanine. Antibody variants with two or more histidine or alanine mutations in the light chain CDRs were generated by co-transfection of Expi293 cells with a) one light chain combinations sequence variant, and b) the corresponding starting ABPC heavy chain using methods known to the art. After allowing for four days of protein expression, cell culture supernatants were collected, quantified by SDS-PAGE analysis (FIG. 22), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM PTK7 (Recombinant human CCK4/PTK7 Protein (His Tag), Sino Biological 19399-H08H, Lot No. MB14AP1271) in 1×PBST, pH 7.4, for 120 sec to generate an association curve. In the dissociation phase, the antibody-antigen complex on the sensor was exposed to 1×PBST, pH 7.4 or pH 5.4 for 300 sec. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Light chain combinations variants that showed either enhanced dissociation at pH 5.4 or reduced dissociation at pH 7.4 or both (as compared to the starting ABPC), as shown in FIG. 23 were selected for further analysis (e.g., MYT7836).

### Example 17. Construction and Screening of pH-Engineered PTK7 ABPCs

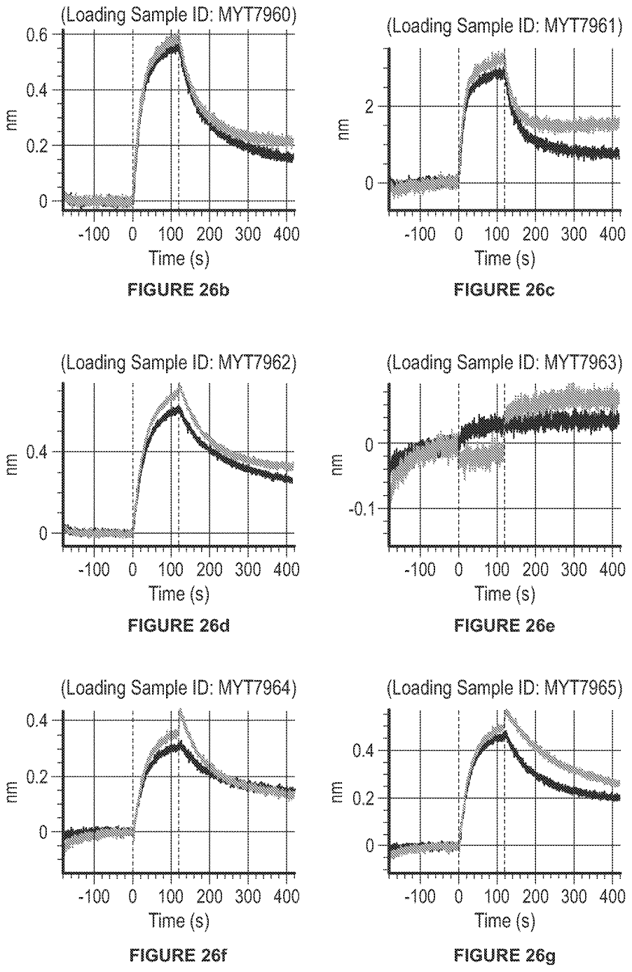

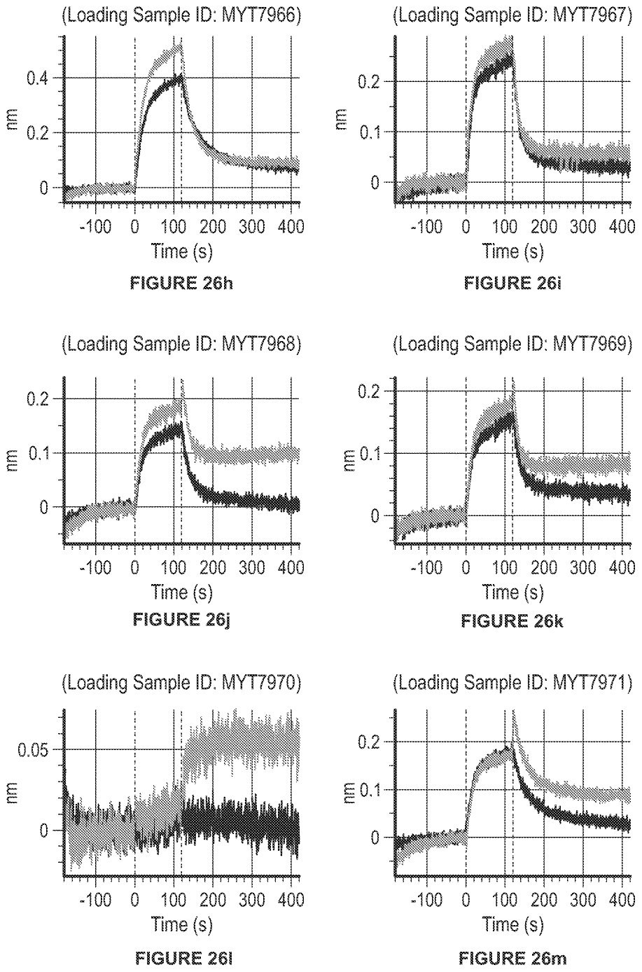

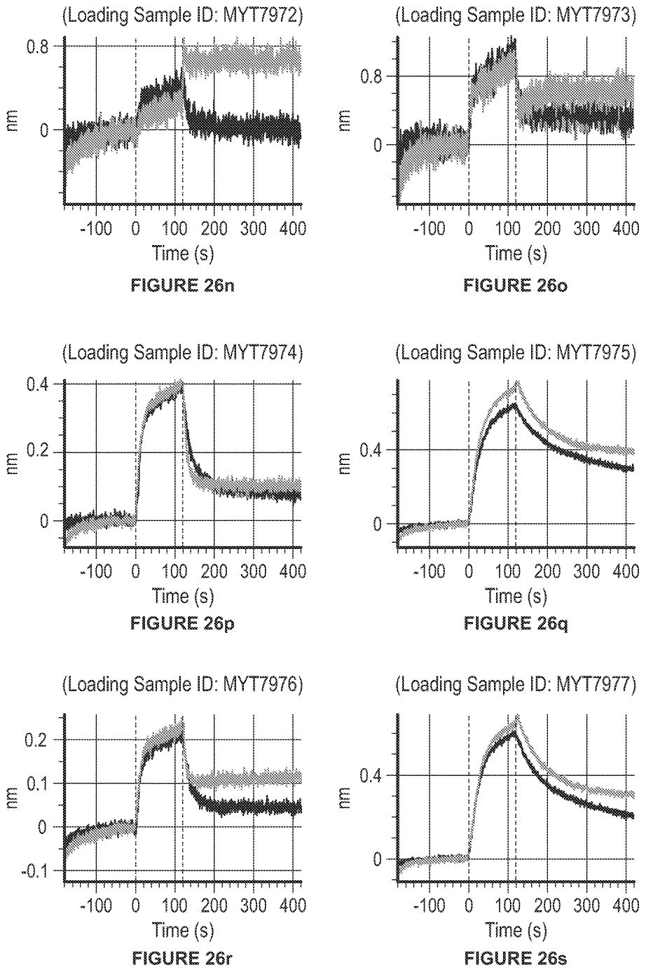

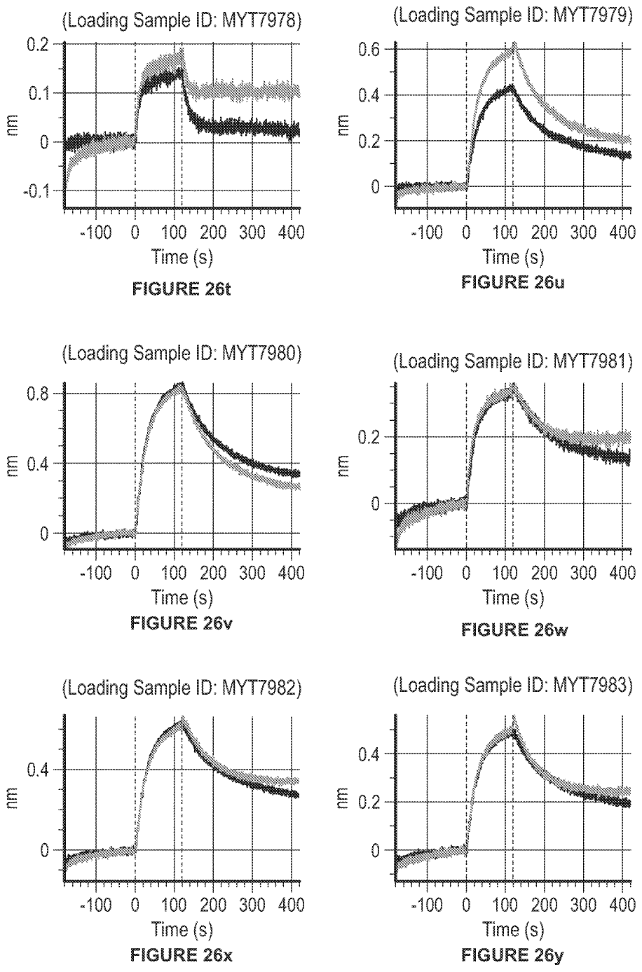

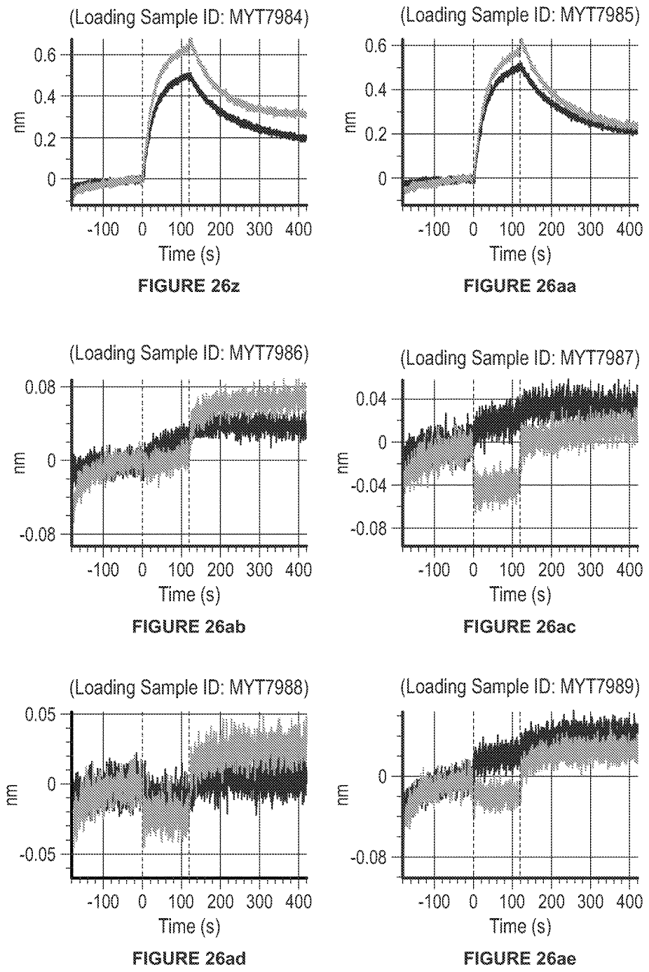

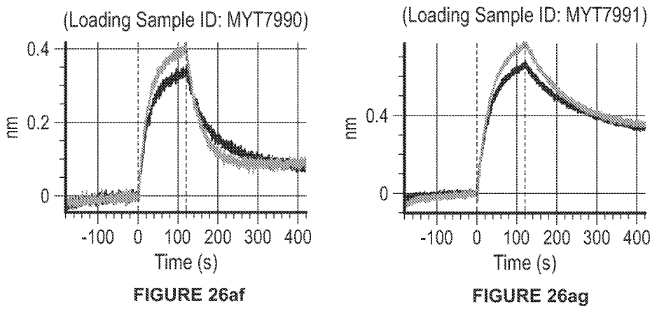

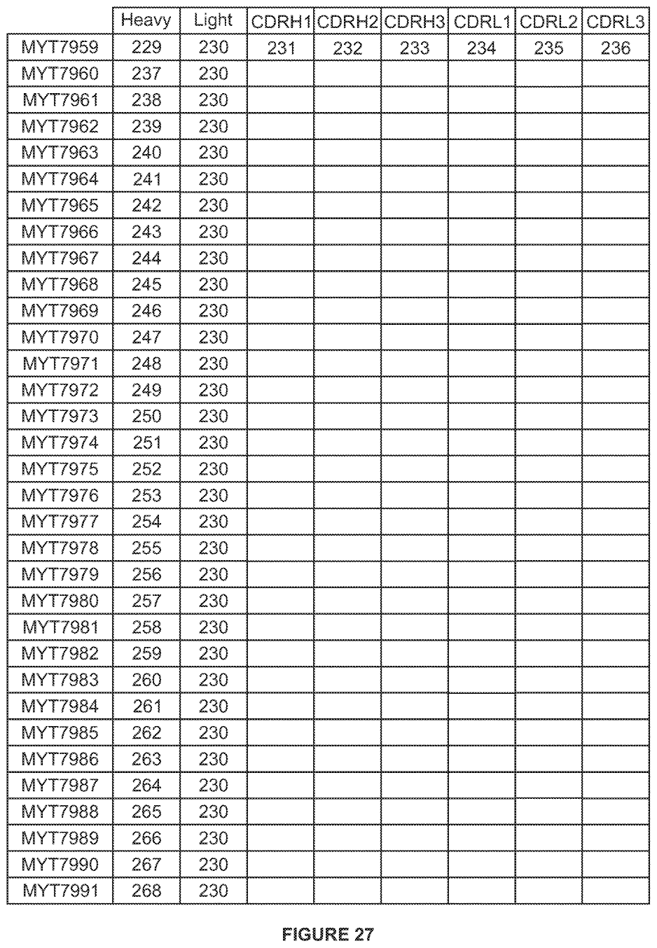

Multiple PTK7-binding monoclonal antibodies have been described in the literature and can be used as a template for engineering pH-dependent binding [Terrett, J et al. “Human Monoclonal Antibodies to Protein Tyrosine Kinase 7 (PTK7)”, U.S. Pat. No. 9,102,738 B2 (2015)]. We selected 12C6 (Heavy chain SEQ ID NO: 229, Light chain SEQ ID NO: 230) as a PTK7-binding monoclonal antibody for pH engineering via histidine scanning. Briefly, CDRs in the heavy chain were identified using the methods described by Kabat et al (Kabat et al. (1992) Sequences of Proteins of Immunological Interest, DIANE publishing) and IMGT (Lefranc MP (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the heavy chain CDRs were systematically substituted with a histidine, one at a time (MYT7960-MYT7991). In cases where the starting CDR residue was a histidine, it was mutated to an alanine. Antibody variants with only one histidine or alanine mutation in a heavy chain CDR were generated by co-transfection of Expi293 cells with a) one heavy chain sequence variant, and b) the corresponding starting ABPC light chain using methods known to the art. After allowing for four days of protein expression, cell culture supernatants were collected, quantified by SDS-PAGE analysis (FIG. 25), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. A baseline was established using 1×PBST (50 mM Potassium Phosphate Buffer+150 mM NaCl+0.05% Tween 20) pH 7.4, and the sensor was associated with 50 nM of PTK7 (Recombinant human CCK4/PTK7 Protein (His Tag), Sino Biological 19399-H08H, Lot No. MB14AP1271) in 1×PBST pH 7.4 for 120 sec to generate an association curve. In the dissociation phase, the antibody-antigen complex on the sensor was exposed to 1×PBST pH 7.4 or pH 5.4 for 300 sec. Association and dissociation phase curves were examined for the starting ABPC antibody (with no substitutions) and each corresponding antibody variant at pH 5.4 and pH 7.4 to inform on two criteria: a) enhanced dissociation (i.e., higher koff values) at pH 5.4 due to histidine or alanine substitution compared to the starting ABPC, (with no substitutions), and b) reduced dissociation at pH 7.4 (i.e., lower koff values) compared to pH 5.4 in the antibody variant itself and with the starting ABPC (with no substitutions). Heavy chain variants that showed either enhanced dissociation at pH 5.4 or reduced dissociation at pH 7.4 or both (as compared to starting ABPC) as shown in FIG. 26 were selected for further analysis (e.g., MYT7961, MYT7966, MYT7967, MYT7968, MYT7969, MYT7971, MYT7974, MYT7976, MYT7978, and MYT7990). It was also noted that while some histidine and alanine mutations obliterated PTK7 binding (e.g., MYT7963, MYT7970, MYT7972, MYT7973, MYT7986, MYT7987, MYT7988, and MYT7989), some histidine and alanine mutations were tolerated with little (e.g., less than 1-fold change in dissociation constant KD or dissociation rate) or no change in PTK7 binding kinetics (e.g., MYT7962, MYT7964, MYT7965, MYT7975, MYT7977, MYT7979, MYT7980, MYT7981, MYT7982, MYT7983, MYT7984, MYT7985, and MYT7991).

Especially because histidine is a large, positively charged amino acid, these variants with no change were noted as positions in the heavy chain that may tolerate a wide range of mutations and lead to antibodies with different sequence but similar binding properties, a designation that is not otherwise apparent.

### Example 18. Construction and Screening of pH-Engineered PTK7 ABPCs

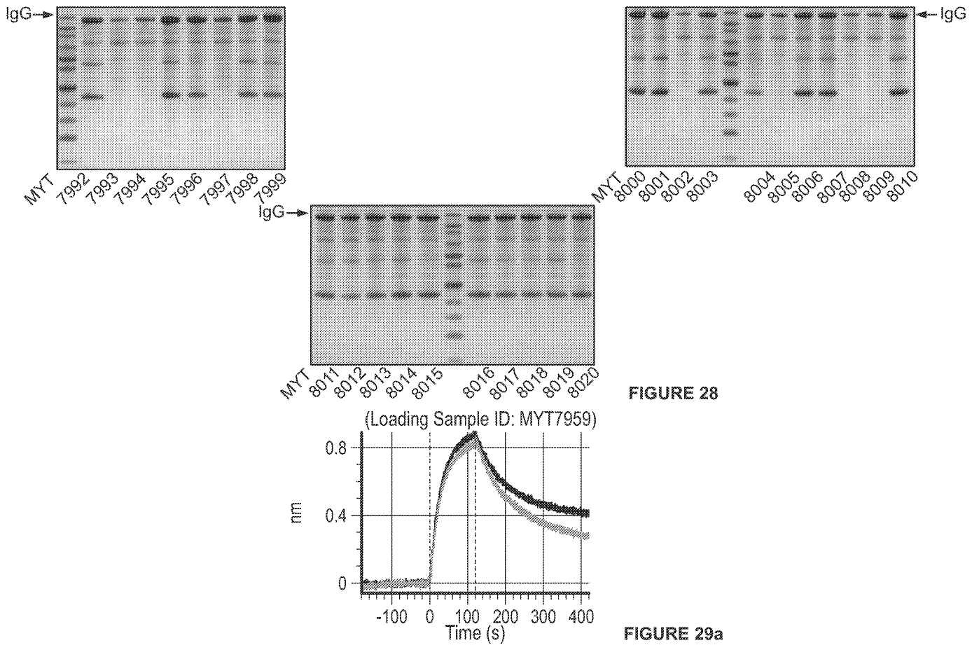

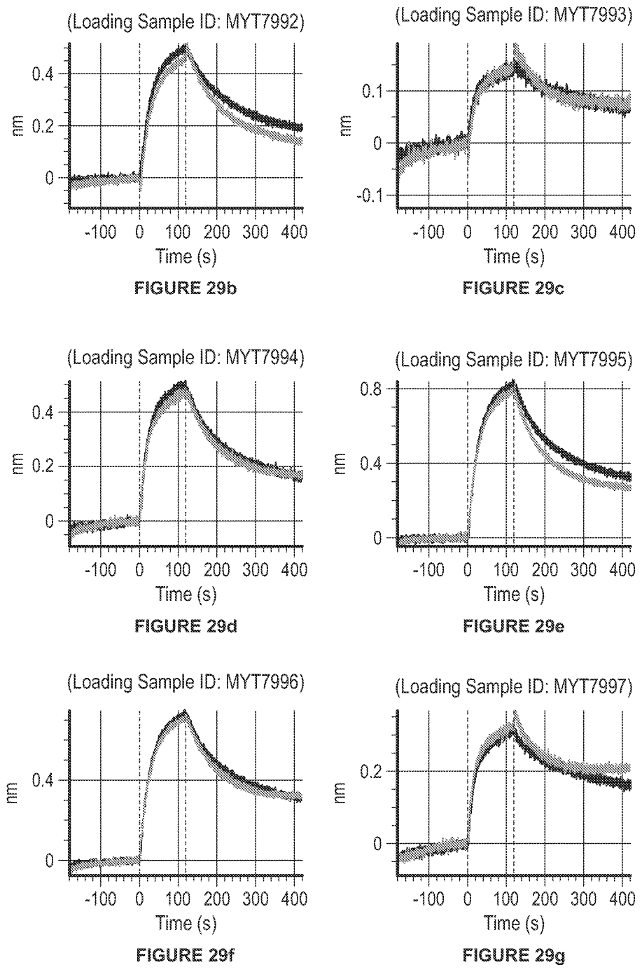

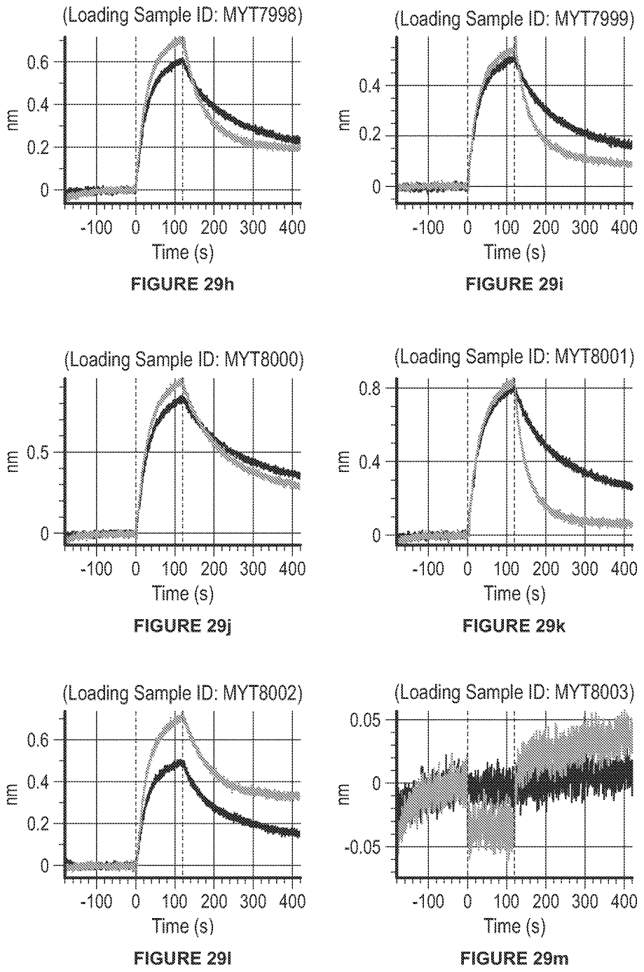

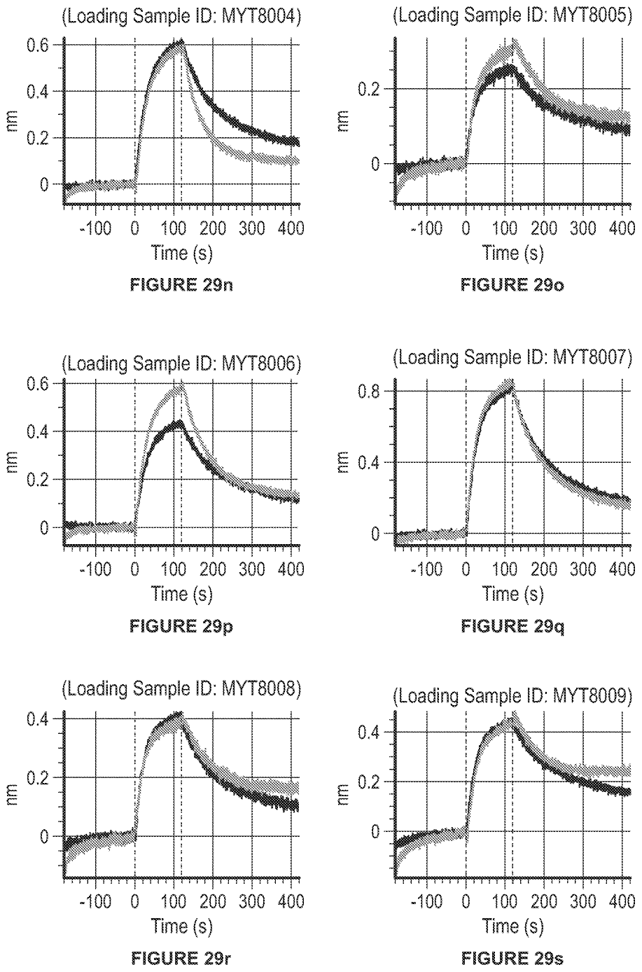

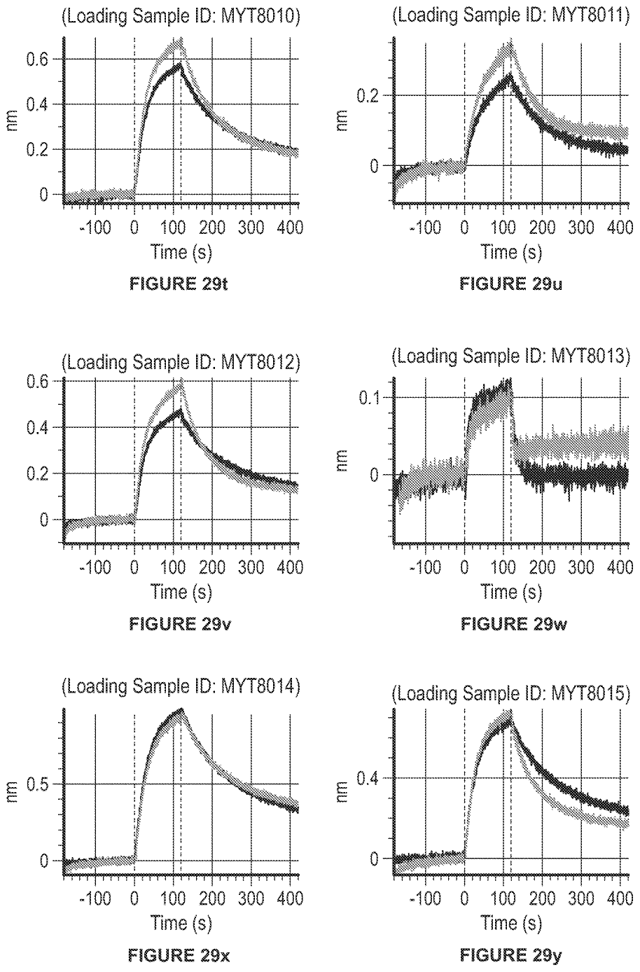

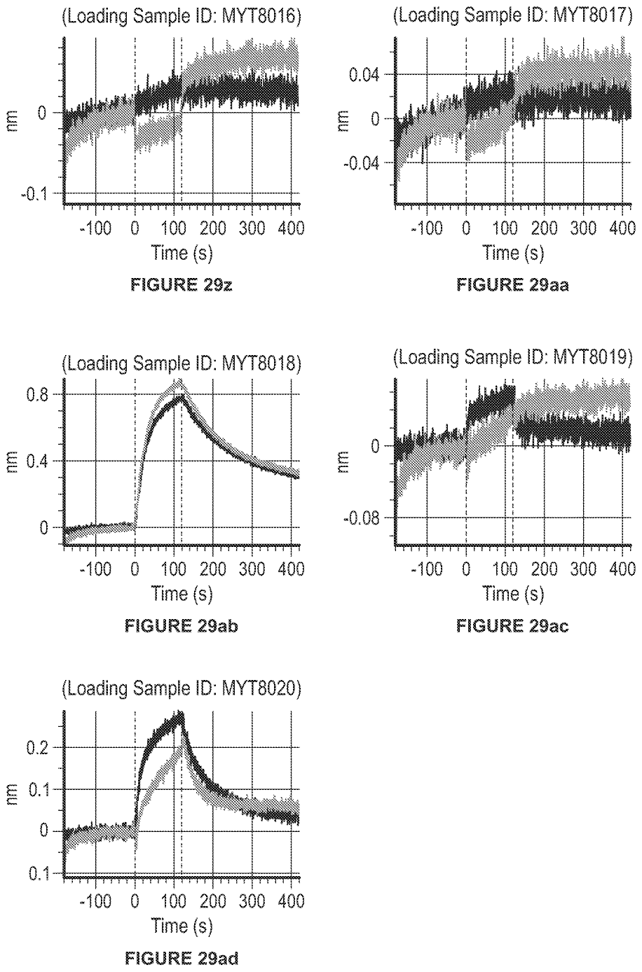

Multiple PTK7-binding monoclonal antibodies have been described in the literature and can be used as a template for engineering pH-dependent binding [Terrett, J et al. “Human Monoclonal Antibodies to Protein Tyrosine Kinase 7 (PTK7)”, U.S. Pat. No. 9,102,738 B2 (2015)]. We selected 12C6 (Heavy chain SEQ ID NO: 229, Light chain SEQ ID NO: 230) as a PTK7-binding monoclonal antibody for pH engineering via histidine scanning. Briefly, CDRs in the light chain were identified using the methods described by Kabat et al (Kabat et al. (1992) Sequences of Proteins of Immunological Interest, DIANE publishing) and IMGT (Lefranc MP (1999) “The IMGT unique numbering for Immunoglobulins, T cell receptors and Ig-like domains” The Immunologist 7, 132-136), and for each CDR, residues falling under cither or both Kabat and IMGT CDR definitions were called as CDR residues. To generate pH-dependent sequence variants, individual amino acid residues within the light chain CDRs were systematically substituted with a histidine, one at a time (MYT7992-MYT8020). In cases where the starting CDR residue was a histidine, it was mutated to an alanine. Antibody variants with only one histidine or alanine mutation in a light chain CDR were generated by co-transfection of Expi293 cells with a) one light chain sequence variant, and b) the corresponding starting ABPC heavy chain using methods known to the art. After allowing for four days of protein expression, cell culture supernatants were collected, quantified by SDS-PAGE analysis (FIG. 28), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20) pH 7.4, and the sensor was associated with 50 nM of PTK7 (Recombinant human CCK4/PTK7 Protein (His Tag), Sino Biological 19399-H08H, Lot No. MB14AP1271) in 1×PBST pH 7.4 for 120 sec to generate an association curve. In the dissociation phase, the antibody-antigen complex on the sensor was exposed to 1×PBST pH 7.4 or pH 5.4 for 300 sec. Association and dissociation phase curves were examined for the starting ABPC antibody (with no substitutions) and each corresponding antibody variant at pH 5.4 and pH 7.4 to inform on two criteria: a) enhanced dissociation (i.e., higher koff values) at pH 5.4 due to histidine or alanine substitution compared to the starting ABPC (with no substitutions), and b) reduced dissociation at pH 7.4 (i.e., lower koff values) compared to pH 5.4 in the antibody variant itself and with the starting ABPC (with no substitutions). Light chain variants that showed either enhanced dissociation at pH 5.4 or reduced dissociation at pH 7.4 or both (as compared to the starting ABPC), as shown in FIG. 29 were selected for further analysis (e.g., MYT7998, MYT7999, MYT8001, MYT8004, MYT8006, MYT8010, MYT8012, MYT8013, MYT8015, and MYT8020). It was also noted that while some histidine and alanine mutations obliterated binding to PTK7 (e.g., MYT8003, MYT8016, MYT8017, and MYT8019), other mutations were tolerated with little (e.g., less than 1-fold change in dissociation constant KD or dissociation rate) or no change in PTK7 binding kinetics (e.g., MYT7992, MYT7993, MYT7995, MYT7996, MYT8000, MYT8002, MYT8005, MYT8009, and MYT8018).

Especially because histidine is a large, positively charged amino acid, these variants with no change were noted as positions in the light chain that may tolerate a wide range of mutations and lead to antibodies with different sequence but similar binding properties, a designation that is not otherwise apparent.

### Example 19. Characterization of Cellular Internalization and Endolysosomal Delivery of pH Engineered Anti-PTK7 ABPCs

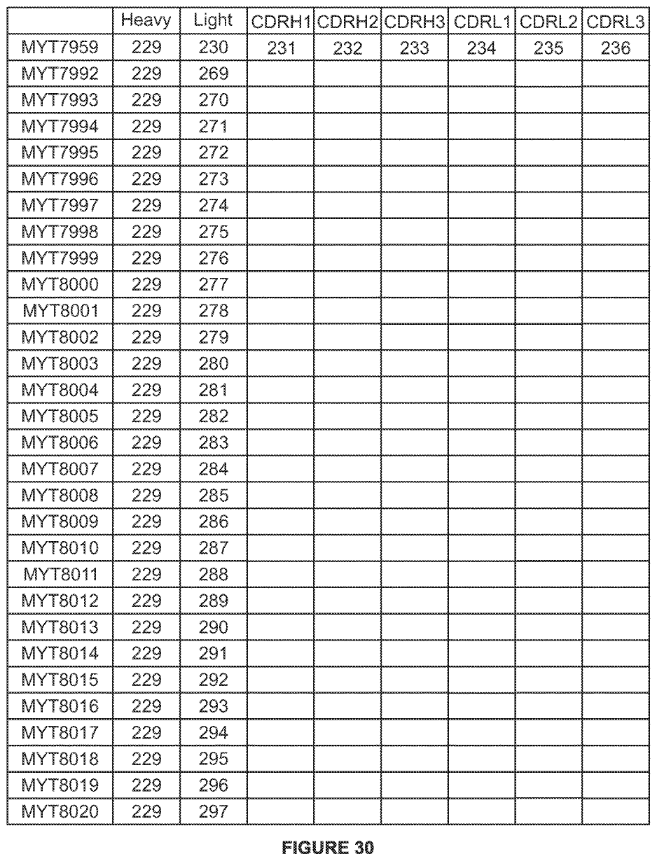

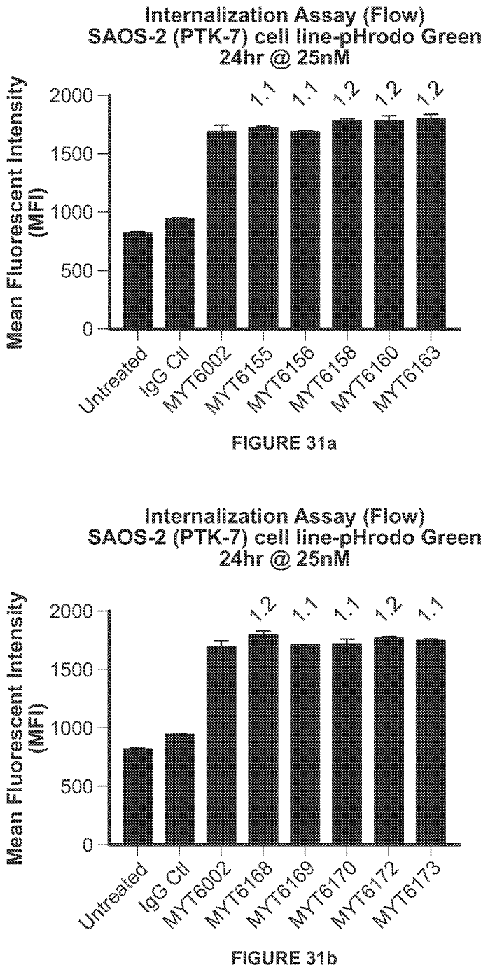

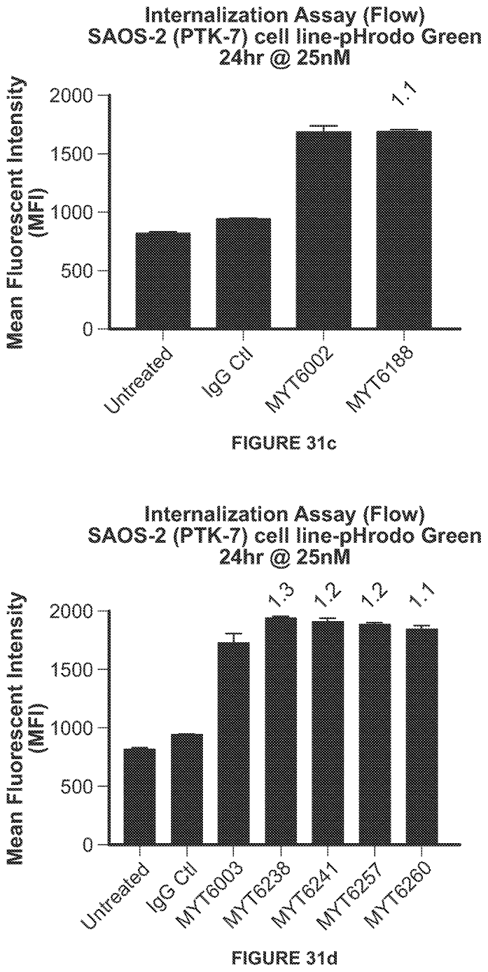

Selected anti-PTK7 pH engineered antibody variants from Examples 9 and 13 were analyzed for internalization and endolysosomal delivery in Saos-2 cells (PTK7+). Saos-2 cells (ATCC HTB-85) were collected and resuspended in culture medium (McCoy's 5a Medium (ATCC 30-2007) plus 15% GenClone heat inactivated fetal bovine serum (HI FBS) (Genesee Scientific; 25-514H)). Cell counts were determined using trypan blue staining and the Countess II FL Automated Cell Counter (ThermoFisher, AMQAF1000). Cells were then diluted to 75,000 cells/mL and 100 μl/well was seeded into 96-well flat bottom cell culture plates (Genesee Scientific; 25-109) for overnight incubation at 37° C. and 5% CO². The following day, anti-PTK7 pH engineered antibody variants, starting ABPC antibodies, control IgG1 isotype control (BP0297, Bioxcell), and vehicle control were diluted in native culture media, and then mixed 1:1 with a 3× molar ratio Zenon pHrodo iFL Human IgG Labeling Reagent (ThermoFisher; Z25611). The mixture was incubated for 30 minutes at room temperature, the existing media was removed from the plated cells and the mixture was added. The mixture of cells, anti-PTK7 antibody variants, and Zenon pHrodo iFL Human IgG Labeling Reagent was incubated at 37° C., 5% CO₂ for 24 hours. Following incubation, the media was removed and cells were washed with 100 μL of room temperature culture medium. Following the wash 50 μL of Trypsin solution (ThermoFisher Scientific; 12605036) was added and the plate incubated at 37° C. until most of the cells were detached, followed by addition of 50 μL of culture medium. Cells were transferred to a 96-well U-bottom plate (Genesee Scientific; 25-221) and spun down at 2000 RPM for 2 minutes. Cells were washed 2× with 200 μL of ice-cold FACS buffer (phosphate buffered saline (PBS), pH 7.4+2 mM ethylenediaminetetraacetic acid (EDTA)+2% (v/v) HI FBS) before being resuspended in 150 μL of FACS buffer. Cells (Median green fluorescence intensity was detected using an Attune NxT flow cytometer (ThermoFisher Scientific). Data was analyzed using Flowjo analysis software. pHrodo green is a pH sensitive dye that fluoresces in the low pH environment of the endosomes and lysosomes and therefore can be used to quantify antibody internalization and endolysosomal delivery. Internalization and endolysosomal delivery of anti-PTK7 starting ABPC's and variants at 25 nM in Saos-2 (PTK7+) cells is shown in FIG. 31 as measured by pHrodo green median fluorescence intensity. Several pH engineered anti-PTK7 antibody variants showed increased median fluorescence intensity relative to their corresponding starting ABPC antibodies demonstrating that increased dissociation at lower pH leads to enhanced internalization and endolysosomal delivery inside cells as shown by increased fluorescence or increased fluorescence as compared to IgG1 isotype control. Increased endolysosomal delivery is quantitated for each pH engineered anti-PTK7 antibody variant on the top of each bar as a ratio of: the variant's median fluorescence intensity minus the median fluorescence intensity of the IgG control, then all divided by the variant's corresponding starting ABPC's median fluorescence intensity minus the median fluorescence intensity of the IgG control. For example MYT6155, MYT6156, MYT6158, MYT6160, MYT6163, MYT6168, MYT619, MYT6170, MYT6172, MYT6173, and MYT6188, antibody variants of cofetuzumab, showed increased internalization and endolysosomal delivery relative to cofetuzumab (MYT6002). For example MYT6238, MYT6241, MYT6257, and MYT6260, antibody variants of 7C8, showed increased internalization and endolysosomal delivery relative to 7C8 (MYT6003).

Such pH engineered anti-MET antibody variants with increased median fluorescence intensity relative to their starting ABPC antibodies were selected for further analysis

### Example 20. Characterization of Cellular Internalization and Endolysosomal Delivery of pH Engineered Anti-PTK7 ABPCs

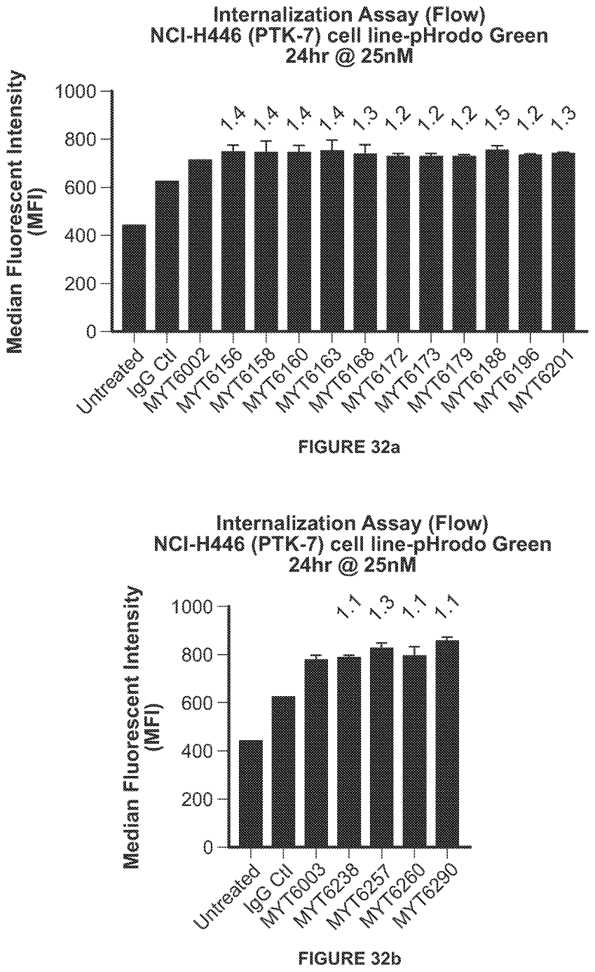

Selected anti-PTK7 pH engineered antibody variants from Examples 9, 10, 13, and 14 were analyzed for internalization and endolysosomal delivery in NCI-H446 cells (PTK7+). NCI-H446 cells (ATCC HTB-171) were collected and resuspended in culture medium (RPMI-1640 medium (ATCC 30-2001) plus 10% GenClone heat inactivated fetal bovine serum (HI FBS) (Genesee Scientific; 25-514H)). Cell counts were determined using trypan blue staining and the Countess II FL Automated Cell Counter (ThermoFisher; AMQAF1000). Cells were then diluted to 200,000 cells/mL and 50 μl/well was seeded into 96-well flat bottom cell culture plates (Genesee Scientific; 25-109) for overnight incubation at 37° C. and 5% CO². The following day, anti-PTK7 pH engineered antibody variants, starting ABPC antibodies, control IgG1 isotype control (BP0297, Bioxcell), and vehicle control were diluted in native culture media, and then mixed 1:1 with a 3× molar ratio Zenon pHrodo iFL Human IgG Labeling Reagent (ThermoFisher; Z25611). The mixture was incubated for 30 minutes at room temperature, the mixture was added 1:1 to the existing plated cells. The mixture of cells, anti-PTK7 antibody variants, and Zenon pHrodo iFL Human IgG Labeling Reagent was incubated at 37° C., 5% CO₂ for 24 hours. Following incubation, the media was removed and cells were washed with 100 μL of room temperature culture medium. Following the wash 50 μL of Trypsin solution (ThermoFisher Scientific; 12605036) was added and the plate incubated at 37° C. until most of the cells were detached, followed by addition of 50 μL of culture medium. Cells were transferred to a 96-well U-bottom plate (Genesee Scientific; 25-221) and spun down at 2000 RPM for 2 minutes. Cells were washed 2× with 200 μL of ice cold FACS buffer (phosphate buffered saline (PBS), pH 7.4+2 mM ethylenediaminetetraacetic acid (EDTA)+2% (v/v) HI FBS) before being resuspended in 150 μL of FACS buffer. Cells (Median green fluorescence intensity was detected using an Attune NxT flow cytometer (ThermoFisher Scientific). Data was analyzed using Flowjo analysis software. pHrodo green is a pH sensitive dye that fluoresces in the low pH environment of the endosomes and lysosomes and therefore can be used to quantify antibody internalization and endolysosomal delivery. Internalization and endolysosomal delivery of anti-PTK7 starting ABPC's and variants at 25 nM in NCI-H446 (PTK7+) cells is shown in FIG. 32 as measured by pHrodo green median fluorescence intensity. Several pH engineered anti-PTK7 antibody variants showed increased median fluorescence intensity relative to their corresponding starting ABPC antibodies demonstrating that increased dissociation at lower pH leads to enhanced internalization and endolysosomal delivery inside cells as shown by increased fluorescence or increased fluorescence as compared to IgG1 isotype control. Increased endolysosomal delivery is quantitated for each pH engineered anti-PTK7 antibody variant on the top of each bar as a ratio of: the variant's median fluorescence intensity minus the median fluorescence intensity of the IgG control, then all divided by the variant's corresponding starting ABPC's median fluorescence intensity minus the median fluorescence intensity of the IgG control. For example MYT6156, MYT6158, MYT6160, MYT6163, MYT6168, MYT6172, MYT6173, MYT6179, MYT6188, MYT6196, and MYT6201, antibody variants of cofetuzumab, showed increased internalization and endolysosomal delivery relative to cofetuzumab (MYT6002). For example MYT6238, MYT6257, MYT6260, and MYT6290, antibody variants of 7C8, showed increased internalization and endolysosomal delivery relative to 7C8 (MYT6003).

Such pH engineered anti-MET antibody variants with increased median fluorescence intensity relative to their starting ABPC antibodies were selected for further analysis

### Example 21. Characterization of Cellular Internalization and Endolysosomal Delivery of pH Engineered Anti-PTK7 ABPCs

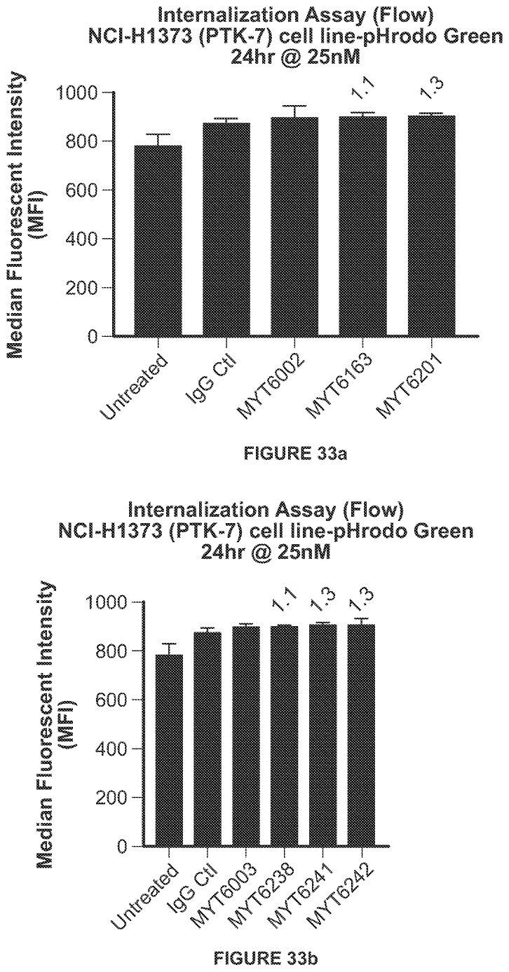

Selected anti-PTK7 pH engineered antibody variants from Examples 9, 10, and 13 were analyzed for internalization and endolysosomal delivery in NCI-H1373 (PTK7+). NCI-H1373 (ATCC CRL-5866) were collected and resuspended in culture medium (RPMI-1640 medium (ATCC 30-2001) plus 10% GenClone heat inactivated fetal bovine serum (HI FBS) (Genesee Scientific; 25-514H)). Cell counts were determined using trypan blue staining and the Countess II FL Automated Cell Counter (ThermoFisher; AMQAF1000). Cells were then diluted to 100,000 cells/mL and 100 μl/well was seeded into 96-well flat bottom cell culture plates (Genesee Scientific; 25-109) for overnight incubation at 37° C. and 5% CO². The following day, anti-PTK.7 pH engineered antibody variants, starting ABPC antibodies, control IgG1 isotype control (BP0297, Bioxcell), and vehicle control were diluted in native culture media, and then mixed 1:1 with a 3× molar ratio Zenon pHrodo iFL Human IgG Labeling Reagent (ThermoFisher; Z25611). The mixture was incubated for 30 minutes at room temperature, the existing media was removed from the plated cells and the mixture was added. The mixture of cells, anti-PTK7 antibody variants, and Zenon pHrodo iFL Human IgG Labeling Reagent was incubated at 37° C., 5% CO2 for 24 hours. Following incubation, the media was removed and cells were washed with 100 μL of room temperature culture medium. Following the wash 50 μL of Trypsin solution (Brand, Catalog) was added and the plate incubated at 37° C. until most of the cells were detached, followed by addition of 50 μL of culture medium. Cells were transferred to a 96-well U-bottom plate (Genesee Scientific; 25-221) and spun down at 2000 RPM for 2 minutes. Cells were washed 2× with 200 μL of ice cold FACS buffer (phosphate buffered saline (PBS), pH 7.4+2 mM ethylenediaminetetraacetic acid (EDTA)+2% (v/v) HI FBS) before being resuspended in 150 μL of FACS buffer. Cells (median green fluorescence intensity was detected using an Attune NxT flow cytometer (ThermoFisher Scientific). Data was analyzed using Flowjo analysis software. pHrodo green is a pH sensitive dye that fluoresces in the low pH environment of the endosomes and lysosomes and therefore can be used to quantify antibody internalization and endolysosomal delivery. Internalization and endolysosomal delivery of anti-PTK7 starting ABPC's and variants at 25 nM in NCI-H1373 (PTK7+) cells is shown in FIG. 33 as measured by pHrodo green median fluorescence intensity. Several pH engineered anti-PTK7 antibody variants showed increased median fluorescence intensity relative to their corresponding starting ABPC antibodies demonstrating that increased dissociation at lower pH leads to enhanced internalization and endolysosomal delivery inside cells as shown by increased fluorescence or increased fluorescence as compared to IgG1 isotype control. Increased endolysosomal delivery is quantitated for each pH engineered anti-PTK7 antibody variant on the top of each bar as a ratio of: the variant's median fluorescence intensity minus the median fluorescence intensity of the IgG control, then all divided by the variant's corresponding starting ABPC's median fluorescence intensity minus the median fluorescence intensity of the IgG control. For example MYT6163 and MYT6201, antibody variants of cofetuzumab, showed increased internalization and endolysosomal delivery relative to cofetuzumab (MYT6002). For example MYT6238, MYT6241, and MYT6242, antibody variants of 7C8, showed increased internalization and endolysosomal delivery relative to 7C8 (MYT6003).

### Example 22. Construction and Screening of pH-Engineered PTK7 ABPCs

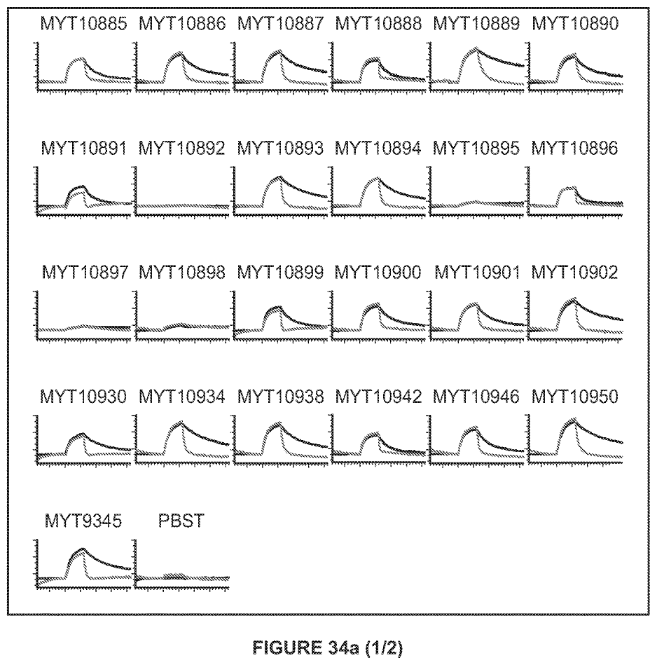

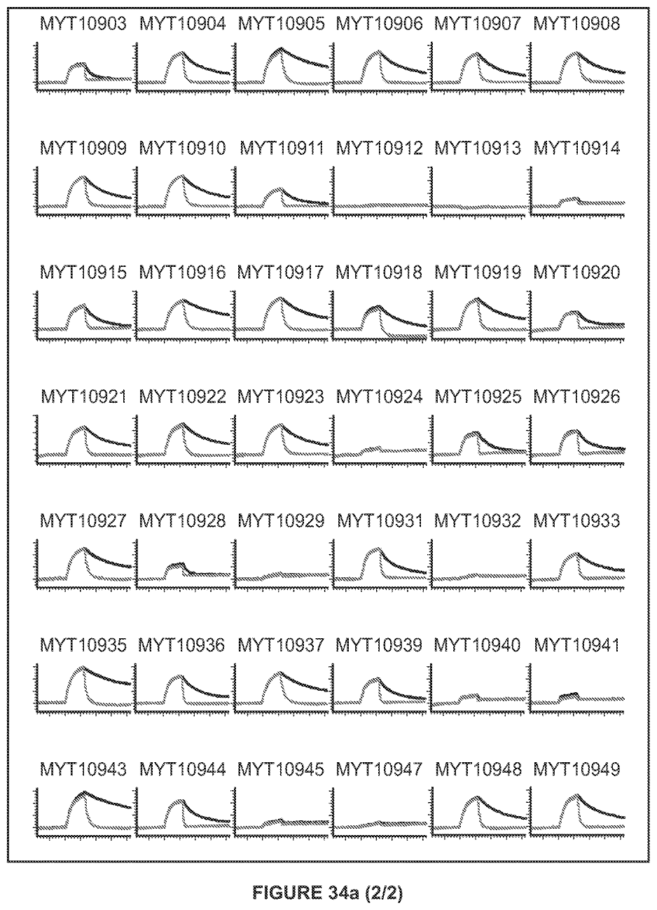

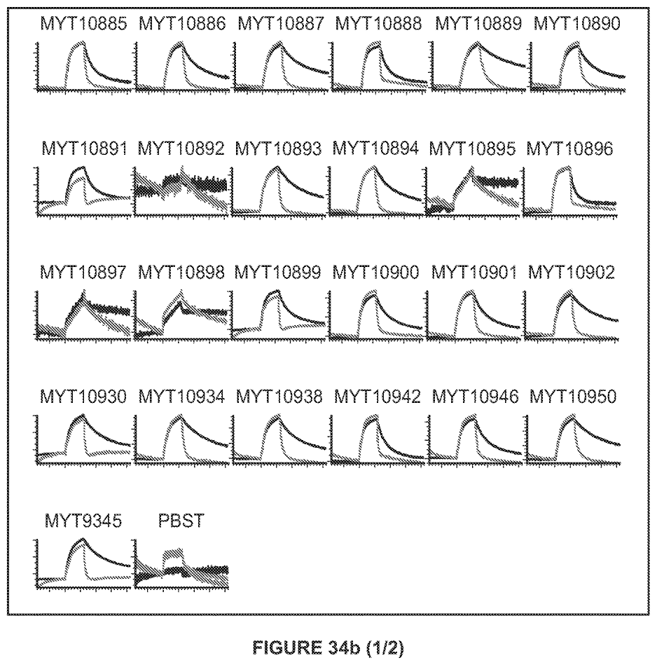

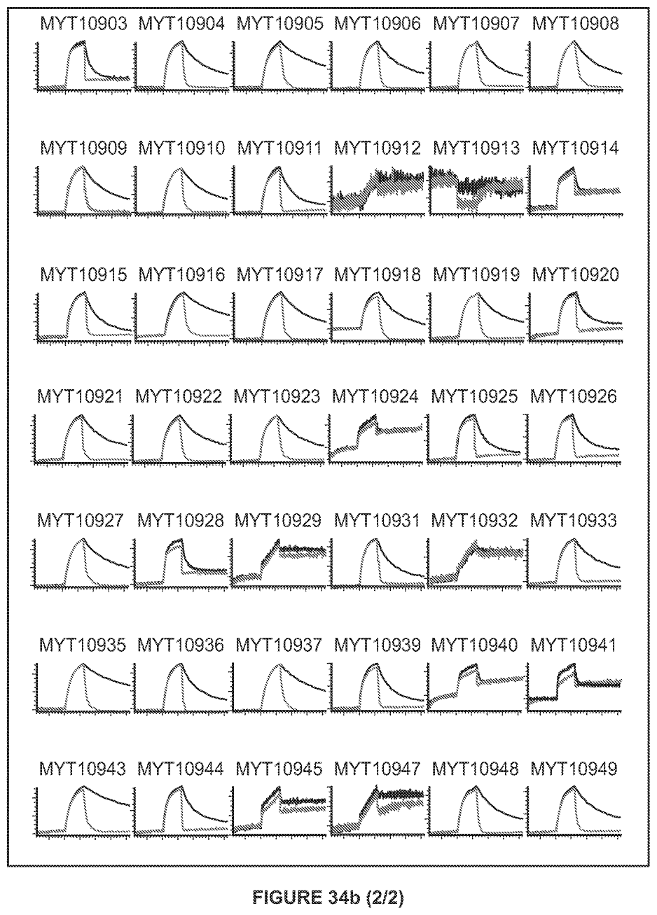

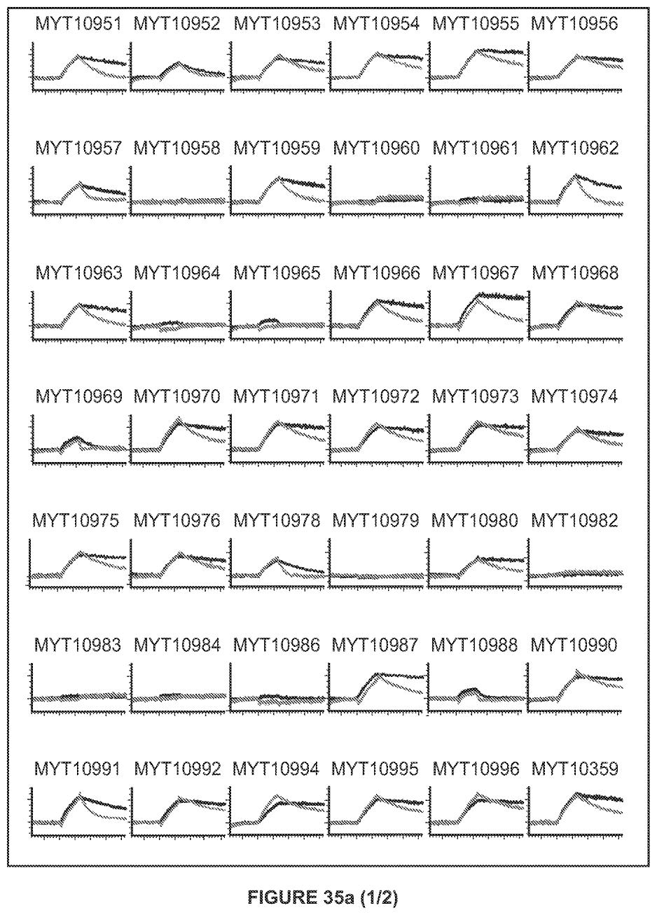

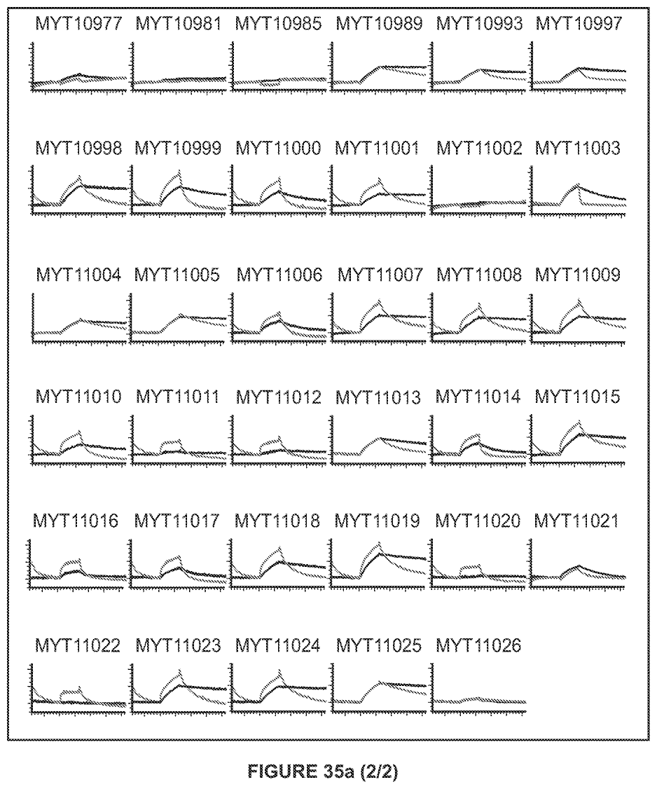

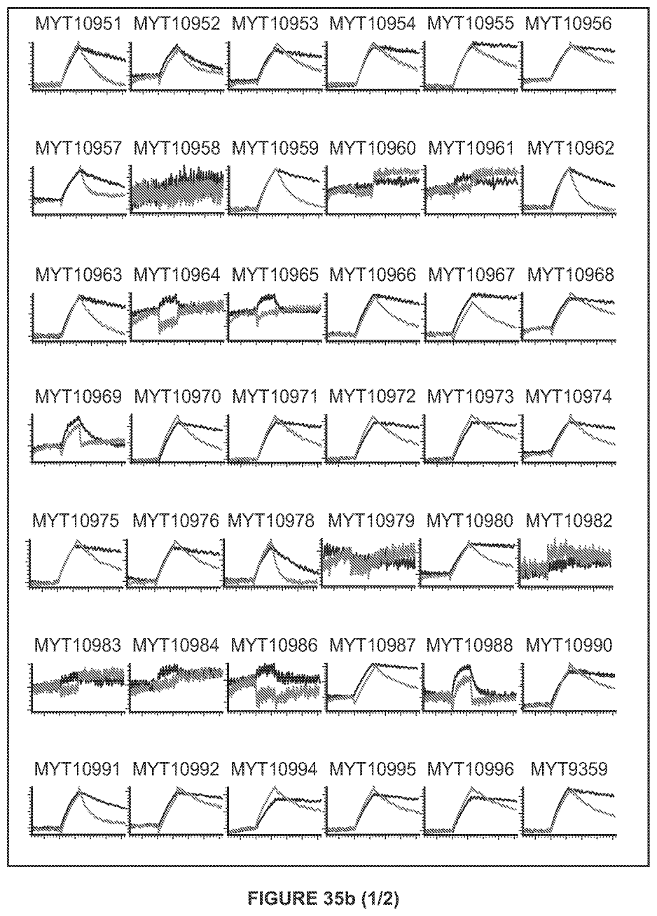

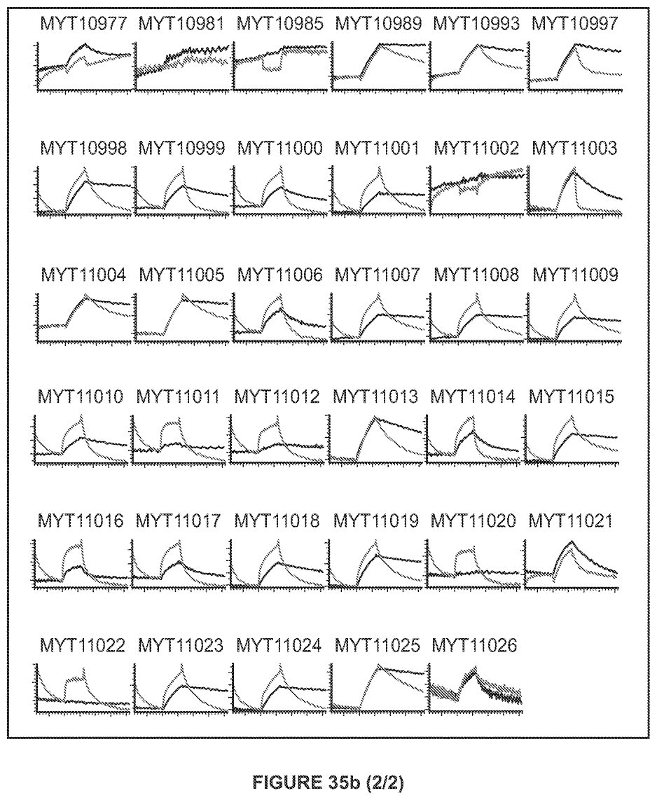

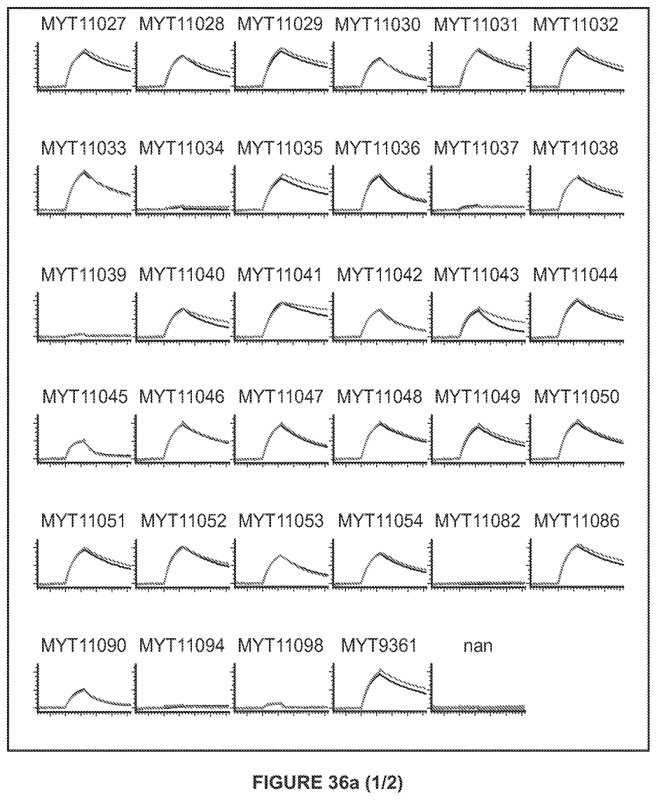

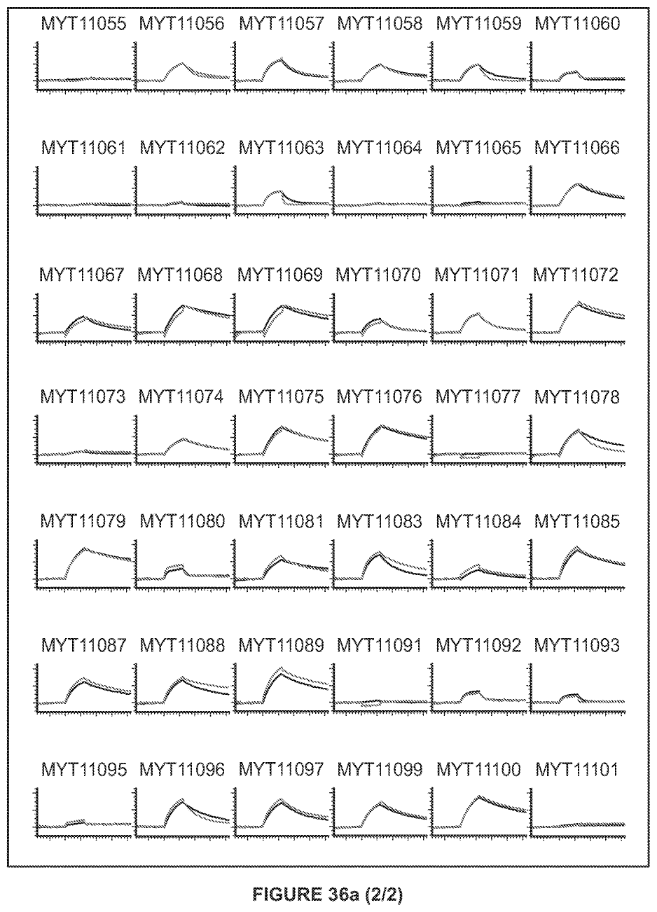

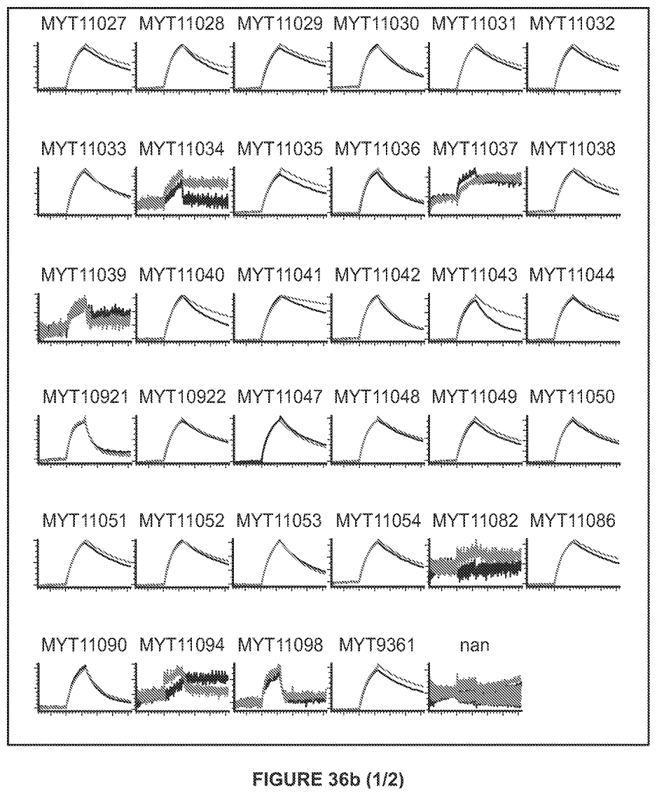

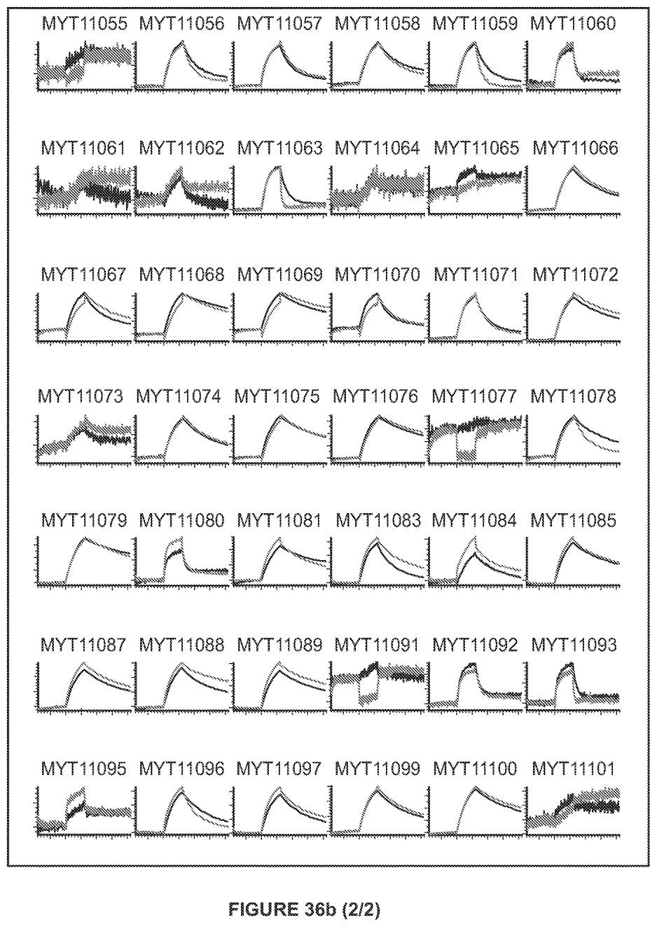

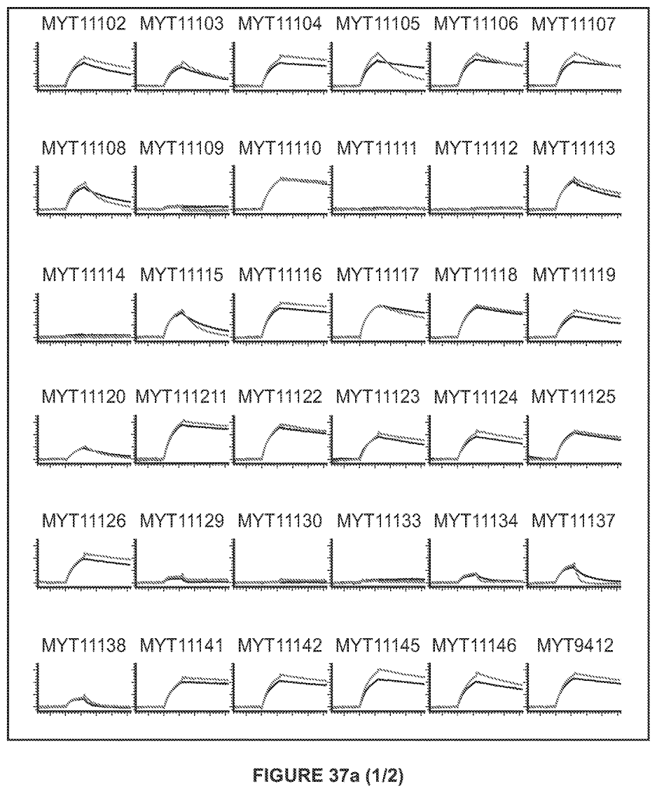

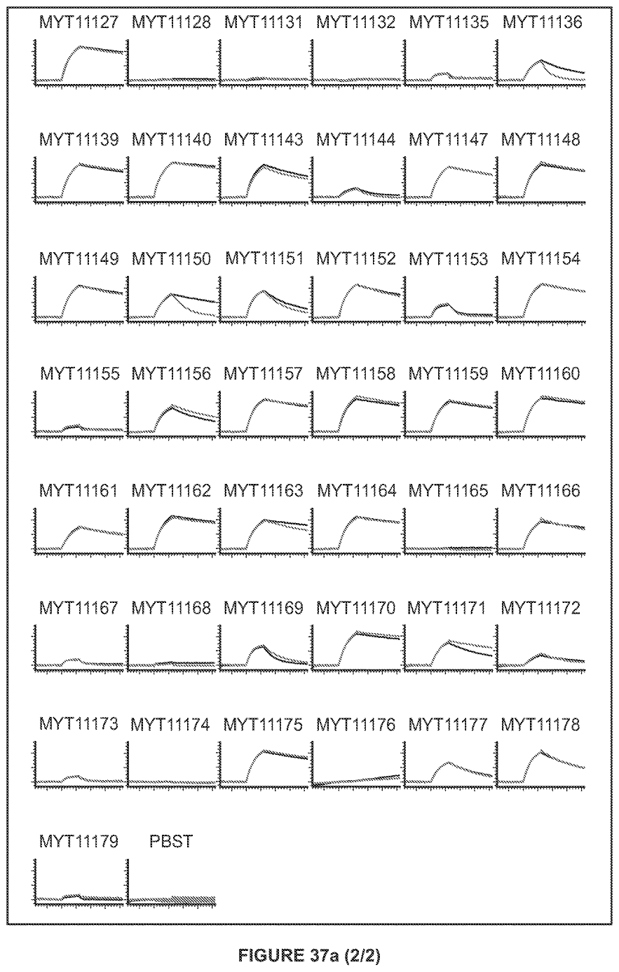

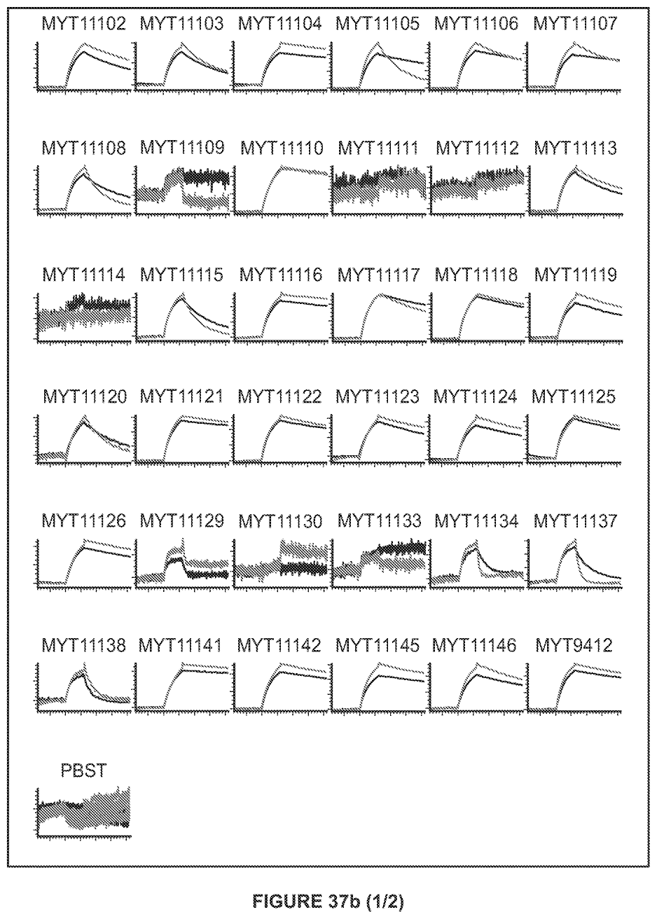

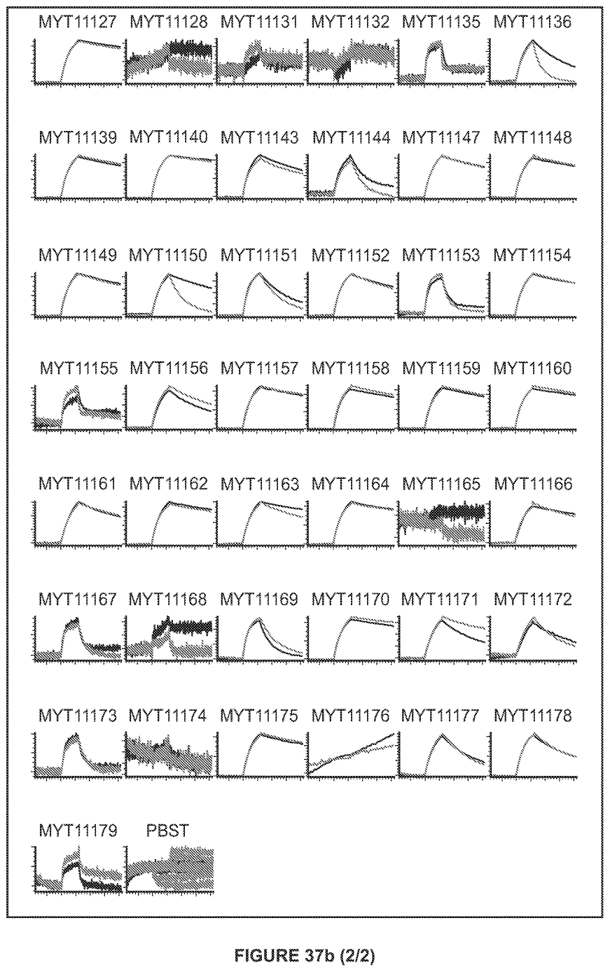

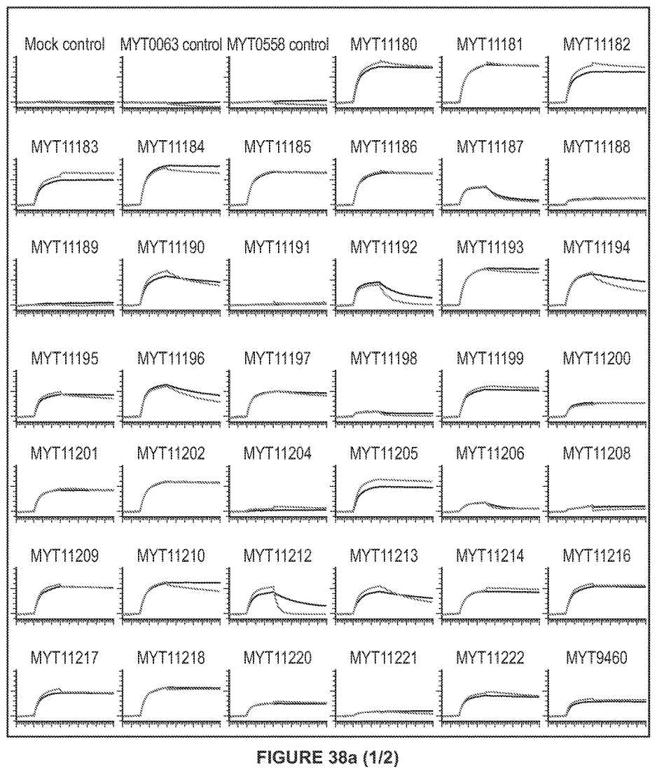

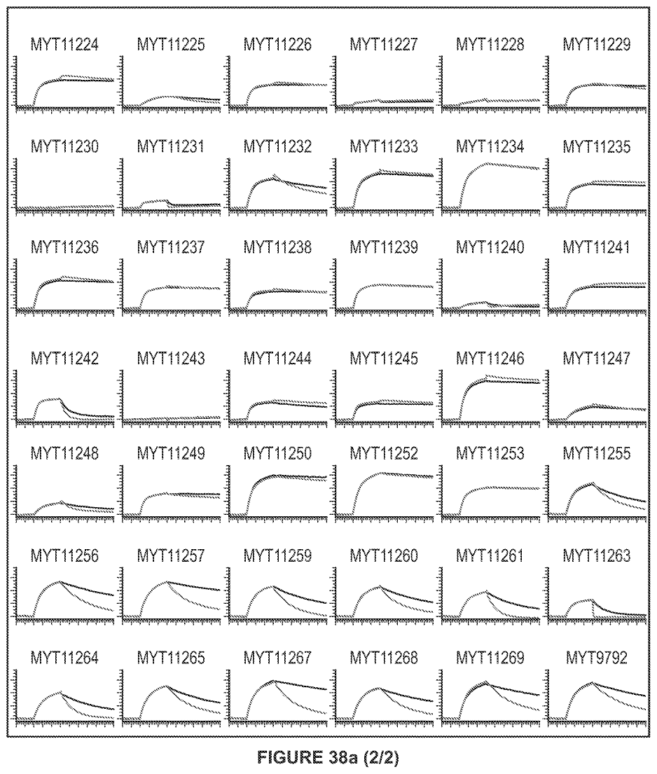

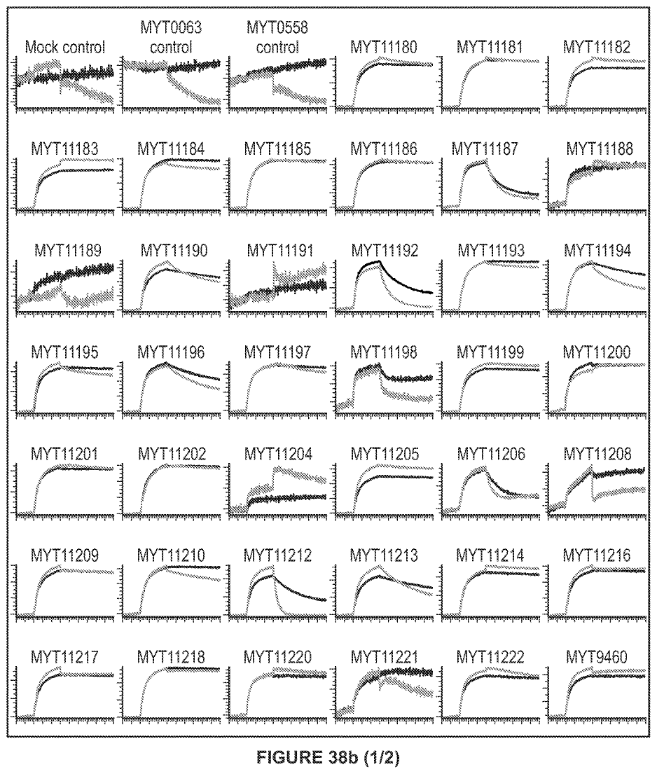

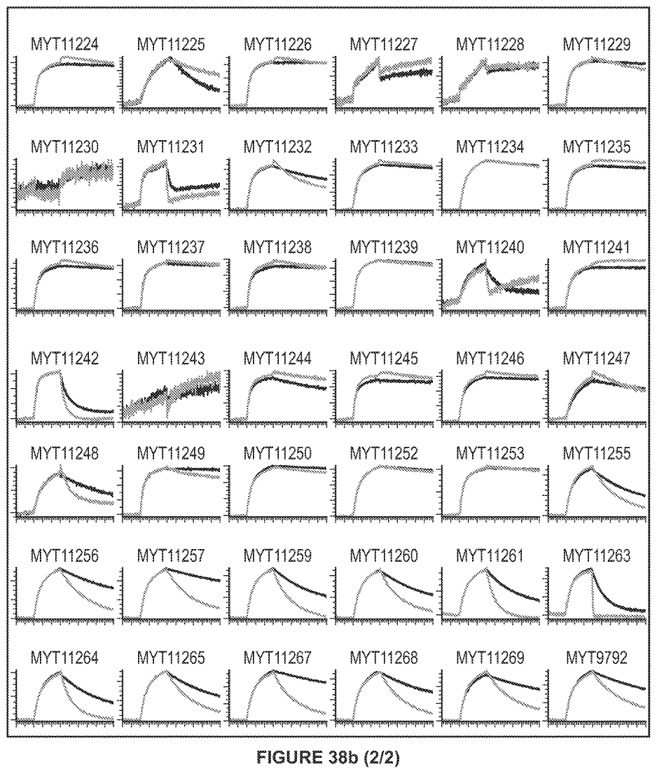

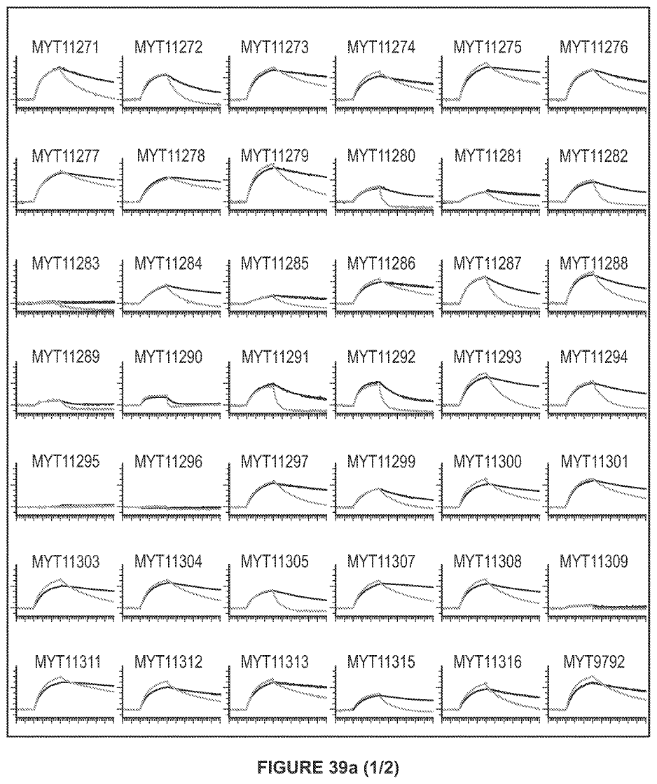

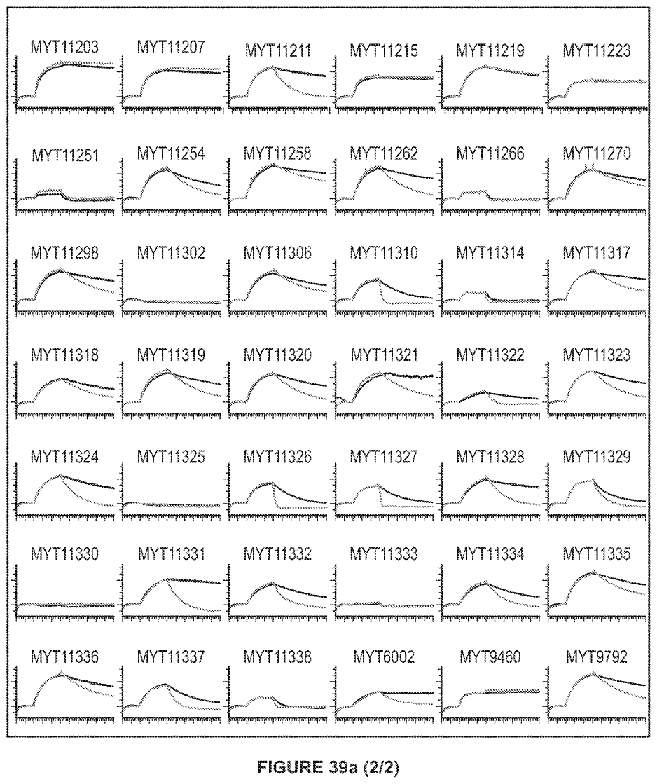

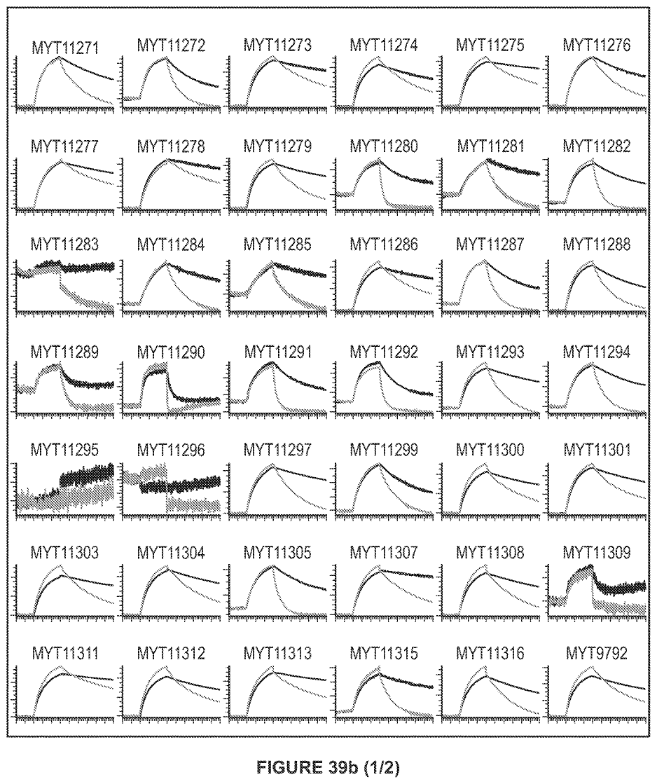

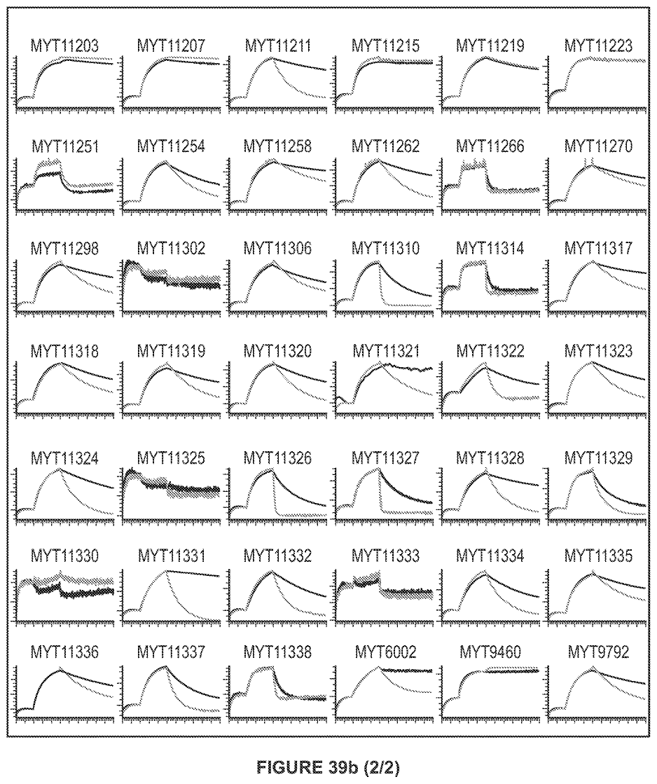

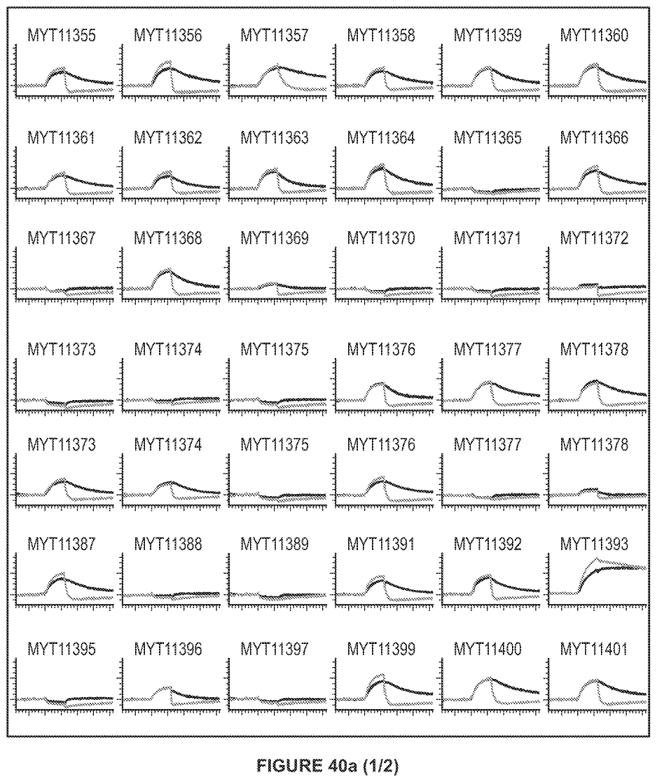

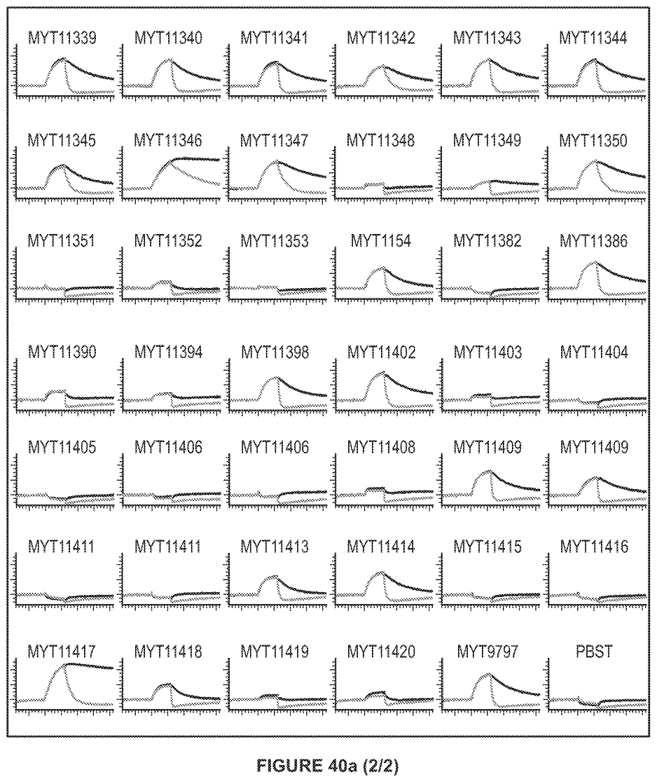

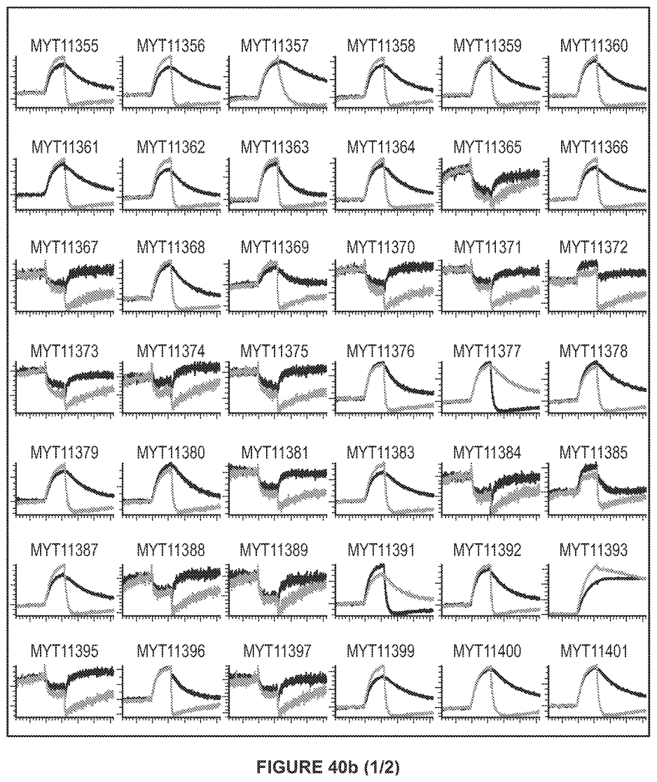

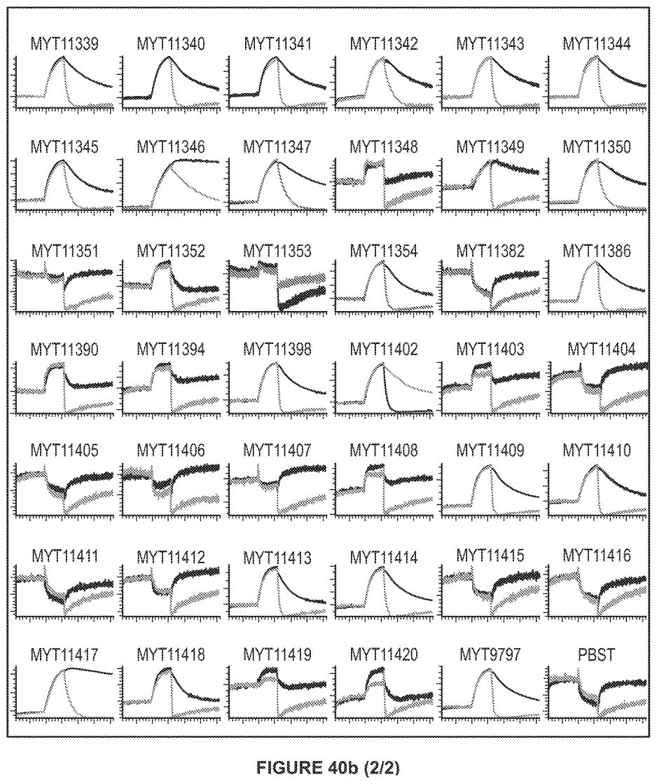

Multiple PTK7-binding monoclonal antibodies have been described in the literature and can be used as a template for engineering pH-dependent binding. We produced seven (7) novel monoclonal antibodies by immunizing rabbits with the ECD of human PTK7, and from hundreds of clones, initially selected MYT9345 (VH SEQ ID NO: 301, VL SEQ ID NO: 5) as a PTK7-binding monoclonal antibody for pH engineering via histidine scanning. Briefly, CDRs in the heavy and light chains were identified using the methods described by Kabat et al (Kabat et al. (1992) Sequences of Proteins of Immunological Interest, DIANE publishing) and IMGT (Lefranc MP (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the heavy and light chain CDRs were systematically substituted with a histidine, one at a time to produce MYT9345 HC variants (MYT10885-MYT10918) and MYT9345 LC variants (MYT10919-MYT10950). In cases where the starting CDR residue was a histidine, it was mutated to an alanine. Antibody variants with only one histidine or alanine mutation in either a heavy or light chain CDR were generated by co-transfection of Expi293 cells with either a) one heavy chain sequence variant, and the corresponding starting ABPC light chain; or b) one light chain sequence variant, and the corresponding starting ABPC heavy chain using methods known to the art. After allowing for four days of protein expression, cell culture supernatants were collected, quantified by SDS-PAGE analysis (DNS), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1× PBST, pH 7.4 for low expressors for loading onto the sensor tips. The resulting diluted supernatants were then captured on an anti-human Fc sensor (Forte Bio). A baseline was established using 1×PBST (50 mM Potassium Phosphate Buffer+150 mM NaCl+0.05% Tween 20) pH 7.4, and the sensor was associated with 50 nM of PTK7 (Human PTK7 Protein, His Tag, Acro Biosystems) in 1×PBST pH 7.4 for 120 sec to generate an association curve. In the dissociation phase, the antibody-antigen complex on the sensor was exposed to 1×PBST pH 7.4 or pH 5.4, for 300 sec. Association and dissociation phase curves were examined for the starting ABPC antibody (with no substitutions) and each corresponding antibody variant at pH 5.4 and pH 7.4 to inform on two criteria: a) enhanced dissociation (i.e., higher koff values) at pH 5.4 due to histidine or alanine substitution compared to the starting ABPC, (with no substitutions), and b) reduced dissociation at pH 7.4 (i.e., lower koff values) compared to pH 5.4 in the antibody variant itself and with the starting ABPC (with no substitutions). Heavy and light chain variants that showed either enhanced dissociation at pH 5.4 or reduced dissociation at pH 7.4 or both (as compared to starting ABPC; see FIGS. 34a-34b) were selected for further analysis (e.g., MYT10888-89, MYT10891, MYT10895-99, MYT10903, MYT10905, MYT10911, MYT10916, MYT10920, MYT10925-28, MYT10930, MYT10935-37, MYT10939, MYT10942-44, MYT10946 and MYT10949). It was also noted that while some mutations obliterated PTK7 binding, others were tolerated with little or no change in PTK7 binding kinetics.

Especially because histidine is a large, positively charged amino acid, heavy or light chain variants exhibiting no significant change in binding were noted as positions in the heavy or light chain that may tolerate a wide range of mutations and lead to antibodies with different sequence but similar binding properties, a designation that is not otherwise apparent.

TABLE 21
MYT9345 variants summary information, including SEQ ID NO
designations, amino acid substitution position information,
and binding data. Under “Binding Result”: 1 =
pH dependent binding; 2 = substantially similar binding;
and 3 = no binding, each relative to parent

| | | | CDR | HCV | LCV | VH | VL |
|---|---|---|---|---|---|---|---|
| Ala Sub Pos | Binding | MYT | Sub | Sub | Sub | SEQ | SEQ |
| (if present) | Result | # | Pos | Pos | Pos | ID | ID |
| | n/a | 9345 | n/a | n/a | n/a | 301 | 302 |
| | 2 | 10885 | H1 01 | 26 | n/a | 309 | 302 |
| | 2 | 10886 | H1 02 | 27 | n/a | 310 | 302 |
| | 2 | 10887 | H1 03 | 28 | n/a | 311 | 302 |
| | 1 | 10888 | H1 04 | 29 | n/a | 312 | 302 |
| | 1 | 10889 | H1 05 | 30 | n/a | 313 | 302 |
| | 2 | 10890 | H1 06 | 31 | n/a | 314 | 302 |
| | 1 | 10891 | H1 07 | 32 | n/a | 315 | 302 |
| | 3 | 10892 | H1 08 | 33 | n/a | 316 | 302 |
| | 2 | 10893 | H1 09 | 34 | n/a | 317 | 302 |
| | 2 | 10894 | H1 10 | 35 | n/a | 318 | 302 |
| | 1 | 10895 | H2 01 | 50 | n/a | 319 | 302 |
| | 1 | 10896 | H2 02 | 51 | n/a | 320 | 302 |
| | 1 | 10897 | H2 03 | 52 | n/a | 321 | 302 |
| | 1 | 10898 | H2 04 | 53 | n/a | 322 | 302 |
| | 1 | 10899 | H2 05 | 54 | n/a | 323 | 302 |
| | 2 | 10900 | H2 06 | 55 | n/a | 324 | 302 |
| | 2 | 10901 | H2 07 | 56 | n/a | 325 | 302 |
| | 2 | 10902 | H2 08 | 57 | n/a | 326 | 302 |
| | 1 | 10903 | H2 09 | 58 | n/a | 327 | 302 |
| | 2 | 10904 | H2 10 | 59 | n/a | 328 | 302 |
| | 1 | 10905 | H2 11 | 60 | n/a | 329 | 302 |
| | 2 | 10906 | H2 12 | 61 | n/a | 330 | 302 |
| | 2 | 10907 | H2 13 | 62 | n/a | 331 | 302 |
| | 2 | 10908 | H2 14 | 63 | n/a | 332 | 302 |
| | 2 | 10909 | H2 15 | 64 | n/a | 333 | 302 |
| | 2 | 10910 | H2 16 | 65 | n/a | 334 | 302 |
| | 1 | 10911 | H3 01 | 96 | n/a | 335 | 302 |
| | 2 | 10912 | H3 02 | 97 | n/a | 336 | 302 |
| | 3 | 10913 | H3 03 | 98 | n/a | 337 | 302 |
| | 2 | 10914 | H3 04 | 99 | n/a | 338 | 302 |
| | 2 | 10915 | H3 05 | 100 | n/a | 339 | 302 |
| | 1 | 10916 | H3 06 | 101 | n/a | 340 | 302 |
| | 1 | 10917 | n/a | n/a | n/a | 341 | 302 |
| A | 1 | 10918 | H2 03 | 52 | n/a | 342 | 302 |
| | 2 | 10919 | L1 01 | n/a | 24 | 301 | 343 |
| | 1 | 10920 | L1 02 | n/a | 25 | 301 | 344 |
| | 2 | 10921 | L1 03 | n/a | 26 | 301 | 345 |
| | 2 | 10922 | L1 04 | n/a | 27 | 301 | 346 |
| | 2 | 10923 | L1 05 | n/a | 28 | 301 | 347 |
| | 2 | 10924 | L1 06 | n/a | 29 | 301 | 348 |
| | 1 | 10925 | L1 07 | n/a | 30 | 301 | 349 |
| | 1 | 10926 | L1 08 | n/a | 31 | 301 | 350 |
| | 1 | 10927 | L1 09 | n/a | 32 | 301 | 351 |
| | 1 | 10928 | L1 10 | n/a | 33 | 301 | 352 |
| | 2 | 10929 | L1 11 | n/a | 34 | 301 | 353 |
| | 1 | 10930 | L1 12 | n/a | 35 | 301 | 354 |
| | 2 | 10931 | L1 13 | n/a | 36 | 301 | 355 |
| | 2 | 10932 | L2 01 | n/a | 52 | 301 | 356 |
| | 2 | 10933 | L2 02 | n/a | 53 | 301 | 357 |
| | 2 | 10934 | L2 03 | n/a | 54 | 301 | 358 |
| | 1 | 10935 | L2 04 | n/a | 55 | 301 | 359 |
| | 1 | 10936 | L2 05 | n/a | 56 | 301 | 360 |
| | 1 | 10937 | L2 06 | n/a | 57 | 301 | 361 |
| | 2 | 10938 | L2 07 | n/a | 58 | 301 | 362 |
| | 1 | 10939 | L3 01 | n/a | 91 | 301 | 363 |
| | 2 | 10940 | L3 02 | n/a | 92 | 301 | 364 |
| | 2 | 10941 | L3 03 | n/a | 93 | 301 | 365 |
| | 1 | 10942 | L3 04 | n/a | 94 | 301 | 366 |
| | 1 | 10943 | L3 05 | n/a | 95 | 301 | 367 |
| | 1 | 10944 | L3 06 | n/a | 96 | 301 | 368 |
| | 2 | 10945 | L3 07 | n/a | 97 | 301 | 369 |
| | 1 | 10946 | L3 08 | n/a | 98 | 301 | 370 |
| | 2 | 10947 | L3 09 | n/a | 99 | 301 | 371 |
| | 2 | 10948 | L3 10 | n/a | 100 | 301 | 372 |
| | 1 | 10949 | n/a | n/a | n/a | 301 | 373 |
| A | 1 | 10950 | L3 05 | n/a | 95 | 301 | 374 |

The methods of this Example were systematically applied to MYT9359 (HCV SEQ ID NO: 375, LCV SEQ ID NO: 79); MYT9361 (HCV SEQ ID NO: 459, LCV SEQ ID NO: 163); MYT9412 (HCV SEQ ID NO: 542, LCV SEQ ID NO: 543); MYT9460 (HCV SEQ ID NO: 628, LCV SEQ ID NO: 629); MYT9792 (HCV SEQ ID NO: 710, LCV SEQ ID NO: 711); and MYT9797 (HCV SEQ ID NO: 803, LCV SEQ ID NO: 804).

As before, to generate pH-dependent sequence variants, individual amino acid residues within the heavy and light chain CDRs were systematically substituted with a histidine (or alanine if a histidine was already present), one at a time to produce MYT9359 HC variants (MYT10951-MYT10991), MYT9359 LC variants (MYT10992-MYT11026); MYT9361 HC variants (MYT11027-MYT11071), MYT9361 LC variants (MYT11072-MYT11101); MYT9412 HC variants (MYT11102-MYT11144), MYT9412 LC variants (MYT11145-MYT11179); MYT9460 HC variants (MYT11180-MYT11219), MYT9460 LC variants (MYT11220-MYT11253); MYT9792 HC variants (MYT11254-MYT11303), MYT9792 LC variants (MYT11304-MYT11338); and MYT9797 HC variants (MYT11339-MYT11382), MYT9797 LC variants (MYT11383-MYT11420).

Variants that show either enhanced dissociation at pH 5.4 or reduced dissociation at pH 7.4, or both are selected for further analysis. (e.g., MYT10951-52, MYT10955, MYT10957, MYT10959, MYT10962-63, MYT10966, MYT10969-70, MYT10974, MYT10978, MYT10980, MYT10989, MYT10993, MYT10997-99, MYT11000-1, MYT11003-6, MYT11010, MYT11012-14, MYT11017-18, MYT11021, MYT11023, MYT11056, MYT11058-59, MYT11063 and MYT11068, MYT11033, MYT11056, MYT11058, MYT11059, MYT11063, MYT11068, MYT11078, MYT11079, MYT11081, MYT11084, MYT11093 and MYT11096, MYT11103, MYT11105-08, MYT11115, MYT11117, MYT11120, MYT11134, MYT11136-37, MYT11146, MYT11150-53, MYT11163, MYT11166 and MYT11172, MYT11180, MYT11182, MYT11184, MYT11190, MYT11192-97, MYT11210-13, MYT11221-22, MYT11224-25, MYT11229, MYT11232, MYT11236, MYT11238, MYT11240, MYT11244-49, MYT11257-59, MYT11261, MYT11263, MYT11264, MYT11267, MYT11271-72, MYT11279-82, MYT11284-85, MYT11287, MYT11289, MYT11291-94, MYT11297, MYT11305, MYT11307, MYT11309-10, MYT11315, MYT11316, MYT11321-22, MYT11324, MYT11326-28, MYT11331 and MYT11332, and MYT11346, MYT11349, MYT11352, MYT11354-57, MYT11363, MYT11369, MYT11376, MYT11390, MYT11392-94, MYT11396, MYT11398, MYT11409, and MYT11410). 35a-35b, 36a-36b, 37a-37b, 38a-38b, 39a-39b, and 40a-40b present octet plots for these variants and their parental antibodies.

Heavy and light chain combination variants showing either enhanced dissociation at pH 5.4 or reduced dissociation at pH 7.4 or both (as compared to the starting ABPC), may be selected for further analysis. Particularly desirable ABPC variants demonstrate both pH-dependent binding and increased, target-specific uptake by PTK7+ cells.

TABLE 22
MYT9359 variants summary information, including SEQ ID NO
designations, amino acid substitution position information,
and binding data. Under “Binding Result”: 1 =
pH dependent binding; 2 = substantially similar binding;
and 3 = no binding, each relative to parent

| | | | CDR | HCV | LCV | VH | VL |
|---|---|---|---|---|---|---|---|
| Ala Sub Pos | Binding | MYT | Sub | Sub | Sub | SEQ | SEQ |
| (if present) | Result | # | Pos | Pos | Pos | ID | ID |
| | n/a | 9359 | n/a | n/a | n/a | 375 | 376 |
| | 1 | 10951 | H1 01 | 26 | n/a | 383 | 376 |
| | 1 | 10952 | H1 02 | 27 | n/a | 384 | 376 |
| | 2 | 10953 | H1 03 | 28 | n/a | 385 | 376 |
| | 2 | 10954 | H1 04 | 29 | n/a | 386 | 376 |
| | 1 | 10955 | H1 05 | 30 | n/a | 387 | 376 |
| | 2 | 10956 | H1 06 | 31 | n/a | 388 | 376 |
| | 1 | 10957 | H1 07 | 32 | n/a | 389 | 376 |
| | 3 | 10958 | H1 08 | 33 | n/a | 390 | 376 |
| | 1 | 10959 | H1 09 | 34 | n/a | 391 | 376 |
| | 3 | 10960 | H1 10 | 35 | n/a | 392 | 376 |
| | 3 | 10961 | H2 01 | 50 | n/a | 393 | 376 |
| | 1 | 10962 | H2 02 | 51 | n/a | 394 | 376 |
| | 1 | 10963 | H2 03 | 52 | n/a | 395 | 376 |
| | 3 | 10964 | H2 04 | 53 | n/a | 396 | 376 |
| | 2 | 10965 | H2 05 | 54 | n/a | 397 | 376 |
| | 1 | 10966 | H2 06 | 55 | n/a | 398 | 376 |
| | 2 | 10967 | H2 07 | 56 | n/a | 399 | 376 |
| | 2 | 10968 | H2 08 | 57 | n/a | 400 | 376 |
| | 1 | 10969 | H2 09 | 58 | n/a | 401 | 376 |
| | 1 | 10970 | H2 10 | 59 | n/a | 402 | 376 |
| | 2 | 10971 | H2 11 | 60 | n/a | 403 | 376 |
| | 2 | 10972 | H2 12 | 61 | n/a | 404 | 376 |
| | 2 | 10973 | H2 13 | 62 | n/a | 405 | 376 |
| | 1 | 10974 | H2 14 | 63 | n/a | 406 | 376 |
| | 2 | 10975 | H2 15 | 64 | n/a | 407 | 376 |
| | 2 | 10976 | H2 16 | 65 | n/a | 408 | 376 |
| | 2 | 10977 | H3 01 | 96 | n/a | 409 | 376 |
| | 1 | 10978 | H3 02 | 97 | n/a | 410 | 376 |
| | 3 | 10979 | H3 03 | 98 | n/a | 411 | 376 |
| | 1 | 10980 | H3 04 | 99 | n/a | 412 | 376 |
| | 3 | 10981 | H3 05 | 100 | n/a | 413 | 376 |
| | 3 | 10982 | H3 06 | 101 | n/a | 414 | 376 |
| | 3 | 10983 | H3 07 | 102 | n/a | 415 | 376 |
| | 3 | 10984 | H3 08 | 103 | n/a | 416 | 376 |
| | 3 | 10985 | H3 09 | 104 | n/a | 417 | 376 |
| | 3 | 10986 | H3 10 | 105 | n/a | 418 | 376 |
| | 2 | 10987 | H3 11 | 106 | n/a | 419 | 376 |
| | 2 | 10988 | H3 12 | 107 | n/a | 420 | 376 |
| | 1 | 10989 | H3 13 | 108 | n/a | 421 | 376 |
| | 2 | 10990 | | | n/a | 422 | 376 |
| A | 1 | 10991 | H3 12 | 107 | n/a | 423 | 376 |
| | 2 | 10992 | L1 01 | n/a | 24 | 375 | 424 |
| | 1 | 10993 | L1 02 | n/a | 25 | 375 | 425 |
| | 2 | 10994 | L1 03 | n/a | 26 | 375 | 426 |
| | 2 | 10995 | L1 04 | n/a | 27 | 375 | 427 |
| | 2 | 10996 | L1 05 | n/a | 28 | 375 | 428 |
| | 1 | 10997 | L1 06 | n/a | 29 | 375 | 429 |
| | 1 | 10998 | L1 07 | n/a | 30 | 375 | 430 |
| | 1 | 10999 | L1 08 | n/a | 31 | 375 | 431 |
| | 1 | 11000 | L1 09 | n/a | 32 | 375 | 432 |
| | 1 | 11001 | L1 10 | n/a | 33 | 375 | 433 |
| | 3 | 11002 | L1 11 | n/a | 34 | 375 | 434 |
| | 1 | 11003 | L2 01 | n/a | 50 | 375 | 435 |
| | 1 | 11004 | L2 02 | n/a | 51 | 375 | 436 |
| | 1 | 11005 | L2 03 | n/a | 52 | 375 | 437 |
| | 1 | 11006 | L2 04 | n/a | 53 | 375 | 438 |
| | 2 | 11007 | L2 05 | n/a | 54 | 375 | 439 |
| | 2 | 11008 | L2 06 | n/a | 55 | 375 | 440 |
| | 2 | 11009 | L2 07 | n/a | 56 | 375 | 441 |
| | 1 | 11010 | L3 01 | n/a | 89 | 375 | 442 |
| | 3 | 11011 | L3 02 | n/a | 90 | 375 | 443 |
| | 1 | 11012 | L3 03 | n/a | 91 | 375 | 444 |
| | 1 | 11013 | L3 04 | n/a | 92 | 375 | 445 |
| | 1 | 11014 | L3 05 | n/a | 93 | 375 | 446 |
| | 2 | 11015 | L3 06 | n/a | 94 | 375 | 447 |
| | 2 | 11016 | L3 07 | n/a | 95 | 375 | 448 |
| | 1 | 11017 | L3 08 | n/a | 96 | 375 | 449 |
| | 1 | 11018 | L3 09 | n/a | 97 | 375 | 450 |
| | 2 | 11019 | L3 10 | n/a | 98 | 375 | 451 |
| | 2 | 11020 | L3 11 | n/a | 99 | 375 | 452 |
| | 1 | 11021 | L3 12 | n/a | 100 | 375 | 453 |
| | 3 | 11022 | L3 13 | n/a | 101 | 375 | 454 |
| | 1 | 11023 | L3 14 | n/a | 102 | 375 | 455 |
| | 1 | 11024 | | n/a | 103 | 375 | 456 |
| A | 1 | 11025 | L3 10 | n/a | 98 | 375 | 457 |
| A | 1 | 11026 | L3 13 | n/a | 101 | 375 | 458 |

TABLE 23
MYT9361 variants summary information, including SEQ ID NO
designations, amino acid substitution position information,
and binding data. Under “Binding Result”: 1 =
pH dependent binding; 2 = substantially similar binding;
and 3 = no binding, each relative to parent

| | | | CDR | HCV | LCV | VH | VL |
|---|---|---|---|---|---|---|---|
| Ala Sub Pos | Binding | MYT | Sub | Sub | Sub | SEQ | SEQ |
| (if present) | Result | # | Pos | Pos | Pos | ID | ID |
| | n/a | 9361 | n/a | n/a | n/a | 459 | 460 |
| | 2 | 11027 | H1 01 | 26 | n/a | 467 | 460 |
| | 2 | 11028 | H1 02 | 27 | n/a | 468 | 460 |
| | 2 | 11029 | H1 03 | 28 | n/a | 469 | 460 |
| | 2 | 11030 | H1 04 | 29 | n/a | 470 | 460 |
| | 2 | 11031 | H1 05 | 30 | n/a | 471 | 460 |
| | 2 | 11032 | H1 06 | 31 | n/a | 472 | 460 |
| | 1 | 11033 | H1 07 | 32 | n/a | 473 | 460 |
| | 2 | 11034 | H1 08 | 33 | n/a | 474 | 460 |
| | 2 | 11035 | H1 09 | 34 | n/a | 475 | 460 |
| | 2 | 11036 | H1 10 | 35 | n/a | 476 | 460 |
| | 2 | 11037 | H2 01 | 50 | n/a | 477 | 460 |
| | 2 | 11038 | H2 02 | 51 | n/a | 478 | 460 |
| | 2 | 11039 | H2 03 | 52 | n/a | 479 | 460 |
| | 2 | 11040 | H2 04 | 53 | n/a | 480 | 460 |
| | 2 | 11041 | H2 05 | 54 | n/a | 481 | 460 |
| | 2 | 11042 | H2 06 | 55 | n/a | 482 | 460 |
| | 2 | 11043 | H2 07 | 56 | n/a | 483 | 460 |
| | 2 | 11044 | H2 08 | 57 | n/a | 484 | 460 |
| | 2 | 11045 | H2 09 | 58 | n/a | 485 | 460 |
| | 2 | 11046 | H2 10 | 59 | n/a | 486 | 460 |
| | 2 | 11047 | H2 11 | 60 | n/a | 487 | 460 |
| | 2 | 11048 | H2 12 | 61 | n/a | 488 | 460 |
| | 2 | 11049 | H2 13 | 62 | n/a | 489 | 460 |
| | 2 | 11050 | H2 14 | 63 | n/a | 490 | 460 |
| | 2 | 11051 | H2 15 | 64 | n/a | 491 | 460 |
| | 2 | 11052 | H2 16 | 65 | n/a | 492 | 460 |
| | 1 | 11053 | | | n/a | 493 | 460 |
| | 2 | 11054 | | | n/a | 494 | 460 |
| | 2 | 11055 | H3 01 | 98 | n/a | 495 | 460 |
| | 1 | 11056 | H3 02 | 99 | n/a | 496 | 460 |
| | 2 | 11057 | H3 03 | 100 | n/a | 497 | 460 |
| | 1 | 11058 | H3 04 | 101 | n/a | 498 | 460 |
| | 1 | 11059 | H3 05 | 102 | n/a | 499 | 460 |
| | 2 | 11060 | H3 06 | 103 | n/a | 500 | 460 |
| | 2 | 11061 | H3 07 | 104 | n/a | 501 | 460 |
| | 2 | 11062 | H3 08 | 105 | n/a | 502 | 460 |
| | 1 | 11063 | H3 09 | 106 | n/a | 503 | 460 |
| | 2 | 11064 | H3 10 | 107 | n/a | 504 | 460 |
| | 2 | 11065 | H3 11 | 108 | n/a | 505 | 460 |
| | 2 | 11066 | H3 12 | 109 | n/a | 506 | 460 |
| | 2 | 11067 | H3 13 | 110 | n/a | 507 | 460 |
| | 1 | 11068 | H3 14 | 111 | n/a | 508 | 460 |
| | 1 | 11069 | | | | 509 | 460 |
| A | 2 | 11070 | H3 02 | 99 | n/a | 510 | 460 |
| A | 2 | 11071 | H3 03 | 100 | n/a | 511 | 460 |
| | 2 | 11072 | L1 01 | n/a | 24 | 459 | 512 |
| | 2 | 11073 | L1 02 | n/a | 25 | 459 | 513 |
| | 2 | 11074 | L1 03 | n/a | 26 | 459 | 514 |
| | 2 | 11075 | L1 04 | n/a | 27 | 459 | 515 |
| | 2 | 11076 | L1 05 | n/a | 28 | 459 | 516 |
| | 3 | 11077 | L1 06 | n/a | 29 | 459 | 517 |
| | 1 | 11078 | L1 07 | n/a | 30 | 459 | 518 |
| | 1 | 11079 | L1 08 | n/a | 31 | 459 | 519 |
| | 2 | 11080 | L1 09 | n/a | 32 | 459 | 520 |
| | 1 | 11081 | L1 10 | n/a | 33 | 459 | 521 |
| | 3 | 11082 | L1 11 | n/a | 34 | 459 | 522 |
| | 2 | 11083 | L2 01 | n/a | 50 | 459 | 523 |
| | 1 | 11084 | L2 02 | n/a | 51 | 459 | 524 |
| | 2 | 11085 | L2 03 | n/a | 52 | 459 | 525 |
| | 2 | 11086 | L2 04 | n/a | 53 | 459 | 526 |
| | 2 | 11087 | L2 05 | n/a | 54 | 459 | 527 |
| | 2 | 11088 | L2 06 | n/a | 55 | 459 | 528 |
| | 2 | 11089 | L2 07 | n/a | 56 | 459 | 529 |
| | 2 | 11090 | L3 01 | n/a | 89 | 459 | 530 |
| | 3 | 11091 | L3 02 | n/a | 90 | 459 | 531 |
| | 2 | 11092 | L3 03 | n/a | 91 | 459 | 532 |
| | 1 | 11093 | L3 04 | n/a | 92 | 459 | 533 |
| | 3 | 11094 | L3 05 | n/a | 93 | 459 | 534 |
| | 2 | 11095 | L3 06 | n/a | 94 | 459 | 535 |
| | 1 | 11096 | L3 07 | n/a | 95 | 459 | 536 |
| | 2 | 11097 | L3 08 | n/a | 96 | 459 | 537 |
| | 2 | 11098 | L3 09 | n/a | 97 | 459 | 538 |
| | 2 | 11099 | L3 10 | n/a | 98 | 459 | 539 |
| | 1 | 11100 | | | | 459 | 540 |
| A | 3 | 11101 | L3 05 | n/a | 93 | 459 | 541 |

TABLE 24
MYT9412 variants summary information, including SEQ ID NO
designations, amino acid substitution position information,
and binding data. Under “Binding Result”: 1 =
pH dependent binding; 2 = substantially similar binding;
and 3 = no binding, each relative to parent

| | | | CDR | HCV | LCV | VH | |
|---|---|---|---|---|---|---|---|
| Ala Sub Pos | Binding | MYT | Sub | Sub | Sub | SEQ | VL |
| (if present) | Result | # | Pos | Pos | Pos | IDID | SEQ |
| | n/a | 9412 | n/a | n/a | n/a | 542 | 543 |
| | 2 | 11102 | H1 01 | 26 | n/a | 550 | 543 |
| | 1 | 11103 | H1 02 | 27 | n/a | 551 | 543 |
| | 2 | 11104 | H1 03 | 28 | n/a | 552 | 543 |
| | 1 | 11105 | H1 04 | 29 | n/a | 553 | 543 |
| | 1 | 11106 | H1 05 | 30 | n/a | 554 | 543 |
| | 1 | 11107 | H1 06 | 31 | n/a | 555 | 543 |
| | 1 | 11108 | H1 07 | 32 | n/a | 556 | 543 |
| | 2 | 11109 | H1 08 | 33 | n/a | 557 | 543 |
| | 2 | 11110 | H1 09 | 34 | n/a | 558 | 543 |
| | 3 | 11111 | H1 10 | 35 | n/a | 559 | 543 |
| | 3 | 11112 | H2 01 | 50 | n/a | 560 | 543 |
| | 2 | 11113 | H2 02 | 51 | n/a | 561 | 543 |
| | 3 | 11114 | H2 03 | 52 | n/a | 562 | 543 |
| | 1 | 11115 | H2 04 | 53 | n/a | 563 | 543 |
| | 2 | 11116 | H2 05 | 54 | n/a | 564 | 543 |
| | 1 | 11117 | H2 06 | 55 | n/a | 565 | 543 |
| | 2 | 11118 | H2 07 | 56 | n/a | 566 | 543 |
| | 2 | 11119 | H2 08 | 57 | n/a | 567 | 543 |
| | 1 | 11120 | H2 09 | 58 | n/a | 568 | 543 |
| | 2 | 11121 | H2 10 | 59 | n/a | 569 | 543 |
| | 2 | 11122 | H2 11 | 60 | n/a | 570 | 543 |
| | 2 | 11123 | H2 12 | 61 | n/a | 571 | 543 |
| | 2 | 11124 | H2 13 | 62 | n/a | 572 | 543 |
| | 2 | 11125 | H2 14 | 63 | n/a | 573 | 543 |
| | 2 | 11126 | H2 15 | 64 | n/a | 574 | 543 |
| | 2 | 11127 | H2 16 | 65 | n/a | 575 | 543 |
| | 3 | 11128 | H3 01 | 96 | n/a | 576 | 543 |
| | 2 | 11129 | H3 02 | 97 | n/a | 577 | 543 |
| | 3 | 11130 | H3 03 | 98 | n/a | 578 | 543 |
| | 3 | 11131 | H3 04 | 99 | n/a | 579 | 543 |
| | 3 | 11132 | H3 05 | 100 | n/a | 580 | 543 |
| | 3 | 11133 | H3 06 | 101 | n/a | 581 | 543 |
| | 1 | 11134 | H3 07 | 102 | n/a | 582 | 543 |
| | 2 | 11135 | H3 08 | 103 | n/a | 583 | 543 |
| | 1 | 11136 | H3 09 | 104 | n/a | 584 | 543 |
| | 1 | 11137 | H3 10 | 105 | n/a | 585 | 543 |
| | 2 | 11138 | H3 11 | 106 | n/a | 586 | 543 |
| | 2 | 11139 | H3 12 | 107 | n/a | 587 | 543 |
| | 2 | 11140 | H3 13 | 108 | n/a | 588 | 543 |
| | 2 | 11141 | H3 14 | 109 | n/a | 589 | 543 |
| | 1 | 11142 | | | n/a | 590 | 543 |
| A | 1 | 11143 | H2 06 | 55 | n/a | 591 | 543 |
| A | 1 | 11144 | H3 03 | 98 | n/a | 592 | 543 |
| | 2 | 11145 | L1 01 | n/a | 24 | 542 | 593 |
| | 1 | 11146 | L1 02 | n/a | 25 | 542 | 594 |
| | 2 | 11147 | L1 03 | n/a | 26 | 542 | 595 |
| | 2 | 11148 | L1 04 | n/a | 27 | 542 | 596 |
| | 2 | 11149 | L1 05 | n/a | 28 | 542 | 597 |
| | 1 | 11150 | L1 06 | n/a | 29 | 542 | 598 |
| | 1 | 11151 | L1 07 | n/a | 30 | 542 | 599 |
| | 1 | 11152 | L1 08 | n/a | 31 | 542 | 600 |
| | 1 | 11153 | L1 09 | n/a | 32 | 542 | 601 |
| | 2 | 11154 | L1 10 | n/a | 33 | 542 | 602 |
| | 2 | 11155 | L1 11 | n/a | 34 | 542 | 603 |
| | 2 | 11156 | L2 01 | n/a | 50 | 542 | 604 |
| | 2 | 11157 | L2 02 | n/a | 51 | 542 | 605 |
| | 2 | 11158 | L2 03 | n/a | 52 | 542 | 606 |
| | 2 | 11159 | L2 04 | n/a | 53 | 542 | 607 |
| | 2 | 11160 | L2 05 | n/a | 54 | 542 | 608 |
| | 2 | 11161 | L2 06 | n/a | 55 | 542 | 609 |
| | 2 | 11162 | L2 07 | n/a | 56 | 542 | 610 |
| | 1 | 11163 | L3 01 | n/a | 89 | 542 | 611 |
| | 2 | 11164 | L3 02 | n/a | 90 | 542 | 612 |
| | 3 | 11165 | L3 03 | n/a | 91 | 542 | 613 |
| | 1 | 11166 | L3 04 | n/a | 92 | 542 | 614 |
| | 2 | 11167 | L3 05 | n/a | 93 | 542 | 615 |
| | 2 | 11168 | L3 06 | n/a | 94 | 542 | 616 |
| | 2 | 11169 | L3 07 | n/a | 95 | 542 | 617 |
| | 2 | 11170 | L3 08 | n/a | 96 | 542 | 618 |
| | 2 | 11171 | L3 09 | n/a | 97 | 542 | 619 |
| | 1 | 11172 | L3 10 | n/a | 98 | 542 | 620 |
| | 2 | 11173 | L3 11 | n/a | 99 | 542 | 621 |
| | 3 | 11174 | L3 12 | n/a | 100 | 542 | 622 |
| | 2 | 11175 | L3 13 | n/a | 101 | 542 | 623 |
| | 3 | 11176 | | n/a | | 542 | 624 |
| A | 1 | 11177 | L3 10 | n/a | 98 | 542 | 625 |
| A | 1 | 11178 | L3 11 | n/a | 99 | 542 | 626 |
| A | 2 | 11179 | L3 03 | n/a | 91 | 542 | 627 |

TABLE 25
MYT9460 variants summary information, including SEQ ID NO
designations, amino acid substitution position information,
and binding data. Under “Binding Result”: 1 =
pH dependent binding; 2 = substantially similar binding;
and 3 = no binding, each relative to parent

| | | | CDR | HCV | LCV | VH | VL |
|---|---|---|---|---|---|---|---|
| Ala Sub Pos | Binding | MYT | Sub | Sub | Sub | SEQ | SEQ |
| (if present) | Result | # | Pos | Pos | Pos | ID | ID |
| | n/a | 9460 | n/a | n/a | n/a | 628 | 629 |
| | 1 | 11180 | H1 01 | 26 | n/a | 636 | 629 |
| | 2 | 11181 | H1 02 | 27 | n/a | 637 | 629 |
| | 1 | 11182 | H1 03 | 28 | n/a | 638 | 629 |
| | 2 | 11183 | H1 04 | 29 | n/a | 639 | 629 |
| | 1 | 11184 | H1 05 | 30 | n/a | 640 | 629 |
| | 2 | 11185 | H1 06 | 31 | n/a | 641 | 629 |
| | 2 | 11186 | H1 07 | 32 | n/a | 642 | 629 |
| | 2 | 11187 | H1 08 | 33 | n/a | 643 | 629 |
| | 2 | 11188 | H1 09 | 34 | n/a | 644 | 629 |
| | 3 | 11189 | H1 10 | 35 | n/a | 645 | 629 |
| | 1 | 11190 | H2 01 | 50 | n/a | 646 | 629 |
| | 3 | 11191 | H2 02 | 51 | n/a | 647 | 629 |
| | 1 | 11192 | H2 03 | 52 | n/a | 648 | 629 |
| | 1 | 11193 | H2 04 | 53 | n/a | 649 | 629 |
| | 1 | 11194 | H2 05 | 54 | n/a | 650 | 629 |
| | 1 | 11195 | H2 06 | 55 | n/a | 651 | 629 |
| | 1 | 11196 | H2 07 | 56 | n/a | 652 | 629 |
| | 1 | 11197 | H2 08 | 57 | n/a | 653 | 629 |
| | 2 | 11198 | H2 09 | 58 | n/a | 654 | 629 |
| | 2 | 11199 | H2 10 | 59 | n/a | 655 | 629 |
| | 2 | 11200 | H2 11 | 60 | n/a | 656 | 629 |
| | 2 | 11201 | H2 12 | 61 | n/a | 657 | 629 |
| | 2 | 11202 | H2 13 | 62 | n/a | 658 | 629 |
| | 2 | 11203 | H2 14 | 63 | n/a | 659 | 629 |
| | 3 | 11204 | H2 15 | 64 | n/a | 660 | 629 |
| | 2 | 11205 | H2 16 | 65 | n/a | 661 | 629 |
| | 2 | 11206 | H3 01 | 96 | n/a | 662 | 629 |
| | 2 | 11207 | H3 02 | 97 | n/a | 663 | 629 |
| | 2 | 11208 | H3 03 | 98 | n/a | 664 | 629 |
| | 2 | 11209 | H3 04 | 99 | n/a | 665 | 629 |
| | 1 | 11210 | H3 05 | 100 | n/a | 666 | 629 |
| | 1 | 11211 | H3 06 | 101 | n/a | 667 | 629 |
| | 1 | 11212 | H3 07 | 102 | n/a | 668 | 629 |
| | 1 | 11213 | H3 08 | 103 | n/a | 669 | 629 |
| | 2 | 11214 | H3 09 | 104 | n/a | 670 | 629 |
| | 2 | 11215 | H3 10 | 105 | n/a | 671 | 629 |
| | 2 | 11216 | H3 11 | 106 | n/a | 672 | 629 |
| | 2 | 11217 | | | n/a | 673 | 629 |
| | 2 | 11218 | H1 03 | 28 | n/a | 674 | 629 |
| | 2 | 11219 | H3 05 | 101 | n/a | 675 | 629 |
| | 2 | 11220 | L1 01 | n/a | 24 | 628 | 676 |
| | 1 | 11221 | L1 02 | n/a | 25 | 628 | 677 |
| | 1 | 11222 | L1 03 | n/a | 26 | 628 | 678 |
| | 2 | 11223 | L1 04 | n/a | 27 | 628 | 679 |
| | 1 | 11224 | L1 05 | n/a | 28 | 628 | 680 |
| | 1 | 11225 | L1 06 | n/a | 29 | 628 | 681 |
| | 2 | 11226 | L1 07 | n/a | 30 | 628 | 682 |
| | 2 | 11227 | L1 08 | n/a | 31 | 628 | 683 |
| | 2 | 11228 | L1 09 | n/a | 32 | 628 | 684 |
| | 1 | 11229 | L1 10 | n/a | 33 | 628 | 685 |
| | 3 | 11230 | L1 11 | n/a | 34 | 628 | 686 |
| | 2 | 11231 | L2 01 | n/a | 50 | 628 | 687 |
| | 1 | 11232 | L2 02 | n/a | 51 | 628 | 688 |
| | 2 | 11233 | L2 03 | n/a | 52 | 628 | 689 |
| | 2 | 11234 | L2 04 | n/a | 53 | 628 | 690 |
| | 2 | 11235 | L2 05 | n/a | 54 | 628 | 691 |
| | 1 | 11236 | L2 06 | n/a | 55 | 628 | 692 |
| | 2 | 11237 | L2 07 | n/a | 56 | 628 | 693 |
| | 1 | 11238 | L3 01 | n/a | 89 | 628 | 694 |
| | 2 | 11239 | L3 02 | n/a | 90 | 628 | 695 |
| | 1 | 11240 | L3 03 | n/a | 91 | 628 | 696 |
| | 2 | 11241 | L3 04 | n/a | 92 | 628 | 697 |
| | 2 | 11242 | L3 05 | n/a | 93 | 628 | 698 |
| | 3 | 11243 | L3 06 | n/a | 94 | 628 | 699 |
| | 1 | 11244 | L3 07 | n/a | 95 | 628 | 700 |
| | 1 | 11245 | L3 08 | n/a | 96 | 628 | 701 |
| | 1 | 11246 | L3 09 | n/a | 97 | 628 | 702 |
| | 1 | 11247 | L3 10 | n/a | 98 | 628 | 703 |
| | 1 | 11248 | L3 11 | n/a | 99 | 628 | 704 |
| | 1 | 11249 | L3 12 | n/a | 100 | 628 | 705 |
| | 1 | 11250 | | n/a | | 628 | 706 |
| A | 2 | 11251 | L2 01 | n/a | 50 | 628 | 707 |
| A | 2 | 11252 | L2 04 | n/a | 53 | 628 | 708 |
| A | 1 | 11253 | L3 08 | n/a | 96 | 628 | 709 |

TABLE 26
MYT9792 variants summary information, including SEQ ID NO
designations, amino acid substitution position information,
and binding data. Under “Binding Result”: 1 =
pH dependent binding; 2 = substantially similar binding;
and 3 = no binding, each relative to parent

| | | | CDR | HCV | LCV | VH | VL |
|---|---|---|---|---|---|---|---|
| Ala Sub Pos | Binding | MYT | Sub | Sub | Sub | SEQ | SEQ |
| (if present) | Result | # | Pos | Pos | Pos | ID | ID |
| | n/a | 9792 | n/a | n/a | n/a | 710 | 711 |
| | 2 | 11254 | H1 01 | 26 | n/a | 718 | 711 |
| | 2 | 11255 | H1 02 | 27 | n/a | 719 | 711 |
| | 2 | 11256 | H1 03 | 28 | n/a | 720 | 711 |
| | 1 | 11257 | H1 04 | 29 | n/a | 721 | 711 |
| | 1 | 11258 | H1 05 | 30 | n/a | 722 | 711 |
| | 1 | 11259 | H1 06 | 31 | n/a | 723 | 711 |
| | 2 | 11260 | H1 07 | 32 | n/a | 724 | 711 |
| | 1 | 11261 | H1 08 | 33 | n/a | 725 | 711 |
| | 2 | 11262 | H1 09 | 34 | n/a | 726 | 711 |
| | 1 | 11263 | H1 10 | 35 | n/a | 727 | 711 |
| | 1 | 11264 | H2 01 | 50 | n/a | 728 | 711 |
| | 2 | 11265 | H2 02 | 51 | n/a | 729 | 711 |
| | 2 | 11266 | H2 03 | 52 | n/a | 730 | 711 |
| | 1 | 11267 | H2 04 | 53 | n/a | 731 | 711 |
| | 2 | 11268 | H2 05 | 54 | n/a | 732 | 711 |
| | 2 | 11269 | H2 06 | 55 | n/a | 733 | 711 |
| | 2 | 11270 | H2 07 | 56 | n/a | 734 | 711 |
| | 1 | 11271 | H2 08 | 57 | n/a | 735 | 711 |
| | 1 | 11272 | H2 09 | 58 | n/a | 736 | 711 |
| | 2 | 11273 | H2 10 | 59 | n/a | 737 | 711 |
| | 2 | 11274 | H2 11 | 60 | n/a | 738 | 711 |
| | 2 | 11275 | H2 12 | 61 | n/a | 739 | 711 |
| | 2 | 11276 | H2 13 | 62 | n/a | 740 | 711 |
| | 2 | 11277 | H2 14 | 63 | n/a | 741 | 711 |
| | 2 | 11278 | H2 15 | 64 | n/a | 742 | 711 |
| | 1 | 11279 | H2 16 | 65 | n/a | 743 | 711 |
| | 1 | 11280 | H3 01 | 96 | n/a | 744 | 711 |
| | 1 | 11281 | H3 02 | 97 | n/a | 745 | 711 |
| | 1 | 11282 | H3 03 | 98 | n/a | 746 | 711 |
| | 3 | 11283 | H3 04 | 99 | n/a | 747 | 711 |
| | 1 | 11284 | H3 05 | 100 | n/a | 748 | 711 |
| | 1 | 11285 | H3 06 | 101 | n/a | 749 | 711 |
| | 2 | 11286 | H3 07 | 102 | n/a | 750 | 711 |
| | 1 | 11287 | H3 08 | 103 | n/a | 751 | 711 |
| | 2 | 11288 | H3 09 | 104 | n/a | 752 | 711 |
| | 1 | 11289 | H3 10 | 105 | n/a | 753 | 711 |
| | 2 | 11290 | H3 11 | 106 | n/a | 754 | 711 |
| | 1 | 11291 | H3 12 | 107 | n/a | 755 | 711 |
| | 1 | 11292 | H3 13 | 108 | n/a | 756 | 711 |
| | 1 | 11293 | H3 14 | 109 | n/a | 757 | 711 |
| | 1 | 11294 | H3 15 | 110 | n/a | 758 | 711 |
| | 3 | 11295 | H3 16 | 111 | n/a | 759 | 711 |
| | 3 | 11296 | H3 17 | 112 | n/a | 760 | 711 |
| | 1 | 11297 | H3 18 | 113 | n/a | 761 | 711 |
| | 2 | 11298 | H3 19 | 114 | n/a | 762 | 711 |
| | 2 | 11299 | H3 20 | 115 | n/a | 763 | 711 |
| | 2 | 11300 | | | | 764 | 711 |
| A | 2 | 11301 | H1 03 | 28 | n/a | 765 | 711 |
| A | 3 | 11302 | H3 15 | 110 | n/a | 766 | 711 |
| A | 1 | 11303 | H3 19 | 114 | n/a | 767 | 711 |
| | 2 | 11304 | L1 01 | n/a | 24 | 710 | 768 |
| | 1 | 11305 | L1 02 | n/a | 25 | 710 | 769 |
| | 2 | 11306 | L1 03 | n/a | 26 | 710 | 770 |
| | 1 | 11307 | L1 04 | n/a | 27 | 710 | 771 |
| | 2 | 11308 | L1 05 | n/a | 28 | 710 | 772 |
| | 1 | 11309 | L1 06 | n/a | 29 | 710 | 773 |
| | 1 | 11310 | L1 07 | n/a | 30 | 710 | 774 |
| | 2 | 11311 | L1 08 | n/a | 31 | 710 | 775 |
| | 2 | 11312 | L1 09 | n/a | 32 | 710 | 776 |
| | 2 | 11313 | L1 10 | n/a | 33 | 710 | 777 |
| | 2 | 11314 | L1 11 | n/a | 34 | 710 | 778 |
| | 1 | 11315 | L1 12 | n/a | 35 | 710 | 779 |
| | 1 | 11316 | L1 13 | n/a | 36 | 710 | 780 |
| | 2 | 11317 | L2 01 | n/a | 52 | 710 | 781 |
| | 2 | 11318 | L2 02 | n/a | 53 | 710 | 782 |
| | 2 | 11319 | L2 03 | n/a | 54 | 710 | 783 |
| | 2 | 11320 | L2 04 | n/a | 55 | 710 | 784 |
| | 1 | 11321 | L2 05 | n/a | 56 | 710 | 785 |
| | 1 | 11322 | L2 06 | n/a | 57 | 710 | 786 |
| | 2 | 11323 | L2 07 | n/a | 58 | 710 | 787 |
| | 1 | 11324 | L3 01 | n/a | 91 | 710 | 788 |
| | 3 | 11325 | L3 02 | n/a | 92 | 710 | 789 |
| | 1 | 11326 | L3 03 | n/a | 93 | 710 | 790 |
| | 1 | 11327 | L3 04 | n/a | 94 | 710 | 791 |
| | 1 | 11328 | L3 05 | n/a | 95 | 710 | 792 |
| | 2 | 11329 | L3 06 | n/a | 96 | 710 | 793 |
| | 3 | 11330 | L3 07 | n/a | 97 | 710 | 794 |
| | 1 | 11331 | L3 08 | n/a | 98 | 710 | 795 |
| | 1 | 11332 | L3 09 | n/a | 99 | 710 | 796 |
| | 3 | 11333 | L3 10 | n/a | 100 | 710 | 797 |
| | 2 | 11334 | L3 11 | n/a | 101 | 710 | 798 |
| | 2 | 11335 | | | | 710 | 799 |
| A | 2 | 11336 | L1 09 | n/a | 32 | 710 | 800 |
| A | 1 | 11337 | L3 09 | n/a | 99 | 710 | 801 |
| A | 2 | 11338 | L3 10 | n/a | 100 | 710 | 802 |

TABLE 27
MYT9797 variants summary information, including SEQ ID NO
designations, amino acid substitution position information,
and binding data. Under “Binding Result”: 1 =
pH dependent binding; 2 = substantially similar binding;
and 3 = no binding, each relative to parent

| | | | CDR | HCV | LCV | VH | VL |
|---|---|---|---|---|---|---|---|
| Ala Sub Pos | Binding | MYT | Sub | Sub | Sub | SEQ | SEQ |
| (if present) | Result | # | Pos | Pos | Pos | ID | ID |
| | n/a | 9797 | n/a | n/a | n/a | 803 | 804 |
| | 2 | 11339 | H1 01 | 26 | n/a | 811 | 804 |
| | 2 | 11340 | H1 02 | 27 | n/a | 812 | 804 |
| | 2 | 11341 | H1 03 | 28 | n/a | 813 | 804 |
| | 2 | 11342 | H1 04 | 29 | n/a | 814 | 804 |
| | 2 | 11343 | H1 05 | 30 | n/a | 815 | 804 |
| | 2 | 11344 | H1 06 | 31 | n/a | 816 | 804 |
| | 2 | 11345 | H1 07 | 32 | n/a | 817 | 804 |
| | 1 | 11346 | H1 08 | 33 | n/a | 818 | 804 |
| | 2 | 11347 | H1 09 | 34 | n/a | 819 | 804 |
| | 2 | 11348 | H1 10 | 35 | n/a | 820 | 804 |
| | 1 | 11349 | H2 01 | 50 | n/a | 821 | 804 |
| | 2 | 11350 | H2 02 | 51 | n/a | 822 | 804 |
| | 3 | 11351 | H2 03 | 52 | n/a | 823 | 804 |
| | 1 | 11352 | H2 04 | 53 | n/a | 824 | 804 |
| | 3 | 11353 | H2 05 | 54 | n/a | 825 | 804 |
| | 1 | 11354 | H2 06 | 55 | n/a | 826 | 804 |
| | 1 | 11355 | H2 07 | 56 | n/a | 827 | 804 |
| | 1 | 11356 | H2 08 | 57 | n/a | 828 | 804 |
| | 1 | 11357 | H2 09 | 58 | n/a | 829 | 804 |
| | 2 | 11358 | H2 10 | 59 | n/a | 830 | 804 |
| | 2 | 11359 | H2 11 | 60 | n/a | 831 | 804 |
| | 2 | 11360 | H2 12 | 61 | n/a | 832 | 804 |
| | 2 | 11361 | H2 13 | 62 | n/a | 833 | 804 |
| | 2 | 11362 | H2 14 | 63 | n/a | 834 | 804 |
| | 1 | 11363 | H2 15 | 64 | n/a | 835 | 804 |
| | 2 | 11364 | H2 16 | 65 | n/a | 836 | 804 |
| | 3 | 11365 | H3 01 | 96 | n/a | 837 | 804 |
| | 2 | 11366 | H3 02 | 97 | n/a | 838 | 804 |
| | 3 | 11367 | H3 03 | 98 | n/a | 839 | 804 |
| | 2 | 11368 | H3 04 | 99 | n/a | 840 | 804 |
| | 1 | 11369 | H3 05 | 100 | n/a | 841 | 804 |
| | 3 | 11370 | H3 06 | 101 | n/a | 842 | 804 |
| | 3 | 11371 | H3 07 | 102 | n/a | 843 | 804 |
| | 3 | 11372 | H3 08 | 103 | n/a | 844 | 804 |
| | 3 | 11373 | H3 09 | 104 | n/a | 845 | 804 |
| | 3 | 11374 | H3 10 | 105 | n/a | 846 | 804 |
| | 2 | 11375 | H3 11 | 106 | n/a | 847 | 804 |
| | 1 | 11376 | H3 12 | 107 | n/a | 848 | 804 |
| | 2 | 11377 | H3 13 | 108 | n/a | 849 | 804 |
| | 1 | 11378 | | | n/a | 850 | 804 |
| A | 2 | 11379 | H1 03 | 28 | n/a | 851 | 804 |
| A | 1 | 11380 | H2 06 | 55 | n/a | 852 | 804 |
| A | 3 | 11381 | H3 06 | 101 | n/a | 853 | 804 |
| A | 3 | 11382 | H3 09 | 104 | n/a | 854 | 804 |
| | 2 | 11383 | L1 01 | n/a | 24 | 803 | 855 |
| | 3 | 11384 | L1 02 | n/a | 25 | 803 | 856 |
| | 2 | 11385 | L1 03 | n/a | 26 | 803 | 857 |
| | 2 | 11386 | L1 04 | n/a | 27 | 803 | 858 |
| | 2 | 11387 | L1 05 | n/a | 28 | 803 | 859 |
| | 3 | 11388 | L1 06 | n/a | 29 | 803 | 860 |
| | 3 | 11389 | L1 07 | n/a | 30 | 803 | 861 |
| | 1 | 11390 | L1 08 | n/a | 31 | 803 | 862 |
| | 2 | 11391 | L1 09 | n/a | 32 | 803 | 863 |
| | 1 | 11392 | L1 10 | n/a | 33 | 803 | 864 |
| | 1 | 11393 | L1 11 | n/a | 34 | 803 | 865 |
| | 1 | 11394 | L1 12 | n/a | 35 | 803 | 866 |
| | 3 | 11395 | L1 13 | n/a | 36 | 803 | 867 |
| | 1 | 11396 | L2 01 | n/a | 52 | 803 | 868 |
| | 3 | 11397 | L2 02 | n/a | 53 | 803 | 869 |
| | 1 | 11398 | L2 03 | n/a | 54 | 803 | 870 |
| | 2 | 11399 | L2 04 | n/a | 55 | 803 | 871 |
| | 2 | 11400 | L2 05 | n/a | 56 | 803 | 872 |
| | 2 | 11401 | L2 06 | n/a | 57 | 803 | 873 |
| | 2 | 11402 | L2 07 | n/a | 58 | 803 | 874 |
| | 2 | 11403 | L3 01 | n/a | 91 | 803 | 875 |
| | 3 | 11404 | L3 02 | n/a | 92 | 803 | 876 |
| | 3 | 11405 | L3 03 | n/a | 93 | 803 | 877 |
| | 3 | 11406 | L3 04 | n/a | 94 | 803 | 878 |
| | 3 | 11407 | L3 05 | n/a | 95 | 803 | 879 |
| | 2 | 11408 | L3 07 | n/a | 97 | 803 | 880 |
| | 1 | 11409 | L3 08 | n/a | 98 | 803 | 881 |
| | 1 | 11410 | L3 09 | n/a | 99 | 803 | 882 |
| | 3 | 11411 | L3 10 | n/a | 100 | 803 | 883 |
| | 3 | 11412 | L3 12 | n/a | 102 | 803 | 884 |
| | 2 | 11413 | L3 13 | n/a | 103 | 803 | 885 |
| | 2 | 11414 | | n/a | | 803 | 886 |
| | 3 | 11415 | | n/a | | 803 | 887 |
| | 3 | 11416 | | n/a | | 803 | 888 |
| A | 1 | 11417 | L1 11 | n/a | 34 | 803 | 889 |
| A | 1 | 11418 | L2 01 | n/a | 52 | 803 | 890 |
| A | 2 | 11419 | L3 03 | n/a | 93 | 803 | 891 |
| A | 2 | 11420 | L3 10 | n/a | 100 | 803 | 892 |

### Example 23. Characterization of Cellular Internalization and Endolysosomal Delivery of pH Engineered Anti-PTK7 ABPCs

Selected anti-PTK7 pH engineered antibody variants from Example 22 are analyzed for internalization and endolysosomal delivery PTK7+ cells (e.g. grown per providers instructions). Cell counts are determined using trypan blue staining and the Countess II FL Automated Cell Counter (ThermoFisher; AMQAF1000). Cells are then diluted to 75,000 cells/mL and 100 μl/well are seeded into 96-well flat bottom cell culture plates (Genesee Scientific; 25-109) for overnight incubation at 37° C. and 5% CO₂. The following day, anti-PTK7 pH engineered antibody variants, starting ABPC antibodies, control IgG1 isotype control (BP0297, Bioxcell), and vehicle control are diluted in native culture media, and then mixed 1:1 with a 3× molar ratio Zenon pHrodo iFL Human IgG Labeling Reagent (ThermoFisher; Z25611). The mixture is incubated for 30 minutes at room temperature, the existing media is removed from the plated cells and the mixture is added. The mixture of cells, anti-PTK7 antibody variants, and Zenon pHrodo iFL Human IgG Labeling Reagent was incubated at 37° C., 5% CO₂ for 24 hours. Following incubation, the media is removed and cells are washed with 100 μL of room temperature culture medium. Following the wash 50 μL of Trypsin solution (ThermoFisher Scientific; 12605036) is added and the plate incubated at 37° C. until most of the cells are detached, followed by addition of 50 μL of culture medium. Cells are transferred to a 96-well U-bottom plate (Genesee Scientific; 25-221) and spun down at 2000 RPM for 2 minutes. Cells are washed 2× with 200 μL of ice-cold FACS buffer (phosphate buffered saline (PBS), pH 7.4+2 mM ethylenediaminetetraacetic acid (EDTA)+2% (v/v) HI FBS) before being resuspended in 150 μL of FACS buffer. Cells (Median green fluorescence intensity is detected using an Attune NxT flow cytometer (ThermoFisher Scientific). Data is analyzed using Flowjo analysis software. pHrodo green is a pH sensitive dye that fluoresces in the low pH environment of the endosomes and lysosomes and therefore can be used to quantify antibody internalization and endolysosomal delivery. Internalization and endolysosomal delivery of anti-PTK7 starting ABPC's and variants at 25 nM in PTK7+ cells is measured by pHrodo green median fluorescence intensity. Several pH engineered anti-PTK7 antibody variants show increased median fluorescence intensity relative to their corresponding starting ABPC antibodies demonstrating that increased dissociation at lower pH leads to enhanced internalization and endolysosomal delivery inside cells as shown by increased fluorescence or increased fluorescence as compared to IgG1 isotype control. Increased endolysosomal delivery is quantitated for each pH engineered anti-PTK7 antibody variant on the top of each bar as a ratio of: the variant's median fluorescence intensity minus the median fluorescence intensity of the IgG control, then all divided by the variant's corresponding starting ABPC's median fluorescence intensity minus the median fluorescence intensity of the IgG control. For example, pH-engineered variant antibodies as disclosed herein may show increased internalization and endolysosomal delivery relative to their respective parental antibodies, as particularly as exemplified by the following: MYT9345 variants (MYT10899, MYT10917 and MYT10939); MYT9359 variants (MYT10952, MYT10955, MYT10989, MYT11005, MYT11017 and MYT11021); MYT9361 variants (MYT11053, MYT11056, MYT11058-59, MYT11063, MYT11068-69, MYT11078-79, MYT11081, MYT11084, MYT11096, and MYT11100; MYT9412 variants (MYT11142-43); MYT9460 variants (MYT11190, MYT11211-12, and MYT11232), MYT9792 variants (MYT11282, MYT11284, MYT11287, MYT11291-94, MYT11297, MYT11303, MYT11305, MYT11307, MYT11310, MYT11315-16, MYT11321, MYT11324, MYT11326-28, MYT11331-32, and MYT11337); and or MYT9797 variant (MYT11346).

Such pH engineered anti-PTK7 antibody variants with increased median fluorescence intensity relative to their starting ABPC antibodies are selected for further analysis.

TABLE 28
Internalization results for exemplary PTK7-binding proteins.
FOW = fold over the wildtype, parental antibody
(with a primary antibody concentration @ 25 nM)

| MYT Number | HCVD SEQ ID | LCVD SEQ ID | FOW @ 25 nM |
|---|---|---|---|
| 10899 | 323 | 302 | 1.14 |
| 10917 | 341 | 302 | 1.29 |
| 10952 | 384 | 376 | 1.06 |
| 10955 | 387 | 376 | 1.05 |
| 10989 | 421 | 376 | 1.09 |
| 11005 | 375 | 437 | 1.05 |
| 11017 | 375 | 449 | 1.20 |
| 11033 | 473 | 460 | 1.19 |
| 11053 | 493 | 460 | 1.25 |
| 11056 | 496 | 460 | 1.10 |
| 11058 | 498 | 460 | 1.26 |
| 11059 | 499 | 460 | 1.27 |
| 11063 | 503 | 460 | 2.04 |
| 11068 | 508 | 460 | 1.22 |
| 11069 | 509 | 460 | 1.22 |
| 11078 | 459 | 518 | 1.24 |
| 11079 | 459 | 519 | 1.20 |
| 11081 | 459 | 521 | 1.22 |
| 11084 | 459 | 524 | 1.14 |
| 11096 | 459 | 536 | 1.43 |
| 11100 | 459 | 540 | 1.17 |
| 11142 | 590 | 543 | 1.29 |
| 11143 | 591 | 543 | 1.61 |
| 11190 | 646 | 629 | 1.32 |
| 11211 | 667 | 629 | 1.14 |
| 11212 | 668 | 629 | 1.23 |
| 11232 | 628 | 688 | 1.15 |
| 11257 | 721 | 711 | 1.11 |
| 11259 | 723 | 711 | 1.14 |
| 11261 | 725 | 711 | 1.24 |
| 11264 | 728 | 711 | 1.13 |
| 11271 | 735 | 711 | 1.57 |
| 11280 | 744 | 711 | 1.16 |
| 11281 | 745 | 711 | 1.11 |
| 11282 | 746 | 711 | 1.68 |
| 11284 | 748 | 711 | 1.51 |
| 11287 | 751 | 711 | 1.71 |
| 11291 | 755 | 711 | 1.53 |
| 11292 | 756 | 711 | 1.59 |
| 11293 | 757 | 711 | 1.65 |
| 11294 | 758 | 711 | 1.66 |
| 11297 | 761 | 711 | 1.51 |
| 11303 | 767 | 711 | 1.51 |
| 11305 | 710 | 769 | 1.37 |
| 11307 | 710 | 771 | 1.82 |
| 11310 | 710 | 774 | 1.64 |
| 11315 | 710 | 779 | 1.51 |
| 11316 | 710 | 780 | 1.55 |
| 11321 | 710 | 785 | 1.36 |
| 11324 | 710 | 788 | 1.58 |
| 11326 | 710 | 790 | 1.29 |
| 11327 | 710 | 791 | 1.60 |
| 11328 | 710 | 792 | 1.99 |
| 11331 | 710 | 795 | 1.77 |
| 11332 | 710 | 796 | 1.77 |
| 11337 | 710 | 801 | 1.68 |
| 11346 | 818 | 804 | 1.16 |
| 11418 | 803 | 890 | 1.19 |

TABLE 29
Internalization results for exemplary PTK7-binding proteins.
FOW = fold over the wildtype, parental antibody
(with a primary antibody concentration @ 25 nM)

| MYT | FOW | MYT | FOW |
|---|---|---|---|
| MYT6158 | 1.12 | MYT7816 | 1.34 |
| MYT7843 | 1.15 | MYT7817 | 1.38 |
| MYT7845 | 1.30 | MYT7818 | 1.43 |
| MYT7853 | 1.29 | MYT7819 | 1.45 |
| MYT7854 | 1.12 | MYT7820 | 1.16 |
| MYT6238 | 1.28 | MYT7822 | 1.36 |
| MYT6241 | 1.26 | MYT7824 | 1.18 |
| MYT6260 | 1.46 | MYT7827 | 2.18 |
| MYT6238 | 1.27 | MYT7828 | 1.14 |
| MYT6241 | 1.23 | MYT7829 | 1.60 |
| MYT6257 | 1.20 | MYT7830 | 1.4 |
| MYT6275 | 1.14 | MYT7999 | 1.16 |
| MYT6290 | 1.87 | MYT8001 | 1.12 |
| MYT7836 | 1.23 | MYT8004 | 1.11 |
| MYT7974 | 1.11 | MYT8015 | 1.10 |
| MYT7998 | 1.08 | MYT6260 | 1.15 |
| | | MYT6290 | 1.51 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A pharmaceutical composition comprising an effective amount of an antigen-binding protein construct (ABPC) comprising: a first antigen-binding domain (ABD) that is capable of specifically binding PTK7 or an epitope of PTK7 presented on the surface of a target mammalian cell, wherein: the dissociation rate of the first ABD at a pH of ~4.0-6.5 is faster than the dissociation rate at a pH of ~7.0-8.0; and/or the KD of the first ABD at a pH of ~4.0-6.5 is greater than the K_{D} at a pH of ~7.0-8.0; and optionally wherein the ABPC exhibits increased target-specific uptake by cancer cells expressing PTK7 relative to the uptake by the same cells of a suitable control ABPC.

2. The composition of claim 1, wherein the first ABD comprises a heavy chain variable domain (HCVD) of MYT9345, MYT9359, MYT9361, MYT9412, MYT9460, MYT9792, or MYT9797, optionally with one or more histidine substitution; or an HCVD of cofetuzumab, 7C8, or 12C6, each with at least one histidine substitution.

3. The composition of claim 1, wherein the first ABD comprises a light chain variable domain (LCVD) of MYT9345, MYT9359, MYT9361, MYT9412, MYT9460, MYT9792, or MYT9797, optionally with one or more histidine substitution; or an LCVD of cofetuzumab, 7C8, or 12C6, each with at least one histidine substitution.

4. The composition of any one of claim 1, wherein the ABD comprises one of: (a) an HCVD of MYT9345 and/or an LCVD of MYT9345; (b) an HCVD of MYT9359 and/or an LCVD of MYT9359; (c) an HCVD of MYT9361; and/or an LCVD of MYT9361; (d) an HCVD of MYT9412 and/or an LCVD of MYT9412; (e) an HCVD of MYT9460 and/or an LCVD of MYT9460; (f) an HCVD of MYT9792; and/or an LCVD of MYT9792; and (g) an HCVD of MYT9797 and/or an LCVD of MYT9797; wherein each HCVD and/or LCVD optionally has one or more histidine substitution; or, the ABD comprises one of: (h) an HCVD of cofetuzumab and/or an LCVD of cofetuzumab; (i) an HCVD of 7C8 and/or an LCVD of 7C8; and (j) an HCVD of 12C6; and/or an LCVD of 12C6, each HCVD and/or LCVD of (i), (h), or (j) having at least one histidine substitution.

5. The composition of any one of claims 1 to 4, wherein the HCVD comprises one of the sequence as set forth in SEQ ID NOs: 1, 126, 229, 301, 375, 459, 542, 628, 710, and 803.

6. The composition of any one of claims 1 to 4, wherein the LCVD comprises one of the sequences as set forth in SEQ ID NOs: 2, 127, 230, 302, 376, 460, 543, 629, 711, and 804.

7. The composition of claim 1, wherein the first ABD comprises an HCVD comprising the CDR1, 2, and 3 of one of: (a) SEQ ID NOs: 303, 304, and 305; (b) SEQ ID NOs: 377, 378, and 379; (c) SEQ ID NOs: 461, 462, and 463; (d) SEQ ID NOs: 544, 545, and 546, (e) SEQ ID NOs: 630, 631, and 632; (f) SEQ ID NOs: 712, 713, and 714; (g) SEQ ID NOs: 805, 806, and 807, (h) SEQ ID NOs: 3, 4, and 5; (i) SEQ ID NOs: 128, 129, and 130; and (j) SEQ ID NOs: 231, 232, and 233, wherein for (a) to (g), each set of three CDRs optionally has collectively one or more position substituted with a histidine, and for (h) to (j), each set of three CDRs has collectively at least one or more position substituted with a histidine.

8. The composition of claim 1, wherein the first ABD comprises an LCVD comprising the CDR1, 2, and 3 of one of: (a) SEQ ID NOs: 306, 307, and 308; (b) SEQ ID NOs: 380, 381, and 382; (c) SEQ ID NOs: 464, 465, and 466; (d) SEQ ID NOs: 547, 548, and 549, (e) SEQ ID NOs: 633, 634, and 635; (f) SEQ ID NOs: 715, 716, and 717; (g) SEQ ID NOs: 808, 809, and 810, (h) SEQ ID NOs: 6, 7, and 8; (i) SEQ ID NOs: 131, 132, and 133; and (j) SEQ ID NOs: 234, 235, and 236;
wherein for (a) to (g), each set of three CDRs optionally has collectively one or more position substituted with a histidine, and for (h) to (j), each set of three CDRs has collectively at least one or more position substituted with a histidine.

9. The composition of claim 1, wherein the first ABD comprises one of:
(a) an HCVD comprising the CDR1, 2, and 3 of SEQ ID NOs: 303, 304, and 305; and/or an LCVD comprising the CDR1, 2, and 3 of one of SEQ ID NOs: 306, 307, and 308;
(b) an HCVD comprising the CDR1, 2, and 3 of one of SEQ ID NOs: 377, 378, and 379; and/or an LCVD comprising the CDR1, 2, and 3 of SEQ ID NOs: 380, 381, and 382;
(c) an HCVD comprising the CDR1, 2, and 3 of SEQ ID NOs: 461, 462, and 463; and/or an LCVD comprising the CDR1, 2, and 3 of SEQ ID NOs: 464, 465, and 466;
(d) an HCVD comprising the CDR1, 2, and 3 of SEQ ID NOs: 544, 545, and 546; and/or an LCVD comprising the CDR1, 2, and 3 of SEQ ID NOs: 547, 548, and 549,
(e) an HCVD comprising the CDR1, 2, and 3 of one of SEQ ID NOs: 630, 631, and 632; and/or an LCVD comprising the CDR1, 2, and 3 of SEQ ID NOs: 633, 634, and 635;
(f) an HCVD comprising the CDR1, 2, and 3 of SEQ ID NOs: 712, 713, and 714; and/or an LCVD comprising the CDR1, 2, and 3 of SEQ ID NOs: 715, 716, and 717;
(g) an HCVD comprising the CDR1, 2, and 3 of SEQ ID NOs: 805, 806, and 807; and/or an LCVD comprising the CDR1, 2, and 3 of SEQ ID NOs: 808, 809, and 810;
(h) an HCVD comprising the CDR1, 2, and 3 of one of SEQ ID NOs: 3, 4, and 5; and/or an LCVD comprising the CDR1, 2, and 3 of SEQ ID NOs: 6, 7, and 8;
(i) an HCVD comprising the CDR1, 2, and 3 of SEQ ID NOs: 128, 129, and 130; and/or an LCVD comprising the CDR1, 2, and 3 of SEQ ID NOs: 131, 132, and 133; and
(j) an HCVD comprising the CDR1, 2, and 3 of SEQ ID NOs: 231, 232, and 233; and/or an LCVD comprising the CDR1, 2, and 3 of SEQ ID NOs: 234, 235, and 236;
wherein for (a) to (g), either or both of each set of three CDRs optionally has collectively one or more position substituted with a histidine, and
wherein for (h) to (j), either or both of each set of three CDRs has collectively at least one or more position substituted with a histidine.

10. The composition of claim 1, 2, or 7, wherein the first ABD comprises one of:
(a) an HCVD that is at least 90% identical to SEQ ID NO: 301, wherein the HCVD optionally includes a histidine at one or more positions in SEQ ID NO: 301 selected from: 29, 30, 32, 50-54, 58, 60, 96, and 101;
(b) an HCVD that is at least 90% identical to SEQ ID NO: 375, wherein the HCVD optionally includes a histidine at one or more positions in SEQ ID NO: 375 selected from: 26, 27, 30, 32, 34, 51, 52, 55, 58, 59, 63, 97, 99, and 108;
(c) an HCVD that is at least 90% identical to SEQ ID NO: 459, wherein the HCVD optionally includes a histidine at one or more positions in SEQ ID NO: 459 selected from: 32, 99, 101, 102, 106 and 111;
(d) an HCVD that is at least 90% identical to SEQ ID NO: 542, wherein the HCVD optionally includes a histidine at one or more positions in SEQ ID NO: 542 selected from: 27, 29, 30, 31, 32, 53, 55, 58, 102, 104 and 105;
(e) an HCVD that is at least 90% identical to SEQ ID NO: 628, wherein the HCVD optionally includes a histidine at one or more positions in SEQ ID NO: 628 selected from: 26, 28, 30, 50, 52, 53, 54, 55, 56, 57, 100, 101, 102 and 103;
(f) an HCVD that is at least 90% identical to SEQ ID NO: 710, wherein the HCVD optionally includes a histidine at one or more positions in SEQ ID NO: 710 selected from: 29, 30, 31, 33, 35, 50, 53, 57, 58, 65, 96, 97, 98, 100, 101, 103, 105, 107, 108, 109, 110 and 113;
(g) an HCVD that is at least 90% identical to SEQ ID NO: 803, wherein the HCVD optionally includes a histidine at one or more positions in SEQ ID NO: 803 selected from: 33, 50, 53, 55-58, 64, 100, and 107;
(h) an HCVD that is at least 90% identical to SEQ ID NO: 1, wherein the HCVD includes a histidine at one or more positions in SEQ ID NO: 1 selected from the group consisting of: 24, 27, 28, 29, 30, 31, 32, 34, 53, 54, 55, 57, 58, 64, 98, 100, 102, 103, and 107;
(i) an HCVD that is at least 90% identical to SEQ ID NO: 126, wherein the HCVD includes a histidine at one or more positions in SEQ ID NO: 126 selected from the group consisting of: 53, 54, 56, 57, 60, 102, 103, 104, 105, 106, 108, 109, 110, and 111; and
(j) an HCVD that is at least 90% identical to SEQ ID NO: 229, wherein the HCVD includes a histidine at one or more positions in SEQ ID NO: 229 selected from the group consisting of: 27, 32, 33, 34, 35, 51, 54, 56, 58, and 100.

11. The composition of claim 1, 2, or 7, wherein the first ABD comprises one of:
(a) an LCVD that is at least 90% identical to SEQ ID NO: 302, wherein the LCVD optionally includes a histidine at one or more positions in SEQ ID NO: 302 selected from: 25, 30-33, 35, 55-57, 91, 94-96, and 98;
(b) an LCVD that is at least 90% identical to SEQ ID NO: 376, wherein the LCVD optionally includes a histidine at one or more positions in SEQ ID NO: 376 selected from: 25, 29, 30, 31, 32, 33, 50, 51, 52, 53, 89, 91, 92, 93, 96, 97, 100 and 102;
(c) an LCVD that is at least 90% identical to SEQ ID NO: 460, wherein the LCVD optionally includes a histidine at one or more positions in SEQ ID NO: 460 selected from: 30, 31, 33, 51, 92 and 95;
(d) an LCVD that is at least 90% identical to SEQ ID NO: 543, wherein the LCVD optionally includes a histidine at one or more positions in SEQ ID NO: 543 selected from: 25, 29, 30, 31, 32, 89, 92 and 98;
(e) an LCVD that is at least 90% identical to SEQ ID NO: 629, wherein the LCVD optionally includes a histidine at one or more positions in SEQ ID NO: 629 selected from: 25,
(f) an LCVD that is at least 90% identical to SEQ ID NO: 711, wherein the LCVD optionally includes a histidine at one or more positions in SEQ ID NO: 711 selected from: 25, 27, 29, 30, 35, 36, 56, 57, 91, 93, 94, 95, 98 and 99;
(g) an LCVD that is at least 90% identical to SEQ ID NO: 804, wherein the LCVD optionally includes a histidine at one or more positions in SEQ ID NO: 804 selected from: 31, 33-35, 52, 54, 98, and 99;
(h) an LCVD that is at least 90% identical to SEQ ID NO: 2, wherein the LCVD includes a histidine at one or more positions in SEQ ID NO: 2 selected from the group consisting of: 25, 29, 31, 35, 60, 93, and 94;
(i) an LCVD that is at least 90% identical to SEQ ID NO: 127, wherein the LCVD includes a histidine at one or more positions in SEQ ID NO: 127 selected from the group consisting of: 25, 26, 28, 29, 31, 32, 33, 50, 90, 92, and 94; and
(j) an LCVD that is at least 90% identical to SEQ ID NO: 230, wherein the LCVD includes a histidine at one or more positions in SEQ ID NO: 230 selected from the group consisting of: 30, 31, 33, 51, 53, 57, 91, 92, 94, and 99.

12. The composition of claim 1, 2, or 7, wherein the first ABD comprises:
(a) an HCVD that is at least 90% identical to SEQ ID NO: 301, wherein the HCVD optionally includes a histidine at two or more positions in SEQ ID NO: 301 selected from: 29, 30, 32, 50-54, 58, 60, 96, and 101;
(b) an HCVD that is at least 90% identical to SEQ ID NO: 375, wherein the HCVD optionally includes a histidine at two or more positions in SEQ ID NO: 375 selected from: 26, 27, 30, 32, 34, 51, 52, 55, 58, 59, 63, 97, 99, and 108;
(c) an HCVD that is at least 90% identical to SEQ ID NO: 459, wherein the HCVD optionally includes a histidine at two or more positions in SEQ ID NO: 459 selected from: 32, 99, 101, 102, 106 and 111;
(d) an HCVD that is at least 90% identical to SEQ ID NO: 542, wherein the HCVD optionally includes a histidine at two or more positions in SEQ ID NO: 542 selected from: 27, 29, 30, 31, 32, 53, 55, 58, 102, 104 and 105;
(e) an HCVD that is at least 90% identical to SEQ ID NO: 628, wherein the HCVD optionally includes a histidine at two or more positions in SEQ ID NO: 628 selected from: 26, 28, 30, 50, 52, 53, 54, 55, 56, 57, 100, 101, 102 and 103;
(f) an HCVD that is at least 90% identical to SEQ ID NO: 710, wherein the HCVD optionally includes a histidine at two or more positions in SEQ ID NO: 710 selected from: 29, 30, 31, 33, 35, 50, 53, 57, 58, 65, 96, 97, 98, 100, 101, 103, 105, 107, 108, 109, 110 and 113;
(g) an HCVD that is at least 90% identical to SEQ ID NO: 803, wherein the HCVD optionally includes a histidine at two or more positions in SEQ ID NO: 803 selected from: 33, 50, 53, 55-58, 64, 100, and 107;
(h) an HCVD that is at least 90% identical to SEQ ID NO: 1, wherein the HCVD includes a histidine at two or more positions in SEQ ID NO: 1 selected from the group consisting of: 24, 27, 28, 29, 30, 31, 32, 34, 53, 54, 55, 57, 58, 64, 98, 100, 102, 103, and 107;
(i) an HCVD that is at least 90% identical to SEQ ID NO: 126, wherein the HCVD includes a histidine at two or more positions in SEQ ID NO: 126 selected from the group consisting of: 53, 54, 56, 57, 60, 102, 103, 104, 105, 106, 108, 109, 110, and 111; and
(j) an HCVD that is at least 90% identical to SEQ ID NO: 229, wherein the HCVD includes a histidine at two or more positions in SEQ ID NO: 229 selected from the group consisting of: 27, 32, 33, 34, 35, 51, 54, 56, 58, and 100.

13. The composition of claim 1, 3, or 8, wherein the first ABD comprises one of:
(a) an LCVD that is at least 90% identical to SEQ ID NO: 302, wherein the LCVD optionally includes a histidine at two or more positions in SEQ ID NO: 302 selected from: 25, 30-33, 35, 55-57, 91, 94-96, and 98;
(b) an LCVD that is at least 90% identical to SEQ ID NO: 376, wherein the LCVD optionally includes a histidine at two or more positions in SEQ ID NO: 376 selected from: 25, 29, 30, 31, 32, 33, 50, 51, 52, 53, 89, 91, 92, 93, 96, 97, 100 and 102;
(c) an LCVD that is at least 90% identical to SEQ ID NO: 460, wherein the LCVD optionally includes a histidine at two or more positions in SEQ ID NO: 460 selected from: 30, 31, 33, 51, 92 and 95;
(d) an LCVD that is at least 90% identical to SEQ ID NO: 543, wherein the LCVD optionally includes a histidine at two or more positions in SEQ ID NO: 543 selected from: 25, 29, 30, 31, 32, 89, 92 and 98;
(e) an LCVD that is at least 90% identical to SEQ ID NO: 629, wherein the LCVD optionally includes a histidine at two or more positions in SEQ ID NO: 629 selected from: 25,
(f) an LCVD that is at least 90% identical to SEQ ID NO: 711, wherein the LCVD optionally includes a histidine at two or more positions in SEQ ID NO: 711 selected from: 25, 27, 29, 30, 35, 36, 56, 57, 91, 93, 94, 95, 98 and 99;
(g) an LCVD that is at least 90% identical to SEQ ID NO: 804, wherein the LCVD optionally includes a histidine at two or more positions in SEQ ID NO: 804 selected from: 31, 33-35, 52, 54, 98, and 99;
(h) an LCVD that is at least 90% identical to SEQ ID NO: 2, wherein the LCVD includes a histidine at two or more positions in SEQ ID NO: 2 selected from the group consisting of: 25, 29, 31, 35, 60, 93, and 94;
(i) an LCVD that is at least 90% identical to SEQ ID NO: 127, wherein the LCVD includes a histidine at two or more positions in SEQ ID NO: 127 selected from the group consisting of: 25, 26, 28, 29, 31, 32, 33, 50, 90, 92, and 94; and
(j) an LCVD that is at least 90% identical to SEQ ID NO: 230, wherein the LCVD includes a histidine at two or more positions in SEQ ID NO: 230 selected from the group consisting of: 30, 31, 33, 51, 53, 57, 91, 92, 94, and 99.

14. The composition of claim 1, 4, or 9, wherein the first ABD comprises one of:
(a) an HCVD that is at least 90% identical to SEQ ID NO: 301, wherein the HCVD optionally includes a histidine at one or more positions in SEQ ID NO: 301 selected from 29, 30, 32, 50-54, 58, 60, 96, and 101; and/or an LCVD that is at least 90% identical to SEQ ID NO: 302, wherein the LCVD optionally includes a histidine at one or more positions in SEQ ID NO: 302 selected from 25, 30-33, 35, 55-57, 91, 94-96, and 98;
(b) an HCVD that is at least 90% identical to SEQ ID NO: 375, wherein the HCVD optionally includes a histidine at one or more positions in SEQ ID NO: 375 selected from 26, 27, 30, 32, 34, 51, 52, 55, 58, 59, 63, 97, 99, and 108; and/or an LCVD that is at least 90% identical to SEQ ID NO: 376, wherein the LCVD optionally includes a histidine at one or more positions in SEQ ID NO: 376 selected from 25, 29, 30, 31, 32, 33, 50, 51, 52, 53, 89, 91, 92, 93, 96, 97, 100 and 102;
(c) an HCVD that is at least 90% identical to SEQ ID NO: 459, wherein the HCVD optionally includes a histidine at one or more positions in SEQ ID NO: 459 selected from 32, 99, 101, 102, 106 and 111; and/or an LCVD that is at least 90% identical to SEQ ID NO: 460, wherein the LCVD optionally includes a histidine at one or more positions in SEQ ID NO: 460 selected from 30, 31, 33, 51, 92 and 95;
(d) an HCVD that is at least 90% identical to SEQ ID NO: 542, wherein the HCVD optionally includes a histidine at one or more positions in SEQ ID NO: 542 selected from 27, 29, 30, 31, 32, 53, 55, 58, 102, 104 and 105; and/or an LCVD that is at least 90% identical to SEQ ID NO: 543, wherein the LCVD optionally includes a histidine at one or more positions in SEQ ID NO: 543 selected from 25, 29, 30, 31, 32, 89, 92 and 98;
(e) an HCVD that is at least 90% identical to SEQ ID NO: 628, wherein the HCVD optionally includes a histidine at one or more positions in SEQ ID NO: 628 selected from 26, 28, 30, 50, 52, 53, 54, 55, 56, 57, 100, 101, 102 and 103; and/or an LCVD that is at least 90% identical to SEQ ID NO: 629, wherein the LCVD optionally includes a histidine at one or more positions in SEQ ID NO: 629 selected from 25, 26, 28, 29, 33, 51, 55, 89, 91, 95, 96, 97, 98, 99 and 100;
(f) an HCVD that is at least 90% identical to SEQ ID NO: 710, wherein the HCVD optionally includes a histidine at one or more positions in SEQ ID NO: 710 selected from 29, 30, 31, 33, 35, 50, 53, 57, 58, 65, 96, 97, 98, 100, 101, 103, 105, 107, 108, 109, 110 and 113; and/or an LCVD that is at least 90% identical to SEQ ID NO: 711, wherein the LCVD optionally includes a histidine at one or more positions in SEQ ID NO: 711 selected from 25, 27, 29, 30, 35, 36, 56, 57, 91, 93, 94, 95, 98 and 99;
(g) an HCVD that is at least 90% identical to SEQ ID NO: 803, wherein the HCVD optionally includes a histidine at one or more positions in SEQ ID NO: 803 selected from 33, 50, 53, 55-58, 64, 100, and 107; and/or an LCVD that is at least 90% identical to SEQ ID NO: 804, wherein the LCVD optionally includes a histidine at one or more positions in SEQ ID NO: 804 selected from 31, 33-35, 52, 54, 98, and 99.

15. The composition of claim 1, wherein the first ABD comprises an HCVD comprising the sequence of one of SEQ ID NO: 301, or one of 309-342; SEQ ID NO: 375, or one of 383-423; SEQ ID NO: 459, or one of 467-511; SEQ ID NO: 542, or one of 550-592; SEQ ID NO: 628, or one of 636-675; SEQ ID NO: 710, or one of 718-767; and SEQ ID NO: 803, or one of 811-854; or wherein the HCVD comprises one of the following: (a) an HCVD of SEQ ID NO: 1, 14, 17, 18, 19, 20, 21, 22, 24, 25, 29, 30, 31, 33, 34, 40, 44, 46, 48, 49, 53, 86, 87, 88, 89, 90, 91, 147, 148, 150, 151, 154, 166, 167, 168, 169, 170, 172, 173, 174, 175, 208, 209, 210, 211, 212, 213, 214, 215, 216, 217, 218, 219, 220, 221, 222, 224, 225, 226, or 227; and (c) an HCVD of SEQ ID NO: 229, 238, 243, 244, 245, 246, 248, 251, 253, 255, or 267.

16. The composition of claim 1 or 15, wherein the first ABD comprises an LCVD comprising the sequence of one of SEQ ID NO: 302, or one of 343-374; SEQ ID NO: 376, or one of 424-458; SEQ ID NO: 460, or one of 512-541; SEQ ID NO: 543, or one of 593-627; SEQ ID NO: 629, or one of 676-709; SEQ ID NO: 711, or one of 768-854; and SEQ ID NO: 804, or one of 855-892; or wherein the LCVD comprises one of the following: (a) an LCVD of SEQ ID NO: 2, 56, 60, 62, 66, 69, 76, 77, 78, 110, 113, 114, 119, 122, 123, 124, or 125; (b) an LCVD of SEQ ID NO: 127, 181, 182, 184, 185, 187, 188, 189, 191, 199, 201, 203, or 228; and (c) an LCVD of SEQ ID NO: 230, 275, 276, 278, 281, 283, 287, 289, 290, 292, or 297.

17. The composition of claim 1, wherein the first ABD comprises one of:
(a) an HCVD of one of SEQ ID NO: 301, or one of 309-342, and/or an LCVD of one of SEQ ID NO: 302, or one of 343-374, wherein the first ABD does not comprise (i) SEQ ID NOs: 301 and 5; (ii) an HCVD of SEQ ID NO: 301 and an LCVD that is not one of SEQ ID NOs: 344, 349-352, 354, 359-361, 363, 366-368, 370, and 373; or (iii) an LCVD of SEQ ID NO: 302 and an HCVD that is not one of SEQ ID NO: 312, 313, 315, 319-323, 327, 329, 335, 340 and 341;
(b) an HCVD of one of SEQ ID NO: 375, or one of 383-423, and/or an LCVD of one of SEQ ID NO: 376, or one of 424-458, wherein the first ABD does not comprise (i) SEQ ID NOs: 375 and 79; (ii) an HCVD of SEQ ID NO: 375 and an LCVD that is not one of SEQ ID NOs: 425, 429, 430, 431, 432, 433, 435, 436, 437, 438, 442, 444, 445, 446, 449, 450, 453, and 455; or (iii) an LCVD of SEQ ID NO: 376 and an HCVD that is not one of SEQ ID NO: 383, 384, 387, 389, 391, 394, 395, 398, 401, 402, 406, 410, 412, and 421;
(c) an HCVD of one of SEQ ID NO: 459, or one of 467-511, and/or an LCVD of one of SEQ ID NO: 460, or one of 512-541, wherein the first ABD does not comprise (i) SEQ ID NOs: 459 and 163; (ii) an HCVD of SEQ ID NO: 459 and an LCVD that is not one of SEQ ID NOs: 518, 519, 521, 524, 533, and 536; or (iii) an LCVD of SEQ ID NO: 460 and an HCVD that is not one of SEQ ID NO: 473, 496, 498, 499, 503, and 508;
(d) an HCVD of one of SEQ ID NO: 542, or one of 550-592, and/or an LCVD of one of SEQ ID NO: 543, or one of 593-627, wherein the first ABD does not comprise (i) SEQ ID NOs: 542 and 246; (ii) an HCVD of SEQ ID NO: 542 and an LCVD that is not one of SEQ ID NOs: 594, 598, 599, 600, 601, 611, 614, and 620; or (iii) an LCVD of SEQ ID NO: 543 and an HCVD that is not one of SEQ ID NO: 551, 553, 554, 555, 556, 563, 565, 568, 582, 584, and 585;
(e) an HCVD of one of SEQ ID NO: 628, or one of 636-675, and/or an LCVD of one of SEQ ID NO: 629, or one of 676-709, wherein the first ABD does not comprise (i) SEQ ID NOs: 628 and 332; (ii) an HCVD of SEQ ID NO: 628 and an LCVD that is not one of SEQ ID NOs: 677, 678, 680, 681, 685, 688, 692, 694, 696, 700, 701, 702, 703, 704, and 705; or (iii) an LCVD of SEQ ID NO: 629 and an HCVD that is not one of SEQ ID NO: 636, 638, 640, 646, 648, 649, 650, 651, 652, 653, 666, 667, 668, and 669;
(f) an HCVD of one of SEQ ID NO: 710, or one of 718-767, and/or an LCVD of one of SEQ ID NO: 711, or one of 768-802, wherein the first ABD does not comprise (i) SEQ ID NOs: 710 and 414; (ii) an HCVD of SEQ ID NO: 710 and an LCVD that is not one of SEQ ID NOs: 769, 771, 773, 774, 779, 780, 785, 786, 788, 790, 791, 792, 795, and 796; or (iii) an LCVD of SEQ ID NO: 711 and an HCVD that is not one of SEQ ID NO: 721, 722, 723, 725, 727, 728, 731, 735, 736, 743, 744, 745, 746, 748, 749, 751, 753, 755, 756, 757, 758, and 761;
(g) an HCVD of one of SEQ ID NO: 803, or one of 811-854, and/or an LCVD of one of SEQ ID NO: 804, or one of 855-892, wherein the first ABD does not comprise (i) SEQ ID NOs: 803 and 507; (ii) an HCVD of SEQ ID NO: 803 and an LCVD that is not one of SEQ ID NOs: 862, 864-866, 868, 870, 881, and 882; or (iii) an LCVD of SEQ ID NO: 804 and an HCVD that is not one of SEQ ID NO: 818, 821, 824, 826-829, 835, 841, 848, and 850;
(h) an HCVD of SEQ ID NO: 14, 17, 18, 19, 20, 21, 22, 24, 25, 29, 30, 31, 33, 34, 40, 44, 46, 48, 49, 53, 86, 87, 88, 89, 90, 91, 92, 93, 94, 95, 96, 98, 99, 100, 101, 102, 103, 104, or 105, and/or an LCVD of SEQ ID NO: 2, 56, 60, 62, 66, 69, 76, 77, 78, 110, 113, 114, 119, 122, 123, 124, or 125, wherein the first antigen binding domain does not comprise (i) an HCVD of SEQ ID NO: 1 and an LCVD of SEQ ID NO: 2; (ii) an HCVD of SEQ ID NO: 1 and LCVD that is not one of SEQ ID NOs: SEQ ID NOs: 56, 60, 62, 66, 69, 76, 77, 78, 110, 113, 114, 119, and 122-125; or (iii) an LCVD of SEQ ID NO: 2 and an HCVD that is not one of SEQ ID NOs: 14, 17-22, 24, 25, 29-31, 33, 34, 40, 44, 46, 48, 49, 53, 86-96, and 98-105;
(i) an HCVD of SEQ ID NO: 147, 148, 150, 151, 154, 166, 167, 168, 169, 170, 172, 173, 174, 175, 208, 209, 210, 211, 212, 213, 214, 215, 216, 217, 218, 219, 220, 221, 222, 224, 225, 201, 203, or 228, wherein the first antigen-binding domain does not comprise (i) an HCVD of SEQ ID NO: 126 and an LCVD of SEQ ID NO: 127; (ii) an HCVD of SEQ ID NO: 126 and an LCVD that is not one of SEQ ID NOs: 181, 182, 184, 185, 187-189, 191, 199, 201, 203, and 228; (iii) an LCVD of SEQ ID NO: 127 and an HCVD that is not one of SEQ ID NOs: 147, 148, 150, 151, 154, 166-170, 172-175, 208-222, and 224-227; and
(j) an HCVD of SEQ ID NO: 238, 243, 244, 245, 246, 248, 251, 253, 255, or 267; and/or an LCVD of SEQ ID NO: 230, 275, 276, 278, 281, 283, 287, 289, 290, 292, or 297, wherein the first antigen-binding domain does not comprise (i) an HCVD of SEQ ID NO: 229 and an LCVD of SEQ ID NO: 230; (ii) an HCVD of SEQ ID NO: 229 and an LCVD that is not one of SEQ ID NOs: 275, 276, 278, 281, 283, 287, 289, 290, 292, and 297; and (iii) an LCVD of SEQ ID NO: 230 and an HCVD that is not one of SEQ ID NOs: 238, 243-246, 248, 251, 253, 255, and 267.

18. The composition of any one of claims 1-17, wherein the ABPC is degraded in the target mammalian cell following internalization of the ABPC by the target mammalian cell.

19. The composition of any one of claims 1-18, wherein the ABPC further comprises a conjugated toxin, radioisotope, drug, or small molecule.

20. The composition of claim 19, wherein the composition provides for an increase in toxin liberation in the target mammalian cell as compared to a composition comprising the same amount of a control ABPC.

21. The composition of claim 20, wherein the composition provides for at least a 20%, 50%, 2-fold, or 5-fold increase in toxin liberation in the target mammalian cell as compared to a composition comprising the same amount of a control ABPC.

22. The composition of any one of claims 19-21, wherein the composition provides for an increase in target mammalian cell killing as compared to a composition comprising the same amount of a control ABPC.

23. The composition of claim 22, wherein the composition provides for at least a 20%, 50%, 2-fold, or 5-fold increase in target mammalian cell killing as compared to a composition comprising the same amount of a control ABPC.

24. The composition of any one of claims 1-23, wherein the composition provides for an increase in endolysosomal delivery in the target mammalian cell as compared to a composition comprising the same amount of a control ABPC.

25. The composition of claim 24, wherein the composition provides for at least a 20%, 50%, 2-fold, or 5-fold increase in endolysosomal delivery in the target mammalian cell as compared to a composition comprising the same amount of a control ABPC.

26. The composition of any one of claims 1-25, wherein the composition results in a less or no detectable reduction in the level of PTK7 presented on the surface of the target mammalian cell as compared to a composition comprising the same amount of a control ABPC.

27. A composition comprising an effective amount of an antigen-binding protein construct (ABPC) comprising:
a first ABD that is capable of specifically binding PTK7 or an epitope of PTK7 presented on the surface of a target mammalian cell; and a conjugated toxin, radioisotope, drug, or small molecule,
wherein:
(a) the dissociation rate of the first ABD at a pH of ~4.0-6.5 is faster than the dissociation rate at a pH of ~7.0-8.0; or the dissociation constant (KD) of the first ABD at a pH of ~4.0-6.5 is greater than the KD at a pH of ~7.0-8.0; and
(b) the composition provides for one or more of: (i) an increase in toxin liberation in the target mammalian cell as compared to a composition comprising the same amount of a control ABPC; (ii) an increase in target mammalian cell killing as compared to a composition comprising the same amount of a control ABPC; or (iii) an increase in endolysosomal delivery in the target mammalian cell as compared to a composition comprising the same amount of a control ABPC.

28. The composition of claim 27, wherein the ABD comprises a heavy chain variable domain (HCVD) of MYT9345, MYT9359, MYT9361, MYT9412, MYT9460, MYT9792, or MYT9797, optionally with one or more histidine substitution; or an HCVD of cofetuzumab, 7C8, or 12C6, each with at least one histidine substitution.

29. The composition of claim 27, wherein the first ABD comprises a light chain variable domain (LCVD) of MYT9345, MYT9359, MYT9361, MYT9412, MYT9460, MYT9792, or MYT9797, optionally with one or more histidine substitution; or an LCVD of cofetuzumab, 7C8, or 12C6, each with at least one histidine substitution.

30. The composition of claim 27, wherein the ABD comprises one of: (a) an HCVD of MYT9345 and/or an LCVD of MYT9345; (b) an HCVD of MYT9359 and/or an LCVD of MYT9359; (c) an HCVD of MYT9361; and/or an LCVD of MYT9361; (d) an HCVD of MYT9412 and/or an LCVD of MYT9412; (e) an HCVD of MYT9460 and/or an LCVD of MYT9460; (f) an HCVD of MYT9792; and/or an LCVD of MYT9792; and (g) an HCVD of MYT9797 and/or an LCVD of MYT9797; wherein each HCVD and/or LCVD optionally has one or more histidine substitution; or, the ABD comprises one of: (h) an HCVD of cofetuzumab and/or an LCVD of cofetuzumab; (i) an HCVD of 7C8 and/or an LCVD of 7C8; and (j) an HCVD of 12C6; and/or an LCVD of 12C6, each HCVD and/or LCVD of (i), (h), or (j) having at least one histidine substitution.

31. The composition of claim 28 or 30, wherein the HCVD comprises one of: (a) an HCVD of MYT9345 comprising SEQ ID NO: 301; (b) an HCVD of MYT9359 comprising SEQ ID NO: 375; (c) an HCVD of MYT9361 comprising SEQ ID NO: 459; (d) an HCVD of MYT9412 comprising SEQ ID NO: 542; (e) an HCVD of MYT9460 comprising SEQ ID NO: 628; (f) an HCVD of MYT9792 comprising SEQ ID NO: 710; (g) an HCVD of MYT9797 comprising SEQ ID NO: 803; (h) an HCVD of cofetuzumab comprising SEQ ID NO: 1; (i) an HCVD of 7C8 comprising SEQ ID NO: 126; and (j) an HCVD of 12C6 comprising SEQ ID NO: 229.

32. The composition of claim 29 or 30, wherein the LCVD comprises one of: (a) an LCVD of MYT9345 comprising SEQ ID NO: 302; (b) an LCVD of MYT9359 comprising SEQ ID NO: 376; (c) an LCVD of MYT9361 comprising SEQ ID NO: 460; (d) an LCVD of MYT9412 comprising SEQ ID NO: 543; (e) an LCVD of MYT9460 comprising SEQ ID NO: 629; (f) an LCVD of MYT9792 comprising SEQ ID NO: 711; (g) an LCVD of MYT9797 comprising SEQ ID NO: 804; (h) an LCVD of cofetuzumab comprising SEQ ID NO: 2; (i) an LCVD of 7C8 comprising SEQ ID NO: 127; and (j) an LCVD of 12C6 comprising SEQ ID NO: 230.

33. The composition of claim 27, wherein the first ABD comprises an HCVD comprising the CDR1, 2, and 3 of one of: (a) SEQ ID NOs: 303, 304, and 305; (b) SEQ ID NOs: 377, 378, and 379; (c) SEQ ID NOs: 461, 462, and 463; (d) SEQ ID NOs: 544, 545, and 546, (e) SEQ ID NOs: 630, 631, and 632; (f) SEQ ID NOs: 712, 713, and 714; (g) SEQ ID NOs: 805, 806, and 807, (h) SEQ ID NOs: 3, 4, and 5; (i) SEQ ID NOs: 128, 129, and 130; and (j) SEQ ID NOs: 231, 232, and 233, wherein for (a) to (g), each set of three CDRs optionally has collectively one or more position substituted with a histidine, and for (h) to (j), each set of three CDRs has collectively at least one or more position substituted with a histidine.

34. The composition of claim 27, wherein the first ABD comprises an LCVD comprising the CDR1, 2, and 3 of one of: (a) SEQ ID NOs: 306, 307, and 308; (b) SEQ ID NOs: 380, 381, and 382; (c) SEQ ID NOs: 464, 465, and 466; (d) SEQ ID NOs: 547, 548, and 549, (e) SEQ ID NOs: 633, 634, and 635; (f) SEQ ID NOs: 715, 716, and 717; (g) SEQ ID NOs: 808, 809, and 810, (h) SEQ ID NOs: 6, 7, and 8; (i) SEQ ID NOs: 131, 132, and 133; and (j) SEQ ID NOs: 234, 235, and 236;
wherein for (a) to (g), each set of three CDRs optionally has collectively one or more position substituted with a histidine, and for (h) to (j), each set of three CDRs has collectively at least one or more position substituted with a histidine.

35. The composition of claim 27, wherein the first ABD comprises one of:
(a) an HCVD comprising the CDR1, 2, and 3 of SEQ ID NOs: 303, 304, and 305; and/or an LCVD comprising the CDR1, 2, and 3 of SEQ ID NOs: 306, 307, and 308;
(b) an HCVD comprising the CDR1, 2, and 3 of SEQ ID NOs: 377, 378, and 379; and/or an LCVD comprising the CDR1, 2, and 3 of SEQ ID NOs: 380, 381, and 382;
(c) an HCVD comprising the CDR1, 2, and 3 of SEQ ID NOs: 461, 462, and 463; and/or an LCVD comprising the CDR1, 2, and 3 of SEQ ID NOs: 464, 465, and 466;
(d) an HCVD comprising the CDR1, 2, and 3 of SEQ ID NOs: 544, 545, and 546; and/or an LCVD comprising the CDR1, 2, and 3 of SEQ ID NOs: 547, 548, and 549;
(e) an HCVD comprising the CDR1, 2, and 3 of SEQ ID NOs: 630, 631, and 632; and/or an LCVD comprising the CDR1, 2, and 3 of SEQ ID NOs: 633, 634, and 635;
(f) an HCVD comprising the CDR1, 2, and 3 of SEQ ID NOs: 712, 713, and 714; and/or an LCVD comprising the CDR1, 2, and 3 of SEQ ID NOs: 715, 716, and 717;
(g) an HCVD comprising the CDR1, 2, and 3 of SEQ ID NOs: 805, 806, and 807; and/or an LCVD comprising the CDR1, 2, and 3 of SEQ ID NOs: 808, 809, and 810;
(h) an HCVD comprising the CDR1, 2, and 3 of one of SEQ ID NOs: 3, 4, and 5; and/or an LCVD comprising the CDR1, 2, and 3 of SEQ ID NOs: 6, 7, and 8;
(i) an HCVD comprising the CDR1, 2, and 3 of SEQ ID NOs: 128, 129, and 130; and/or an LCVD comprising the CDR1, 2, and 3 of SEQ ID NOs: 131, 132, and 133; and
(j) an HCVD comprising the CDR1, 2, and 3 of SEQ ID NOs: 231, 232, and 233; and/or an LCVD comprising the CDR1, 2, and 3 of SEQ ID NOs: 234, 235, and 236;
wherein for (a) to (g), either or both of each set of three CDRs optionally has collectively one or more position substituted with a histidine, and
wherein for (h) to (j), either or both of each set of three CDRs has collectively at least one or more position substituted with a histidine.

36. The composition of claim 27, 28, or 33, wherein the first ABD comprises one of:
(a) an LCVD that is at least 90% identical to SEQ ID NO: 302, wherein the LCVD optionally includes a histidine at one or more positions in SEQ ID NO: 302 selected from 25, 30-33, 35, 55-57, 91, 94-96, and 98;
(b) an LCVD that is at least 90% identical to SEQ ID NO: 376, wherein the LCVD optionally includes a histidine at one or more positions in SEQ ID NO: 376 selected from 25, 29, 30, 31, 32, 33, 50, 51, 52, 53, 89, 91, 92, 93, 96, 97, 100 and 102;
(c) an LCVD that is at least 90% identical to SEQ ID NO: 460, wherein the LCVD optionally includes a histidine at one or more positions in SEQ ID NO: 460 selected from 30, 31, 33, 51, 92 and 95;
(d) an LCVD that is at least 90% identical to SEQ ID NO: 543, wherein the LCVD optionally includes a histidine at one or more positions in SEQ ID NO: 543 selected from 25, 29, 30, 31, 32, 89, 92 and 98;
(e) an LCVD that is at least 90% identical to SEQ ID NO: 629, wherein the LCVD optionally includes a histidine at one or more positions in SEQ ID NO: 629 selected from 25, 26, 28, 29, 33, 51, 55, 89, 91, 95, 96, 97, 98, 99 and 100;
(f) an LCVD that is at least 90% identical to SEQ ID NO: 711, wherein the LCVD optionally includes a histidine at one or more positions in SEQ ID NO: 711 selected from 25, 27, 29, 30, 35, 36, 56, 57, 91, 93, 94, 95, 98 and 99;
(g) an LCVD that is at least 90% identical to SEQ ID NO: 804, wherein the LCVD optionally includes a histidine at one or more positions in SEQ ID NO: 804 selected from 31, 33-35, 52, 54, 98, and 99;
(h) an LCVD that is at least 90% identical to SEQ ID NO: 2, wherein the LCVD includes a histidine at one or more positions in SEQ ID NO: 2 selected from the group consisting of: 25, 29, 31, 35, 60, 93, and 94;
(i) an LCVD that is at least 90% identical to SEQ ID NO: 127, wherein the LCVD includes a histidine at one or more positions in SEQ ID NO: 127 selected from the group consisting of: 25, 26, 28, 29, 31, 32, 33, 50, 90, 92, and 94; and
(j) an LCVD that is at least 90% identical to SEQ ID NO: 230, wherein the LCVD includes a histidine at one or more positions in SEQ ID NO: 230 selected from the group consisting of: 30, 31, 33, 51, 53, 57, 91, 92, 94, and 99.

37. The composition of claim 27, 29, or 34, wherein the first ABD comprises one of:
(a) an HCVD that is at least 90% identical to SEQ ID NO: 301, wherein the HCVD optionally includes a histidine at one or more positions in SEQ ID NO: 301 selected from 29, 30, 32, 50-54, 58, 60, 96, and 101;
(b) an HCVD that is at least 90% identical to SEQ ID NO: 375, wherein the HCVD optionally includes a histidine at one or more positions in SEQ ID NO: 375 selected from 26, 27, 30, 32, 34, 51, 52, 55, 58, 59, 63, 97, 99, and 108;
(c) an HCVD that is at least 90% identical to SEQ ID NO: 459, wherein the HCVD optionally includes a histidine at one or more positions in SEQ ID NO: 459 selected from 32, 99, 101, 102, 106 and 111;
(d) an HCVD that is at least 90% identical to SEQ ID NO: 542, wherein the HCVD optionally includes a histidine at one or more positions in SEQ ID NO: 542 selected from 27, 29, 30, 31, 32, 53, 55, 58, 102, 104 and 105;
(e) an HCVD that is at least 90% identical to SEQ ID NO: 628, wherein the HCVD optionally includes a histidine at one or more positions in SEQ ID NO: 628 selected from 26, 28, 30, 50, 52, 53, 54, 55, 56, 57, 100, 101, 102 and 103;
(f) an HCVD that is at least 90% identical to SEQ ID NO: 710, wherein the HCVD optionally includes a histidine at one or more positions in SEQ ID NO: 710 selected from 29, 30, 31, 33, 35, 50, 53, 57, 58, 65, 96, 97, 98, 100, 101, 103, 105, 107, 108, 109, 110 and 113;
(g) an HCVD that is at least 90% identical to SEQ ID NO: 803, wherein the HCVD optionally includes a histidine at one or more positions in SEQ ID NO: 803 selected from 33, 50,
(h) an HCVD that is at least 90% identical to SEQ ID NO: 1, wherein the HCVD includes a histidine at two or more positions in SEQ ID NO: 1 selected from the group consisting of: 24, 27, 28, 29, 30, 31, 32, 34, 53, 54, 55, 57, 58, 64, 98, 100, 102, 103, and 107;
(i) an HCVD that is at least 90% identical to SEQ ID NO: 126, wherein the HCVD includes a histidine at two or more positions in SEQ ID NO: 126 selected from the group consisting of: 53, 54, 56, 57, 60, 102, 103, 104, 105, 106, 108, 109, 110, and 111; and
(j) an HCVD that is at least 90% identical to SEQ ID NO: 229, wherein the HCVD includes a histidine at two or more positions in SEQ ID NO: 229 selected from the group consisting of: 27, 32, 33, 34, 35, 51, 54, 56, 58, and 100.

38. The composition of claim 27, 28, or 33, wherein the first ABD comprises one of:
(a) an LCVD that is at least 90% identical to SEQ ID NO: 302, wherein the LCVD optionally includes a histidine at two or more positions in SEQ ID NO: 302 selected from 25, 30-33, 35, 55-57, 91, 94-96, and 98;
(b) an LCVD that is at least 90% identical to SEQ ID NO: 376, wherein the LCVD optionally includes a histidine at two or more positions in SEQ ID NO: 376 selected from 25, 29, 30, 31, 32, 33, 50, 51, 52, 53, 89, 91, 92, 93, 96, 97, 100 and 102;
(c) an LCVD that is at least 90% identical to SEQ ID NO: 460, wherein the LCVD optionally includes a histidine at two or more positions in SEQ ID NO: 460 selected from 30, 31, 33, 51, 92 and 95;
(d) an LCVD that is at least 90% identical to SEQ ID NO: 543, wherein the LCVD optionally includes a histidine at two or more positions in SEQ ID NO: 543 selected from 25, 29, 30, 31, 32, 89, 92 and 98;
(e) an LCVD that is at least 90% identical to SEQ ID NO: 629, wherein the LCVD optionally includes a histidine at two or more positions in SEQ ID NO: 629 selected from 25, 26, 28, 29, 33, 51, 55, 89, 91, 95, 96, 97, 98, 99 and 100;
(f) an LCVD that is at least 90% identical to SEQ ID NO: 711, wherein the LCVD optionally includes a histidine at two or more positions in SEQ ID NO: 711 selected from 25, 27, 29, 30, 35, 36, 56, 57, 91, 93, 94, 95, 98 and 99;
(g) an LCVD that is at least 90% identical to SEQ ID NO: 804, wherein the LCVD optionally includes a histidine at two or more positions in SEQ ID NO: 804 selected from 31, 33-35, 52, 54, 98, and 99;
(h) an LCVD that is at least 90% identical to SEQ ID NO: 2, wherein the LCVD includes a histidine at two or more positions in SEQ ID NO: 2 selected from the group consisting of: 25, 29, 31, 35, 60, 93, and 94;
(i) an LCVD that is at least 90% identical to SEQ ID NO: 127, wherein the LCVD includes a histidine at two or more positions in SEQ ID NO: 127 selected from the group consisting of: 25, 26, 28, 29, 31, 32, 33, 50, 90, 92, and 94; and
(j) an LCVD that is at least 90% identical to SEQ ID NO: 230, wherein the LCVD includes a histidine at two or more positions in SEQ ID NO: 230 selected from the group consisting of: 30, 31, 33, 51, 53, 57, 91, 92, 94, and 99.

39. The composition of claim 27, 29, 34, wherein the first ABD comprises one of:
(a) an HCVD that is at least 90% identical to SEQ ID NO: 301, wherein the HCVD optionally includes a histidine at one or more positions in SEQ ID NO: 301 selected from 29, 30, 32, 50-54, 58, 60, 96, and 101; and/or an LCVD that is at least 90% identical to SEQ ID NO: 302, wherein the LCVD optionally includes a histidine at one or more positions in SEQ ID NO: 302 selected from 25, 30-33, 35, 55-57, 91, 94-96, and 98;
(b) an HCVD that is at least 90% identical to SEQ ID NO: 375, wherein the HCVD optionally includes a histidine at one or more positions in SEQ ID NO: 375 selected from 26, 27, 30, 32, 34, 51, 52, 55, 58, 59, 63, 97, 99, and 108; and/or an LCVD that is at least 90% identical to SEQ ID NO: 376, wherein the LCVD optionally includes a histidine at one or more positions in SEQ ID NO: 376 selected from 25, 29, 30, 31, 32, 33, 50, 51, 52, 53, 89, 91, 92, 93, 96, 97, 100 and 102;
(c) an HCVD that is at least 90% identical to SEQ ID NO: 459, wherein the HCVD optionally includes a histidine at one or more positions in SEQ ID NO: 459 selected from 32, 99, 101, 102, 106 and 111; and/or an LCVD that is at least 90% identical to SEQ ID NO: 460, wherein the LCVD optionally includes a histidine at one or more positions in SEQ ID NO: 460 selected from 30, 31, 33, 51, 92 and 95;
(d) an HCVD that is at least 90% identical to SEQ ID NO: 542, wherein the HCVD optionally includes a histidine at one or more positions in SEQ ID NO: 542 selected from 27, 29, 30, 31, 32, 53, 55, 58, 102, 104 and 105; and/or an LCVD that is at least 90% identical to SEQ ID NO: 543, wherein the LCVD optionally includes a histidine at one or more positions in SEQ ID NO: 543 selected from 25, 29, 30, 31, 32, 89, 92 and 98;
(e) an HCVD that is at least 90% identical to SEQ ID NO: 628, wherein the HCVD optionally includes a histidine at one or more positions in SEQ ID NO: 628 selected from 26, 28, 30, 50, 52, 53, 54, 55, 56, 57, 100, 101, 102 and 103; and/or an LCVD that is at least 90% identical to SEQ ID NO: 629, wherein the LCVD optionally includes a histidine at one or more positions in SEQ ID NO: 629 selected from 25, 26, 28, 29, 33, 51, 55, 89, 91, 95, 96, 97, 98, 99 and 100;
(f) an HCVD that is at least 90% identical to SEQ ID NO: 710, wherein the HCVD optionally includes a histidine at one or more positions in SEQ ID NO: 710 selected from 29, 30, 31, 33, 35, 50, 53, 57, 58, 65, 96, 97, 98, 100, 101, 103, 105, 107, 108, 109, 110 and 113; and/or an LCVD that is at least 90% identical to SEQ ID NO: 711, wherein the LCVD optionally includes a histidine at one or more positions in SEQ ID NO: 711 selected from 25, 27, 29, 30, 35, 36, 56, 57, 91, 93, 94, 95, 98 and 99; and
(g) an HCVD that is at least 90% identical to SEQ ID NO: 803, wherein the HCVD optionally includes a histidine at one or more positions in SEQ ID NO: 803 selected from 33, 50, 53, 55-58, 64, 100, and 107; and/or an LCVD that is at least 90% identical to SEQ ID NO: 804, wherein the LCVD optionally includes a histidine at one or more positions in SEQ ID NO: 804 selected from 31, 33-35, 52, 54, 98, and 99.
(h) an HCVD that is at least 90% identical to SEQ ID NO: 1, wherein the HCVD includes a histidine at one or more positions in SEQ ID NO: 1 selected from the group consisting of: 24, 27, 28, 29, 30, 31, 32, 34, 53, 54, 55, 57, 58, 64, 98, 100, 102, 103, and 107;
(i) an HCVD that is at least 90% identical to SEQ ID NO: 126, wherein the HCVD includes a histidine at one or more positions in SEQ ID NO: 126 selected from the group consisting of: 53, 54, 56, 57, 60, 102, 103, 104, 105, 106, 108, 109, 110, and 111; and
(j) an HCVD that is at least 90% identical to SEQ ID NO: 229, wherein the HCVD includes a histidine at one or more positions in SEQ ID NO: 229 selected from the group consisting of: 27, 32, 33, 34, 35, 51, 54, 56, 58, and 100.

40. The composition of claim 27, 30, or 35, wherein the first ABD comprises an HCVD comprising the sequence of one of SEQ ID NO: 301, or one of 309-342; SEQ ID NO: 375, or one of 383-423; SEQ ID NO: 459, or one of 467-511; SEQ ID NO: 542, or one of 550-592; SEQ ID NO: 628, or one of 636-675; SEQ ID NO: 710, or one of 718-767; and SEQ ID NO: 803, or one of 811-854; or
wherein the HCVD comprises one of the following:
(a) an HCVD of SEQ ID NO: 1, 14, 17, 18, 19, 20, 21, 22, 24, 25, 29, 30, 31, 33, 34, 40, 44, 46, 48, 49, 53, 86, 87, 88, 89, 90, 91, 92, 93, 94, 95, 96, 98, 99, 100, 101, 102, 103, 104, or, 105;
(b) an HCVD of SEQ ID NO: 126, 147, 148, 150, 151, 154, 166, 167, 168, 169, 170, 172, 173, 174, 175, 208, 209, 210, 211, 212, 213, 214, 215, 216, 217, 218, 219, 220, 221, 222, 224, 225, 226, or 227; and
(c) an HCVD of SEQ ID NO: 229, 238, 243, 244, 245, 246, 248, 251, 253, 255, or 267.

41. The composition of claim 27, wherein the first ABD comprises an LCVD comprising the sequence of one of SEQ ID NO: 302, or one of 343-374; SEQ ID NO: 376, or one of 424-458; SEQ ID NO: 460, or one of 512-541; SEQ ID NO: 543, or one of 593-627; SEQ ID NO: 629, or one of 676-709; SEQ ID NO: 711, or one of 768-854; and SEQ ID NO: 804, or one of 855-892; or
wherein the LCVD comprises one of the following:
(a) an LCVD of SEQ ID NO: 2, 56, 60, 62, 66, 69, 76, 77, 78, 110, 113, 114, 119, 122, 123, 124, or 125;
(b) an LCVD of SEQ ID NO: 127, 181, 182, 184, 185, 187, 188, 189, 191, 199, 201, 203, or 228; and
(c) an LCVD of SEQ ID NO: 230, 275, 276, 278, 281, 283, 287, 289, 290, 292, or 297.

42. The composition of claim 27 or 41, wherein the first ABD comprises:
(a) an HCVD of one of SEQ ID NO: 301, or one of 309-342, and/or an LCVD of one of SEQ ID NO: 302, or one of 343-374, wherein the first ABD does not comprise (i) SEQ ID NOs: 301 and 5; (ii) an HCVD of SEQ ID NO: 301 and an LCVD that is not one of SEQ ID NOs: 344, 349-352, 354, 359-361, 363, 366-368, 370, and 373; or (iii) an LCVD of SEQ ID NO: 302 and an HCVD that is not one of SEQ ID NO: 312, 313, 315, 319-323, 327, 329, 335, 340 and 341;
(b) an HCVD of one of SEQ ID NO: 375, or one of 383-423, and/or an LCVD of one of SEQ ID NO: 376, or one of 424-458, wherein the first ABD does not comprise (i) SEQ ID NOs: 375 and 79; (ii) an HCVD of SEQ ID NO: 375 and an LCVD that is not one of SEQ ID NOs: 425, 429, 430, 431, 432, 433, 435, 436, 437, 438, 442, 444, 445, 446, 449, 450, 453, and 455; or (iii) an LCVD of SEQ ID NO: 376 and an HCVD that is not one of SEQ ID NO: 383, 384, 387, 389, 391, 394, 395, 398, 401, 402, 406, 410, 412, and 421;
(c) an HCVD of one of SEQ ID NO: 459, or one of 467-511, and/or an LCVD of one of SEQ ID NO: 460, or one of 512-541, wherein the first ABD does not comprise (i) SEQ ID NOs: 459 and 163; (ii) an HCVD of SEQ ID NO: 459 and an LCVD that is not one of SEQ ID NOs: 518, 519, 521, 524, 533, and 536; or (iii) an LCVD of SEQ ID NO: 460 and an HCVD that is not one of SEQ ID NO: 473, 496, 498, 499, 503, and 508; and
(d) an HCVD of one of SEQ ID NO: 542, or one of 550-592, and/or an LCVD of one of SEQ ID NO: 543, or one of 593-627, wherein the first ABD does not comprise (i) SEQ ID NOs: 542 and 246; (ii) an HCVD of SEQ ID NO: 542 and an LCVD that is not one of SEQ ID NOs: 594, 598, 599, 600, 601, 611, 614, and 620; or (iii) an LCVD of SEQ ID NO: 543 and an HCVD that is not one of SEQ ID NO: 551, 553, 554, 555, 556, 563, 565, 568, 582, 584, and 585;
(e) an HCVD of one of SEQ ID NO: 628, or one of 636-675, and/or an LCVD of one of SEQ ID NO: 629, or one of 676-709, wherein the first ABD does not comprise (i) SEQ ID NOs: 628 and 332; (ii) an HCVD of SEQ ID NO: 628 and an LCVD that is not one of SEQ ID NOs: 677, 678, 680, 681, 685, 688, 692, 694, 696, 700, 701, 702, 703, 704, and 705; or (iii) an LCVD of SEQ ID NO: 629 and an HCVD that is not one of SEQ ID NO: 636, 638, 640, 646, 648, 649, 650, 651, 652, 653, 666, 667, 668, and 669;
(f) an HCVD of one of SEQ ID NO: 710, or one of 718-767, and/or an LCVD of one of SEQ ID NO: 711, or one of 768-802, wherein the first ABD does not comprise (i) SEQ ID NOs: 710 and 414; (ii) an HCVD of SEQ ID NO: 710 and an LCVD that is not one of SEQ ID NOs: 769, 771, 773, 774, 779, 780, 785, 786, 788, 790, 791, 792, 795, and 796; or (iii) an LCVD of SEQ ID NO: 711 and an HCVD that is not one of SEQ ID NO: 721, 722, 723, 725, 727, 728, 731, 735, 736, 743, 744, 745, 746, 748, 749, 751, 753, 755, 756, 757, 758, and 761;
(g) an HCVD of one of SEQ ID NO: 803, or one of 811-854, and/or an LCVD of one of SEQ ID NO: 804, or one of 855-892, wherein the first ABD does not comprise (i) SEQ ID NOs: 803 and 507; (ii) an HCVD of SEQ ID NO: 803 and an LCVD that is not one of SEQ ID NOs: 862, 864-866, 868, 870, 881, and 882; or (iii) an LCVD of SEQ ID NO: 804 and an HCVD that is not one of SEQ ID NO: 818, 821, 824, 826-829, 835, 841, 848, and 850;
(h) an HCVD of SEQ ID NO: 14, 17, 18, 19, 20, 21, 22, 24, 25, 29, 30, 31, 33, 34, 40, 44, 46, 48, 49, 53, 86, 87, 88, 89, 90, 91, 92, 93, 94, 95, 96, 98, 99, 100, 101, 102, 103, 104, or 105, and/or an LCVD of SEQ ID NO: 2, 56, 60, 62, 66, 69, 76, 77, 78, 110, 113, 114, 119, 122, 123, 124, or 125, wherein the first antigen binding domain does not comprise (i) an HCVD of SEQ ID NO: 1 and an LCVD of SEQ ID NO: 2; (ii) an HCVD of SEQ ID NO: 1 and LCVD that is not one of SEQ ID NOs: SEQ ID NOs: 56, 60, 62, 66, 69, 76, 77, 78, 110, 113, 114, 119, and 122-125; or (iii) an LCVD of SEQ ID NO: 2 and an HCVD that is not one of SEQ ID NOs: 14, 17-22, 24, 25, 29-31, 33, 34, 40, 44, 46, 48, 49, 53, 86-96, and 98-105;
(i) an HCVD of SEQ ID NO: 147, 148, 150, 151, 154, 166, 167, 168, 169, 170, 172, 173, 174, 175, 208, 209, 210, 211, 212, 213, 214, 215, 216, 217, 218, 219, 220, 221, 222, 224, 225, 226, or 227; and/or an LCVD of SEQ ID NO: 127, 181, 182, 184, 185, 187, 188, 189, 191, 199, 201, 203, or 228, wherein the first antigen-binding domain does not comprise (i) an HCVD of SEQ ID NO: 126 and an LCVD of SEQ ID NO: 127; (ii) an HCVD of SEQ ID NO: 126 and an LCVD that is not one of SEQ ID NOs: 181, 182, 184, 185, 187-189, 191, 199, 201, 203, and 228; (iii) an LCVD of SEQ ID NO: 127 and an HCVD that is not one of SEQ ID NOs: 147, 148, 150, 151, 154, 166-170, 172-175, 208-222, and 224-227; and
(j) an HCVD of SEQ ID NO: 238, 243, 244, 245, 246, 248, 251, 253, 255, or 267; and/or an LCVD of SEQ ID NO: 230, 275, 276, 278, 281, 283, 287, 289, 290, 292, or 297, wherein the first antigen-binding domain does not comprise (i) an HCVD of SEQ ID NO: 229 and an LCVD of SEQ ID NO: 230; (ii) an HCVD of SEQ ID NO: 229 and an LCVD that is not one of SEQ ID NOs: 275, 276, 278, 281, 283, 287, 289, 290, 292, and 297; and (iii) an LCVD of SEQ ID NO: 230 and an HCVD that is not one of SEQ ID NOs: 238, 243-246, 248, 251, 253, 255, and 267.

43. The composition of any one of claims 27-42, wherein the composition provides for at least a 20%, 50%, 2-fold, or 5-fold increase in toxin liberation in the target mammalian cell, and/or cell killing, and/or at least a 20%, 50%, 2-fold, or 5-fold increase in endolysosomal delivery as compared to a composition comprising the same amount of a control ABPC.

44. The composition of any one of claims 1-43, wherein the dissociation rate of the ABD at a pH of ~4.0-6.5 is at least 10%, 3-fold, or 10-fold faster than the dissociation rate of the ABD at a pH of ~7.0-8.0 and/or the K_{D} of the ABD at a pH of ~4.0-6.5 is at least 10%, 3-fold, or 10-fold greater than the KD of the ABD at a pH of ~7.0-8.0.

45. The composition of any one of claims 1-44, wherein the ABPC comprises a single polypeptide, optionally wherein the ABD is selected from a VH domain, a VHH domain, a VNAR domain, and a scFv; and/or wherein the ABPC is a BiTe, a (scFv)₂, a nanobody, a nanobody-HSA, a DART, a TandAb, a scDiabody, a scDiabody-CH3, scFv-CH-CL-scFv, a HSAbody, scDiabody-HSA, or a tandem-scFv.

46. The composition of any one of claims 1-45, wherein the ABPC comprises two or more polypeptides.

47. The composition of any one of claims 17-46, wherein at least one polypeptide of the ABPC is conjugated to the toxin, the radioisotope, the drug, or the small molecule via a cleavable or non-cleavable linker.

48. The composition of any of claims 1-47, wherein the half-life of the ABPC in vivo is increased as compared to the half-life of a control ABPC in vivo, optionally wherein the increase is ~5%-95%, ~10%-95%, ~30%-95%, ~50%-95%, or ~70%-95% as compared to the half-life of a control ABPC in vivo.

49. The composition of any one of claims 17-48, wherein the control ABPC is capable of specifically binding to PTK7 or an epitope of PTK7 presented on the surface of a target mammalian cell, wherein:
(a) the control ABPC comprises a first ABD;
(b) the dissociation rate of the first ABD of the control ABPC at a pH of ~4.0-6.5 is no more than 2-fold or 3-fold faster than the dissociation rate at a pH of ~7.0-8.0; and
(c) the dissociation constant (KD) of the first ABD of the control ABPC at a pH of ~4.0-6.5 is no more than 2-fold or 3-fold greater than the K_{D} at a pH of ~7.0-8.0.

50. The composition of any one of claims 20-49, wherein the control ABPC is MYT9345, MYT9359, MYT9361, MYT9412; MYT9460, MYT9792, MYT9797, cofetuzumab, 7C8, or 12C6.

51. The composition of any one of claims 1-50, wherein the ABPC further comprises a second ABD.

52. A kit comprising at least one dose of the composition of any one of claims 1-51.

53. An antigen-binding protein construct (ABPC) comprising: a first antigen-binding domain (ABD) that is capable of specifically binding PTK7 or an epitope of PTK7 presented on the surface of a target mammalian cell, wherein: the dissociation rate of the first ABD at a pH of ~4.0-6.5 is faster than the dissociation rate at a pH of ~7.0-8.0; or the KD of the first ABD at a pH of ~4.0-6.5 is greater than the KD at a pH of ~7.0-8.0.

54. The ABPC of claim 53, wherein the first ABD comprises an HCVD of one of an HCVD of MYT9345, MYT9359, MYT9361, MYT9412; MYT9460, MYT9792, MYT9797, cofetuzumab, 7C8, and 12C6 and/or an LCVD of one of an LCVD of MYT9345, MYT9359, MYT9361, MYT9412; MYT9460, MYT9792, MYT9797, cofetuzumab, 7C8, and 12C6, each HCVD or LCVD with one or more amino acids substituted with a histidine.

55. The ABPC of claim 54, wherein the HCVD comprises one of: (a1) an HCVD of MYT9345 comprising SEQ ID NO: 301; (b1) an HCVD of MYT9359 comprising SEQ ID NO: 375; (c1) an HCVD of MYT9361 comprising SEQ ID NO: 459; (d1) an HCVD of MYT9412 comprising SEQ ID NO: 542; (e1) an HCVD of MYT9460 comprising SEQ ID NO: 628; (f1) an HCVD of MYT9792 comprising SEQ ID NO: 710; (g1) an HCVD of MYT9797 comprising SEQ ID NO: 803; (h1) an HCVD of cofetuzumab comprising SEQ ID NO: 1; (i1) an HCVD of 7C8 comprising SEQ ID NO: 126; and (j1) an HCVD of 12C6 comprising SEQ ID NO: 229; and one of a corresponding: (a2) an LCVD of MYT9345 comprising SEQ ID NO: 302; (b2) an LCVD of MYT9359 comprising SEQ ID NO: 376; (c2) an LCVD of MYT9361 comprising SEQ ID NO: 460; (d2) an LCVD of MYT9412 comprising SEQ ID NO: 543, (e2) an LCVD of MYT9460 comprising SEQ ID NO: 629; (f2) an LCVD of MYT9792 comprising SEQ ID NO: 711; (g2) an LCVD of MYT9797 comprising SEQ ID NO: 804; (h2) an LCVD of cofetuzumab comprising SEQ ID NO: 2; (12) an LCVD of 7C8 comprising SEQ ID NO: 127; and (j2) an LCVD of 12C6 comprising SEQ ID NO: 230; wherein each HCVD and/or LCVD has one or more histidine substitution.

56. The ABPC of claim 55, wherein the first ABD comprises an HCVD comprising the CDR1, 2, and 3 of one of: (a) SEQ ID NOs: 303, 304, and 305; (b) SEQ ID NOs: 377, 378, and 379; (c) SEQ ID NOs: 461, 462, and 463; (d) SEQ ID NOs: 544, 545, and 546, (e) SEQ ID NOs: 630, 631, and 632; (f) SEQ ID NOs: 712, 713, and 714; (g) SEQ ID NOs: 805, 806, and 807, (h) SEQ ID NOs: 3, 4, and 5; (i) SEQ ID NOs: 128, 129, and 130; and (j) SEQ ID NOs: 231, 232, and 233, wherein for (a) to (g), each set of three CDRs optionally has collectively one or more position substituted with a histidine, and for (h) to (j), each set of three CDRs has collectively at least one or more position substituted with a histidine; and/or
wherein the first ABD comprises an LCVD comprising the CDR1, 2, and 3 of one of: (a) SEQ ID NOs: 306, 307, and 308; (b) SEQ ID NOs: 380, 381, and 382; (c) SEQ ID NOs: 464, 465, and 466; (d) SEQ ID NOs: 547, 548, and 549, (e) SEQ ID NOs: 633, 634, and 635; (f) SEQ ID NOs: 715, 716, and 717; (g) SEQ ID NOs: 808, 809, and 810, (h) SEQ ID NOs: 6, 7, and 8; (i) SEQ ID NOs: 131, 132, and 133; and (j) SEQ ID NOs: 234, 235, and 236; wherein for (a) to (g), each set of three CDRs optionally has collectively one or more position substituted with a histidine, and for (h) to (j), each set of three CDRs has collectively at least one or more position substituted with a histidine.

57. The ABPC of any one of claims 54-56, comprising an HCVD of one of:
(a) an HCVD that is at least 90% identical to SEQ ID NO: 301, wherein the HCVD optionally includes a histidine at one or more positions in SEQ ID NO: 301 selected from: 29, 30, 32, 50-54, 58, 60, 96, and 101;
(b) an HCVD that is at least 90% identical to SEQ ID NO: 375, wherein the HCVD optionally includes a histidine at one or more positions in SEQ ID NO: 375 selected from: 26, 27, 30, 32, 34, 51, 52, 55, 58, 59, 63, 97, 99, and 108;
(c) an HCVD that is at least 90% identical to SEQ ID NO: 459, wherein the HCVD optionally includes a histidine at one or more positions in SEQ ID NO: 459 selected from: 32, 99, 101, 102, 106 and 111;
(d) an HCVD that is at least 90% identical to SEQ ID NO: 542, wherein the HCVD optionally includes a histidine at one or more positions in SEQ ID NO: 542 selected from: 27, 29, 30, 31, 32, 53, 55, 58, 102, 104 and 105;
(e) an HCVD that is at least 90% identical to SEQ ID NO: 628, wherein the HCVD optionally includes a histidine at one or more positions in SEQ ID NO: 628 selected from: 26, 28, 30, 50, 52, 53, 54, 55, 56, 57, 100, 101, 102 and 103;
(f) an HCVD that is at least 90% identical to SEQ ID NO: 710, wherein the HCVD optionally includes a histidine at one or more positions in SEQ ID NO: 710 selected from: 29, 30, 31, 33, 35, 50, 53, 57, 58, 65, 96, 97, 98, 100, 101, 103, 105, 107, 108, 109, 110 and 113; and
(g) an HCVD that is at least 90% identical to SEQ ID NO: 803, wherein the HCVD optionally includes a histidine at one or more positions in SEQ ID NO: 803 selected from: 33,
(h) an HCVD that is at least 90% identical to SEQ ID NO: 1, wherein the HCVD includes a histidine at one or more positions in SEQ ID NO: 1 selected from the group consisting of: 24, 27, 28, 29, 30, 31, 32, 34, 53, 54, 55, 57, 58, 64, 98, 100, 102, 103, and 107;
(i) an HCVD that is at least 90% identical to SEQ ID NO: 126, wherein the HCVD includes a histidine at one or more positions in SEQ ID NO: 126 selected from the group consisting of: 53, 54, 56, 57, 60, 102, 103, 104, 105, 106, 108, 109, 110, and 111; and
(j) an HCVD that is at least 90% identical to SEQ ID NO: 229, wherein the HCVD includes a histidine at one or more positions in SEQ ID NO: 229 selected from the group consisting of: 27, 32, 33, 34, 35, 51, 54, 56, 58, and 100.

58. The ABPC of any one of claims 54-56, comprising an LCVD of one of:
(a) an LCVD that is at least 90% identical to SEQ ID NO: 302, wherein the LCVD optionally includes a histidine at one or more positions in SEQ ID NO: 302 selected from: 25, 30-33, 35, 55-57, 91, 94-96, and 98;
(b) an LCVD that is at least 90% identical to SEQ ID NO: 376, wherein the LCVD optionally includes a histidine at one or more positions in SEQ ID NO: 376 selected from: 25, 29, 30, 31, 32, 33, 50, 51, 52, 53, 89, 91, 92, 93, 96, 97, 100 and 102;
(c) an LCVD that is at least 90% identical to SEQ ID NO: 460, wherein the LCVD optionally includes a histidine at one or more positions in SEQ ID NO: 460 selected from: 30, 31, 33, 51, 92 and 95;
(d) an LCVD that is at least 90% identical to SEQ ID NO: 543, wherein the LCVD optionally includes a histidine at one or more positions in SEQ ID NO: 543 selected from: 25, 29, 30, 31, 32, 89, 92 and 98;
(e) an LCVD that is at least 90% identical to SEQ ID NO: 629, wherein the LCVD optionally includes a histidine at one or more positions in SEQ ID NO: 629 selected from: 25, 26, 28, 29, 33, 51, 55, 89, 91, 95, 96, 97, 98, 99 and 100;
(f) an LCVD that is at least 90% identical to SEQ ID NO: 711, wherein the LCVD optionally includes a histidine at one or more positions in SEQ ID NO: 711 selected from: 25, 27, 29, 30, 35, 36, 56, 57, 91, 93, 94, 95, 98 and 99; and
(g) an LCVD that is at least 90% identical to SEQ ID NO: 804, wherein the LCVD optionally includes a histidine at one or more positions in SEQ ID NO: 804 selected from: 31, 33-35, 52, 54, 98, and 99;
(h) an LCVD that is at least 90% identical to SEQ ID NO: 2, wherein the LCVD includes a histidine at one or more positions in SEQ ID NO: 2 selected from the group consisting of: 25, 29, 31, 35, 60, 93, and 94;
(i) an LCVD that is at least 90% identical to SEQ ID NO: 127, wherein the LCVD includes a histidine at one or more positions in SEQ ID NO: 127 selected from the group consisting of: 25, 26, 28, 29, 31, 32, 33, 50, 90, 92, and 94; and
(j) an LCVD that is at least 90% identical to SEQ ID NO: 230, wherein the LCVD includes a histidine at one or more positions in SEQ ID NO: 230 selected from the group consisting of: 30, 31, 33, 51, 53, 57, 91, 92, 94, and 99.

59. The ABPC of 57 or 58, wherein the HCVD and/or LCVD optionally includes a histidine at two or more positions in its defining SEQ ID NO.

60. The ABPC of claim 53, wherein the first ABD comprises an HCVD comprising the sequence of one of SEQ ID NO: 301, or one of 309-342; SEQ ID NO: 375, or one of 383-423; SEQ ID NO: 459, or one of 467-511; SEQ ID NO: 542, or one of 550-592; SEQ ID NO: 628, or one of 636-675; SEQ ID NO: 710, or one of 718-767; and SEQ ID NO: 803, or one of 811-854; or wherein the HCVD comprises one of the following: (a) an HCVD of SEQ ID NO: 1, 14, 17, 18, 19, 20, 21, 22, 24, 25, 29, 30, 31, 33, 34, 40, 44, 46, 48, 49, 53, 86, 87, 88, 89, 90, 91, 92, 93, 94, 95, 96, 98, 99, 100, 101, 102, 103, 104, or, 105; (b) an HCVD of SEQ ID NO: 126, 147, 148, 150, 151, 154, 166, 167, 168, 169, 170, 172, 173, 174, 175, 208, 209, 210, 211, 212, 213, 214, 215, 216, 217, 218, 219, 220, 221, 222, 224, 225, 226, or 227; and (c) an HCVD of SEQ ID NO: 229, 238, 243, 244, 245, 246, 248, 251, 253, 255, or 267.

61. The ABPC of claim 53, wherein the first ABD comprises one of:
(a) an HCVD of one of SEQ ID NO: 301, or one of 309-342, and/or an LCVD of one of SEQ ID NO: 302, or one of 343-374, wherein the first ABD does not comprise (i) SEQ ID NOs: 301 and 5; (ii) an HCVD of SEQ ID NO: 301 and an LCVD that is not one of SEQ ID NOs: 344, 349-352, 354, 359-361, 363, 366-368, 370, and 373; or (iii) an LCVD of SEQ ID NO: 302 and an HCVD that is not one of SEQ ID NO: 312, 313, 315, 319-323, 327, 329, 335, 340 and 341;
(b) an HCVD of one of SEQ ID NO: 375, or one of 383-423, and/or an LCVD of one of SEQ ID NO: 376, or one of 424-458, wherein the first ABD does not comprise (i) SEQ ID NOs: 375 and 79; (ii) an HCVD of SEQ ID NO: 375 and an LCVD that is not one of SEQ ID NOs: 425, 429, 430, 431, 432, 433, 435, 436, 437, 438, 442, 444, 445, 446, 449, 450, 453, and 455; or (iii) an LCVD of SEQ ID NO: 376 and an HCVD that is not one of SEQ ID NO: 383, 384, 387, 389, 391, 394, 395, 398, 401, 402, 406, 410, 412, and 421;
(c) an HCVD of one of SEQ ID NO: 459, or one of 467-511, and/or an LCVD of one of SEQ ID NO: 460, or one of 512-541, wherein the first ABD does not comprise (i) SEQ ID NOs: 459 and 163; (ii) an HCVD of SEQ ID NO: 459 and an LCVD that is not one of SEQ ID NOs: 518, 519, 521, 524, 533, and 536; or (iii) an LCVD of SEQ ID NO: 460 and an HCVD that is not one of SEQ ID NO: 473, 496, 498, 499, 503, and 508;
(d) an HCVD of one of SEQ ID NO: 542, or one of 550-592, and/or an LCVD of one of SEQ ID NO: 543, or one of 593-627, wherein the first ABD does not comprise (i) SEQ ID NOs: 542 and 246; (ii) an HCVD of SEQ ID NO: 542 and an LCVD that is not one of SEQ ID NOs: 594, 598, 599, 600, 601, 611, 614, and 620; or (iii) an LCVD of SEQ ID NO: 543 and an HCVD that is not one of SEQ ID NO: 551, 553, 554, 555, 556, 563, 565, 568, 582, 584, and 585;
(e) an HCVD of one of SEQ ID NO: 628, or one of 636-675, and/or an LCVD of one of SEQ ID NO: 629, or one of 676-709, wherein the first ABD does not comprise (i) SEQ ID NOs: 628 and 332; (ii) an HCVD of SEQ ID NO: 628 and an LCVD that is not one of SEQ ID NOs: 677, 678, 680, 681, 685, 688, 692, 694, 696, 700, 701, 702, 703, 704, and 705; or (iii) an LCVD of SEQ ID NO: 629 and an HCVD that is not one of SEQ ID NO: 636, 638, 640, 646, 648, 649, 650, 651, 652, 653, 666, 667, 668, and 669;
(f) an HCVD of one of SEQ ID NO: 710, or one of 718-767, and/or an LCVD of one of SEQ ID NO: 711, or one of 768-802, wherein the first ABD does not comprise (i) SEQ ID NOs: 710 and 414; (ii) an HCVD of SEQ ID NO: 710 and an LCVD that is not one of SEQ ID NOs: 769, 771, 773, 774, 779, 780, 785, 786, 788, 790, 791, 792, 795, and 796; or (iii) an LCVD of SEQ ID NO: 711 and an HCVD that is not one of SEQ ID NO: 721, 722, 723, 725, 727, 728, 731, 735, 736, 743, 744, 745, 746, 748, 749, 751, 753, 755, 756, 757, 758, and 761;
(g) an HCVD of one of SEQ ID NO: 803, or one of 811-854, and/or an LCVD of one of SEQ ID NO: 804, or one of 855-892, wherein the first ABD does not comprise (i) SEQ ID NOs: 803 and 507; (ii) an HCVD of SEQ ID NO: 803 and an LCVD that is not one of SEQ ID NOs: 862, 864-866, 868, 870, 881, and 882; or (iii) an LCVD of SEQ ID NO: 804 and an HCVD that is not one of SEQ ID NO: 818, 821, 824, 826-829, 835, 841, 848, and 850;
(h) an HCVD of SEQ ID NO: 14, 17, 18, 19, 20, 21, 22, 24, 25, 29, 30, 31, 33, 34, 40, 44, 46, 48, 49, 53, 86, 87, 88, 89, 90, 91, 92, 93, 94, 95, 96, 98, 99, 100, 101, 102, 103, 104, or 105, and/or an LCVD of SEQ ID NO: 2, 56, 60, 62, 66, 69, 76, 77, 78, 110, 113, 114, 119, 122, 123, 124, or 125, wherein the first antigen binding domain does not comprise (i) an HCVD of SEQ ID NO: 1 and an LCVD of SEQ ID NO: 2; (ii) an HCVD of SEQ ID NO: 1 and an LCVD that is not one of SEQ ID NOs: SEQ ID NOs: 56, 60, 62, 66, 69, 76, 77, 78, 110, 113, 114, 119, and 122-125; or (iii) an LCVD of SEQ ID NO: 2 and an HCVD that is not one of SEQ ID NOs: 14, 17-22, 24, 25, 29-31, 33, 34, 40, 44, 46, 48, 49, 53, 86-96, and 98-105;
(i) an HCVD of SEQ ID NO: 147, 148, 150, 151, 154, 166, 167, 168, 169, 170, 172, 173, 174, 175, 208, 209, 210, 211, 212, 213, 214, 215, 216, 217, 218, 219, 220, 221, 222, 224, 225, 226, or 227; and/or an LCVD of SEQ ID NO: 127, 181, 182, 184, 185, 187, 188, 189, 191, 199, 201, 203, or 228, wherein the first antigen-binding domain does not comprise (i) an HCVD of SEQ ID NO: 126 and an LCVD of SEQ ID NO: 127; (ii) an HCVD of SEQ ID NO: 126 and an LCVD that is not one of SEQ ID NOs: 181, 182, 184, 185, 187-189, 191, 199, 201, 203, and 228; (iii) an LCVD of SEQ ID NO: 127 and an HCVD that is not one of SEQ ID NOs: 147, 148, 150, 151, 154, 166-170, 172-175, 208-222, and 224-227; and
(j) an HCVD of SEQ ID NO: 238, 243, 244, 245, 246, 248, 251, 253, 255, or 267; and/or an LCVD of SEQ ID NO: 230, 275, 276, 278, 281, 283, 287, 289, 290, 292, or 297, wherein the first antigen-binding domain does not comprise (i) an HCVD of SEQ ID NO: 229 and an LCVD of SEQ ID NO: 230; (ii) an HCVD of SEQ ID NO: 229 and an LCVD that is not one of SEQ ID NOs: 275, 276, 278, 281, 283, 287, 289, 290, 292, and 297; and (iii) an LCVD of SEQ ID NO: 230 and an HCVD that is not one of SEQ ID NOs: 238, 243-246, 248, 251, 253, 255, and 267.

62. The ABPC of any one of claims 53-61, wherein the ABPC is degraded in the target mammalian cell following internalization of the ABPC by the cell; and/or wherein the ABPC further comprises a conjugated toxin, radioisotope, drug, or small molecule; and/or wherein the ABPC provides for an increase in toxin liberation in the cell as compared to the same amount of a control ABPC.

63. The ABPC of claim 62, wherein the ABPC provides for at least a 20%, 50%, 2-fold, or 5-fold increase in toxin liberation; and/or wherein the ABPC provides for an increase in target mammalian cell killing as compared to the same amount of a control ABPC.

64. The ABPC of claim 63, wherein the composition provides for at least a 20%, 50%, 2-fold, or 5-fold increase in target mammalian cell killing as compared to the same amount of a control ABPC.

65. The ABPC of any one of claims 53-64, wherein the ABPC provides for an increase in endolysosomal delivery in the target mammalian cell as compared to the same amount of a control ABPC; optionally wherein the increase is at least a 20%, 50%, 2-fold, or 5-fold increase.

66. The ABPC of any one of claims 53-65, wherein the ABPC results in a less or no detectable reduction in the level of PTK7 presented on the surface of the target mammalian cell as compared to the same amount of a control ABPC.

67. The ABPC of any one of claims 53-66, wherein the ABPC further comprises a second ABD, and/or wherein the ABPC is conjugated to a drug.

68. A kit comprising at least one dose of the ABPC of any one of claims 53-67.

69. A method of treating a cancer characterized by having a population of cancer cells that have a predetermined level of PTK7 or an epitope of PTK7 presented on their surface, comprising administering a therapeutically effective amount of the composition of any one of claims 1-51 or the ABPC of any one of claims 53-68 to a subject identified as having a cancer characterized by having the population of cancer cells.

70. A method of reducing the volume of a tumor in a subject, wherein the tumor is characterized by having PTK7 positive cancer cells, comprising administering a therapeutically effective amount of the composition of any one of claims 1-51 or the ABPC of any one of claims 53-67 to the subject identified as having a cancer characterized by the cancer cells.

71. A method of inducing cell death in a PTK7 positive cancer cell in a subject, comprising administering a therapeutically effective amount of the composition of any one of claims 1-51 or the ABPC of any one of claims 53-67 to a subject identified as having the cancer.

72. A method of decreasing the risk of developing a metastasis or an additional metastasis in a subject having a PTK7 positive cancer, comprising administering a therapeutically effective amount of the composition of any one of claims 1-51 or the ABPC of any one of claims 53-67 to the subject identified as having the cancer.


SEQUENCE LISTING (892 sequences)

SEQ ID NO: 1 - protein, 119 aa, synthetic construct
  REGION 1..119: note = Heavy Chain of cofetuzumab IgG
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 2 - protein, 111 aa, synthetic construct
  REGION 1..111: note = Light Chain of cofetuzumab IgG
EIVLTQSPATLSLSPGERATLSCRASESVDSYGKSFMHWYQQKPGQAPRLLIYRASNLESGIPARFSGSGSGTDFTLTISSLEPEDFAVYYCQQSNEDPWTFGGGTKLEIK

SEQ ID NO: 3 - protein, 13 aa, synthetic construct
  REGION 1..13: note = Heavy Chain CDR1 of cofetuzumab IgG
KASGYTFTDYAVH

SEQ ID NO: 4 - protein, 17 aa, synthetic construct
  REGION 1..17: note = Heavy Chain CDR2 of cofetuzumab IgG
VISTYNDYTYNNQDFKG

SEQ ID NO: 5 - protein, 12 aa, synthetic construct
  REGION 1..12: note = Heavy Chain CDR3 of cofetuzumab IgG
ARGNSYFYALDY

SEQ ID NO: 6 - protein, 15 aa, synthetic construct
  REGION 1..15: note = Light Chain CDR1 of cofetuzumab IgG
RASESVDSYGKSFMH

SEQ ID NO: 7 - protein, 7 aa, synthetic construct
  REGION 1..7: note = Light Chain CDR2 of cofetuzumab IgG
RASNLES

SEQ ID NO: 8 - protein, 9 aa, synthetic construct
  REGION 1..9: note = Light Chain CDR3 of cofetuzumab IgG
QQSNEDPWT

SEQ ID NO: 9 - protein, 1070 aa, synthetic construct
  REGION 1..1070: note = Mature Human PTK7
MGAARGSPARPRRLPLLSVLLLPLLGGTQTAIVFIKQPSSQDALQGRRALLRCEVEAPGPVHVYWLLDGAPVQDTERRFAQGSSLSFAAVDRLQDSGTFQCVARDDVTGEEARSANASFNIKWIEAGPVVLKHPASEAEIQPQTQVTLRCHIDGHPRPTYQWFRDGTPLSDGQSNHTVSSKERNLTLRPAGPEHSGLYSCCAHSAFGQACSSQNFTLSIADESFARVVLAPQDVVVARYEEAMFHCQFSAQPPPSLQWLFEDETPITNRSRPPHLRRATVFANGSLLLTQVRPRNAGIYRCIGQGQRGPPIILEATLHLAEIEDMPLFEPRVFTAGSEERVTCLPPKGLPEPSVWWEHAGVRLPTHGRVYQKGHELVLANIAESDAGVYTCHAANLAGQRRQDVNITVATVPSWLKKPQDSQLEEGKPGYLDCLTQATPKPTVVWYRNQMLISEDSRFEVFKNGTLRINSVEVYDGTWYRCMSSTPAGSIEAQARVQVLEKLKFTPPPQPQQCMEFDKEATVPCSATGREKPTIKWERADGSSLPEWVTDNAGTLHFARVTRDDAGNYTCIASNGPQGQIRAHVQLTVAVFITFKVEPERTTVYQGHTALLQCEAQGDPKPLIQWKGKDRILDPTKLGPRMHIFQNGSLVIHDVAPEDSGRYTCIAGNSCNIKHTEAPLYVVDKPVPEESEGPGSPPPYKMIQTIGLSVGAAVAYIIAVLGLMFYCKKRCKAKRLQKQPEGEEPEMECLNGGPLQNGQPSAEIQEEVALTSLGSGPAATNKRHSTSDKMHFPRSSLQPITTLGKSEFGEVFLAKAQGLEEGVAETLVLVKSLQSKDEQQQLDFRRELEMFGKLNHANVVRLLGLCREAEPHYMVLEYVDLGDLKQFLRISKSKDEKLKSQPLSTKQKVALCTQVALGMEHLSNNRFVHKDLAARNCLVSAQRQVKVSALGLSKDVYNSEYYHFRQAWVPLRWMSPEAILEGDFSTKSDVWAFGVLMWEVFTHGEMPHGGQADDEVLADLQAGKARLPQPEGCPSKLYRLMQRCWALSPKDRPSFSEIASALGDSTVDSKP

SEQ ID NO: 10 - DNA, 3210 bp, synthetic construct
  misc_feature 1..3210: note = cDNA encoding Mature Human PTK7
  source 1..3210: mol_type = other DNA
atgggagctgcgcggggatccccggccagaccccgccggttgcctctgctcagcgtcctgctgctgccgctgctgggcggtacccagacagccattgtcttcatcaagcagccgtcctcccaggatgcactgcaggggcgccgggcgctgcttcgctgtgaggttgaggctccgggcccggtacatgtgtactggctgctcgatggggcccctgtccaggacacggagcggcgtttcgcccagggcagcagcctgagctttgcagctgtggaccggctgcaggactctggcaccttccagtgtgtggctcgggatgatgtcactggagaagaagcccgcagtgccaacgcctccttcaacatcaaatggattgaggcaggtcctgtggtcctgaagcatccagcctcggaagctgagatccagccacagacccaggtcacacttcgttgccacattgatgggcaccctcggcccacctaccaatggttccgagatgggacccccctttctgatggtcagagcaaccacacagtcagcagcaaggagcggaacctgacgctccggccagctggtcctgagcatagtgggctgtattcctgctgcgcccacagtgcttttggccaggcttgcagcagccagaacttcaccttgagcattgctgatgaaagctttgccagggtggtgctggcaccccaggacgtggtagtagcgaggtatgaggaggccatgttccattgccagttctcagcccagccacccccgagcctgcagtggctctttgaggatgagactcccatcactaaccgcagtcgccccccacacctccgcagagccacagtgtttgccaacgggtctctgctgctgacccaggtccggccacgcaatgcagggatctaccgctgcattggccaggggcagaggggcccacccatcatcctggaagccacacttcacctagcagagattgaagacatgccgctatttgagccacgggtgtttacagctggcagcgaggagcgtgtgacctgccttccccccaagggtctgccagagcccagcgtgtggtgggagcacgcgggagtccggctgcccacccatggcagggtctaccagaagggccacgagctggtgttggccaatattgctgaaagtgatgctggtgtctacacctgccacgcggccaacctggctggtcagcggagacaggatgtcaacatcactgtggccactgtgccctcctggctgaagaagccccaagacagccagctggaggagggcaaacccggctacttggattgcctgacccaggccacaccaaaacctacagttgtctggtacagaaaccagatgctcatctcagaggactcacggttcgaggtcttcaagaatgggaccttgcgcatcaacagcgtggaggtgtatgatgggacatggtaccgttgtatgagcagcaccccagccggcagcatcgaggcgcaagcccgtgtccaagtgctggaaaagctcaagttcacaccaccaccccagccacagcagtgcatggagtttgacaaggaggccacggtgccctgttcagccacaggccgagagaagcccactattaagtgggaacgggcagatgggagcagcctcccagagtgggtgacagacaacgctgggaccctgcattttgcccgggtgactcgagatgacgctggcaactacacttgcattgcctccaacgggccgcagggccagattcgtgcccatgtccagctcactgtggcagtttttatcaccttcaaagtggaaccagagcgtacgactgtgtaccagggccacacagccctactgcagtgcgaggcccagggggaccccaagccgctgattcagtggaaaggcaaggaccgcatcctggaccccaccaagctgggacccaggatgcacatcttccagaatggctccctggtgatccatgacgtggcccctgaggactcaggccgctacacctgcattgcaggcaacagctgcaacatcaagcacacggaggcccccctctatgtcgtggacaagcctgtgccggaggagtcggagggccctggcagccctcccccctacaagatgatccagaccattgggttgtcggtgggtgccgctgtggcctacatcattgccgtgctgggcctcatgttctactgcaagaagcgctgcaaagccaagcggctgcagaagcagcccgagggcgaggagccagagatggaatgcctcaacggtgggcctttgcagaacgggcagccctcagcagagatccaagaagaagtggccttgaccagcttgggctccggccccgcggccaccaacaaacgccacagcacaagtgataagatgcacttcccacggtctagcctgcagcccatcaccacgctggggaagagtgagtttggggaggtgttcctggcaaaggctcagggcttggaggagggagtggcagagaccctggtacttgtgaagagcctgcagagcaaggatgagcagcagcagctggacttccggagggagttggagatgtttgggaagctgaaccacgccaacgtggtgcggctcctggggctgtgccgggaggctgagccccactacatggtgctggaatatgtggatctgggagacctcaagcagttcctgaggatttccaagagcaaggatgaaaaattgaagtcacagcccctcagcaccaagcagaaggtggccctatgcacccaggtagccctgggcatggagcacctgtccaacaaccgctttgtgcataaggacttggctgcgcgtaactgcctggtcagtgcccagagacaagtgaaggtgtctgccctgggcctcagcaaggatgtgtacaacagtgagtactaccacttccgccaggcctgggtgccgctgcgctggatgtcccccgaggccatcctggagggtgacttctctaccaagtctgatgtctgggccttcggtgtgctgatgtgggaagtgtttacacatggagagatgccccatggtgggcaggcagatgatgaagtactggcagatttgcaggctgggaaggctagacttcctcagcccgagggctgcccttccaaactctatcggctgatgcagcgctgctgggccctcagccccaaggaccggccctccttcagtgagattgccagcgccctgggagacagcaccgtggacagcaagccg

SEQ ID NO: 11 - protein, 674 aa, synthetic construct
  REGION 1..674: note = Extracellular domain of PTK7
AIVFIKQPSSQDALQGRRALLRCEVEAPGPVHVYWLLDGAPVQDTERRFAQGSSLSFAAVDRLQDSGTFQCVARDDVTGEEARSANASFNIKWIEAGPVVLKHPASEAEIQPQTQVTLRCHIDGHPRPTYQWFRDGTPLSDGQSNHTVSSKERNLTLRPAGPEHSGLYSCCAHSAFGQACSSQNFTLSIADESFARVVLAPQDVVVARYEEAMFHCQFSAQPPPSLQWLFEDETPITNRSRPPHLRRATVFANGSLLLTQVRPRNAGIYRCIGQGQRGPPIILEATLHLAEIEDMPLFEPRVFTAGSEERVTCLPPKGLPEPSVWWEHAGVRLPTHGRVYQKGHELVLANIAESDAGVYTCHAANLAGQRRQDVNITVATVPSWLKKPQDSQLEEGKPGYLDCLTQATPKPTVVWYRNQMLISEDSRFEVFKNGTLRINSVEVYDGTWYRCMSSTPAGSIEAQARVQVLEKLKFTPPPQPQQCMEFDKEATVPCSATGREKPTIKWERADGSSLPEWVTDNAGTLHFARVTRDDAGNYTCIASNGPQGQIRAHVQLTVAVFITFKVEPERTTVYQGHTALLQCEAQGDPKPLIQWKGKDRILDPTKLGPRMHIFQNGSLVIHDVAPEDSGRYTCIAGNSCNIKHTEAPLYVVDKPVPEESEGPGSPPPYKMIQT

SEQ ID NO: 12 - DNA, 2022 bp, synthetic construct
  misc_feature 1..2022: note = cDNA encoding Extracellular domain of PTK7
  source 1..2022: mol_type = other DNA
gccattgtcttcatcaagcagccgtcctcccaggatgcactgcaggggcgccgggcgctgcttcgctgtgaggttgaggctccgggcccggtacatgtgtactggctgctcgatggggcccctgtccaggacacggagcggcgtttcgcccagggcagcagcctgagctttgcagctgtggaccggctgcaggactctggcaccttccagtgtgtggctcgggatgatgtcactggagaagaagcccgcagtgccaacgcctccttcaacatcaaatggattgaggcaggtcctgtggtcctgaagcatccagcctcggaagctgagatccagccacagacccaggtcacacttcgttgccacattgatgggcaccctcggcccacctaccaatggttccgagatgggacccccctttctgatggtcagagcaaccacacagtcagcagcaaggagcggaacctgacgctccggccagctggtcctgagcatagtgggctgtattcctgctgcgcccacagtgcttttggccaggcttgcagcagccagaacttcaccttgagcattgctgatgaaagctttgccagggtggtgctggcaccccaggacgtggtagtagcgaggtatgaggaggccatgttccattgccagttctcagcccagccacccccgagcctgcagtggctctttgaggatgagactcccatcactaaccgcagtcgccccccacacctccgcagagccacagtgtttgccaacgggtctctgctgctgacccaggtccggccacgcaatgcagggatctaccgctgcattggccaggggcagaggggcccacccatcatcctggaagccacacttcacctagcagagattgaagacatgccgctatttgagccacgggtgtttacagctggcagcgaggagcgtgtgacctgccttccccccaagggtctgccagagcccagcgtgtggtgggagcacgcgggagtccggctgcccacccatggcagggtctaccagaagggccacgagctggtgttggccaatattgctgaaagtgatgctggtgtctacacctgccacgcggccaacctggctggtcagcggagacaggatgtcaacatcactgtggccactgtgccctcctggctgaagaagccccaagacagccagctggaggagggcaaacccggctacttggattgcctgacccaggccacaccaaaacctacagttgtctggtacagaaaccagatgctcatctcagaggactcacggttcgaggtcttcaagaatgggaccttgcgcatcaacagcgtggaggtgtatgatgggacatggtaccgttgtatgagcagcaccccagccggcagcatcgaggcgcaagcccgtgtccaagtgctggaaaagctcaagttcacaccaccaccccagccacagcagtgcatggagtttgacaaggaggccacggtgccctgttcagccacaggccgagagaagcccactattaagtgggaacgggcagatgggagcagcctcccagagtgggtgacagacaacgctgggaccctgcattttgcccgggtgactcgagatgacgctggcaactacacttgcattgcctccaacgggccgcagggccagattcgtgcccatgtccagctcactgtggcagtttttatcaccttcaaagtggaaccagagcgtacgactgtgtaccagggccacacagccctactgcagtgcgaggcccagggggaccccaagccgctgattcagtggaaaggcaaggaccgcatcctggaccccaccaagctgggacccaggatgcacatcttccagaatggctccctggtgatccatgacgtggcccctgaggactcaggccgctacacctgcattgcaggcaacagctgcaacatcaagcacacggaggcccccctctatgtcgtggacaagcctgtgccggaggagtcggagggccctggcagccctcccccctacaagatgatccagacc

SEQ ID NO: 13 - protein, 119 aa, synthetic construct
  REGION 1..119: note = Heavy Chain of cofetuzumab IgG histidine scanning variant #1
QVQLVQSGPEVKKPGASVKVSCHASGYTFTDYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 14 - protein, 119 aa, synthetic construct
  REGION 1..119: note = Heavy Chain of cofetuzumab IgG histidine scanning variant #2
QVQLVQSGPEVKKPGASVKVSCKHSGYTFTDYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 15 - protein, 119 aa, synthetic construct
  REGION 1..119: note = Heavy Chain of cofetuzumab IgG histidine scanning variant #3
QVQLVQSGPEVKKPGASVKVSCKAHGYTFTDYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 16 - protein, 119 aa, synthetic construct
  REGION 1..119: note = Heavy Chain of cofetuzumab IgG histidine scanning variant #4
QVQLVQSGPEVKKPGASVKVSCKASHYTFTDYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 17 - protein, 119 aa, synthetic construct
  REGION 1..119: note = Heavy Chain of cofetuzumab IgG histidine scanning variant #5
QVQLVQSGPEVKKPGASVKVSCKASGHTFTDYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 18 - protein, 119 aa, synthetic construct
  REGION 1..119: note = Heavy Chain of cofetuzumab IgG histidine scanning variant #6
QVQLVQSGPEVKKPGASVKVSCKASGYHFTDYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 19 - protein, 119 aa, synthetic construct
  REGION 1..119: note = Heavy Chain of cofetuzumab IgG histidine scanning variant #7
QVQLVQSGPEVKKPGASVKVSCKASGYTHTDYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 20 - protein, 119 aa, synthetic construct
  REGION 1..119: note = Heavy Chain of cofetuzumab IgG histidine scanning variant #8
QVQLVQSGPEVKKPGASVKVSCKASGYTFHDYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 21 - protein, 119 aa, synthetic construct
  REGION 1..119: note = Heavy Chain of cofetuzumab IgG histidine scanning variant #9
QVQLVQSGPEVKKPGASVKVSCKASGYTFTHYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 22 - protein, 119 aa, synthetic construct
  REGION 1..119: note = Heavy Chain of cofetuzumab IgG histidine scanning variant #10
QVQLVQSGPEVKKPGASVKVSCKASGYTFTDHAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 23 - protein, 119 aa, synthetic construct
  REGION 1..119: note = Heavy Chain of cofetuzumab IgG histidine scanning variant #11
QVQLVQSGPEVKKPGASVKVSCKASGYTFTDYHVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 24 - protein, 119 aa, synthetic construct
  REGION 1..119: note = Heavy Chain of cofetuzumab IgG histidine scanning variant #12
QVQLVQSGPEVKKPGASVKVSCKASGYTFTDYAHHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 25 - protein, 119 aa, synthetic construct
  REGION 1..119: note = Heavy Chain of cofetuzumab IgG histidine scanning variant #13
QVQLVQSGPEVKKPGASVKVSCKASGYTFTDYAVAWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 26 - protein, 119 aa, synthetic construct
  REGION 1..119: note = Heavy Chain of cofetuzumab IgG histidine scanning variant #14
QVQLVQSGPEVKKPGASVKVSCKASGYTFTDYAVHWVRQAPGKRLEWIGHISTYNDYTYNNQDFKGRVTMTRDTSASTAYMELSRLRSEDTAVYYCARGNSYFYALDYWGQGTSVTVSS

SEQ ID NO: 27 - protein, 119 aa, synthetic construct
  REGION 1..119: note = Heavy Chain of cofetuzumab IgG histidine scanning variant #15
QVQLVQSGPEVKKPGASVKVSCKASGYTFTDYAVHWVRQAPGKRLEWIGVHSTYNDYTYNNQDFKGRVTMTRDTSASTAYMELSRLRSEDTAVYYCARGNSYFYALDYWGQGTSVTVSS

SEQ ID NO: 28 - protein, 119 aa, synthetic construct
  REGION 1..119: note = Heavy Chain of cofetuzumab IgG histidine scanning variant #16
QVQLVQSGPEVKKPGASVKVSCKASGYTFTDYAVHWVRQAPGKRLEWIGVIHTYNDYTYNNQDFKGRVTMTRDTSASTAYMELSRLRSEDTAVYYCARGNSYFYALDYWGQGTSVTVSS

SEQ ID NO: 29 - protein, 119 aa, synthetic construct
  REGION 1..119: note = Heavy Chain of cofetuzumab IgG histidine scanning variant #17
QVQLVQSGPEVKKPGASVKVSCKASGYTFTDYAVHWVRQAPGKRLEWIGVISHYNDYTYNNQDFKGRVTMTRDTSASTAYMELSRLRSEDTAVYYCARGNSYFYALDYWGQGTSVTVSS

SEQ ID NO: 30 - protein, 119 aa, synthetic construct
  REGION 1..119: note = Heavy Chain of cofetuzumab IgG histidine scanning variant #18
QVQLVQSGPEVKKPGASVKVSCKASGYTFTDYAVHWVRQAPGKRLEWIGVISTHNDYTYNNQDFKGRVTMTRDTSASTAYMELSRLRSEDTAVYYCARGNSYFYALDYWGQGTSVTVSS

SEQ ID NO: 31 - protein, 119 aa, synthetic construct
  REGION 1..119: note = Heavy Chain of cofetuzumab IgG histidine scanning variant #19
QVQLVQSGPEVKKPGASVKVSCKASGYTFTDYAVHWVRQAPGKRLEWIGVISTYHDYTYNNQDFKGRVTMTRDTSASTAYMELSRLRSEDTAVYYCARGNSYFYALDYWGQGTSVTVSS

SEQ ID NO: 32 - protein, 119 aa, synthetic construct
  REGION 1..119: note = Heavy Chain of cofetuzumab IgG histidine scanning variant #20
QVQLVQSGPEVKKPGASVKVSCKASGYTFTDYAVHWVRQAPGKRLEWIGVISTYNHYTYNNQDFKGRVTMTRDTSASTAYMELSRLRSEDTAVYYCARGNSYFYALDYWGQGTSVTVSS

SEQ ID NO: 33 - protein, 119 aa, synthetic construct
  REGION 1..119: note = Heavy Chain of cofetuzumab IgG histidine scanning variant #21
QVQLVQSGPEVKKPGASVKVSCKASGYTFTDYAVHWVRQAPGKRLEWIGVISTYNDHTYNNQDFKGRVTMTRDTSASTAYMELSRLRSEDTAVYYCARGNSYFYALDYWGQGTSVTVSS

SEQ ID NO: 34 - protein, 119 aa, synthetic construct
  REGION 1..119: note = Heavy Chain of cofetuzumab IgG histidine scanning variant #22
QVQLVQSGPEVKKPGASVKVSCKASGYTFTDYAVHWVRQAPGKRLEWIGVISTYNDYHYNNQDFKGRVTMTRDTSASTAYMELSRLRSEDTAVYYCARGNSYFYALDYWGQGTSVTVSS

SEQ ID NO: 35 - protein, 119 aa, synthetic construct
  REGION 1..119: note = Heavy Chain of cofetuzumab IgG histidine scanning variant #23
QVQLVQSGPEVKKPGASVKVSCKASGYTFTDYAVHWVRQAPGKRLEWIGVISTYNDYTHNNQDFKGRVTMTRDTSASTAYMELSRLRSEDTAVYYCARGNSYFYALDYWGQGTSVTVSS

SEQ ID NO: 36 - protein, 119 aa, synthetic construct
  REGION 1..119: note = Heavy Chain of cofetuzumab IgG histidine scanning variant #24
QVQLVQSGPEVKKPGASVKVSCKASGYTFTDYAVHWVRQAPGKRLEWIGVISTYNDYTYHNQDFKGRVTMTRDTSASTAYMELSRLRSEDTAVYYCARGNSYFYALDYWGQGTSVTVSS

SEQ ID NO: 37 - protein, 119 aa, synthetic construct
  REGION 1..119: note = Heavy Chain of cofetuzumab IgG histidine scanning variant #25
QVQLVQSGPEVKKPGASVKVSCKASGYTFTDYAVHWVRQAPGKRLEWIGVISTYNDYTYNHQDFKGRVTMTRDTSASTAYMELSRLRSEDTAVYYCARGNSYFYALDYWGQGTSVTVSS

SEQ ID NO: 38 - protein, 119 aa, synthetic construct
  REGION 1..119: note = Heavy Chain of cofetuzumab IgG histidine scanning variant #26
QVQLVQSGPEVKKPGASVKVSCKASGYTFTDYAVHWVRQAPGKRLEWIGVISTYNDYTYNNHDFKGRVTMTRDTSASTAYMELSRLRSEDTAVYYCARGNSYFYALDYWGQGTSVTVSS

SEQ ID NO: 39 - protein, 119 aa, synthetic construct
  REGION 1..119: note = Heavy Chain of cofetuzumab IgG histidine scanning variant #27
QVQLVQSGPEVKKPGASVKVSCKASGYTFTDYAVHWVRQAPGKRLEWIGVISTYNDYTYNNQHFKGRVTMTRDTSASTAYMELSRLRSEDTAVYYCARGNSYFYALDYWGQGTSVTVSS

SEQ ID NO: 40 - protein, 119 aa, synthetic construct
  REGION 1..119: note = Heavy Chain of cofetuzumab IgG histidine scanning variant #28
QVQLVQSGPEVKKPGASVKVSCKASGYTFTDYAVHWVRQAPGKRLEWIGVISTYNDYTYNNQDHKGRVTMTRDTSASTAYMELSRLRSEDTAVYYCARGNSYFYALDYWGQGTSVTVSS

SEQ ID NO: 41 - protein, 119 aa, synthetic construct
  REGION 1..119: note = Heavy Chain of cofetuzumab IgG histidine scanning variant #29
QVQLVQSGPEVKKPGASVKVSCKASGYTFTDYAVHWVRQAPGKRLEWIGVISTYNDYTYNNQDFHGRVTMTRDTSASTAYMELSRLRSEDTAVYYCARGNSYFYALDYWGQGTSVTVSS

SEQ ID NO: 42 - protein, 119 aa, synthetic construct
  REGION 1..119: note = Heavy Chain of cofetuzumab IgG histidine scanning variant #30
QVQLVQSGPEVKKPGASVKVSCKASGYTFTDYAVHWVRQAPGKRLEWIGVISTYNDYTYNNQDFKHRVTMTRDTSASTAYMELSRLRSEDTAVYYCARGNSYFYALDYWGQGTSVTVSS

SEQ ID NO: 43 - protein, 119 aa, synthetic construct
  REGION 1..119: note = Heavy Chain of cofetuzumab IgG histidine scanning variant #31
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCHRGNSYFYALDYWGQGTSVTVSS

SEQ ID NO: 44 - protein, 119 aa, synthetic construct
  REGION 1..119: note = Heavy Chain of cofetuzumab IgG histidine scanning variant #32
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCAHGNSYFYALDYWGQGTSVTVSS

SEQ ID NO: 45 - protein, 119 aa, synthetic construct
  REGION 1..119: note = Heavy Chain of cofetuzumab IgG histidine scanning variant #33
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARHNSYFYALDYWGQGTSVTVSS

SEQ ID NO: 46 - protein, 119 aa, synthetic construct
  REGION 1..119: note = Heavy Chain of cofetuzumab IgG histidine scanning variant #34
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGHSYFYALDYWGQGTSVTVSS

SEQ ID NO: 47 - protein, 119 aa, synthetic construct
  REGION 1..119: note = Heavy Chain of cofetuzumab IgG histidine scanning variant #35
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNHYFYALDYWGQGTSVTVSS

SEQ ID NO: 48 - protein, 119 aa, synthetic construct
  REGION 1..119: note = Heavy Chain of cofetuzumab IgG histidine scanning variant #36
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSHFYALDYWGQGTSVTVSS

SEQ ID NO: 49 - protein, 119 aa, synthetic construct
  REGION 1..119: note = Heavy Chain of cofetuzumab IgG histidine scanning variant #37
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYHYALDYWGQGTSVTVSS

SEQ ID NO: 50 - protein, 119 aa, synthetic construct
  REGION 1..119: note = Heavy Chain of cofetuzumab IgG histidine scanning variant #38
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFHALDYWGQGTSVTVSS

SEQ ID NO: 51 - protein, 119 aa, synthetic construct
  REGION 1..119: note = Heavy Chain of cofetuzumab IgG histidine scanning variant #39
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFYHLDYWGQGTSVTVSS

SEQ ID NO: 52 - protein, 119 aa, synthetic construct
  REGION 1..119: note = Heavy Chain of cofetuzumab IgG histidine scanning variant #40
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFYAHDYWGQGTSVTVSS

SEQ ID NO: 53 - protein, 119 aa, synthetic construct
  REGION 1..119: note = Heavy Chain of cofetuzumab IgG histidine scanning variant #41
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFYALHYWGQGTSVTVSS

SEQ ID NO: 54 - protein, 119 aa, synthetic construct
  REGION 1..119: note = Heavy Chain of cofetuzumab IgG histidine scanning variant #42
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFYALDHWGQGTSVTVSS

SEQ ID NO: 55 - protein, 111 aa, synthetic construct
  REGION 1..111: note = Light Chain of cofetuzumab IgG histidine scanning variant #1
EIVLTQSPATLSLSPGERATLSCHASESVDSYGKSFMHWYQQKPGQAPRLLIYRASNLESGIPARFSGSGSGTDFTLTISSLEPEDFAVYYCQQSNEDPWTFGGGTKLEIK

SEQ ID NO: 56 - protein, 111 aa, synthetic construct
  REGION 1..111: note = Light Chain of cofetuzumab IgG histidine scanning variant #2
EIVLTQSPATLSLSPGERATLSCRHSESVDSYGKSFMHWYQQKPGQAPRLLIYRASNLESGIPARFSGSGSGTDFTLTISSLEPEDFAVYYCQQSNEDPWTFGGGTKLEIK

SEQ ID NO: 57 - protein, 111 aa, synthetic construct
  REGION 1..111: note = Light Chain of cofetuzumab IgG histidine scanning variant #3
EIVLTQSPATLSLSPGERATLSCRAHESVDSYGKSFMHWYQQKPGQAPRLLIYRASNLESGIPARFSGSGSGTDFTLTISSLEPEDFAVYYCQQSNEDPWTFGGGTKLEIK

SEQ ID NO: 58 - protein, 111 aa, synthetic construct
  REGION 1..111: note = Light Chain of cofetuzumab IgG histidine scanning variant #4
EIVLTQSPATLSLSPGERATLSCRASHSVDSYGKSFMHWYQQKPGQAPRLLIYRASNLESGIPARFSGSGSGTDFTLTISSLEPEDFAVYYCQQSNEDPWTFGGGTKLEIK

SEQ ID NO: 59 - protein, 111 aa, synthetic construct
  REGION 1..111: note = Light Chain of cofetuzumab IgG histidine scanning variant #5
EIVLTQSPATLSLSPGERATLSCRASEHVDSYGKSFMHWYQQKPGQAPRLLIYRASNLESGIPARFSGSGSGTDFTLTISSLEPEDFAVYYCQQSNEDPWTFGGGTKLEIK

SEQ ID NO: 60 - protein, 111 aa, synthetic construct
  REGION 1..111: note = Light Chain of cofetuzumab IgG histidine scanning variant #6
EIVLTQSPATLSLSPGERATLSCRASESHDSYGKSFMHWYQQKPGQAPRLLIYRASNLESGIPARFSGSGSGTDFTLTISSLEPEDFAVYYCQQSNEDPWTFGGGTKLEIK

SEQ ID NO: 61 - protein, 111 aa, synthetic construct
  REGION 1..111: note = Light Chain of cofetuzumab IgG histidine scanning variant #7
EIVLTQSPATLSLSPGERATLSCRASESVHSYGKSFMHWYQQKPGQAPRLLIYRASNLESGIPARFSGSGSGTDFTLTISSLEPEDFAVYYCQQSNEDPWTFGGGTKLEIK

SEQ ID NO: 62 - protein, 111 aa, synthetic construct
  REGION 1..111: note = Light Chain of cofetuzumab IgG histidine scanning variant #8
EIVLTQSPATLSLSPGERATLSCRASESVDHYGKSFMHWYQQKPGQAPRLLIYRASNLESGIPARFSGSGSGTDFTLTISSLEPEDFAVYYCQQSNEDPWTFGGGTKLEIK

SEQ ID NO: 63 - protein, 111 aa, synthetic construct
  REGION 1..111: note = Light Chain of cofetuzumab IgG histidine scanning variant #9
EIVLTQSPATLSLSPGERATLSCRASESVDSHGKSFMHWYQQKPGQAPRLLIYRASNLESGIPARFSGSGSGTDFTLTISSLEPEDFAVYYCQQSNEDPWTFGGGTKLEIK

SEQ ID NO: 64 - protein, 111 aa, synthetic construct
  REGION 1..111: note = Light Chain of cofetuzumab IgG histidine scanning variant #10
EIVLTQSPATLSLSPGERATLSCRASESVDSYHKSFMHWYQQKPGQAPRLLIYRASNLESGIPARFSGSGSGTDFTLTISSLEPEDFAVYYCQQSNEDPWTFGGGTKLEIK

SEQ ID NO: 65 - protein, 111 aa, synthetic construct
  REGION 1..111: note = Light Chain of cofetuzumab IgG histidine scanning variant #11
EIVLTQSPATLSLSPGERATLSCRASESVDSYGHSFMHWYQQKPGQAPRLLIYRASNLESGIPARFSGSGSGTDFTLTISSLEPEDFAVYYCQQSNEDPWTFGGGTKLEIK

SEQ ID NO: 66 - protein, 111 aa, synthetic construct
  REGION 1..111: note = Light Chain of cofetuzumab IgG histidine scanning variant #12
EIVLTQSPATLSLSPGERATLSCRASESVDSYGKHFMHWYQQKPGQAPRLLIYRASNLESGIPARFSGSGSGTDFTLTISSLEPEDFAVYYCQQSNEDPWTFGGGTKLEIK

SEQ ID NO: 67 - protein, 111 aa, synthetic construct
  REGION 1..111: note = Light Chain of cofetuzumab IgG histidine scanning variant #13
EIVLTQSPATLSLSPGERATLSCRASESVDSYGKSHMHWYQQKPGQAPRLLIYRASNLESGIPARFSGSGSGTDFTLTISSLEPEDFAVYYCQQSNEDPWTFGGGTKLEIK

SEQ ID NO: 68 - protein, 111 aa, synthetic construct
  REGION 1..111: note = Light Chain of cofetuzumab IgG histidine scanning variant #14
EIVLTQSPATLSLSPGERATLSCRASESVDSYGKSFHHWYQQKPGQAPRLLIYRASNLESGIPARFSGSGSGTDFTLTISSLEPEDFAVYYCQQSNEDPWTFGGGTKLEIK

SEQ ID NO: 69 - protein, 111 aa, synthetic construct
  REGION 1..111: note = Light Chain of cofetuzumab IgG histidine scanning variant #15
EIVLTQSPATLSLSPGERATLSCRASESVDSYGKSFMAWYQQKPGQAPRLLIYRASNLESGIPARFSGSGSGTDFTLTISSLEPEDFAVYYCQQSNEDPWTFGGGTKLEIK

SEQ ID NO: 70 - protein, 111 aa, synthetic construct
  REGION 1..111: note = Light Chain of cofetuzumab IgG histidine scanning variant #16
EIVLTQSPATLSLSPGERATLSCRASESVDSYGKSFMHWYQQKPGQAPRLLIYHASNLESGIPARFSGSGSGTDFTLTISSLEPEDFAVYYCQQSNEDPWTFGGGTKLEIK

SEQ ID NO: 71 - protein, 111 aa, synthetic construct
  REGION 1..111: note = Light Chain of cofetuzumab IgG histidine scanning variant #17
EIVLTQSPATLSLSPGERATLSCRASESVDSYGKSFMHWYQQKPGQAPRLLIYRHSNLESGIPARFSGSGSGTDFTLTISSLEPEDFAVYYCQQSNEDPWTFGGGTKLEIK

SEQ ID NO: 72 - protein, 111 aa, synthetic construct
  REGION 1..111: note = Light Chain of cofetuzumab IgG histidine scanning variant #18
EIVLTQSPATLSLSPGERATLSCRASESVDSYGKSFMHWYQQKPGQAPRLLIYRAHNLESGIPARFSGSGSGTDFTLTISSLEPEDFAVYYCQQSNEDPWTFGGGTKLEIK

SEQ ID NO: 73 - protein, 111 aa, synthetic construct
  REGION 1..111: note = Light Chain of cofetuzumab IgG histidine scanning variant #19
EIVLTQSPATLSLSPGERATLSCRASESVDSYGKSFMHWYQQKPGQAPRLLIYRASHLESGIPARFSGSGSGTDFTLTISSLEPEDFAVYYCQQSNEDPWTFGGGTKLEIK

SEQ ID NO: 74 - protein, 111 aa, synthetic construct
  REGION 1..111: note = Light Chain of cofetuzumab IgG histidine scanning variant #20
EIVLTQSPATLSLSPGERATLSCRASESVDSYGKSFMHWYQQKPGQAPRLLIYRASNHESGIPARFSGSGSGTDFTLTISSLEPEDFAVYYCQQSNEDPWTFGGGTKLEIK

SEQ ID NO: 75 - protein, 111 aa, synthetic construct
  REGION 1..111: note = Light Chain of cofetuzumab IgG histidine scanning variant #21
EIVLTQSPATLSLSPGERATLSCRASESVDSYGKSFMHWYQQKPGQAPRLLIYRASNLHSGIPARFSGSGSGTDFTLTISSLEPEDFAVYYCQQSNEDPWTFGGGTKLEIK

SEQ ID NO: 76 - protein, 111 aa, synthetic construct
  REGION 1..111: note = Light Chain of cofetuzumab IgG histidine scanning variant #22
EIVLTQSPATLSLSPGERATLSCRASESVDSYGKSFMHWYQQKPGQAPRLLIYRASNLEHGIPARFSGSGSGTDFTLTISSLEPEDFAVYYCQQSNEDPWTFGGGTKLEIK

SEQ ID NO: 77 - protein, 111 aa, synthetic construct
  REGION 1..111: note = Light Chain of cofetuzumab IgG histidine scanning variant #23
EIVLTQSPATLSLSPGERATLSCRASESVDSYGKSFMHWYQQKPGQAPRLLIYRASNLESGIPARFSGSGSGTDFTLTISSLEPEDFAVYYCHQSNEDPWTFGGGTKLEIK

SEQ ID NO: 78 - protein, 111 aa, synthetic construct
  REGION 1..111: note = Light Chain of cofetuzumab IgG histidine scanning variant #24
EIVLTQSPATLSLSPGERATLSCRASESVDSYGKSFMHWYQQKPGQAPRLLIYRASNLESGIPARFSGSGSGTDFTLTISSLEPEDFAVYYCQHSNEDPWTFGGGTKLEIK

SEQ ID NO: 79 - protein, 111 aa, synthetic construct
  REGION 1..111: note = Light Chain of cofetuzumab IgG histidine scanning variant #25
EIVLTQSPATLSLSPGERATLSCRASESVDSYGKSFMHWYQQKPGQAPRLLIYRASNLESGIPARFSGSGSGTDFTLTISSLEPEDFAVYYCQQHNEDPWTFGGGTKLEIK

SEQ ID NO: 80 - protein, 111 aa, synthetic construct
  REGION 1..111: note = Light Chain of cofetuzumab IgG histidine scanning variant #26
EIVLTQSPATLSLSPGERATLSCRASESVDSYGKSFMHWYQQKPGQAPRLLIYRASNLESGIPARFSGSGSGTDFTLTISSLEPEDFAVYYCQQSHEDPWTFGGGTKLEIK

SEQ ID NO: 81 - protein, 111 aa, synthetic construct
  REGION 1..111: note = Light Chain of cofetuzumab IgG histidine scanning variant #27
EIVLTQSPATLSLSPGERATLSCRASESVDSYGKSFMHWYQQKPGQAPRLLIYRASNLESGIPARFSGSGSGTDFTLTISSLEPEDFAVYYCQQSNHDPWTFGGGTKLEIK

SEQ ID NO: 82 - protein, 111 aa, synthetic construct
  REGION 1..111: note = Light Chain of cofetuzumab IgG histidine scanning variant #28
EIVLTQSPATLSLSPGERATLSCRASESVDSYGKSFMHWYQQKPGQAPRLLIYRASNLESGIPARFSGSGSGTDFTLTISSLEPEDFAVYYCQQSNEHPWTFGGGTKLEIK

SEQ ID NO: 83 - protein, 111 aa, synthetic construct
  REGION 1..111: note = Light Chain of cofetuzumab IgG histidine scanning variant #29
EIVLTQSPATLSLSPGERATLSCRASESVDSYGKSFMHWYQQKPGQAPRLLIYRASNLESGIPARFSGSGSGTDFTLTISSLEPEDFAVYYCQQSNEDHWTFGGGTKLEIK

SEQ ID NO: 84 - protein, 111 aa, synthetic construct
  REGION 1..111: note = Light Chain of cofetuzumab IgG histidine scanning variant #30
EIVLTQSPATLSLSPGERATLSCRASESVDSYGKSFMHWYQQKPGQAPRLLIYRASNLESGIPARFSGSGSGTDFTLTISSLEPEDFAVYYCQQSNEDPHTFGGGTKLEIK

SEQ ID NO: 85 - protein, 111 aa, synthetic construct
  REGION 1..111: note = Light Chain of cofetuzumab IgG histidine scanning variant #31
EIVLTQSPATLSLSPGERATLSCRASESVDSYGKSFMHWYQQKPGQAPRLLIYRASNLESGIPARFSGSGSGTDFTLTISSLEPEDFAVYYCQQSNEDPWHFGGGTKLEIK

SEQ ID NO: 86 - protein, 119 aa, synthetic construct
  REGION 1..119: note = Heavy Chain Combinations of cofetuzumab IgG histidine scanning variant #1
QVQLVQSGPEVKKPGASVKVSCKASGHTHTDYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 87 - protein, 119 aa, synthetic construct
  REGION 1..119: note = Heavy Chain Combinations of cofetuzumab IgG histidine scanning variant #2
QVQLVQSGPEVKKPGASVKVSCKASGHTFTHYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 88 - protein, 119 aa, synthetic construct
  REGION 1..119: note = Heavy Chain Combinations of cofetuzumab IgG histidine scanning variant #3
QVQLVQSGPEVKKPGASVKVSCKASGHTFTDYAHHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 89 - protein, 119 aa, synthetic construct
  REGION 1..119: note = Heavy Chain Combinations of cofetuzumab IgG histidine scanning variant #4
QVQLVQSGPEVKKPGASVKVSCKASGHTFTDYAVHWVRQAPGKRLEWIGVISHYNDYTYNNQDFKGRVTMTRDTSASTAYMELSRLRSEDTAVYYCARGNSYFYALDYWGQGTSVTVSS

SEQ ID NO: 90 - protein, 119 aa, synthetic construct
  REGION 1..119: note = Heavy Chain Combinations of cofetuzumab IgG histidine scanning variant #5
QVQLVQSGPEVKKPGASVKVSCKASGYTHTHYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 91 - protein, 119 aa, synthetic construct
  REGION 1..119: note = Heavy Chain Combinations of cofetuzumab IgG histidine scanning variant #6
QVQLVQSGPEVKKPGASVKVSCKASGYTHTDYAHHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 92 - protein, 119 aa, synthetic construct
  REGION 1..119: note = Heavy Chain Combinations of cofetuzumab IgG histidine scanning variant #7
QVQLVQSGPEVKKPGASVKVSCKASGYTHTDYAVHWVRQAPGKRLEWIGVISHYNDYTYNNQDFKGRVTMTRDTSASTAYMELSRLRSEDTAVYYCARGNSYFYALDYWGQGTSVTVSS

SEQ ID NO: 93 - protein, 119 aa, synthetic construct
  REGION 1..119: note = Heavy Chain Combinations of cofetuzumab IgG histidine scanning variant #8
QVQLVQSGPEVKKPGASVKVSCKASGYTFTHYAHHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 94 - protein, 119 aa, synthetic construct
  REGION 1..119: note = Heavy Chain Combinations of cofetuzumab IgG histidine scanning variant #9
QVQLVQSGPEVKKPGASVKVSCKASGYTFTHYAVHWVRQAPGKRLEWIGVISHYNDYTYNNQDFKGRVTMTRDTSASTAYMELSRLRSEDTAVYYCARGNSYFYALDYWGQGTSVTVSS

SEQ ID NO: 95 - protein, 119 aa, synthetic construct
  REGION 1..119: note = Heavy Chain Combinations of cofetuzumab IgG histidine scanning variant #10
QVQLVQSGPEVKKPGASVKVSCKASGYTFTDYAHHWVRQAPGKRLEWIGVISHYNDYTYNNQDFKGRVTMTRDTSASTAYMELSRLRSEDTAVYYCARGNSYFYALDYWGQGTSVTVSS

SEQ ID NO: 96 - protein, 119 aa, synthetic construct
  REGION 1..119: note = Heavy Chain Combinations of cofetuzumab IgG histidine scanning variant #11
QVQLVQSGPEVKKPGASVKVSCKASGHTHTHYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 97 - protein, 119 aa, synthetic construct
  REGION 1..119: note = Heavy Chain Combinations of cofetuzumab IgG histidine scanning variant #12
QVQLVQSGPEVKKPGASVKVSCKASGHTHTDYAHHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 98 - protein, 119 aa, synthetic construct
  REGION 1..119: note = Heavy Chain Combinations of cofetuzumab IgG histidine scanning variant #13
QVQLVQSGPEVKKPGASVKVSCKASGHTHTDYAVHWVRQAPGKRLEWIGVISHYNDYTYNNQDFKGRVTMTRDTSASTAYMELSRLRSEDTAVYYCARGNSYFYALDYWGQGTSVTVSS

SEQ ID NO: 99 - protein, 119 aa, synthetic construct
  REGION 1..119: note = Heavy Chain Combinations of cofetuzumab IgG histidine scanning variant #14
QVQLVQSGPEVKKPGASVKVSCKASGHTFTHYAHHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 100 - protein, 119 aa, synthetic construct
  REGION 1..119: note = Heavy Chain Combinations of cofetuzumab IgG histidine scanning variant #15
QVQLVQSGPEVKKPGASVKVSCKASGHTFTHYAVHWVRQAPGKRLEWIGVISHYNDYTYNNQDFKGRVTMTRDTSASTAYMELSRLRSEDTAVYYCARGNSYFYALDYWGQGTSVTVSS

SEQ ID NO: 101 - protein, 119 aa, synthetic construct
  REGION 1..119: note = Heavy Chain Combinations of cofetuzumab IgG histidine scanning variant #16
QVQLVQSGPEVKKPGASVKVSCKASGHTFTDYAHHWVRQAPGKRLEWIGVISHYNDYTYNNQDFKGRVTMTRDTSASTAYMELSRLRSEDTAVYYCARGNSYFYALDYWGQGTSVTVSS

SEQ ID NO: 102 - protein, 119 aa, synthetic construct
  REGION 1..119: note = Heavy Chain Combinations of cofetuzumab IgG histidine scanning variant #17
QVQLVQSGPEVKKPGASVKVSCKASGYTHTHYAHHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 103 - protein, 119 aa, synthetic construct
  REGION 1..119: note = Heavy Chain Combinations of cofetuzumab IgG histidine scanning variant #18
QVQLVQSGPEVKKPGASVKVSCKASGYTHTHYAVHWVRQAPGKRLEWIGVISHYNDYTYNNQDFKGRVTMTRDTSASTAYMELSRLRSEDTAVYYCARGNSYFYALDYWGQGTSVTVSS

SEQ ID NO: 104 - protein, 119 aa, synthetic construct
  REGION 1..119: note = Heavy Chain Combinations of cofetuzumab IgG histidine scanning variant #19
QVQLVQSGPEVKKPGASVKVSCKASGYTHTDYAHHWVRQAPGKRLEWIGVISHYNDYTYNNQDFKGRVTMTRDTSASTAYMELSRLRSEDTAVYYCARGNSYFYALDYWGQGTSVTVSS

SEQ ID NO: 105 - protein, 119 aa, synthetic construct
  REGION 1..119: note = Heavy Chain Combinations of cofetuzumab IgG histidine scanning variant #20
QVQLVQSGPEVKKPGASVKVSCKASGYTFTHYAHHWVRQAPGKRLEWIGVISHYNDYTYNNQDFKGRVTMTRDTSASTAYMELSRLRSEDTAVYYCARGNSYFYALDYWGQGTSVTVSS

SEQ ID NO: 106 - protein, 111 aa, synthetic construct
  REGION 1..111: note = Light Chain Combinations of cofetuzumab IgG histidine scanning variant #1
EIVLTQSPATLSLSPGERATLSCRAHESVDHYGKSFMHWYQQKPGQAPRLLIYRASNLESGIPARFSGSGSGTDFTLTISSLEPEDFAVYYCQQSNEDPWTFGGGTKLEIK

SEQ ID NO: 107 - protein, 111 aa, synthetic construct
  REGION 1..111: note = Light Chain Combinations of cofetuzumab IgG histidine scanning variant #2
EIVLTQSPATLSLSPGERATLSCRAHESVDSYGKSFMAWYQQKPGQAPRLLIYRASNLESGIPARFSGSGSGTDFTLTISSLEPEDFAVYYCQQSNEDPWTFGGGTKLEIK

SEQ ID NO: 108 - protein, 111 aa, synthetic construct
  REGION 1..111: note = Light Chain Combinations of cofetuzumab IgG histidine scanning variant #3
EIVLTQSPATLSLSPGERATLSCRAHESVDSYGKSFMHWYQQKPGQAPRLLIYRASNLEHGIPARFSGSGSGTDFTLTISSLEPEDFAVYYCQQSNEDPWTFGGGTKLEIK

SEQ ID NO: 109 - protein, 111 aa, synthetic construct
  REGION 1..111: note = Light Chain Combinations of cofetuzumab IgG histidine scanning variant #4
EIVLTQSPATLSLSPGERATLSCRAHESVDSYGKSFMHWYQQKPGQAPRLLIYRASNLESGIPARFSGSGSGTDFTLTISSLEPEDFAVYYCHQSNEDPWTFGGGTKLEIK

SEQ ID NO: 110 - protein, 111 aa, synthetic construct
  REGION 1..111: note = Light Chain Combinations of cofetuzumab IgG histidine scanning variant #5
EIVLTQSPATLSLSPGERATLSCRASESVDHYGKSFMAWYQQKPGQAPRLLIYRASNLESGIPARFSGSGSGTDFTLTISSLEPEDFAVYYCQQSNEDPWTFGGGTKLEIK

SEQ ID NO: 111 - protein, 111 aa, synthetic construct
  REGION 1..111: note = Light Chain Combinations of cofetuzumab IgG histidine scanning variant #6
EIVLTQSPATLSLSPGERATLSCRASESVDHYGKSFMHWYQQKPGQAPRLLIYRASNLEHGIPARFSGSGSGTDFTLTISSLEPEDFAVYYCQQSNEDPWTFGGGTKLEIK

SEQ ID NO: 112 - protein, 111 aa, synthetic construct
  REGION 1..111: note = Light Chain Combinations of cofetuzumab IgG histidine scanning variant #7
EIVLTQSPATLSLSPGERATLSCRASESVDHYGKSFMHWYQQKPGQAPRLLIYRASNLESGIPARFSGSGSGTDFTLTISSLEPEDFAVYYCHQSNEDPWTFGGGTKLEIK

SEQ ID NO: 113 - protein, 111 aa, synthetic construct
  REGION 1..111: note = Light Chain Combinations of cofetuzumab IgG histidine scanning variant #8
EIVLTQSPATLSLSPGERATLSCRASESVDSYGKSFMAWYQQKPGQAPRLLIYRASNLEHGIPARFSGSGSGTDFTLTISSLEPEDFAVYYCQQSNEDPWTFGGGTKLEIK

SEQ ID NO: 114 - protein, 111 aa, synthetic construct
  REGION 1..111: note = Light Chain Combinations of cofetuzumab IgG histidine scanning variant #9
EIVLTQSPATLSLSPGERATLSCRASESVDSYGKSFMAWYQQKPGQAPRLLIYRASNLESGIPARFSGSGSGTDFTLTISSLEPEDFAVYYCHQSNEDPWTFGGGTKLEIK

SEQ ID NO: 115 - protein, 111 aa, synthetic construct
  REGION 1..111: note = Light Chain Combinations of cofetuzumab IgG histidine scanning variant #10
EIVLTQSPATLSLSPGERATLSCRASESVDSYGKSFMHWYQQKPGQAPRLLIYRASNLEHGIPARFSGSGSGTDFTLTISSLEPEDFAVYYCHQSNEDPWTFGGGTKLEIK

SEQ ID NO: 116 - protein, 111 aa, synthetic construct
  REGION 1..111: note = Light Chain Combinations of cofetuzumab IgG histidine scanning variant #11
EIVLTQSPATLSLSPGERATLSCRAHESVDHYGKSFMAWYQQKPGQAPRLLIYRASNLESGIPARFSGSGSGTDFTLTISSLEPEDFAVYYCQQSNEDPWTFGGGTKLEIK

SEQ ID NO: 117 - protein, 111 aa, synthetic construct
  REGION 1..111: note = Light Chain Combinations of cofetuzumab IgG histidine scanning variant #12
EIVLTQSPATLSLSPGERATLSCRAHESVDHYGKSFMHWYQQKPGQAPRLLIYRASNLEHGIPARFSGSGSGTDFTLTISSLEPEDFAVYYCQQSNEDPWTFGGGTKLEIK

SEQ ID NO: 118 - protein, 111 aa, synthetic construct
  REGION 1..111: note = Light Chain Combinations of cofetuzumab IgG histidine scanning variant #13
EIVLTQSPATLSLSPGERATLSCRAHESVDHYGKSFMHWYQQKPGQAPRLLIYRASNLESGIPARFSGSGSGTDFTLTISSLEPEDFAVYYCHQSNEDPWTFGGGTKLEIK

SEQ ID NO: 119 - protein, 111 aa, synthetic construct
  REGION 1..111: note = Light Chain Combinations of cofetuzumab IgG histidine scanning variant #14
EIVLTQSPATLSLSPGERATLSCRAHESVDSYGKSFMAWYQQKPGQAPRLLIYRASNLEHGIPARFSGSGSGTDFTLTISSLEPEDFAVYYCQQSNEDPWTFGGGTKLEIK

SEQ ID NO: 120 - protein, 111 aa, synthetic construct
  REGION 1..111: note = Light Chain Combinations of cofetuzumab IgG histidine scanning variant #15
EIVLTQSPATLSLSPGERATLSCRAHESVDSYGKSFMAWYQQKPGQAPRLLIYRASNLESGIPARFSGSGSGTDFTLTISSLEPEDFAVYYCHQSNEDPWTFGGGTKLEIK

SEQ ID NO: 121 - protein, 111 aa, synthetic construct
  REGION 1..111: note = Light Chain Combinations of cofetuzumab IgG histidine scanning variant #16
EIVLTQSPATLSLSPGERATLSCRAHESVDSYGKSFMHWYQQKPGQAPRLLIYRASNLEHGIPARFSGSGSGTDFTLTISSLEPEDFAVYYCHQSNEDPWTFGGGTKLEIK

SEQ ID NO: 122 - protein, 111 aa, synthetic construct
  REGION 1..111: note = Light Chain Combinations of cofetuzumab IgG histidine scanning variant #17
EIVLTQSPATLSLSPGERATLSCRASESVDHYGKSFMAWYQQKPGQAPRLLIYRASNLEHGIPARFSGSGSGTDFTLTISSLEPEDFAVYYCQQSNEDPWTFGGGTKLEIK

SEQ ID NO: 123 - protein, 111 aa, synthetic construct
  REGION 1..111: note = Light Chain Combinations of cofetuzumab IgG histidine scanning variant #18
EIVLTQSPATLSLSPGERATLSCRASESVDHYGKSFMAWYQQKPGQAPRLLIYRASNLESGIPARFSGSGSGTDFTLTISSLEPEDFAVYYCHQSNEDPWTFGGGTKLEIK

SEQ ID NO: 124 - protein, 111 aa, synthetic construct
  REGION 1..111: note = Light Chain Combinations of cofetuzumab IgG histidine scanning variant #19
EIVLTQSPATLSLSPGERATLSCRASESVDHYGKSFMHWYQQKPGQAPRLLIYRASNLEHGIPARFSGSGSGTDFTLTISSLEPEDFAVYYCHQSNEDPWTFGGGTKLEIK

SEQ ID NO: 125 - protein, 111 aa, synthetic construct
  REGION 1..111: note = Light Chain Combinations of cofetuzumab IgG histidine scanning variant #20
EIVLTQSPATLSLSPGERATLSCRASESVDSYGKSFMAWYQQKPGQAPRLLIYRASNLEHGIPARFSGSGSGTDFTLTISSLEPEDFAVYYCHQSNEDPWTFGGGTKLEIK

SEQ ID NO: 126 - protein, 126 aa, synthetic construct
  REGION 1..126: note = Heavy Chain of 7C8 IgG
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSS

SEQ ID NO: 127 - protein, 108 aa, synthetic construct
  REGION 1..108: note = Light Chain of 7C8 IgG
EIVLTQSPATLSLSPGERATLSCRASQSVSIYLAWYQQKPGQAPRLLIYDASNRATGIPARFSGSGSGTDFTLTISSLEPEDFAVYYCQQRSNWPPFTFGPGTKVDIK

SEQ ID NO: 128 - protein, 10 aa, synthetic construct
  REGION 1..10: note = Heavy Chain CDR1 of 7C8 IgG
GFTFSSYGMH

SEQ ID NO: 129 - protein, 17 aa, synthetic construct
  REGION 1..17: note = Heavy Chain CDR2 of 7C8 IgG
VIWDDGSNKYYVDSVKG

SEQ ID NO: 130 - protein, 19 aa, synthetic construct
  REGION 1..19: note = Heavy Chain CDR3 of 7C8 IgG
ARDDYYGSGSFNSYYGTDV

SEQ ID NO: 131 - protein, 11 aa, synthetic construct
  REGION 1..11: note = Light Chain CDR1 of 7C8 IgG
RASQSVSIYLA

SEQ ID NO: 132 - protein, 7 aa, synthetic construct
  REGION 1..7: note = Light Chain CDR2 of 7C8 IgG
DASNRAT

SEQ ID NO: 133 - protein, 10 aa, synthetic construct
  REGION 1..10: note = Light Chain CDR3 of 7C8 IgG
QQRSNWPPFT

SEQ ID NO: 134 - protein, 126 aa, synthetic construct
  REGION 1..126: note = Heavy Chain of 7C8 IgG histidine scanning variant #1
QVQLVESGGGVVQPGRSLRLSCAASHFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSS

SEQ ID NO: 135 - protein, 126 aa, synthetic construct
  REGION 1..126: note = Heavy Chain of 7C8 IgG histidine scanning variant #2
QVQLVESGGGVVQPGRSLRLSCAASGHTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSS

SEQ ID NO: 136 - protein, 126 aa, synthetic construct
  REGION 1..126: note = Heavy Chain of 7C8 IgG histidine scanning variant #3
QVQLVESGGGVVQPGRSLRLSCAASGFHFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSS

SEQ ID NO: 137 - protein, 126 aa, synthetic construct
  REGION 1..126: note = Heavy Chain of 7C8 IgG histidine scanning variant #4
QVQLVESGGGVVQPGRSLRLSCAASGFTHSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSS

SEQ ID NO: 138 - protein, 126 aa, synthetic construct
  REGION 1..126: note = Heavy Chain of 7C8 IgG histidine scanning variant #5
QVQLVESGGGVVQPGRSLRLSCAASGFTFHSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSS

SEQ ID NO: 139 - protein, 126 aa, synthetic construct
  REGION 1..126: note = Heavy Chain of 7C8 IgG histidine scanning variant #6
QVQLVESGGGVVQPGRSLRLSCAASGFTFSHYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSS

SEQ ID NO: 140 - protein, 126 aa, synthetic construct
  REGION 1..126: note = Heavy Chain of 7C8 IgG histidine scanning variant #7
QVQLVESGGGVVQPGRSLRLSCAASGFTFSSHGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSS

SEQ ID NO: 141 - protein, 126 aa, synthetic construct
  REGION 1..126: note = Heavy Chain of 7C8 IgG histidine scanning variant #8
QVQLVESGGGVVQPGRSLRLSCAASGFTFSSYHMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSS

SEQ ID NO: 142 - protein, 126 aa, synthetic construct
  REGION 1..126: note = Heavy Chain of 7C8 IgG histidine scanning variant #9
QVQLVESGGGVVQPGRSLRLSCAASGFTFSSYGHHWVRQAPGKGLEWVAVIWDDGSNKYYVDSVKGRFTISRDNSKNTLYLQMNSLRAEDTAVYYCARDDYYGSGSFNSYYGTDVWGQGTTVTVSS

SEQ ID NO: 143 - protein, 126 aa, synthetic construct
  REGION 1..126: note = Heavy Chain of 7C8 IgG histidine scanning variant #10
QVQLVESGGGVVQPGRSLRLSCAASGFTFSSYGMAWVRQAPGKGLEWVAVIWDDGSNKYYVDSVKGRFTISRDNSKNTLYLQMNSLRAEDTAVYYCARDDYYGSGSFNSYYGTDVWGQGTTVTVSS

SEQ ID NO: 144 - protein, 126 aa, synthetic construct
  REGION 1..126: note = Heavy Chain of 7C8 IgG histidine scanning variant #11
QVQLVESGGGVVQPGRSLRLSCAASGFTFSSYGMHWVRQAPGKGLEWVAHIWDDGSNKYYVDSVKGRFTISRDNSKNTLYLQMNSLRAEDTAVYYCARDDYYGSGSFNSYYGTDVWGQGTTVTVSS

SEQ ID NO: 145 - protein, 126 aa, synthetic construct
  REGION 1..126: note = Heavy Chain of 7C8 IgG histidine scanning variant #12
QVQLVESGGGVVQPGRSLRLSCAASGFTFSSYGMHWVRQAPGKGLEWVAVHWDDGSNKYYVDSVKGRFTISRDNSKNTLYLQMNSLRAEDTAVYYCARDDYYGSGSFNSYYGTDVWGQGTTVTVSS

SEQ ID NO: 146 - protein, 126 aa, synthetic construct
  REGION 1..126: note = Heavy Chain of 7C8 IgG histidine scanning variant #13
QVQLVESGGGVVQPGRSLRLSCAASGFTFSSYGMHWVRQAPGKGLEWVAVIHDDGSNKYYVDSVKGRFTISRDNSKNTLYLQMNSLRAEDTAVYYCARDDYYGSGSFNSYYGTDVWGQGTTVTVSS

SEQ ID NO: 147 - protein, 126 aa, synthetic construct
  REGION 1..126: note = Heavy Chain of 7C8 IgG histidine scanning variant #14
QVQLVESGGGVVQPGRSLRLSCAASGFTFSSYGMHWVRQAPGKGLEWVAVIWHDGSNKYYVDSVKGRFTISRDNSKNTLYLQMNSLRAEDTAVYYCARDDYYGSGSFNSYYGTDVWGQGTTVTVSS

SEQ ID NO: 148 - protein, 126 aa, synthetic construct
  REGION 1..126: note = Heavy Chain of 7C8 IgG histidine scanning variant #15
QVQLVESGGGVVQPGRSLRLSCAASGFTFSSYGMHWVRQAPGKGLEWVAVIWDHGSNKYYVDSVKGRFTISRDNSKNTLYLQMNSLRAEDTAVYYCARDDYYGSGSFNSYYGTDVWGQGTTVTVSS

SEQ ID NO: 149 - protein, 126 aa, synthetic construct
  REGION 1..126: note = Heavy Chain of 7C8 IgG histidine scanning variant #16
QVQLVESGGGVVQPGRSLRLSCAASGFTFSSYGMHWVRQAPGKGLEWVAVIWDDHSNKYYVDSVKGRFTISRDNSKNTLYLQMNSLRAEDTAVYYCARDDYYGSGSFNSYYGTDVWGQGTTVTVSS

SEQ ID NO: 150 - protein, 126 aa, synthetic construct
  REGION 1..126: note = Heavy Chain of 7C8 IgG histidine scanning variant #17
QVQLVESGGGVVQPGRSLRLSCAASGFTFSSYGMHWVRQAPGKGLEWVAVIWDDGHNKYYVDSVKGRFTISRDNSKNTLYLQMNSLRAEDTAVYYCARDDYYGSGSFNSYYGTDVWGQGTTVTVSS

SEQ ID NO: 151 - protein, 126 aa, synthetic construct
  REGION 1..126: note = Heavy Chain of 7C8 IgG histidine scanning variant #18
QVQLVESGGGVVQPGRSLRLSCAASGFTFSSYGMHWVRQAPGKGLEWVAVIWDDGSHKYYVDSVKGRFTISRDNSKNTLYLQMNSLRAEDTAVYYCARDDYYGSGSFNSYYGTDVWGQGTTVTVSS

SEQ ID NO: 152 - protein, 126 aa, synthetic construct
  REGION 1..126: note = Heavy Chain of 7C8 IgG histidine scanning variant #19
QVQLVESGGGVVQPGRSLRLSCAASGFTFSSYGMHWVRQAPGKGLEWVAVIWDDGSNHYYVDSVKGRFTISRDNSKNTLYLQMNSLRAEDTAVYYCARDDYYGSGSFNSYYGTDVWGQGTTVTVSS

SEQ ID NO: 153 - protein, 126 aa, synthetic construct
  REGION 1..126: note = Heavy Chain of 7C8 IgG histidine scanning variant #20
QVQLVESGGGVVQPGRSLRLSCAASGFTFSSYGMHWVRQAPGKGLEWVAVIWDDGSNKHYVDSVKGRFTISRDNSKNTLYLQMNSLRAEDTAVYYCARDDYYGSGSFNSYYGTDVWGQGTTVTVSS

SEQ ID NO: 154 - protein, 126 aa, synthetic construct
  REGION 1..126: note = Heavy Chain of 7C8 IgG histidine scanning variant #21
QVQLVESGGGVVQPGRSLRLSCAASGFTFSSYGMHWVRQAPGKGLEWVAVIWDDGSNKYHVDSVKGRFTISRDNSKNTLYLQMNSLRAEDTAVYYCARDDYYGSGSFNSYYGTDVWGQGTTVTVSS

SEQ ID NO: 155 - protein, 126 aa, synthetic construct
  REGION 1..126: note = Heavy Chain of 7C8 IgG histidine scanning variant #22
QVQLVESGGGVVQPGRSLRLSCAASGFTFSSYGMHWVRQAPGKGLEWVAVIWDDGSNKYYHDSVKGRFTISRDNSKNTLYLQMNSLRAEDTAVYYCARDDYYGSGSFNSYYGTDVWGQGTTVTVSS

SEQ ID NO: 156 - protein, 126 aa, synthetic construct
  REGION 1..126: note = Heavy Chain of 7C8 IgG histidine scanning variant #23
QVQLVESGGGVVQPGRSLRLSCAASGFTFSSYGMHWVRQAPGKGLEWVAVIWDDGSNKYYVHSVKGRFTISRDNSKNTLYLQMNSLRAEDTAVYYCARDDYYGSGSFNSYYGTDVWGQGTTVTVSS

SEQ ID NO: 157 - protein, 126 aa, synthetic construct
  REGION 1..126: note = Heavy Chain of 7C8 IgG histidine scanning variant #24
QVQLVESGGGVVQPGRSLRLSCAASGFTFSSYGMHWVRQAPGKGLEWVAVIWDDGSNKYYVDHVKGRFTISRDNSKNTLYLQMNSLRAEDTAVYYCARDDYYGSGSFNSYYGTDVWGQGTTVTVSS

SEQ ID NO: 158 - protein, 126 aa, synthetic construct
  REGION 1..126: note = Heavy Chain of 7C8 IgG histidine scanning variant #25
QVQLVESGGGVVQPGRSLRLSCAASGFTFSSYGMHWVRQAPGKGLEWVAVIWDDGSNKYYVDSHKGRFTISRDNSKNTLYLQMNSLRAEDTAVYYCARDDYYGSGSFNSYYGTDVWGQGTTVTVSS

SEQ ID NO: 159 - protein, 126 aa, synthetic construct
  REGION 1..126: note = Heavy Chain of 7C8 IgG histidine scanning variant #26
QVQLVESGGGVVQPGRSLRLSCAASGFTFSSYGMHWVRQAPGKGLEWVAVIWDDGSNKYYVDSVHGRFTISRDNSKNTLYLQMNSLRAEDTAVYYCARDDYYGSGSFNSYYGTDVWGQGTTVTVSS

SEQ ID NO: 160 - protein, 126 aa, synthetic construct
  REGION 1..126: note = Heavy Chain of 7C8 IgG histidine scanning variant #27
QVQLVESGGGVVQPGRSLRLSCAASGFTFSSYGMHWVRQAPGKGLEWVAVIWDDGSNKYYVDSVKHRFTISRDNSKNTLYLQMNSLRAEDTAVYYCARDDYYGSGSFNSYYGTDVWGQGTTVTVSS

SEQ ID NO: 161 - protein, 126 aa, synthetic construct
  REGION 1..126: note = Heavy Chain of 7C8 IgG histidine scanning variant #28
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCHRDDYYGSGSFNSYYGTDVWGQGTTVTVSS

SEQ ID NO: 162 - protein, 126 aa, synthetic construct
  REGION 1..126: note = Heavy Chain of 7C8 IgG histidine scanning variant #29
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCAHDDYYGSGSFNSYYGTDVWGQGTTVTVSS

SEQ ID NO: 163 - protein, 126 aa, synthetic construct
  REGION 1..126: note = Heavy Chain of 7C8 IgG histidine scanning variant #30
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARHDYYGSGSFNSYYGTDVWGQGTTVTVSS

SEQ ID NO: 164 - protein, 126 aa, synthetic construct
  REGION 1..126: note = Heavy Chain of 7C8 IgG histidine scanning variant #31
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDHYYGSGSFNSYYGTDVWGQGTTVTVSS

SEQ ID NO: 165 - protein, 126 aa, synthetic construct
  REGION 1..126: note = Heavy Chain of 7C8 IgG histidine scanning variant #32
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDHYGSGSFNSYYGTDVWGQGTTVTVSS

SEQ ID NO: 166 - protein, 126 aa, synthetic construct
  REGION 1..126: note = Heavy Chain of 7C8 IgG histidine scanning variant #33
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYHGSGSFNSYYGTDVWGQGTTVTVSS

SEQ ID NO: 167 - protein, 126 aa, synthetic construct
  REGION 1..126: note = Heavy Chain of 7C8 IgG histidine scanning variant #34
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYHSGSFNSYYGTDVWGQGTTVTVSS

SEQ ID NO: 168 - protein, 126 aa, synthetic construct
  REGION 1..126: note = Heavy Chain of 7C8 IgG histidine scanning variant #35
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGHGSFNSYYGTDVWGQGTTVTVSS

SEQ ID NO: 169 - protein, 126 aa, synthetic construct
  REGION 1..126: note = Heavy Chain of 7C8 IgG histidine scanning variant #36
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSHSFNSYYGTDVWGQGTTVTVSS

SEQ ID NO: 170 - protein, 126 aa, synthetic construct
  REGION 1..126: note = Heavy Chain of 7C8 IgG histidine scanning variant #37
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGHFNSYYGTDVWGQGTTVTVSS

SEQ ID NO: 171 - protein, 126 aa, synthetic construct
  REGION 1..126: note = Heavy Chain of 7C8 IgG histidine scanning variant #38
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSHNSYYGTDVWGQGTTVTVSS

SEQ ID NO: 172 - protein, 126 aa, synthetic construct
  REGION 1..126: note = Heavy Chain of 7C8 IgG histidine scanning variant #39
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFHSYYGTDVWGQGTTVTVSS

SEQ ID NO: 173 - protein, 126 aa, synthetic construct
  REGION 1..126: note = Heavy Chain of 7C8 IgG histidine scanning variant #40
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNHYYGTDVWGQGTTVTVSS

SEQ ID NO: 174 - protein, 126 aa, synthetic construct
  REGION 1..126: note = Heavy Chain of 7C8 IgG histidine scanning variant #41
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSHYGTDVWGQGTTVTVSS

SEQ ID NO: 175 - protein, 126 aa, synthetic construct
  REGION 1..126: note = Heavy Chain of 7C8 IgG histidine scanning variant #42
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYHGTDVWGQGTTVTVSS

SEQ ID NO: 176 - protein, 126 aa, synthetic construct
  REGION 1..126: note = Heavy Chain of 7C8 IgG histidine scanning variant #43
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYHTDVWGQGTTVTVSS

SEQ ID NO: 177 - protein, 126 aa, synthetic construct
  REGION 1..126: note = Heavy Chain of 7C8 IgG histidine scanning variant #44
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGHDVWGQGTTVTVSS

SEQ ID NO: 178 - protein, 126 aa, synthetic construct
  REGION 1..126: note = Heavy Chain of 7C8 IgG histidine scanning variant #45
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTHVWGQGTTVTVSS

SEQ ID NO: 179 - protein, 126 aa, synthetic construct
  REGION 1..126: note = Heavy Chain of 7C8 IgG histidine scanning variant #46
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDHWGQGTTVTVSS

SEQ ID NO: 180 - protein, 108 aa, synthetic construct
  REGION 1..108: note = Light Chain of 7C8 IgG histidine scanning variant #1
EIVLTQSPATLSLSPGERATLSCHASQSVSIYLAWYQQKPGQAPRLLIYDASNRATGIPARFSGSGSGTDFTLTISSLEPEDFAVYYCQQRSNWPPFTFGPGTKVDIK

SEQ ID NO: 181 - protein, 108 aa, synthetic construct
  REGION 1..108: note = Light Chain of 7C8 IgG histidine scanning variant #2
EIVLTQSPATLSLSPGERATLSCRHSQSVSIYLAWYQQKPGQAPRLLIYDASNRATGIPARFSGSGSGTDFTLTISSLEPEDFAVYYCQQRSNWPPFTFGPGTKVDIK

SEQ ID NO: 182 - protein, 108 aa, synthetic construct
  REGION 1..108: note = Light Chain of 7C8 IgG histidine scanning variant #3
EIVLTQSPATLSLSPGERATLSCRAHQSVSIYLAWYQQKPGQAPRLLIYDASNRATGIPARFSGSGSGTDFTLTISSLEPEDFAVYYCQQRSNWPPFTFGPGTKVDIK

SEQ ID NO: 183 - protein, 108 aa, synthetic construct
  REGION 1..108: note = Light Chain of 7C8 IgG histidine scanning variant #4
EIVLTQSPATLSLSPGERATLSCRASHSVSIYLAWYQQKPGQAPRLLIYDASNRATGIPARFSGSGSGTDFTLTISSLEPEDFAVYYCQQRSNWPPFTFGPGTKVDIK

SEQ ID NO: 184 - protein, 108 aa, synthetic construct
  REGION 1..108: note = Light Chain of 7C8 IgG histidine scanning variant #5
EIVLTQSPATLSLSPGERATLSCRASQHVSIYLAWYQQKPGQAPRLLIYDASNRATGIPARFSGSGSGTDFTLTISSLEPEDFAVYYCQQRSNWPPFTFGPGTKVDIK

SEQ ID NO: 185 - protein, 108 aa, synthetic construct
  REGION 1..108: note = Light Chain of 7C8 IgG histidine scanning variant #6
EIVLTQSPATLSLSPGERATLSCRASQSHSIYLAWYQQKPGQAPRLLIYDASNRATGIPARFSGSGSGTDFTLTISSLEPEDFAVYYCQQRSNWPPFTFGPGTKVDIK

SEQ ID NO: 186 - protein, 108 aa, synthetic construct
  REGION 1..108: note = Light Chain of 7C8 IgG histidine scanning variant #7
EIVLTQSPATLSLSPGERATLSCRASQSVHIYLAWYQQKPGQAPRLLIYDASNRATGIPARFSGSGSGTDFTLTISSLEPEDFAVYYCQQRSNWPPFTFGPGTKVDIK

SEQ ID NO: 187 - protein, 108 aa, synthetic construct
  REGION 1..108: note = Light Chain of 7C8 IgG histidine scanning variant #8
EIVLTQSPATLSLSPGERATLSCRASQSVSHYLAWYQQKPGQAPRLLIYDASNRATGIPARFSGSGSGTDFTLTISSLEPEDFAVYYCQQRSNWPPFTFGPGTKVDIK

SEQ ID NO: 188 - protein, 108 aa, synthetic construct
  REGION 1..108: note = Light Chain of 7C8 IgG histidine scanning variant #9
EIVLTQSPATLSLSPGERATLSCRASQSVSIHLAWYQQKPGQAPRLLIYDASNRATGIPARFSGSGSGTDFTLTISSLEPEDFAVYYCQQRSNWPPFTFGPGTKVDIK

SEQ ID NO: 189 - protein, 108 aa, synthetic construct
  REGION 1..108: note = Light Chain of 7C8 IgG histidine scanning variant #10
EIVLTQSPATLSLSPGERATLSCRASQSVSIYHAWYQQKPGQAPRLLIYDASNRATGIPARFSGSGSGTDFTLTISSLEPEDFAVYYCQQRSNWPPFTFGPGTKVDIK

SEQ ID NO: 190 - protein, 108 aa, synthetic construct
  REGION 1..108: note = Light Chain of 7C8 IgG histidine scanning variant #11
EIVLTQSPATLSLSPGERATLSCRASQSVSIYLHWYQQKPGQAPRLLIYDASNRATGIPARFSGSGSGTDFTLTISSLEPEDFAVYYCQQRSNWPPFTFGPGTKVDIK

SEQ ID NO: 191 - protein, 108 aa, synthetic construct
  REGION 1..108: note = Light Chain of 7C8 IgG histidine scanning variant #12
EIVLTQSPATLSLSPGERATLSCRASQSVSIYLAWYQQKPGQAPRLLIYHASNRATGIPARFSGSGSGTDFTLTISSLEPEDFAVYYCQQRSNWPPFTFGPGTKVDIK

SEQ ID NO: 192 - protein, 108 aa, synthetic construct
  REGION 1..108: note = Light Chain of 7C8 IgG histidine scanning variant #13
EIVLTQSPATLSLSPGERATLSCRASQSVSIYLAWYQQKPGQAPRLLIYDHSNRATGIPARFSGSGSGTDFTLTISSLEPEDFAVYYCQQRSNWPPFTFGPGTKVDIK

SEQ ID NO: 193 - protein, 108 aa, synthetic construct
  REGION 1..108: note = Light Chain of 7C8 IgG histidine scanning variant #14
EIVLTQSPATLSLSPGERATLSCRASQSVSIYLAWYQQKPGQAPRLLIYDAHNRATGIPARFSGSGSGTDFTLTISSLEPEDFAVYYCQQRSNWPPFTFGPGTKVDIK

SEQ ID NO: 194 - protein, 108 aa, synthetic construct
  REGION 1..108: note = Light Chain of 7C8 IgG histidine scanning variant #15
EIVLTQSPATLSLSPGERATLSCRASQSVSIYLAWYQQKPGQAPRLLIYDASHRATGIPARFSGSGSGTDFTLTISSLEPEDFAVYYCQQRSNWPPFTFGPGTKVDIK

SEQ ID NO: 195 - protein, 108 aa, synthetic construct
  REGION 1..108: note = Light Chain of 7C8 IgG histidine scanning variant #16
EIVLTQSPATLSLSPGERATLSCRASQSVSIYLAWYQQKPGQAPRLLIYDASNHATGIPARFSGSGSGTDFTLTISSLEPEDFAVYYCQQRSNWPPFTFGPGTKVDIK

SEQ ID NO: 196 - protein, 108 aa, synthetic construct
  REGION 1..108: note = Light Chain of 7C8 IgG histidine scanning variant #17
EIVLTQSPATLSLSPGERATLSCRASQSVSIYLAWYQQKPGQAPRLLIYDASNRHTGIPARFSGSGSGTDFTLTISSLEPEDFAVYYCQQRSNWPPFTFGPGTKVDIK

SEQ ID NO: 197 - protein, 108 aa, synthetic construct
  REGION 1..108: note = Light Chain of 7C8 IgG histidine scanning variant #18
EIVLTQSPATLSLSPGERATLSCRASQSVSIYLAWYQQKPGQAPRLLIYDASNRAHGIPARFSGSGSGTDFTLTISSLEPEDFAVYYCQQRSNWPPFTFGPGTKVDIK

SEQ ID NO: 198 - protein, 108 aa, synthetic construct
  REGION 1..108: note = Light Chain of 7C8 IgG histidine scanning variant #19
EIVLTQSPATLSLSPGERATLSCRASQSVSIYLAWYQQKPGQAPRLLIYDASNRATGIPARFSGSGSGTDFTLTISSLEPEDFAVYYCHQRSNWPPFTFGPGTKVDIK

SEQ ID NO: 199 - protein, 108 aa, synthetic construct
  REGION 1..108: note = Light Chain of 7C8 IgG histidine scanning variant #20
EIVLTQSPATLSLSPGERATLSCRASQSVSIYLAWYQQKPGQAPRLLIYDASNRATGIPARFSGSGSGTDFTLTISSLEPEDFAVYYCQHRSNWPPFTFGPGTKVDIK

SEQ ID NO: 200 - protein, 108 aa, synthetic construct
  REGION 1..108: note = Light Chain of 7C8 IgG histidine scanning variant #21
EIVLTQSPATLSLSPGERATLSCRASQSVSIYLAWYQQKPGQAPRLLIYDASNRATGIPARFSGSGSGTDFTLTISSLEPEDFAVYYCQQHSNWPPFTFGPGTKVDIK

SEQ ID NO: 201 - protein, 108 aa, synthetic construct
  REGION 1..108: note = Light Chain of 7C8 IgG histidine scanning variant #22
EIVLTQSPATLSLSPGERATLSCRASQSVSIYLAWYQQKPGQAPRLLIYDASNRATGIPARFSGSGSGTDFTLTISSLEPEDFAVYYCQQRHNWPPFTFGPGTKVDIK

SEQ ID NO: 202 - protein, 108 aa, synthetic construct
  REGION 1..108: note = Light Chain of 7C8 IgG histidine scanning variant #23
EIVLTQSPATLSLSPGERATLSCRASQSVSIYLAWYQQKPGQAPRLLIYDASNRATGIPARFSGSGSGTDFTLTISSLEPEDFAVYYCQQRSHWPPFTFGPGTKVDIK

SEQ ID NO: 203 - protein, 108 aa, synthetic construct
  REGION 1..108: note = Light Chain of 7C8 IgG histidine scanning variant #24
EIVLTQSPATLSLSPGERATLSCRASQSVSIYLAWYQQKPGQAPRLLIYDASNRATGIPARFSGSGSGTDFTLTISSLEPEDFAVYYCQQRSNHPPFTFGPGTKVDIK

SEQ ID NO: 204 - protein, 108 aa, synthetic construct
  REGION 1..108: note = Light Chain of 7C8 IgG histidine scanning variant #25
EIVLTQSPATLSLSPGERATLSCRASQSVSIYLAWYQQKPGQAPRLLIYDASNRATGIPARFSGSGSGTDFTLTISSLEPEDFAVYYCQQRSNWHPFTFGPGTKVDIK

SEQ ID NO: 205 - protein, 108 aa, synthetic construct
  REGION 1..108: note = Light Chain of 7C8 IgG histidine scanning variant #26
EIVLTQSPATLSLSPGERATLSCRASQSVSIYLAWYQQKPGQAPRLLIYDASNRATGIPARFSGSGSGTDFTLTISSLEPEDFAVYYCQQRSNWPHFTFGPGTKVDIK

SEQ ID NO: 206 - protein, 108 aa, synthetic construct
  REGION 1..108: note = Light Chain of 7C8 IgG histidine scanning variant #27
EIVLTQSPATLSLSPGERATLSCRASQSVSIYLAWYQQKPGQAPRLLIYDASNRATGIPARFSGSGSGTDFTLTISSLEPEDFAVYYCQQRSNWPPHTFGPGTKVDIK

SEQ ID NO: 207 - protein, 108 aa, synthetic construct
  REGION 1..108: note = Light Chain of 7C8 IgG histidine scanning variant #28
EIVLTQSPATLSLSPGERATLSCRASQSVSIYLAWYQQKPGQAPRLLIYDASNRATGIPARFSGSGSGTDFTLTISSLEPEDFAVYYCQQRSNWPPFHFGPGTKVDIK

SEQ ID NO: 208 - protein, 126 aa, synthetic construct
  REGION 1..126: note = Heavy Chain Combinations of 7C8 IgG histidine scanning variant #1
QVQLVESGGGVVQPGRSLRLSCAASGFTFSSYGMHWVRQAPGKGLEWVAVIWHDGHNKYYVDSVKGRFTISRDNSKNTLYLQMNSLRAEDTAVYYCARDDYYGSGSFNSYYGTDVWGQGTTVTVSS

SEQ ID NO: 209 - protein, 126 aa, synthetic construct
  REGION 1..126: note = Heavy Chain Combinations of 7C8 IgG histidine scanning variant #2
QVQLVESGGGVVQPGRSLRLSCAASGFTFSSYGMHWVRQAPGKGLEWVAVIWHDGSHKYYVDSVKGRFTISRDNSKNTLYLQMNSLRAEDTAVYYCARDDYYGSGSFNSYYGTDVWGQGTTVTVSS

SEQ ID NO: 210 - protein, 126 aa, synthetic construct
  REGION 1..126: note = Heavy Chain Combinations of 7C8 IgG histidine scanning variant #3
QVQLVESGGGVVQPGRSLRLSCAASGFTFSSYGMHWVRQAPGKGLEWVAVIWHDGSNKYYVDSVKGRFTISRDNSKNTLYLQMNSLRAEDTAVYYCARDDYHGSGSFNSYYGTDVWGQGTTVTVSS

SEQ ID NO: 211 - protein, 126 aa, synthetic construct
  REGION 1..126: note = Heavy Chain Combinations of 7C8 IgG histidine scanning variant #4
QVQLVESGGGVVQPGRSLRLSCAASGFTFSSYGMHWVRQAPGKGLEWVAVIWHDGSNKYYVDSVKGRFTISRDNSKNTLYLQMNSLRAEDTAVYYCARDDYYGSHSFNSYYGTDVWGQGTTVTVSS

SEQ ID NO: 212 - protein, 126 aa, synthetic construct
  REGION 1..126: note = Heavy Chain Combinations of 7C8 IgG histidine scanning variant #5
QVQLVESGGGVVQPGRSLRLSCAASGFTFSSYGMHWVRQAPGKGLEWVAVIWDDGHHKYYVDSVKGRFTISRDNSKNTLYLQMNSLRAEDTAVYYCARDDYYGSGSFNSYYGTDVWGQGTTVTVSS

SEQ ID NO: 213 - protein, 126 aa, synthetic construct
  REGION 1..126: note = Heavy Chain Combinations of 7C8 IgG histidine scanning variant #6
QVQLVESGGGVVQPGRSLRLSCAASGFTFSSYGMHWVRQAPGKGLEWVAVIWDDGHNKYYVDSVKGRFTISRDNSKNTLYLQMNSLRAEDTAVYYCARDDYHGSGSFNSYYGTDVWGQGTTVTVSS

SEQ ID NO: 214 - protein, 126 aa, synthetic construct
  REGION 1..126: note = Heavy Chain Combinations of 7C8 IgG histidine scanning variant #7
QVQLVESGGGVVQPGRSLRLSCAASGFTFSSYGMHWVRQAPGKGLEWVAVIWDDGHNKYYVDSVKGRFTISRDNSKNTLYLQMNSLRAEDTAVYYCARDDYYGSHSFNSYYGTDVWGQGTTVTVSS

SEQ ID NO: 215 - protein, 126 aa, synthetic construct
  REGION 1..126: note = Heavy Chain Combinations of 7C8 IgG histidine scanning variant #8
QVQLVESGGGVVQPGRSLRLSCAASGFTFSSYGMHWVRQAPGKGLEWVAVIWDDGSHKYYVDSVKGRFTISRDNSKNTLYLQMNSLRAEDTAVYYCARDDYHGSGSFNSYYGTDVWGQGTTVTVSS

SEQ ID NO: 216 - protein, 126 aa, synthetic construct
  REGION 1..126: note = Heavy Chain Combinations of 7C8 IgG histidine scanning variant #9
QVQLVESGGGVVQPGRSLRLSCAASGFTFSSYGMHWVRQAPGKGLEWVAVIWDDGSHKYYVDSVKGRFTISRDNSKNTLYLQMNSLRAEDTAVYYCARDDYYGSHSFNSYYGTDVWGQGTTVTVSS

SEQ ID NO: 217 - protein, 126 aa, synthetic construct
  REGION 1..126: note = Heavy Chain Combinations of 7C8 IgG histidine scanning variant #10
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYHGSHSFNSYYGTDVWGQGTTVTVSS

SEQ ID NO: 218 - protein, 126 aa, synthetic construct
  REGION 1..126: note = Heavy Chain Combinations of 7C8 IgG histidine scanning variant #11
QVQLVESGGGVVQPGRSLRLSCAASGFTFSSYGMHWVRQAPGKGLEWVAVIWHDGHHKYYVDSVKGRFTISRDNSKNTLYLQMNSLRAEDTAVYYCARDDYYGSGSFNSYYGTDVWGQGTTVTVSS

SEQ ID NO: 219 - protein, 126 aa, synthetic construct
  REGION 1..126: note = Heavy Chain Combinations of 7C8 IgG histidine scanning variant #12
QVQLVESGGGVVQPGRSLRLSCAASGFTFSSYGMHWVRQAPGKGLEWVAVIWHDGHNKYYVDSVKGRFTISRDNSKNTLYLQMNSLRAEDTAVYYCARDDYHGSGSFNSYYGTDVWGQGTTVTVSS

SEQ ID NO: 220 - protein, 126 aa, synthetic construct
  REGION 1..126: note = Heavy Chain Combinations of 7C8 IgG histidine scanning variant #13
QVQLVESGGGVVQPGRSLRLSCAASGFTFSSYGMHWVRQAPGKGLEWVAVIWHDGHNKYYVDSVKGRFTISRDNSKNTLYLQMNSLRAEDTAVYYCARDDYYGSHSFNSYYGTDVWGQGTTVTVSS

SEQ ID NO: 221 - protein, 126 aa, synthetic construct
  REGION 1..126: note = Heavy Chain Combinations of 7C8 IgG histidine scanning variant #14
QVQLVESGGGVVQPGRSLRLSCAASGFTFSSYGMHWVRQAPGKGLEWVAVIWHDGSHKYYVDSVKGRFTISRDNSKNTLYLQMNSLRAEDTAVYYCARDDYHGSGSFNSYYGTDVWGQGTTVTVSS

SEQ ID NO: 222 - protein, 126 aa, synthetic construct
  REGION 1..126: note = Heavy Chain Combinations of 7C8 IgG histidine scanning variant #15
QVQLVESGGGVVQPGRSLRLSCAASGFTFSSYGMHWVRQAPGKGLEWVAVIWHDGSHKYYVDSVKGRFTISRDNSKNTLYLQMNSLRAEDTAVYYCARDDYYGSHSFNSYYGTDVWGQGTTVTVSS

SEQ ID NO: 223 - protein, 126 aa, synthetic construct
  REGION 1..126: note = Heavy Chain Combinations of 7C8 IgG histidine scanning variant #16
QVQLVESGGGVVQPGRSLRLSCAASGFTFSSYGMHWVRQAPGKGLEWVAVIWHDGSNKYYVDSVKGRFTISRDNSKNTLYLQMNSLRAEDTAVYYCARDDYHGSHSFNSYYGTDVWGQGTTVTVSS

SEQ ID NO: 224 - protein, 126 aa, synthetic construct
  REGION 1..126: note = Heavy Chain Combinations of 7C8 IgG histidine scanning variant #17
QVQLVESGGGVVQPGRSLRLSCAASGFTFSSYGMHWVRQAPGKGLEWVAVIWDDGHHKYYVDSVKGRFTISRDNSKNTLYLQMNSLRAEDTAVYYCARDDYHGSGSFNSYYGTDVWGQGTTVTVSS

SEQ ID NO: 225 - protein, 126 aa, synthetic construct
  REGION 1..126: note = Heavy Chain Combinations of 7C8 IgG histidine scanning variant #18
QVQLVESGGGVVQPGRSLRLSCAASGFTFSSYGMHWVRQAPGKGLEWVAVIWDDGHHKYYVDSVKGRFTISRDNSKNTLYLQMNSLRAEDTAVYYCARDDYYGSHSFNSYYGTDVWGQGTTVTVSS

SEQ ID NO: 226 - protein, 126 aa, synthetic construct
  REGION 1..126: note = Heavy Chain Combinations of 7C8 IgG histidine scanning variant #19
QVQLVESGGGVVQPGRSLRLSCAASGFTFSSYGMHWVRQAPGKGLEWVAVIWDDGHNKYYVDSVKGRFTISRDNSKNTLYLQMNSLRAEDTAVYYCARDDYHGSHSFNSYYGTDVWGQGTTVTVSS

SEQ ID NO: 227 - protein, 126 aa, synthetic construct
  REGION 1..126: note = Heavy Chain Combinations of 7C8 IgG histidine scanning variant #20
QVQLVESGGGVVQPGRSLRLSCAASGFTFSSYGMHWVRQAPGKGLEWVAVIWDDGSHKYYVDSVKGRFTISRDNSKNTLYLQMNSLRAEDTAVYYCARDDYHGSHSFNSYYGTDVWGQGTTVTVSS

SEQ ID NO: 228 - protein, 108 aa, synthetic construct
  REGION 1..108: note = Light Chain Combinations of 7C8 IgG histidine scanning variant #1
EIVLTQSPATLSLSPGERATLSCRASQHVSIYLAWYQQKPGQAPRLLIYDASNRATGIPARFSGSGSGTDFTLTISSLEPEDFAVYYCQHRSNWPPFTFGPGTKVDIK

SEQ ID NO: 229 - protein, 112 aa, synthetic construct
  REGION 1..112: note = Heavy Chain of 12C6 IgG
EVQLVQSGGGLVHPGGSLRLSCAGSGFTFSTYLMYWVRQAPGKTLEWVSAIGSGGDTYYADSVKGRFTISRDNAKNSLYLQMNSLRAEDMAVYYCARGLGYWGQGTLVTVSS

SEQ ID NO: 230 - protein, 109 aa, synthetic construct
  REGION 1..109: note = Light Chain of 12C6 IgG
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPMYTFGQGTKLEIK

SEQ ID NO: 231 - protein, 10 aa, synthetic construct
  REGION 1..10: note = Heavy Chain CDR1 of 12C6 IgG
GFTFSTYLMY

SEQ ID NO: 232 - protein, 16 aa, synthetic construct
  REGION 1..16: note = Heavy Chain CDR2 of 12C6 IgG
AIGSGGDTYYADSVKG

SEQ ID NO: 233 - protein, 6 aa, synthetic construct
  REGION 1..6: note = Heavy Chain CDR3 of 12C6 IgG
ARGLGY

SEQ ID NO: 234 - protein, 12 aa, synthetic construct
  REGION 1..12: note = Light Chain CDR1 of 12C6 IgG
RASQSVSSSYLA

SEQ ID NO: 235 - protein, 7 aa, synthetic construct
  REGION 1..7: note = Light Chain CDR2 of 12C6 IgG
GASSRAT

SEQ ID NO: 236 - protein, 10 aa, synthetic construct
  REGION 1..10: note = Light Chain CDR3 of 12C6 IgG
QQYGSSPMYT

SEQ ID NO: 237 - protein, 112 aa, synthetic construct
  REGION 1..112: note = Heavy Chain of 12C6 IgG histidine scanning variant #1
EVQLVQSGGGLVHPGGSLRLSCAGSHFTFSTYLMYWVRQAPGKTLEWVSAIGSGGDTYYADSVKGRFTISRDNAKNSLYLQMNSLRAEDMAVYYCARGLGYWGQGTLVTVSS

SEQ ID NO: 238 - protein, 112 aa, synthetic construct
  REGION 1..112: note = Heavy Chain of 12C6 IgG histidine scanning variant #2
EVQLVQSGGGLVHPGGSLRLSCAGSGHTFSTYLMYWVRQAPGKTLEWVSAIGSGGDTYYADSVKGRFTISRDNAKNSLYLQMNSLRAEDMAVYYCARGLGYWGQGTLVTVSS

SEQ ID NO: 239 - protein, 112 aa, synthetic construct
  REGION 1..112: note = Heavy Chain of 12C6 IgG histidine scanning variant #3
EVQLVQSGGGLVHPGGSLRLSCAGSGFHFSTYLMYWVRQAPGKTLEWVSAIGSGGDTYYADSVKGRFTISRDNAKNSLYLQMNSLRAEDMAVYYCARGLGYWGQGTLVTVSS

SEQ ID NO: 240 - protein, 112 aa, synthetic construct
  REGION 1..112: note = Heavy Chain of 12C6 IgG histidine scanning variant #4
EVQLVQSGGGLVHPGGSLRLSCAGSGFTHSTYLMYWVRQAPGKTLEWVSAIGSGGDTYYADSVKGRFTISRDNAKNSLYLQMNSLRAEDMAVYYCARGLGYWGQGTLVTVSS

SEQ ID NO: 241 - protein, 112 aa, synthetic construct
  REGION 1..112: note = Heavy Chain of 12C6 IgG histidine scanning variant #5
EVQLVQSGGGLVHPGGSLRLSCAGSGFTFHTYLMYWVRQAPGKTLEWVSAIGSGGDTYYADSVKGRFTISRDNAKNSLYLQMNSLRAEDMAVYYCARGLGYWGQGTLVTVSS

SEQ ID NO: 242 - protein, 112 aa, synthetic construct
  REGION 1..112: note = Heavy Chain of 12C6 IgG histidine scanning variant #6
EVQLVQSGGGLVHPGGSLRLSCAGSGFTFSHYLMYWVRQAPGKTLEWVSAIGSGGDTYYADSVKGRFTISRDNAKNSLYLQMNSLRAEDMAVYYCARGLGYWGQGTLVTVSS

SEQ ID NO: 243 - protein, 112 aa, synthetic construct
  REGION 1..112: note = Heavy Chain of 12C6 IgG histidine scanning variant #7
EVQLVQSGGGLVHPGGSLRLSCAGSGFTFSTHLMYWVRQAPGKTLEWVSAIGSGGDTYYADSVKGRFTISRDNAKNSLYLQMNSLRAEDMAVYYCARGLGYWGQGTLVTVSS

SEQ ID NO: 244 - protein, 112 aa, synthetic construct
  REGION 1..112: note = Heavy Chain of 12C6 IgG histidine scanning variant #8
EVQLVQSGGGLVHPGGSLRLSCAGSGFTFSTYHMYWVRQAPGKTLEWVSAIGSGGDTYYADSVKGRFTISRDNAKNSLYLQMNSLRAEDMAVYYCARGLGYWGQGTLVTVSS

SEQ ID NO: 245 - protein, 112 aa, synthetic construct
  REGION 1..112: note = Heavy Chain of 12C6 IgG histidine scanning variant #9
EVQLVQSGGGLVHPGGSLRLSCAGSGFTFSTYLHYWVRQAPGKTLEWVSAIGSGGDTYYADSVKGRFTISRDNAKNSLYLQMNSLRAEDMAVYYCARGLGYWGQGTLVTVSS

SEQ ID NO: 246 - protein, 112 aa, synthetic construct
  REGION 1..112: note = Heavy Chain of 12C6 IgG histidine scanning variant #10
EVQLVQSGGGLVHPGGSLRLSCAGSGFTFSTYLMHWVRQAPGKTLEWVSAIGSGGDTYYADSVKGRFTISRDNAKNSLYLQMNSLRAEDMAVYYCARGLGYWGQGTLVTVSS

SEQ ID NO: 247 - protein, 112 aa, synthetic construct
  REGION 1..112: note = Heavy Chain of 12C6 IgG histidine scanning variant #11
EVQLVQSGGGLVHPGGSLRLSCAGSGFTFSTYLMYWVRQAPGKTLEWVSHIGSGGDTYYADSVKGRFTISRDNAKNSLYLQMNSLRAEDMAVYYCARGLGYWGQGTLVTVSS

SEQ ID NO: 248 - protein, 112 aa, synthetic construct
  REGION 1..112: note = Heavy Chain of 12C6 IgG histidine scanning variant #12
EVQLVQSGGGLVHPGGSLRLSCAGSGFTFSTYLMYWVRQAPGKTLEWVSAHGSGGDTYYADSVKGRFTISRDNAKNSLYLQMNSLRAEDMAVYYCARGLGYWGQGTLVTVSS

SEQ ID NO: 249 - protein, 112 aa, synthetic construct
  REGION 1..112: note = Heavy Chain of 12C6 IgG histidine scanning variant #13
EVQLVQSGGGLVHPGGSLRLSCAGSGFTFSTYLMYWVRQAPGKTLEWVSAIHSGGDTYYADSVKGRFTISRDNAKNSLYLQMNSLRAEDMAVYYCARGLGYWGQGTLVTVSS

SEQ ID NO: 250 - protein, 112 aa, synthetic construct
  REGION 1..112: note = Heavy Chain of 12C6 IgG histidine scanning variant #14
EVQLVQSGGGLVHPGGSLRLSCAGSGFTFSTYLMYWVRQAPGKTLEWVSAIGHGGDTYYADSVKGRFTISRDNAKNSLYLQMNSLRAEDMAVYYCARGLGYWGQGTLVTVSS

SEQ ID NO: 251 - protein, 112 aa, synthetic construct
  REGION 1..112: note = Heavy Chain of 12C6 IgG histidine scanning variant #15
EVQLVQSGGGLVHPGGSLRLSCAGSGFTFSTYLMYWVRQAPGKTLEWVSAIGSHGDTYYADSVKGRFTISRDNAKNSLYLQMNSLRAEDMAVYYCARGLGYWGQGTLVTVSS

SEQ ID NO: 252 - protein, 112 aa, synthetic construct
  REGION 1..112: note = Heavy Chain of 12C6 IgG histidine scanning variant #16
EVQLVQSGGGLVHPGGSLRLSCAGSGFTFSTYLMYWVRQAPGKTLEWVSAIGSGHDTYYADSVKGRFTISRDNAKNSLYLQMNSLRAEDMAVYYCARGLGYWGQGTLVTVSS

SEQ ID NO: 253 - protein, 112 aa, synthetic construct
  REGION 1..112: note = Heavy Chain of 12C6 IgG histidine scanning variant #17
EVQLVQSGGGLVHPGGSLRLSCAGSGFTFSTYLMYWVRQAPGKTLEWVSAIGSGGHTYYADSVKGRFTISRDNAKNSLYLQMNSLRAEDMAVYYCARGLGYWGQGTLVTVSS

SEQ ID NO: 254 - protein, 112 aa, synthetic construct
  REGION 1..112: note = Heavy Chain of 12C6 IgG histidine scanning variant #18
EVQLVQSGGGLVHPGGSLRLSCAGSGFTFSTYLMYWVRQAPGKTLEWVSAIGSGGDHYYADSVKGRFTISRDNAKNSLYLQMNSLRAEDMAVYYCARGLGYWGQGTLVTVSS

SEQ ID NO: 255 - protein, 112 aa, synthetic construct
  REGION 1..112: note = Heavy Chain of 12C6 IgG histidine scanning variant #19
EVQLVQSGGGLVHPGGSLRLSCAGSGFTFSTYLMYWVRQAPGKTLEWVSAIGSGGDTHYADSVKGRFTISRDNAKNSLYLQMNSLRAEDMAVYYCARGLGYWGQGTLVTVSS

SEQ ID NO: 256 - protein, 112 aa, synthetic construct
  REGION 1..112: note = Heavy Chain of 12C6 IgG histidine scanning variant #20
EVQLVQSGGGLVHPGGSLRLSCAGSGFTFSTYLMYWVRQAPGKTLEWVSAIGSGGDTYHADSVKGRFTISRDNAKNSLYLQMNSLRAEDMAVYYCARGLGYWGQGTLVTVSS

SEQ ID NO: 257 - protein, 112 aa, synthetic construct
  REGION 1..112: note = Heavy Chain of 12C6 IgG histidine scanning variant #21
EVQLVQSGGGLVHPGGSLRLSCAGSGFTFSTYLMYWVRQAPGKTLEWVSAIGSGGDTYYHDSVKGRFTISRDNAKNSLYLQMNSLRAEDMAVYYCARGLGYWGQGTLVTVSS

SEQ ID NO: 258 - protein, 112 aa, synthetic construct
  REGION 1..112: note = Heavy Chain of 12C6 IgG histidine scanning variant #22
EVQLVQSGGGLVHPGGSLRLSCAGSGFTFSTYLMYWVRQAPGKTLEWVSAIGSGGDTYYAHSVKGRFTISRDNAKNSLYLQMNSLRAEDMAVYYCARGLGYWGQGTLVTVSS

SEQ ID NO: 259 - protein, 112 aa, synthetic construct
  REGION 1..112: note = Heavy Chain of 12C6 IgG histidine scanning variant #23
EVQLVQSGGGLVHPGGSLRLSCAGSGFTFSTYLMYWVRQAPGKTLEWVSAIGSGGDTYYADHVKGRFTISRDNAKNSLYLQMNSLRAEDMAVYYCARGLGYWGQGTLVTVSS

SEQ ID NO: 260 - protein, 112 aa, synthetic construct
  REGION 1..112: note = Heavy Chain of 12C6 IgG histidine scanning variant #24
EVQLVQSGGGLVHPGGSLRLSCAGSGFTFSTYLMYWVRQAPGKTLEWVSAIGSGGDTYYADSHKGRFTISRDNAKNSLYLQMNSLRAEDMAVYYCARGLGYWGQGTLVTVSS

SEQ ID NO: 261 - protein, 112 aa, synthetic construct
  REGION 1..112: note = Heavy Chain of 12C6 IgG histidine scanning variant #25
EVQLVQSGGGLVHPGGSLRLSCAGSGFTFSTYLMYWVRQAPGKTLEWVSAIGSGGDTYYADSVHGRFTISRDNAKNSLYLQMNSLRAEDMAVYYCARGLGYWGQGTLVTVSS

SEQ ID NO: 262 - protein, 112 aa, synthetic construct
  REGION 1..112: note = Heavy Chain of 12C6 IgG histidine scanning variant #26
EVQLVQSGGGLVHPGGSLRLSCAGSGFTFSTYLMYWVRQAPGKTLEWVSAIGSGGDTYYADSVKHRFTISRDNAKNSLYLQMNSLRAEDMAVYYCARGLGYWGQGTLVTVSS

SEQ ID NO: 263 - protein, 112 aa, synthetic construct
  REGION 1..112: note = Heavy Chain of 12C6 IgG histidine scanning variant #27
EVQLVQSGGGLVHPGGSLRLSCAGSGFTFSTYLMYWVRQAPGKTLEWVSAIGSGGDTYYADSVKGRFTISRDNAKNSLYLQMNSLRAEDMAVYYCHRGLGYWGQGTLVTVSS

SEQ ID NO: 264 - protein, 112 aa, synthetic construct
  REGION 1..112: note = Heavy Chain of 12C6 IgG histidine scanning variant #28
EVQLVQSGGGLVHPGGSLRLSCAGSGFTFSTYLMYWVRQAPGKTLEWVSAIGSGGDTYYADSVKGRFTISRDNAKNSLYLQMNSLRAEDMAVYYCAHGLGYWGQGTLVTVSS

SEQ ID NO: 265 - protein, 112 aa, synthetic construct
  REGION 1..112: note = Heavy Chain of 12C6 IgG histidine scanning variant #29
EVQLVQSGGGLVHPGGSLRLSCAGSGFTFSTYLMYWVRQAPGKTLEWVSAIGSGGDTYYADSVKGRFTISRDNAKNSLYLQMNSLRAEDMAVYYCARHLGYWGQGTLVTVSS

SEQ ID NO: 266 - protein, 112 aa, synthetic construct
  REGION 1..112: note = Heavy Chain of 12C6 IgG histidine scanning variant #30
EVQLVQSGGGLVHPGGSLRLSCAGSGFTFSTYLMYWVRQAPGKTLEWVSAIGSGGDTYYADSVKGRFTISRDNAKNSLYLQMNSLRAEDMAVYYCARGHGYWGQGTLVTVSS

SEQ ID NO: 267 - protein, 112 aa, synthetic construct
  REGION 1..112: note = Heavy Chain of 12C6 IgG histidine scanning variant #31
EVQLVQSGGGLVHPGGSLRLSCAGSGFTFSTYLMYWVRQAPGKTLEWVSAIGSGGDTYYADSVKGRFTISRDNAKNSLYLQMNSLRAEDMAVYYCARGLHYWGQGTLVTVSS

SEQ ID NO: 268 - protein, 112 aa, synthetic construct
  REGION 1..112: note = Heavy Chain of 12C6 IgG histidine scanning variant #32
EVQLVQSGGGLVHPGGSLRLSCAGSGFTFSTYLMYWVRQAPGKTLEWVSAIGSGGDTYYADSVKGRFTISRDNAKNSLYLQMNSLRAEDMAVYYCARGLGHWGQGTLVTVSS

SEQ ID NO: 269 - protein, 109 aa, synthetic construct
  REGION 1..109: note = Light Chain of 12C6 IgG histidine scanning variant #1
EIVLTQSPGTLSLSPGERATLSCHASQSVSSSYLAWYQQKPGQAPRLLIYGASSRATGIPDRFSGSGSGTDFTLTISRLEPEDFAVYYCQQYGSSPMYTFGQGTKLEIK

SEQ ID NO: 270 - protein, 109 aa, synthetic construct
  REGION 1..109: note = Light Chain of 12C6 IgG histidine scanning variant #2
EIVLTQSPGTLSLSPGERATLSCRHSQSVSSSYLAWYQQKPGQAPRLLIYGASSRATGIPDRFSGSGSGTDFTLTISRLEPEDFAVYYCQQYGSSPMYTFGQGTKLEIK

SEQ ID NO: 271 - protein, 109 aa, synthetic construct
  REGION 1..109: note = Light Chain of 12C6 IgG histidine scanning variant #3
EIVLTQSPGTLSLSPGERATLSCRAHQSVSSSYLAWYQQKPGQAPRLLIYGASSRATGIPDRFSGSGSGTDFTLTISRLEPEDFAVYYCQQYGSSPMYTFGQGTKLEIK

SEQ ID NO: 272 - protein, 109 aa, synthetic construct
  REGION 1..109: note = Light Chain of 12C6 IgG histidine scanning variant #4
EIVLTQSPGTLSLSPGERATLSCRASHSVSSSYLAWYQQKPGQAPRLLIYGASSRATGIPDRFSGSGSGTDFTLTISRLEPEDFAVYYCQQYGSSPMYTFGQGTKLEIK

SEQ ID NO: 273 - protein, 109 aa, synthetic construct
  REGION 1..109: note = Light Chain of 12C6 IgG histidine scanning variant #5
EIVLTQSPGTLSLSPGERATLSCRASQHVSSSYLAWYQQKPGQAPRLLIYGASSRATGIPDRFSGSGSGTDFTLTISRLEPEDFAVYYCQQYGSSPMYTFGQGTKLEIK

SEQ ID NO: 274 - protein, 109 aa, synthetic construct
  REGION 1..109: note = Light Chain of 12C6 IgG histidine scanning variant #6
EIVLTQSPGTLSLSPGERATLSCRASQSHSSSYLAWYQQKPGQAPRLLIYGASSRATGIPDRFSGSGSGTDFTLTISRLEPEDFAVYYCQQYGSSPMYTFGQGTKLEIK

SEQ ID NO: 275 - protein, 109 aa, synthetic construct
  REGION 1..109: note = Light Chain of 12C6 IgG histidine scanning variant #7
EIVLTQSPGTLSLSPGERATLSCRASQSVHSSYLAWYQQKPGQAPRLLIYGASSRATGIPDRFSGSGSGTDFTLTISRLEPEDFAVYYCQQYGSSPMYTFGQGTKLEIK

SEQ ID NO: 276 - protein, 109 aa, synthetic construct
  REGION 1..109: note = Light Chain of 12C6 IgG histidine scanning variant #8
EIVLTQSPGTLSLSPGERATLSCRASQSVSHSYLAWYQQKPGQAPRLLIYGASSRATGIPDRFSGSGSGTDFTLTISRLEPEDFAVYYCQQYGSSPMYTFGQGTKLEIK

SEQ ID NO: 277 - protein, 109 aa, synthetic construct
  REGION 1..109: note = Light Chain of 12C6 IgG histidine scanning variant #9
EIVLTQSPGTLSLSPGERATLSCRASQSVSSHYLAWYQQKPGQAPRLLIYGASSRATGIPDRFSGSGSGTDFTLTISRLEPEDFAVYYCQQYGSSPMYTFGQGTKLEIK

SEQ ID NO: 278 - protein, 109 aa, synthetic construct
  REGION 1..109: note = Light Chain of 12C6 IgG histidine scanning variant #10
EIVLTQSPGTLSLSPGERATLSCRASQSVSSSHLAWYQQKPGQAPRLLIYGASSRATGIPDRFSGSGSGTDFTLTISRLEPEDFAVYYCQQYGSSPMYTFGQGTKLEIK

SEQ ID NO: 279 - protein, 109 aa, synthetic construct
  REGION 1..109: note = Light Chain of 12C6 IgG histidine scanning variant #11
EIVLTQSPGTLSLSPGERATLSCRASQSVSSSYHAWYQQKPGQAPRLLIYGASSRATGIPDRFSGSGSGTDFTLTISRLEPEDFAVYYCQQYGSSPMYTFGQGTKLEIK

SEQ ID NO: 280 - protein, 109 aa, synthetic construct
  REGION 1..109: note = Light Chain of 12C6 IgG histidine scanning variant #12
EIVLTQSPGTLSLSPGERATLSCRASQSVSSSYLHWYQQKPGQAPRLLIYGASSRATGIPDRFSGSGSGTDFTLTISRLEPEDFAVYYCQQYGSSPMYTFGQGTKLEIK

SEQ ID NO: 281 - protein, 109 aa, synthetic construct
  REGION 1..109: note = Light Chain of 12C6 IgG histidine scanning variant #13
EIVLTQSPGTLSLSPGERATLSCRASQSVSSSYLAWYQQKPGQAPRLLIYHASSRATGIPDRFSGSGSGTDFTLTISRLEPEDFAVYYCQQYGSSPMYTFGQGTKLEIK

SEQ ID NO: 282 - protein, 109 aa, synthetic construct
  REGION 1..109: note = Light Chain of 12C6 IgG histidine scanning variant #14
EIVLTQSPGTLSLSPGERATLSCRASQSVSSSYLAWYQQKPGQAPRLLIYGHSSRATGIPDRFSGSGSGTDFTLTISRLEPEDFAVYYCQQYGSSPMYTFGQGTKLEIK

SEQ ID NO: 283 - protein, 109 aa, synthetic construct
  REGION 1..109: note = Light Chain of 12C6 IgG histidine scanning variant #15
EIVLTQSPGTLSLSPGERATLSCRASQSVSSSYLAWYQQKPGQAPRLLIYGAHSRATGIPDRFSGSGSGTDFTLTISRLEPEDFAVYYCQQYGSSPMYTFGQGTKLEIK

SEQ ID NO: 284 - protein, 109 aa, synthetic construct
  REGION 1..109: note = Light Chain of 12C6 IgG histidine scanning variant #16
EIVLTQSPGTLSLSPGERATLSCRASQSVSSSYLAWYQQKPGQAPRLLIYGASHRATGIPDRFSGSGSGTDFTLTISRLEPEDFAVYYCQQYGSSPMYTFGQGTKLEIK

SEQ ID NO: 285 - protein, 109 aa, synthetic construct
  REGION 1..109: note = Light Chain of 12C6 IgG histidine scanning variant #17
EIVLTQSPGTLSLSPGERATLSCRASQSVSSSYLAWYQQKPGQAPRLLIYGASSHATGIPDRFSGSGSGTDFTLTISRLEPEDFAVYYCQQYGSSPMYTFGQGTKLEIK

SEQ ID NO: 286 - protein, 109 aa, synthetic construct
  REGION 1..109: note = Light Chain of 12C6 IgG histidine scanning variant #18
EIVLTQSPGTLSLSPGERATLSCRASQSVSSSYLAWYQQKPGQAPRLLIYGASSRHTGIPDRFSGSGSGTDFTLTISRLEPEDFAVYYCQQYGSSPMYTFGQGTKLEIK

SEQ ID NO: 287 - protein, 109 aa, synthetic construct
  REGION 1..109: note = Light Chain of 12C6 IgG histidine scanning variant #19
EIVLTQSPGTLSLSPGERATLSCRASQSVSSSYLAWYQQKPGQAPRLLIYGASSRAHGIPDRFSGSGSGTDFTLTISRLEPEDFAVYYCQQYGSSPMYTFGQGTKLEIK

SEQ ID NO: 288 - protein, 109 aa, synthetic construct
  REGION 1..109: note = Light Chain of 12C6 IgG histidine scanning variant #20
EIVLTQSPGTLSLSPGERATLSCRASQSVSSSYLAWYQQKPGQAPRLLIYGASSRATGIPDRFSGSGSGTDFTLTISRLEPEDFAVYYCHQYGSSPMYTFGQGTKLEIK

SEQ ID NO: 289 - protein, 109 aa, synthetic construct
  REGION 1..109: note = Light Chain of 12C6 IgG histidine scanning variant #21
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSPMYTFGQGTKLEIK

SEQ ID NO: 290 - protein, 109 aa, synthetic construct
  REGION 1..109: note = Light Chain of 12C6 IgG histidine scanning variant #22
EIVLTQSPGTLSLSPGERATLSCRASQSVSSSYLAWYQQKPGQAPRLLIYGASSRATGIPDRFSGSGSGTDFTLTISRLEPEDFAVYYCQQHGSSPMYTFGQGTKLEIK

SEQ ID NO: 291 - protein, 109 aa, synthetic construct
  REGION 1..109: note = Light Chain of 12C6 IgG histidine scanning variant #23
EIVLTQSPGTLSLSPGERATLSCRASQSVSSSYLAWYQQKPGQAPRLLIYGASSRATGIPDRFSGSGSGTDFTLTISRLEPEDFAVYYCQQYHSSPMYTFGQGTKLEIK

SEQ ID NO: 292 - protein, 109 aa, synthetic construct
  REGION 1..109: note = Light Chain of 12C6 IgG histidine scanning variant #24
EIVLTQSPGTLSLSPGERATLSCRASQSVSSSYLAWYQQKPGQAPRLLIYGASSRATGIPDRFSGSGSGTDFTLTISRLEPEDFAVYYCQQYGHSPMYTFGQGTKLEIK

SEQ ID NO: 293 - protein, 109 aa, synthetic construct
  REGION 1..109: note = Light Chain of 12C6 IgG histidine scanning variant #25
EIVLTQSPGTLSLSPGERATLSCRASQSVSSSYLAWYQQKPGQAPRLLIYGASSRATGIPDRFSGSGSGTDFTLTISRLEPEDFAVYYCQQYGSHPMYTFGQGTKLEIK

SEQ ID NO: 294 - protein, 109 aa, synthetic construct
  REGION 1..109: note = Light Chain of 12C6 IgG histidine scanning variant #26
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSHMYTFGQGTKLEIK

SEQ ID NO: 295 - protein, 109 aa, synthetic construct
  REGION 1..109: note = Light Chain of 12C6 IgG histidine scanning variant #27
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPHYTFGQGTKLEIK

SEQ ID NO: 296 - protein, 109 aa, synthetic construct
  REGION 1..109: note = Light Chain of 12C6 IgG histidine scanning variant #28
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPMHTFGQGTKLEIK

SEQ ID NO: 297 - protein, 109 aa, synthetic construct
  REGION 1..109: note = Light Chain of 12C6 IgG histidine scanning variant #29
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPMYHFGQGTKLEIK

SEQ ID NO: 298 - protein, 1070 aa, synthetic construct; identical to SEQ ID NO: 9
  REGION 1..1070: note = Human PTK7

SEQ ID NO: 299 - protein, 1040 aa, synthetic construct
  REGION 1..1040: note = Mature Human PTK7
AIVFIKQPSSQDALQGRRALLRCEVEAPGPVHVYWLLDGAPVQDTERRFAQGSSLSFAAVDRLQDSGTFQCVARDDVTGEEARSANASFNIKWIEAGPVVLKHPASEAEIQPQTQVTLRCHIDGHPRPTYQWFRDGTPLSDGQSNHTVSSKERNLTLRPAGPEHSGLYSCCAHSAFGQACSSQNFTLSIADESFARVVLAPQDVVVARYEEAMFHCQFSAQPPPSLQWLFEDETPITNRSRPPHLRRATVFANGSLLLTQVRPRNAGIYRCIGQGQRGPPIILEATLHLAEIEDMPLFEPRVFTAGSEERVTCLPPKGLPEPSVWWEHAGVRLPTHGRVYQKGHELVLANIAESDAGVYTCHAANLAGQRRQDVNITVATVPSWLKKPQDSQLEEGKPGYLDCLTQATPKPTVVWYRNQMLISEDSRFEVFKNGTLRINSVEVYDGTWYRCMSSTPAGSIEAQARVQVLEKLKFTPPPQPQQCMEFDKEATVPCSATGREKPTIKWERADGSSLPEWVTDNAGTLHFARVTRDDAGNYTCIASNGPQGQIRAHVQLTVAVFITFKVEPERTTVYQGHTALLQCEAQGDPKPLIQWKGKDRILDPTKLGPRMHIFQNGSLVIHDVAPEDSGRYTCIAGNSCNIKHTEAPLYVVDKPVPEESEGPGSPPPYKMIQTIGLSVGAAVAYIIAVLGLMFYCKKRCKAKRLQKQPEGEEPEMECLNGGPLQNGQPSAEIQEEVALTSLGSGPAATNKRHSTSDKMHFPRSSLQPITTLGKSEFGEVFLAKAQGLEEGVAETLVLVKSLQSKDEQQQLDFRRELEMFGKLNHANVVRLLGLCREAEPHYMVLEYVDLGDLKQFLRISKSKDEKLKSQPLSTKQKVALCTQVALGMEHLSNNRFVHKDLAARNCLVSAQRQVKVSALGLSKDVYNSEYYHFRQAWVPLRWMSPEAILEGDFSTKSDVWAFGVLMWEVFTHGEMPHGGQADDEVLADLQAGKARLPQPEGCPSKLYRLMQRCWALSPKDRPSFSEIASALGDSTVDSKP

SEQ ID NO: 300 - protein, 674 aa, synthetic construct; identical to SEQ ID NO: 11
  REGION 1..674: note = Extracellular domain of PTK7

SEQ ID NO: 301 - protein, 112 aa, synthetic construct
  REGION 1..112: note = VH domain of humanized rabbit IgG_9345
EVQLVESGGGLVQPGGSLRLSCTASGFSLSNYVMNWVRQAPGKGLEWVGIIDSSGSTYYASWAKGRFTISRDTSKNTVYLQMNSLRAEDTAVYFCARGLYLWGQGTLVTVSS

SEQ ID NO: 302 - protein, 110 aa, synthetic construct
  REGION 1..110: note = VL domain of humanized rabbit IgG_9345
EIVMTQSPSTLSASVGDRVIITCQSSQSVYNNNLLAWYQQKPGKAPKLLIYYTSNLASGVPSRFSGSGSGAEFTLTISSLQPDDFATYYCQGGYNGNIYAFGQGTKLTVL

SEQ ID NO: 303 - protein, 10 aa, synthetic construct
  REGION 1..10: note = Heavy Chain CDR1 of rabbit 9345 IgG
GFSLSNYVMN

SEQ ID NO: 304 - protein, 16 aa, synthetic construct
  REGION 1..16: note = Heavy Chain CDR2 of rabbit 9345 IgG
IIDSSGSTYYASWAKG

SEQ ID NO: 305 - protein, 6 aa, synthetic construct
  REGION 1..6: note = Heavy Chain CDR3 of rabbit 9345 IgG
ARGLYL

SEQ ID NO: 306 - protein, 13 aa, synthetic construct
  REGION 1..13: note = Light Chain CDR1 of rabbit 9345 IgG
QSSQSVYNNNLLA

SEQ ID NO: 307 - protein, 7 aa, synthetic construct
  REGION 1..7: note = Light Chain CDR2 of rabbit 9345 IgG
YTSNLAS

SEQ ID NO: 308 - protein, 10 aa, synthetic construct
  REGION 1..10: note = Light Chain CDR3 of rabbit 9345 IgG
QGGYNGNIYA

SEQ ID NO: 309 - protein, 112 aa, synthetic construct
  REGION 1..112: note = VH domain of 9345 IgG histidine scanning variant #1
EVQLVESGGGLVQPGGSLRLSCTASHFSLSNYVMNWVRQAPGKGLEWVGIIDSSGSTYYASWAKGRFTISRDTSKNTVYLQMNSLRAEDTAVYFCARGLYLWGQGTLVTVSS

SEQ ID NO: 310 - protein, 112 aa, synthetic construct
  REGION 1..112: note = VH domain of 9345 IgG histidine scanning variant #2
EVQLVESGGGLVQPGGSLRLSCTASGHSLSNYVMNWVRQAPGKGLEWVGIIDSSGSTYYASWAKGRFTISRDTSKNTVYLQMNSLRAEDTAVYFCARGLYLWGQGTLVTVSS

SEQ ID NO: 311 - protein, 112 aa, synthetic construct
  REGION 1..112: note = VH domain of 9345 IgG histidine scanning variant #3
EVQLVESGGGLVQPGGSLRLSCTASGFHLSNYVMNWVRQAPGKGLEWVGIIDSSGSTYYASWAKGRFTISRDTSKNTVYLQMNSLRAEDTAVYFCARGLYLWGQGTLVTVSS

SEQ ID NO: 312 - protein, 112 aa, synthetic construct
  REGION 1..112: note = VH domain of 9345 IgG histidine scanning variant #4
EVQLVESGGGLVQPGGSLRLSCTASGFSHSNYVMNWVRQAPGKGLEWVGIIDSSGSTYYASWAKGRFTISRDTSKNTVYLQMNSLRAEDTAVYFCARGLYLWGQGTLVTVSS

SEQ ID NO: 313 - protein, 112 aa, synthetic construct
  REGION 1..112: note = VH domain of 9345 IgG histidine scanning variant #5
EVQLVESGGGLVQPGGSLRLSCTASGFSLHNYVMNWVRQAPGKGLEWVGIIDSSGSTYYASWAKGRFTISRDTSKNTVYLQMNSLRAEDTAVYFCARGLYLWGQGTLVTVSS

SEQ ID NO: 314 - protein, 112 aa, synthetic construct
  REGION 1..112: note = VH domain of 9345 IgG histidine scanning variant #6
EVQLVESGGGLVQPGGSLRLSCTASGFSLSHYVMNWVRQAPGKGLEWVGIIDSSGSTYYASWAKGRFTISRDTSKNTVYLQMNSLRAEDTAVYFCARGLYLWGQGTLVTVSS

SEQ ID NO: 315 - protein, 112 aa, synthetic construct
  REGION 1..112: note = VH domain of 9345 IgG histidine scanning variant #7
EVQLVESGGGLVQPGGSLRLSCTASGFSLSNHVMNWVRQAPGKGLEWVGIIDSSGSTYYASWAKGRFTISRDTSKNTVYLQMNSLRAEDTAVYFCARGLYLWGQGTLVTVSS

SEQ ID NO: 316 - protein, 112 aa, synthetic construct
  REGION 1..112: note = VH domain of 9345 IgG histidine scanning variant #8
EVQLVESGGGLVQPGGSLRLSCTASGFSLSNYHMNWVRQAPGKGLEWVGIIDSSGSTYYASWAKGRFTISRDTSKNTVYLQMNSLRAEDTAVYFCARGLYLWGQGTLVTVSS

SEQ ID NO: 317 - protein, 112 aa, synthetic construct
  REGION 1..112: note = VH domain of 9345 IgG histidine scanning variant #9
EVQLVESGGGLVQPGGSLRLSCTASGFSLSNYVHNWVRQAPGKGLEWVGIIDSSGSTYYASWAKGRFTISRDTSKNTVYLQMNSLRAEDTAVYFCARGLYLWGQGTLVTVSS

SEQ ID NO: 318 - protein, 112 aa, synthetic construct
  REGION 1..112: note = VH domain of 9345 IgG histidine scanning variant #10
EVQLVESGGGLVQPGGSLRLSCTASGFSLSNYVMHWVRQAPGKGLEWVGIIDSSGSTYYASWAKGRFTISRDTSKNTVYLQMNSLRAEDTAVYFCARGLYLWGQGTLVTVSS

SEQ ID NO: 319 - protein, 112 aa, synthetic construct
  REGION 1..112: note = VH domain of 9345 IgG histidine scanning variant #11
EVQLVESGGGLVQPGGSLRLSCTASGFSLSNYVMNWVRQAPGKGLEWVGHIDSSGSTYYASWAKGRFTISRDTSKNTVYLQMNSLRAEDTAVYFCARGLYLWGQGTLVTVSS

SEQ ID NO: 320 - protein, 112 aa, synthetic construct
  REGION 1..112: note = VH domain of 9345 IgG histidine scanning variant #12
EVQLVESGGGLVQPGGSLRLSCTASGFSLSNYVMNWVRQAPGKGLEWVGIHDSSGSTYYASWAKGRFTISRDTSKNTVYLQMNSLRAEDTAVYFCARGLYLWGQGTLVTVSS

SEQ ID NO: 321 - protein, 112 aa, synthetic construct
  REGION 1..112: note = VH domain of 9345 IgG histidine scanning variant #13
EVQLVESGGGLVQPGGSLRLSCTASGFSLSNYVMNWVRQAPGKGLEWVGIIHSSGSTYYASWAKGRFTISRDTSKNTVYLQMNSLRAEDTAVYFCARGLYLWGQGTLVTVSS

SEQ ID NO: 322 - protein, 112 aa, synthetic construct
  REGION 1..112: note = VH domain of 9345 IgG histidine scanning variant #14
EVQLVESGGGLVQPGGSLRLSCTASGFSLSNYVMNWVRQAPGKGLEWVGIIDHSGSTYYASWAKGRFTISRDTSKNTVYLQMNSLRAEDTAVYFCARGLYLWGQGTLVTVSS

SEQ ID NO: 323 - protein, 112 aa, synthetic construct
  REGION 1..112: note = VH domain of 9345 IgG histidine scanning variant #15
EVQLVESGGGLVQPGGSLRLSCTASGFSLSNYVMNWVRQAPGKGLEWVGIIDSHGSTYYASWAKGRFTISRDTSKNTVYLQMNSLRAEDTAVYFCARGLYLWGQGTLVTVSS

SEQ ID NO: 324 - protein, 112 aa, synthetic construct
  REGION 1..112: note = VH domain of 9345 IgG histidine scanning variant #16
EVQLVESGGGLVQPGGSLRLSCTASGFSLSNYVMNWVRQAPGKGLEWVGIIDSSHSTYYASWAKGRFTISRDTSKNTVYLQMNSLRAEDTAVYFCARGLYLWGQGTLVTVSS

SEQ ID NO: 325 - protein, 112 aa, synthetic construct
  REGION 1..112: note = VH domain of 9345 IgG histidine scanning variant #17
EVQLVESGGGLVQPGGSLRLSCTASGFSLSNYVMNWVRQAPGKGLEWVGIIDSSGHTYYASWAKGRFTISRDTSKNTVYLQMNSLRAEDTAVYFCARGLYLWGQGTLVTVSS

SEQ ID NO: 326 - protein, 112 aa, synthetic construct
  REGION 1..112: note = VH domain of 9345 IgG histidine scanning variant #18
EVQLVESGGGLVQPGGSLRLSCTASGFSLSNYVMNWVRQAPGKGLEWVGIIDSSGSHYYASWAKGRFTISRDTSKNTVYLQMNSLRAEDTAVYFCARGLYLWGQGTLVTVSS

SEQ ID NO: 327 - protein, 112 aa, synthetic construct
  REGION 1..112: note = VH domain of 9345 IgG histidine scanning variant #19
EVQLVESGGGLVQPGGSLRLSCTASGFSLSNYVMNWVRQAPGKGLEWVGIIDSSGSTHYASWAKGRFTISRDTSKNTVYLQMNSLRAEDTAVYFCARGLYLWGQGTLVTVSS

SEQ ID NO: 328 - protein, 112 aa, synthetic construct
  REGION 1..112: note = VH domain of 9345 IgG histidine scanning variant #20
EVQLVESGGGLVQPGGSLRLSCTASGFSLSNYVMNWVRQAPGKGLEWVGIIDSSGSTYHASWAKGRFTISRDTSKNTVYLQMNSLRAEDTAVYFCARGLYLWGQGTLVTVSS

SEQ ID NO: 329 - protein, 112 aa, synthetic construct
  REGION 1..112: note = VH domain of 9345 IgG histidine scanning variant #21
EVQLVESGGGLVQPGGSLRLSCTASGFSLSNYVMNWVRQAPGKGLEWVGIIDSSGSTYYHSWAKGRFTISRDTSKNTVYLQMNSLRAEDTAVYFCARGLYLWGQGTLVTVSS

SEQ ID NO: 330 - protein, 112 aa, synthetic construct
  REGION 1..112: note = VH domain of 9345 IgG histidine scanning variant #22
EVQLVESGGGLVQPGGSLRLSCTASGFSLSNYVMNWVRQAPGKGLEWVGIIDSSGSTYYAHWAKGRFTISRDTSKNTVYLQMNSLRAEDTAVYFCARGLYLWGQGTLVTVSS

SEQ ID NO: 331 - protein, 112 aa, synthetic construct
  REGION 1..112: note = VH domain of 9345 IgG histidine scanning variant #23
EVQLVESGGGLVQPGGSLRLSCTASGFSLSNYVMNWVRQAPGKGLEWVGIIDSSGSTYYASHAKGRFTISRDTSKNTVYLQMNSLRAEDTAVYFCARGLYLWGQGTLVTVSS

SEQ ID NO: 332 - protein, 112 aa, synthetic construct
  REGION 1..112: note = VH domain of 9345 IgG histidine scanning variant #24
EVQLVESGGGLVQPGGSLRLSCTASGFSLSNYVMNWVRQAPGKGLEWVGIIDSSGSTYYASWHKGRFTISRDTSKNTVYLQMNSLRAEDTAVYFCARGLYLWGQGTLVTVSS

SEQ ID NO: 333 - protein, 112 aa, synthetic construct
  REGION 1..112: note = VH domain of 9345 IgG histidine scanning variant #25
EVQLVESGGGLVQPGGSLRLSCTASGFSLSNYVMNWVRQAPGKGLEWVGIIDSSGSTYYASWAHGRFTISRDTSKNTVYLQMNSLRAEDTAVYFCARGLYLWGQGTLVTVSS

SEQ ID NO: 334 - protein, 112 aa, synthetic construct
  REGION 1..112: note = VH domain of 9345 IgG histidine scanning variant #26
EVQLVESGGGLVQPGGSLRLSCTASGFSLSNYVMNWVRQAPGKGLEWVGIIDSSGSTYYASWAKHRFTISRDTSKNTVYLQMNSLRAEDTAVYFCARGLYLWGQGTLVTVSS

SEQ ID NO: 335 - protein, 112 aa, synthetic construct
  REGION 1..112: note = VH domain of 9345 IgG histidine scanning variant #27
EVQLVESGGGLVQPGGSLRLSCTASGFSLSNYVMNWVRQAPGKGLEWVGIIDSSGSTYYASWAKGRFTISRDTSKNTVYLQMNSLRAEDTAVYFCHRGLYLWGQGTLVTVSS

SEQ ID NO: 336 - protein, 112 aa, synthetic construct
  REGION 1..112: note = VH domain of 9345 IgG histidine scanning variant #28
EVQLVESGGGLVQPGGSLRLSCTASGFSLSNYVMNWVRQAPGKGLEWVGIIDSSGSTYYASWAKGRFTISRDTSKNTVYLQMNSLRAEDTAVYFCAHGLYLWGQGTLVTVSS

SEQ ID NO: 337 - protein, 112 aa, synthetic construct
  REGION 1..112: note = VH domain of 9345 IgG histidine scanning variant #29
EVQLVESGGGLVQPGGSLRLSCTASGFSLSNYVMNWVRQAPGKGLEWVGIIDSSGSTYYASWAKGRFTISRDTSKNTVYLQMNSLRAEDTAVYFCARHLYLWGQGTLVTVSS

SEQ ID NO: 338 - protein, 112 aa, synthetic construct
  REGION 1..112: note = VH domain of 9345 IgG histidine scanning variant #30
EVQLVESGGGLVQPGGSLRLSCTASGFSLSNYVMNWVRQAPGKGLEWVGIIDSSGSTYYASWAKGRFTISRDTSKNTVYLQMNSLRAEDTAVYFCARGHYLWGQGTLVTVSS

SEQ ID NO: 339 - protein, 112 aa, synthetic construct
  REGION 1..112: note = VH domain of 9345 IgG histidine scanning variant #31
EVQLVESGGGLVQPGGSLRLSCTASGFSLSNYVMNWVRQAPGKGLEWVGIIDSSGSTYYASWAKGRFTISRDTSKNTVYLQMNSLRAEDTAVYFCARGLHLWGQGTLVTVSS

SEQ ID NO: 340 - protein, 112 aa, synthetic construct
  REGION 1..112: note = VH domain of 9345 IgG histidine scanning variant #32
EVQLVESGGGLVQPGGSLRLSCTASGFSLSNYVMNWVRQAPGKGLEWVGIIDSSGSTYYASWAKGRFTISRDTSKNTVYLQMNSLRAEDTAVYFCARGLYHWGQGTLVTVSS

SEQ ID NO: 341 - protein, 112 aa, synthetic construct
  REGION 1..112: note = VH domain of 9345 IgG histidine scanning variant #33
EVQLVESGGGLVQPGGSLRLSCTASGFSLSNYVMNWVRQAPGKGLEWVGIIDSSGSTYYASWAKGRFTISRDTSKNTVYLQMNSLRAEDTAVYYCARGLYLWGQGTLVTVSS

SEQ ID NO: 342 - protein, 112 aa, synthetic construct
  REGION 1..112: note = VH domain of 9345 IgG histidine scanning variant #34
EVQLVESGGGLVQPGGSLRLSCTASGFSLSNYVMNWVRQAPGKGLEWVGIIASSGSTYYASWAKGRFTISRDTSKNTVYLQMNSLRAEDTAVYFCARGLYLWGQGTLVTVSS

SEQ ID NO: 343 - protein, 110 aa, synthetic construct
  REGION 1..110: note = VL domain of 9345 IgG histidine scanning variant #1
EIVMTQSPSTLSASVGDRVIITCHSSQSVYNNNLLAWYQQKPGKAPKLLIYYTSNLASGVPSRFSGSGSGAEFTLTISSLQPDDFATYYCQGGYNGNIYAFGQGTKLTVL

SEQ ID NO: 344 - protein, 110 aa, synthetic construct
  REGION 1..110: note = VL domain of 9345 IgG histidine scanning variant #2
EIVMTQSPSTLSASVGDRVIITCQHSQSVYNNNLLAWYQQKPGKAPKLLIYYTSNLASGVPSRFSGSGSGAEFTLTISSLQPDDFATYYCQGGYNGNIYAFGQGTKLTVL

SEQ ID NO: 345 - protein, 110 aa, synthetic construct
  REGION 1..110: note = VL domain of 9345 IgG histidine scanning variant #3
EIVMTQSPSTLSASVGDRVIITCQSHQSVYNNNLLAWYQQKPGKAPKLLIYYTSNLASGVPSRFSGSGSGAEFTLTISSLQPDDFATYYCQGGYNGNIYAFGQGTKLTVL

SEQ ID NO: 346 - protein, 110 aa, synthetic construct
  REGION 1..110: note = VL domain of 9345 IgG histidine scanning variant #4
EIVMTQSPSTLSASVGDRVIITCQSSHSVYNNNLLAWYQQKPGKAPKLLIYYTSNLASGVPSRFSGSGSGAEFTLTISSLQPDDFATYYCQGGYNGNIYAFGQGTKLTVL

SEQ ID NO: 347 - protein, 110 aa, synthetic construct
  REGION 1..110: note = VL domain of 9345 IgG histidine scanning variant #5
EIVMTQSPSTLSASVGDRVIITCQSSQHVYNNNLLAWYQQKPGKAPKLLIYYTSNLASGVPSRFSGSGSGAEFTLTISSLQPDDFATYYCQGGYNGNIYAFGQGTKLTVL

SEQ ID NO: 348 - protein, 110 aa, synthetic construct
  REGION 1..110: note = VL domain of 9345 IgG histidine scanning variant #6
EIVMTQSPSTLSASVGDRVIITCQSSQSHYNNNLLAWYQQKPGKAPKLLIYYTSNLASGVPSRFSGSGSGAEFTLTISSLQPDDFATYYCQGGYNGNIYAFGQGTKLTVL

SEQ ID NO: 349 - protein, 110 aa, synthetic construct
  REGION 1..110: note = VL domain of 9345 IgG histidine scanning variant #7
EIVMTQSPSTLSASVGDRVIITCQSSQSVHNNNLLAWYQQKPGKAPKLLIYYTSNLASGVPSRFSGSGSGAEFTLTISSLQPDDFATYYCQGGYNGNIYAFGQGTKLTVL

SEQ ID NO: 350 - protein, 110 aa, synthetic construct
  REGION 1..110: note = VL domain of 9345 IgG histidine scanning variant #8
EIVMTQSPSTLSASVGDRVIITCQSSQSVYHNNLLAWYQQKPGKAPKLLIYYTSNLASGVPSRFSGSGSGAEFTLTISSLQPDDFATYYCQGGYNGNIYAFGQGTKLTVL

SEQ ID NO: 351 - protein, 110 aa, synthetic construct
  REGION 1..110: note = VL domain of 9345 IgG histidine scanning variant #9
EIVMTQSPSTLSASVGDRVIITCQSSQSVYNHNLLAWYQQKPGKAPKLLIYYTSNLASGVPSRFSGSGSGAEFTLTISSLQPDDFATYYCQGGYNGNIYAFGQGTKLTVL

SEQ ID NO: 352 - protein, 110 aa, synthetic construct
  REGION 1..110: note = VL domain of 9345 IgG histidine scanning variant #10
EIVMTQSPSTLSASVGDRVIITCQSSQSVYNNHLLAWYQQKPGKAPKLLIYYTSNLASGVPSRFSGSGSGAEFTLTISSLQPDDFATYYCQGGYNGNIYAFGQGTKLTVL

SEQ ID NO: 353 - protein, 110 aa, synthetic construct
  REGION 1..110: note = VL domain of 9345 IgG histidine scanning variant #11
EIVMTQSPSTLSASVGDRVIITCQSSQSVYNNNHLAWYQQKPGKAPKLLIYYTSNLASGVPSRFSGSGSGAEFTLTISSLQPDDFATYYCQGGYNGNIYAFGQGTKLTVL

SEQ ID NO: 354 - protein, 110 aa, synthetic construct
  REGION 1..110: note = VL domain of 9345 IgG histidine scanning variant #12
EIVMTQSPSTLSASVGDRVIITCQSSQSVYNNNLHAWYQQKPGKAPKLLIYYTSNLASGVPSRFSGSGSGAEFTLTISSLQPDDFATYYCQGGYNGNIYAFGQGTKLTVL

SEQ ID NO: 355 - protein, 110 aa, synthetic construct
  REGION 1..110: note = VL domain of 9345 IgG histidine scanning variant #13
EIVMTQSPSTLSASVGDRVIITCQSSQSVYNNNLLHWYQQKPGKAPKLLIYYTSNLASGVPSRFSGSGSGAEFTLTISSLQPDDFATYYCQGGYNGNIYAFGQGTKLTVL

SEQ ID NO: 356 - protein, 110 aa, synthetic construct
  REGION 1..110: note = VL domain of 9345 IgG histidine scanning variant #14
EIVMTQSPSTLSASVGDRVIITCQSSQSVYNNNLLAWYQQKPGKAPKLLIYHTSNLASGVPSRFSGSGSGAEFTLTISSLQPDDFATYYCQGGYNGNIYAFGQGTKLTVL

SEQ ID NO: 357 - protein, 110 aa, synthetic construct
  REGION 1..110: note = VL domain of 9345 IgG histidine scanning variant #15
EIVMTQSPSTLSASVGDRVIITCQSSQSVYNNNLLAWYQQKPGKAPKLLIYYHSNLASGVPSRFSGSGSGAEFTLTISSLQPDDFATYYCQGGYNGNIYAFGQGTKLTVL

SEQ ID NO: 358 - protein, 110 aa, synthetic construct
  REGION 1..110: note = VL domain of 9345 IgG histidine scanning variant #16
EIVMTQSPSTLSASVGDRVIITCQSSQSVYNNNLLAWYQQKPGKAPKLLIYYTHNLASGVPSRFSGSGSGAEFTLTISSLQPDDFATYYCQGGYNGNIYAFGQGTKLTVL

SEQ ID NO: 359 - protein, 110 aa, synthetic construct
  REGION 1..110: note = VL domain of 9345 IgG histidine scanning variant #17
EIVMTQSPSTLSASVGDRVIITCQSSQSVYNNNLLAWYQQKPGKAPKLLIYYTSHLASGVPSRFSGSGSGAEFTLTISSLQPDDFATYYCQGGYNGNIYAFGQGTKLTVL

SEQ ID NO: 360 - protein, 110 aa, synthetic construct
  REGION 1..110: note = VL domain of 9345 IgG histidine scanning variant #18
EIVMTQSPSTLSASVGDRVIITCQSSQSVYNNNLLAWYQQKPGKAPKLLIYYTSNHASGVPSRFSGSGSGAEFTLTISSLQPDDFATYYCQGGYNGNIYAFGQGTKLTVL

SEQ ID NO: 361 - protein, 110 aa, synthetic construct
  REGION 1..110: note = VL domain of 9345 IgG histidine scanning variant #19
EIVMTQSPSTLSASVGDRVIITCQSSQSVYNNNLLAWYQQKPGKAPKLLIYYTSNLHSGVPSRFSGSGSGAEFTLTISSLQPDDFATYYCQGGYNGNIYAFGQGTKLTVL

SEQ ID NO: 362 - protein, 110 aa, synthetic construct
  REGION 1..110: note = VL domain of 9345 IgG histidine scanning variant #20
EIVMTQSPSTLSASVGDRVIITCQSSQSVYNNNLLAWYQQKPGKAPKLLIYYTSNLAHGVPSRFSGSGSGAEFTLTISSLQPDDFATYYCQGGYNGNIYAFGQGTKLTVL

SEQ ID NO: 363 - protein, 110 aa, synthetic construct
  REGION 1..110: note = VL domain of 9345 IgG histidine scanning variant #21
EIVMTQSPSTLSASVGDRVIITCQSSQSVYNNNLLAWYQQKPGKAPKLLIYYTSNLASGVPSRFSGSGSGAEFTLTISSLQPDDFATYYCHGGYNGNIYAFGQGTKLTVL

SEQ ID NO: 364 - protein, 110 aa, synthetic construct
  REGION 1..110: note = VL domain of 9345 IgG histidine scanning variant #22
EIVMTQSPSTLSASVGDRVIITCQSSQSVYNNNLLAWYQQKPGKAPKLLIYYTSNLASGVPSRFSGSGSGAEFTLTISSLQPDDFATYYCQHGYNGNIYAFGQGTKLTVL

SEQ ID NO: 365 - protein, 110 aa, synthetic construct
  REGION 1..110: note = VL domain of 9345 IgG histidine scanning variant #23
EIVMTQSPSTLSASVGDRVIITCQSSQSVYNNNLLAWYQQKPGKAPKLLIYYTSNLASGVPSRFSGSGSGAEFTLTISSLQPDDFATYYCQGHYNGNIYAFGQGTKLTVL

SEQ ID NO: 366 - protein, 110 aa, synthetic construct
  REGION 1..110: note = VL domain of 9345 IgG histidine scanning variant #24
EIVMTQSPSTLSASVGDRVIITCQSSQSVYNNNLLAWYQQKPGKAPKLLIYYTSNLASGVPSRFSGSGSGAEFTLTISSLQPDDFATYYCQGGHNGNIYAFGQGTKLTVL

SEQ ID NO: 367 - protein, 110 aa, synthetic construct
  REGION 1..110: note = VL domain of 9345 IgG histidine scanning variant #25
EIVMTQSPSTLSASVGDRVIITCQSSQSVYNNNLLAWYQQKPGKAPKLLIYYTSNLASGVPSRFSGSGSGAEFTLTISSLQPDDFATYYCQGGYHGNIYAFGQGTKLTVL

SEQ ID NO: 368 - protein, 110 aa, synthetic construct
  REGION 1..110: note = VL domain of 9345 IgG histidine scanning variant #26
EIVMTQSPSTLSASVGDRVIITCQSSQSVYNNNLLAWYQQKPGKAPKLLIYYTSNLASGVPSRFSGSGSGAEFTLTISSLQPDDFATYYCQGGYNHNIYAFGQGTKLTVL

SEQ ID NO: 369 - protein, 110 aa, synthetic construct
  REGION 1..110: note = VL domain of 9345 IgG histidine scanning variant #27
EIVMTQSPSTLSASVGDRVIITCQSSQSVYNNNLLAWYQQKPGKAPKLLIYYTSNLASGVPSRFSGSGSGAEFTLTISSLQPDDFATYYCQGGYNGHIYAFGQGTKLTVL

SEQ ID NO: 370 - protein, 110 aa, synthetic construct
  REGION 1..110: note = VL domain of 9345 IgG histidine scanning variant #28
EIVMTQSPSTLSASVGDRVIITCQSSQSVYNNNLLAWYQQKPGKAPKLLIYYTSNLASGVPSRFSGSGSGAEFTLTISSLQPDDFATYYCQGGYNGNHYAFGQGTKLTVL

SEQ ID NO: 371 - protein, 110 aa, synthetic construct
  REGION 1..110: note = VL domain of 9345 IgG histidine scanning variant #29
EIVMTQSPSTLSASVGDRVIITCQSSQSVYNNNLLAWYQQKPGKAPKLLIYYTSNLASGVPSRFSGSGSGAEFTLTISSLQPDDFATYYCQGGYNGNIHAFGQGTKLTVL

SEQ ID NO: 372 - protein, 110 aa, synthetic construct
  REGION 1..110: note = VL domain of 9345 IgG histidine scanning variant #30
EIVMTQSPSTLSASVGDRVIITCQSSQSVYNNNLLAWYQQKPGKAPKLLIYYTSNLASGVPSRFSGSGSGAEFTLTISSLQPDDFATYYCQGGYNGNIYHFGQGTKLTVL

SEQ ID NO: 373 - protein, 110 aa, synthetic construct
  REGION 1..110: note = VL domain of 9345 IgG histidine scanning variant #31
EIVMTQSPSTLSASVGDRVIITCQSSQSVYNNNLLAWYQQKPGKAPKLLIYYTSNLASGVPSRFSGSGSGAEFTLTISSLQPDDFATYYCQGGYNGNIYAFGQGTKLEIK

SEQ ID NO: 374 - protein, 110 aa, synthetic construct
  REGION 1..110: note = VL domain of 9345 IgG histidine scanning variant #32
EIVMTQSPSTLSASVGDRVIITCQSSQSVYNNNLLAWYQQKPGKAPKLLIYYTSNLASGVPSRFSGSGSGAEFTLTISSLQPDDFATYYCQGGYAGNIYAFGQGTKLTVL

SEQ ID NO: 375 - protein, 119 aa, synthetic construct
  REGION 1..119: note = VH domain of 9359 IgG
EVQLVESGGGLVQPGGSLRLSCTASGFSLSSYAMGWVRQAPGKGLEWVGFINAGGNTYYANWAKGRFTISRDTSKNTVYLQMNSLRAEDTAVYFCARRYSRASGWYDLWGQGTLVTVSS

SEQ ID NO: 376 - protein, 112 aa, synthetic construct
  REGION 1..112: note = VL domain of 9359 IgG
EIVMTQSPSTLSASVGDRVIITCQASQSISSYLAWYQQKPGKAPKLLIYAASTLASGVPSRFSGSGSGAEFTLTISSLQPDDFATYYCQSYSYSSGIDYGDVFGQGTKLTVL

SEQ ID NO: 377 - protein, 10 aa, synthetic construct
  REGION 1..10: note = Heavy Chain CDR1 of 9359 IgG
GFSLSSYAMG

SEQ ID NO: 378 - protein, 16 aa, synthetic construct
  REGION 1..16: note = Heavy Chain CDR2 of 9359 IgG
FINAGGNTYYANWAKG

SEQ ID NO: 379 - protein, 13 aa, synthetic construct
  REGION 1..13: note = Heavy Chain CDR3 of 9359 IgG
ARRYSRASGWYDL

SEQ ID NO: 380 - protein, 11 aa, synthetic construct
  REGION 1..11: note = Light Chain CDR1 of 9359 IgG
QASQSISSYLA

SEQ ID NO: 381 - protein, 7 aa, synthetic construct
  REGION 1..7: note = Light Chain CDR2 of 9359 IgG
AASTLAS

SEQ ID NO: 382 - protein, 14 aa, synthetic construct
  REGION 1..14: note = Light Chain CDR3 of 9359 IgG
QSYSYSSGIDYGDV

SEQ ID NO: 383 - protein, 119 aa, synthetic construct
  REGION 1..119: note = VH domain of 9359 IgG histidine scanning variant #1
EVQLVESGGGLVQPGGSLRLSCTASHFSLSSYAMGWVRQAPGKGLEWVGFINAGGNTYYANWAKGRFTISRDTSKNTVYLQMNSLRAEDTAVYFCARRYSRASGWYDLWGQGTLVTVSS

SEQ ID NO: 384 - protein, 119 aa, synthetic construct
  REGION 1..119: note = VH domain of 9359 IgG histidine scanning variant #2
EVQLVESGGGLVQPGGSLRLSCTASGHSLSSYAMGWVRQAPGKGLEWVGFINAGGNTYYANWAKGRFTISRDTSKNTVYLQMNSLRAEDTAVYFCARRYSRASGWYDLWGQGTLVTVSS

SEQ ID NO: 385 - protein, 119 aa, synthetic construct
  REGION 1..119: note = VH domain of 9359 IgG histidine scanning variant #3
EVQLVESGGGLVQPGGSLRLSCTASGFHLSSYAMGWVRQAPGKGLEWVGFINAGGNTYYANWAKGRFTISRDTSKNTVYLQMNSLRAEDTAVYFCARRYSRASGWYDLWGQGTLVTVSS

SEQ ID NO: 386 - protein, 119 aa, synthetic construct
  REGION 1..119: note = VH domain of 9359 IgG histidine scanning variant #4
EVQLVESGGGLVQPGGSLRLSCTASGFSHSSYAMGWVRQAPGKGLEWVGFINAGGNTYYANWAKGRFTISRDTSKNTVYLQMNSLRAEDTAVYFCARRYSRASGWYDLWGQGTLVTVSS

SEQ ID NO: 387 - protein, 119 aa, synthetic construct
  REGION 1..119: note = VH domain of 9359 IgG histidine scanning variant #5
EVQLVESGGGLVQPGGSLRLSCTASGFSLHSYAMGWVRQAPGKGLEWVGFINAGGNTYYANWAKGRFTISRDTSKNTVYLQMNSLRAEDTAVYFCARRYSRASGWYDLWGQGTLVTVSS

SEQ ID NO: 388 - protein, 119 aa, synthetic construct
  REGION 1..119: note = VH domain of 9359 IgG histidine scanning variant #6
EVQLVESGGGLVQPGGSLRLSCTASGFSLSHYAMGWVRQAPGKGLEWVGFINAGGNTYYANWAKGRFTISRDTSKNTVYLQMNSLRAEDTAVYFCARRYSRASGWYDLWGQGTLVTVSS

SEQ ID NO: 389 - protein, 119 aa, synthetic construct
  REGION 1..119: note = VH domain of 9359 IgG histidine scanning variant #7
EVQLVESGGGLVQPGGSLRLSCTASGFSLSSHAMGWVRQAPGKGLEWVGFINAGGNTYYANWAKGRFTISRDTSKNTVYLQMNSLRAEDTAVYFCARRYSRASGWYDLWGQGTLVTVSS

SEQ ID NO: 390 - protein, 119 aa, synthetic construct
  REGION 1..119: note = VH domain of 9359 IgG histidine scanning variant #8
EVQLVESGGGLVQPGGSLRLSCTASGFSLSSYHMGWVRQAPGKGLEWVGFINAGGNTYYANWAKGRFTISRDTSKNTVYLQMNSLRAEDTAVYFCARRYSRASGWYDLWGQGTLVTVSS

SEQ ID NO: 391 - protein, 119 aa, synthetic construct
  REGION 1..119: note = VH domain of 9359 IgG histidine scanning variant #9
EVQLVESGGGLVQPGGSLRLSCTASGFSLSSYAHGWVRQAPGKGLEWVGFINAGGNTYYANWAKGRFTISRDTSKNTVYLQMNSLRAEDTAVYFCARRYSRASGWYDLWGQGTLVTVSS

SEQ ID NO: 392 - protein, 119 aa, synthetic construct
  REGION 1..119: note = VH domain of 9359 IgG histidine scanning variant #10
EVQLVESGGGLVQPGGSLRLSCTASGFSLSSYAMHWVRQAPGKGLEWVGFINAGGNTYYANWAKGRFTISRDTSKNTVYLQMNSLRAEDTAVYFCARRYSRASGWYDLWGQGTLVTVSS

SEQ ID NO: 393 - protein, 119 aa, synthetic construct
  REGION 1..119: note = VH domain of 9359 IgG histidine scanning variant #11
EVQLVESGGGLVQPGGSLRLSCTASGFSLSSYAMGWVRQAPGKGLEWVGHINAGGNTYYANWAKGRFTISRDTSKNTVYLQMNSLRAEDTAVYFCARRYSRASGWYDLWGQGTLVTVSS

SEQ ID NO: 394 - protein, 119 aa, synthetic construct
  REGION 1..119: note = VH domain of 9359 IgG histidine scanning variant #12
EVQLVESGGGLVQPGGSLRLSCTASGFSLSSYAMGWVRQAPGKGLEWVGFHNAGGNTYYANWAKGRFTISRDTSKNTVYLQMNSLRAEDTAVYFCARRYSRASGWYDLWGQGTLVTVSS

SEQ ID NO: 395 - protein, 119 aa, synthetic construct
  REGION 1..119: note = VH domain of 9359 IgG histidine scanning variant #13
EVQLVESGGGLVQPGGSLRLSCTASGFSLSSYAMGWVRQAPGKGLEWVGFIHAGGNTYYANWAKGRFTISRDTSKNTVYLQMNSLRAEDTAVYFCARRYSRASGWYDLWGQGTLVTVSS

SEQ ID NO: 396 - protein, 119 aa, synthetic construct
  REGION 1..119: note = VH domain of 9359 IgG histidine scanning variant #14
EVQLVESGGGLVQPGGSLRLSCTASGFSLSSYAMGWVRQAPGKGLEWVGFINHGGNTYYANWAKGRFTISRDTSKNTVYLQMNSLRAEDTAVYFCARRYSRASGWYDLWGQGTLVTVSS

SEQ ID NO: 397 - protein, 119 aa, synthetic construct
  REGION 1..119: note = VH domain of 9359 IgG histidine scanning variant #15
EVQLVESGGGLVQPGGSLRLSCTASGFSLSSYAMGWVRQAPGKGLEWVGFINAHGNTYYANWAKGRFTISRDTSKNTVYLQMNSLRAEDTAVYFCARRYSRASGWYDLWGQGTLVTVSS

SEQ ID NO: 398 - protein, 119 aa, synthetic construct
  REGION 1..119: note = VH domain of 9359 IgG histidine scanning variant #16
EVQLVESGGGLVQPGGSLRLSCTASGFSLSSYAMGWVRQAPGKGLEWVGFINAGHNTYYANWAKGRFTISRDTSKNTVYLQMNSLRAEDTAVYFCARRYSRASGWYDLWGQGTLVTVSS

SEQ ID NO: 399 - protein, 119 aa, synthetic construct
  REGION 1..119: note = VH domain of 9359 IgG histidine scanning variant #17
EVQLVESGGGLVQPGGSLRLSCTASGFSLSSYAMGWVRQAPGKGLEWVGFINAGGHTYYANWAKGRFTISRDTSKNTVYLQMNSLRAEDTAVYFCARRYSRASGWYDLWGQGTLVTVSS

SEQ ID NO: 400 - protein, 119 aa, synthetic construct
  REGION 1..119: note = VH domain of 9359 IgG histidine scanning variant #18
EVQLVESGGGLVQPGGSLRLSCTASGFSLSSYAMGWVRQAPGKGLEWVGFINAGGNHYYANWAKGRFTISRDTSKNTVYLQMNSLRAEDTAVYFCARRYSRASGWYDLWGQGTLVTVSS

SEQ ID NO: 401 - protein, 119 aa, synthetic construct
  REGION 1..119: note = VH domain of 9359 IgG histidine scanning variant #19
EVQLVESGGGLVQPGGSLRLSCTASGFSLSSYAMGWVRQAPGKGLEWVGFINAGGNTHYANWAKGRFTISRDTSKNTVYLQMNSLRAEDTAVYFCARRYSRASGWYDLWGQGTLVTVSS

SEQ ID NO: 402 - protein, 119 aa, synthetic construct
  REGION 1..119: note = VH domain of 9359 IgG histidine scanning variant #20
EVQLVESGGGLVQPGGSLRLSCTASGFSLSSYAMGWVRQAPGKGLEWVGFINAGGNTYHANWAKGRFTISRDTSKNTVYLQMNSLRAEDTAVYFCARRYSRASGWYDLWGQGTLVTVSS

SEQ ID NO: 403 - protein, 119 aa, synthetic construct
  REGION 1..119: note = VH domain of 9359 IgG histidine scanning variant #21
EVQLVESGGGLVQPGGSLRLSCTASGFSLSSYAMGWVRQAPGKGLEWVGFINAGGNTYYHNWAKGRFTISRDTSKNTVYLQMNSLRAEDTAVYFCARRYSRASGWYDLWGQGTLVTVSS

SEQ ID NO: 404 - protein, 119 aa, synthetic construct
  REGION 1..119: note = VH domain of 9359 IgG histidine scanning variant #22
EVQLVESGGGLVQPGGSLRLSCTASGFSLSSYAMGWVRQAPGKGLEWVGFINAGGNTYYAHWAKGRFTISRDTSKNTVYLQMNSLRAEDTAVYFCARRYSRASGWYDLWGQGTLVTVSS

SEQ ID NO: 405 - protein, 119 aa, synthetic construct
  REGION 1..119: note = VH domain of 9359 IgG histidine scanning variant #23
EVQLVESGGGLVQPGGSLRLSCTASGFSLSSYAMGWVRQAPGKGLEWVGFINAGGNTYYANHAKGRFTISRDTSKNTVYLQMNSLRAEDTAVYFCARRYSRASGWYDLWGQGTLVTVSS

SEQ ID NO: 406 - protein, 119 aa, synthetic construct
  REGION 1..119: note = VH domain of 9359 IgG histidine scanning variant #24
EVQLVESGGGLVQPGGSLRLSCTASGFSLSSYAMGWVRQAPGKGLEWVGFINAGGNTYYANWHKGRFTISRDTSKNTVYLQMNSLRAEDTAVYFCARRYSRASGWYDLWGQGTLVTVSS

SEQ ID NO: 407 - protein, 119 aa, synthetic construct
  REGION 1..119: note = VH domain of 9359 IgG histidine scanning variant #25
EVQLVESGGGLVQPGGSLRLSCTASGFSLSSYAMGWVRQAPGKGLEWVGFINAGGNTYYANWAHGRFTISRDTSKNTVYLQMNSLRAEDTAVYFCARRYSRASGWYDLWGQGTLVTVSS

SEQ ID NO: 408 - protein, 119 aa, synthetic construct
  REGION 1..119: note = VH domain of 9359 IgG histidine scanning variant #26
EVQLVESGGGLVQPGGSLRLSCTASGFSLSSYAMGWVRQAPGKGLEWVGFINAGGNTYYANWAKHRFTISRDTSKNTVYLQMNSLRAEDTAVYFCARRYSRASGWYDLWGQGTLVTVSS

SEQ ID NO: 409 - protein, 119 aa, synthetic construct
  REGION 1..119: note = VH domain of 9359 IgG histidine scanning variant #27
EVQLVESGGGLVQPGGSLRLSCTASGFSLSSYAMGWVRQAPGKGLEWVGFINAGGNTYYANWAKGRFTISRDTSKNTVYLQMNSLRAEDTAVYFCHRRYSRASGWYDLWGQGTLVTVSS

SEQ ID NO: 410 - protein, 119 aa, synthetic construct
  REGION 1..119: note = VH domain of 9359 IgG histidine scanning variant #28
EVQLVESGGGLVQPGGSLRLSCTASGFSLSSYAMGWVRQAPGKGLEWVGFINAGGNTYYANWAKGRFTISRDTSKNTVYLQMNSLRAEDTAVYFCAHRYSRASGWYDLWGQGTLVTVSS

SEQ ID NO: 411 - protein, 119 aa, synthetic construct
  REGION 1..119: note = VH domain of 9359 IgG histidine scanning variant #29
EVQLVESGGGLVQPGGSLRLSCTASGFSLSSYAMGWVRQAPGKGLEWVGFINAGGNTYYANWAKGRFTISRDTSKNTVYLQMNSLRAEDTAVYFCARHYSRASGWYDLWGQGTLVTVSS

SEQ ID NO: 412 - protein, 119 aa, synthetic construct
  REGION 1..119: note = VH domain of 9359 IgG histidine scanning variant #30
EVQLVESGGGLVQPGGSLRLSCTASGFSLSSYAMGWVRQAPGKGLEWVGFINAGGNTYYANWAKGRFTISRDTSKNTVYLQMNSLRAEDTAVYFCARRHSRASGWYDLWGQGTLVTVSS

SEQ ID NO: 413 - protein, 119 aa, synthetic construct
  REGION 1..119: note = VH domain of 9359 IgG histidine scanning variant #31
EVQLVESGGGLVQPGGSLRLSCTASGFSLSSYAMGWVRQAPGKGLEWVGFINAGGNTYYANWAKGRFTISRDTSKNTVYLQMNSLRAEDTAVYFCARRYHRASGWYDLWGQGTLVTVSS

SEQ ID NO: 414 - protein, 119 aa, synthetic construct
  REGION 1..119: note = VH domain of 9359 IgG histidine scanning variant #32
EVQLVESGGGLVQPGGSLRLSCTASGFSLSSYAMGWVRQAPGKGLEWVGFINAGGNTYYANWAKGRFTISRDTSKNTVYLQMNSLRAEDTAVYFCARRYSHASGWYDLWGQGTLVTVSS

SEQ ID NO: 415 - protein, 119 aa, synthetic construct
  REGION 1..119: note = VH domain of 9359 IgG histidine scanning variant #33
EVQLVESGGGLVQPGGSLRLSCTASGFSLSSYAMGWVRQAPGKGLEWVGFINAGGNTYYANWAKGRFTISRDTSKNTVYLQMNSLRAEDTAVYFCARRYSRHSGWYDLWGQGTLVTVSS

SEQ ID NO: 416 - protein, 119 aa, synthetic construct
  REGION 1..119: note = VH domain of 9359 IgG histidine scanning variant #34
EVQLVESGGGLVQPGGSLRLSCTASGFSLSSYAMGWVRQAPGKGLEWVGFINAGGNTYYANWAKGRFTISRDTSKNTVYLQMNSLRAEDTAVYFCARRYSRAHGWYDLWGQGTLVTVSS

SEQ ID NO: 417 - protein, 119 aa, synthetic construct
  REGION 1..119: note = VH domain of 9359 IgG histidine scanning variant #35
EVQLVESGGGLVQPGGSLRLSCTASGFSLSSYAMGWVRQAPGKGLEWVGFINAGGNTYYANWAKGRFTISRDTSKNTVYLQMNSLRAEDTAVYFCARRYSRASHWYDLWGQGTLVTVSS

SEQ ID NO: 418 - protein, 119 aa, synthetic construct
  REGION 1..119: note = VH domain of 9359 IgG histidine scanning variant #36
EVQLVESGGGLVQPGGSLRLSCTASGFSLSSYAMGWVRQAPGKGLEWVGFINAGGNTYYANWAKGRFTISRDTSKNTVYLQMNSLRAEDTAVYFCARRYSRASGHYDLWGQGTLVTVSS

SEQ ID NO: 419 - protein, 119 aa, synthetic construct
  REGION 1..119: note = VH domain of 9359 IgG histidine scanning variant #37
EVQLVESGGGLVQPGGSLRLSCTASGFSLSSYAMGWVRQAPGKGLEWVGFINAGGNTYYANWAKGRFTISRDTSKNTVYLQMNSLRAEDTAVYFCARRYSRASGWHDLWGQGTLVTVSS

SEQ ID NO: 420 - protein, 119 aa, synthetic construct
  REGION 1..119: note = VH domain of 9359 IgG histidine scanning variant #38
EVQLVESGGGLVQPGGSLRLSCTASGFSLSSYAMGWVRQAPGKGLEWVGFINAGGNTYYANWAKGRFTISRDTSKNTVYLQMNSLRAEDTAVYFCARRYSRASGWYHLWGQGTLVTVSS

SEQ ID NO: 421 - protein, 119 aa, synthetic construct
  REGION 1..119: note = VH domain of 9359 IgG histidine scanning variant #39
EVQLVESGGGLVQPGGSLRLSCTASGFSLSSYAMGWVRQAPGKGLEWVGFINAGGNTYYANWAKGRFTISRDTSKNTVYLQMNSLRAEDTAVYFCARRYSRASGWYDHWGQGTLVTVSS

SEQ ID NO: 422 - protein, 119 aa, synthetic construct
  REGION 1..119: note = VH domain of 9359 IgG histidine scanning variant #40
EVQLVESGGGLVQPGGSLRLSCTASGFSLSSYAMGWVRQAPGKGLEWVGFINAGGNTYYANWAKGRFTISRDTSKNTVYLQMNSLRAEDTAVYYCARRYSRASGWYDLWGQGTLVTVSS

SEQ ID NO: 423 - protein, 119 aa, synthetic construct
  REGION 1..119: note = VH domain of 9359 IgG histidine scanning variant #41
EVQLVESGGGLVQPGGSLRLSCTASGFSLSSYAMGWVRQAPGKGLEWVGFINAGGNTYYANWAKGRFTISRDTSKNTVYLQMNSLRAEDTAVYFCARRYSRASGWYALWGQGTLVTVSS

SEQ ID NO: 424 - protein, 112 aa, synthetic construct
  REGION 1..112: note = VL domain of 9359 IgG histidine scanning variant #1
EIVMTQSPSTLSASVGDRVIITCHASQSISSYLAWYQQKPGKAPKLLIYAASTLASGVPSRFSGSGSGAEFTLTISSLQPDDFATYYCQSYSYSSGIDYGDVFGQGTKLTVL

SEQ ID NO: 425 - protein, 112 aa, synthetic construct
  REGION 1..112: note = VL domain of 9359 IgG histidine scanning variant #2
EIVMTQSPSTLSASVGDRVIITCQHSQSISSYLAWYQQKPGKAPKLLIYAASTLASGVPSRFSGSGSGAEFTLTISSLQPDDFATYYCQSYSYSSGIDYGDVFGQGTKLTVL

SEQ ID NO: 426 - protein, 112 aa, synthetic construct
  REGION 1..112: note = VL domain of 9359 IgG histidine scanning variant #3
EIVMTQSPSTLSASVGDRVIITCQAHQSISSYLAWYQQKPGKAPKLLIYAASTLASGVPSRFSGSGSGAEFTLTISSLQPDDFATYYCQSYSYSSGIDYGDVFGQGTKLTVL

SEQ ID NO: 427 - protein, 112 aa, synthetic construct
  REGION 1..112: note = VL domain of 9359 IgG histidine scanning variant #4
EIVMTQSPSTLSASVGDRVIITCQASHSISSYLAWYQQKPGKAPKLLIYAASTLASGVPSRFSGSGSGAEFTLTISSLQPDDFATYYCQSYSYSSGIDYGDVFGQGTKLTVL

SEQ ID NO: 428 - protein, 112 aa, synthetic construct
  REGION 1..112: note = VL domain of 9359 IgG histidine scanning variant #5
EIVMTQSPSTLSASVGDRVIITCQASQHISSYLAWYQQKPGKAPKLLIYAASTLASGVPSRFSGSGSGAEFTLTISSLQPDDFATYYCQSYSYSSGIDYGDVFGQGTKLTVL

SEQ ID NO: 429 - protein, 112 aa, synthetic construct
  REGION 1..112: note = VL domain of 9359 IgG histidine scanning variant #6
EIVMTQSPSTLSASVGDRVIITCQASQSHSSYLAWYQQKPGKAPKLLIYAASTLASGVPSRFSGSGSGAEFTLTISSLQPDDFATYYCQSYSYSSGIDYGDVFGQGTKLTVL

SEQ ID NO: 430 - protein, 112 aa, synthetic construct
  REGION 1..112: note = VL domain of 9359 IgG histidine scanning variant #7
EIVMTQSPSTLSASVGDRVIITCQASQSIHSYLAWYQQKPGKAPKLLIYAASTLASGVPSRFSGSGSGAEFTLTISSLQPDDFATYYCQSYSYSSGIDYGDVFGQGTKLTVL

SEQ ID NO: 431 - protein, 112 aa, synthetic construct
  REGION 1..112: note = VL domain of 9359 IgG histidine scanning variant #8
EIVMTQSPSTLSASVGDRVIITCQASQSISHYLAWYQQKPGKAPKLLIYAASTLASGVPSRFSGSGSGAEFTLTISSLQPDDFATYYCQSYSYSSGIDYGDVFGQGTKLTVL

SEQ ID NO: 432 - protein, 112 aa, synthetic construct
  REGION 1..112: note = VL domain of 9359 IgG histidine scanning variant #9
EIVMTQSPSTLSASVGDRVIITCQASQSISSHLAWYQQKPGKAPKLLIYAASTLASGVPSRFSGSGSGAEFTLTISSLQPDDFATYYCQSYSYSSGIDYGDVFGQGTKLTVL

SEQ ID NO: 433 - protein, 112 aa, synthetic construct
  REGION 1..112: note = VL domain of 9359 IgG histidine scanning variant #10
EIVMTQSPSTLSASVGDRVIITCQASQSISSYHAWYQQKPGKAPKLLIYAASTLASGVPSRFSGSGSGAEFTLTISSLQPDDFATYYCQSYSYSSGIDYGDVFGQGTKLTVL

SEQ ID NO: 434 - protein, 112 aa, synthetic construct
  REGION 1..112: note = VL domain of 9359 IgG histidine scanning variant #11
EIVMTQSPSTLSASVGDRVIITCQASQSISSYLHWYQQKPGKAPKLLIYAASTLASGVPSRFSGSGSGAEFTLTISSLQPDDFATYYCQSYSYSSGIDYGDVFGQGTKLTVL

SEQ ID NO: 435 - protein, 112 aa, synthetic construct
  REGION 1..112: note = VL domain of 9359 IgG histidine scanning variant #12
EIVMTQSPSTLSASVGDRVIITCQASQSISSYLAWYQQKPGKAPKLLIYHASTLASGVPSRFSGSGSGAEFTLTISSLQPDDFATYYCQSYSYSSGIDYGDVFGQGTKLTVL

SEQ ID NO: 436 - protein, 112 aa, synthetic construct
  REGION 1..112: note = VL domain of 9359 IgG histidine scanning variant #13
EIVMTQSPSTLSASVGDRVIITCQASQSISSYLAWYQQKPGKAPKLLIYAHSTLASGVPSRFSGSGSGAEFTLTISSLQPDDFATYYCQSYSYSSGIDYGDVFGQGTKLTVL

SEQ ID NO: 437 - protein, 112 aa, synthetic construct
  REGION 1..112: note = VL domain of 9359 IgG histidine scanning variant #14
EIVMTQSPSTLSASVGDRVIITCQASQSISSYLAWYQQKPGKAPKLLIYAAHTLASGVPSRFSGSGSGAEFTLTISSLQPDDFATYYCQSYSYSSGIDYGDVFGQGTKLTVL

SEQ ID NO: 438 - protein, 112 aa, synthetic construct
  REGION 1..112: note = VL domain of 9359 IgG histidine scanning variant #15
EIVMTQSPSTLSASVGDRVIITCQASQSISSYLAWYQQKPGKAPKLLIYAASHLASGVPSRFSGSGSGAEFTLTISSLQPDDFATYYCQSYSYSSGIDYGDVFGQGTKLTVL

SEQ ID NO: 439 - protein, 112 aa, synthetic construct
  REGION 1..112: note = VL domain of 9359 IgG histidine scanning variant #16
EIVMTQSPSTLSASVGDRVIITCQASQSISSYLAWYQQKPGKAPKLLIYAASTHASGVPSRFSGSGSGAEFTLTISSLQPDDFATYYCQSYSYSSGIDYGDVFGQGTKLTVL

SEQ ID NO: 440 - protein, 112 aa, synthetic construct
  REGION 1..112: note = VL domain of 9359 IgG histidine scanning variant #17
EIVMTQSPSTLSASVGDRVIITCQASQSISSYLAWYQQKPGKAPKLLIYAASTLHSGVPSRFSGSGSGAEFTLTISSLQPDDFATYYCQSYSYSSGIDYGDVFGQGTKLTVL

SEQ ID NO: 441 - protein, 112 aa, synthetic construct
  REGION 1..112: note = VL domain of 9359 IgG histidine scanning variant #18
EIVMTQSPSTLSASVGDRVIITCQASQSISSYLAWYQQKPGKAPKLLIYAASTLAHGVPSRFSGSGSGAEFTLTISSLQPDDFATYYCQSYSYSSGIDYGDVFGQGTKLTVL

SEQ ID NO: 442 - protein, 112 aa, synthetic construct
  REGION 1..112: note = VL domain of 9359 IgG histidine scanning variant #19
EIVMTQSPSTLSASVGDRVIITCQASQSISSYLAWYQQKPGKAPKLLIYAASTLASGVPSRFSGSGSGAEFTLTISSLQPDDFATYYCHSYSYSSGIDYGDVFGQGTKLTVL

SEQ ID NO: 443 - protein, 112 aa, synthetic construct
  REGION 1..112: note = VL domain of 9359 IgG histidine scanning variant #20
EIVMTQSPSTLSASVGDRVIITCQASQSISSYLAWYQQKPGKAPKLLIYAASTLASGVPSRFSGSGSGAEFTLTISSLQPDDFATYYCQHYSYSSGIDYGDVFGQGTKLTVL

SEQ ID NO: 444 - protein, 112 aa, synthetic construct
  REGION 1..112: note = VL domain of 9359 IgG histidine scanning variant #21
EIVMTQSPSTLSASVGDRVIITCQASQSISSYLAWYQQKPGKAPKLLIYAASTLASGVPSRFSGSGSGAEFTLTISSLQPDDFATYYCQSHSYSSGIDYGDVFGQGTKLTVL

SEQ ID NO: 445 - protein, 112 aa, synthetic construct
  REGION 1..112: note = VL domain of 9359 IgG histidine scanning variant #22
EIVMTQSPSTLSASVGDRVIITCQASQSISSYLAWYQQKPGKAPKLLIYAASTLASGVPSRFSGSGSGAEFTLTISSLQPDDFATYYCQSYHYSSGIDYGDVFGQGTKLTVL

SEQ ID NO: 446 - protein, 112 aa, synthetic construct
  REGION 1..112: note = VL domain of 9359 IgG histidine scanning variant #23
EIVMTQSPSTLSASVGDRVIITCQASQSISSYLAWYQQKPGKAPKLLIYAASTLASGVPSRFSGSGSGAEFTLTISSLQPDDFATYYCQSYSHSSGIDYGDVFGQGTKLTVL

SEQ ID NO: 447 - protein, 112 aa, synthetic construct
  REGION 1..112: note = VL domain of 9359 IgG histidine scanning variant #24
EIVMTQSPSTLSASVGDRVIITCQASQSISSYLAWYQQKPGKAPKLLIYAASTLASGVPSRFSGSGSGAEFTLTISSLQPDDFATYYCQSYSYHSGIDYGDVFGQGTKLTVL

SEQ ID NO: 448 - protein, 112 aa, synthetic construct
  REGION 1..112: note = VL domain of 9359 IgG histidine scanning variant #25
EIVMTQSPSTLSASVGDRVIITCQASQSISSYLAWYQQKPGKAPKLLIYAASTLASGVPSRFSGSGSGAEFTLTISSLQPDDFATYYCQSYSYSHGIDYGDVFGQGTKLTVL

SEQ ID NO: 449 - protein, 112 aa, synthetic construct
  REGION 1..112: note = VL domain of 9359 IgG histidine scanning variant #26
EIVMTQSPSTLSASVGDRVIITCQASQSISSYLAWYQQKPGKAPKLLIYAASTLASGVPSRFSGSGSGAEFTLTISSLQPDDFATYYCQSYSYSSHIDYGDVFGQGTKLTVL

SEQ ID NO: 450 - protein, 112 aa, synthetic construct
  REGION 1..112: note = VL domain of 9359 IgG histidine scanning variant #27
EIVMTQSPSTLSASVGDRVIITCQASQSISSYLAWYQQKPGKAPKLLIYAASTLASGVPSRFSGSGSGAEFTLTISSLQPDDFATYYCQSYSYSSGHDYGDVFGQGTKLTVL

SEQ ID NO: 451 - protein, 112 aa, synthetic construct
  REGION 1..112: note = VL domain of 9359 IgG histidine scanning variant #28
EIVMTQSPSTLSASVGDRVIITCQASQSISSYLAWYQQKPGKAPKLLIYAASTLASGVPSRFSGSGSGAEFTLTISSLQPDDFATYYCQSYSYSSGIHYGDVFGQGTKLTVL

SEQ ID NO: 452 - protein, 112 aa, synthetic construct
  REGION 1..112: note = VL domain of 9359 IgG histidine scanning variant #29
EIVMTQSPSTLSASVGDRVIITCQASQSISSYLAWYQQKPGKAPKLLIYAASTLASGVPSRFSGSGSGAEFTLTISSLQPDDFATYYCQSYSYSSGIDHGDVFGQGTKLTVL

SEQ ID NO: 453 - protein, 112 aa, synthetic construct
  REGION 1..112: note = VL domain of 9359 IgG histidine scanning variant #30
EIVMTQSPSTLSASVGDRVIITCQASQSISSYLAWYQQKPGKAPKLLIYAASTLASGVPSRFSGSGSGAEFTLTISSLQPDDFATYYCQSYSYSSGIDYHDVFGQGTKLTVL

SEQ ID NO: 454 - protein, 112 aa, synthetic construct
  REGION 1..112: note = VL domain of 9359 IgG histidine scanning variant #31
EIVMTQSPSTLSASVGDRVIITCQASQSISSYLAWYQQKPGKAPKLLIYAASTLASGVPSRFSGSGSGAEFTLTISSLQPDDFATYYCQSYSYSSGIDYGHVFGQGTKLTVL

SEQ ID NO: 455 - protein, 112 aa, synthetic construct
  REGION 1..112: note = VL domain of 9359 IgG histidine scanning variant #32
EIVMTQSPSTLSASVGDRVIITCQASQSISSYLAWYQQKPGKAPKLLIYAASTLASGVPSRFSGSGSGAEFTLTISSLQPDDFATYYCQSYSYSSGIDYGDHFGQGTKLTVL

SEQ ID NO: 456 - protein, 112 aa, synthetic construct
  REGION 1..112: note = VL domain of 9359 IgG histidine scanning variant #33
EIVMTQSPSTLSASVGDRVIITCQASQSISSYLAWYQQKPGKAPKLLIYAASTLASGVPSRFSGSGSGAEFTLTISSLQPDDFATYYCQSYSYSSGIDYGDVFGQGTKLEIK

SEQ ID NO: 457 - protein, 112 aa, synthetic construct
  REGION 1..112: note = VL domain of 9359 IgG histidine scanning variant #34
EIVMTQSPSTLSASVGDRVIITCQASQSISSYLAWYQQKPGKAPKLLIYAASTLASGVPSRFSGSGSGAEFTLTISSLQPDDFATYYCQSYSYSSGIAYGDVFGQGTKLTVL

SEQ ID NO: 458 - protein, 112 aa, synthetic construct
  REGION 1..112: note = VL domain of 9359 IgG histidine scanning variant #35
EIVMTQSPSTLSASVGDRVIITCQASQSISSYLAWYQQKPGKAPKLLIYAASTLASGVPSRFSGSGSGAEFTLTISSLQPDDFATYYCQSYSYSSGIDYGAVFGQGTKLTVL

SEQ ID NO: 459 - protein, 122 aa, synthetic construct
  REGION 1..122: note = VH domain of 9361 IgG
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARGDDGYTNAAYVSNLWGQGTLVTVSS

SEQ ID NO: 460 - protein, 108 aa, synthetic construct
  REGION 1..108: note = VL domain of 9361 IgG
EIVMTQSPSTLSASVGDRVIITCQASQSISSWLSWYQQKPGKAPKLLIYKASTLASGVPSRFSGSGSGAEFTLTISSLQPDDFATYYCQSYFDSTTIAFGQGTKLTVL

SEQ ID NO: 461 - protein, 10 aa, synthetic construct
  REGION 1..10: note = Heavy Chain CDR1 of 9361 IgG
GLSLSSYWMS

SEQ ID NO: 462 - protein, 16 aa, synthetic construct
  REGION 1..16: note = Heavy Chain CDR2 of 9361 IgG
VISAGGNTYYANWAKG

SEQ ID NO: 463 - protein, 14 aa, synthetic construct
  REGION 1..14: note = Heavy Chain CDR3 of 9361 IgG
GDDGYTNAAYVSNL

SEQ ID NO: 464 - protein, 11 aa, synthetic construct
  REGION 1..11: note = Light Chain CDR1 of 9361 IgG
QASQSISSWLS

SEQ ID NO: 465 - protein, 7 aa, synthetic construct
  REGION 1..7: note = Light Chain CDR2 of 9361 IgG
KASTLAS

SEQ ID NO: 466 - protein, 10 aa, synthetic construct
  REGION 1..10: note = Light Chain CDR3 of 9361 IgG
QSYFDSTTIA

SEQ ID NO: 467 - protein, 122 aa, synthetic construct
  REGION 1..122: note = VH domain of 9361 IgG histidine scanning variant #1
EVQLVESGGGLVQPGGSLRLSCTASHLSLSSYWMSWVRQAPGKGLEWVGVISAGGNTYYANWAKGRFTISRDTSKNTVYLQMNSLRAEDTAVYFCARGDDGYTNAAYVSNLWGQGTLVTVSS

SEQ ID NO: 468 - protein, 122 aa, synthetic construct
  REGION 1..122: note = VH domain of 9361 IgG histidine scanning variant #2
EVQLVESGGGLVQPGGSLRLSCTASGHSLSSYWMSWVRQAPGKGLEWVGVISAGGNTYYANWAKGRFTISRDTSKNTVYLQMNSLRAEDTAVYFCARGDDGYTNAAYVSNLWGQGTLVTVSS

SEQ ID NO: 469 - protein, 122 aa, synthetic construct
  REGION 1..122: note = VH domain of 9361 IgG histidine scanning variant #3
EVQLVESGGGLVQPGGSLRLSCTASGLHLSSYWMSWVRQAPGKGLEWVGVISAGGNTYYANWAKGRFTISRDTSKNTVYLQMNSLRAEDTAVYFCARGDDGYTNAAYVSNLWGQGTLVTVSS

SEQ ID NO: 470 - protein, 122 aa, synthetic construct
  REGION 1..122: note = VH domain of 9361 IgG histidine scanning variant #4
EVQLVESGGGLVQPGGSLRLSCTASGLSHSSYWMSWVRQAPGKGLEWVGVISAGGNTYYANWAKGRFTISRDTSKNTVYLQMNSLRAEDTAVYFCARGDDGYTNAAYVSNLWGQGTLVTVSS

SEQ ID NO: 471 - protein, 122 aa, synthetic construct
  REGION 1..122: note = VH domain of 9361 IgG histidine scanning variant #5
EVQLVESGGGLVQPGGSLRLSCTASGLSLHSYWMSWVRQAPGKGLEWVGVISAGGNTYYANWAKGRFTISRDTSKNTVYLQMNSLRAEDTAVYFCARGDDGYTNAAYVSNLWGQGTLVTVSS

SEQ ID NO: 472 - protein, 122 aa, synthetic construct
  REGION 1..122: note = VH domain of 9361 IgG histidine scanning variant #6
EVQLVESGGGLVQPGGSLRLSCTASGLSLSHYWMSWVRQAPGKGLEWVGVISAGGNTYYANWAKGRFTISRDTSKNTVYLQMNSLRAEDTAVYFCARGDDGYTNAAYVSNLWGQGTLVTVSS

SEQ ID NO: 473 - protein, 122 aa, synthetic construct
  REGION 1..122: note = VH domain of 9361 IgG histidine scanning variant #7
EVQLVESGGGLVQPGGSLRLSCTASGLSLSSHWMSWVRQAPGKGLEWVGVISAGGNTYYANWAKGRFTISRDTSKNTVYLQMNSLRAEDTAVYFCARGDDGYTNAAYVSNLWGQGTLVTVSS

SEQ ID NO: 474 - protein, 122 aa, synthetic construct
  REGION 1..122: note = VH domain of 9361 IgG histidine scanning variant #8
EVQLVESGGGLVQPGGSLRLSCTASGLSLSSYHMSWVRQAPGKGLEWVGVISAGGNTYYANWAKGRFTISRDTSKNTVYLQMNSLRAEDTAVYFCARGDDGYTNAAYVSNLWGQGTLVTVSS

SEQ ID NO: 475 - protein, 122 aa, synthetic construct
  REGION 1..122: note = VH domain of 9361 IgG histidine scanning variant #9
EVQLVESGGGLVQPGGSLRLSCTASGLSLSSYWHSWVRQAPGKGLEWVGVISAGGNTYYANWAKGRFTISRDTSKNTVYLQMNSLRAEDTAVYFCARGDDGYTNAAYVSNLWGQGTLVTVSS

SEQ ID NO: 476 - protein, 122 aa, synthetic construct
  REGION 1..122: note = VH domain of 9361 IgG histidine scanning variant #10
EVQLVESGGGLVQPGGSLRLSCTASGLSLSSYWMHWVRQAPGKGLEWVGVISAGGNTYYANWAKGRFTISRDTSKNTVYLQMNSLRAEDTAVYFCARGDDGYTNAAYVSNLWGQGTLVTVSS

SEQ ID NO: 477 - protein, 122 aa, synthetic construct
  REGION 1..122: note = VH domain of 9361 IgG histidine scanning variant #11
EVQLVESGGGLVQPGGSLRLSCTASGLSLSSYWMSWVRQAPGKGLEWVGHISAGGNTYYANWAKGRFTISRDTSKNTVYLQMNSLRAEDTAVYFCARGDDGYTNAAYVSNLWGQGTLVTVSS

SEQ ID NO: 478 - protein, 122 aa, synthetic construct
  REGION 1..122: note = VH domain of 9361 IgG histidine scanning variant #12
EVQLVESGGGLVQPGGSLRLSCTASGLSLSSYWMSWVRQAPGKGLEWVGVHSAGGNTYYANWAKGRFTISRDTSKNTVYLQMNSLRAEDTAVYFCARGDDGYTNAAYVSNLWGQGTLVTVSS

SEQ ID NO: 479 - protein, 122 aa, synthetic construct
  REGION 1..122: note = VH domain of 9361 IgG histidine scanning variant #13
EVQLVESGGGLVQPGGSLRLSCTASGLSLSSYWMSWVRQAPGKGLEWVGVIHAGGNTYYANWAKGRFTISRDTSKNTVYLQMNSLRAEDTAVYFCARGDDGYTNAAYVSNLWGQGTLVTVSS

SEQ ID NO: 480 - protein, 122 aa, synthetic construct
  REGION 1..122: note = VH domain of 9361 IgG histidine scanning variant #14
EVQLVESGGGLVQPGGSLRLSCTASGLSLSSYWMSWVRQAPGKGLEWVGVISHGGNTYYANWAKGRFTISRDTSKNTVYLQMNSLRAEDTAVYFCARGDDGYTNAAYVSNLWGQGTLVTVSS

SEQ ID NO: 481 - protein, 122 aa, synthetic construct
  REGION 1..122: note = VH domain of 9361 IgG histidine scanning variant #15
EVQLVESGGGLVQPGGSLRLSCTASGLSLSSYWMSWVRQAPGKGLEWVGVISAHGNTYYANWAKGRFTISRDTSKNTVYLQMNSLRAEDTAVYFCARGDDGYTNAAYVSNLWGQGTLVTVSS

SEQ ID NO: 482 - protein, 122 aa, synthetic construct
  REGION 1..122: note = VH domain of 9361 IgG histidine scanning variant #16
EVQLVESGGGLVQPGGSLRLSCTASGLSLSSYWMSWVRQAPGKGLEWVGVISAGHNTYYANWAKGRFTISRDTSKNTVYLQMNSLRAEDTAVYFCARGDDGYTNAAYVSNLWGQGTLVTVSS

SEQ ID NO: 483 - protein, 122 aa, synthetic construct
  REGION 1..122: note = VH domain of 9361 IgG histidine scanning variant #17
EVQLVESGGGLVQPGGSLRLSCTASGLSLSSYWMSWVRQAPGKGLEWVGVISAGGHTYYANWAKGRFTISRDTSKNTVYLQMNSLRAEDTAVYFCARGDDGYTNAAYVSNLWGQGTLVTVSS

SEQ ID NO: 484 - protein, 122 aa, synthetic construct
  REGION 1..122: note = VH domain of 9361 IgG histidine scanning variant #18
EVQLVESGGGLVQPGGSLRLSCTASGLSLSSYWMSWVRQAPGKGLEWVGVISAGGNHYYANWAKGRFTISRDTSKNTVYLQMNSLRAEDTAVYFCARGDDGYTNAAYVSNLWGQGTLVTVSS

SEQ ID NO: 485 - protein, 122 aa, synthetic construct
  REGION 1..122: note = VH domain of 9361 IgG histidine scanning variant #19
EVQLVESGGGLVQPGGSLRLSCTASGLSLSSYWMSWVRQAPGKGLEWVGVISAGGNTHYANWAKGRFTISRDTSKNTVYLQMNSLRAEDTAVYFCARGDDGYTNAAYVSNLWGQGTLVTVSS

SEQ ID NO: 486 - protein, 122 aa, synthetic construct
  REGION 1..122: note = VH domain of 9361 IgG histidine scanning variant #20
EVQLVESGGGLVQPGGSLRLSCTASGLSLSSYWMSWVRQAPGKGLEWVGVISAGGNTYHANWAKGRFTISRDTSKNTVYLQMNSLRAEDTAVYFCARGDDGYTNAAYVSNLWGQGTLVTVSS

SEQ ID NO: 487 - protein, 122 aa, synthetic construct
  REGION 1..122: note = VH domain of 9361 IgG histidine scanning variant #21
EVQLVESGGGLVQPGGSLRLSCTASGLSLSSYWMSWVRQAPGKGLEWVGVISAGGNTYYHNWAKGRFTISRDTSKNTVYLQMNSLRAEDTAVYFCARGDDGYTNAAYVSNLWGQGTLVTVSS

SEQ ID NO: 488 - protein, 122 aa, synthetic construct
  REGION 1..122: note = VH domain of 9361 IgG histidine scanning variant #22
EVQLVESGGGLVQPGGSLRLSCTASGLSLSSYWMSWVRQAPGKGLEWVGVISAGGNTYYAHWAKGRFTISRDTSKNTVYLQMNSLRAEDTAVYFCARGDDGYTNAAYVSNLWGQGTLVTVSS

SEQ ID NO: 489 - protein, 122 aa, synthetic construct
  REGION 1..122: note = VH domain of 9361 IgG histidine scanning variant #23
EVQLVESGGGLVQPGGSLRLSCTASGLSLSSYWMSWVRQAPGKGLEWVGVISAGGNTYYANHAKGRFTISRDTSKNTVYLQMNSLRAEDTAVYFCARGDDGYTNAAYVSNLWGQGTLVTVSS

SEQ ID NO: 490 - protein, 122 aa, synthetic construct
  REGION 1..122: note = VH domain of 9361 IgG histidine scanning variant #24
EVQLVESGGGLVQPGGSLRLSCTASGLSLSSYWMSWVRQAPGKGLEWVGVISAGGNTYYANWHKGRFTISRDTSKNTVYLQMNSLRAEDTAVYFCARGDDGYTNAAYVSNLWGQGTLVTVSS

SEQ ID NO: 491 - protein, 122 aa, synthetic construct
  REGION 1..122: note = VH domain of 9361 IgG histidine scanning variant #25
EVQLVESGGGLVQPGGSLRLSCTASGLSLSSYWMSWVRQAPGKGLEWVGVISAGGNTYYANWAHGRFTISRDTSKNTVYLQMNSLRAEDTAVYFCARGDDGYTNAAYVSNLWGQGTLVTVSS

SEQ ID NO: 492 - protein, 122 aa, synthetic construct
  REGION 1..122: note = VH domain of 9361 IgG histidine scanning variant #26
EVQLVESGGGLVQPGGSLRLSCTASGLSLSSYWMSWVRQAPGKGLEWVGVISAGGNTYYANWAKHRFTISRDTSKNTVYLQMNSLRAEDTAVYFCARGDDGYTNAAYVSNLWGQGTLVTVSS

SEQ ID NO: 493 - protein, 122 aa, synthetic construct
  REGION 1..122: note = VH domain of 9361 IgG histidine scanning variant #27
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCHRGDDGYTNAAYVSNLWGQGTLVTVSS

SEQ ID NO: 494 - protein, 122 aa, synthetic construct
  REGION 1..122: note = VH domain of 9361 IgG histidine scanning variant #28
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCAHGDDGYTNAAYVSNLWGQGTLVTVSS

SEQ ID NO: 495 - protein, 122 aa, synthetic construct
  REGION 1..122: note = VH domain of 9361 IgG histidine scanning variant #29
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARHDDGYTNAAYVSNLWGQGTLVTVSS

SEQ ID NO: 496 - protein, 122 aa, synthetic construct
  REGION 1..122: note = VH domain of 9361 IgG histidine scanning variant #30
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARGHDGYTNAAYVSNLWGQGTLVTVSS

SEQ ID NO: 497 - protein, 122 aa, synthetic construct
  REGION 1..122: note = VH domain of 9361 IgG histidine scanning variant #31
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARGDHGYTNAAYVSNLWGQGTLVTVSS

SEQ ID NO: 498 - protein, 122 aa, synthetic construct
  REGION 1..122: note = VH domain of 9361 IgG histidine scanning variant #32
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARGDDHYTNAAYVSNLWGQGTLVTVSS

SEQ ID NO: 499 - protein, 122 aa, synthetic construct
  REGION 1..122: note = VH domain of 9361 IgG histidine scanning variant #33
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARGDDGHTNAAYVSNLWGQGTLVTVSS

SEQ ID NO: 500 - protein, 122 aa, synthetic construct
  REGION 1..122: note = VH domain of 9361 IgG histidine scanning variant #34
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARGDDGYHNAAYVSNLWGQGTLVTVSS

SEQ ID NO: 501 - protein, 122 aa, synthetic construct
  REGION 1..122: note = VH domain of 9361 IgG histidine scanning variant #35
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARGDDGYTHAAYVSNLWGQGTLVTVSS

SEQ ID NO: 502 - protein, 122 aa, synthetic construct
  REGION 1..122: note = VH domain of 9361 IgG histidine scanning variant #36
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARGDDGYTNHAYVSNLWGQGTLVTVSS

SEQ ID NO: 503 - protein, 122 aa, synthetic construct
  REGION 1..122: note = VH domain of 9361 IgG histidine scanning variant #37
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARGDDGYTNAHYVSNLWGQGTLVTVSS

SEQ ID NO: 504 - protein, 122 aa, synthetic construct
  REGION 1..122: note = VH domain of 9361 IgG histidine scanning variant #38
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARGDDGYTNAAHVSNLWGQGTLVTVSS

SEQ ID NO: 505 - protein, 122 aa, synthetic construct
  REGION 1..122: note = VH domain of 9361 IgG histidine scanning variant #39
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARGDDGYTNAAYHSNLWGQGTLVTVSS

SEQ ID NO: 506 - protein, 122 aa, synthetic construct
  REGION 1..122: note = VH domain of 9361 IgG histidine scanning variant #40
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARGDDGYTNAAYVHNLWGQGTLVTVSS

SEQ ID NO: 507 - protein, 122 aa, synthetic construct
  REGION 1..122: note = VH domain of 9361 IgG histidine scanning variant #41
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARGDDGYTNAAYVSHLWGQGTLVTVSS

SEQ ID NO: 508 - protein, 122 aa, synthetic construct
  REGION 1..122: note = VH domain of 9361 IgG histidine scanning variant #42
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARGDDGYTNAAYVSNHWGQGTLVTVSS

SEQ ID NO: 509 - protein, 122 aa, synthetic construct
  REGION 1..122: note = VH domain of 9361 IgG histidine scanning variant #43
EVQLVESGGGLVQPGGSLRLSCTASGLSLSSYWMSWVRQAPGKGLEWVGVISAGGNTYYANWAKGRFTISRDTSKNTVYLQMNSLRAEDTAVYYCARGDDGYTNAAYVSNLWGQGTLVTVSS

SEQ ID NO: 510 - protein, 122 aa, synthetic construct
  REGION 1..122: note = VH domain of 9361 IgG histidine scanning variant #44
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARGADGYTNAAYVSNLWGQGTLVTVSS

SEQ ID NO: 511 - protein, 122 aa, synthetic construct
  REGION 1..122: note = VH domain of 9361 IgG histidine scanning variant #45
EVQLVESGGGLVQPGGSLRLSCTASGLSLSSYWMSWVRQAPGKGLEWVGVISAGGNTYYANWAKGRFTISRDTSKNTVYLQMNSLRAEDTAVYFCARGDAGYTNAAYVSNLWGQGTLVTVSS

SEQ ID NO: 512 - protein, 108 aa, synthetic construct
  REGION 1..108: note = VL domain of 9361 IgG histidine scanning variant #1
EIVMTQSPSTLSASVGDRVIITCHASQSISSWLSWYQQKPGKAPKLLIYKASTLASGVPSRFSGSGSGAEFTLTISSLQPDDFATYYCQSYFDSTTIAFGQGTKLTVL

SEQ ID NO: 513 - protein, 108 aa, synthetic construct
  REGION 1..108: note = VL domain of 9361 IgG histidine scanning variant #2
EIVMTQSPSTLSASVGDRVIITCQHSQSISSWLSWYQQKPGKAPKLLIYKASTLASGVPSRFSGSGSGAEFTLTISSLQPDDFATYYCQSYFDSTTIAFGQGTKLTVL

SEQ ID NO: 514 - protein, 108 aa, synthetic construct
  REGION 1..108: note = VL domain of 9361 IgG histidine scanning variant #3
EIVMTQSPSTLSASVGDRVIITCQAHQSISSWLSWYQQKPGKAPKLLIYKASTLASGVPSRFSGSGSGAEFTLTISSLQPDDFATYYCQSYFDSTTIAFGQGTKLTVL

SEQ ID NO: 515 - protein, 108 aa, synthetic construct
  REGION 1..108: note = VL domain of 9361 IgG histidine scanning variant #4
EIVMTQSPSTLSASVGDRVIITCQASHSISSWLSWYQQKPGKAPKLLIYKASTLASGVPSRFSGSGSGAEFTLTISSLQPDDFATYYCQSYFDSTTIAFGQGTKLTVL

SEQ ID NO: 516 - protein, 108 aa, synthetic construct
  REGION 1..108: note = VL domain of 9361 IgG histidine scanning variant #5
EIVMTQSPSTLSASVGDRVIITCQASQHISSWLSWYQQKPGKAPKLLIYKASTLASGVPSRFSGSGSGAEFTLTISSLQPDDFATYYCQSYFDSTTIAFGQGTKLTVL

SEQ ID NO: 517 - protein, 108 aa, synthetic construct
  REGION 1..108: note = VL domain of 9361 IgG histidine scanning variant #6
EIVMTQSPSTLSASVGDRVIITCQASQSHSSWLSWYQQKPGKAPKLLIYKASTLASGVPSRFSGSGSGAEFTLTISSLQPDDFATYYCQSYFDSTTIAFGQGTKLTVL

SEQ ID NO: 518 - protein, 108 aa, synthetic construct
  REGION 1..108: note = VL domain of 9361 IgG histidine scanning variant #7
EIVMTQSPSTLSASVGDRVIITCQASQSIHSWLSWYQQKPGKAPKLLIYKASTLASGVPSRFSGSGSGAEFTLTISSLQPDDFATYYCQSYFDSTTIAFGQGTKLTVL

SEQ ID NO: 519 - protein, 108 aa, synthetic construct
  REGION 1..108: note = VL domain of 9361 IgG histidine scanning variant #8
EIVMTQSPSTLSASVGDRVIITCQASQSISHWLSWYQQKPGKAPKLLIYKASTLASGVPSRFSGSGSGAEFTLTISSLQPDDFATYYCQSYFDSTTIAFGQGTKLTVL

SEQ ID NO: 520 - protein, 108 aa, synthetic construct
  REGION 1..108: note = VL domain of 9361 IgG histidine scanning variant #9
EIVMTQSPSTLSASVGDRVIITCQASQSISSHLSWYQQKPGKAPKLLIYKASTLASGVPSRFSGSGSGAEFTLTISSLQPDDFATYYCQSYFDSTTIAFGQGTKLTVL

SEQ ID NO: 521 - protein, 108 aa, synthetic construct
  REGION 1..108: note = VL domain of 9361 IgG histidine scanning variant #10
EIVMTQSPSTLSASVGDRVIITCQASQSISSWHSWYQQKPGKAPKLLIYKASTLASGVPSRFSGSGSGAEFTLTISSLQPDDFATYYCQSYFDSTTIAFGQGTKLTVL

SEQ ID NO: 522 - protein, 108 aa, synthetic construct
  REGION 1..108: note = VL domain of 9361 IgG histidine scanning variant #11
EIVMTQSPSTLSASVGDRVIITCQASQSISSWLHWYQQKPGKAPKLLIYKASTLASGVPSRFSGSGSGAEFTLTISSLQPDDFATYYCQSYFDSTTIAFGQGTKLTVL

SEQ ID NO: 523 - protein, 108 aa, synthetic construct
  REGION 1..108: note = VL domain of 9361 IgG histidine scanning variant #12
EIVMTQSPSTLSASVGDRVIITCQASQSISSWLSWYQQKPGKAPKLLIYHASTLASGVPSRFSGSGSGAEFTLTISSLQPDDFATYYCQSYFDSTTIAFGQGTKLTVL

SEQ ID NO: 524 - protein, 108 aa, synthetic construct
  REGION 1..108: note = VL domain of 9361 IgG histidine scanning variant #13
EIVMTQSPSTLSASVGDRVIITCQASQSISSWLSWYQQKPGKAPKLLIYKHSTLASGVPSRFSGSGSGAEFTLTISSLQPDDFATYYCQSYFDSTTIAFGQGTKLTVL

SEQ ID NO: 525 - protein, 108 aa, synthetic construct
  REGION 1..108: note = VL domain of 9361 IgG histidine scanning variant #14
EIVMTQSPSTLSASVGDRVIITCQASQSISSWLSWYQQKPGKAPKLLIYKAHTLASGVPSRFSGSGSGAEFTLTISSLQPDDFATYYCQSYFDSTTIAFGQGTKLTVL

SEQ ID NO: 526 - protein, 108 aa, synthetic construct
  REGION 1..108: note = VL domain of 9361 IgG histidine scanning variant #15
EIVMTQSPSTLSASVGDRVIITCQASQSISSWLSWYQQKPGKAPKLLIYKASHLASGVPSRFSGSGSGAEFTLTISSLQPDDFATYYCQSYFDSTTIAFGQGTKLTVL

SEQ ID NO: 527 - protein, 108 aa, synthetic construct
  REGION 1..108: note = VL domain of 9361 IgG histidine scanning variant #16
EIVMTQSPSTLSASVGDRVIITCQASQSISSWLSWYQQKPGKAPKLLIYKASTHASGVPSRFSGSGSGAEFTLTISSLQPDDFATYYCQSYFDSTTIAFGQGTKLTVL

SEQ ID NO: 528 - protein, 108 aa, synthetic construct
  REGION 1..108: note = VL domain of 9361 IgG histidine scanning variant #17
EIVMTQSPSTLSASVGDRVIITCQASQSISSWLSWYQQKPGKAPKLLIYKASTLHSGVPSRFSGSGSGAEFTLTISSLQPDDFATYYCQSYFDSTTIAFGQGTKLTVL

SEQ ID NO: 529 - protein, 108 aa, synthetic construct
  REGION 1..108: note = VL domain of 9361 IgG histidine scanning variant #18
EIVMTQSPSTLSASVGDRVIITCQASQSISSWLSWYQQKPGKAPKLLIYKASTLAHGVPSRFSGSGSGAEFTLTISSLQPDDFATYYCQSYFDSTTIAFGQGTKLTVL

SEQ ID NO: 530 - protein, 108 aa, synthetic construct
  REGION 1..108: note = VL domain of 9361 IgG histidine scanning variant #19
EIVMTQSPSTLSASVGDRVIITCQASQSISSWLSWYQQKPGKAPKLLIYKASTLASGVPSRFSGSGSGAEFTLTISSLQPDDFATYYCHSYFDSTTIAFGQGTKLTVL

SEQ ID NO: 531 - protein, 108 aa, synthetic construct
  REGION 1..108: note = VL domain of 9361 IgG histidine scanning variant #20
EIVMTQSPSTLSASVGDRVIITCQASQSISSWLSWYQQKPGKAPKLLIYKASTLASGVPSRFSGSGSGAEFTLTISSLQPDDFATYYCQHYFDSTTIAFGQGTKLTVL

SEQ ID NO: 532 - protein, 108 aa, synthetic construct
  REGION 1..108: note = VL domain of 9361 IgG histidine scanning variant #21
EIVMTQSPSTLSASVGDRVIITCQASQSISSWLSWYQQKPGKAPKLLIYKASTLASGVPSRFSGSGSGAEFTLTISSLQPDDFATYYCQSHFDSTTIAFGQGTKLTVL

SEQ ID NO: 533 - protein, 108 aa, synthetic construct
  REGION 1..108: note = VL domain of 9361 IgG histidine scanning variant #22
EIVMTQSPSTLSASVGDRVIITCQASQSISSWLSWYQQKPGKAPKLLIYKASTLASGVPSRFSGSGSGAEFTLTISSLQPDDFATYYCQSYHDSTTIAFGQGTKLTVL

SEQ ID NO: 534 - protein, 108 aa, synthetic construct
  REGION 1..108: note = VL domain of 9361 IgG histidine scanning variant #23
EIVMTQSPSTLSASVGDRVIITCQASQSISSWLSWYQQKPGKAPKLLIYKASTLASGVPSRFSGSGSGAEFTLTISSLQPDDFATYYCQSYFHSTTIAFGQGTKLTVL

SEQ ID NO: 535 - protein, 108 aa, synthetic construct
  REGION 1..108: note = VL domain of 9361 IgG histidine scanning variant #24
EIVMTQSPSTLSASVGDRVIITCQASQSISSWLSWYQQKPGKAPKLLIYKASTLASGVPSRFSGSGSGAEFTLTISSLQPDDFATYYCQSYFDHTTIAFGQGTKLTVL

SEQ ID NO: 536 - protein, 108 aa, synthetic construct
  REGION 1..108: note = VL domain of 9361 IgG histidine scanning variant #25
EIVMTQSPSTLSASVGDRVIITCQASQSISSWLSWYQQKPGKAPKLLIYKASTLASGVPSRFSGSGSGAEFTLTISSLQPDDFATYYCQSYFDSHTIAFGQGTKLTVL

SEQ ID NO: 537 - protein, 108 aa, synthetic construct
  REGION 1..108: note = VL domain of 9361 IgG histidine scanning variant #26
EIVMTQSPSTLSASVGDRVIITCQASQSISSWLSWYQQKPGKAPKLLIYKASTLASGVPSRFSGSGSGAEFTLTISSLQPDDFATYYCQSYFDSTHIAFGQGTKLTVL

SEQ ID NO: 538 - protein, 108 aa, synthetic construct
  REGION 1..108: note = VL domain of 9361 IgG histidine scanning variant #27
EIVMTQSPSTLSASVGDRVIITCQASQSISSWLSWYQQKPGKAPKLLIYKASTLASGVPSRFSGSGSGAEFTLTISSLQPDDFATYYCQSYFDSTTHAFGQGTKLTVL

SEQ ID NO: 539 - protein, 108 aa, synthetic construct
  REGION 1..108: note = VL domain of 9361 IgG histidine scanning variant #28
EIVMTQSPSTLSASVGDRVIITCQASQSISSWLSWYQQKPGKAPKLLIYKASTLASGVPSRFSGSGSGAEFTLTISSLQPDDFATYYCQSYFDSTTIHFGQGTKLTVL

SEQ ID NO: 540 - protein, 108 aa, synthetic construct
  REGION 1..108: note = VL domain of 9361 IgG histidine scanning variant #29
EIVMTQSPSTLSASVGDRVIITCQASQSISSWLSWYQQKPGKAPKLLIYKASTLASGVPSRFSGSGSGAEFTLTISSLQPDDFATYYCQSYFDSTTIAFGQGTKLEIK

SEQ ID NO: 541 - protein, 108 aa, synthetic construct
  REGION 1..108: note = VL domain of 9361 IgG histidine scanning variant #30
EIVMTQSPSTLSASVGDRVIITCQASQSISSWLSWYQQKPGKAPKLLIYKASTLASGVPSRFSGSGSGAEFTLTISSLQPDDFATYYCQSYFASTTIAFGQGTKLTVL

SEQ ID NO: 542 - protein, 120 aa, synthetic construct
  REGION 1..120: note = VH domain of 9412 IgG
EVQLVESGGGLVQPGGSLRLSCTASGFSLSTYAMSWVRQAPGKGLEWVGMIYINDNTYYASWAKGRFTISRDTSKNTVYLQMNSLRAEDTAVYFCARDIGISYYIGVSLWGQGTLVTVSS

SEQ ID NO: 543 - protein, 111 aa, synthetic construct
  REGION 1..111: note = VL domain of 9412 IgG
EIVMTQSPSTLSASVGDRVIITCQASQSIGTRLSWYQQKPGKAPKLLIYGASKLATGVPSRFSGSGSGAEFTLTISSLQPDDFATYYCLGEYSYTTTDGIAFGQGTKLTVL

SEQ ID NO: 544 - protein, 10 aa, synthetic construct
  REGION 1..10: note = Heavy Chain CDR1 of 9412 IgG
GFSLSTYAMS

SEQ ID NO: 545 - protein, 16 aa, synthetic construct
  REGION 1..16: note = Heavy Chain CDR2 of 9412 IgG
MIYINDNTYYASWAKG

SEQ ID NO: 546 - protein, 14 aa, synthetic construct
  REGION 1..14: note = Heavy Chain CDR3 of 9412 IgG
ARDIGISYYIGVSL

SEQ ID NO: 547 - protein, 11 aa, synthetic construct
  REGION 1..11: note = Light Chain CDR1 of 9412 IgG
QASQSIGTRLS

SEQ ID NO: 548 - protein, 7 aa, synthetic construct
  REGION 1..7: note = Light Chain CDR2 of 9412 IgG
GASKLAT

SEQ ID NO: 549 - protein, 13 aa, synthetic construct
  REGION 1..13: note = Light Chain CDR3 of 9412 IgG
LGEYSYTTTDGIA

SEQ ID NO: 550 - protein, 120 aa, synthetic construct
  REGION 1..120: note = VH domain of 9412 IgG histidine scanning variant #1
EVQLVESGGGLVQPGGSLRLSCTASHFSLSTYAMSWVRQAPGKGLEWVGMIYINDNTYYASWAKGRFTISRDTSKNTVYLQMNSLRAEDTAVYFCARDIGISYYIGVSLWGQGTLVTVSS

SEQ ID NO: 551 - protein, 120 aa, synthetic construct
  REGION 1..120: note = VH domain of 9412 IgG histidine scanning variant #2
EVQLVESGGGLVQPGGSLRLSCTASGHSLSTYAMSWVRQAPGKGLEWVGMIYINDNTYYASWAKGRFTISRDTSKNTVYLQMNSLRAEDTAVYFCARDIGISYYIGVSLWGQGTLVTVSS

SEQ ID NO: 552 - protein, 120 aa, synthetic construct
  REGION 1..120: note = VH domain of 9412 IgG histidine scanning variant #3
EVQLVESGGGLVQPGGSLRLSCTASGFHLSTYAMSWVRQAPGKGLEWVGMIYINDNTYYASWAKGRFTISRDTSKNTVYLQMNSLRAEDTAVYFCARDIGISYYIGVSLWGQGTLVTVSS

SEQ ID NO: 553 - protein, 120 aa, synthetic construct
  REGION 1..120: note = VH domain of 9412 IgG histidine scanning variant #4
EVQLVESGGGLVQPGGSLRLSCTASGFSHSTYAMSWVRQAPGKGLEWVGMIYINDNTYYASWAKGRFTISRDTSKNTVYLQMNSLRAEDTAVYFCARDIGISYYIGVSLWGQGTLVTVSS

SEQ ID NO: 554 - protein, 120 aa, synthetic construct
  REGION 1..120: note = VH domain of 9412 IgG histidine scanning variant #5
EVQLVESGGGLVQPGGSLRLSCTASGFSLHTYAMSWVRQAPGKGLEWVGMIYINDNTYYASWAKGRFTISRDTSKNTVYLQMNSLRAEDTAVYFCARDIGISYYIGVSLWGQGTLVTVSS

SEQ ID NO: 555 - protein, 120 aa, synthetic construct
  REGION 1..120: note = VH domain of 9412 IgG histidine scanning variant #6
EVQLVESGGGLVQPGGSLRLSCTASGFSLSHYAMSWVRQAPGKGLEWVGMIYINDNTYYASWAKGRFTISRDTSKNTVYLQMNSLRAEDTAVYFCARDIGISYYIGVSLWGQGTLVTVSS

SEQ ID NO: 556 - protein, 120 aa, synthetic construct
  REGION 1..120: note = VH domain of 9412 IgG histidine scanning variant #7
EVQLVESGGGLVQPGGSLRLSCTASGFSLSTHAMSWVRQAPGKGLEWVGMIYINDNTYYASWAKGRFTISRDTSKNTVYLQMNSLRAEDTAVYFCARDIGISYYIGVSLWGQGTLVTVSS

SEQ ID NO: 557 - protein, 120 aa, synthetic construct
  REGION 1..120: note = VH domain of 9412 IgG histidine scanning variant #8
EVQLVESGGGLVQPGGSLRLSCTASGFSLSTYHMSWVRQAPGKGLEWVGMIYINDNTYYASWAKGRFTISRDTSKNTVYLQMNSLRAEDTAVYFCARDIGISYYIGVSLWGQGTLVTVSS

SEQ ID NO: 558 - protein, 120 aa, synthetic construct
  REGION 1..120: note = VH domain of 9412 IgG histidine scanning variant #9
EVQLVESGGGLVQPGGSLRLSCTASGFSLSTYAHSWVRQAPGKGLEWVGMIYINDNTYYASWAKGRFTISRDTSKNTVYLQMNSLRAEDTAVYFCARDIGISYYIGVSLWGQGTLVTVSS

SEQ ID NO: 559 - protein, 120 aa, synthetic construct
  REGION 1..120: note = VH domain of 9412 IgG histidine scanning variant #10
EVQLVESGGGLVQPGGSLRLSCTASGFSLSTYAMHWVRQAPGKGLEWVGMIYINDNTYYASWAKGRFTISRDTSKNTVYLQMNSLRAEDTAVYFCARDIGISYYIGVSLWGQGTLVTVSS

SEQ ID NO: 560 - protein, 120 aa, synthetic construct
  REGION 1..120: note = VH domain of 9412 IgG histidine scanning variant #11
EVQLVESGGGLVQPGGSLRLSCTASGFSLSTYAMSWVRQAPGKGLEWVGHIYINDNTYYASWAKGRFTISRDTSKNTVYLQMNSLRAEDTAVYFCARDIGISYYIGVSLWGQGTLVTVSS

SEQ ID NO: 561 - protein, 120 aa, synthetic construct
  REGION 1..120: note = VH domain of 9412 IgG histidine scanning variant #12
EVQLVESGGGLVQPGGSLRLSCTASGFSLSTYAMSWVRQAPGKGLEWVGMHYINDNTYYASWAKGRFTISRDTSKNTVYLQMNSLRAEDTAVYFCARDIGISYYIGVSLWGQGTLVTVSS

SEQ ID NO: 562 - protein, 120 aa, synthetic construct
  REGION 1..120: note = VH domain of 9412 IgG histidine scanning variant #13
EVQLVESGGGLVQPGGSLRLSCTASGFSLSTYAMSWVRQAPGKGLEWVGMIHINDNTYYASWAKGRFTISRDTSKNTVYLQMNSLRAEDTAVYFCARDIGISYYIGVSLWGQGTLVTVSS

SEQ ID NO: 563 - protein, 120 aa, synthetic construct
  REGION 1..120: note = VH domain of 9412 IgG histidine scanning variant #14
EVQLVESGGGLVQPGGSLRLSCTASGFSLSTYAMSWVRQAPGKGLEWVGMIYHNDNTYYASWAKGRFTISRDTSKNTVYLQMNSLRAEDTAVYFCARDIGISYYIGVSLWGQGTLVTVSS

SEQ ID NO: 564 - protein, 120 aa, synthetic construct
  REGION 1..120: note = VH domain of 9412 IgG histidine scanning variant #15
EVQLVESGGGLVQPGGSLRLSCTASGFSLSTYAMSWVRQAPGKGLEWVGMIYIHDNTYYASWAKGRFTISRDTSKNTVYLQMNSLRAEDTAVYFCARDIGISYYIGVSLWGQGTLVTVSS

SEQ ID NO: 565 - protein, 120 aa, synthetic construct
  REGION 1..120: note = VH domain of 9412 IgG histidine scanning variant #16
EVQLVESGGGLVQPGGSLRLSCTASGFSLSTYAMSWVRQAPGKGLEWVGMIYINHNTYYASWAKGRFTISRDTSKNTVYLQMNSLRAEDTAVYFCARDIGISYYIGVSLWGQGTLVTVSS

SEQ ID NO: 566 - protein, 120 aa, synthetic construct
  REGION 1..120: note = VH domain of 9412 IgG histidine scanning variant #17
EVQLVESGGGLVQPGGSLRLSCTASGFSLSTYAMSWVRQAPGKGLEWVGMIYINDHTYYASWAKGRFTISRDTSKNTVYLQMNSLRAEDTAVYFCARDIGISYYIGVSLWGQGTLVTVSS

SEQ ID NO: 567 - protein, 120 aa, synthetic construct
  REGION 1..120: note = VH domain of 9412 IgG histidine scanning variant #18
EVQLVESGGGLVQPGGSLRLSCTASGFSLSTYAMSWVRQAPGKGLEWVGMIYINDNHYYASWAKGRFTISRDTSKNTVYLQMNSLRAEDTAVYFCARDIGISYYIGVSLWGQGTLVTVSS

SEQ ID NO: 568 - protein, 120 aa, synthetic construct
  REGION 1..120: note = VH domain of 9412 IgG histidine scanning variant #19
EVQLVESGGGLVQPGGSLRLSCTASGFSLSTYAMSWVRQAPGKGLEWVGMIYINDNTHYASWAKGRFTISRDTSKNTVYLQMNSLRAEDTAVYFCARDIGISYYIGVSLWGQGTLVTVSS

SEQ ID NO: 569 - protein, 120 aa, synthetic construct
  REGION 1..120: note = VH domain of 9412 IgG histidine scanning variant #20
EVQLVESGGGLVQPGGSLRLSCTASGFSLSTYAMSWVRQAPGKGLEWVGMIYINDNTYHASWAKGRFTISRDTSKNTVYLQMNSLRAEDTAVYFCARDIGISYYIGVSLWGQGTLVTVSS

SEQ ID NO: 570 - protein, 120 aa, synthetic construct
  REGION 1..120: note = VH domain of 9412 IgG histidine scanning variant #21
EVQLVESGGGLVQPGGSLRLSCTASGFSLSTYAMSWVRQAPGKGLEWVGMIYINDNTYYHSWAKGRFTISRDTSKNTVYLQMNSLRAEDTAVYFCARDIGISYYIGVSLWGQGTLVTVSS

SEQ ID NO: 571 - protein, 120 aa, synthetic construct
  REGION 1..120: note = VH domain of 9412 IgG histidine scanning variant #22
EVQLVESGGGLVQPGGSLRLSCTASGFSLSTYAMSWVRQAPGKGLEWVGMIYINDNTYYAHWAKGRFTISRDTSKNTVYLQMNSLRAEDTAVYFCARDIGISYYIGVSLWGQGTLVTVSS

SEQ ID NO: 572 - protein, 120 aa, synthetic construct
  REGION 1..120: note = VH domain of 9412 IgG histidine scanning variant #23
EVQLVESGGGLVQPGGSLRLSCTASGFSLSTYAMSWVRQAPGKGLEWVGMIYINDNTYYASHAKGRFTISRDTSKNTVYLQMNSLRAEDTAVYFCARDIGISYYIGVSLWGQGTLVTVSS

SEQ ID NO: 573 - protein, 120 aa, synthetic construct
  REGION 1..120: note = VH domain of 9412 IgG histidine scanning variant #24
EVQLVESGGGLVQPGGSLRLSCTASGFSLSTYAMSWVRQAPGKGLEWVGMIYINDNTYYASWHKGRFTISRDTSKNTVYLQMNSLRAEDTAVYFCARDIGISYYIGVSLWGQGTLVTVSS

SEQ ID NO: 574 - protein, 120 aa, synthetic construct
  REGION 1..120: note = VH domain of 9412 IgG histidine scanning variant #25
EVQLVESGGGLVQPGGSLRLSCTASGFSLSTYAMSWVRQAPGKGLEWVGMIYINDNTYYASWAHGRFTISRDTSKNTVYLQMNSLRAEDTAVYFCARDIGISYYIGVSLWGQGTLVTVSS

SEQ ID NO: 575 - protein, 120 aa, synthetic construct
  REGION 1..120: note = VH domain of 9412 IgG histidine scanning variant #26
EVQLVESGGGLVQPGGSLRLSCTASGFSLSTYAMSWVRQAPGKGLEWVGMIYINDNTYYASWAKHRFTISRDTSKNTVYLQMNSLRAEDTAVYFCARDIGISYYIGVSLWGQGTLVTVSS

SEQ ID NO: 576 - protein, 120 aa, synthetic construct
  REGION 1..120: note = VH domain of 9412 IgG histidine scanning variant #27
EVQLVESGGGLVQPGGSLRLSCTASGFSLSTYAMSWVRQAPGKGLEWVGMIYINDNTYYASWAKGRFTISRDTSKNTVYLQMNSLRAEDTAVYFCHRDIGISYYIGVSLWGQGTLVTVSS

SEQ ID NO: 577 - protein, 120 aa, synthetic construct
  REGION 1..120: note = VH domain of 9412 IgG histidine scanning variant #28
EVQLVESGGGLVQPGGSLRLSCTASGFSLSTYAMSWVRQAPGKGLEWVGMIYINDNTYYASWAKGRFTISRDTSKNTVYLQMNSLRAEDTAVYFCAHDIGISYYIGVSLWGQGTLVTVSS

SEQ ID NO: 578 - protein, 120 aa, synthetic construct
  REGION 1..120: note = VH domain of 9412 IgG histidine scanning variant #29
EVQLVESGGGLVQPGGSLRLSCTASGFSLSTYAMSWVRQAPGKGLEWVGMIYINDNTYYASWAKGRFTISRDTSKNTVYLQMNSLRAEDTAVYFCARHIGISYYIGVSLWGQGTLVTVSS

SEQ ID NO: 579 - protein, 120 aa, synthetic construct
  REGION 1..120: note = VH domain of 9412 IgG histidine scanning variant #30
EVQLVESGGGLVQPGGSLRLSCTASGFSLSTYAMSWVRQAPGKGLEWVGMIYINDNTYYASWAKGRFTISRDTSKNTVYLQMNSLRAEDTAVYFCARDHGISYYIGVSLWGQGTLVTVSS

SEQ ID NO: 580 - protein, 120 aa, synthetic construct
  REGION 1..120: note = VH domain of 9412 IgG histidine scanning variant #31
EVQLVESGGGLVQPGGSLRLSCTASGFSLSTYAMSWVRQAPGKGLEWVGMIYINDNTYYASWAKGRFTISRDTSKNTVYLQMNSLRAEDTAVYFCARDIHISYYIGVSLWGQGTLVTVSS

SEQ ID NO: 581 - protein, 120 aa, synthetic construct
  REGION 1..120: note = VH domain of 9412 IgG histidine scanning variant #32
EVQLVESGGGLVQPGGSLRLSCTASGFSLSTYAMSWVRQAPGKGLEWVGMIYINDNTYYASWAKGRFTISRDTSKNTVYLQMNSLRAEDTAVYFCARDIGHSYYIGVSLWGQGTLVTVSS

SEQ ID NO: 582 - protein, 120 aa, synthetic construct
  REGION 1..120: note = VH domain of 9412 IgG histidine scanning variant #33
EVQLVESGGGLVQPGGSLRLSCTASGFSLSTYAMSWVRQAPGKGLEWVGMIYINDNTYYASWAKGRFTISRDTSKNTVYLQMNSLRAEDTAVYFCARDIGIHYYIGVSLWGQGTLVTVSS

SEQ ID NO: 583 - protein, 120 aa, synthetic construct
  REGION 1..120: note = VH domain of 9412 IgG histidine scanning variant #34
EVQLVESGGGLVQPGGSLRLSCTASGFSLSTYAMSWVRQAPGKGLEWVGMIYINDNTYYASWAKGRFTISRDTSKNTVYLQMNSLRAEDTAVYFCARDIGISHYIGVSLWGQGTLVTVSS

SEQ ID NO: 584 - protein, 120 aa, synthetic construct
  REGION 1..120: note = VH domain of 9412 IgG histidine scanning variant #35
EVQLVESGGGLVQPGGSLRLSCTASGFSLSTYAMSWVRQAPGKGLEWVGMIYINDNTYYASWAKGRFTISRDTSKNTVYLQMNSLRAEDTAVYFCARDIGISYHIGVSLWGQGTLVTVSS

SEQ ID NO: 585 - protein, 120 aa, synthetic construct
  REGION 1..120: note = VH domain of 9412 IgG histidine scanning variant #36
EVQLVESGGGLVQPGGSLRLSCTASGFSLSTYAMSWVRQAPGKGLEWVGMIYINDNTYYASWAKGRFTISRDTSKNTVYLQMNSLRAEDTAVYFCARDIGISYYHGVSLWGQGTLVTVSS

SEQ ID NO: 586 - protein, 120 aa, synthetic construct
  REGION 1..120: note = VH domain of 9412 IgG histidine scanning variant #37
EVQLVESGGGLVQPGGSLRLSCTASGFSLSTYAMSWVRQAPGKGLEWVGMIYINDNTYYASWAKGRFTISRDTSKNTVYLQMNSLRAEDTAVYFCARDIGISYYIHVSLWGQGTLVTVSS

SEQ ID NO: 587 - protein, 120 aa, synthetic construct
  REGION 1..120: note = VH domain of 9412 IgG histidine scanning variant #38
EVQLVESGGGLVQPGGSLRLSCTASGFSLSTYAMSWVRQAPGKGLEWVGMIYINDNTYYASWAKGRFTISRDTSKNTVYLQMNSLRAEDTAVYFCARDIGISYYIGHSLWGQGTLVTVSS

SEQ ID NO: 588 - protein, 120 aa, synthetic construct
  REGION 1..120: note = VH domain of 9412 IgG histidine scanning variant #39
EVQLVESGGGLVQPGGSLRLSCTASGFSLSTYAMSWVRQAPGKGLEWVGMIYINDNTYYASWAKGRFTISRDTSKNTVYLQMNSLRAEDTAVYFCARDIGISYYIGVHLWGQGTLVTVSS

SEQ ID NO: 589 - protein, 120 aa, synthetic construct
  REGION 1..120: note = VH domain of 9412 IgG histidine scanning variant #40
EVQLVESGGGLVQPGGSLRLSCTASGFSLSTYAMSWVRQAPGKGLEWVGMIYINDNTYYASWAKGRFTISRDTSKNTVYLQMNSLRAEDTAVYFCARDIGISYYIGVSHWGQGTLVTVSS

SEQ ID NO: 590 - protein, 120 aa, synthetic construct
  REGION 1..120: note = VH domain of 9412 IgG histidine scanning variant #41
EVQLVESGGGLVQPGGSLRLSCTASGFSLSTYAMSWVRQAPGKGLEWVGMIYINDNTYYASWAKGRFTISRDTSKNTVYLQMNSLRAEDTAVYYCARDIGISYYIGVSLWGQGTLVTVSS

SEQ ID NO: 591 - protein, 120 aa, synthetic construct
  REGION 1..120: note = VH domain of 9412 IgG histidine scanning variant #42
EVQLVESGGGLVQPGGSLRLSCTASGFSLSTYAMSWVRQAPGKGLEWVGMIYINANTYYASWAKGRFTISRDTSKNTVYLQMNSLRAEDTAVYFCARDIGISYYIGVSLWGQGTLVTVSS

SEQ ID NO: 592 - protein, 120 aa, synthetic construct
  REGION 1..120: note = VH domain of 9412 IgG histidine scanning variant #43
EVQLVESGGGLVQPGGSLRLSCTASGFSLSTYAMSWVRQAPGKGLEWVGMIYINDNTYYASWAKGRFTISRDTSKNTVYLQMNSLRAEDTAVYFCARAIGISYYIGVSLWGQGTLVTVSS

SEQ ID NO: 593 - protein, 111 aa, synthetic construct
  REGION 1..111: note = VL domain of 9412 IgG histidine scanning variant #1
EIVMTQSPSTLSASVGDRVIITCHASQSIGTRLSWYQQKPGKAPKLLIYGASKLATGVPSRFSGSGSGAEFTLTISSLQPDDFATYYCLGEYSYTTTDGIAFGQGTKLTVL

SEQ ID NO: 594 - protein, 111 aa, synthetic construct
  REGION 1..111: note = VL domain of 9412 IgG histidine scanning variant #2
EIVMTQSPSTLSASVGDRVIITCQHSQSIGTRLSWYQQKPGKAPKLLIYGASKLATGVPSRFSGSGSGAEFTLTISSLQPDDFATYYCLGEYSYTTTDGIAFGQGTKLTVL

SEQ ID NO: 595 - protein, 111 aa, synthetic construct
  REGION 1..111: note = VL domain of 9412 IgG histidine scanning variant #3
EIVMTQSPSTLSASVGDRVIITCQAHQSIGTRLSWYQQKPGKAPKLLIYGASKLATGVPSRFSGSGSGAEFTLTISSLQPDDFATYYCLGEYSYTTTDGIAFGQGTKLTVL

SEQ ID NO: 596 - protein, 111 aa, synthetic construct
  REGION 1..111: note = VL domain of 9412 IgG histidine scanning variant #4
EIVMTQSPSTLSASVGDRVIITCQASHSIGTRLSWYQQKPGKAPKLLIYGASKLATGVPSRFSGSGSGAEFTLTISSLQPDDFATYYCLGEYSYTTTDGIAFGQGTKLTVL

SEQ ID NO: 597 - protein, 111 aa, synthetic construct
  REGION 1..111: note = VL domain of 9412 IgG histidine scanning variant #5
EIVMTQSPSTLSASVGDRVIITCQASQHIGTRLSWYQQKPGKAPKLLIYGASKLATGVPSRFSGSGSGAEFTLTISSLQPDDFATYYCLGEYSYTTTDGIAFGQGTKLTVL

SEQ ID NO: 598 - protein, 111 aa, synthetic construct
  REGION 1..111: note = VL domain of 9412 IgG histidine scanning variant #6
EIVMTQSPSTLSASVGDRVIITCQASQSHGTRLSWYQQKPGKAPKLLIYGASKLATGVPSRFSGSGSGAEFTLTISSLQPDDFATYYCLGEYSYTTTDGIAFGQGTKLTVL

SEQ ID NO: 599 - protein, 111 aa, synthetic construct
  REGION 1..111: note = VL domain of 9412 IgG histidine scanning variant #7
EIVMTQSPSTLSASVGDRVIITCQASQSIHTRLSWYQQKPGKAPKLLIYGASKLATGVPSRFSGSGSGAEFTLTISSLQPDDFATYYCLGEYSYTTTDGIAFGQGTKLTVL

SEQ ID NO: 600 - protein, 111 aa, synthetic construct
  REGION 1..111: note = VL domain of 9412 IgG histidine scanning variant #8
EIVMTQSPSTLSASVGDRVIITCQASQSIGHRLSWYQQKPGKAPKLLIYGASKLATGVPSRFSGSGSGAEFTLTISSLQPDDFATYYCLGEYSYTTTDGIAFGQGTKLTVL

SEQ ID NO: 601 - protein, 111 aa, synthetic construct
  REGION 1..111: note = VL domain of 9412 IgG histidine scanning variant #9
EIVMTQSPSTLSASVGDRVIITCQASQSIGTHLSWYQQKPGKAPKLLIYGASKLATGVPSRFSGSGSGAEFTLTISSLQPDDFATYYCLGEYSYTTTDGIAFGQGTKLTVL

SEQ ID NO: 602 - protein, 111 aa, synthetic construct
  REGION 1..111: note = VL domain of 9412 IgG histidine scanning variant #10
EIVMTQSPSTLSASVGDRVIITCQASQSIGTRHSWYQQKPGKAPKLLIYGASKLATGVPSRFSGSGSGAEFTLTISSLQPDDFATYYCLGEYSYTTTDGIAFGQGTKLTVL

SEQ ID NO: 603 - protein, 111 aa, synthetic construct
  REGION 1..111: note = VL domain of 9412 IgG histidine scanning variant #11
EIVMTQSPSTLSASVGDRVIITCQASQSIGTRLHWYQQKPGKAPKLLIYGASKLATGVPSRFSGSGSGAEFTLTISSLQPDDFATYYCLGEYSYTTTDGIAFGQGTKLTVL

SEQ ID NO: 604 - protein, 111 aa, synthetic construct
  REGION 1..111: note = VL domain of 9412 IgG histidine scanning variant #12
EIVMTQSPSTLSASVGDRVIITCQASQSIGTRLSWYQQKPGKAPKLLIYHASKLATGVPSRFSGSGSGAEFTLTISSLQPDDFATYYCLGEYSYTTTDGIAFGQGTKLTVL

SEQ ID NO: 605 - protein, 111 aa, synthetic construct
  REGION 1..111: note = VL domain of 9412 IgG histidine scanning variant #13
EIVMTQSPSTLSASVGDRVIITCQASQSIGTRLSWYQQKPGKAPKLLIYGHSKLATGVPSRFSGSGSGAEFTLTISSLQPDDFATYYCLGEYSYTTTDGIAFGQGTKLTVL

SEQ ID NO: 606 - protein, 111 aa, synthetic construct
  REGION 1..111: note = VL domain of 9412 IgG histidine scanning variant #14
EIVMTQSPSTLSASVGDRVIITCQASQSIGTRLSWYQQKPGKAPKLLIYGAHKLATGVPSRFSGSGSGAEFTLTISSLQPDDFATYYCLGEYSYTTTDGIAFGQGTKLTVL

SEQ ID NO: 607 - protein, 111 aa, synthetic construct
  REGION 1..111: note = VL domain of 9412 IgG histidine scanning variant #15
EIVMTQSPSTLSASVGDRVIITCQASQSIGTRLSWYQQKPGKAPKLLIYGASHLATGVPSRFSGSGSGAEFTLTISSLQPDDFATYYCLGEYSYTTTDGIAFGQGTKLTVL

SEQ ID NO: 608 - protein, 111 aa, synthetic construct
  REGION 1..111: note = VL domain of 9412 IgG histidine scanning variant #16
EIVMTQSPSTLSASVGDRVIITCQASQSIGTRLSWYQQKPGKAPKLLIYGASKHATGVPSRFSGSGSGAEFTLTISSLQPDDFATYYCLGEYSYTTTDGIAFGQGTKLTVL

SEQ ID NO: 609 - protein, 111 aa, synthetic construct
  REGION 1..111: note = VL domain of 9412 IgG histidine scanning variant #17
EIVMTQSPSTLSASVGDRVIITCQASQSIGTRLSWYQQKPGKAPKLLIYGASKLHTGVPSRFSGSGSGAEFTLTISSLQPDDFATYYCLGEYSYTTTDGIAFGQGTKLTVL

SEQ ID NO: 610 - protein, 111 aa, synthetic construct
  REGION 1..111: note = VL domain of 9412 IgG histidine scanning variant #18
EIVMTQSPSTLSASVGDRVIITCQASQSIGTRLSWYQQKPGKAPKLLIYGASKLAHGVPSRFSGSGSGAEFTLTISSLQPDDFATYYCLGEYSYTTTDGIAFGQGTKLTVL

SEQ ID NO: 611 - protein, 111 aa, synthetic construct
  REGION 1..111: note = VL domain of 9412 IgG histidine scanning variant #19
EIVMTQSPSTLSASVGDRVIITCQASQSIGTRLSWYQQKPGKAPKLLIYGASKLATGVPSRFSGSGSGAEFTLTISSLQPDDFATYYCHGEYSYTTTDGIAFGQGTKLTVL

SEQ ID NO: 612 - protein, 111 aa, synthetic construct
  REGION 1..111: note = VL domain of 9412 IgG histidine scanning variant #20
EIVMTQSPSTLSASVGDRVIITCQASQSIGTRLSWYQQKPGKAPKLLIYGASKLATGVPSRFSGSGSGAEFTLTISSLQPDDFATYYCLHEYSYTTTDGIAFGQGTKLTVL

SEQ ID NO: 613 - protein, 111 aa, synthetic construct
  REGION 1..111: note = VL domain of 9412 IgG histidine scanning variant #21
EIVMTQSPSTLSASVGDRVIITCQASQSIGTRLSWYQQKPGKAPKLLIYGASKLATGVPSRFSGSGSGAEFTLTISSLQPDDFATYYCLGHYSYTTTDGIAFGQGTKLTVL

SEQ ID NO: 614 - protein, 111 aa, synthetic construct
  REGION 1..111: note = VL domain of 9412 IgG histidine scanning variant #22
EIVMTQSPSTLSASVGDRVIITCQASQSIGTRLSWYQQKPGKAPKLLIYGASKLATGVPSRFSGSGSGAEFTLTISSLQPDDFATYYCLGEHSYTTTDGIAFGQGTKLTVL

SEQ ID NO: 615 - protein, 111 aa, synthetic construct
  REGION 1..111: note = VL domain of 9412 IgG histidine scanning variant #23
EIVMTQSPSTLSASVGDRVIITCQASQSIGTRLSWYQQKPGKAPKLLIYGASKLATGVPSRFSGSGSGAEFTLTISSLQPDDFATYYCLGEYHYTTTDGIAFGQGTKLTVL

SEQ ID NO: 616 - protein, 111 aa, synthetic construct
  REGION 1..111: note = VL domain of 9412 IgG histidine scanning variant #24
EIVMTQSPSTLSASVGDRVIITCQASQSIGTRLSWYQQKPGKAPKLLIYGASKLATGVPSRFSGSGSGAEFTLTISSLQPDDFATYYCLGEYSHTTTDGIAFGQGTKLTVL

SEQ ID NO: 617 - protein, 111 aa, synthetic construct
  REGION 1..111: note = VL domain of 9412 IgG histidine scanning variant #25
EIVMTQSPSTLSASVGDRVIITCQASQSIGTRLSWYQQKPGKAPKLLIYGASKLATGVPSRFSGSGSGAEFTLTISSLQPDDFATYYCLGEYSYHTTDGIAFGQGTKLTVL

SEQ ID NO: 618 - protein, 111 aa, synthetic construct
  REGION 1..111: note = VL domain of 9412 IgG histidine scanning variant #26
EIVMTQSPSTLSASVGDRVIITCQASQSIGTRLSWYQQKPGKAPKLLIYGASKLATGVPSRFSGSGSGAEFTLTISSLQPDDFATYYCLGEYSYTHTDGIAFGQGTKLTVL

SEQ ID NO: 619 - protein, 111 aa, synthetic construct
  REGION 1..111: note = VL domain of 9412 IgG histidine scanning variant #27
EIVMTQSPSTLSASVGDRVIITCQASQSIGTRLSWYQQKPGKAPKLLIYGASKLATGVPSRFSGSGSGAEFTLTISSLQPDDFATYYCLGEYSYTTHDGIAFGQGTKLTVL

SEQ ID NO: 620 - protein, 111 aa, synthetic construct
  REGION 1..111: note = VL domain of 9412 IgG histidine scanning variant #28
EIVMTQSPSTLSASVGDRVIITCQASQSIGTRLSWYQQKPGKAPKLLIYGASKLATGVPSRFSGSGSGAEFTLTISSLQPDDFATYYCLGEYSYTTTHGIAFGQGTKLTVL

SEQ ID NO: 621 - protein, 111 aa, synthetic construct
  REGION 1..111: note = VL domain of 9412 IgG histidine scanning variant #29
EIVMTQSPSTLSASVGDRVIITCQASQSIGTRLSWYQQKPGKAPKLLIYGASKLATGVPSRFSGSGSGAEFTLTISSLQPDDFATYYCLGEYSYTTTDHIAFGQGTKLTVL

SEQ ID NO: 622 - protein, 111 aa, synthetic construct
  REGION 1..111: note = VL domain of 9412 IgG histidine scanning variant #30
EIVMTQSPSTLSASVGDRVIITCQASQSIGTRLSWYQQKPGKAPKLLIYGASKLATGVPSRFSGSGSGAEFTLTISSLQPDDFATYYCLGEYSYTTTDGHAFGQGTKLTVL

SEQ ID NO: 623 - protein, 111 aa, synthetic construct
  REGION 1..111: note = VL domain of 9412 IgG histidine scanning variant #31
EIVMTQSPSTLSASVGDRVIITCQASQSIGTRLSWYQQKPGKAPKLLIYGASKLATGVPSRFSGSGSGAEFTLTISSLQPDDFATYYCLGEYSYTTTDGIHFGQGTKLTVL

SEQ ID NO: 624 - protein, 111 aa, synthetic construct
  REGION 1..111: note = VL domain of 9412 IgG histidine scanning variant #32
EIVMTQSPSTLSASVGDRVIITCQASQSIGTRLSWYQQKPGKAPKLLIYGASKLATGVPSRFSGSGSGAEFTLTISSLQPDDFATYYCLGEYSYTTTDGIAFGQGTKLEIK

SEQ ID NO: 625 - protein, 111 aa, synthetic construct
  REGION 1..111: note = VL domain of 9412 IgG histidine scanning variant #33
EIVMTQSPSTLSASVGDRVIITCQASQSIGTRLSWYQQKPGKAPKLLIYGASKLATGVPSRFSGSGSGAEFTLTISSLQPDDFATYYCLGEYSYTTTAGIAFGQGTKLTVL

SEQ ID NO: 626 - protein, 111 aa, synthetic construct
  REGION 1..111: note = VL domain of 9412 IgG histidine scanning variant #34
EIVMTQSPSTLSASVGDRVIITCQASQSIGTRLSWYQQKPGKAPKLLIYGASKLATGVPSRFSGSGSGAEFTLTISSLQPDDFATYYCLGEYSYTTTDAIAFGQGTKLTVL

SEQ ID NO: 627 - protein, 111 aa, synthetic construct
  REGION 1..111: note = VL domain of 9412 IgG histidine scanning variant #35
EIVMTQSPSTLSASVGDRVIITCQASQSIGTRLSWYQQKPGKAPKLLIYGASKLATGVPSRFSGSGSGAEFTLTISSLQPDDFATYYCLGAYSYTTTDGIAFGQGTKLTVL

SEQ ID NO: 628 - protein, 117 aa, synthetic construct
  REGION 1..117: note = VH domain of 9460 IgG
EVQLVESGGGLVQPGGSLRLSCTASGIDLSSYVMGWVRQAPGKGLEWVGYIYGGGGTYYATWARGRFTISRDTSKNTVYLQMNSLRAEDTAVYFCARAAADFYFNLWGQGTLVTVSS

SEQ ID NO: 629 - protein, 110 aa, synthetic construct
  REGION 1..110: note = VL domain of 9460 IgG
EIVMTQSPSTLSASVGDRVIITCQASQSISSYLAWYQQKPGKAPKLLIYDISDLASGVPSRFSGSGSGAEFTLTISSLQPDDFATYYCQSYYWISNSGAAFGQGTKLTVL

SEQ ID NO: 630 - protein, 10 aa, synthetic construct
  REGION 1..10: note = Heavy Chain CDR1 of 9460 IgG
GIDLSSYVMG

SEQ ID NO: 631 - protein, 16 aa, synthetic construct
  REGION 1..16: note = Heavy Chain CDR2 of 9460 IgG
YIYGGGGTYYATWARG

SEQ ID NO: 632 - protein, 11 aa, synthetic construct
  REGION 1..11: note = Heavy Chain CDR3 of 9460 IgG
ARAAADFYFNL

SEQ ID NO: 633 - protein, 11 aa, synthetic construct
  REGION 1..11: note = Light Chain CDR1 of 9460 IgG
QASQSISSYLA

SEQ ID NO: 634 - protein, 7 aa, synthetic construct
  REGION 1..7: note = Light Chain CDR2 of 9460 IgG
DISDLAS

SEQ ID NO: 635 - protein, 12 aa, synthetic construct
  REGION 1..12: note = Light Chain CDR3 of 9460 IgG
QSYYWISNSGAA

SEQ ID NO: 636 - protein, 117 aa, synthetic construct
  REGION 1..117: note = VH domain of 9460 IgG histidine scanning variant #1
EVQLVESGGGLVQPGGSLRLSCTASHIDLSSYVMGWVRQAPGKGLEWVGYIYGGGGTYYATWARGRFTISRDTSKNTVYLQMNSLRAEDTAVYFCARAAADFYFNLWGQGTLVTVSS

SEQ ID NO: 637 - protein, 117 aa, synthetic construct
  REGION 1..117: note = VH domain of 9460 IgG histidine scanning variant #2
EVQLVESGGGLVQPGGSLRLSCTASGHDLSSYVMGWVRQAPGKGLEWVGYIYGGGGTYYATWARGRFTISRDTSKNTVYLQMNSLRAEDTAVYFCARAAADFYFNLWGQGTLVTVSS

SEQ ID NO: 638 - protein, 117 aa, synthetic construct
  REGION 1..117: note = VH domain of 9460 IgG histidine scanning variant #3
EVQLVESGGGLVQPGGSLRLSCTASGIHLSSYVMGWVRQAPGKGLEWVGYIYGGGGTYYATWARGRFTISRDTSKNTVYLQMNSLRAEDTAVYFCARAAADFYFNLWGQGTLVTVSS

SEQ ID NO: 639 - protein, 117 aa, synthetic construct
  REGION 1..117: note = VH domain of 9460 IgG histidine scanning variant #4
EVQLVESGGGLVQPGGSLRLSCTASGIDHSSYVMGWVRQAPGKGLEWVGYIYGGGGTYYATWARGRFTISRDTSKNTVYLQMNSLRAEDTAVYFCARAAADFYFNLWGQGTLVTVSS

SEQ ID NO: 640 - protein, 117 aa, synthetic construct
  REGION 1..117: note = VH domain of 9460 IgG histidine scanning variant #5
EVQLVESGGGLVQPGGSLRLSCTASGIDLHSYVMGWVRQAPGKGLEWVGYIYGGGGTYYATWARGRFTISRDTSKNTVYLQMNSLRAEDTAVYFCARAAADFYFNLWGQGTLVTVSS

SEQ ID NO: 641 - protein, 117 aa, synthetic construct
  REGION 1..117: note = VH domain of 9460 IgG histidine scanning variant #6
EVQLVESGGGLVQPGGSLRLSCTASGIDLSHYVMGWVRQAPGKGLEWVGYIYGGGGTYYATWARGRFTISRDTSKNTVYLQMNSLRAEDTAVYFCARAAADFYFNLWGQGTLVTVSS

SEQ ID NO: 642 - protein, 117 aa, synthetic construct
  REGION 1..117: note = VH domain of 9460 IgG histidine scanning variant #7
EVQLVESGGGLVQPGGSLRLSCTASGIDLSSHVMGWVRQAPGKGLEWVGYIYGGGGTYYATWARGRFTISRDTSKNTVYLQMNSLRAEDTAVYFCARAAADFYFNLWGQGTLVTVSS

SEQ ID NO: 643 - protein, 117 aa, synthetic construct
  REGION 1..117: note = VH domain of 9460 IgG histidine scanning variant #8
EVQLVESGGGLVQPGGSLRLSCTASGIDLSSYHMGWVRQAPGKGLEWVGYIYGGGGTYYATWARGRFTISRDTSKNTVYLQMNSLRAEDTAVYFCARAAADFYFNLWGQGTLVTVSS

SEQ ID NO: 644 - protein, 117 aa, synthetic construct
  REGION 1..117: note = VH domain of 9460 IgG histidine scanning variant #9
EVQLVESGGGLVQPGGSLRLSCTASGIDLSSYVHGWVRQAPGKGLEWVGYIYGGGGTYYATWARGRFTISRDTSKNTVYLQMNSLRAEDTAVYFCARAAADFYFNLWGQGTLVTVSS

SEQ ID NO: 645 - protein, 117 aa, synthetic construct
  REGION 1..117: note = VH domain of 9460 IgG histidine scanning variant #10
EVQLVESGGGLVQPGGSLRLSCTASGIDLSSYVMHWVRQAPGKGLEWVGYIYGGGGTYYATWARGRFTISRDTSKNTVYLQMNSLRAEDTAVYFCARAAADFYFNLWGQGTLVTVSS

SEQ ID NO: 646 - protein, 117 aa, synthetic construct
  REGION 1..117: note = VH domain of 9460 IgG histidine scanning variant #11
EVQLVESGGGLVQPGGSLRLSCTASGIDLSSYVMGWVRQAPGKGLEWVGHIYGGGGTYYATWARGRFTISRDTSKNTVYLQMNSLRAEDTAVYFCARAAADFYFNLWGQGTLVTVSS

SEQ ID NO: 647 - protein, 117 aa, synthetic construct
  REGION 1..117: note = VH domain of 9460 IgG histidine scanning variant #12
EVQLVESGGGLVQPGGSLRLSCTASGIDLSSYVMGWVRQAPGKGLEWVGYHYGGGGTYYATWARGRFTISRDTSKNTVYLQMNSLRAEDTAVYFCARAAADFYFNLWGQGTLVTVSS

SEQ ID NO: 648 - protein, 117 aa, synthetic construct
  REGION 1..117: note = VH domain of 9460 IgG histidine scanning variant #13
EVQLVESGGGLVQPGGSLRLSCTASGIDLSSYVMGWVRQAPGKGLEWVGYIHGGGGTYYATWARGRFTISRDTSKNTVYLQMNSLRAEDTAVYFCARAAADFYFNLWGQGTLVTVSS

SEQ ID NO: 649 - protein, 117 aa, synthetic construct
  REGION 1..117: note = VH domain of 9460 IgG histidine scanning variant #14
EVQLVESGGGLVQPGGSLRLSCTASGIDLSSYVMGWVRQAPGKGLEWVGYIYHGGGTYYATWARGRFTISRDTSKNTVYLQMNSLRAEDTAVYFCARAAADFYFNLWGQGTLVTVSS

SEQ ID NO: 650 - protein, 117 aa, synthetic construct
  REGION 1..117: note = VH domain of 9460 IgG histidine scanning variant #15
EVQLVESGGGLVQPGGSLRLSCTASGIDLSSYVMGWVRQAPGKGLEWVGYIYGHGGTYYATWARGRFTISRDTSKNTVYLQMNSLRAEDTAVYFCARAAADFYFNLWGQGTLVTVSS

SEQ ID NO: 651 - protein, 117 aa, synthetic construct
  REGION 1..117: note = VH domain of 9460 IgG histidine scanning variant #16
EVQLVESGGGLVQPGGSLRLSCTASGIDLSSYVMGWVRQAPGKGLEWVGYIYGGHGTYYATWARGRFTISRDTSKNTVYLQMNSLRAEDTAVYFCARAAADFYFNLWGQGTLVTVSS

SEQ ID NO: 652 - protein, 117 aa, synthetic construct
  REGION 1..117: note = VH domain of 9460 IgG histidine scanning variant #17
EVQLVESGGGLVQPGGSLRLSCTASGIDLSSYVMGWVRQAPGKGLEWVGYIYGGGHTYYATWARGRFTISRDTSKNTVYLQMNSLRAEDTAVYFCARAAADFYFNLWGQGTLVTVSS

SEQ ID NO: 653 - protein, 117 aa, synthetic construct
  REGION 1..117: note = VH domain of 9460 IgG histidine scanning variant #18
EVQLVESGGGLVQPGGSLRLSCTASGIDLSSYVMGWVRQAPGKGLEWVGYIYGGGGHYYATWARGRFTISRDTSKNTVYLQMNSLRAEDTAVYFCARAAADFYFNLWGQGTLVTVSS

SEQ ID NO: 654 - protein, 117 aa, synthetic construct
  REGION 1..117: note = VH domain of 9460 IgG histidine scanning variant #19
EVQLVESGGGLVQPGGSLRLSCTASGIDLSSYVMGWVRQAPGKGLEWVGYIYGGGGTHYATWARGRFTISRDTSKNTVYLQMNSLRAEDTAVYFCARAAADFYFNLWGQGTLVTVSS

SEQ ID NO: 655 - protein, 117 aa, synthetic construct
  REGION 1..117: note = VH domain of 9460 IgG histidine scanning variant #20
EVQLVESGGGLVQPGGSLRLSCTASGIDLSSYVMGWVRQAPGKGLEWVGYIYGGGGTYHATWARGRFTISRDTSKNTVYLQMNSLRAEDTAVYFCARAAADFYFNLWGQGTLVTVSS

SEQ ID NO: 656 - protein, 117 aa, synthetic construct
  REGION 1..117: note = VH domain of 9460 IgG histidine scanning variant #21
EVQLVESGGGLVQPGGSLRLSCTASGIDLSSYVMGWVRQAPGKGLEWVGYIYGGGGTYYHTWARGRFTISRDTSKNTVYLQMNSLRAEDTAVYFCARAAADFYFNLWGQGTLVTVSS

SEQ ID NO: 657 - protein, 117 aa, synthetic construct
  REGION 1..117: note = VH domain of 9460 IgG histidine scanning variant #22
EVQLVESGGGLVQPGGSLRLSCTASGIDLSSYVMGWVRQAPGKGLEWVGYIYGGGGTYYAHWARGRFTISRDTSKNTVYLQMNSLRAEDTAVYFCARAAADFYFNLWGQGTLVTVSS

SEQ ID NO: 658 - protein, 117 aa, synthetic construct
  REGION 1..117: note = VH domain of 9460 IgG histidine scanning variant #23
EVQLVESGGGLVQPGGSLRLSCTASGIDLSSYVMGWVRQAPGKGLEWVGYIYGGGGTYYATHARGRFTISRDTSKNTVYLQMNSLRAEDTAVYFCARAAADFYFNLWGQGTLVTVSS

SEQ ID NO: 659 - protein, 117 aa, synthetic construct
  REGION 1..117: note = VH domain of 9460 IgG histidine scanning variant #24
EVQLVESGGGLVQPGGSLRLSCTASGIDLSSYVMGWVRQAPGKGLEWVGYIYGGGGTYYATWHRGRFTISRDTSKNTVYLQMNSLRAEDTAVYFCARAAADFYFNLWGQGTLVTVSS

SEQ ID NO: 660 - protein, 117 aa, synthetic construct
  REGION 1..117: note = VH domain of 9460 IgG histidine scanning variant #25
EVQLVESGGGLVQPGGSLRLSCTASGIDLSSYVMGWVRQAPGKGLEWVGYIYGGGGTYYATWAHGRFTISRDTSKNTVYLQMNSLRAEDTAVYFCARAAADFYFNLWGQGTLVTVSS

SEQ ID NO: 661 - protein, 117 aa, synthetic construct
  REGION 1..117: note = VH domain of 9460 IgG histidine scanning variant #26
EVQLVESGGGLVQPGGSLRLSCTASGIDLSSYVMGWVRQAPGKGLEWVGYIYGGGGTYYATWARHRFTISRDTSKNTVYLQMNSLRAEDTAVYFCARAAADFYFNLWGQGTLVTVSS

SEQ ID NO: 662 - protein, 117 aa, synthetic construct
  REGION 1..117: note = VH domain of 9460 IgG histidine scanning variant #27
EVQLVESGGGLVQPGGSLRLSCTASGIDLSSYVMGWVRQAPGKGLEWVGYIYGGGGTYYATWARGRFTISRDTSKNTVYLQMNSLRAEDTAVYFCHRAAADFYFNLWGQGTLVTVSS

SEQ ID NO: 663 - protein, 117 aa, synthetic construct
  REGION 1..117: note = VH domain of 9460 IgG histidine scanning variant #28
EVQLVESGGGLVQPGGSLRLSCTASGIDLSSYVMGWVRQAPGKGLEWVGYIYGGGGTYYATWARGRFTISRDTSKNTVYLQMNSLRAEDTAVYFCAHAAADFYFNLWGQGTLVTVSS

SEQ ID NO: 664 - protein, 117 aa, synthetic construct
  REGION 1..117: note = VH domain of 9460 IgG histidine scanning variant #29
EVQLVESGGGLVQPGGSLRLSCTASGIDLSSYVMGWVRQAPGKGLEWVGYIYGGGGTYYATWARGRFTISRDTSKNTVYLQMNSLRAEDTAVYFCARHAADFYFNLWGQGTLVTVSS

SEQ ID NO: 665 - protein, 117 aa, synthetic construct
  REGION 1..117: note = VH domain of 9460 IgG histidine scanning variant #30
EVQLVESGGGLVQPGGSLRLSCTASGIDLSSYVMGWVRQAPGKGLEWVGYIYGGGGTYYATWARGRFTISRDTSKNTVYLQMNSLRAEDTAVYFCARAHADFYFNLWGQGTLVTVSS

SEQ ID NO: 666 - protein, 117 aa, synthetic construct
  REGION 1..117: note = VH domain of 9460 IgG histidine scanning variant #31
EVQLVESGGGLVQPGGSLRLSCTASGIDLSSYVMGWVRQAPGKGLEWVGYIYGGGGTYYATWARGRFTISRDTSKNTVYLQMNSLRAEDTAVYFCARAAHDFYFNLWGQGTLVTVSS

SEQ ID NO: 667 - protein, 117 aa, synthetic construct
  REGION 1..117: note = VH domain of 9460 IgG histidine scanning variant #32
EVQLVESGGGLVQPGGSLRLSCTASGIDLSSYVMGWVRQAPGKGLEWVGYIYGGGGTYYATWARGRFTISRDTSKNTVYLQMNSLRAEDTAVYFCARAAAHFYFNLWGQGTLVTVSS

SEQ ID NO: 668 - protein, 117 aa, synthetic construct
  REGION 1..117: note = VH domain of 9460 IgG histidine scanning variant #33
EVQLVESGGGLVQPGGSLRLSCTASGIDLSSYVMGWVRQAPGKGLEWVGYIYGGGGTYYATWARGRFTISRDTSKNTVYLQMNSLRAEDTAVYFCARAAADHYFNLWGQGTLVTVSS

SEQ ID NO: 669 - protein, 117 aa, synthetic construct
  REGION 1..117: note = VH domain of 9460 IgG histidine scanning variant #34
EVQLVESGGGLVQPGGSLRLSCTASGIDLSSYVMGWVRQAPGKGLEWVGYIYGGGGTYYATWARGRFTISRDTSKNTVYLQMNSLRAEDTAVYFCARAAADFHFNLWGQGTLVTVSS

SEQ ID NO: 670 - protein, 117 aa, synthetic construct
  REGION 1..117: note = VH domain of 9460 IgG histidine scanning variant #35
EVQLVESGGGLVQPGGSLRLSCTASGIDLSSYVMGWVRQAPGKGLEWVGYIYGGGGTYYATWARGRFTISRDTSKNTVYLQMNSLRAEDTAVYFCARAAADFYHNLWGQGTLVTVSS

SEQ ID NO: 671 - protein, 117 aa, synthetic construct
  REGION 1..117: note = VH domain of 9460 IgG histidine scanning variant #36
EVQLVESGGGLVQPGGSLRLSCTASGIDLSSYVMGWVRQAPGKGLEWVGYIYGGGGTYYATWARGRFTISRDTSKNTVYLQMNSLRAEDTAVYFCARAAADFYFHLWGQGTLVTVSS

SEQ ID NO: 672 - protein, 117 aa, synthetic construct
  REGION 1..117: note = VH domain of 9460 IgG histidine scanning variant #37
EVQLVESGGGLVQPGGSLRLSCTASGIDLSSYVMGWVRQAPGKGLEWVGYIYGGGGTYYATWARGRFTISRDTSKNTVYLQMNSLRAEDTAVYFCARAAADFYFNHWGQGTLVTVSS

SEQ ID NO: 673 - protein, 117 aa, synthetic construct
  REGION 1..117: note = VH domain of 9460 IgG histidine scanning variant #38
EVQLVESGGGLVQPGGSLRLSCTASGIDLSSYVMGWVRQAPGKGLEWVGYIYGGGGTYYATWARGRFTISRDTSKNTVYLQMNSLRAEDTAVYYCARAAADFYFNLWGQGTLVTVSS

SEQ ID NO: 674 - protein, 117 aa, synthetic construct
  REGION 1..117: note = VH domain of 9460 IgG histidine scanning variant #39
EVQLVESGGGLVQPGGSLRLSCTASGIALSSYVMGWVRQAPGKGLEWVGYIYGGGGTYYATWARGRFTISRDTSKNTVYLQMNSLRAEDTAVYFCARAAADFYFNLWGQGTLVTVSS

SEQ ID NO: 675 - protein, 117 aa, synthetic construct
  REGION 1..117: note = VH domain of 9460 IgG histidine scanning variant #40
EVQLVESGGGLVQPGGSLRLSCTASGIDLSSYVMGWVRQAPGKGLEWVGYIYGGGGTYYATWARGRFTISRDTSKNTVYLQMNSLRAEDTAVYFCARAAAAFYFNLWGQGTLVTVSS

SEQ ID NO: 676 - protein, 110 aa, synthetic construct
  REGION 1..110: note = VL domain of 9460 IgG histidine scanning variant #1
EIVMTQSPSTLSASVGDRVIITCHASQSISSYLAWYQQKPGKAPKLLIYDISDLASGVPSRFSGSGSGAEFTLTISSLQPDDFATYYCQSYYWISNSGAAFGQGTKLTVL

SEQ ID NO: 677 - protein, 110 aa, synthetic construct
  REGION 1..110: note = VL domain of 9460 IgG histidine scanning variant #2
EIVMTQSPSTLSASVGDRVIITCQHSQSISSYLAWYQQKPGKAPKLLIYDISDLASGVPSRFSGSGSGAEFTLTISSLQPDDFATYYCQSYYWISNSGAAFGQGTKLTVL

SEQ ID NO: 678 - protein, 110 aa, synthetic construct
  REGION 1..110: note = VL domain of 9460 IgG histidine scanning variant #3
EIVMTQSPSTLSASVGDRVIITCQAHQSISSYLAWYQQKPGKAPKLLIYDISDLASGVPSRFSGSGSGAEFTLTISSLQPDDFATYYCQSYYWISNSGAAFGQGTKLTVL

SEQ ID NO: 679 - protein, 110 aa, synthetic construct
  REGION 1..110: note = VL domain of 9460 IgG histidine scanning variant #4
EIVMTQSPSTLSASVGDRVIITCQASHSISSYLAWYQQKPGKAPKLLIYDISDLASGVPSRFSGSGSGAEFTLTISSLQPDDFATYYCQSYYWISNSGAAFGQGTKLTVL

SEQ ID NO: 680 - protein, 110 aa, synthetic construct
  REGION 1..110: note = VL domain of 9460 IgG histidine scanning variant #5
EIVMTQSPSTLSASVGDRVIITCQASQHISSYLAWYQQKPGKAPKLLIYDISDLASGVPSRFSGSGSGAEFTLTISSLQPDDFATYYCQSYYWISNSGAAFGQGTKLTVL

SEQ ID NO: 681 - protein, 110 aa, synthetic construct
  REGION 1..110: note = VL domain of 9460 IgG histidine scanning variant #6
EIVMTQSPSTLSASVGDRVIITCQASQSHSSYLAWYQQKPGKAPKLLIYDISDLASGVPSRFSGSGSGAEFTLTISSLQPDDFATYYCQSYYWISNSGAAFGQGTKLTVL

SEQ ID NO: 682 - protein, 110 aa, synthetic construct
  REGION 1..110: note = VL domain of 9460 IgG histidine scanning variant #7
EIVMTQSPSTLSASVGDRVIITCQASQSIHSYLAWYQQKPGKAPKLLIYDISDLASGVPSRFSGSGSGAEFTLTISSLQPDDFATYYCQSYYWISNSGAAFGQGTKLTVL

SEQ ID NO: 683 - protein, 110 aa, synthetic construct
  REGION 1..110: note = VL domain of 9460 IgG histidine scanning variant #8
EIVMTQSPSTLSASVGDRVIITCQASQSISHYLAWYQQKPGKAPKLLIYDISDLASGVPSRFSGSGSGAEFTLTISSLQPDDFATYYCQSYYWISNSGAAFGQGTKLTVL

SEQ ID NO: 684 - protein, 110 aa, synthetic construct
  REGION 1..110: note = VL domain of 9460 IgG histidine scanning variant #9
EIVMTQSPSTLSASVGDRVIITCQASQSISSHLAWYQQKPGKAPKLLIYDISDLASGVPSRFSGSGSGAEFTLTISSLQPDDFATYYCQSYYWISNSGAAFGQGTKLTVL

SEQ ID NO: 685 - protein, 110 aa, synthetic construct
  REGION 1..110: note = VL domain of 9460 IgG histidine scanning variant #10
EIVMTQSPSTLSASVGDRVIITCQASQSISSYHAWYQQKPGKAPKLLIYDISDLASGVPSRFSGSGSGAEFTLTISSLQPDDFATYYCQSYYWISNSGAAFGQGTKLTVL

SEQ ID NO: 686 - protein, 110 aa, synthetic construct
  REGION 1..110: note = VL domain of 9460 IgG histidine scanning variant #11
EIVMTQSPSTLSASVGDRVIITCQASQSISSYLHWYQQKPGKAPKLLIYDISDLASGVPSRFSGSGSGAEFTLTISSLQPDDFATYYCQSYYWISNSGAAFGQGTKLTVL

SEQ ID NO: 687 - protein, 110 aa, synthetic construct
  REGION 1..110: note = VL domain of 9460 IgG histidine scanning variant #12
EIVMTQSPSTLSASVGDRVIITCQASQSISSYLAWYQQKPGKAPKLLIYHISDLASGVPSRFSGSGSGAEFTLTISSLQPDDFATYYCQSYYWISNSGAAFGQGTKLTVL

SEQ ID NO: 688 - protein, 110 aa, synthetic construct
  REGION 1..110: note = VL domain of 9460 IgG histidine scanning variant #13
EIVMTQSPSTLSASVGDRVIITCQASQSISSYLAWYQQKPGKAPKLLIYDHSDLASGVPSRFSGSGSGAEFTLTISSLQPDDFATYYCQSYYWISNSGAAFGQGTKLTVL

SEQ ID NO: 689 - protein, 110 aa, synthetic construct
  REGION 1..110: note = VL domain of 9460 IgG histidine scanning variant #14
EIVMTQSPSTLSASVGDRVIITCQASQSISSYLAWYQQKPGKAPKLLIYDIHDLASGVPSRFSGSGSGAEFTLTISSLQPDDFATYYCQSYYWISNSGAAFGQGTKLTVL

SEQ ID NO: 690 - protein, 110 aa, synthetic construct
  REGION 1..110: note = VL domain of 9460 IgG histidine scanning variant #15
EIVMTQSPSTLSASVGDRVIITCQASQSISSYLAWYQQKPGKAPKLLIYDISHLASGVPSRFSGSGSGAEFTLTISSLQPDDFATYYCQSYYWISNSGAAFGQGTKLTVL

SEQ ID NO: 691 - protein, 110 aa, synthetic construct
  REGION 1..110: note = VL domain of 9460 IgG histidine scanning variant #16
EIVMTQSPSTLSASVGDRVIITCQASQSISSYLAWYQQKPGKAPKLLIYDISDHASGVPSRFSGSGSGAEFTLTISSLQPDDFATYYCQSYYWISNSGAAFGQGTKLTVL

SEQ ID NO: 692 - protein, 110 aa, synthetic construct
  REGION 1..110: note = VL domain of 9460 IgG histidine scanning variant #17
EIVMTQSPSTLSASVGDRVIITCQASQSISSYLAWYQQKPGKAPKLLIYDISDLHSGVPSRFSGSGSGAEFTLTISSLQPDDFATYYCQSYYWISNSGAAFGQGTKLTVL

SEQ ID NO: 693 - protein, 110 aa, synthetic construct
  REGION 1..110: note = VL domain of 9460 IgG histidine scanning variant #18
EIVMTQSPSTLSASVGDRVIITCQASQSISSYLAWYQQKPGKAPKLLIYDISDLAHGVPSRFSGSGSGAEFTLTISSLQPDDFATYYCQSYYWISNSGAAFGQGTKLTVL

SEQ ID NO: 694 - protein, 110 aa, synthetic construct
  REGION 1..110: note = VL domain of 9460 IgG histidine scanning variant #19
EIVMTQSPSTLSASVGDRVIITCQASQSISSYLAWYQQKPGKAPKLLIYDISDLASGVPSRFSGSGSGAEFTLTISSLQPDDFATYYCHSYYWISNSGAAFGQGTKLTVL

SEQ ID NO: 695 - protein, 110 aa, synthetic construct
  REGION 1..110: note = VL domain of 9460 IgG histidine scanning variant #20
EIVMTQSPSTLSASVGDRVIITCQASQSISSYLAWYQQKPGKAPKLLIYDISDLASGVPSRFSGSGSGAEFTLTISSLQPDDFATYYCQHYYWISNSGAAFGQGTKLTVL

SEQ ID NO: 696 - protein, 110 aa, synthetic construct
  REGION 1..110: note = VL domain of 9460 IgG histidine scanning variant #21
EIVMTQSPSTLSASVGDRVIITCQASQSISSYLAWYQQKPGKAPKLLIYDISDLASGVPSRFSGSGSGAEFTLTISSLQPDDFATYYCQSHYWISNSGAAFGQGTKLTVL

SEQ ID NO: 697 - protein, 110 aa, synthetic construct
  REGION 1..110: note = VL domain of 9460 IgG histidine scanning variant #22
EIVMTQSPSTLSASVGDRVIITCQASQSISSYLAWYQQKPGKAPKLLIYDISDLASGVPSRFSGSGSGAEFTLTISSLQPDDFATYYCQSYHWISNSGAAFGQGTKLTVL

SEQ ID NO: 698 - protein, 110 aa, synthetic construct
  REGION 1..110: note = VL domain of 9460 IgG histidine scanning variant #23
EIVMTQSPSTLSASVGDRVIITCQASQSISSYLAWYQQKPGKAPKLLIYDISDLASGVPSRFSGSGSGAEFTLTISSLQPDDFATYYCQSYYHISNSGAAFGQGTKLTVL

SEQ ID NO: 699 - protein, 110 aa, synthetic construct
  REGION 1..110: note = VL domain of 9460 IgG histidine scanning variant #24
EIVMTQSPSTLSASVGDRVIITCQASQSISSYLAWYQQKPGKAPKLLIYDISDLASGVPSRFSGSGSGAEFTLTISSLQPDDFATYYCQSYYWHSNSGAAFGQGTKLTVL

SEQ ID NO: 700 - protein, 110 aa, synthetic construct
  REGION 1..110: note = VL domain of 9460 IgG histidine scanning variant #25
EIVMTQSPSTLSASVGDRVIITCQASQSISSYLAWYQQKPGKAPKLLIYDISDLASGVPSRFSGSGSGAEFTLTISSLQPDDFATYYCQSYYWIHNSGAAFGQGTKLTVL

SEQ ID NO: 701 - protein, 110 aa, synthetic construct
  REGION 1..110: note = VL domain of 9460 IgG histidine scanning variant #26
EIVMTQSPSTLSASVGDRVIITCQASQSISSYLAWYQQKPGKAPKLLIYDISDLASGVPSRFSGSGSGAEFTLTISSLQPDDFATYYCQSYYWISHSGAAFGQGTKLTVL

SEQ ID NO: 702 - protein, 110 aa, synthetic construct
  REGION 1..110: note = VL domain of 9460 IgG histidine scanning variant #27
EIVMTQSPSTLSASVGDRVIITCQASQSISSYLAWYQQKPGKAPKLLIYDISDLASGVPSRFSGSGSGAEFTLTISSLQPDDFATYYCQSYYWISNHGAAFGQGTKLTVL

SEQ ID NO: 703 - protein, 110 aa, synthetic construct
  REGION 1..110: note = VL domain of 9460 IgG histidine scanning variant #28
EIVMTQSPSTLSASVGDRVIITCQASQSISSYLAWYQQKPGKAPKLLIYDISDLASGVPSRFSGSGSGAEFTLTISSLQPDDFATYYCQSYYWISNSHAAFGQGTKLTVL

SEQ ID NO: 704 - protein, 110 aa, synthetic construct
  REGION 1..110: note = VL domain of 9460 IgG histidine scanning variant #29
EIVMTQSPSTLSASVGDRVIITCQASQSISSYLAWYQQKPGKAPKLLIYDISDLASGVPSRFSGSGSGAEFTLTISSLQPDDFATYYCQSYYWISNSGHAFGQGTKLTVL

SEQ ID NO: 705 - protein, 110 aa, synthetic construct
  REGION 1..110: note = VL domain of 9460 IgG histidine scanning variant #30
EIVMTQSPSTLSASVGDRVIITCQASQSISSYLAWYQQKPGKAPKLLIYDISDLASGVPSRFSGSGSGAEFTLTISSLQPDDFATYYCQSYYWISNSGAHFGQGTKLTVL

SEQ ID NO: 706 - protein, 110 aa, synthetic construct
  REGION 1..110: note = VL domain of 9460 IgG histidine scanning variant #31
EIVMTQSPSTLSASVGDRVIITCQASQSISSYLAWYQQKPGKAPKLLIYDISDLASGVPSRFSGSGSGAEFTLTISSLQPDDFATYYCQSYYWISNSGAAFGQGTKLEIK

SEQ ID NO: 707 - protein, 110 aa, synthetic construct
  REGION 1..110: note = VL domain of 9460 IgG histidine scanning variant #32
EIVMTQSPSTLSASVGDRVIITCQASQSISSYLAWYQQKPGKAPKLLIYAISDLASGVPSRFSGSGSGAEFTLTISSLQPDDFATYYCQSYYWISNSGAAFGQGTKLTVL

SEQ ID NO: 708 - protein, 110 aa, synthetic construct
  REGION 1..110: note = VL domain of 9460 IgG histidine scanning variant #33
EIVMTQSPSTLSASVGDRVIITCQASQSISSYLAWYQQKPGKAPKLLIYDISALASGVPSRFSGSGSGAEFTLTISSLQPDDFATYYCQSYYWISNSGAAFGQGTKLTVL

SEQ ID NO: 709 - protein, 110 aa, synthetic construct
  REGION 1..110: note = VL domain of 9460 IgG histidine scanning variant #34
EIVMTQSPSTLSASVGDRVIITCQASQSISSYLAWYQQKPGKAPKLLIYDISDLASGVPSRFSGSGSGAEFTLTISSLQPDDFATYYCQSYYWISASGAAFGQGTKLTVL

SEQ ID NO: 710 - protein, 126 aa, synthetic construct
  REGION 1..126: note = VH domain of 9792 IgG
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPGYSGYGYAFDAFDPWGQGTLVTVSS

SEQ ID NO: 711 - protein, 111 aa, synthetic construct
  REGION 1..111: note = VL domain of 9792 IgG
EIVMTQSPSTLSASVGDRVIITCQSSKSVYNDNWLSWYQQKPGKAPKLLIYGASTLASGVPSRFSGSGSGAEFTLTISSLQPDDFATYYCAGGYSSSSDNGFGQGTKLTVL

SEQ ID NO: 712 - protein, 10 aa, synthetic construct
  REGION 1..10: note = Heavy Chain CDR1 of 9792 IgG
GIDLSRYSMG

SEQ ID NO: 713 - protein, 16 aa, synthetic construct
  REGION 1..16: note = Heavy Chain CDR2 of 9792 IgG
IISYGGSTYYASWAKG

SEQ ID NO: 714 - protein, 20 aa, synthetic construct
  REGION 1..20: note = Heavy Chain CDR3 of 9792 IgG
ARVGYPGYSGYGYAFDAFDP

SEQ ID NO: 715 - protein, 13 aa, synthetic construct
  REGION 1..13: note = Light Chain CDR1 of 9792 IgG
QSSKSVYNDNWLS

SEQ ID NO: 716 - protein, 7 aa, synthetic construct
  REGION 1..7: note = Light Chain CDR2 of 9792 IgG
GASTLAS

SEQ ID NO: 717 - protein, 11 aa, synthetic construct
  REGION 1..11: note = Light Chain CDR3 of 9792 IgG
AGGYSSSSDNG

SEQ ID NO: 718 - protein, 126 aa, synthetic construct
  REGION 1..126: note = VH domain of 9792 IgG histidine scanning variant #1
EVQLVESGGGLVQPGGSLRLSCTASHIDLSRYSMGWVRQAPGKGLEWVGIISYGGSTYYASWAKGRFTISRDTSKNTVYLQMNSLRAEDTAVYFCARVGYPGYSGYGYAFDAFDPWGQGTLVTVSS

SEQ ID NO: 719 - protein, 126 aa, synthetic construct
  REGION 1..126: note = VH domain of 9792 IgG histidine scanning variant #2
EVQLVESGGGLVQPGGSLRLSCTASGHDLSRYSMGWVRQAPGKGLEWVGIISYGGSTYYASWAKGRFTISRDTSKNTVYLQMNSLRAEDTAVYFCARVGYPGYSGYGYAFDAFDPWGQGTLVTVSS

SEQ ID NO: 720 - protein, 126 aa, synthetic construct
  REGION 1..126: note = VH domain of 9792 IgG histidine scanning variant #3
EVQLVESGGGLVQPGGSLRLSCTASGIHLSRYSMGWVRQAPGKGLEWVGIISYGGSTYYASWAKGRFTISRDTSKNTVYLQMNSLRAEDTAVYFCARVGYPGYSGYGYAFDAFDPWGQGTLVTVSS

SEQ ID NO: 721 - protein, 126 aa, synthetic construct
  REGION 1..126: note = VH domain of 9792 IgG histidine scanning variant #4
EVQLVESGGGLVQPGGSLRLSCTASGIDHSRYSMGWVRQAPGKGLEWVGIISYGGSTYYASWAKGRFTISRDTSKNTVYLQMNSLRAEDTAVYFCARVGYPGYSGYGYAFDAFDPWGQGTLVTVSS

SEQ ID NO: 722 - protein, 126 aa, synthetic construct
  REGION 1..126: note = VH domain of 9792 IgG histidine scanning variant #5
EVQLVESGGGLVQPGGSLRLSCTASGIDLHRYSMGWVRQAPGKGLEWVGIISYGGSTYYASWAKGRFTISRDTSKNTVYLQMNSLRAEDTAVYFCARVGYPGYSGYGYAFDAFDPWGQGTLVTVSS

SEQ ID NO: 723 - protein, 126 aa, synthetic construct
  REGION 1..126: note = VH domain of 9792 IgG histidine scanning variant #6
EVQLVESGGGLVQPGGSLRLSCTASGIDLSHYSMGWVRQAPGKGLEWVGIISYGGSTYYASWAKGRFTISRDTSKNTVYLQMNSLRAEDTAVYFCARVGYPGYSGYGYAFDAFDPWGQGTLVTVSS

SEQ ID NO: 724 - protein, 126 aa, synthetic construct
  REGION 1..126: note = VH domain of 9792 IgG histidine scanning variant #7
EVQLVESGGGLVQPGGSLRLSCTASGIDLSRHSMGWVRQAPGKGLEWVGIISYGGSTYYASWAKGRFTISRDTSKNTVYLQMNSLRAEDTAVYFCARVGYPGYSGYGYAFDAFDPWGQGTLVTVSS

SEQ ID NO: 725 - protein, 126 aa, synthetic construct
  REGION 1..126: note = VH domain of 9792 IgG histidine scanning variant #8
EVQLVESGGGLVQPGGSLRLSCTASGIDLSRYHMGWVRQAPGKGLEWVGIISYGGSTYYASWAKGRFTISRDTSKNTVYLQMNSLRAEDTAVYFCARVGYPGYSGYGYAFDAFDPWGQGTLVTVSS

SEQ ID NO: 726 - protein, 126 aa, synthetic construct
  REGION 1..126: note = VH domain of 9792 IgG histidine scanning variant #9
EVQLVESGGGLVQPGGSLRLSCTASGIDLSRYSHGWVRQAPGKGLEWVGIISYGGSTYYASWAKGRFTISRDTSKNTVYLQMNSLRAEDTAVYFCARVGYPGYSGYGYAFDAFDPWGQGTLVTVSS

SEQ ID NO: 727 - protein, 126 aa, synthetic construct
  REGION 1..126: note = VH domain of 9792 IgG histidine scanning variant #10
EVQLVESGGGLVQPGGSLRLSCTASGIDLSRYSMHWVRQAPGKGLEWVGIISYGGSTYYASWAKGRFTISRDTSKNTVYLQMNSLRAEDTAVYFCARVGYPGYSGYGYAFDAFDPWGQGTLVTVSS

SEQ ID NO: 728 - protein, 126 aa, synthetic construct
  REGION 1..126: note = VH domain of 9792 IgG histidine scanning variant #11
EVQLVESGGGLVQPGGSLRLSCTASGIDLSRYSMGWVRQAPGKGLEWVGHISYGGSTYYASWAKGRFTISRDTSKNTVYLQMNSLRAEDTAVYFCARVGYPGYSGYGYAFDAFDPWGQGTLVTVSS

SEQ ID NO: 729 - protein, 126 aa, synthetic construct
  REGION 1..126: note = VH domain of 9792 IgG histidine scanning variant #12
EVQLVESGGGLVQPGGSLRLSCTASGIDLSRYSMGWVRQAPGKGLEWVGIHSYGGSTYYASWAKGRFTISRDTSKNTVYLQMNSLRAEDTAVYFCARVGYPGYSGYGYAFDAFDPWGQGTLVTVSS

SEQ ID NO: 730 - protein, 126 aa, synthetic construct
  REGION 1..126: note = VH domain of 9792 IgG histidine scanning variant #13
EVQLVESGGGLVQPGGSLRLSCTASGIDLSRYSMGWVRQAPGKGLEWVGIIHYGGSTYYASWAKGRFTISRDTSKNTVYLQMNSLRAEDTAVYFCARVGYPGYSGYGYAFDAFDPWGQGTLVTVSS

SEQ ID NO: 731 - protein, 126 aa, synthetic construct
  REGION 1..126: note = VH domain of 9792 IgG histidine scanning variant #14
EVQLVESGGGLVQPGGSLRLSCTASGIDLSRYSMGWVRQAPGKGLEWVGIISHGGSTYYASWAKGRFTISRDTSKNTVYLQMNSLRAEDTAVYFCARVGYPGYSGYGYAFDAFDPWGQGTLVTVSS

SEQ ID NO: 732 - protein, 126 aa, synthetic construct
  REGION 1..126: note = VH domain of 9792 IgG histidine scanning variant #15
EVQLVESGGGLVQPGGSLRLSCTASGIDLSRYSMGWVRQAPGKGLEWVGIISYHGSTYYASWAKGRFTISRDTSKNTVYLQMNSLRAEDTAVYFCARVGYPGYSGYGYAFDAFDPWGQGTLVTVSS

SEQ ID NO: 733 - protein, 126 aa, synthetic construct
  REGION 1..126: note = VH domain of 9792 IgG histidine scanning variant #16
EVQLVESGGGLVQPGGSLRLSCTASGIDLSRYSMGWVRQAPGKGLEWVGIISYGHSTYYASWAKGRFTISRDTSKNTVYLQMNSLRAEDTAVYFCARVGYPGYSGYGYAFDAFDPWGQGTLVTVSS

SEQ ID NO: 734 - protein, 126 aa, synthetic construct
  REGION 1..126: note = VH domain of 9792 IgG histidine scanning variant #17
EVQLVESGGGLVQPGGSLRLSCTASGIDLSRYSMGWVRQAPGKGLEWVGIISYGGHTYYASWAKGRFTISRDTSKNTVYLQMNSLRAEDTAVYFCARVGYPGYSGYGYAFDAFDPWGQGTLVTVSS

SEQ ID NO: 735 - protein, 126 aa, synthetic construct
  REGION 1..126: note = VH domain of 9792 IgG histidine scanning variant #18
EVQLVESGGGLVQPGGSLRLSCTASGIDLSRYSMGWVRQAPGKGLEWVGIISYGGSHYYASWAKGRFTISRDTSKNTVYLQMNSLRAEDTAVYFCARVGYPGYSGYGYAFDAFDPWGQGTLVTVSS

SEQ ID NO: 736 - protein, 126 aa, synthetic construct
  REGION 1..126: note = VH domain of 9792 IgG histidine scanning variant #19
EVQLVESGGGLVQPGGSLRLSCTASGIDLSRYSMGWVRQAPGKGLEWVGIISYGGSTHYASWAKGRFTISRDTSKNTVYLQMNSLRAEDTAVYFCARVGYPGYSGYGYAFDAFDPWGQGTLVTVSS

SEQ ID NO: 737 - protein, 126 aa, synthetic construct
  REGION 1..126: note = VH domain of 9792 IgG histidine scanning variant #20
EVQLVESGGGLVQPGGSLRLSCTASGIDLSRYSMGWVRQAPGKGLEWVGIISYGGSTYHASWAKGRFTISRDTSKNTVYLQMNSLRAEDTAVYFCARVGYPGYSGYGYAFDAFDPWGQGTLVTVSS

SEQ ID NO: 738 - protein, 126 aa, synthetic construct
  REGION 1..126: note = VH domain of 9792 IgG histidine scanning variant #21
EVQLVESGGGLVQPGGSLRLSCTASGIDLSRYSMGWVRQAPGKGLEWVGIISYGGSTYYHSWAKGRFTISRDTSKNTVYLQMNSLRAEDTAVYFCARVGYPGYSGYGYAFDAFDPWGQGTLVTVSS

SEQ ID NO: 739 - protein, 126 aa, synthetic construct
  REGION 1..126: note = VH domain of 9792 IgG histidine scanning variant #22
EVQLVESGGGLVQPGGSLRLSCTASGIDLSRYSMGWVRQAPGKGLEWVGIISYGGSTYYAHWAKGRFTISRDTSKNTVYLQMNSLRAEDTAVYFCARVGYPGYSGYGYAFDAFDPWGQGTLVTVSS

SEQ ID NO: 740 - protein, 126 aa, synthetic construct
  REGION 1..126: note = VH domain of 9792 IgG histidine scanning variant #23
EVQLVESGGGLVQPGGSLRLSCTASGIDLSRYSMGWVRQAPGKGLEWVGIISYGGSTYYASHAKGRFTISRDTSKNTVYLQMNSLRAEDTAVYFCARVGYPGYSGYGYAFDAFDPWGQGTLVTVSS

SEQ ID NO: 741 - protein, 126 aa, synthetic construct
  REGION 1..126: note = VH domain of 9792 IgG histidine scanning variant #24
EVQLVESGGGLVQPGGSLRLSCTASGIDLSRYSMGWVRQAPGKGLEWVGIISYGGSTYYASWHKGRFTISRDTSKNTVYLQMNSLRAEDTAVYFCARVGYPGYSGYGYAFDAFDPWGQGTLVTVSS

SEQ ID NO: 742 - protein, 126 aa, synthetic construct
  REGION 1..126: note = VH domain of 9792 IgG histidine scanning variant #25
EVQLVESGGGLVQPGGSLRLSCTASGIDLSRYSMGWVRQAPGKGLEWVGIISYGGSTYYASWAHGRFTISRDTSKNTVYLQMNSLRAEDTAVYFCARVGYPGYSGYGYAFDAFDPWGQGTLVTVSS

SEQ ID NO: 743 - protein, 126 aa, synthetic construct
  REGION 1..126: note = VH domain of 9792 IgG histidine scanning variant #26
EVQLVESGGGLVQPGGSLRLSCTASGIDLSRYSMGWVRQAPGKGLEWVGIISYGGSTYYASWAKHRFTISRDTSKNTVYLQMNSLRAEDTAVYFCARVGYPGYSGYGYAFDAFDPWGQGTLVTVSS

SEQ ID NO: 744 - protein, 126 aa, synthetic construct
  REGION 1..126: note = VH domain of 9792 IgG histidine scanning variant #27
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCHRVGYPGYSGYGYAFDAFDPWGQGTLVTVSS

SEQ ID NO: 745 - protein, 126 aa, synthetic construct
  REGION 1..126: note = VH domain of 9792 IgG histidine scanning variant #28
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCAHVGYPGYSGYGYAFDAFDPWGQGTLVTVSS

SEQ ID NO: 746 - protein, 126 aa, synthetic construct
  REGION 1..126: note = VH domain of 9792 IgG histidine scanning variant #29
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARHGYPGYSGYGYAFDAFDPWGQGTLVTVSS

SEQ ID NO: 747 - protein, 126 aa, synthetic construct
  REGION 1..126: note = VH domain of 9792 IgG histidine scanning variant #30
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVHYPGYSGYGYAFDAFDPWGQGTLVTVSS

SEQ ID NO: 748 - protein, 126 aa, synthetic construct
  REGION 1..126: note = VH domain of 9792 IgG histidine scanning variant #31
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGHPGYSGYGYAFDAFDPWGQGTLVTVSS

SEQ ID NO: 749 - protein, 126 aa, synthetic construct
  REGION 1..126: note = VH domain of 9792 IgG histidine scanning variant #32
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYHGYSGYGYAFDAFDPWGQGTLVTVSS

SEQ ID NO: 750 - protein, 126 aa, synthetic construct
  REGION 1..126: note = VH domain of 9792 IgG histidine scanning variant #33
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPHYSGYGYAFDAFDPWGQGTLVTVSS

SEQ ID NO: 751 - protein, 126 aa, synthetic construct
  REGION 1..126: note = VH domain of 9792 IgG histidine scanning variant #34
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPGHSGYGYAFDAFDPWGQGTLVTVSS

SEQ ID NO: 752 - protein, 126 aa, synthetic construct
  REGION 1..126: note = VH domain of 9792 IgG histidine scanning variant #35
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPGYHGYGYAFDAFDPWGQGTLVTVSS

SEQ ID NO: 753 - protein, 126 aa, synthetic construct
  REGION 1..126: note = VH domain of 9792 IgG histidine scanning variant #36
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPGYSHYGYAFDAFDPWGQGTLVTVSS

SEQ ID NO: 754 - protein, 126 aa, synthetic construct
  REGION 1..126: note = VH domain of 9792 IgG histidine scanning variant #37
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPGYSGHGYAFDAFDPWGQGTLVTVSS

SEQ ID NO: 755 - protein, 126 aa, synthetic construct
  REGION 1..126: note = VH domain of 9792 IgG histidine scanning variant #38
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPGYSGYHYAFDAFDPWGQGTLVTVSS

SEQ ID NO: 756 - protein, 126 aa, synthetic construct
  REGION 1..126: note = VH domain of 9792 IgG histidine scanning variant #39
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPGYSGYGHAFDAFDPWGQGTLVTVSS

SEQ ID NO: 757 - protein, 126 aa, synthetic construct
  REGION 1..126: note = VH domain of 9792 IgG histidine scanning variant #40
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPGYSGYGYHFDAFDPWGQGTLVTVSS

SEQ ID NO: 758 - protein, 126 aa, synthetic construct
  REGION 1..126: note = VH domain of 9792 IgG histidine scanning variant #41
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPGYSGYGYAHDAFDPWGQGTLVTVSS

SEQ ID NO: 759 - protein, 126 aa, synthetic construct
  REGION 1..126: note = VH domain of 9792 IgG histidine scanning variant #42
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPGYSGYGYAFHAFDPWGQGTLVTVSS

SEQ ID NO: 760 - protein, 126 aa, synthetic construct
  REGION 1..126: note = VH domain of 9792 IgG histidine scanning variant #43
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPGYSGYGYAFDHFDPWGQGTLVTVSS

SEQ ID NO: 761 - protein, 126 aa, synthetic construct
  REGION 1..126: note = VH domain of 9792 IgG histidine scanning variant #44
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPGYSGYGYAFDAHDPWGQGTLVTVSS

SEQ ID NO: 762 - protein, 126 aa, synthetic construct
  REGION 1..126: note = VH domain of 9792 IgG histidine scanning variant #45
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPGYSGYGYAFDAFHPWGQGTLVTVSS

SEQ ID NO: 763 - protein, 126 aa, synthetic construct
  REGION 1..126: note = VH domain of 9792 IgG histidine scanning variant #46
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPGYSGYGYAFDAFDHWGQGTLVTVSS

SEQ ID NO: 764 - protein, 126 aa, synthetic construct
  REGION 1..126: note = VH domain of 9792 IgG histidine scanning variant #47
EVQLVESGGGLVQPGGSLRLSCTASGIDLSRYSMGWVRQAPGKGLEWVGIISYGGSTYYASWAKGRFTISRDTSKNTVYLQMNSLRAEDTAVYYCARVGYPGYSGYGYAFDAFDPWGQGTLVTVSS

SEQ ID NO: 765 - protein, 126 aa, synthetic construct
  REGION 1..126: note = VH domain of 9792 IgG histidine scanning variant #48
EVQLVESGGGLVQPGGSLRLSCTASGIALSRYSMGWVRQAPGKGLEWVGIISYGGSTYYASWAKGRFTISRDTSKNTVYLQMNSLRAEDTAVYFCARVGYPGYSGYGYAFDAFDPWGQGTLVTVSS

SEQ ID NO: 766 - protein, 126 aa, synthetic construct
  REGION 1..126: note = VH domain of 9792 IgG histidine scanning variant #49
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPGYSGYGYAFAAFDPWGQGTLVTVSS

SEQ ID NO: 767 - protein, 126 aa, synthetic construct
  REGION 1..126: note = VH domain of 9792 IgG histidine scanning variant #50
EVQLVESGGGLVQPGGSLRLSCTASGIDLSRYSMGWVRQAPGKGLEWVGIISYGGSTYYASWAKGRFTISRDTSKNTVYLQMNSLRAEDTAVYFCARVGYPGYSGYGYAFDAFAPWGQGTLVTVSS

SEQ ID NO: 768 - protein, 111 aa, synthetic construct
  REGION 1..111: note = VL domain of 9792 IgG histidine scanning variant #1
EIVMTQSPSTLSASVGDRVIITCHSSKSVYNDNWLSWYQQKPGKAPKLLIYGASTLASGVPSRFSGSGSGAEFTLTISSLQPDDFATYYCAGGYSSSSDNGFGQGTKLTVL

SEQ ID NO: 769 - protein, 111 aa, synthetic construct
  REGION 1..111: note = VL domain of 9792 IgG histidine scanning variant #2
EIVMTQSPSTLSASVGDRVIITCQHSKSVYNDNWLSWYQQKPGKAPKLLIYGASTLASGVPSRFSGSGSGAEFTLTISSLQPDDFATYYCAGGYSSSSDNGFGQGTKLTVL

SEQ ID NO: 770 - protein, 111 aa, synthetic construct
  REGION 1..111: note = VL domain of 9792 IgG histidine scanning variant #3
EIVMTQSPSTLSASVGDRVIITCQSHKSVYNDNWLSWYQQKPGKAPKLLIYGASTLASGVPSRFSGSGSGAEFTLTISSLQPDDFATYYCAGGYSSSSDNGFGQGTKLTVL

SEQ ID NO: 771 - protein, 111 aa, synthetic construct
  REGION 1..111: note = VL domain of 9792 IgG histidine scanning variant #4
EIVMTQSPSTLSASVGDRVIITCQSSHSVYNDNWLSWYQQKPGKAPKLLIYGASTLASGVPSRFSGSGSGAEFTLTISSLQPDDFATYYCAGGYSSSSDNGFGQGTKLTVL

SEQ ID NO: 772 - protein, 111 aa, synthetic construct
  REGION 1..111: note = VL domain of 9792 IgG histidine scanning variant #5
EIVMTQSPSTLSASVGDRVIITCQSSKHVYNDNWLSWYQQKPGKAPKLLIYGASTLASGVPSRFSGSGSGAEFTLTISSLQPDDFATYYCAGGYSSSSDNGFGQGTKLTVL

SEQ ID NO: 773 - protein, 111 aa, synthetic construct
  REGION 1..111: note = VL domain of 9792 IgG histidine scanning variant #6
EIVMTQSPSTLSASVGDRVIITCQSSKSHYNDNWLSWYQQKPGKAPKLLIYGASTLASGVPSRFSGSGSGAEFTLTISSLQPDDFATYYCAGGYSSSSDNGFGQGTKLTVL

SEQ ID NO: 774 - protein, 111 aa, synthetic construct
  REGION 1..111: note = VL domain of 9792 IgG histidine scanning variant #7
EIVMTQSPSTLSASVGDRVIITCQSSKSVHNDNWLSWYQQKPGKAPKLLIYGASTLASGVPSRFSGSGSGAEFTLTISSLQPDDFATYYCAGGYSSSSDNGFGQGTKLTVL

SEQ ID NO: 775 - protein, 111 aa, synthetic construct
  REGION 1..111: note = VL domain of 9792 IgG histidine scanning variant #8
EIVMTQSPSTLSASVGDRVIITCQSSKSVYHDNWLSWYQQKPGKAPKLLIYGASTLASGVPSRFSGSGSGAEFTLTISSLQPDDFATYYCAGGYSSSSDNGFGQGTKLTVL

SEQ ID NO: 776 - protein, 111 aa, synthetic construct
  REGION 1..111: note = VL domain of 9792 IgG histidine scanning variant #9
EIVMTQSPSTLSASVGDRVIITCQSSKSVYNHNWLSWYQQKPGKAPKLLIYGASTLASGVPSRFSGSGSGAEFTLTISSLQPDDFATYYCAGGYSSSSDNGFGQGTKLTVL

SEQ ID NO: 777 - protein, 111 aa, synthetic construct
  REGION 1..111: note = VL domain of 9792 IgG histidine scanning variant #10
EIVMTQSPSTLSASVGDRVIITCQSSKSVYNDHWLSWYQQKPGKAPKLLIYGASTLASGVPSRFSGSGSGAEFTLTISSLQPDDFATYYCAGGYSSSSDNGFGQGTKLTVL

SEQ ID NO: 778 - protein, 111 aa, synthetic construct
  REGION 1..111: note = VL domain of 9792 IgG histidine scanning variant #11
EIVMTQSPSTLSASVGDRVIITCQSSKSVYNDNHLSWYQQKPGKAPKLLIYGASTLASGVPSRFSGSGSGAEFTLTISSLQPDDFATYYCAGGYSSSSDNGFGQGTKLTVL

SEQ ID NO: 779 - protein, 111 aa, synthetic construct
  REGION 1..111: note = VL domain of 9792 IgG histidine scanning variant #12
EIVMTQSPSTLSASVGDRVIITCQSSKSVYNDNWHSWYQQKPGKAPKLLIYGASTLASGVPSRFSGSGSGAEFTLTISSLQPDDFATYYCAGGYSSSSDNGFGQGTKLTVL

SEQ ID NO: 780 - protein, 111 aa, synthetic construct
  REGION 1..111: note = VL domain of 9792 IgG histidine scanning variant #13
EIVMTQSPSTLSASVGDRVIITCQSSKSVYNDNWLHWYQQKPGKAPKLLIYGASTLASGVPSRFSGSGSGAEFTLTISSLQPDDFATYYCAGGYSSSSDNGFGQGTKLTVL

SEQ ID NO: 781 - protein, 111 aa, synthetic construct
  REGION 1..111: note = VL domain of 9792 IgG histidine scanning variant #14
EIVMTQSPSTLSASVGDRVIITCQSSKSVYNDNWLSWYQQKPGKAPKLLIYHASTLASGVPSRFSGSGSGAEFTLTISSLQPDDFATYYCAGGYSSSSDNGFGQGTKLTVL

SEQ ID NO: 782 - protein, 111 aa, synthetic construct
  REGION 1..111: note = VL domain of 9792 IgG histidine scanning variant #15
EIVMTQSPSTLSASVGDRVIITCQSSKSVYNDNWLSWYQQKPGKAPKLLIYGHSTLASGVPSRFSGSGSGAEFTLTISSLQPDDFATYYCAGGYSSSSDNGFGQGTKLTVL

SEQ ID NO: 783 - protein, 111 aa, synthetic construct
  REGION 1..111: note = VL domain of 9792 IgG histidine scanning variant #16
EIVMTQSPSTLSASVGDRVIITCQSSKSVYNDNWLSWYQQKPGKAPKLLIYGAHTLASGVPSRFSGSGSGAEFTLTISSLQPDDFATYYCAGGYSSSSDNGFGQGTKLTVL

SEQ ID NO: 784 - protein, 111 aa, synthetic construct
  REGION 1..111: note = VL domain of 9792 IgG histidine scanning variant #17
EIVMTQSPSTLSASVGDRVIITCQSSKSVYNDNWLSWYQQKPGKAPKLLIYGASHLASGVPSRFSGSGSGAEFTLTISSLQPDDFATYYCAGGYSSSSDNGFGQGTKLTVL

SEQ ID NO: 785 - protein, 111 aa, synthetic construct
  REGION 1..111: note = VL domain of 9792 IgG histidine scanning variant #18
EIVMTQSPSTLSASVGDRVIITCQSSKSVYNDNWLSWYQQKPGKAPKLLIYGASTHASGVPSRFSGSGSGAEFTLTISSLQPDDFATYYCAGGYSSSSDNGFGQGTKLTVL

SEQ ID NO: 786 - protein, 111 aa, synthetic construct
  REGION 1..111: note = VL domain of 9792 IgG histidine scanning variant #19
EIVMTQSPSTLSASVGDRVIITCQSSKSVYNDNWLSWYQQKPGKAPKLLIYGASTLHSGVPSRFSGSGSGAEFTLTISSLQPDDFATYYCAGGYSSSSDNGFGQGTKLTVL

SEQ ID NO: 787 - protein, 111 aa, synthetic construct
  REGION 1..111: note = VL domain of 9792 IgG histidine scanning variant #20
EIVMTQSPSTLSASVGDRVIITCQSSKSVYNDNWLSWYQQKPGKAPKLLIYGASTLAHGVPSRFSGSGSGAEFTLTISSLQPDDFATYYCAGGYSSSSDNGFGQGTKLTVL

SEQ ID NO: 788 - protein, 111 aa, synthetic construct
  REGION 1..111: note = VL domain of 9792 IgG histidine scanning variant #21
EIVMTQSPSTLSASVGDRVIITCQSSKSVYNDNWLSWYQQKPGKAPKLLIYGASTLASGVPSRFSGSGSGAEFTLTISSLQPDDFATYYCHGGYSSSSDNGFGQGTKLTVL

SEQ ID NO: 789 - protein, 111 aa, synthetic construct
  REGION 1..111: note = VL domain of 9792 IgG histidine scanning variant #22
EIVMTQSPSTLSASVGDRVIITCQSSKSVYNDNWLSWYQQKPGKAPKLLIYGASTLASGVPSRFSGSGSGAEFTLTISSLQPDDFATYYCAHGYSSSSDNGFGQGTKLTVL

SEQ ID NO: 790 - protein, 111 aa, synthetic construct
  REGION 1..111: note = VL domain of 9792 IgG histidine scanning variant #23
EIVMTQSPSTLSASVGDRVIITCQSSKSVYNDNWLSWYQQKPGKAPKLLIYGASTLASGVPSRFSGSGSGAEFTLTISSLQPDDFATYYCAGHYSSSSDNGFGQGTKLTVL

SEQ ID NO: 791 - protein, 111 aa, synthetic construct
  REGION 1..111: note = VL domain of 9792 IgG histidine scanning variant #24
EIVMTQSPSTLSASVGDRVIITCQSSKSVYNDNWLSWYQQKPGKAPKLLIYGASTLASGVPSRFSGSGSGAEFTLTISSLQPDDFATYYCAGGHSSSSDNGFGQGTKLTVL

SEQ ID NO: 792 - protein, 111 aa, synthetic construct
  REGION 1..111: note = VL domain of 9792 IgG histidine scanning variant #25
EIVMTQSPSTLSASVGDRVIITCQSSKSVYNDNWLSWYQQKPGKAPKLLIYGASTLASGVPSRFSGSGSGAEFTLTISSLQPDDFATYYCAGGYHSSSDNGFGQGTKLTVL

SEQ ID NO: 793 - protein, 111 aa, synthetic construct
  REGION 1..111: note = VL domain of 9792 IgG histidine scanning variant #26
EIVMTQSPSTLSASVGDRVIITCQSSKSVYNDNWLSWYQQKPGKAPKLLIYGASTLASGVPSRFSGSGSGAEFTLTISSLQPDDFATYYCAGGYSHSSDNGFGQGTKLTVL

SEQ ID NO: 794 - protein, 111 aa, synthetic construct
  REGION 1..111: note = VL domain of 9792 IgG histidine scanning variant #27
EIVMTQSPSTLSASVGDRVIITCQSSKSVYNDNWLSWYQQKPGKAPKLLIYGASTLASGVPSRFSGSGSGAEFTLTISSLQPDDFATYYCAGGYSSHSDNGFGQGTKLTVL

SEQ ID NO: 795 - protein, 111 aa, synthetic construct
  REGION 1..111: note = VL domain of 9792 IgG histidine scanning variant #28
EIVMTQSPSTLSASVGDRVIITCQSSKSVYNDNWLSWYQQKPGKAPKLLIYGASTLASGVPSRFSGSGSGAEFTLTISSLQPDDFATYYCAGGYSSSHDNGFGQGTKLTVL

SEQ ID NO: 796 - protein, 111 aa, synthetic construct
  REGION 1..111: note = VL domain of 9792 IgG histidine scanning variant #29
EIVMTQSPSTLSASVGDRVIITCQSSKSVYNDNWLSWYQQKPGKAPKLLIYGASTLASGVPSRFSGSGSGAEFTLTISSLQPDDFATYYCAGGYSSSSHNGFGQGTKLTVL

SEQ ID NO: 797 - protein, 111 aa, synthetic construct
  REGION 1..111: note = VL domain of 9792 IgG histidine scanning variant #30
EIVMTQSPSTLSASVGDRVIITCQSSKSVYNDNWLSWYQQKPGKAPKLLIYGASTLASGVPSRFSGSGSGAEFTLTISSLQPDDFATYYCAGGYSSSSDHGFGQGTKLTVL

SEQ ID NO: 798 - protein, 111 aa, synthetic construct
  REGION 1..111: note = VL domain of 9792 IgG histidine scanning variant #31
EIVMTQSPSTLSASVGDRVIITCQSSKSVYNDNWLSWYQQKPGKAPKLLIYGASTLASGVPSRFSGSGSGAEFTLTISSLQPDDFATYYCAGGYSSSSDNHFGQGTKLTVL

SEQ ID NO: 799 - protein, 111 aa, synthetic construct
  REGION 1..111: note = VL domain of 9792 IgG histidine scanning variant #32
EIVMTQSPSTLSASVGDRVIITCQSSKSVYNDNWLSWYQQKPGKAPKLLIYGASTLASGVPSRFSGSGSGAEFTLTISSLQPDDFATYYCAGGYSSSSDNGFGQGTKLEIK

SEQ ID NO: 800 - protein, 111 aa, synthetic construct
  REGION 1..111: note = VL domain of 9792 IgG histidine scanning variant #33
EIVMTQSPSTLSASVGDRVIITCQSSKSVYNANWLSWYQQKPGKAPKLLIYGASTLASGVPSRFSGSGSGAEFTLTISSLQPDDFATYYCAGGYSSSSDNGFGQGTKLTVL

SEQ ID NO: 801 - protein, 111 aa, synthetic construct
  REGION 1..111: note = VL domain of 9792 IgG histidine scanning variant #34
EIVMTQSPSTLSASVGDRVIITCQSSKSVYNDNWLSWYQQKPGKAPKLLIYGASTLASGVPSRFSGSGSGAEFTLTISSLQPDDFATYYCAGGYSSSSANGFGQGTKLTVL

SEQ ID NO: 802 - protein, 111 aa, synthetic construct
  REGION 1..111: note = VL domain of 9792 IgG histidine scanning variant #35
EIVMTQSPSTLSASVGDRVIITCQSSKSVYNDNWLSWYQQKPGKAPKLLIYGASTLASGVPSRFSGSGSGAEFTLTISSLQPDDFATYYCAGGYSSSSDAGFGQGTKLTVL

SEQ ID NO: 803 - protein, 119 aa, synthetic construct
  REGION 1..119: note = VH domain of 9797 IgG
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYFCARSFSDISDLIKLWGQGTLVTVSS

SEQ ID NO: 804 - protein, 113 aa, synthetic construct
  REGION 1..113: note = VL domain of 9797 IgG
EIVMTQSPSTLSASVGDRVIITCQASQSLYNNKDLAWYQQKPGKAPKLLIYEASKLASGVPSRFSGSGSGAEFTLTISSLQPDDFATYYCLGEFSCGSGDCLAFGQGTKLTVL

SEQ ID NO: 805 - protein, 10 aa, synthetic construct
  REGION 1..10: note = Heavy Chain CDR1 of 9797 IgG
GIDLSNYAVI

SEQ ID NO: 806 - protein, 16 aa, synthetic construct
  REGION 1..16: note = Heavy Chain CDR2 of 9797 IgG
IIYTYDTTYYANWAKG

SEQ ID NO: 807 - protein, 13 aa, synthetic construct
  REGION 1..13: note = Heavy Chain CDR3 of 9797 IgG
ARSFSDISDLIKL

SEQ ID NO: 808 - protein, 13 aa, synthetic construct
  REGION 1..13: note = Light Chain CDR1 of 9797 IgG
QASQSLYNNKDLA

SEQ ID NO: 809 - protein, 7 aa, synthetic construct
  REGION 1..7: note = Light Chain CDR2 of 9797 IgG
EASKLAS

SEQ ID NO: 810 - protein, 13 aa, synthetic construct
  REGION 1..13: note = Light Chain CDR3 of 9797 IgG
LGEFSCGSGDCLA

SEQ ID NO: 811 - protein, 119 aa, synthetic construct
  REGION 1..119: note = VH domain of 9797 IgG histidine scanning variant #1
EVQLVESGGGLVQPGGSLRLSCTASHIDLSNYAVIWVRQAPGKGLEWVGIIYTYDTTYYANWAKGRFTISRDTSKNTVYLQMNSLRAEDTAVYFCARSFSDISDLIKLWGQGTLVTVSS

SEQ ID NO: 812 - protein, 119 aa, synthetic construct
  REGION 1..119: note = VH domain of 9797 IgG histidine scanning variant #2
EVQLVESGGGLVQPGGSLRLSCTASGHDLSNYAVIWVRQAPGKGLEWVGIIYTYDTTYYANWAKGRFTISRDTSKNTVYLQMNSLRAEDTAVYFCARSFSDISDLIKLWGQGTLVTVSS

SEQ ID NO: 813 - protein, 119 aa, synthetic construct
  REGION 1..119: note = VH domain of 9797 IgG histidine scanning variant #3
EVQLVESGGGLVQPGGSLRLSCTASGIHLSNYAVIWVRQAPGKGLEWVGIIYTYDTTYYANWAKGRFTISRDTSKNTVYLQMNSLRAEDTAVYFCARSFSDISDLIKLWGQGTLVTVSS

SEQ ID NO: 814 - protein, 119 aa, synthetic construct
  REGION 1..119: note = VH domain of 9797 IgG histidine scanning variant #4
EVQLVESGGGLVQPGGSLRLSCTASGIDHSNYAVIWVRQAPGKGLEWVGIIYTYDTTYYANWAKGRFTISRDTSKNTVYLQMNSLRAEDTAVYFCARSFSDISDLIKLWGQGTLVTVSS

SEQ ID NO: 815 - protein, 119 aa, synthetic construct
  REGION 1..119: note = VH domain of 9797 IgG histidine scanning variant #5
EVQLVESGGGLVQPGGSLRLSCTASGIDLHNYAVIWVRQAPGKGLEWVGIIYTYDTTYYANWAKGRFTISRDTSKNTVYLQMNSLRAEDTAVYFCARSFSDISDLIKLWGQGTLVTVSS

SEQ ID NO: 816 - protein, 119 aa, synthetic construct
  REGION 1..119: note = VH domain of 9797 IgG histidine scanning variant #6
EVQLVESGGGLVQPGGSLRLSCTASGIDLSHYAVIWVRQAPGKGLEWVGIIYTYDTTYYANWAKGRFTISRDTSKNTVYLQMNSLRAEDTAVYFCARSFSDISDLIKLWGQGTLVTVSS

SEQ ID NO: 817 - protein, 119 aa, synthetic construct
  REGION 1..119: note = VH domain of 9797 IgG histidine scanning variant #7
EVQLVESGGGLVQPGGSLRLSCTASGIDLSNHAVIWVRQAPGKGLEWVGIIYTYDTTYYANWAKGRFTISRDTSKNTVYLQMNSLRAEDTAVYFCARSFSDISDLIKLWGQGTLVTVSS

SEQ ID NO: 818 - protein, 119 aa, synthetic construct
  REGION 1..119: note = VH domain of 9797 IgG histidine scanning variant #8
EVQLVESGGGLVQPGGSLRLSCTASGIDLSNYHVIWVRQAPGKGLEWVGIIYTYDTTYYANWAKGRFTISRDTSKNTVYLQMNSLRAEDTAVYFCARSFSDISDLIKLWGQGTLVTVSS

SEQ ID NO: 819 - protein, 119 aa, synthetic construct
  REGION 1..119: note = VH domain of 9797 IgG histidine scanning variant #9
EVQLVESGGGLVQPGGSLRLSCTASGIDLSNYAHIWVRQAPGKGLEWVGIIYTYDTTYYANWAKGRFTISRDTSKNTVYLQMNSLRAEDTAVYFCARSFSDISDLIKLWGQGTLVTVSS

SEQ ID NO: 820 - protein, 119 aa, synthetic construct
  REGION 1..119: note = VH domain of 9797 IgG histidine scanning variant #10
EVQLVESGGGLVQPGGSLRLSCTASGIDLSNYAVHWVRQAPGKGLEWVGIIYTYDTTYYANWAKGRFTISRDTSKNTVYLQMNSLRAEDTAVYFCARSFSDISDLIKLWGQGTLVTVSS

SEQ ID NO: 821 - protein, 119 aa, synthetic construct
  REGION 1..119: note = VH domain of 9797 IgG histidine scanning variant #11
EVQLVESGGGLVQPGGSLRLSCTASGIDLSNYAVIWVRQAPGKGLEWVGHIYTYDTTYYANWAKGRFTISRDTSKNTVYLQMNSLRAEDTAVYFCARSFSDISDLIKLWGQGTLVTVSS

SEQ ID NO: 822 - protein, 119 aa, synthetic construct
  REGION 1..119: note = VH domain of 9797 IgG histidine scanning variant #12
EVQLVESGGGLVQPGGSLRLSCTASGIDLSNYAVIWVRQAPGKGLEWVGIHYTYDTTYYANWAKGRFTISRDTSKNTVYLQMNSLRAEDTAVYFCARSFSDISDLIKLWGQGTLVTVSS

SEQ ID NO: 823 - protein, 119 aa, synthetic construct
  REGION 1..119: note = VH domain of 9797 IgG histidine scanning variant #13
EVQLVESGGGLVQPGGSLRLSCTASGIDLSNYAVIWVRQAPGKGLEWVGIIHTYDTTYYANWAKGRFTISRDTSKNTVYLQMNSLRAEDTAVYFCARSFSDISDLIKLWGQGTLVTVSS

SEQ ID NO: 824 - protein, 119 aa, synthetic construct
  REGION 1..119: note = VH domain of 9797 IgG histidine scanning variant #14
EVQLVESGGGLVQPGGSLRLSCTASGIDLSNYAVIWVRQAPGKGLEWVGIIYHYDTTYYANWAKGRFTISRDTSKNTVYLQMNSLRAEDTAVYFCARSFSDISDLIKLWGQGTLVTVSS

SEQ ID NO: 825 - protein, 119 aa, synthetic construct
  REGION 1..119: note = VH domain of 9797 IgG histidine scanning variant #15
EVQLVESGGGLVQPGGSLRLSCTASGIDLSNYAVIWVRQAPGKGLEWVGIIYTHDTTYYANWAKGRFTISRDTSKNTVYLQMNSLRAEDTAVYFCARSFSDISDLIKLWGQGTLVTVSS

SEQ ID NO: 826 - protein, 119 aa, synthetic construct
  REGION 1..119: note = VH domain of 9797 IgG histidine scanning variant #16
EVQLVESGGGLVQPGGSLRLSCTASGIDLSNYAVIWVRQAPGKGLEWVGIIYTYHTTYYANWAKGRFTISRDTSKNTVYLQMNSLRAEDTAVYFCARSFSDISDLIKLWGQGTLVTVSS

SEQ ID NO: 827 - protein, 119 aa, synthetic construct
  REGION 1..119: note = VH domain of 9797 IgG histidine scanning variant #17
EVQLVESGGGLVQPGGSLRLSCTASGIDLSNYAVIWVRQAPGKGLEWVGIIYTYDHTYYANWAKGRFTISRDTSKNTVYLQMNSLRAEDTAVYFCARSFSDISDLIKLWGQGTLVTVSS

SEQ ID NO: 828 - protein, 119 aa, synthetic construct
  REGION 1..119: note = VH domain of 9797 IgG histidine scanning variant #18
EVQLVESGGGLVQPGGSLRLSCTASGIDLSNYAVIWVRQAPGKGLEWVGIIYTYDTHYYANWAKGRFTISRDTSKNTVYLQMNSLRAEDTAVYFCARSFSDISDLIKLWGQGTLVTVSS

SEQ ID NO: 829 - protein, 119 aa, synthetic construct
  REGION 1..119: note = VH domain of 9797 IgG histidine scanning variant #19
EVQLVESGGGLVQPGGSLRLSCTASGIDLSNYAVIWVRQAPGKGLEWVGIIYTYDTTHYANWAKGRFTISRDTSKNTVYLQMNSLRAEDTAVYFCARSFSDISDLIKLWGQGTLVTVSS

SEQ ID NO: 830 - protein, 119 aa, synthetic construct
  REGION 1..119: note = VH domain of 9797 IgG histidine scanning variant #20
EVQLVESGGGLVQPGGSLRLSCTASGIDLSNYAVIWVRQAPGKGLEWVGIIYTYDTTYHANWAKGRFTISRDTSKNTVYLQMNSLRAEDTAVYFCARSFSDISDLIKLWGQGTLVTVSS

SEQ ID NO: 831 - protein, 119 aa, synthetic construct
  REGION 1..119: note = VH domain of 9797 IgG histidine scanning variant #21
EVQLVESGGGLVQPGGSLRLSCTASGIDLSNYAVIWVRQAPGKGLEWVGIIYTYDTTYYHNWAKGRFTISRDTSKNTVYLQMNSLRAEDTAVYFCARSFSDISDLIKLWGQGTLVTVSS

SEQ ID NO: 832 - protein, 119 aa, synthetic construct
  REGION 1..119: note = VH domain of 9797 IgG histidine scanning variant #22
EVQLVESGGGLVQPGGSLRLSCTASGIDLSNYAVIWVRQAPGKGLEWVGIIYTYDTTYYAHWAKGRFTISRDTSKNTVYLQMNSLRAEDTAVYFCARSFSDISDLIKLWGQGTLVTVSS

SEQ ID NO: 833 - protein, 119 aa, synthetic construct
  REGION 1..119: note = VH domain of 9797 IgG histidine scanning variant #23
EVQLVESGGGLVQPGGSLRLSCTASGIDLSNYAVIWVRQAPGKGLEWVGIIYTYDTTYYANHAKGRFTISRDTSKNTVYLQMNSLRAEDTAVYFCARSFSDISDLIKLWGQGTLVTVSS

SEQ ID NO: 834 - protein, 119 aa, synthetic construct
  REGION 1..119: note = VH domain of 9797 IgG histidine scanning variant #24
EVQLVESGGGLVQPGGSLRLSCTASGIDLSNYAVIWVRQAPGKGLEWVGIIYTYDTTYYANWHKGRFTISRDTSKNTVYLQMNSLRAEDTAVYFCARSFSDISDLIKLWGQGTLVTVSS

SEQ ID NO: 835 - protein, 119 aa, synthetic construct
  REGION 1..119: note = VH domain of 9797 IgG histidine scanning variant #25
EVQLVESGGGLVQPGGSLRLSCTASGIDLSNYAVIWVRQAPGKGLEWVGIIYTYDTTYYANWAHGRFTISRDTSKNTVYLQMNSLRAEDTAVYFCARSFSDISDLIKLWGQGTLVTVSS

SEQ ID NO: 836 - protein, 119 aa, synthetic construct
  REGION 1..119: note = VH domain of 9797 IgG histidine scanning variant #26
EVQLVESGGGLVQPGGSLRLSCTASGIDLSNYAVIWVRQAPGKGLEWVGIIYTYDTTYYANWAKHRFTISRDTSKNTVYLQMNSLRAEDTAVYFCARSFSDISDLIKLWGQGTLVTVSS

SEQ ID NO: 837 - protein, 119 aa, synthetic construct
  REGION 1..119: note = VH domain of 9797 IgG histidine scanning variant #27
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYFCHRSFSDISDLIKLWGQGTLVTVSS

SEQ ID NO: 838 - protein, 119 aa, synthetic construct
  REGION 1..119: note = VH domain of 9797 IgG histidine scanning variant #28
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYFCAHSFSDISDLIKLWGQGTLVTVSS

SEQ ID NO: 839 - protein, 119 aa, synthetic construct
  REGION 1..119: note = VH domain of 9797 IgG histidine scanning variant #29
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYFCARHFSDISDLIKLWGQGTLVTVSS

SEQ ID NO: 840 - protein, 119 aa, synthetic construct
  REGION 1..119: note = VH domain of 9797 IgG histidine scanning variant #30
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYFCARSHSDISDLIKLWGQGTLVTVSS

SEQ ID NO: 841 - protein, 119 aa, synthetic construct
  REGION 1..119: note = VH domain of 9797 IgG histidine scanning variant #31
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYFCARSFHDISDLIKLWGQGTLVTVSS

SEQ ID NO: 842 - protein, 119 aa, synthetic construct
  REGION 1..119: note = VH domain of 9797 IgG histidine scanning variant #32
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYFCARSFSHISDLIKLWGQGTLVTVSS

SEQ ID NO: 843 - protein, 119 aa, synthetic construct
  REGION 1..119: note = VH domain of 9797 IgG histidine scanning variant #33
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYFCARSFSDHSDLIKLWGQGTLVTVSS

SEQ ID NO: 844 - protein, 119 aa, synthetic construct
  REGION 1..119: note = VH domain of 9797 IgG histidine scanning variant #34
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYFCARSFSDIHDLIKLWGQGTLVTVSS

SEQ ID NO: 845 - protein, 119 aa, synthetic construct
  REGION 1..119: note = VH domain of 9797 IgG histidine scanning variant #35
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYFCARSFSDISHLIKLWGQGTLVTVSS

SEQ ID NO: 846 - protein, 119 aa, synthetic construct
  REGION 1..119: note = VH domain of 9797 IgG histidine scanning variant #36
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYFCARSFSDISDHIKLWGQGTLVTVSS

SEQ ID NO: 847 - protein, 119 aa, synthetic construct
  REGION 1..119: note = VH domain of 9797 IgG histidine scanning variant #37
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYFCARSFSDISDLHKLWGQGTLVTVSS

SEQ ID NO: 848 - protein, 119 aa, synthetic construct
  REGION 1..119: note = VH domain of 9797 IgG histidine scanning variant #38
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYFCARSFSDISDLIHLWGQGTLVTVSS

SEQ ID NO: 849 - protein, 119 aa, synthetic construct
  REGION 1..119: note = VH domain of 9797 IgG histidine scanning variant #39
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYFCARSFSDISDLIKHWGQGTLVTVSS

SEQ ID NO: 850 - protein, 119 aa, synthetic construct
  REGION 1..119: note = VH domain of 9797 IgG histidine scanning variant #40
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYYCARSFSDISDLIKLWGQGTLVTVSS

SEQ ID NO: 851 - protein, 119 aa, synthetic construct
  REGION 1..119: note = VH domain of 9797 IgG histidine scanning variant #41
EVQLVESGGGLVQPGGSLRLSCTASGIALSNYAVIWVRQAPGKGLEWVGIIYTYDTTYYANWAKGRFTISRDTSKNTVYLQMNSLRAEDTAVYFCARSFSDISDLIKLWGQGTLVTVSS

SEQ ID NO: 852 - protein, 119 aa, synthetic construct
  REGION 1..119: note = VH domain of 9797 IgG histidine scanning variant #42
EVQLVESGGGLVQPGGSLRLSCTASGIDLSNYAVIWVRQAPGKGLEWVGIIYTYATTYYANWAKGRFTISRDTSKNTVYLQMNSLRAEDTAVYFCARSFSDISDLIKLWGQGTLVTVSS

SEQ ID NO: 853 - protein, 119 aa, synthetic construct
  REGION 1..119: note = VH domain of 9797 IgG histidine scanning variant #43
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYFCARSFSAISDLIKLWGQGTLVTVSS

SEQ ID NO: 854 - protein, 119 aa, synthetic construct
  REGION 1..119: note = VH domain of 9797 IgG histidine scanning variant #44
EVQLVESGGGLVQPGGSLRLSCTASGIDLSNYAVIWVRQAPGKGLEWVGIIYTYDTTYYANWAKGRFTISRDTSKNTVYLQMNSLRAEDTAVYFCARSFSDISALIKLWGQGTLVTVSS

SEQ ID NO: 855 - protein, 113 aa, synthetic construct
  REGION 1..113: note = VL domain of 9797 IgG histidine scanning variant #1
EIVMTQSPSTLSASVGDRVIITCHASQSLYNNKDLAWYQQKPGKAPKLLIYEASKLASGVPSRFSGSGSGAEFTLTISSLQPDDFATYYCLGEFSCGSGDCLAFGQGTKLTVL

SEQ ID NO: 856 - protein, 113 aa, synthetic construct
  REGION 1..113: note = VL domain of 9797 IgG histidine scanning variant #2
EIVMTQSPSTLSASVGDRVIITCQHSQSLYNNKDLAWYQQKPGKAPKLLIYEASKLASGVPSRFSGSGSGAEFTLTISSLQPDDFATYYCLGEFSCGSGDCLAFGQGTKLTVL

SEQ ID NO: 857 - protein, 113 aa, synthetic construct
  REGION 1..113: note = VL domain of 9797 IgG histidine scanning variant #3
EIVMTQSPSTLSASVGDRVIITCQAHQSLYNNKDLAWYQQKPGKAPKLLIYEASKLASGVPSRFSGSGSGAEFTLTISSLQPDDFATYYCLGEFSCGSGDCLAFGQGTKLTVL

SEQ ID NO: 858 - protein, 113 aa, synthetic construct
  REGION 1..113: note = VL domain of 9797 IgG histidine scanning variant #4
EIVMTQSPSTLSASVGDRVIITCQASHSLYNNKDLAWYQQKPGKAPKLLIYEASKLASGVPSRFSGSGSGAEFTLTISSLQPDDFATYYCLGEFSCGSGDCLAFGQGTKLTVL

SEQ ID NO: 859 - protein, 113 aa, synthetic construct
  REGION 1..113: note = VL domain of 9797 IgG histidine scanning variant #5
EIVMTQSPSTLSASVGDRVIITCQASQHLYNNKDLAWYQQKPGKAPKLLIYEASKLASGVPSRFSGSGSGAEFTLTISSLQPDDFATYYCLGEFSCGSGDCLAFGQGTKLTVL

SEQ ID NO: 860 - protein, 113 aa, synthetic construct
  REGION 1..113: note = VL domain of 9797 IgG histidine scanning variant #6
EIVMTQSPSTLSASVGDRVIITCQASQSHYNNKDLAWYQQKPGKAPKLLIYEASKLASGVPSRFSGSGSGAEFTLTISSLQPDDFATYYCLGEFSCGSGDCLAFGQGTKLTVL

SEQ ID NO: 861 - protein, 113 aa, synthetic construct
  REGION 1..113: note = VL domain of 9797 IgG histidine scanning variant #7
EIVMTQSPSTLSASVGDRVIITCQASQSLHNNKDLAWYQQKPGKAPKLLIYEASKLASGVPSRFSGSGSGAEFTLTISSLQPDDFATYYCLGEFSCGSGDCLAFGQGTKLTVL

SEQ ID NO: 862 - protein, 113 aa, synthetic construct
  REGION 1..113: note = VL domain of 9797 IgG histidine scanning variant #8
EIVMTQSPSTLSASVGDRVIITCQASQSLYHNKDLAWYQQKPGKAPKLLIYEASKLASGVPSRFSGSGSGAEFTLTISSLQPDDFATYYCLGEFSCGSGDCLAFGQGTKLTVL

SEQ ID NO: 863 - protein, 113 aa, synthetic construct
  REGION 1..113: note = VL domain of 9797 IgG histidine scanning variant #9
EIVMTQSPSTLSASVGDRVIITCQASQSLYNHKDLAWYQQKPGKAPKLLIYEASKLASGVPSRFSGSGSGAEFTLTISSLQPDDFATYYCLGEFSCGSGDCLAFGQGTKLTVL

SEQ ID NO: 864 - protein, 113 aa, synthetic construct
  REGION 1..113: note = VL domain of 9797 IgG histidine scanning variant #10
EIVMTQSPSTLSASVGDRVIITCQASQSLYNNHDLAWYQQKPGKAPKLLIYEASKLASGVPSRFSGSGSGAEFTLTISSLQPDDFATYYCLGEFSCGSGDCLAFGQGTKLTVL

SEQ ID NO: 865 - protein, 113 aa, synthetic construct
  REGION 1..113: note = VL domain of 9797 IgG histidine scanning variant #11
EIVMTQSPSTLSASVGDRVIITCQASQSLYNNKHLAWYQQKPGKAPKLLIYEASKLASGVPSRFSGSGSGAEFTLTISSLQPDDFATYYCLGEFSCGSGDCLAFGQGTKLTVL

SEQ ID NO: 866 - protein, 113 aa, synthetic construct
  REGION 1..113: note = VL domain of 9797 IgG histidine scanning variant #12
EIVMTQSPSTLSASVGDRVIITCQASQSLYNNKDHAWYQQKPGKAPKLLIYEASKLASGVPSRFSGSGSGAEFTLTISSLQPDDFATYYCLGEFSCGSGDCLAFGQGTKLTVL

SEQ ID NO: 867 - protein, 113 aa, synthetic construct
  REGION 1..113: note = VL domain of 9797 IgG histidine scanning variant #13
EIVMTQSPSTLSASVGDRVIITCQASQSLYNNKDLHWYQQKPGKAPKLLIYEASKLASGVPSRFSGSGSGAEFTLTISSLQPDDFATYYCLGEFSCGSGDCLAFGQGTKLTVL

SEQ ID NO: 868 - protein, 113 aa, synthetic construct
  REGION 1..113: note = VL domain of 9797 IgG histidine scanning variant #14
EIVMTQSPSTLSASVGDRVIITCQASQSLYNNKDLAWYQQKPGKAPKLLIYHASKLASGVPSRFSGSGSGAEFTLTISSLQPDDFATYYCLGEFSCGSGDCLAFGQGTKLTVL

SEQ ID NO: 869 - protein, 113 aa, synthetic construct
  REGION 1..113: note = VL domain of 9797 IgG histidine scanning variant #15
EIVMTQSPSTLSASVGDRVIITCQASQSLYNNKDLAWYQQKPGKAPKLLIYEHSKLASGVPSRFSGSGSGAEFTLTISSLQPDDFATYYCLGEFSCGSGDCLAFGQGTKLTVL

SEQ ID NO: 870 - protein, 113 aa, synthetic construct
  REGION 1..113: note = VL domain of 9797 IgG histidine scanning variant #16
EIVMTQSPSTLSASVGDRVIITCQASQSLYNNKDLAWYQQKPGKAPKLLIYEAHKLASGVPSRFSGSGSGAEFTLTISSLQPDDFATYYCLGEFSCGSGDCLAFGQGTKLTVL

SEQ ID NO: 871 - protein, 113 aa, synthetic construct
  REGION 1..113: note = VL domain of 9797 IgG histidine scanning variant #17
EIVMTQSPSTLSASVGDRVIITCQASQSLYNNKDLAWYQQKPGKAPKLLIYEASHLASGVPSRFSGSGSGAEFTLTISSLQPDDFATYYCLGEFSCGSGDCLAFGQGTKLTVL

SEQ ID NO: 872 - protein, 113 aa, synthetic construct
  REGION 1..113: note = VL domain of 9797 IgG histidine scanning variant #18
EIVMTQSPSTLSASVGDRVIITCQASQSLYNNKDLAWYQQKPGKAPKLLIYEASKHASGVPSRFSGSGSGAEFTLTISSLQPDDFATYYCLGEFSCGSGDCLAFGQGTKLTVL

SEQ ID NO: 873 - protein, 113 aa, synthetic construct
  REGION 1..113: note = VL domain of 9797 IgG histidine scanning variant #19
EIVMTQSPSTLSASVGDRVIITCQASQSLYNNKDLAWYQQKPGKAPKLLIYEASKLHSGVPSRFSGSGSGAEFTLTISSLQPDDFATYYCLGEFSCGSGDCLAFGQGTKLTVL

SEQ ID NO: 874 - protein, 113 aa, synthetic construct
  REGION 1..113: note = VL domain of 9797 IgG histidine scanning variant #20
EIVMTQSPSTLSASVGDRVIITCQASQSLYNNKDLAWYQQKPGKAPKLLIYEASKLAHGVPSRFSGSGSGAEFTLTISSLQPDDFATYYCLGEFSCGSGDCLAFGQGTKLTVL

SEQ ID NO: 875 - protein, 113 aa, synthetic construct
  REGION 1..113: note = VL domain of 9797 IgG histidine scanning variant #21
EIVMTQSPSTLSASVGDRVIITCQASQSLYNNKDLAWYQQKPGKAPKLLIYEASKLASGVPSRFSGSGSGAEFTLTISSLQPDDFATYYCHGEFSCGSGDCLAFGQGTKLTVL

SEQ ID NO: 876 - protein, 113 aa, synthetic construct
  REGION 1..113: note = VL domain of 9797 IgG histidine scanning variant #22
EIVMTQSPSTLSASVGDRVIITCQASQSLYNNKDLAWYQQKPGKAPKLLIYEASKLASGVPSRFSGSGSGAEFTLTISSLQPDDFATYYCLHEFSCGSGDCLAFGQGTKLTVL

SEQ ID NO: 877 - protein, 113 aa, synthetic construct
  REGION 1..113: note = VL domain of 9797 IgG histidine scanning variant #23
EIVMTQSPSTLSASVGDRVIITCQASQSLYNNKDLAWYQQKPGKAPKLLIYEASKLASGVPSRFSGSGSGAEFTLTISSLQPDDFATYYCLGHFSCGSGDCLAFGQGTKLTVL

SEQ ID NO: 878 - protein, 113 aa, synthetic construct
  REGION 1..113: note = VL domain of 9797 IgG histidine scanning variant #24
EIVMTQSPSTLSASVGDRVIITCQASQSLYNNKDLAWYQQKPGKAPKLLIYEASKLASGVPSRFSGSGSGAEFTLTISSLQPDDFATYYCLGEHSCGSGDCLAFGQGTKLTVL

SEQ ID NO: 879 - protein, 113 aa, synthetic construct
  REGION 1..113: note = VL domain of 9797 IgG histidine scanning variant #25
EIVMTQSPSTLSASVGDRVIITCQASQSLYNNKDLAWYQQKPGKAPKLLIYEASKLASGVPSRFSGSGSGAEFTLTISSLQPDDFATYYCLGEFHCGSGDCLAFGQGTKLTVL

SEQ ID NO: 880 - protein, 113 aa, synthetic construct
  REGION 1..113: note = VL domain of 9797 IgG histidine scanning variant #26
EIVMTQSPSTLSASVGDRVIITCQASQSLYNNKDLAWYQQKPGKAPKLLIYEASKLASGVPSRFSGSGSGAEFTLTISSLQPDDFATYYCLGEFSCHSGDCLAFGQGTKLTVL

SEQ ID NO: 881 - protein, 113 aa, synthetic construct
  REGION 1..113: note = VL domain of 9797 IgG histidine scanning variant #27
EIVMTQSPSTLSASVGDRVIITCQASQSLYNNKDLAWYQQKPGKAPKLLIYEASKLASGVPSRFSGSGSGAEFTLTISSLQPDDFATYYCLGEFSCGHGDCLAFGQGTKLTVL

SEQ ID NO: 882 - protein, 113 aa, synthetic construct
  REGION 1..113: note = VL domain of 9797 IgG histidine scanning variant #28
EIVMTQSPSTLSASVGDRVIITCQASQSLYNNKDLAWYQQKPGKAPKLLIYEASKLASGVPSRFSGSGSGAEFTLTISSLQPDDFATYYCLGEFSCGSHDCLAFGQGTKLTVL

SEQ ID NO: 883 - protein, 113 aa, synthetic construct
  REGION 1..113: note = VL domain of 9797 IgG histidine scanning variant #29
EIVMTQSPSTLSASVGDRVIITCQASQSLYNNKDLAWYQQKPGKAPKLLIYEASKLASGVPSRFSGSGSGAEFTLTISSLQPDDFATYYCLGEFSCGSGHCLAFGQGTKLTVL

SEQ ID NO: 884 - protein, 113 aa, synthetic construct
  REGION 1..113: note = VL domain of 9797 IgG histidine scanning variant #30
EIVMTQSPSTLSASVGDRVIITCQASQSLYNNKDLAWYQQKPGKAPKLLIYEASKLASGVPSRFSGSGSGAEFTLTISSLQPDDFATYYCLGEFSCGSGDCHAFGQGTKLTVL

SEQ ID NO: 885 - protein, 113 aa, synthetic construct
  REGION 1..113: note = VL domain of 9797 IgG histidine scanning variant #31
EIVMTQSPSTLSASVGDRVIITCQASQSLYNNKDLAWYQQKPGKAPKLLIYEASKLASGVPSRFSGSGSGAEFTLTISSLQPDDFATYYCLGEFSCGSGDCLHFGQGTKLTVL

SEQ ID NO: 886 - protein, 113 aa, synthetic construct
  REGION 1..113: note = VL domain of 9797 IgG histidine scanning variant #32
EIVMTQSPSTLSASVGDRVIITCQASQSLYNNKDLAWYQQKPGKAPKLLIYEASKLASGVPSRFSGSGSGAEFTLTISSLQPDDFATYYCLGEFSCGSGDCLAFGQGTKLEIK

SEQ ID NO: 887 - protein, 113 aa, synthetic construct
  REGION 1..113: note = VL domain of 9797 IgG histidine scanning variant #33
EIVMTQSPSTLSASVGDRVIITCQASQSLYNNKDLAWYQQKPGKAPKLLIYEASKLASGVPSRFSGSGSGAEFTLTISSLQPDDFATYYCLGEFSSGSGDSLAFGQGTKLTVL

SEQ ID NO: 888 - protein, 113 aa, synthetic construct
  REGION 1..113: note = VL domain of 9797 IgG histidine scanning variant #34
EIVMTQSPSTLSASVGDRVIITCQASQSLYNNKDLAWYQQKPGKAPKLLIYEASKLASGVPSRFSGSGSGAEFTLTISSLQPDDFATYYCLGEFSYGSGDYLAFGQGTKLTVL

SEQ ID NO: 889 - protein, 113 aa, synthetic construct
  REGION 1..113: note = VL domain of 9797 IgG histidine scanning variant #35
EIVMTQSPSTLSASVGDRVIITCQASQSLYNNKALAWYQQKPGKAPKLLIYEASKLASGVPSRFSGSGSGAEFTLTISSLQPDDFATYYCLGEFSCGSGDCLAFGQGTKLTVL

SEQ ID NO: 890 - protein, 113 aa, synthetic construct
  REGION 1..113: note = VL domain of 9797 IgG histidine scanning variant #36
EIVMTQSPSTLSASVGDRVIITCQASQSLYNNKDLAWYQQKPGKAPKLLIYAASKLASGVPSRFSGSGSGAEFTLTISSLQPDDFATYYCLGEFSCGSGDCLAFGQGTKLTVL

SEQ ID NO: 891 - protein, 113 aa, synthetic construct
  REGION 1..113: note = VL domain of 9797 IgG histidine scanning variant #37
EIVMTQSPSTLSASVGDRVIITCQASQSLYNNKDLAWYQQKPGKAPKLLIYEASKLASGVPSRFSGSGSGAEFTLTISSLQPDDFATYYCLGAFSCGSGDCLAFGQGTKLTVL

SEQ ID NO: 892 - protein, 113 aa, synthetic construct
  REGION 1..113: note = VL domain of 9797 IgG histidine scanning variant #38
EIVMTQSPSTLSASVGDRVIITCQASQSLYNNKDLAWYQQKPGKAPKLLIYEASKLASGVPSRFSGSGSGAEFTLTISSLQPDDFATYYCLGEFSCGSGACLAFGQGTKLTVL